(19) United States
(12) Patent Application Publication
(10) Pub. No.: US 2026/0158009 A1
(43) Pub. Date: Jun. 11, 2026

(54) ISOXAZOLIDINES AS RIPK1 INHIBITORS AND USE THEREOF

(71) Applicant: GENZYME CORPORATION, Cambridge, MA (US)

(72) Inventors: Elisabeth DEFOSSA, Frankfurt am Main (DE); Niels GRIESANG, Frankfurt am Main (DE); Uwe HEINELT, Frankfurt am Main (DE); Hans MATTER, Frankfurt am Main (DE); Maria MENDEZ-PEREZ, Frankfurt am Main (DE); Nils RACKELMANN, Frankfurt am Main (DE); Lothar SCHWINK, Frankfurt am Main (DE)

(21) Appl. No.: 19/383,242

(22) Filed: Nov. 7, 2025

Related U.S. Application Data
(63) Continuation of application No. PCT/US2024/028154, filed on May 7, 2024.

(30) Foreign Application Priority Data
May 10, 2023 (EP) 23172665.4

(51) Int. Cl. A61K 31/437; A61K 31/42; A61K 31/422; A61K 31/427; A61K 31/4439; A61K 31/444; A61K 31/497; A61K 31/506; C07D 261/02; C07D 413/08; C07D 413/14; C07D 417/14; C07D 471/04

(52) U.S. Cl. CPC A61K 31/437; A61K 31/42; A61K 31/422; A61K 31/427; A61K 31/4439; A61K 31/444; A61K 31/497; A61K 31/506; C07D 261/02; C07D 413/08; C07D 413/14; C07D 417/14; C07D 471/04

(57) ABSTRACT

The disclosure relates to compound of formula (I)

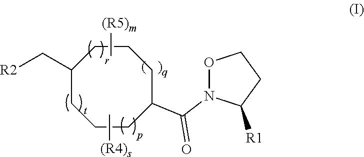

wherein R1 represents a phenyl or a monocyclic heteroaryl, which is optionally substituted by one, two or three R3; R2 represents an aryl or a heteroaryl, which is optionally substituted by one, two or three R6; each R3 is independently chosen from a halogen, a cyano, a (C₁-C₄)alkyl group or a (C₁-C₄)alkoxy group; R4 and R5 are independently chosen from a halogen, a (C₁-C₄)alkyl group, a (C₁-C₄)alkoxy group, or R4 and R5 form together a (C₁-C₄)alkylene bridge; m and s are independently 0 or 1; p, q, r and t are independently 0 or 1; each R6 is independently selected from halogen, cyano, a —NH₂ group, —OH, a (C₁-C₄)alkyl group, —CF₃, —C(O)NH₂, a —C(O)NH—(C₁-C₄)alkyl group, —C(O)OH, a —C(O)O—(C₁-C₄)alkyl group, —SO₂NH₂, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a (C₃-C₆)heterocycloalkyl group, a heteroaryl group and oxo; or a pharmaceutically acceptable salt, solvate, or stereoisomer thereof.

It further relates to the pharmaceutical compositions containing said new compounds.

### RELATED APPLICATIONS

This application is a continuation of International Patent Application No. PCT/US2024/028154, filed May 7, 2024, which claims priority to European Patent Application No. 23172665.4, filed May 10, 2023, the entire disclosures of which are hereby incorporated herein by reference.

### TECHNICAL FIELD

The disclosure relates to new isoxazolidine derivatives useful as a medicament. Said new compounds are in particular useful as kinase inhibitors, and even particularly as RIPK1 inhibitors. They are efficient for treating and/or preventing acute and chronic neurodegenerative diseases like Parkinson's disease, Alzheimer's disease, amyotrophic lateral sclerosis (ALS) or multiple sclerosis (MS).

The disclosure further relates to the pharmaceutical compositions containing said new compounds.

### TECHNICAL BACKGROUND

Although inflammation can be a protective mechanism in response to harmful stimuli such as invasion of pathogens and tissue damages, chronic inflammation is an important underlying factor in many human diseases such as neurodegeneration, rheumatoid arthritis, autoimmune and inflammatory diseases, and cancer. Similarly, the activation of cell death pathways, such as necrosis and apoptosis which are useful in eliminating infected or damaged cells, is also an important underlying mechanism for human diseases, including acute and chronic neurodegenerative diseases. Receptor-interacting protein kinase 1 (UniProtKB Q13546) is a key regulator of inflammation, apoptosis and necroptosis. Receptor-interacting protein kinase 1 has an important role in modulating inflammatory responses mediated by nuclear-factor kappa-light chain enhancer of activated B cells (NF-κB). More recent research has shown that its kinase activity controls necroptosis, a form of necrotic cell death, which was traditionally thought to be passive and unregulated, and is characterized by a unique morphology. Further, receptor-interacting protein kinase 1 is part of a pro-apoptotic complex indicating its activity in regulating apoptosis.

The receptor-interacting protein kinase 1 is subject to complex and intricate regulatory mechanisms, including ubiquitylation, deubiquitylation, and phosphorylation. These regulatory events collectively determine whether a cell will survive and activate an inflammatory response or die through apoptosis or necroptosis. Dysregulation of receptor-interacting protein kinase 1 signalling can lead to excessive inflammation or cell death, and conversely, research has shown that inhibition of receptor-interacting protein kinase 1 can be effective therapies for diseases involving inflammation or cell death.

RIPK1 inhibition has been identified as a promising principle to address different diseases like rheumatoid arthritis (RA), psoriasis, multiple sclerosis, Alzheimer's disease, inflammatory bowel disease such as Crohn's disease, amyotrophic lateral sclerosis (ALS) or ulcerative colitis (UC). To treat some of these diseases like multiple sclerosis (MS) and Alzheimer's disease, access to the central nervous system (CNS) is required, while for other diseases like rheumatoid arthritis, psoriasis, inflammatory bowel disease (IBD) such as Crohn's disease or UC access to the CNS is not essentially required.

Different RIPK1 inhibitors were already described, for example in patent applications WO 2014/125444, WO 2016/185423 or WO 2016/027253 (GSK).

The RIPK1 inhibitor GSK2982772 (oxazepinone derivative disclosed in WO 2014/125444), was evaluated for RA, psoriasis and UC in phase II clinical trials.

Dihydropyrazole compounds with phenyl substituent on dihydropyrazole and a pyrimidine-piperidine element are disclosed as RIPK1 inhibitors by GSK in WO 2018/092089. Other Dihydropyrazole compounds as RIPK1 inhibitors are disclosed in WO2020/224656.

Isoxazolidine compounds with phenyl substituent on isoxazolidine and a pyrimidine-piperidine element are disclosed as RIPK1 inhibitors by GSK in WO 2019/130230. Similar isoxazolidine compounds are disclosed in KR 2020-087922 (Voronoi) and in WO 2020/043173.

Isoxazolidine compounds with a reduced ability to cross the blood-brain-barrier as RIPK1 inhibitors are disclosed in WO 2021/245070.

Compounds with a cycloalkyl element as RIPK1 inhibitor are disclosed in WO 2022/194259.

### SUMMARY

According to one of its objects, the present disclosure relates to a compound of formula (I):

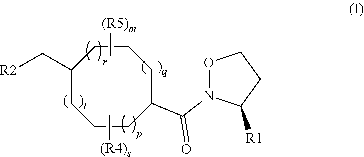

wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge;
m and s are independently 0 or 1;
p, q, r and t are independently 0 or 1;
each R6 is independently selected from halogen, cyano, —OH, a —NH₂ group, a (C₁-C₄)alkyl group, —CF₃, —C(O)NH₂, a —C(O)NH—(C₁-C₄)alkyl group, —C(O)OH, a —C(O)O—(C₁-C₄)alkyl group, —SO₂NH₂, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a (C₃-C₆)heterocycloalkyl group, a heteroaryl group and oxo, wherein said (C₁-C₄)alkyl group, (C₁-C₄)alkoxy group, —O—(C₃-C₆)cycloalkyl group, (C₁-C₄)alkyl group, (C₃-C₆)heterocycloalkyl group or heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

In a related aspect, provided herein pharmaceutical composition comprising a compound of formula (I) or a pharmaceutically acceptable salt, solvate, or stereoisomer thereof, and at least one pharmaceutically acceptable excipient.

In another aspect provided herein is a process for manufacturing a compound of formula (I) and intermediates thereof.

In another aspect, is provided herein a compound of formula (I) or a pharmaceutically acceptable salt, solvate, or stereoisomer thereof, for use as medicament.

In another aspect, is provided herein a compound of formula (I) or a pharmaceutically acceptable salt, solvate, or stereoisomer thereof, for use in the treatment and/or prevention of a disease, disorder or condition that is at least partly mediated by receptor-interacting protein kinase 1.

In another aspect, is provided herein a compound of formula (I), or a pharmaceutically acceptable salt, solvate, or stereoisomer thereof, for use in the treatment and/or prevention of a disease selected from Parkinson's disease, Alzheimer's disease, amyotrophic lateral sclerosis (ALS) or multiple sclerosis (MS).

In another aspect, provided herein is a method of inhibiting receptor-interacting protein kinase 1. Further provided are methods for treating a disease, disorder or condition that is at least partly mediated by receptor-interacting protein kinase 1, comprising administering a therapeutically effective amount of a compound of formula (I) or a pharmaceutical composition as described herein to a subject in need thereof. The disclosure also provides uses of the compounds of formula (I) or compositions thereof in the manufacture of a medicament for the treatment of a disease, disorder or condition that is at least partly mediated by receptor-interacting protein kinase 1.

### DETAILED DESCRIPTION

### Definitions

Unless otherwise stated, the following terms used in the specification and claims have the following meanings set out below.

In this specification the term “alkyl” refers to straight or branched, saturated, aliphatic hydrocarbon groups having the number of atoms indicated. More particularly, a (Cₓ-C_{y}) alkyl group, where x and y are integers, x<y, is a linear or branched saturated aliphatic group comprising from x to y carbon atoms. For example (C₁-C₄)alkyl includes, but is not limited to, methyl, ethyl, n-propyl, isopropyl, n-butyl, isobutyl, sec-butyl and tert-butyl groups, and the like.

The term “alkylene” includes both straight and branched chain divalent alkyl groups. For example, “(C₁-C₄)alkylene” comprises, but is not limited to, methylene (—CH₂—), ethylene (—CH₂CH₂—), methyl methylene (—CH(CH₃)—), propylene, and butylene.

The term “alkoxy” designates an alkyl group singularly bonded to oxygen. In particular, “(Cₓ-C_{y})alkoxy”, as used herein, refers to a —O—(Cₓ-C_{y})alkyl, where x and y are integers, x<y. For example, “(C₁-C₄)alkoxy” comprises, but is not limited to, methoxy, ethoxy, isopropoxy, and t-butoxy.

“Cycloalkyl” refers to saturated or partially unsaturated, optionally substituted, cyclic hydrocarbon groups having the number of atoms indicated. More particularly, a (C₃-C_{z})cycloalkyl group, where z is an integer greater than or equal to 4, comprises from 3 to z carbon atoms. For example, (C₃-C₈)cycloalkyl groups contain from 3 to 8 carbon atoms and are for example cyclopropyl, cyclobutyl, cyclopentyl, cyclohexyl, cycloheptyl or cyclooctyl.

The term “halogen” refers to a chlorine, fluorine, bromine, or iodine atom, and in particular denotes a chlorine or fluorine atom.

The term “heterocyclyl”, “heterocyclic”, or “heterocycle” means a non-aromatic saturated or partially unsaturated, optionally substituted, monocyclic, fused or bridged bicyclic cyclic ring system(s) wherein one to five (suitably one or two) of the carbon atoms are replaced with a heteroatom, such as oxygen, sulfur or nitrogen atom. For example, “(C₃-C₆)heterocycloalkyl group”, as used herein, refers to a (C₃-C₆)cycloalkyl group wherein one or two of the carbon atoms are replaced with a heteroatom, in particular an oxygen or nitrogen atom. Examples of heterocyclic groups include, but are not limited to, cyclic ethers, such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl and the like. Typical sulfur containing heterocycles include tetrahydrothienyl, dihydro-1,3-dithiol, tetrahydro-2H-thiopyran, and hexahydrothiepine. Other heterocycles include, tetrahydro oxazolyl, tetrahydro-oxadiazolyl, tetrahydrodioxazolyl, hexahydrotriazinyl, tetrahydro oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO2 groups are also included. Partially unsaturated heterocyclyl rings contain at least one double bond, such as 1 or 2 double bonds. Examples of partially unsaturated heterocyclyl rings include 1,6-dihydropyridinyl, 1,6-dihydropyridazinyl, and 2,3-dihydropyrrolyl. As the skilled person would appreciate, any heterocycle may be linked to another group via any suitable atom, such as via a carbon or nitrogen atom.

By “bridged ring systems” is meant ring systems in which two rings share more than two atoms, see for example Advanced Organic Chemistry, by Jerry March, 4th Edition, Wiley Interscience, pages 131-133, 1992. In particular, a “bridged (C₆-C₁₀)cycloalkyl” group, as used herein, refers to a bi- or tri-cyclic compound where the cycles are cycloalkyls, the rings share three or more atoms and the bridge contains at least one atom, for example 1 also named C₁-alkylene bridge, 2 also named C₂-alkylene bridge or 3 atoms. Such bridged cycloalkyl groups may be substituted by one or more C₁-C₃ alkyl. Examples are, but not limited to norbornyl or bicyclo[2.2.2]octanyl.

The term “heteroaryl” means an aromatic mono, bi, or polycyclic ring incorporating one or more (for example 1 to 4, particularly 1, 2, or 3) heteroatoms selected from nitrogen, oxygen, or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur, and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. Heteroaryl groups containing nitrogen atoms may be present as the corresponding N-oxides.

Non-limiting examples of heteroaryl groups include furanyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl (pyridinyl), pyridazinyl, pyrimidinyl, pyrazinyl, 1,3,5 triazenyl, benzofuranyl, indolyl, isoindolyl, isoindolinyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzoisoquinolinyl, pyridopyrazinyl, oxazolidinyl, imidazolidinyl, triazolopyridinyl, such as [1,2,4]triazolo[1,5-a]pyridinyl, [1,2,4]triazolo[4,3-a]pyridinyl, pyrrolopyridinyl such as 1H-pyrrolo[2,3-c]pyridinyl, 1H-pyrrolo[3,2-b]pyridinyl or 1H-pyrrolo[3,2-c]pyridinyl, imidazopyridinyl such as imidazo[1,2-a]pyridinyl, imidazopyridazinyl such as imidazo[1,2-b]pyridazinyl, thieno[2,3 b]furanyl, 2H furo[3,2 b]pyranyl, 5H pyrido[2,3 d]o oxazinyl, 1H pyrazolo[4,3 d]oxazolyl, 4H imidazo[4,5 d]thiazolyl, pyrazino[2,3 d]pyridazinyl, imidazo[2,1 b]thiazolyl, imidazo[1,2 b][1,2,4]triazinyl groups, pyrazolopyridinyl such as pyrazolo[4,3-b]pyridinyl and triazolopyridazinyl such as [1,2,4]triazolo[1,5-b]pyridazinyl, benzimidazolyl such as 1H-benzo[d]imidazolyl.

Non-limiting examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl, and tetrazolyl groups.

Non-limiting examples of six membered heteroaryl groups include but are not limited to pyridinyl (pyridyl), pyrazinyl, pyridazinyl, pyrimidinyl, and triazinyl groups.

Non-limiting examples of bicyclic heteroaryl groups containing a six membered ring fused to a five membered ring include but are not limited to triazolopyridinyl such as [1,2,4]triazolo[1,5-a]pyridinyl or [1,2,4]triazolo[4,3-a]pyridinyl, pyrrolopyridinyl such as 1H-pyrrolo[2,3-c]pyridinyl, 1H-pyrrolo[3,2-b]pyridinyl or 1H-pyrrolo[3,2-c]pyridinyl, benzimidazolyl such as 1H-benzo[d]imidazolyl, benzoxazolyl such as oxobenzo[d]oxazol-3(2H)-yl, imidazopyridinyl such as imidazo[1,2-a]pyridinyl, imidazopyridazinyl such as imidazo[1,2-b]pyridazinyl, imidazolyl, indazolyl such as indazol-1-yl or indazol-2-yl, indolyl such as indol-1-yl, isoindolinyl, pyrazolopyridinyl such as pyrazolo[4,3-b]pyridinyl and triazolopyridazinyl such as [1,2,4]triazolo[1,5-b]pyridazinyl. Particular non-limiting examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl, and pteridinyl groups.

The term “aryl” means a cyclic or polycyclic aromatic ring having from 6 to 10 carbon atoms. The term aryl includes both monovalent species and divalent species. Examples of aryl groups include, but are not limited to, phenyl, biphenyl, naphthyl, and the like. Suitably an aryl group is a phenyl.

This specification may also make use of several composite terms to describe groups comprising more than one functionality. Such terms will be understood by a person skilled in the art. For example, heterocyclylC₁-C₄alkyl comprises C₁-C₄alkyl substituted by heterocyclyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups. It is understood that where there are multiple substituents, the substituents chosen may be the same or different.

Where numerical ranges are given, it is understood that the ranges are inclusive of the endpoints.

The phrase “compound of the disclosure” means those compounds which are disclosed herein, both generically and specifically.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The term “pharmaceutically acceptable salts” in this respect refers to the relatively non-toxic, inorganic and organic acid addition salts of compounds of the present disclosure. These salts can be prepared in situ in the administration vehicle or the dosage form manufacturing process or by separately reacting a purified compound of the disclosure in its free base form with a suitable organic or inorganic acid and isolating the salt thus formed during subsequent purification.

As used herein, the term “pharmaceutically acceptable excipient” refers to a substance that aids the administration of an active agent to a subject. By “pharmaceutically acceptable”, it is meant that the excipient is compatible with the other ingredients of the formulation and is not deleterious to the recipient thereof. Pharmaceutical excipients useful in the present disclosure include, but are not limited to, binders, fillers, disintegrants, lubricants, glidants, coatings, sweeteners, flavors and colors.

The phrase “pharmaceutically acceptable carrier” as used herein means a pharmaceutically acceptable material, composition or vehicle, such as a liquid or solid filler, diluent, excipient or solvent encapsulating material, involved in carrying or transporting the subject compound from one organ or portion of the body, to another organ or portion of the body.

“It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder, or condition developing in a human that may be afflicted with or predisposed to the state, disorder, or condition but does not yet experience or display clinical or subclinical symptoms of the state, disorder, or condition, (2) inhibiting the state, disorder, or condition, i.e., arresting, reducing, or delaying the development of the disease or a relapse thereof (in case of maintenance treatment) or at least one clinical or subclinical symptom thereof, or (3) relieving or attenuating the disease, i.e., causing regression of the state, disorder, or condition or at least one of its clinical or subclinical symptoms.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to a human, that has been or will be the object of treatment, observation, or experiment. The methods described herein may be useful in human therapy.

A “therapeutically effective amount” means the amount of a compound that, when administered to a mammal for treating a disease, is sufficient to effect such treatment for the disease. The “therapeutically effective amount” will vary depending on the compound, the disease and its severity, and the age, weight, etc., of the mammal to be treated, which can readily be determined by one of ordinary skill in the art.

As used herein chemical nomenclature as not defined otherwise have the meanings as being used in the technical field.

### Compounds

Disclosed herein is a compound of formula (I):

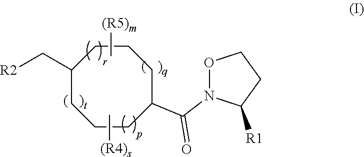

wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge;
m and s are independently 0 or 1;
p, q, r and t are independently 0 or 1;
each R6 is independently selected from halogen, cyano, —OH, a (C₁-C₄)alkyl group, —CF₃, —C(O)NH₂, a —C(O)NH—(C₁-C₄)alkyl group, —C(O)OH, a —C(O)O—(C₁-C₄)alkyl group, —SO₂NH₂, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a heterocycloalkyl group, a heteroaryl group and oxo, wherein said (C₁-C₄)alkyl group, (C₁-C₄)alkoxy group, —O—(C₃-C₆)cycloalkyl group, (C₁-C₄)alkyl group, heterocycloalkyl group or heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

Disclosed herein is a compound of formula (I):

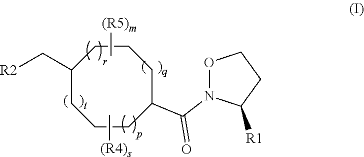

wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge; m and s are independently 0 or 1;
p, q, r and t are independently 0 or 1;
each R6 is independently selected from halogen, cyano, —OH, a —NH₂ group, a (C₁-C₄)alkyl group, —CF₃, —C(O)NH₂, a —C(O)NH—(C₁-C₄)alkyl group, —C(O)OH, a —C(O)O—(C₁-C₄)alkyl group, —SO₂NH₂, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a (C₃-C₆)heterocycloalkyl group, a heteroaryl group and oxo, wherein said (C₁-C₄)alkyl group, (C₁-C₄)alkoxy group, —O—(C₃-C₆)cycloalkyl group, (C₁-C₄)alkyl group, (C₃-C₆)heterocycloalkyl group or heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

According to one embodiment, herein is disclosed a compound of formula (I) wherein:
R1 represents a phenyl or a 5- or 6-membered monocyclic heteroaryl, which is optionally substituted by one, two or three R3;
R2 represents a phenyl, a 5- or 6-membered monocyclic heteroaryl or a 9- or 10-membered bicyclic heteroaryl, which is optionally substituted by one, two or three R6;
each R3 is independently chosen from a halogen, cyano, a (C₁-C₄)alkyl group and a (C₁-C₄)alkoxy group;
R4 and R5 form together a (C₁-C₄)alkylene bridge;
m and s are independently 0 or 1;
p, q, r and t are independently 0 or 1;
each R6 is independently selected from a halogen, cyano, a —NH₂ group, —OH, a (C₁-C₄)alkyl group, —C(O)NH₂, a —C(O)NH—(C₁-C₄)alkyl group, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a 4- or 5-membered heterocycloalkyl, a 5- or 6-membered heteroaryl and oxo, wherein said (C₁-C₄)alkyl group, (C₁-C₄)alkoxy group, —O—(C₃-C₆)cycloalkyl group, heterocycloalkyl group, heteroaryl is optionally substituted by one or two R7;
each R7 is independently —OH, a (C₁-C₄)alkyl group or a (C₁-C₄)alkoxy group,
   or a pharmaceutically acceptable salt, solvate, or stereoisomer thereof.

According to one embodiment, herein is disclosed a compound of formula (I) R1 is selected from furanyl, phenyl, pyrazinyl, pyridinyl, pyrimidinyl and thiazolyl, in particular phenyl or pyridinyl, which is optionally substituted by one, two or three R3;
R2 is selected from benzimidazolyl, benzoxazolyl, imidazolyl, imidazopyridazinyl, imidazopyridinyl, indazolyl, indolyl, isoindolinyl, phenyl, pyrazinyl, pyridazinyl, pyrazolopyridinyl, pyrazolyl, pyridinyl, pyrimidinyl, pyrrolopyridinyl, thiazolyl, triazolopyridazinyl, triazolopyridinyl, triazolyl, in particular phenyl, which is optionally substituted by one, two or three R6;
each R3 is independently chosen from fluorine, cyano, methyl or methoxy;
R4 and R5 form together a methylene bridge or an ethylene bridge;
m and s are independently 0 or 1;
p, q, r and t are independently 0 or 1;
each R6 is independently chosen from chlorine, fluorine, cyano, —OH, methyl, ethyl, —C(O)NH₂, —C(O)NH-methyl, methoxy, ethoxy, oxo, O-methylene-cyclopropyl, cyclopropyloxy, oxetanyloxy, oxetanyl, imidazolyl, pyrazolyl, wherein said ethoxy, methoxy, cyclopropyloxy, ethyl, methyl, oxetanyl, imidazolyl or pyrazolyl is optionally substituted by one or two R7;
each R7 is independently —OH, methyl or methoxy,
   or a pharmaceutically acceptable salt, solvate, or stereoisomer thereof.

According to one embodiment, R1 in formula (I) represents a phenyl or a 5- or 6-membered monocyclic heteroaryl, which is optionally substituted by one, two or three R3.

According to another embodiment, R1 in formula (I) is chosen from phenyl, oxazolyl, pyridinyl, furanyl, pyrazinyl and thiazolyl, in particular from phenyl, pyrazinyl and pyridinyl which is optionally substituted by one, two or three R3.

According to another embodiment, R1 in formula (I) is chosen from phenyl, pyrazinyl and pyridinyl which is optionally substituted by one, two or three R3.

According to another embodiment, R1 in formula (I) is phenyl, which is optionally substituted by one, two or three R3.

According to one embodiment, R1 in formula (I) is unsubstituted. According to another embodiment, R1 in formula (I) is substituted by one, two or three R3, in particular by one or two R3.

According to one embodiment, R2 in formula (I) R2 represents a phenyl, a 5- or 6-membered monocyclic heteroaryl or a 9- or 10-membered bicyclic heteroaryl, which is optionally substituted by one, two or three R6.

According to another embodiment, R2 in formula (I) is selected from benzimidazolyl, such as 1H-benzo[d]imidazolyl, benzoxazolyl, such as benzo[d]oxazol-3(2H)-yl, imidazolyl, imidazopyridazinyl, such as imidazo[1,2-b]pyridazinyl, imidazopyridinyl, such as imidazo[1,2-a]pyridinyl, indazolyl, indolyl, isoindolinyl, phenyl, pyrazinyl, pyridazinyl, pyrazolopyridinyl, such as pyrazolo[4,3-b]pyridinyl, pyrazolyl, pyridinyl, pyrimidinyl, pyrrolopyridinyl, such as 1H-pyrrolo[2,3-c]pyridinyl, 1H-pyrrolo[3,2-b]pyridinyl or 1H-pyrrolo[3,2-c]pyridinyl, thiazolyl, triazolopyridazinyl, such as [1,2,4]triazolo[1,5-b]pyridazinyl, triazolopyridinyl, such as [1,2,4]triazolo[1,5-a]pyridinyl or [1,2,4]triazolo[4,3-a]pyridinyl, triazolyl, such as 1,2,3-triazolyl, pyrazolopyridinyl, imidazopyridinyl, phenyl, pyridazinyl, thiazolyl, which is optionally substituted by one, two or three R6. In particular, R2 is selected from imidazopyridazinyl, such as imidazo[1,2-b]pyridazinyl, indazolyl, pyrazolyl, pyridinyl, pyrimidinyl, triazolopyridazinyl, such as [1,2,4]triazolo[1,5-b]pyridazinyl, triazolopyridinyl, such as [1,2,4]triazolo[1,5-a]pyridinyl or [1,2,4]triazolo[4,3-a]pyridinyl, which is optionally substituted by one, two or three R6.

According to one embodiment, R2 in formula (I) is unsubstituted. According to another embodiment, R2 in formula (I) is substituted by one, two or three R6, in particular by one or two R6.

According to one embodiment, each R3 is independently chosen from a halogen, cyano, a (C₁-C₄)alkyl group and a (C₁-C₄)alkoxy group.

According to another embodiment, each R3 is independently chosen from fluorine, cyano, methyl or methoxy.

According to one embodiment, R4 and R5 in formula (I) form together a (C₁-C₄)alkylene bridge, in particular a methylene or an ethylene bridge.

According to one embodiment, each R6 is independently selected from a halogen, cyano, a —NH₂ group, —OH, a (C₁-C₄)alkyl group, —C(O)NH₂, a —C(O)NH—(C₁-C₄)alkyl group, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a 4- or 5-membered heterocycloalkyl, a 5- or 6-membered heteroaryl and oxo, wherein said (C₁-C₄)alkyl group, (C₁-C₄)alkoxy group, —O—(C₃-C₆)cycloalkyl group, heterocycloalkyl group, heteroaryl is optionally substituted by one, two, three or four R7.

According to another embodiment, each R6 is independently chosen from chlorine, fluorine, cyano, a —NH₂ group, —OH, methyl, ethyl, —C(O)NH₂, —C(O)NH-methyl, methoxy, ethoxy, oxo, O-methylene-cyclopropyl, cyclopropyloxy, oxetanyloxy, oxetanyl, imidazolyl, pyrazolyl, wherein said ethoxy, methoxy, cyclopropyloxy, ethyl, methyl, oxetanyl, imidazolyl or pyrazolyl is optionally substituted by one, two, three or four R7.

According to one embodiment, each R6 is unsubstituted. According to another embodiment, each R6 is substituted by one or two R7.

According to one embodiment, each R7 is independently —OH, a (C₁-C₄)alkyl group or a (C₁-C₄)alkoxy group, in particular each R7 is independently —OH, methyl or methoxy.

According to one embodiment, each R7 is independently —OH, methyl or methoxy.

According to one embodiment, p, q, r and t are independently 0 or 1, wherein at least one of p, q, r and t is 0.

According to one embodiment, p, q, r and t are all equal to 0.

According to this embodiment, a compound of formula (I) is more particularly of following formula (Ia):

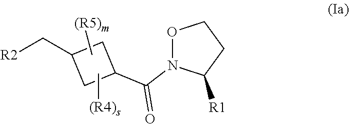

wherein R1, R2, R4, R5, m and s are as defined here-above, or a pharmaceutically acceptable salt, solvate, or stereoisomer thereof.

According to one embodiment, a compound according to the disclosure is of formula (Ia) wherein:
R1 is a phenyl which is optionally substituted by one or two R3;
R2 is a 9-membered bicyclic heteroaryl, in particular a benzimidazolyl, which is optionally substituted by one or two R6;
each R3 is independently chosen from a halogen, cyano or a (C₁-C₄)alkyl group, in particular each R3 is independently chosen from a fluorine, cyano or a methyl;
m and s are 0;
each R6 is a halogen, in particular a fluorine,
or a pharmaceutically acceptable salt, solvate, or stereoisomer thereof.

According to one embodiment, a compound according to the disclosure is of formula (Ia) wherein:
R1 is a phenyl which is optionally substituted by one or two R3;
R2 is a benzimidazolyl, which is optionally substituted by one or two R6;
each R3 is independently chosen from a fluorine, cyano or a methyl;
m and s are 0;
each R6 is a fluorine,
or a pharmaceutically acceptable salt, solvate, or stereoisomer thereof.

According to one embodiment, r, q and t are 0 and p is 1, or r, q and p is 0 and t is 1.

According to this embodiment, a compound of formula (I) is more particularly of following formula (Ib):

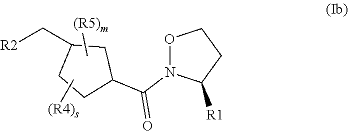

wherein R1, R2, R4, R5, m and s are as defined here-above, or a pharmaceutically acceptable salt, solvate, or stereoisomer thereof.

According to one embodiment, one of q and r is 1 while the other is 0 and one of t and p is 1 while the other is 0.

According to this embodiment, a compound of formula (I) is more particularly of following formula (Ic):

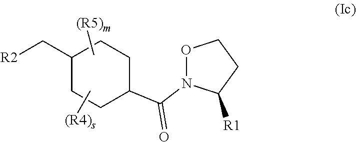

wherein R1, R2, R4, R5, m and s are as defined here-above, or a pharmaceutically acceptable salt, solvate, or stereoisomer thereof.

According to one embodiment, a compound according to the disclosure is of formula (Ic) wherein:
R1 is phenyl or a 6-membered monocyclic heteroaryl, which is optionally substituted by one or two R3, preferably R1 is a phenyl, a pyrazinyl, a pyridinyl or a pyrimidinyl, a thiazolyl, an oxazolyl, which is optionally substituted by one or two R3;
R2 is a 5- or 6-membered monocyclic heteroaryl or a 9-membered bicyclic heteroaryl, optionally substituted by one or two R6; in particular R2 is an imidazopyridazinyl, indazolyl, pyrazolyl, pyridinyl, pyrimidinyl, triazolopyridazinyl, triazolopyridinyl, pyrazolopyridinyl, imidazopyridinyl, phenyl, pyridazinyl, thiazolyl
each R3 is independently chosen from a halogen, cyano or a (C₁-C₄)alkyl group or a (C₁-C₄)alkoxy group, preferably each R3 is independently chosen from fluorine, cyano, methoxy or methyl;
m and s are 0 or m is 0 and s is 1 and R4 is a methyl group;
each R6 is independently chosen from a halogen, a cyano, a —NH₂ group, a (C₁-C₄)alkyl group, a (C₁-C₄)alkoxy group, a —CF₃, a (C₃-C₆)heterocycloalkyl group optionally substituted by one, two, three or four R7, preferably each
R7 is independently a halogen, oxo, —OH, a methyl or a methoxy, in particular each R6 is independently chosen from fluorine, cyano, methyl, methoxy or hydroxyoxetanyl,
or a pharmaceutically acceptable salt, solvate, or stereoisomer thereof.

According to one embodiment, a compound according to the disclosure is of formula (Ic) wherein:
R1 is a phenyl, a pyrazinyl, a pyridinyl, a thiazolyl, an oxazolyl or a pyrimidinyl which is optionally substituted by one or two R3;
R2 is imidazopyridazinyl, indazolyl, pyrazolyl, pyridinyl, pyrimidinyl, triazolopyridazinyl or triazolopyridinyl, pyrazolopyridinyl, imidazopyridinyl, phenyl, pyridazinyl, thiazolyl, optionally substituted by one or two R6;
each R3 is independently chosen from fluorine, cyano, methoxy or methyl;
m and s are 0 or m is 0 and s is 1 and R4 is a methyl group;
each R6 is independently chosen from fluorine, a cyano, a —NH₂ group, methyl, a —CF₃, a methoxy or a hydroxyoxetanyl;
or a pharmaceutically acceptable salt, solvate, or stereoisomer thereof.

According to one embodiment, a compound according to the disclosure is of formula (Ic) wherein:
R1 is a phenyl, which is optionally substituted by one or two R3;
R2 is imidazopyridazinyl, indazolyl, pyrazolyl, pyridinyl, pyrimidinyl, triazolopyridazinyl, triazolopyridinyl, pyrazolopyridinyl, imidazopyridinyl, phenyl, pyridazinyl or thiazolyl, optionally substituted by one or two R6;
each R3 is independently chosen from fluorine, cyano or methyl;
m and s are 0 or m is 0 and s is 1 and R4 is a methyl group;
each R6 is independently chosen from fluorine, cyano, methyl or methoxy;
or a pharmaceutically acceptable salt, solvate, or stereoisomer thereof.

According to one embodiment, t and p are equal to 1 and one of q and r is 1 while the other is 0.

According to this embodiment, a compound of formula (I) is more particularly of following formula (Id):
wherein R1, R2, R4, R5, m and s are as defined here-above, or a pharmaceutically acceptable salt, solvate, or stereoisomer thereof.

According to one embodiment, herein is further disclosed a compound of formula (Ig):

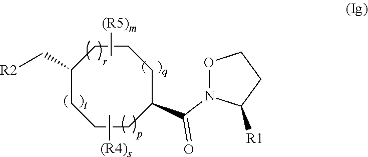

wherein
R1, R2, R4, R5, r, q, p, t, m and s are as defined as in compounds of formula (I), or a pharmaceutically acceptable salt, solvate, or stereoisomer thereof.

According to this embodiment, a compound of formula (I) is more particularly of following formula (Ie):

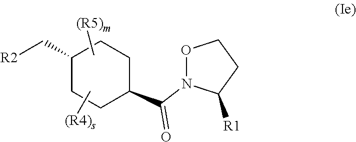

wherein R1, R2, R4, R5, m and s are as defined here-above, or a pharmaceutically acceptable salt, solvate, or stereoisomer thereof.

According to one embodiment, a compound according to the disclosure is of formula (Ie) wherein:
R1 is phenyl or a 6-membered monocyclic heteroaryl, which is optionally substituted by one or two R3, preferably R1 is a phenyl, a pyrazinyl, a pyridinyl or a pyrimidinyl, a thiazolyl, an oxazolyl, which is optionally substituted by one or two R3;
R2 is a 5- or 6-membered monocyclic heteroaryl or a 9-membered bicyclic heteroaryl, optionally substituted by one or two R6; in particular R2 is an imidazopyridazinyl, indazolyl, pyrazolyl, pyridinyl, pyrimidinyl, triazolopyridazinyl, triazolopyridinyl, pyrazolopyridinyl, imidazopyridinyl, phenyl, pyridazinyl, thiazolyl
each R3 is independently chosen from a halogen, cyano or a (C₁-C₄)alkyl group or a (C₁-C₄)alkoxy group, preferably each R3 is independently chosen from fluorine, cyano, methoxy or methyl;
m and s are 0 or m is 0 and s is 1 and R4 is a methyl group;
each R6 is independently chosen from a halogen, a cyano, a —NH₂ group, a (C₁-C₄)alkyl group, a (C₁-C₄)alkoxy group, a —CF₃, a (C₃-C₆)heterocycloalkyl group optionally substituted by one, two, three or four R7, preferably each
R7 is independently a halogen, oxo, —OH, a methyl or a methoxy, R6 is independently chosen from fluorine, cyano, methyl, methoxy or hydroxyoxetanyl,
or a pharmaceutically acceptable salt, solvate, or stereoisomer thereof.

According to one embodiment, a compound according to the disclosure is of formula (Ie) wherein:
R1 is a phenyl, a pyrazinyl, a pyridinyl, a thiazolyl, an oxazolyl or a pyrimidinyl which is optionally substituted by one or two R3;
R2 is imidazopyridazinyl, indazolyl, pyrazolyl, pyridinyl, pyrimidinyl, triazolopyridazinyl or triazolopyridinyl, pyrazolopyridinyl, imidazopyridinyl, phenyl, pyridazinyl, thiazolyl, optionally substituted by one or two R6;
each R3 is independently chosen from fluorine, cyano, methoxy or methyl;
m and s are 0 or m is 0 and s is 1 and R4 is a methyl group;
each R6 is independently chosen from fluorine, a cyano, a —NH₂ group, methyl, a —CF₃, a methoxy or a hydroxyoxetanyl;
or a pharmaceutically acceptable salt, solvate, or stereoisomer thereof.

According to one embodiment, a compound according to the disclosure is of formula (Ie) wherein:
R1 is a phenyl, which is optionally substituted by one or two R3;
R2 is imidazopyridazinyl, indazolyl, pyrazolyl, pyridinyl, pyrimidinyl, triazolopyridazinyl, triazolopyridinyl, pyrazolopyridinyl, imidazopyridinyl, phenyl, pyridazinyl or thiazolyl, optionally substituted by one or two R6;
each R3 is independently chosen from fluorine, cyano or methyl;
m and s are 0 or m is 0 and s is 1 and R4 is a methyl group;
each R6 is independently chosen from fluorine, cyano, methyl or methoxy;
or a pharmaceutically acceptable salt, solvate, or stereoisomer thereof.

According to one embodiment, t and p are equal to 1 and one of q and r is 1 while the other is 0.

According to this embodiment, a compound of formula (I) is more particularly of following formula (Id):
wherein R1, R2, R4, R5, m and s are as defined here-above, or a pharmaceutically acceptable salt, solvate, or stereoisomer thereof.

According to one embodiment, m and s in formula (I) above are equal, namely m and s are 0 or m and s are 1. According to another embodiment at most one of m and s is 1.

According to one embodiment, herein is further disclosed a compound of formula (I) having following formula (Ib), (Ic), in particular of formula (Ie), or (Id):

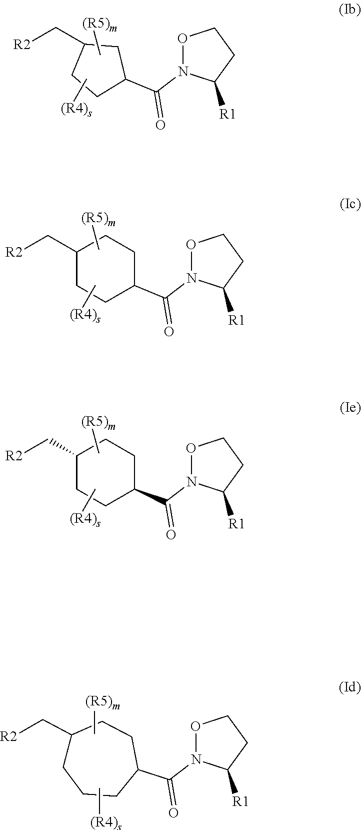

wherein R1, R2, R4, R5, m and s are as defined here-above,
or a pharmaceutically acceptable salt, solvate, or stereoisomer thereof;
in particular having:
formula (Ib),
formula (Ic) and more particularly formula (Ie), or
formula (Id),
or a pharmaceutically acceptable salt, solvate, or stereoisomer thereof;
more particularly having:
formula (Ib),
formula (Ic) and more particularly formula (Ie),
or a pharmaceutically acceptable salt, solvate, or stereoisomer thereof.

According to one embodiment, herein is further disclosed a compound of formula (I) wherein when the group

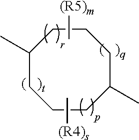

is an optionally substituted cyclobutyl, then R4 and R5 are independently chosen from a halogen, a (C₁-C₄)alkyl group or a (C₁-C₄)alkoxy group.

According to one embodiment, herein is further disclosed a compound of formula (If):

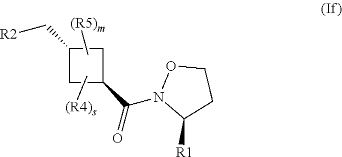

wherein
R1, R2, R4, R5, m and s are as defined here-above, or a pharmaceutically acceptable salt, solvate, or stereoisomer thereof.

The nomenclature of the following compounds (1) to (411) was generated according to the principles of the International Union of Pure and Applied Chemistry. “cis” and “trans” prefixes were also used to assign the relative stereochemistry of two adjacent chiral centers.

According to one embodiment the compound of formula (I) is chosen from:
(1) trans-3-[(3S)-2-[4-[(2-methyl-[1,2,4]triazolo[1,5-a]pyridin-7-yl)methyl]cyclohexanecarbonyl]isoxazolidin-3-yl]benzonitrile;
(2) trans-1-((4-((S)-3-(3-cyano-5-fluorophenyl)isoxazolidine-2-carbonyl)cyclohexyl)methyl)-1H-pyrrolo[3,2-b]pyridine-6-carbonitrile,
(3) trans-1-((4-((S)-3-(5-fluoropyridin-3-yl)isoxazolidine-2-carbonyl)cyclohexyl)methyl)-1H-pyrrolo[3,2-b]pyridine-6-carbonitrile,
(4) trans-1-((4-((S)-3-(5-fluoropyridin-3-yl)isoxazolidine-2-carbonyl)cyclohexyl)methyl)-4-methyl-1H-imidazole-5-carbonitrile,
(5) trans-1-((4-((S)-3-(3-cyano-5-fluorophenyl)isoxazolidine-2-carbonyl)cyclohexyl)methyl)-1H-benzo[d]imidazole-5-carbonitrile,
(6) trans-1-((4-((S)-3-(5-fluoropyridin-3-yl)isoxazolidine-2-carbonyl)cyclohexyl)methyl)-1H-benzo[d]imidazole-5-carbonitrile,
(7) trans-3-((S)-2-(4-((1H-benzo[d]imidazol-1-yl)methyl)cyclohexane-1-carbonyl)isoxazolidin-3-yl)-5-fluorobenzonitrile,
(8) trans-(4-((1H-benzo[d]imidazol-1-yl)methyl)cyclohexyl)((S)-3-(3,5-difluorophenyl)isoxazolidin-2-yl)methanone,
(9) trans-(4-((1H-benzo[d]imidazol-1-yl)methyl)cyclohexyl)((S)-3-(5-fluoropyridin-3-yl)isoxazolidin-2-yl)methanone,
(10) trans-1-((4-((S)-3-(3,5-difluorophenyl)isoxazolidine-2-carbonyl)cyclohexyl)methyl)-6-fluoro-1H-benzo[d]imidazole-5-carbonitrile,
(11) trans-1-((4-((S)-3-(3-cyano-5-fluorophenyl)isoxazolidine-2-carbonyl)cyclohexyl)methyl)-6-fluoro-1H-benzo[d]imidazole-5-carbonitrile,
(12) trans-1-((4-((S)-3-(3,5-difluorophenyl)isoxazolidine-2-carbonyl)cyclohexyl)methyl)-5-fluoro-1H-benzo[d]imidazole-6-carbonitrile,
(13) trans-1-((4-((S)-3-(3-cyano-5-fluorophenyl)isoxazolidine-2-carbonyl)cyclohexyl)methyl)-5-fluoro-1H-benzo[d]imidazole-6-carbonitrile,
(14) trans-3-((S)-2-(4-((1H-pyrrolo[3,2-b]pyridin-1-yl)methyl)cyclohexane-1-carbonyl)isoxazolidin-3-yl)-5-fluorobenzonitrile,
(15) trans-3-((S)-2-(4-((1H-pyrrolo[3,2-c]pyridin-1-yl)methyl)cyclohexane-1-carbonyl)isoxazolidin-3-yl)-5-fluorobenzonitrile,
(16) trans-3-((S)-2-(4-((1H-pyrrolo[2,3-c]pyridin-1-yl)methyl)cyclohexane-1-carbonyl)isoxazolidin-3-yl)-5-fluorobenzonitrile,
(17) trans-1-((4-((S)-3-(3,5-difluorophenyl)isoxazolidine-2-carbonyl)cyclohexyl)methyl)-1H-pyrrolo[3,2-b]pyridine-6-carbonitrile,
(18) trans-3-fluoro-5-((S)-2-(4-((2-oxobenzo[d]oxazol-3(2H)-yl)methyl)cyclohexane-1-carbonyl)isoxazolidin-3-yl)benzonitrile,
(19) trans-3-((S)-2-(4-((6-chloro-1H-pyrrolo[3,2-b]pyridin-1-yl)methyl)cyclohexane-1-carbonyl)isoxazolidin-3-yl)-5-fluorobenzonitrile,
(20) trans-3-((4-((S)-3-(3,5-difluorophenyl)isoxazolidine-2-carbonyl)cyclohexyl)methyl)benzo[d]oxazol-2(3H)-one,
(21) trans-(4-((6-chloro-1H-pyrrolo[3,2-b]pyridin-1-yl)methyl)cyclohexyl)((S)-3-(3,5-difluorophenyl)isoxazolidin-2-yl)methanone,
(22) trans-1-((4-((S)-3-(3-cyano-5-fluorophenyl)isoxazolidine-2-carbonyl)cyclohexyl)methyl)-5-fluoro-1H-indole-6-carbonitrile,
(23) trans-1-((4-((S)-3-(3,5-difluorophenyl)isoxazolidine-2-carbonyl)cyclohexyl)methyl)-5-fluoro-1H-indole-6-carbonitrile,
(24) trans-2-((4-((S)-3-(3-cyano-5-fluorophenyl)isoxazolidine-2-carbonyl)cyclohexyl)methyl)-2H-pyrazolo[4,3-b]pyridine-6-carbonitrile,
(25) trans-2-((4-((S)-3-(3,5-difluorophenyl)isoxazolidine-2-carbonyl)cyclohexyl)methyl)-2H-pyrazolo[4,3-b]pyridine-6-carbonitrile,
(26) trans-1-((4-((S)-3-(3,5-difluorophenyl)isoxazolidine-2-carbonyl)cyclohexyl)methyl)-1H-pyrazolo[4,3-b]pyridine-6-carbonitrile,
(27) trans-1-((4-((S)-3-(3-cyano-5-fluorophenyl)isoxazolidine-2-carbonyl)cyclohexyl)methyl)-1H-pyrazolo[4,3-b]pyridine-6-carbonitrile,
(28) trans-1-((4-((S)-3-(3-cyano-5-fluorophenyl)isoxazolidine-2-carbonyl)cyclohexyl)methyl)-1H-indazole-6-carbonitrile,
(29) trans-1-((4-((S)-3-(3,5-difluorophenyl)isoxazolidine-2-carbonyl)cyclohexyl)methyl)-1H-indazole-6-carbonitrile,
(30) trans-2-((4-((S)-3-(3-cyano-5-fluorophenyl)isoxazolidine-2-carbonyl)cyclohexyl)methyl)-2H-indazole-6-carbonitrile,
(31) trans-2-((4-((S)-3-(3,5-difluorophenyl)isoxazolidine-2-carbonyl)cyclohexyl)methyl)-2H-indazole-6-carbonitrile,
(32) trans-1-((4-((S)-3-(3-cyano-5-fluorophenyl)isoxazolidine-2-carbonyl)cyclohexyl)methyl)-4-fluoro-1H-indazole-6-carbonitrile,
(33) trans-1-((4-((S)-3-(3,5-difluorophenyl)isoxazolidine-2-carbonyl)cyclohexyl)methyl)-4-fluoro-1H-indazole-6-carbonitrile,
(34) trans-2-((4-((S)-3-(3-cyano-5-fluorophenyl)isoxazolidine-2-carbonyl)cyclohexyl)methyl)-4-fluoro-2H-indazole-6-carbonitrile,
(35) trans-2-((4-((S)-3-(3,5-difluorophenyl)isoxazolidine-2-carbonyl)cyclohexyl)methyl)-4-fluoro-2H-indazole-6-carbonitrile,
(36) trans-1-((4-((S)-3-(3-cyano-5-fluorophenyl)isoxazolidine-2-carbonyl)cyclohexyl)methyl)-4-fluoro-1H-benzo[d]imidazole-6-carbonitrile,
(37) trans-1-((4-((S)-3-(3,5-difluorophenyl)isoxazolidine-2-carbonyl)cyclohexyl)methyl)-4-fluoro-1H-benzo[d]imidazole-6-carbonitrile,
(38) trans-4-fluoro-1-((4-((S)-3-(5-methylfuran-3-yl)isoxazolidine-2-carbonyl)cyclohexyl)methyl)-1H-benzo[d]imidazole-6-carbonitrile,
(39) trans-5-fluoro-1-((4-((S)-3-(5-methylfuran-3-yl)isoxazolidine-2-carbonyl)cyclohexyl)methyl)-1H-benzo[d]imidazole-6-carbonitrile,
(40) trans-6-fluoro-1-((4-((S)-3-(5-methylfuran-3-yl)isoxazolidine-2-carbonyl)cyclohexyl)methyl)-1H-benzo[d]imidazole-5-carbonitrile,
(41) trans-2-((4-((S)-3-(5-methylfuran-3-yl)isoxazolidine-2-carbonyl)cyclohexyl)methyl)-2H-indazole-6-carbonitrile,
(42) trans-4-fluoro-2-((4-((S)-3-(5-methylfuran-3-yl)isoxazolidine-2-carbonyl)cyclohexyl)methyl)-2H-indazole-6-carbonitrile,
(43) trans-1-((4-((S)-3-(5-methylfuran-3-yl)isoxazolidine-2-carbonyl)cyclohexyl)methyl)-1H-indazole-6-carbonitrile,
(44) trans-4-fluoro-1-((4-((S)-3-(5-methylfuran-3-yl)isoxazolidine-2-carbonyl)cyclohexyl)methyl)-1H-indazole-6-carbonitrile,
(45) trans-1-((4-((S)-3-(3,5-difluorophenyl)isoxazolidine-2-carbonyl)cyclohexyl)methyl)-4-fluoro-1H-indole-6-carbonitrile,
(46) trans-1-((4-((S)-3-(3-cyano-5-fluorophenyl)isoxazolidine-2-carbonyl)cyclohexyl)methyl)-4-fluoro-1H-indole-6-carbonitrile,
(47) trans-4-fluoro-1-((4-((S)-3-(5-methylfuran-3-yl)isoxazolidine-2-carbonyl)cyclohexyl)methyl)-1H-indole-6-carbonitrile,
(48) trans-3-fluoro-5-((S)-2-(4-((5-fluoro-1H-benzo[d]imidazol-1-yl)methyl)cyclohexane-1-carbonyl)isoxazolidin-3-yl)benzonitrile,
(49) trans-((S)-3-(3,5-difluorophenyl)isoxazolidin-2-yl)(4-((5-fluoro-1H-benzo[d]imidazol-1-yl)methyl)cyclohexyl)methanone,
(50) trans-3-fluoro-5-((S)-2-(4-((5-fluoro-2-methyl-1H-benzo[d]imidazol-1-yl)methyl)cyclohexane-1-carbonyl)isoxazolidin-3-yl)benzonitrile,
(51) trans-((S)-3-(3,5-difluorophenyl)isoxazolidin-2-yl)(4-((5-fluoro-2-methyl-1H-benzo[d]imidazol-1-yl)methyl)cyclohexyl)methanone,
(52) trans-1-((4-((S)-3-(3,5-difluorophenyl)isoxazolidine-2-carbonyl)cyclohexyl)methyl)-1H-indazole-5-carbonitrile,
(53) trans-1-((4-((S)-3-(3-cyano-5-fluorophenyl)isoxazolidine-2-carbonyl)cyclohexyl)methyl)-1H-indazole-5-carbonitrile,
(54) trans-3-fluoro-5-((S)-2-(4-((5-fluoro-1H-indazol-1-yl)methyl)cyclohexane-1-carbonyl)isoxazolidin-3-yl)benzonitrile,
(55) trans-((S)-3-(3,5-difluorophenyl)isoxazolidin-2-yl)(4-((5-fluoro-1H-indazol-1-yl)methyl)cyclohexyl)methanone,
(56) trans-3-fluoro-5-((S)-2-(4-((5-fluoro-2H-indazol-2-yl)methyl)cyclohexane-1-carbonyl)isoxazolidin-3-yl)benzonitrile,
(57) trans-((S)-3-(3,5-difluorophenyl)isoxazolidin-2-yl)(4-((5-fluoro-2H-indazol-2-yl)methyl)cyclohexyl)methanone,
(58) trans-2-((4-((S)-3-(3,5-difluorophenyl)isoxazolidine-2-carbonyl)cyclohexyl)methyl)-2H-indazole-5-carbonitrile,
(59) trans-2-((4-((S)-3-(3-cyano-5-fluorophenyl)isoxazolidine-2-carbonyl)cyclohexyl)methyl)-2H-indazole-5-carbonitrile,
(60) trans-((S)-3-(3,5-difluorophenyl)isoxazolidin-2-yl)(4-((3-methyl-1H-indazol-1-yl)methyl)cyclohexyl)methanone,
(61) trans-3-fluoro-5-((S)-2-(4-((3-methyl-1H-indazol-1-yl)methyl)cyclohexane-1-carbonyl)isoxazolidin-3-yl)benzonitrile,
(62) trans-((S)-3-(3,5-difluorophenyl)isoxazolidin-2-yl)(4-((3-methyl-2H-indazol-2-yl)methyl)cyclohexyl)methanone,
(63) trans-3-fluoro-5-((S)-2-(4-((3-methyl-2H-indazol-2-yl)methyl)cyclohexane-1-carbonyl)isoxazolidin-3-yl)benzonitrile,
(64) trans-1-((4-((S)-3-(5-cyanopyridin-3-yl)isoxazolidine-2-carbonyl)cyclohexyl)methyl)-5-fluoro-1H-benzo[d]imidazole-6-carbonitrile,
(65) trans-1-((4-((S)-3-(5-cyanopyridin-3-yl)isoxazolidine-2-carbonyl)cyclohexyl)methyl)-6-fluoro-1H-benzo[d]imidazole-5-carbonitrile,
(66) trans-1-((4-((S)-3-(3-cyano-5-fluorophenyl)isoxazolidine-2-carbonyl)cyclohexyl)methyl)-6-fluoro-1H-indazole-5-carbonitrile,
(67) trans-1-((4-((S)-3-(3,5-difluorophenyl)isoxazolidine-2-carbonyl)cyclohexyl)methyl)-6-fluoro-1H-indazole-5-carbonitrile,
(68) trans-1-((4-((S)-3-(3,5-difluorophenyl)isoxazolidine-2-carbonyl)cyclohexyl)methyl)-6-fluoro-3-methyl-1H-indazole-5-carbonitrile,
(69) trans-1-((4-((S)-3-(3-cyano-5-fluorophenyl)isoxazolidine-2-carbonyl)cyclohexyl)methyl)-6-fluoro-3-methyl-1H-indazole-5-carbonitrile,
(70) trans-1-((4-((S)-3-(3-cyano-5-fluorophenyl)isoxazolidine-2-carbonyl)cyclohexyl)methyl)-5-fluoro-1H-benzo[d]imidazole-6-carboxamide,
(71) trans-1-((4-((S)-3-(3,5-difluorophenyl)isoxazolidine-2-carbonyl)cyclohexyl)methyl)-5-fluoro-1H-benzo[d]imidazole-6-carboxamide,
(72) trans-1-((4-((S)-3-(3-cyano-5-fluorophenyl)isoxazolidine-2-carbonyl)cyclohexyl)methyl)-1H-indazole-5-carboxamide,
(73) trans-1-((4-((S)-3-(3,5-difluorophenyl)isoxazolidine-2-carbonyl)cyclohexyl)methyl)-1H-indazole-5-carboxamide,
(74) trans-1-((4-((S)-3-(3-cyano-5-fluorophenyl)isoxazolidine-2-carbonyl)cyclohexyl)methyl)-6-fluoro-1H-benzo[d]imidazole-5-carboxamide,
(75) trans-1-((4-((S)-3-(3,5-difluorophenyl)isoxazolidine-2-carbonyl)cyclohexyl)methyl)-6-fluoro-1H-benzo[d]imidazole-5-carboxamide,
(76) trans-1-((4-((S)-3-(3-cyano-5-fluorophenyl)isoxazolidine-2-carbonyl)cyclohexyl)methyl)-5-fluoro-1H-indole-6-carboxamide,
(77) trans-1-((4-((S)-3-(3,5-difluorophenyl)isoxazolidine-2-carbonyl)cyclohexyl)methyl)-5-fluoro-1H-indole-6-carboxamide,
(78) trans-1-((4-((S)-3-(3-cyano-5-fluorophenyl)isoxazolidine-2-carbonyl)cyclohexyl)methyl)-1H-pyrazolo[4,3-b]pyridine-6-carboxamide,
(79) trans-1-((4-((S)-3-(3,5-difluorophenyl)isoxazolidine-2-carbonyl)cyclohexyl)methyl)-1H-pyrazolo[4,3-b]pyridine-6-carboxamide,
(80) trans-1-((4-((S)-3-(3-cyano-5-fluorophenyl)isoxazolidine-2-carbonyl)cyclohexyl)methyl)-4-fluoro-1H-benzo[d]imidazole-6-carboxamide,
(81) trans-1-((4-((S)-3-(3,5-difluorophenyl)isoxazolidine-2-carbonyl)cyclohexyl)methyl)-4-fluoro-1H-benzo[d]imidazole-6-carboxamide,
(82) trans-3-((S)-2-(4-((4,5-dimethyl-1H-imidazol-1-yl)methyl)cyclohexane-1-carbonyl)isoxazolidin-3-yl)-5-fluorobenzonitrile,
(83) trans-((S)-3-(3,5-difluorophenyl)isoxazolidin-2-yl)(4-((4,5-dimethyl-1H-imidazol-1-yl)methyl)cyclohexyl)methanone,
(84) trans-1-((4-((S)-3-(3,5-difluorophenyl)isoxazolidine-2-carbonyl)cyclohexyl)methyl)-5-fluoro-3-methyl-1H-indazole-6-carbonitrile,
(85) trans-1-((4-((S)-3-(3-cyano-5-fluorophenyl)isoxazolidine-2-carbonyl)cyclohexyl)methyl)-5-fluoro-3-methyl-1H-indazole-6-carbonitrile,
(86) trans-1-((4-((S)-3-(3,5-difluorophenyl)isoxazolidine-2-carbonyl)cyclohexyl)methyl)-5-fluoro-1H-indazole-6-carbonitrile,
(87) trans-1-((4-((S)-3-(3-cyano-5-fluorophenyl)isoxazolidine-2-carbonyl)cyclohexyl)methyl)-5-fluoro-1H-indazole-6-carbonitrile,
(88) trans-1-((4-((S)-3-(3-cyano-5-fluorophenyl)isoxazolidine-2-carbonyl)cyclohexyl)methyl)-4-methyl-1H-imidazole-5-carbonitrile,
(89) trans-1-((4-((S)-3-(3,5-difluorophenyl)isoxazolidine-2-carbonyl)cyclohexyl)methyl)-4-methyl-1H-imidazole-5-carbonitrile,
(90) trans-1-((4-((S)-3-(3-cyano-5-fluorophenyl)isoxazolidine-2-carbonyl)cyclohexyl)methyl)-5-methyl-1H-imidazole-4-carbonitrile,
(91) trans-1-((4-((S)-3-(3,5-difluorophenyl)isoxazolidine-2-carbonyl)cyclohexyl)methyl)-5-methyl-1H-imidazole-4-carbonitrile,
(92) trans-(4-((5-fluoro-1H-benzo[d]imidazol-1-yl)methyl)cyclohexyl)((S)-3-(pyrazin-2-yl)isoxazolidin-2-yl)methanone,
(93) trans-3-fluoro-5-((S)-2-(4-((6-fluoro-1H-pyrrolo[3,2-b]pyridin-1-yl)methyl)cyclohexane-1-carbonyl)isoxazolidin-3-yl)benzonitrile,
(94) trans-((S)-3-(3,5-difluorophenyl)isoxazolidin-2-yl)(4-((6-fluoro-1H-pyrrolo[3,2-b]pyridin-1-yl)methyl)cyclohexyl)methanone,
(95) trans-3-fluoro-5-((S)-2-(4-((5-fluoro-1H-indol-1-yl)methyl)cyclohexane-1-carbonyl)isoxazolidin-3-yl)benzonitrile,
(96) trans-((S)-3-(3,5-difluorophenyl)isoxazolidin-2-yl)(4-((5-fluoro-1H-indol-1-yl)methyl)cyclohexyl)methanone,
(97) trans-6-fluoro-1-((4-((S)-3-(pyrazin-2-yl)isoxazolidine-2-carbonyl)cyclohexyl)methyl)-1H-benzo[d]imidazole-5-carbonitrile,
(98) trans-5-fluoro-1-((4-((S)-3-(pyrazin-2-yl)isoxazolidine-2-carbonyl)cyclohexyl)methyl)-1H-benzo[d]imidazole-6-carbonitrile,
(99) trans-1-[[4-[(3S)-3-(3-cyano-5-fluoro-phenyl)isoxazolidine-2-carbonyl]cyclohexyl]methyl]pyrrolo[3,2-b]pyridine-5-carbonitrile,
(100) trans-1-((4-((S)-3-(3-cyano-5-fluorophenyl)isoxazolidine-2-carbonyl)cyclohexyl)methyl)-4-fluoro-3-methyl-1H-indazole-6-carbonitrile,
(101) trans-1-((4-((S)-3-(3,5-difluorophenyl)isoxazolidine-2-carbonyl)cyclohexyl)methyl)-4-fluoro-3-methyl-1H-indazole-6-carbonitrile,
(102) trans-1-((4-((S)-3-(3-cyano-5-fluorophenyl)isoxazolidine-2-carbonyl)cyclohexyl)methyl)-4-fluoro-3-methyl-1H-indazole-6-carboxamide,
(103) trans-1-((4-((S)-3-(3,5-difluorophenyl)isoxazolidine-2-carbonyl)cyclohexyl)methyl)-4-fluoro-3-methyl-1H-indazole-6-carboxamide,
(104) trans-1-((4-((S)-3-(3-cyano-5-fluorophenyl)isoxazolidine-2-carbonyl)cyclohexyl)methyl)-1H-indole-6-carboxamide,
(105) trans-1-((4-((S)-3-(3,5-difluorophenyl)isoxazolidine-2-carbonyl)cyclohexyl)methyl)-1H-indole-6-carboxamide,
(106) trans-5-((S)-2-(4-((6-fluoro-1H-benzo[d]imidazol-1-yl)methyl)cyclohexane-1-carbonyl)isoxazolidin-3-yl)nicotinonitrile,
(107) trans-5-((S)-2-(4-((5-fluoro-1H-benzo[d]imidazol-1-yl)methyl)cyclohexane-1-carbonyl)isoxazolidin-3-yl)nicotinonitrile,
(108) trans-1-((4-((S)-3-(5-cyanopyridin-3-yl)isoxazolidine-2-carbonyl)cyclohexyl)methyl)-1H-pyrrolo[3,2-b]pyridine-6-carbonitrile,
(109) trans-5-((4-((S)-3-(3-cyano-5-fluorophenyl)isoxazolidine-2-carbonyl)cyclohexyl)methyl)-2-methylbenzonitrile,
(110) trans-1-((4-((S)-3-(3-cyano-5-fluorophenyl)isoxazolidine-2-carbonyl)cyclohexyl)methyl)-3-methyl-1H-indole-6-carboxamide,
(111) trans-1-((4-((S)-3-(3,5-difluorophenyl)isoxazolidine-2-carbonyl)cyclohexyl)methyl)-3-methyl-1H-indole-6-carboxamide,
(112) trans-3-fluoro-5-((S)-2-(4-((6-fluoro-1H-benzo[d]imidazol-1-yl)methyl)cyclohexane-1-carbonyl)isoxazolidin-3-yl)benzonitrile,
(113) trans-((S)-3-(3,5-difluorophenyl)isoxazolidin-2-yl)(4-((6-fluoro-1H-benzo[d]imidazol-1-yl)methyl)cyclohexyl)methanone,
(114) trans-(3-((5-fluoro-1H-benzo[d]imidazol-1-yl)methyl)cyclobutyl)((S)-3-(p-tolyl)isoxazolidin-2-yl)methanone,
(115) trans-((S)-3-(3,5-difluorophenyl)isoxazolidin-2-yl)(3-((5-fluoro-1H-benzo[d]imidazol-1-yl)methyl)cyclobutyl)methanone,
(116) trans-3-fluoro-5-((S)-2-(3-((5-fluoro-1H-benzo[d]imidazol-1-yl)methyl)cyclobutane-1-carbonyl)isoxazolidin-3-yl)benzonitrile,
(117) trans-(4-((3,5-dimethyl-1H-pyrazol-1-yl)methyl)cyclohexyl)((S)-3-(p-tolyl)isoxazolidin-2-yl)methanone,
(118) trans-((S)-3-(3,5-difluorophenyl)isoxazolidin-2-yl)(4-((3,5-dimethyl-1H-pyrazol-1-yl)methyl)cyclohexyl)methanone,
(119) trans-3-((S)-2-(4-((3,5-dimethyl-1H-pyrazol-1-yl)methyl)cyclohexane-1-carbonyl)isoxazolidin-3-yl)-5-fluorobenzonitrile,
(120) trans-3-fluoro-5-[(3S)-2-[4-[(5-methoxypyrrolo[3,2-b]pyridin-1-yl)methyl]cyclohexanecarbonyl]isoxazolidin-3-yl]benzonitrile,
(121) trans-2-((4-((S)-3-(5-cyanopyridin-3-yl)isoxazolidine-2-carbonyl)cyclohexyl)methyl)-2H-pyrazolo[3,4-b]pyridine-5-carbonitrile,
(122) trans-1-[[4-[(3S)-3-(5-cyano-3-pyridyl)isoxazolidine-2-carbonyl]cyclohexyl]methyl]-6-oxo-pyridine-3-carbonitrile,
(123) trans-5-((S)-2-(4-((6-fluoro-1H-pyrrolo[3,2-b]pyridin-1-yl)methyl)cyclohexane-1-carbonyl)isoxazolidin-3-yl)nicotinonitrile,
(124) trans-3-[(3S)-2-[4-[[6-(3,5-dimethylpyrazol-1-yl)pyrimidin-4-yl]methyl]cyclohexanecarbonyl]isoxazolidin-3-yl]-5-fluoro-benzonitrile,
(125) trans-[(3S)-3-(3,5-difluorophenyl)isoxazolidin-2-yl]-[4-[[6-(3,5-dimethylpyrazol-1-yl)pyrimidin-4-yl]methyl]cyclohexyl]methanone,
(126) trans-5-((S)-2-(4-((5-fluoro-1H-indazol-1-yl)methyl)cyclohexane-1-carbonyl)isoxazolidin-3-yl)nicotinonitrile,
(127) trans-2-((4-((S)-3-(5-cyanopyridin-3-yl)isoxazolidine-2-carbonyl)cyclohexyl)methyl)-2H-indazole-6-carbonitrile,
(128) trans-3-fluoro-5-[(3S)-2-[4-[(6-methoxypyrrolo[3,2-b]pyridin-1-yl)methyl]cyclohexanecarbonyl]isoxazolidin-3-yl]benzonitrile,
(129) trans-3-fluoro-5-[(3S)-2-[4-[(6-hydroxypyrrolo[3,2-b]pyridin-1-yl)methyl]cyclohexanecarbonyl]isoxazolidin-3-yl]benzonitrile,
(130) trans-1-((4-((S)-3-(5-cyanopyridin-3-yl)isoxazolidine-2-carbonyl)cyclohexyl)methyl)-4-fluoro-1H-indole-6-carbonitrile,
(131) trans-2-methyl-5-[[4-[(3S)-3-(p-tolyl)isoxazolidine-2-carbonyl]cyclohexyl]methyl]benzamide,
(132) trans-5-[[4-[(3S)-3-(3,5-difluorophenyl)isoxazolidine-2-carbonyl]cyclohexyl]methyl]-2-methyl-benzamide,
(133) trans-5-[[4-[(3S)-3-(3-cyano-5-fluoro-phenyl)isoxazolidine-2-carbonyl]cyclohexyl]methyl]-2-methyl-benzamide,
(134) trans-1-[[4-[(3S)-3-pyrazin-2-ylisoxazolidine-2-carbonyl]cyclohexyl]methyl]pyrrolo[3,2-b]pyridine-6-carbonitrile,
(135) trans-3-fluoro-5-[(3S)-2-[4-[[6-(2-methylimidazol-1-yl)pyrimidin-4-yl]methyl]cyclohexanecarbonyl]isoxazolidin-3-yl]benzonitrile,
(136) trans-[4-[[6-(2-methylimidazol-1-yl)pyrimidin-4-yl]methyl]cyclohexyl]-[(3S)-3-(p-tolyl)isoxazolidin-2-yl]methanone,
(137) trans-1-[[4-[(3S)-3-pyrazin-2-ylisoxazolidine-2-carbonyl]cyclohexyl]methyl]pyrazolo[4,3-b]pyridine-6-carbonitrile,
(138) trans-2-methyl-5-[[4-[(3S)-3-pyrazin-2-ylisoxazolidine-2-carbonyl]cyclohexyl]methyl]benzamide,
(139) trans-2-methyl-5-[[4-[(3S)-3-pyrazin-2-ylisoxazolidine-2-carbonyl]cyclohexyl]methyl]benzonitrile,
(140) trans-5-[[4-[(3S)-3-pyrazin-2-ylisoxazolidine-2-carbonyl]cyclohexyl]methyl]pyridine-3-carbonitrile,
(141) trans-5-[[4-[(3S)-3-(3-cyano-5-fluoro-phenyl)isoxazolidine-2-carbonyl]cyclohexyl]methyl]pyridine-3-carbonitrile,
(142) trans-3-fluoro-5-[(3S)-2-[4-([1,2,4]triazolo[1,5-a]pyridin-6-ylmethyl)cyclohexanecarbonyl]isoxazolidin-3-yl]benzonitrile,
(143) trans-[(3S)-3-pyrazin-2-ylisoxazolidin-2-yl]-[4-([1,2,4]triazolo[1,5-a]pyridin-6-ylmethyl)cyclohexyl]methanone,
(144) trans-3-fluoro-5-[(3S)-2-[4-[(3-oxoisoindolin-5-yl)methyl]cyclohexanecarbonyl]isoxazolidin-3-yl]benzonitrile,
(145) trans-6-[[4-[(3S)-3-pyrazin-2-ylisoxazolidine-2-carbonyl]cyclohexyl]methyl]isoindolin-1-one,
(146) trans-3-fluoro-5-[(3S)-2-[4-[[6-(oxetan-3-yloxy)pyrrolo[3,2-b]pyridin-1-yl]methyl]cyclohexanecarbonyl]isoxazolidin-3-yl]benzonitrile,
(147) trans-[4-[(5-fluoro-3-pyridyl)methyl]cyclohexyl]-[(3S)-3-pyrazin-2-ylisoxazolidin-2-yl]methanone,
(148) trans-3-fluoro-5-[(3S)-2-[4-[(5-fluoro-3-pyridyl)methyl]cyclohexanecarbonyl]isoxazolidin-3-yl]benzonitrile,
(149) trans-3-fluoro-5-[(3S)-2-[4-[[6-(2-methoxyethoxy)pyrrolo[3,2-b]pyridin-1-yl]methyl]cyclohexanecarbonyl]isoxazolidin-3-yl]benzonitrile,
(150) trans-3-fluoro-5-[(3S)-2-[4-[(1-methylpyrazol-4-yl)methyl]cyclohexanecarbonyl]isoxazolidin-3-yl]benzonitrile,
(151) trans-[4-[(1-methylpyrazol-4-yl)methyl]cyclohexyl]-[(3S)-3-pyrazin-2-ylisoxazolidin-2-yl]methanone,
(152) trans-3-[(3S)-2-[4-[[6-(cyclopropylmethoxy)pyrrolo[3,2-b]pyridin-1-yl]methyl]cyclohexanecarbonyl]isoxazolidin-3-yl]-5-fluoro-benzonitrile,
(153) trans-3-fluoro-5-[(3S)-2-[4-[[6-(2-hydroxyethoxy)pyrrolo[3,2-b]pyridin-1-yl]methyl]cyclohexanecarbonyl]isoxazolidin-3-yl]benzonitrile,
(154) trans-6-[[4-[(3S)-3-(4-fluorophenyl)isoxazolidine-2-carbonyl]cyclohexyl]methyl]isoindolin-1-one,
(155) trans-6-[[4-[(3S)-3-(5-fluoro-3-pyridyl)isoxazolidine-2-carbonyl]cyclohexyl]methyl]isoindolin-1-one,
(156) trans-6-[[4-[(3S)-3-(3,5-difluorophenyl)isoxazolidine-2-carbonyl]cyclohexyl]methyl]isoindolin-1-one,
(157) trans-6-[[4-[(3S)-3-(6-methyl-3-pyridyl)isoxazolidine-2-carbonyl]cyclohexyl]methyl]isoindolin-1-one,
(158) trans-5-[[4-[(3S)-3-(5-cyano-3-pyridyl)isoxazolidine-2-carbonyl]cyclohexyl]methyl]-2-methyl-benzamide,
(159) trans-2-methyl-5-[[4-[(3S)-3-(2-methylthiazol-4-yl)isoxazolidine-2-carbonyl]cyclohexyl]methyl]benzamide,
(160) trans-3-fluoro-5-[[4-[(3S)-3-(5-fluoro-3-pyridyl)isoxazolidine-2-carbonyl]cyclohexyl]methyl]-N-methyl-benzamide,
(161) trans-3-[[4-[(3S)-3-(3,5-difluorophenyl)isoxazolidine-2-carbonyl]cyclohexyl]methyl]-5-fluoro-N-methyl-benzamide,
(162) trans-3-fluoro-N-methyl-5-[[4-[(3S)-3-pyrazin-2-ylisoxazolidine-2-carbonyl]cyclohexyl]methyl]benzamide,
(163) trans-3-[[4-[(3S)-3-(5-cyano-3-pyridyl)isoxazolidine-2-carbonyl]cyclohexyl]methyl]-5-fluoro-N-methyl-benzamide,
(164) trans-3-[[4-[(3S)-3-(3-cyano-5-fluoro-phenyl)isoxazolidine-2-carbonyl]cyclohexyl]methyl]-5-fluoro-N-methyl-benzamide,
(165) trans-3-fluoro-5-[[4-[(3S)-3-(4-fluorophenyl)isoxazolidine-2-carbonyl]cyclohexyl]methyl]-N-methyl-benzamide,
(166) trans-5-[[4-[(3S)-3-(6-methoxypyrazin-2-yl)isoxazolidine-2-carbonyl]cyclohexyl]methyl]-2-methyl-benzamide,
(167) trans-3-fluoro-5-[(3S)-2-[4-[(3-methylimidazol-4-yl)methyl]cyclohexanecarbonyl]isoxazolidin-3-yl]benzonitrile,
(168) trans-5-[(3S)-2-[4-[(3-methylimidazol-4-yl)methyl]cyclohexanecarbonyl]isoxazolidin-3-yl]pyridine-3-carbonitrile,
(169) trans-3-fluoro-5-[(3S)-2-[4-[[4-(3-hydroxyoxetan-3-yl)phenyl]methyl]cyclohexanecarbonyl]isoxazolidin-3-yl]benzonitrile,
(170) trans-[(3S)-3-(4-fluorophenyl)isoxazolidin-2-yl]-[4-[[4-(3-hydroxyoxetan-3-yl)phenyl]methyl]cyclohexyl]methanone,
(171) trans-[(3S)-3-(3,5-difluorophenyl)isoxazolidin-2-yl]-[4-[[4-(3-hydroxyoxetan-3-yl)phenyl]methyl]cyclohexyl]methanone,
(172) trans-4-fluoro-3-[[4-[(3S)-3-(5-fluoro-3-pyridyl)isoxazolidine-2-carbonyl]cyclohexyl]methyl]-N-methyl-benzamide,
(173) trans-4-fluoro-3-[[4-[(3S)-3-(4-fluorophenyl)isoxazolidine-2-carbonyl]cyclohexyl]methyl]-N-methyl-benzamide,
(174) trans-3-[[4-[(3S)-3-(3,5-difluorophenyl)isoxazolidine-2-carbonyl]cyclohexyl]methyl]-4-fluoro-N-methyl-benzamide,
(175) trans-4-fluoro-N-methyl-3-[[4-[(3S)-3-pyrazin-2-ylisoxazolidine-2-carbonyl]cyclohexyl]methyl]benzamide,
(176) trans-3-[[4-[(3S)-3-(5-cyano-3-pyridyl)isoxazolidine-2-carbonyl]cyclohexyl]methyl]-4-fluoro-N-methyl-benzamide,
(177) trans-3-[[4-[(3S)-3-(3-cyano-5-fluoro-phenyl)isoxazolidine-2-carbonyl]cyclohexyl]methyl]-4-fluoro-N-methyl-benzamide,
(178) trans-[4-[(5-fluoro-2-methyl-3-pyridyl)methyl]cyclohexyl]-[(3S)-3-(5-fluoro-3-pyridyl)isoxazolidin-2-yl]methanone,
(179) trans-[4-[(5-fluoro-2-methyl-3-pyridyl)methyl]cyclohexyl]-[(3S)-3-(4-fluorophenyl)isoxazolidin-2-yl]methanone,
(180) trans-[4-[(5-fluoro-2-methyl-3-pyridyl)methyl]cyclohexyl]-[(3S)-3-pyrazin-2-ylisoxazolidin-2-yl]methanone,
(181) trans-3-fluoro-5-[(3S)-2-[4-[(5-fluoro-2-methyl-3-pyridyl)methyl]cyclohexanecarbonyl]isoxazolidin-3-yl]benzonitrile,
(182) trans-[(3S)-3-(3,5-difluorophenyl)isoxazolidin-2-yl]-[4-[(5-fluoro-2-methyl-3-pyridyl)methyl]cyclohexyl]methanone,
(183) trans-5-[(3S)-2-[4-[[4-(3-hydroxyoxetan-3-yl)phenyl]methyl]cyclohexanecarbonyl]isoxazolidin-3-yl]pyridine-3-carbonitrile,
(184) trans-3-fluoro-5-[(3S)-2-[4-[[6-(1-methylcyclopropoxy)pyrrolo[3,2-b]pyridin-1-yl]methyl]cyclohexanecarbonyl]isoxazolidin-3-yl]benzonitrile,
(185) trans-1-[[4-[(3S)-3-(6-methoxypyrazin-2-yl)isoxazolidine-2-carbonyl]cyclohexyl]methyl]pyrrolo[3,2-b]pyridine-6-carbonitrile,
(186) trans-1-[[4-[(3S)-3-(6-methoxypyrazin-2-yl)isoxazolidine-2-carbonyl]cyclohexyl]methyl]pyrazolo[4,3-b]pyridine-6-carbonitrile,
(187) trans-1-[[4-[(3S)-3-(2-pyridyl)isoxazolidine-2-carbonyl]cyclohexyl]methyl]pyrazolo[4,3-b]pyridine-6-carbonitrile,
(188) trans-1-[[4-[(3S)-3-(2-pyridyl)isoxazolidine-2-carbonyl]cyclohexyl]methyl]pyrrolo[3,2-b]pyridine-6-carbonitrile,
(189) trans-2-[[4-[(3S)-3-(6-methoxypyrazin-2-yl)isoxazolidine-2-carbonyl]cyclohexyl]methyl]indazole-6-carbonitrile,
(190) trans-1-[[4-[(3S)-3-(6-methoxypyrazin-2-yl)isoxazolidine-2-carbonyl]cyclohexyl]methyl]indazole-6-carbonitrile,
(191) trans-2-fluoro-5-[[4-[(3S)-3-(4-fluorophenyl)isoxazolidine-2-carbonyl]cyclohexyl]methyl]benzamide,
(192) trans-5-[[4-[(3S)-3-(3,5-difluorophenyl)isoxazolidine-2-carbonyl]cyclohexyl]methyl]-2-fluoro-benzamide,
(193) trans-2-fluoro-5-[[4-[(3S)-3-pyrazin-2-ylisoxazolidine-2-carbonyl]cyclohexyl]methyl]benzamide,
(194) trans-5-[[4-[(3S)-3-(5-cyano-3-pyridyl)isoxazolidine-2-carbonyl]cyclohexyl]methyl]-2-fluoro-benzamide,
(195) trans-5-[[4-[(3S)-3-(3-cyano-5-fluoro-phenyl)isoxazolidine-2-carbonyl]cyclohexyl]methyl]-2-fluoro-benzamide,
(196) trans-2-[[4-[(3S)-3-pyrazin-2-ylisoxazolidine-2-carbonyl]cyclohexyl]methyl]indazole-6-carbonitrile,
(197) trans-5-[(3S)-2-[4-[(5-fluoro-2-methyl-3-pyridyl)methyl]cyclohexanecarbonyl]isoxazolidin-3-yl]pyridine-3-carbonitrile,
(198) trans-1-[[4-[(3S)-3-(2-methylthiazol-4-yl)isoxazolidine-2-carbonyl]cyclohexyl]methyl]pyrrolo[3,2-b]pyridine-6-carbonitrile,
(199) trans-1-[[4-[(3S)-3-(2-methylthiazol-4-yl)isoxazolidine-2-carbonyl]cyclohexyl]methyl]pyrazolo[4,3-b]pyridine-6-carbonitrile,
(200) trans-1-[[4-[(3S)-3-pyrazin-2-ylisoxazolidine-2-carbonyl]cyclohexyl]methyl]indazole-6-carbonitrile,
(201) trans-1-[[4-[(3S)-3-(6-methoxypyrazin-2-yl)isoxazolidine-2-carbonyl]cyclohexyl]methyl]indazole-6-carboxamide,
(202) trans-1-[[4-[(3S)-3-(2-methylthiazol-4-yl)isoxazolidine-2-carbonyl]cyclohexyl]methyl]indazole-5-carbonitrile,
(203) trans-2-[[4-[(3S)-3-(2-methylthiazol-4-yl)isoxazolidine-2-carbonyl]cyclohexyl]methyl]indazole-6-carbonitrile,
(204) trans-1-[[4-[(3S)-3-(2-methylthiazol-4-yl)isoxazolidine-2-carbonyl]cyclohexyl]methyl]indazole-6-carbonitrile,
(205) trans-[4-[(8-fluoro-[1,2,4]triazolo[1,5-a]pyridin-6-yl)methyl]cyclohexyl]-[(3S)-3-pyrazin-2-ylisoxazolidin-2-yl]methanone,
(206) trans-3-fluoro-5-[(3S)-2-[4-[(8-fluoro-[1,2,4]triazolo[1,5-a]pyridin-6-yl)methyl]cyclohexanecarbonyl]isoxazolidin-3-yl]benzonitrile,
(207) trans-[4-[[3-fluoro-5-(2-methylimidazol-1-yl)phenyl]methyl]cyclohexyl]-[(3S)-3-(4-fluorophenyl)isoxazolidin-2-yl]methanone,
(208) trans-[(3S)-3-(3,5-difluorophenyl)isoxazolidin-2-yl]-[4-[[3-fluoro-5-(2-methylimidazol-1-yl)phenyl]methyl]cyclohexyl]methanone,
(209) trans-[4-[[3-fluoro-5-(2-methylimidazol-1-yl)phenyl]methyl]cyclohexyl]-[(3S)-3-pyrazin-2-ylisoxazolidin-2-yl]methanone,
(210) trans-5-[(3S)-2-[4-[[3-fluoro-5-(2-methylimidazol-1-yl)phenyl]methyl]cyclohexanecarbonyl]isoxazolidin-3-yl]pyridine-3-carbonitrile,
(211) trans-[4-[[3-fluoro-5-(2-methylpyrazol-3-yl)phenyl]methyl]cyclohexyl]-[(3S)-3-pyrazin-2-ylisoxazolidin-2-yl]methanone,
(212) trans-4-fluoro-6-[[4-[(3S)-3-(4-fluorophenyl)isoxazolidine-2-carbonyl]cyclohexyl]methyl]isoindolin-1-one,
(213) trans-4-fluoro-6-[[4-[(3S)-3-pyrazin-2-ylisoxazolidine-2-carbonyl]cyclohexyl]methyl]isoindolin-1-one,
(214) trans-3-fluoro-5-[(3S)-2-[4-[[3-fluoro-5-(2-methylpyrazol-3-yl)phenyl]methyl]cyclohexanecarbonyl]isoxazolidin-3-yl]benzonitrile,
(215) trans-1-[[4-[(3S)-3-(2-pyridyl)isoxazolidine-2-carbonyl]cyclohexyl]methyl]indazole-6-carbonitrile,
(216) trans-3-fluoro-5-[(3S)-2-[4-[[3-fluoro-5-(2-methylimidazol-1-yl)phenyl]methyl]cyclohexanecarbonyl]isoxazolidin-3-yl]benzonitrile,
(217) trans-1-[[4-[(3S)-3-(6-methoxypyrazin-2-yl)isoxazolidine-2-carbonyl]cyclohexyl]methyl]indazole-5-carbonitrile,
(218) trans-1-[[4-[(3S)-3-pyrazin-2-ylisoxazolidine-2-carbonyl]cyclohexyl]methyl]indazole-5-carbonitrile,
(219) trans-3-fluoro-5-[(3S)-2-[4-[(7-fluoro-3-oxo-isoindolin-5-yl)methyl]cyclohexanecarbonyl]isoxazolidin-3-yl]benzonitrile,
(220) trans-[4-[[3-fluoro-5-(2-methylimidazol-1-yl)phenyl]methyl]cyclohexyl]-[(3S)-3-(5-fluoro-3-pyridyl)isoxazolidin-2-yl]methanone,
(221) trans-3-fluoro-5-[(3S)-2-[4-[(2-methyl-[1,2,4]triazolo[1,5-a]pyridin-6-yl)methyl]cyclohexanecarbonyl]isoxazolidin-3-yl]benzonitrile,
(222) trans-[4-[(2-methyl-[1,2,4]triazolo[1,5-a]pyridin-6-yl)methyl]cyclohexyl]-[(3S)-3-pyrazin-2-ylisoxazolidin-2-yl]methanone,
(223) trans-[(3S)-3-(3,5-difluorophenyl)isoxazolidin-2-yl]-[4-[(2-methyl-[1,2,4]triazolo[1,5-a]pyridin-6-yl)methyl]cyclohexyl]methanone,
(224) trans-3-fluoro-5-[(3S)-2-[4-[(3-methyl-[1,2,4]triazolo[4,3-a]pyridin-6-yl)methyl]cyclohexanecarbonyl]isoxazolidin-3-yl]benzonitrile,
(225) trans-3-fluoro-5-[(3S)-2-[4-[(2-methylimidazo[1,2-a]pyridin-6-yl)methyl]cyclohexanecarbonyl]isoxazolidin-3-yl]benzonitrile,
(226) trans-[4-[(2-methylimidazo[1,2-a]pyridin-6-yl)methyl]cyclohexyl]-[(3S)-3-pyrazin-2-ylisoxazolidin-2-yl]methanone,
(227) trans-[(3S)-3-(3,5-difluorophenyl)isoxazolidin-2-yl]-[4-[(2-methylimidazo[1,2-a]pyridin-6-yl)methyl]cyclohexyl]methanone,
(228) trans-2-[[4-[(3S)-3-pyrazin-2-ylisoxazolidine-2-carbonyl]cyclohexyl]methyl]indazole-6-carboxamide,
(229) trans-1-[[4-[(3S)-3-pyrazin-2-ylisoxazolidine-2-carbonyl]cyclohexyl]methyl]indazole-6-carboxamide,
(230) trans-1-[[4-[(3S)-3-(2-pyridyl)isoxazolidine-2-carbonyl]cyclohexyl]methyl]indazole-6-carboxamide,
(231) trans-1-[[4-[(3S)-3-(4-cyano-2-pyridyl)isoxazolidine-2-carbonyl]cyclohexyl]methyl]indazole-6-carboxamide,
(232) trans-2-[[4-[(3S)-3-(4-cyano-2-pyridyl)isoxazolidine-2-carbonyl]cyclohexyl]methyl]indazole-6-carboxamide,
(233) trans-2-[[4-[(3S)-3-(2-methylthiazol-4-yl)isoxazolidine-2-carbonyl]cyclohexyl]methyl]indazole-6-carboxamide,
(234) trans-1-[[4-[(3S)-3-(2-methylthiazol-4-yl)isoxazolidine-2-carbonyl]cyclohexyl]methyl]indazole-6-carboxamide,
(235) trans-5-[(3S)-2-[4-[(1-methylpyrazol-4-yl)methyl]cyclohexanecarbonyl]isoxazolidin-3-yl]pyridine-3-carbonitrile,
(236) trans-[(3S)-3-(3,5-difluorophenyl)isoxazolidin-2-yl]-[4-[(1-methylpyrazol-4-yl)methyl]cyclohexyl]methanone,
(237) trans-3-fluoro-5-[(3S)-2-[4-[(2-methylpyrazol-3-yl)methyl]cyclohexanecarbonyl]isoxazolidin-3-yl]benzonitrile,
(238) trans-[4-[(2-methylpyrazol-3-yl)methyl]cyclohexyl]-[(3S)-3-pyrazin-2-ylisoxazolidin-2-yl]methanone,
(239) trans-3-fluoro-5-[(3S)-2-[4-[(2-methylthiazol-4-yl)methyl]cyclohexanecarbonyl]isoxazolidin-3-yl]benzonitrile,
(240) trans-[4-[(2-methylthiazol-4-yl)methyl]cyclohexyl]-[(3S)-3-pyrazin-2-ylisoxazolidin-2-yl]methanone,
(241) trans-1-[[4-[(3S)-3-(6-methoxypyrazin-2-yl)isoxazolidine-2-carbonyl]cyclohexyl]methyl]indole-6-carbonitrile,
(242) trans-2-fluoro-5-[[4-[(3S)-3-(4-fluorophenyl)isoxazolidine-2-carbonyl]cyclohexyl]methyl]benzonitrile,
(243) trans-2-fluoro-5-[[4-[(3S)-3-pyrazin-2-ylisoxazolidine-2-carbonyl]cyclohexyl]methyl]benzonitrile,
(244) trans-5-[[4-[(3S)-3-(3-cyano-5-fluoro-phenyl)isoxazolidine-2-carbonyl]cyclohexyl]methyl]-2-fluoro-benzonitrile,
(245) trans-3-fluoro-5-[(3S)-2-[4-(pyrazin-2-ylmethyl)cyclohexanecarbonyl]isoxazolidin-3-yl]benzonitrile,
(246) trans-[(3S)-3-pyrazin-2-ylisoxazolidin-2-yl]-[4-(pyrazin-2-ylmethyl)cyclohexyl]methanone,
(247) trans-3-fluoro-5-[(3S)-2-[4-(pyrimidin-5-ylmethyl)cyclohexanecarbonyl]isoxazolidin-3-yl]benzonitrile,
(248) trans-[(3S)-3-pyrazin-2-ylisoxazolidin-2-yl]-[4-(pyrimidin-5-ylmethyl)cyclohexyl]methanone,
(249) trans-3-fluoro-5-[(3S)-2-[4-[(8-methyl-[1,2,4]triazolo[1,5-a]pyridin-6-yl)methyl]cyclohexanecarbonyl]-1,2-oxazolidin-3-yl]benzonitrile,
(250) trans-[4-[(8-methyl-[1,2,4]triazolo[1,5-a]pyridin-6-yl)methyl]cyclohexyl]-[(3S)-3-pyrazin-2-yl-1,2-oxazolidin-2-yl]methanone,
(251) trans-1-[[4-[(3S)-3-pyrazin-2-ylisoxazolidine-2-carbonyl]cyclohexyl]methyl]indole-5-carboxamide,
(252) trans-1-[[4-[(3S)-3-(5-cyano-3-pyridyl)isoxazolidine-2-carbonyl]cyclohexyl]methyl]indole-5-carboxamide,
(253) trans-1-[[4-[(3S)-3-(6-methoxypyrazin-2-yl)isoxazolidine-2-carbonyl]cyclohexyl]methyl]indole-5-carboxamide,
(254) trans-1-[[4-[(3S)-3-(2-methylthiazol-4-yl)isoxazolidine-2-carbonyl]cyclohexyl]methyl]indole-5-carboxamide,
(255) trans-1-[[4-[(3S)-3-(2-pyridyl)isoxazolidine-2-carbonyl]cyclohexyl]methyl]indole-5-carboxamide,
(256) trans-3-fluoro-5-[(3S)-2-[4-[[3-fluoro-5-(hydroxymethyl)phenyl]methyl]cyclohexanecarbonyl]isoxazolidin-3-yl]benzonitrile,
(257) trans-5-[(3S)-2-[4-[[3-fluoro-5-(hydroxymethyl)phenyl]methyl]cyclohexanecarbonyl]isoxazolidin-3-yl]pyridine-3-carbonitrile,
(258) trans-3-fluoro-5-[(3S)-2-[4-[(1-methyltriazol-4-yl)methyl]cyclohexanecarbonyl]isoxazolidin-3-yl]benzonitrile,
(259) trans-[4-[[3-fluoro-5-(hydroxymethyl)phenyl]methyl]cyclohexyl]-[(3S)-3-pyrazin-2-ylisoxazolidin-2-yl]methanone,
(260) trans-[(3S)-3-(4-fluorophenyl)isoxazolidin-2-yl]-[4-[(2-methyl-[1,2,4]triazolo[1,5-a]pyridin-6-yl)methyl]cyclohexyl]methanone,
(261) trans-[(3S)-3-(3,4-difluorophenyl)isoxazolidin-2-yl]-[4-[(2-methyl-[1,2,4]triazolo[1,5-a]pyridin-6-yl)methyl]cyclohexyl]methanone,
(262) trans-5-[(3S)-2-[4-[(2-methyl-[1,2,4]triazolo[1,5-a]pyridin-6-yl)methyl]cyclohexanecarbonyl]isoxazolidin-3-yl]pyridine-3-carbonitrile,
(263) trans-3-fluoro-5-[(3S)-2-[4-[(2-methylpyrimidin-5-yl)methyl]cyclohexanecarbonyl]isoxazolidin-3-yl]benzonitrile,
(264) trans-[(3S)-3-(3,5-difluorophenyl)isoxazolidin-2-yl]-[4-[(2-methylpyrimidin-5-yl)methyl]cyclohexyl]methanone,
(265) trans-[(3S)-3-(3,5-difluorophenyl)isoxazolidin-2-yl]-[4-[[3-fluoro-4-(hydroxymethyl)phenyl]methyl]cyclohexyl]methanone,
(266) trans-3-fluoro-5-[(3S)-2-[4-[[3-fluoro-4-(hydroxymethyl)phenyl]methyl]cyclohexanecarbonyl]isoxazolidin-3-yl]benzonitrile,
(267) trans-5-[[4-[(3S)-3-(3,5-difluorophenyl)isoxazolidine-2-carbonyl]cyclohexyl]methyl]-2-methyl-pyridine-3-carbonitrile,
(268) trans-5-[[4-[(3S)-3-(3-cyano-5-fluoro-phenyl)isoxazolidine-2-carbonyl]cyclohexyl]methyl]-2-methyl-pyridine-3-carbonitrile,
(269) 5-[[4-[(3S)-3-(3,5-difluorophenyl)isoxazolidine-2-carbonyl]norbornan-1-yl]methyl]-2-methyl-benzamide,
(270) 5-[[4-[(3S)-3-(3-cyano-5-fluoro-phenyl)isoxazolidine-2-carbonyl]norbornan-1-yl]methyl]-2-methyl-benzamide,
(271) 2-methyl-5-[[4-[(3S)-3-(6-methyl-3-pyridyl)isoxazolidine-2-carbonyl]norbornan-1-yl]methyl]benzamide,
(272) 2-methyl-5-[[4-[(3S)-3-(6-methyl-3-pyridyl)isoxazolidine-2-carbonyl]-1-bicyclo[2.2.2]octanyl]methyl]benzamide,
(273) 5-[[4-[(3S)-3-(3,5-difluorophenyl)-1,2-oxazolidine-2-carbonyl]-1-bicyclo[2.2.2]octanyl]methyl]-2-methylbenzamide,
(274) 5-[[4-[(3S)-3-(3-cyano-5-fluoro-phenyl)isoxazolidine-2-carbonyl]-1-bicyclo[2.2.2]octanyl]methyl]-2-methyl-benzamide,
(275) trans-5-[[4-[(3S)-3-(3,5-difluorophenyl)isoxazolidine-2-carbonyl]cyclohexyl]methyl]-4-methyl-pyridine-3-carbonitrile,
(276) trans-5-[[4-[(3S)-3-(3-cyano-5-fluoro-phenyl)isoxazolidine-2-carbonyl]cyclohexyl]methyl]-4-methyl-pyridine-3-carbonitrile,
(277) trans-5-[[4-[(3S)-3-(3-cyano-5-fluoro-phenyl)isoxazolidine-2-carbonyl]cyclohexyl]methyl]-6-methyl-pyridine-3-carbonitrile,
(278) trans-5-[[4-[(3S)-3-(3,5-difluorophenyl)isoxazolidine-2-carbonyl]cyclohexyl]methyl]-6-methyl-pyridine-3-carbonitrile formic acid salt,
(279) trans-3-fluoro-5-[(3S)-2-[4-[[3-fluoro-4-(2-hydroxyethyl)phenyl]methyl]cyclohexanecarbonyl]isoxazolidin-3-yl]benzonitrile,
(280) trans-[(3S)-3-(3,5-difluorophenyl)isoxazolidin-2-yl]-[4-[[3-fluoro-4-(2-hydroxyethyl)phenyl]methyl]cyclohexyl]methanone,
(281) trans-[(3S)-3-(3,5-difluorophenyl)isoxazolidin-2-yl]-[4-[(8-fluoro-2-methyl-imidazo[1,2-a]pyridin-6-yl)methyl]cyclohexyl]methanone,
(282) trans-3-fluoro-5-[(3S)-2-[4-[[3-fluoro-5-(2-hydroxyethyl)phenyl]methyl]cyclohexanecarbonyl]isoxazolidin-3-yl]benzonitrile,
(283) trans-[(3S)-3-(3,5-difluorophenyl)isoxazolidin-2-yl]-[4-[[3-fluoro-5-(2-hydroxyethyl)phenyl]methyl]cyclohexyl]methanone,
(284) trans-3-fluoro-5-[(3S)-2-[4-[(8-fluoro-2-methyl-[1,2,4]triazolo[1,5-a]pyridin-6-yl)methyl]cyclohexanecarbonyl]isoxazolidin-3-yl]benzonitrile,
(285) trans-[(3S)-3-(3,5-difluorophenyl)isoxazolidin-2-yl]-[4-[(8-fluoro-2-methyl-[1,2,4]triazolo[1,5-a]pyridin-6-yl)methyl]cyclohexyl]methanone,
(286) trans-5-[[4-[(3S)-3-(3-cyano-5-fluoro-4-methyl-phenyl)isoxazolidine-2-carbonyl]cyclohexyl]methyl]-2-methyl-pyridine-3-carbonitrile,
(287) trans-5-[[4-[(3S)-3-(3-cyano-5-fluoro-4-methyl-phenyl)isoxazolidine-2-carbonyl]cyclohexyl]methyl]-4-methyl-pyridine-3-carbonitrile,
(288) trans-3-[(3S)-2-[4-[(2,8-dimethyl-[1,2,4]triazolo[1,5-a]pyridin-6-yl)methyl]cyclohexanecarbonyl]isoxazolidin-3-yl]-5-fluoro-benzonitrile,
(289) trans-4-fluoro-3-[(3S)-2-[4-[(2-methyl-[1,2,4]triazolo[1,5-a]pyridin-6-yl)methyl]cyclohexanecarbonyl]isoxazolidin-3-yl]benzonitrile,
(290) trans-3-[(3S)-2-[4-[(2,8-dimethylimidazo[1,2-b]pyridazin-6-yl)methyl]cyclohexanecarbonyl]isoxazolidin-3-yl]-5-fluoro-benzonitrile,
(291) trans-[(3S)-3-(3,5-difluorophenyl)isoxazolidin-2-yl]-[4-[(2,8-dimethylimidazo[1,2-b]pyridazin-6-yl)methyl]cyclohexyl]methanone,
(292) trans-3-[(3S)-2-[4-[(2,7-dimethylimidazo[1,2-b]pyridazin-6-yl)methyl]cyclohexanecarbonyl]isoxazolidin-3-yl]-5-fluoro-benzonitrile,
(293) trans-[(3S)-3-(3,5-difluorophenyl)isoxazolidin-2-yl]-[4-[(2,7-dimethylimidazo[1,2-b]pyridazin-6-yl)methyl]cyclohexyl]methanone,
(294) trans-3-fluoro-5-[(3S)-2-[4-[(1-methylindazol-6-yl)methyl]cyclohexanecarbonyl]isoxazolidin-3-yl]benzonitrile,
(295) trans-[(3S)-3-(3,5-difluorophenyl)isoxazolidin-2-yl]-[4-[(1-methylindazol-6-yl)methyl]cyclohexyl]methanone,
(296) trans-3-fluoro-5-[(3S)-2-[4-[(1-methylpyrazolo[4,3-b]pyridin-6-yl)methyl]cyclohexanecarbonyl]isoxazolidin-3-yl]benzonitrile,
(297) trans-[(3S)-3-(3,5-difluorophenyl)isoxazolidin-2-yl]-[4-[(1-methylpyrazolo[4,3-b]pyridin-6-yl)methyl]cyclohexyl]methanone,
(298) 6-fluoro-1-[[trans-4-[(3S)-3-(6-methyl-3-pyridyl)isoxazolidine-2-carbonyl]cyclohexyl]methyl]indazole-5-carbonitrile,
(299) trans-2-fluoro-5-[[4-[(3S)-3-(6-methylpyrazin-2-yl)isoxazolidine-2-carbonyl]cyclohexyl]methyl]benzamide,
(300) trans-3-[(3S)-2-[4-[(3,8-dimethyl-[1,2,4]triazolo[4,3-a]pyridin-6-yl)methyl]cyclohexanecarbonyl]-1,2-oxazolidin-3-yl]-5-fluorobenzonitrile,
(301) trans-[4-[(3,8-dimethyl-[1,2,4]triazolo[4,3-a]pyridin-6-yl)methyl]cyclohexyl]-[(3S)-3-pyrazin-2-yl-1,2-oxazolidin-2-yl]methanone,
(302) trans-3-fluoro-5-[(3S)-2-[4-[(6-methylpyridazin-4-yl)methyl]cyclohexanecarbonyl]isoxazolidin-3-yl]benzonitrile,
(303) trans-3-fluoro-5-[(3S)-2-[4-[(6-methylpyridazin-3-yl)methyl]cyclohexanecarbonyl]isoxazolidin-3-yl]benzonitrile,
(304) trans-[(3S)-3-(3,5-difluorophenyl)isoxazolidin-2-yl]-[4-[(6-methylpyridazin-3-yl)methyl]cyclohexyl]methanone,
(305) trans-[4-[[5-(2,5-dimethylpyrazol-3-yl)-3-pyridyl]methyl]cyclohexyl]-[(3S)-3-pyrazin-2-ylisoxazolidin-2-yl]methanone,
(306) trans-[(3S)-3-(4-fluorophenyl)isoxazolidin-2-yl]-[4-[(2-methylpyrazol-3-yl)methyl]cyclohexyl]methanone,
(307) trans-[(3S)-3-(3,5-difluorophenyl)isoxazolidin-2-yl]-[4-[(2-methylpyrazol-3-yl)methyl]cyclohexyl]methanone,
(308) trans-[4-[[5-(1,3-dimethylpyrazol-4-yl)-3-pyridyl]methyl]cyclohexyl]-[(3S)-3-pyrazin-2-ylisoxazolidin-2-yl]methanone,
(309) trans-[(3S)-3-(3,4-difluorophenyl)isoxazolidin-2-yl]-[4-[(2-methylpyrimidin-5-yl)methyl]cyclohexyl]methanone,
(310) trans-[(3S)-3-(3,4-difluorophenyl)isoxazolidin-2-yl]-[4-[(2-methyl-[1,2,4]triazolo[1,5-b]pyridazin-6-yl)methyl]cyclohexyl]methanone,
(311) trans-[(3S)-3-(3,4-difluorophenyl)isoxazolidin-2-yl]-[4-[(2-methylpyrazol-3-yl)methyl]cyclohexyl]methanone,
(312) trans-3-[(3S)-2-[4-[(2,6-dimethylpyrimidin-4-yl)methyl]cyclohexanecarbonyl]isoxazolidin-3-yl]-5-fluoro-benzonitrile,
(313) trans-[(3S)-3-(3,5-difluorophenyl)isoxazolidin-2-yl]-[4-[(6-methylpyrimidin-4-yl)methyl]cyclohexyl]methanone,
(314) trans-[4-[(8-methoxy-2-methyl-[1,2,4]triazolo[1,5-a]pyridin-6-yl)methyl]cyclohexyl]-[(3S)-3-pyrazin-2-yl-1,2-oxazolidin-2-yl]methanone,
(315) trans-[(3S)-3-(3,5-difluorophenyl)-1,2-oxazolidin-2-yl]-[4-[(2-methyl-[1,2,4]triazolo[1,5-b]pyridazin-6-yl)methyl]cyclohexyl]methanone,
(316) trans-[4-[(5-fluoro-2-methyl-3-pyridyl)methyl]cyclohexyl]-[(3S)-3-(5-methylpyrazin-2-yl)isoxazolidin-2-yl]methanone,
(317) trans-[4-[(3,7-dimethyl-[1,2,4]triazolo[4,3-a]pyridin-6-yl)methyl]cyclohexyl]-[(3S)-3-(3-fluorophenyl)isoxazolidin-2-yl]methanone,
(318) trans-[(3S)-3-(3,4-difluorophenyl)isoxazolidin-2-yl]-[4-[(3,7-dimethyl-[1,2,4]triazolo[4,3-a]pyridin-6-yl)methyl]cyclohexyl]methanone,
(319) trans-[4-[(3,8-dimethyl-[1,2,4]triazolo[4,3-a]pyridin-6-yl)methyl]cyclohexyl]-[(3S)-3-(4-fluorophenyl)isoxazolidin-2-yl]methanone,
(320) trans-[4-[(8-fluoro-3-methyl-[1,2,4]triazolo[4,3-a]pyridin-6-yl)methyl]cyclohexyl]-[(3S)-3-(4-fluorophenyl)isoxazolidin-2-yl]methanone,
(321) trans-[(3S)-3-(4-fluorophenyl)isoxazolidin-2-yl]-[4-[(2-methyl-[1,2,4]triazolo[1,5-a]pyridin-7-yl)methyl]cyclohexyl]methanone,
(322) trans-[2-cyano-4-[(3S)-2-[4-[(2-methylimidazo[1,2-b]pyridazin-7-yl)methyl]cyclohexanecarbonyl]isoxazolidin-3-yl]phenyl],
(323) trans-[2-fluoro-4-[(3S)-2-[4-[(2-methylimidazo[1,2-b]pyridazin-7-yl)methyl]cyclohexanecarbonyl]isoxazolidin-3-yl]phenyl],
(324) trans-[4-[(3S)-2-[4-[(2-methylimidazo[1,2-b]pyridazin-7-yl)methyl]cyclohexanecarbonyl]isoxazolidin-3-yl]phenyl],
(325) trans-[4-[(2,8-dimethyl-[1,2,4]triazolo[1,5-a]pyridin-6-yl)methyl]cyclohexyl]-[(3S)-3-(6-methyl-3-pyridyl)isoxazolidin-2-yl]methanone,
(326) trans-[4-[(2,8-dimethyl-[1,2,4]triazolo[1,5-a]pyridin-6-yl)methyl]cyclohexyl]-[(3S)-3-pyrazin-2-ylisoxazolidin-2-yl]methanone,
(327) trans-[(3S)-3-(5-fluoro-6-methyl-3-pyridyl)isoxazolidin-2-yl]-[4-[(8-fluoro-2-methyl-[1,2,4]triazolo[1,5-a]pyridin-6-yl)methyl]cyclohexyl]methanone,
(328) trans-[4-[(2,8-dimethyl-[1,2,4]triazolo[1,5-a]pyridin-6-yl)methyl]cyclohexyl]-[(3S)-3-(5-methylpyrazin-2-yl)isoxazolidin-2-yl]methanone,
(329) trans-[4-[(1-methylpyrazolo[4,3-b]pyridin-6-yl)methyl]cyclohexyl]-[(3S)-3-(6-methylpyridin-3-yl)-1,2-oxazolidin-2-yl]methanone,
(330) trans-[4-[(2,8-dimethyl-[1,2,4]triazolo[1,5-a]pyridin-6-yl)methyl]cyclohexyl]-[(3S)-3-(5-fluoro-6-methylpyridin-3-yl)-1,2-oxazolidin-2-yl]methanone,
(331) trans-[4-[(2,8-dimethyl-[1,2,4]triazolo[1,5-a]pyridin-6-yl)methyl]cyclohexyl]-[(3S)-3-(5-fluoropyridin-3-yl)-1,2-oxazolidin-2-yl]methanone,
(332) trans-[4-[(2,5-dimethylpyridin-4-yl)methyl]cyclohexyl]-[(3S)-3-(5-fluoro-6-methylpyridin-3-yl)-1,2-oxazolidin-2-yl]methanone,
(333) trans-[4-[(8-fluoro-2-methyl-[1,2,4]triazolo[1,5-a]pyridin-6-yl)methyl]cyclohexyl]-[(3S)-3-(5-methylpyridin-3-yl)-1,2-oxazolidin-2-yl]methanone,
(334) trans-[(3S)-3-(5-methylpyridin-3-yl)-1,2-oxazolidin-2-yl]-[4-[(8-methyl-[1,2,4]triazolo[1,5-a]pyridin-6-yl)methyl]cyclohexyl]methanone,
(335) trans-[(3S)-3-(5-fluoro-6-methylpyridin-3-yl)-1,2-oxazolidin-2-yl]-[4-[(8-methyl-[1,2,4]triazolo[1,5-a]pyridin-6-yl)methyl]cyclohexyl]methanone,
(336) trans-[(3S)-3-(5-fluoropyridin-3-yl)-1,2-oxazolidin-2-yl]-[4-[(8-methyl-[1,2,4]triazolo[1,5-a]pyridin-6-yl)methyl]cyclohexyl]methanone,
(337) trans-[(3S)-3-(5-fluoro-6-methylpyridin-3-yl)-1,2-oxazolidin-2-yl]-[4-([1,2,4]triazolo[1,5-a]pyridin-6-ylmethyl)cyclohexyl]methanone,
(338) trans-[(3S)-3-(5-fluoropyridin-3-yl)-1,2-oxazolidin-2-yl]-[4-([1,2,4]triazolo[1,5-a]pyridin-6-ylmethyl)cyclohexyl]methanone,
(339) trans-[4-[(2,5-dimethylpyridin-4-yl)methyl]cyclohexyl]-[(3S)-3-(5-methylpyrazin-2-yl)-1,2-oxazolidin-2-yl]methanone,
(340) trans-[4-[(8-fluoro-2-methyl-[1,2,4]triazolo[1,5-a]pyridin-6-yl)methyl]cyclohexyl]-[(3S)-3-pyrazin-2-yl-1,2-oxazolidin-2-yl]methanone,
(341) trans-[4-[(8-fluoro-2-methyl-[1,2,4]triazolo[1,5-a]pyridin-6-yl)methyl]cyclohexyl]-[(3S)-3-(6-methylpyridin-3-yl)-1,2-oxazolidin-2-yl]methanone,
(342) trans-[(3S)-3-(5-fluoro-6-methylpyridin-3-yl)-1,2-oxazolidin-2-yl]-[4-[(8-methoxy-2-methyl-[1,2,4]triazolo[1,5-a]pyridin-6-yl)methyl]cyclohexyl]methanone,
(343) trans-[(3S)-3-(5-fluoro-6-methylpyridin-3-yl)-1,2-oxazolidin-2-yl]-[4-[(2-methylpyrimidin-5-yl)methyl]cyclohexyl]methanone,
(344) trans-[(3S)-3-(6-methylpyridin-3-yl)-1,2-oxazolidin-2-yl]-[4-[(2-methylpyrimidin-5-yl)methyl]cyclohexyl]methanone,
(345) trans-[(3S)-3-(5-fluoropyridin-3-yl)-1,2-oxazolidin-2-yl]-[4-[(8-methoxy-2-methyl-[1,2,4]triazolo[1,5-a]pyridin-6-yl)methyl]cyclohexyl]methanone,
(346) trans-[4-[(2-methylpyrimidin-5-yl)methyl]cyclohexyl]-[(3S)-3-pyrazin-2-yl-1,2-oxazolidin-2-yl]methanone,
(347) trans-[(3S)-3-(6-methylpyridin-3-yl)-1,2-oxazolidin-2-yl]-[4-[(8-methyl-[1,2,4]triazolo[1,5-a]pyridin-6-yl)methyl]cyclohexyl]methanone,
(348) trans-[4-[[4-(3-hydroxyoxetan-3-yl)phenyl]methyl]cyclohexyl]-[(3S)-3-(6-methylpyridin-3-yl)-1,2-oxazolidin-2-yl]methanone,
(349) trans-[4-[(2-methylimidazo[1,2-b]pyridazin-7-yl)methyl]cyclohexyl]-[(3S)-3-pyrazin-2-yl-1,2-oxazolidin-2-yl]methanone,
(350) trans-[4-(8-Fluoro-2-methyl-imidazo[1,2-a]pyridin-6-ylmethyl)-cyclohexyl]-((S)-3-pyrazin-2-yl-isoxazolidin-2-yl)-methanone,
(351) trans-[4-[(8-fluoro-2-methylimidazo[1,2-a]pyridin-6-yl)methyl]cyclohexyl]-[(3S)-3-(6-methylpyridin-3-yl)-1,2-oxazolidin-2-yl]methanone,
(352) trans-[4-[[4-(3-hydroxyoxetan-3-yl)phenyl]methyl]cyclohexyl]-[(3S)-3-pyrazin-2-yl-1,2-oxazolidin-2-yl]methanone,
(353) trans-[4-[(2-methylimidazo[1,2-b]pyridazin-7-yl)methyl]cyclohexyl]-[(3S)-3-(6-methylpyridin-3-yl)-1,2-oxazolidin-2-yl]methanone,
(354) trans-[4-(2,5-Dimethyl-pyridin-4-ylmethyl)-cyclohexyl]-[(S)-3-(6-methyl-pyridin-3-yl)-isoxazolidin-2-yl]-methanone,
(355) trans-[4-[(2-methyl-[1,2,4]triazolo[1,5-a]pyridin-7-yl)methyl]cyclohexyl]-[(3S)-3-(5-fluoro-6-methyl-3-pyridyl)isoxazolidin-2-yl]-methanone
(356) trans-[4-(2,5-Dimethyl-pyridin-4-ylmethyl)-cyclohexyl]-((S)-3-pyrazin-2-yl-isoxazolidin-2-yl)-methanone,
(357) trans-[4-[(6-methylpyridazin-3-yl)methyl]cyclohexyl]-[(3S)-3-pyrazin-2-ylisoxazolidin-2-yl]methanone,
(358) trans-[4-[(3,5-dimethyl-[1,2,4]triazolo[4,3-a]pyridin-6-yl)methyl]cyclohexyl]-[(3S)-3-pyrazin-2-yl-1,2-oxazolidin-2-yl]methanone,
(359) trans-[4-[(8-fluoro-3-methyl-[1,2,4]triazolo[4,3-a]pyridin-6-yl)methyl]cyclohexyl]-[(3S)-3-pyrazin-2-yl-1,2-oxazolidin-2-yl]methanone,
(360) trans-(S)-3-(5-fluoro-6-methylpyridin-3-yl)-1,2-oxazolidin-2-yl]-[4-[(2-methyl-1,3-thiazol-4-yl)methyl]cyclohexyl]methanone,
(361) trans-[4-(2,8-Dimethyl-[1,2,4]triazolo[1,5-a]pyridin-6-ylmethyl)-cyclohexyl]-[(S)-3-(2-methyl-thiazol-4-yl)-isoxazolidin-2-yl]-methanone,
(362) trans-[(S)-3-(2-Methyl-thiazol-4-yl)-isoxazolidin-2-yl]-[4-(8-methyl-[1,2,4]triazolo[1,5-a]pyridin-6-ylmethyl)-cyclohexyl]-methanone,
(363) trans-[4-(8-Fluoro-3-methyl-[1,2,4]triazolo[4,3-a]pyridin-6-ylmethyl)-cyclohexyl]-[(S)-3-(2-methyl-thiazol-4-yl)-isoxazolidin-2-yl]-methanone,
(364) trans-[4-(8-Fluoro-3-methyl-[1,2,4]triazolo[4,3-a]pyridin-6-ylmethyl)-cyclohexyl]-[(S)-3-(6-methyl-pyridin-3-yl)-isoxazolidin-2-yl]-methanone,
(365) trans-[4-(3,8-Dimethyl-[1,2,4]triazolo[4,3-a]pyridin-6-ylmethyl)-cyclohexyl]-[(S)-3-(6-methyl-pyridin-3-yl)-isoxazolidin-2-yl]-methanone,
(366) trans-[4-(2,5-Dimethyl-pyridin-4-ylmethyl)-cyclohexyl]-[(S)-3-(2-methyl-thiazol-4-yl)-isoxazolidin-2-yl]-methanone,
(367) trans-[4-(8-Methoxy-2-methyl-[1,2,4]triazolo[1,5-a]pyridin-6-ylmethyl)-cyclohexyl]-[(S)-3-(2-methyl-thiazol-4-yl)-isoxazolidin-2-yl]-methanone,
(368) trans-[4-(8-Methoxy-2-methyl-[1,2,4]triazolo[1,5-a]pyridin-6-ylmethyl)-cyclohexyl]-[(S)-3-(6-methyl-pyridin-3-yl)-isoxazolidin-2-yl]-methanone,
(369) trans-{4-[4-(3-Hydroxy-oxetan-3-yl)-benzyl]-cyclohexyl}-[(S)-3-(2-methyl-thiazol-4-yl)-isoxazolidin-2-yl]-methanone,
(370) trans-[4-(8-Fluoro-2-methyl-imidazo[1,2-a]pyridin-6-ylmethyl)-cyclohexyl]-[(S)-3-(2-methyl-thiazol-4-yl)-isoxazolidin-2-yl]-methanone,
(371) trans-[4-(2-Methyl-imidazo[1,2-b]pyridazin-7-ylmethyl)-cyclohexyl]-[(S)-3-(2-methyl-thiazol-4-yl)-isoxazolidin-2-yl]-methanone,
(372) trans-[(S)-3-(2-Methyl-thiazol-4-yl)-isoxazolidin-2-yl]-[4-(2-methyl-[1,2,4]triazolo[1,5-a]pyridin-7-ylmethyl)-cyclohexyl]-methanone,
(373) trans-[(S)-3-(6-Methyl-pyridin-3-yl)-isoxazolidin-2-yl]-[4-(2-methyl-[1,2,4]triazolo[1,5-a]pyridin-7-ylmethyl)-cyclohexyl]-methanone,
(374) trans-[4-(2-Methyl-[1,2,4]triazolo[1,5-a]pyridin-7-ylmethyl)-cyclohexyl]-((S)-3-pyrazin-2-yl-isoxazolidin-2-yl)-methanone,
(375) trans-[4-(3,5-Dimethyl-[1,2,4]triazolo[4,3-a]pyridin-6-ylmethyl)-cyclohexyl]-[(S)-3-(2-methyl-thiazol-4-yl)-isoxazolidin-2-yl]-methanone,
(376) trans-[4-(3,5-Dimethyl-[1,2,4]triazolo[4,3-a]pyridin-6-ylmethyl)-cyclohexyl]-[(S)-3-(6-methyl-pyridin-3-yl)-isoxazolidin-2-yl]-methanone,
(377) trans-[4-[(2,5-dimethylpyridin-4-yl)methyl]cyclohexyl]-[(3S)-3-(2-methyl-1,3-oxazol-4-yl)-1,2-oxazolidin-2-yl]methanone,
(378) trans-[4-[(2,8-dimethyl-[1,2,4]triazolo[1,5-a]pyridin-6-yl)methyl]cyclohexyl]-[(3S)-3-(5-methylpyridin-3-yl)-1,2-oxazolidin-2-yl]methanone,
(379) trans-[4-[(8-fluoro-2-methyl-[1,2,4]triazolo[1,5-a]pyridin-6-yl)methyl]cyclohexyl]-[(3S)-3-(5-methylpyrazin-2-yl)-1,2-oxazolidin-2-yl]methanone,
(380) trans-[4-[(8-fluoro-2-methyl-[1,2,4]triazolo[1,5-a]pyridin-6-yl)methyl]cyclohexyl]-[(3S)-3-(5-fluoropyridin-3-yl)-1,2-oxazolidin-2-yl]methanone,
(381) trans-[4-[(8-chloro-2-methyl-[1,2,4]triazolo[1,5-a]pyridin-6-yl)methyl]cyclohexyl]-[(3S)-3-(5-fluoro-6-methylpyridin-3-yl)-1,2-oxazolidin-2-yl]methanone,
(382) trans-[4-[(8-chloro-2-methyl-[1,2,4]triazolo[1,5-a]pyridin-6-yl)methyl]cyclohexyl]-[(3S)-3-(5-fluoropyridin-3-yl)-1,2-oxazolidin-2-yl]methanone,
(383) trans-[4-[(8-methoxy-2-methyl-[1,2,4]triazolo[1,5-a]pyridin-6-yl)methyl]cyclohexyl]-[(3S)-3-(5-methylpyridin-3-yl)-1,2-oxazolidin-2-yl]methanone,
(384) trans-[4-[(5-fluoro-2-methylpyridin-4-yl)methyl]cyclohexyl]-[(3S)-3-(5-methylpyrazin-2-yl)-1,2-oxazolidin-2-yl]methanone,
(385) trans-[4-[(5-chloro-2-methylpyridin-4-yl)methyl]cyclohexyl]-[(3S)-3-(5-methylpyrazin-2-yl)-1,2-oxazolidin-2-yl]methanone,
(386) trans-[4-[(5-fluoro-2-methylpyridin-4-yl)methyl]cyclohexyl]-[(3S)-3-(6-methylpyridin-3-yl)-1,2-oxazolidin-2-yl]methanone,
(387) trans-[4-[(6-methylpyridazin-3-yl)methyl]cyclohexyl]-[(3S)-3-(6-methylpyridin-3-yl)-1,2-oxazolidin-2-yl]methanone,
(388) trans-[4-[(6-methylpyridazin-3-yl)methyl]cyclohexyl]-[(3S)-3-(5-fluoro-6-methylpyridin-3-yl)-1,2-oxazolidin-2-yl]methanone,
(389) trans-[4-[(5-chloro-2-methylpyridin-4-yl)methyl]cyclohexyl]-[(3S)-3-(6-methylpyridin-3-yl)-1,2-oxazolidin-2-yl]methanone,
(390) trans-[4-[(8-fluoro-[1,2,4]triazolo[1,5-a]pyridin-6-yl)methyl]cyclohexyl]-[(3S)-3-(5-methylpyridin-3-yl)-1,2-oxazolidin-2-yl]methanone,
(391) trans-[4-[(2-amino-[1,2,4]triazolo[1,5-a]pyridin-6-yl)methyl]cyclohexyl]-[(3S)-3-(6-methylpyridin-3-yl)-1,2-oxazolidin-2-yl]methanone,
(392) trans-[(3S)-3-(5-fluoro-6-methylpyridin-3-yl)-1,2-oxazolidin-2-yl]-[4-[(8-fluoro-[1,2,4]triazolo[1,5-a]pyridine-6-yl)methyl]cyclohexyl]methanone,
(393) trans-[4-[(2,4-dimethylpyrimidin-5-yl)methyl]cyclohexyl]-[(3S)-3-(5-fluoro-6-methylpyridin-3-yl)-1,2-oxazolidin-2-yl]methanone,
(394) trans-[4-[(5-fluoro-2-methylpyridin-4-yl)methyl]cyclohexyl]-[(3S)-3-pyrazin-2-yl-1,2-oxazolidin-2-yl]methanone,
(395) trans-[(3S)-3-(5-methylpyrazin-2-yl)-1,2-oxazolidin-2-yl]-[4-[(2-methylpyrazol-3-yl)methyl]cyclohexyl]methanone,
(396) trans-[4-[(5-chloro-2-methylpyridin-4-yl)methyl]cyclohexyl]-[(3S)-3-pyrazin-2-yl-1,2-oxazolidin-2-yl]methanone,
(397) trans-[4-[(2-methylpyrazol-3-yl)methyl]cyclohexyl]-[(3S)-3-(6-methylpyridin-3-yl)-1,2-oxazolidin-2-yl]methanone,
(398) trans-[(3S)-3-(5-fluoro-6-methylpyridin-3-yl)-1,2-oxazolidin-2-yl]-[4-[(6-methylpyridazin-4-yl)methyl]cyclohexyl]methanone,
(399) trans-[(3S)-3-(5-fluoro-6-methylpyridin-3-yl)-1,2-oxazolidin-2-yl]-[4-[[2-methyl-4-(trifluoromethyl)pyrimidin-5-yl]methyl]cyclohexyl]methanone,
(400) trans-[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone,
(401) trans-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone,
(402) cis-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone,
(403) cis-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone,
(404) trans-[(3S)-3-(5-fluoro-6-methylpyridin-3-yl)-1,2-oxazolidin-2-yl]-[4-[(2-methylpyrazol-3-yl)methyl]cyclohexyl]methanone,
(405) cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone,
(406) cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone,
(407) cis-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone,
(408) trans-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone,
(409) trans-[(3S)-3-(5-methylpyrazin-2-yl)-1,2-oxazolidin-2-yl]-[4-[(8-methyl-[1,2,4]triazolo[1,5-a]pyridin-6-yl)methyl]cyclohexyl]methanone,
(410) trans-[4-[(1,3-dimethylpyrazolo[4,3-b]pyridin-6-yl)methyl]cyclohexyl]-[(3S)-3-(5-methylpyrazin-2-yl)-1,2-oxazolidin-2-yl]methanone, and
(411) trans-[4-[(2,8-dimethyl-[1,2,4]triazolo[1,5-a]pyridin-6-yl)methyl]cyclohexyl]-[(3S)-3-(6-methylpyrazin-2-yl)isoxazolidin-2-yl]methanone;
   or a pharmaceutically acceptable salt, solvate, or stereoisomer thereof.

Among the compounds of formula (I) mention may be made in particular of the following compounds: compound (1), (2), (3), (7), (8), (10), (12), (13), (14), (15), (16), (17), (18), (19), (20), (21), (22), (23), (26), (27), (28), (29), (31), (32), (33), (35), (36), (37), (38), (39), (43), (44), (45), (46), (47), (48), (49), (50), (51), (52), (53), (54), (55), (57), (58), (60), (61), (62), (63), (64), (66), (67), (68), (69), (72), (73), (75), (76), (77), (79), (83), (84), (85), (86), (87), (89), (93), (94), (95), (96), (99), (100), (101), (102), (103), (104), (105), (108), (109), (110), (111), (112), (113), (118), (120), (121), (123), (125), (126), (127), (128), (129), (130), (131), (132), (133), (134), (136), (137), (138), (139), (141), (142), (144), (145), (146), (147), (148), (149), (150), (152), (153), (154), (155), (156), (158), (159), (160), (161), (162), (163), (164), (165), (166), (169), (170), (171), (172), (173), (174), (175), (176), (177), (178), (179), (180), (181), (182), (183), (184), (185), (186), (190), (191), (192), (193), (194), (195), (197), (198), (200), (202), (204), (206), (208), (210), (211), (212), (213), (214), (215), (216), (217), (218), (219), (220), (221), (222), (223), (224), (225), (226), (227), (231), (236), (239), (242), (243), (244), (245), (249), (250), (251), (256), (257), (259), (260), (261), (264), (265), (266), (267), (268), (269), (273), (274), (275), (276), (277), (278), (279), (280), (281), (282), (283), (284), (285), (286), (287), (288), (290), (291), (292), (293), (294), (295), (296), (297), (298), (299), (300), (301), (302), (303), (304), (305), (306), (307), (308), (309), (310), (311), (312), (313), (314), (315), (316), (317), (318), (319), (320), (321), (322), (323), (324), (325), (326), (327), (328), (329), (330), (331), (332), (333), (334), (335), (336), (337), (338), (339), (340), (341), (342), (343), (344), (345), (346), (347), (348), (349), (350), (351), (352), (353), (354), (355), (356), (357), (358), (359), (360), (361), (362), (363), (364), (365), (366), (367), (368), (369), (370), (371), (372), (373), (374), (375), (376), (377), (378), (379), (380), (381), (382), (383), (384), (385), (386), (387), (388), (389), (390), (391), (392), (393), (394), (395), (396), (397), (398), (399), (400), (401), (402), (403), (404), (405) and (406), (407), (408), (409), (410) and (411),

or a pharmaceutically acceptable salt, solvate, or stereoisomer thereof.

According to another particular embodiment of the present disclosure, the compound of formula (I) is chosen from the group consisting of compounds (1), (3), (7), (17), (18), (20), (21), (22), (23), (26), (27), (28), (29), (31), (32), (33), (36), (37), (43), (44), (45), (46), (47), (48), (49), (52), (53), (54), (55), (60), (61), (62), (66), (67), (68), (69), (73), (76), (77), (79), (84), (85), (86), (87), (93), (94), (95), (96), (100), (101), (103), (104), (105), (109), (110), (112), (113), (125), (128), (130), (131), (132), (133), (138), (139), (141), (144), (148), (149), (150), (152), (153), (155), (156), (160), (161), (162), (163), (164), (165), (170), (171), (172), (173), (174), (175), (176), (177), (180), (190), (191), (192), (193), (194), (195), (197), (198), (204), (206), (208), (210), (211), (212), (213), (214), (215), (216), (217), (218), (219), (221), (223), (225), (227), (236), (242), (243), (244), (245), (249), (250), (251), (256), (257), (259), (260), (261), (264), (265), (266), (267), (268), (273), (275), (276), (277), (278), (279), (280), (281), (282), (283), (285), (286), (287), (288), (290), (291), (292), (293), (294), (295), (296), (297), (298), (299), (300), (303), (306), (307), (309), (310), (311), (312), (313), (314), (315), (316), (317), (318), (319), (320), (321), (322), (323), (324), (325), (326), (327), (328), (329), (330), (331), (332), (333), (334), (335), (336), (337), (338), (339), (340), (341), (342), (343), (344), (345), (346), (347), (348), (349), (350), (351), (352), (353), (354), (355), (356), (357), (358), (359), (360), (361), (362), (363), (364), (365), (366), (367), (368), (369), (370), (371), (372), (373), (374), (375), (376), (377), (378), (379), (380), (381), (382), (383), (384), (385), (386), (387), (388), (389), (390), (391), (392), (393), (394), (395), (396), (397), (398), (399), (400), (401), (402), (403), (404), (405) (406), (407), (408), (409), (410) and (411).
or a pharmaceutically acceptable salt, solvate, or stereoisomer thereof.

According to another particular embodiment of the present disclosure, the compound of formula (I) is chosen from the group consisting of compounds (1), (180), (260), (288), (298), (306), (307), (309), (310), (311), (312), (313), (314), (315), (316), (317), (318), (319), (320), (321), (322), (323) and (324) or a pharmaceutically acceptable salt, solvate, or stereoisomer thereof.

According to another particular embodiment of the present disclosure, the compound of formula (I) is chosen from the group consisting of compounds (316), (326), (327), (328), (332), (334), (335), (337), (339), (343), (354), (384), (385), (392), (393), (395) and (411) or a pharmaceutically acceptable salt, solvate, or stereoisomer thereof.

Some compounds of the disclosure are described with their structure in the below Table 1, which is merely illustrative and does not limit the scope of the present disclosure.

NMR and LC/MS data in table 1 were obtained according to methods which are detailed in the experimental part provided for the detailed examples synthesis.

TABLE 1
Structure, and analytical characterization of compounds (1) to (411)

| Cpd | | |
|---|---|---|
| No | Structure | Description |
| 1 | 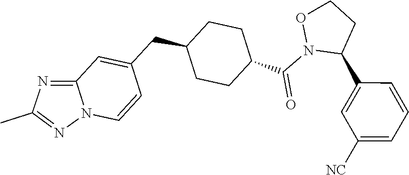 [SMILES: Cc1nc2cc(C[C@H]3CC[C@H](C(=O)N4OCC[C@H]4c4cccc(C#N)c4)CC3)ccn2n1] | ¹H NMR (400 MHz, DMSO-d₆): δ ppm 8.69 (d, J = 6.97 Hz, 1 H), 7.73 (dt, J = 7.24, 1.51, 1.51 Hz, 1 H), 7.69 (s, 1 H), 7.60 (d, J = 7.41 Hz, 1 H), 7.57 (d, J = 7.52 Hz, 1 H), 7.44 (s, 1 H), 6.95 (dd, J = 6.97, 1.71 Hz, 1 H), 5.34 (m, 1 H), 4.25 (td, J = 7.76, 7.76, 2.93 Hz, 1 H), 3.86 (m, 1 H), 2.88 (m, 1 H), 2.67 (m, 1 H), 2.60 (d, J = 6.85 Hz, 2 H) 2.43 (s, 3 H), 2.19 (m, 1 H), 1.87 (br d, J = 12.72 Hz, 1 H), 1.66 (m, 4 H), 1.31 (m, 2 H), 1.06 (m, 2 H). LC/MS: m/z [M + H]⁺ 430.3, Rt (I = 220 nm): 1.91 min (LC/MS method A), calc. m/z: 430.20. |
| 2 | 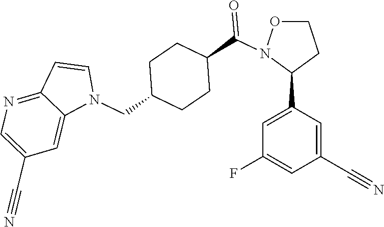 [SMILES: N#Cc1cc(F)cc([C@@H]2CCON2C(=O)[C@H]2CC[C@H](Cn3ccc4ncc(C#N)cc43)CC2)c1] | 1H NMR (600.05 MHz, DMSO-d6) d ppm 8.66 (s, 1 H), 8.65 (s, 1 H), 8.00 (d, J = 3.12 Hz, 1 H), 7.75 (br d, J = 8.44 Hz, 1 H), 7.58 (s, 1 H), 7.45 (br d, J = 9.72 Hz, 1 H), 6.74 (d, J = 2.57 Hz, 1 H), 5.35 (br t, J = 7.34, 7.34 Hz, 1 H), 4.24 (td, J = 7.84, 7.84, 3.03 Hz, 1 H), 4.14 (d, J = 7.34 Hz, 2 H), 3.86 (m, 1 H), 2.87 (dddd, J = 12.20, 9.17, 6.51, 2.93 Hz, 1 H), 2.67 (br s, 1 H), 2.61 (m, 1 H), 2.21 (m, 1 H), 1.86 (m, 2 H), 1.70 (br d, J = 11.0 Hz, 1 H), 1.53 (m, 2 H), 1.27 (m, 2 H), 1.11 (m, 2 H). LC/MS: m/z [M + H]⁺ 458.34, Rt (I = 220 nm): 2.08 min (LC/MS method A), calc. m/z: 458.51. |
| 3 | 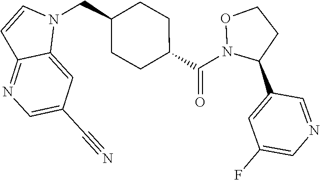 [SMILES: N#Cc1cnc2ccn(C[C@H]3CC[C@H](C(=O)N4OCC[C@H]4c4cncc(F)c4)CC3)c2c1] | 1H NMR (600.05 MHz, DMSO-d6) d ppm 8.67 (s, 1 H), 8.66 (s, 1 H), 8.48 (d, J = 2.75 Hz, 1 H), 8.38 (s, 1 H), 8.01 (d, J = 3.12 Hz, 1 H), 7.57 (d, J = 9.68 Hz, 1 H), 6.75 (d, J = 3.12 Hz, 1 H), 5.38 (br t, J = 7.43, 7.43 Hz, 1 H), 4.27 (td, J = 7.75, 7.75, 2.84 Hz, 2 H), 4.14 (d, J = 7.34 Hz, 2 H), 3.87 (m, 14 H), 2.88 (dddd, J = 12.17, 9.19, 6.51, 3.12 Hz, 1 H), 2.66 (br s, 1 H), 2.25 (m, 1 H), 1.85 (m, 2 H), 1.70 (br d, J = 11.00 Hz, 1 H), 1.52 (m, 2 H), 1.27 (m, 2 H), 1.11 (m, 2 H). LC/MS: m/z [M + H]⁺ 434.35, Rt (I = 220 nm): 1.8 min (LC/MS method A), calc. m/z: 434.49. |
| 4 | 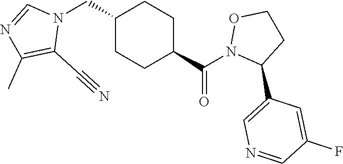 [SMILES: Cc1ncn(C[C@H]2CC[C@H](C(=O)N3OCC[C@H]3c3cncc(F)c3)CC2)c1C#N] | ¹H NMR (600 MHz, DMSO-d₆): δ ppm 8.49 (d, J = 2.75 Hz, 1 H), 8.39 (s, 1 H), 8.00 (s, 1 H), 7.58 (dt, J = 9.81, 2.15 Hz, 1 H), 5.39 (br t, J = 7.43 Hz, 1 H), 4.28 (m, 1 H), 3.95 (br d, J = 6.42 Hz, 2 H), 3.91 (m, 1 H), 2.89 (m, 1 H), 2.68 (br s, 1 H), 2.27 (s, 3 H), 2.25 (m, 1 H), 1.91 (br d, J = 12.84 Hz, 1 H), 1.74 (m, 2 H), 1.60 (m, 2 H), 1.30 (m, 2 H), 1.07 (m, 2 H). LC/MS: m/z [M + H]⁺ 398.4, Rt (I = 220 nm): 1.64 min (LC/MS method A), calc. m/z: 398.45. |
| 5 | 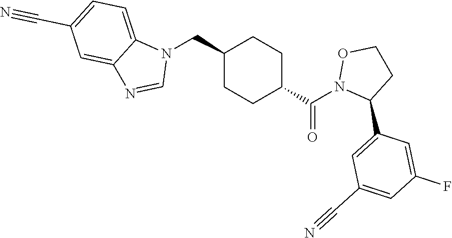 [SMILES: N#Cc1cc(F)cc([C@@H]2CCON2C(=O)[C@H]2CC[C@H](Cn3cnc4cc(C#N)ccc43)CC2)c1] | 1H NMR (600.05 MHz, DMSO-d6) d ppm 8.58 (s, 1 H), 8.24 (s, 1 H), 7.91 (d, J = 8.44 Hz, 1 H), 7.75 (br d, J = 8.44 Hz, 1 H), 7.70 (dd, J = 8.44, 1.28 Hz, 1 H), 7.58 (s, 1 H), 7.45 (br d, J = 9.72 Hz, 1 H), 5.35 (br t, J = 7.43, 7.43 Hz, 1 H), 4.24 (td, J = 7.70, 7.70, 2.93 Hz, 1 H), 4.21 (d, J = 7.15 Hz, 2 H), 3.85 (m, 2 H), 2.87 (m, 1 H), 2.67 (br s, 1 H), 2.21 (m, 1 H), 1.86 (m, 2 H), 1.70 (br d, J = 10.64 Hz, 1 H), 1.59 (m, 2 H), 1.28 (m, 2 H), 1.12 (m, 2 H). LC/MS: m/z [M + H]⁺ 458.4, Rt (I = 220 nm): 2 min (LC/MS method A), calc. m/z: 458.51. |
| 6 | 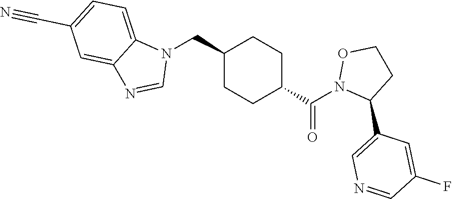 [SMILES: N#Cc1ccc2c(c1)ncn2C[C@H]1CC[C@H](C(=O)N2OCC[C@H]2c2cncc(F)c2)CC1] | ¹H (600 MHz, DMSO-d6) δ = 8.57 (s, 1H), 8.48 (d, J = 2.6 Hz, 1H), 8.37 (s, 1H), 8.23 (s, 1H), 7.91 (d, J = 8.4 Hz, 1H), 7.70 (dd, J = 8.4, 1.3 Hz, 1H), 7.56 (br d, J = 9.9 Hz, 1H), 5.41-5.32 (m, 1H), 4.26 (td, J = 7.7, 2.9 Hz, 1H), 4.20 (d, J = 7.2 Hz, 2H), 3.90-3.84 (m, 1H, below water signal), 2.92-2.84 (m, 1H), 2.71-2.62 (m, 1H), 2.28-2.21 (m, 1H), 1.90-1.81 (m, 2H), 1.74- 1.67 (m, 1H), 1.63-1.52 (m, 2H), 1.33-1.23 (m, 2H), 1.18-1.04 (m, 2H) LC/MS: m/z [M + H]⁺ 434.4, Rt (I = 220 nm): 1.71 min (LC/MS method A), calc. m/z: 434.49. |
| 7 | 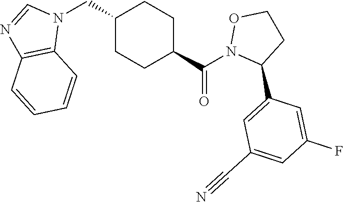 [SMILES: N#Cc1cc(F)cc([C@@H]2CCON2C(=O)[C@H]2CC[C@H](Cn3cnc4ccccc43)CC2)c1] | ¹H (400 MHz, DMSO-d6) δ = 9.36 (s, 1H), 8.04-7.93 (m, 1H), 7.91-7.81 (m, 1H), 7.78-7.68 (m, 1H), 7.60- 7.50 (m, 3H), 7.48-7.42 (m, 1H), 5.43-5.30 (m, 1H), 4.33 (d, J = 7.1 Hz, 2H), 4.27-4.18 (m, 1H), 3.89- 3.81 (m, 1H, below water signal), 2.96-2.81 (m, 1H), 2.76-2.63 (m, 1H), 2.28-2.14 (m, 1H), 1.99- 1.83 (m, 2H), 1.77-1.55 (m, 3H), 1.38-1.20 (m, 2H), 1.22-1.08 (m, 2H) LC/MS: m/z [M + H]⁺ 433.2, Rt (I = 220 nm): 1.53 min (LC/MS method A), calc. m/z: 433.50. |
| 8 | 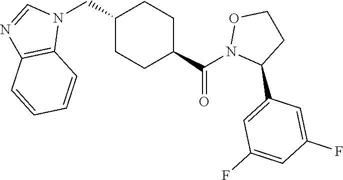 [SMILES: O=C([C@H]1CC[C@H](Cn2cnc3ccccc32)CC1)N1OCC[C@H]1c1cc(F)cc(F)c1] | 1H NMR (DMSO-d6) δ: 9.31 (s, 1H), 7.99 (d, J = 7.1 Hz, 1H), 7.83 (d, J = 7.2 Hz, 1H), 7.51-7.59 (m, 2H), 7.12 (tt, J = 9.3, 2.3 Hz, 1H), 6.93-6.99 (m, 2H), 5.27- 5.34 (m, 1H), 4.32 (d, J = 7.1 Hz, 2H), 4.23 (td, J = 7.7, 3.0 Hz, 1H), 3.78-3.91 (m, 2H), 2.81-2.94 (m, 2H), 2.60-2.74 (m, 1H), 2.28-2.38 (m, 1H), 2.08-2.27 (m, 1H), 1.91 (br d, J = 12.0 Hz, 2H), 1.59-1.77 (m, 3H), 1.08-1.36 (m, 4H). LC/MS: m/z [M + H]⁺ 426.3, Rt (I = 220 nm): 1.64 min (LC/MS method A), calc. m/z: 426.48. |
| 9 | 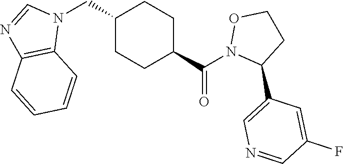 [SMILES: O=C([C@H]1CC[C@H](Cn2cnc3ccccc32)CC1)N1OCC[C@H]1c1cncc(F)c1] | 1H NMR (400.23 MHz, DMSO-d6) d ppm 9.41 (s, 1 H), 8.47 (m, 1 H), 8.02 (d, J = 7.08 Hz, 1 H), 7.85 (d, J = 6.94 Hz, 1 H), 7.58 (m, 1 H), 5.38 (m, 1 H), 4.34 (d, J = 6.97 Hz, 1 H), 4.28 (td, J = 7.76, 7.76, 3.18 Hz, 1 H), 4.01 (m, 1 H), 3.88 (m, 2 H), 2.89 (dddd, J = 12.09, 9.15, 6.51, 3.06 Hz, 1 H), 2.67 (m, 1 H), 2.27 (m, 1 H), 1.91 (m, 1 H), 1.73 (br d, J = 12.47 Hz, 1 H), 1.66 (br d, J = 11.98 Hz, 1 H), 1.29 (m, 1 H), 1.16 (dq, J = 13.14, 12.98, 12.98, 12.98 Hz, 1 H). LC/MS: m/z [M + H]⁺ 409.2, Rt (I = 220 nm): 1.28 min (LC/MS method A), calc. m/z: 409.48. |
| 10 | 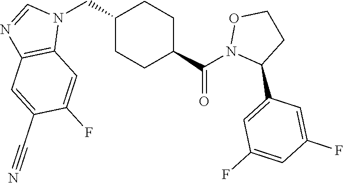 [SMILES: N#Cc1cc2ncn(C[C@H]3CC[C@H](C(=O)N4OCC[C@H]4c4cc(F)cc(F)c4)CC3)c2cc1F] | 1H NMR (400.23 MHz, DMSO-d6) d ppm 8.49 (s, 1 H), 8.29 (d, J = 5.75 Hz, 1 H), 7.95 (d, J = 9.90 Hz, 1 H), 7.11 (tt, J = 9.31, 2.31, 2.31 Hz, 1 H), 6.96 (m, 2 H), 5.31 (m, 1 H), 4.18 (m, 3 H), 3.84 (m, 1 H), 2.85 (m, 1 H), 2.67 (br d, J = 1.83 Hz, 1 H), 2.17 (m, 1 H), 1.86 (m, 2 H), 1.69 (br d, J = 11.86 Hz, 1 H), 1.57 (m, 2 H), 1.29 (m, 2 H), 1.11 (m, 2 H). LC/MS: m/z [M + H]⁺ 469.2, Rt (I = nm): 2.26 min (LC/MS method A), calc. m/z: 469.48. |
| 11 | 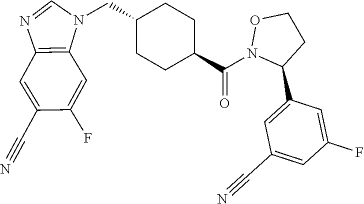 [SMILES: N#Cc1cc(F)cc([C@@H]2CCON2C(=O)[C@H]2CC[C@H](Cn3cnc4cc(C#N)c(F)cc43)CC2)c1] | ¹H NMR (400 MHz, DMSO-d₆): δ ppm 8.49 (s, 1 H), 8.29 (d, J = 5.75 Hz, 1 H), 7.96 (d, J = 9.0 Hz, 1 H), 7.75 (br d, J = 7.95 Hz, 1 H), 7.58 (s, 1 H), 7.45 (br d, J = 9.54 Hz, 1 H), 5.35 (dd, J = 8.25, 6.66 Hz, 1 H), 4.24 (m, 1 H), 4.15 (d, J = 7.09 Hz, 2 H), 3.86 (m, 1 H), 2.87 (m, 1 H), 2.66 (m, 1 H), 2.21 (m, 1 H), 1.86 (m, 2 H), 1.71 (br d, J = 11.74 Hz, 1 H), 1.58 (br t, J = 12.65, 2 H), 1.29 (m, 2 H), 1.11 (m, 2 H). LC/MS: m/z [M + H]⁺ 476.3, Rt (I = 220 nm): 2.13 min (LC/MS method A), calc. m/z: 476.50. |
| 12 | 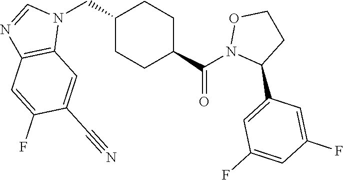 [SMILES: N#Cc1cc2c(cc1F)ncn2C[C@H]1CC[C@H](C(=O)N2OCC[C@H]2c2cc(F)cc(F)c2)CC1] | ¹H NMR (400 MHz, DMSO-d₆): δ ppm 8.57 (s, 1 H), 8.43 (d, J = 5.62 Hz, 1 H), 7.79 (d, J = 10.27 Hz, 1 H), 7.12 (tt, J = 9.32, 9.32, 2.29, 2.29 Hz, 1 H), 6.96 (m, 2 H), 5.31 (dd, J = 8.56, 6.36 Hz, 1 H), 4.23 (td, J = 7.76, 7.76, 3.06 Hz, 1 H), 4.17 (d, J = 7.21 Hz, 2 H), 3.84 (m, 1 H), 2.85 (m, 1 H), 2.67 (m, 1 H), 2.17 (m, 1 H), 1.89 (m, 2 H), 1.70 (br d, J = 11.13 Hz, 1 H), 1.57 (br t, J = 11.80, 11.80 Hz, 2 H), 1.29 (m, 2 H), 1.12 (m, 2 H). LC/MS: m/z [M + H]⁺ 469.2, Rt (I = 220 nm): 2.25 min (LC/MS method A), calc. m/z: 469.48. |
| 13 | 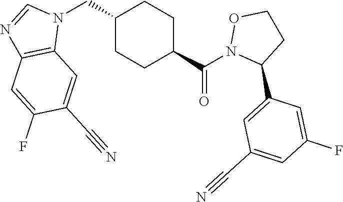 [SMILES: N#Cc1cc(F)cc([C@@H]2CCON2C(=O)[C@H]2CC[C@H](Cn3cnc4cc(F)c(C#N)cc43)CC2)c1] | ¹H NMR (400 MHz, DMSO-d₆): δ ppm 8.57 (s, 1 H), 8.43 (d, J = 5.75 Hz, 1 H), 7.78 (m, 2 H), 7.58 (s, 1 H), 7.46 (br d, J = 9.54 Hz, 1 H), 5.35 (m, 1 H), 4.25 (td, J = 7.73, 7.73, 2.87 Hz, 1 H), 4.17 (d, J = 7.21 Hz, 2 H), 3.86 (m, 3 H), 3.67 (br s, 35 H), 2.87 (m, 1 H), 2.67 (br d, J = 1.71 Hz, 1 H), 2.21 (m, 1 H), 1.89 (br d, J = 11.37 Hz, 2 H), 1.71 (br d, J = 11.86 Hz, 1 H), 1.57 (br t, J = 12.04, 12.04 Hz, 2 H), 1.30 (m, 2 H), 1.11 (m, 2 H). LC/MS: m/z [M + H]⁺ 476.2, Rt (I = 220 nm): 2.12 min (LC/MS method A), calc. m/z: 476.50. |
| 14 | 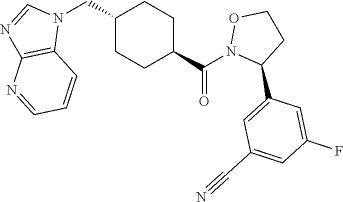 [SMILES: N#Cc1cc(F)cc([C@@H]2CCON2C(=O)[C@H]2CC[C@H](Cn3cnc4ncccc43)CC2)c1] | 1H NMR (400.23 MHz, DMSO-d6) d ppm 8.78 (d, J = 8.31 Hz, 1 H), 8.67 (d, J = 5.62 Hz, 1 H), 8.21 (d, J = 3.18 Hz, 1 H), 7.77 (dd, J = 2.26, 1.28 Hz, 1 H), 7.75 (s, 1 H), 7.66 (dd, J = 8.31, 5.62 Hz, 1 H), 7.57 (s, 1 H), 7.45 (br d, J = 9.41 Hz, 1 H), 6.86 (d, J = 3.06 Hz, 1 H), 5.34 (dd, J = 8.62, 6.42 Hz, 1 H), 4.26 (m, 3 H), 3.86 (m, 1 H), 2.87 (dddd, J = 12.17, 9.17, 6.48, 3.00 Hz, 1 H), 2.67 (m, 1 H), 2.64 (br s, 1 H), 2.21 (m, 1 H), 1.88 (m, 2 H), 1.70 (br d, J = 12.23 Hz, 1 H), 1.53 (br s, 2 H), 1.27 (m, 2 H), 1.08 (m, 2 H). LC/MS: m/z [M + H]⁺ 433.3, Rt (I = 220 nm): 1.38 min (LC/MS method A), calc. m/z: 433.50. |
| 15 | 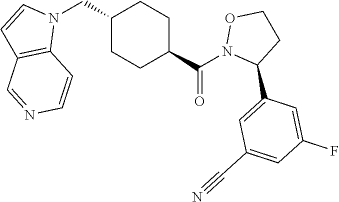 [SMILES: N#Cc1cc(F)cc([C@@H]2CCON2C(=O)[C@H]2CC[C@H](Cn3ccc4cnccc43)CC2)c1] | 1H NMR (400.23 MHz, DMSO-d6) d ppm 14.72 (br s, 1 H), 9.27 (s, 1 H), 8.48 (d, J = 6.85 Hz, 1 H), 8.23 (d, J = 6.85 Hz, 1 H), 7.98 (d, J = 3.42 Hz, 1 H), 7.76 (d, J = 8.64 Hz, 1 H), 7.57 (s, 1 H), 7.45 (d, J = 10.36 Hz, 1 H), 7.09 (d, J = 3.06 Hz, 1 H), 5.34 (dd, J = 8.50, 6.54 Hz, 1 H), 4.27 (m, 3 H), 3.86 (m, 1 H), 3.48 (u), 2.87 (dddd, J = 12.12, 9.12, 6.45, 3.00 Hz, 1 H), 2.66 (m, 1 H), 2.21 (m, 1 H), 1.89 (m, 2 H), 1.71 (br d, J = 11.98 Hz, 1 H) 1.54 (br s, 2 H), 1.27 (m, 2 H), 1.16 (m, 2 H). LC/MS: m/z [M + H]⁺ 433.3, Rt (I = 220 nm): 1.38 min (LC/MS method A), calc. m/z: 433.50. |
| 16 | 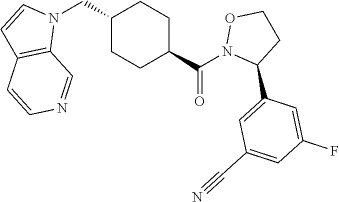 [SMILES: N#Cc1cc(F)cc([C@@H]2CCON2C(=O)[C@H]2CC[C@H](Cn3ccc4ccncc43)CC2)c1] | ¹H NMR (400 MHz, DMSO-d₆): δ ppm 14.95 (br s, 1 H), 9.45 (s, 1 H), 8.31 (m, 2 H), 8.14 (d, J = 6.36 Hz, 1 H), 7.76 (d, J = 8.66 Hz, 1 H), 7.58 (s, 1 H), 7.45 (br d, J = 9.54 Hz, 1 H), 6.98 (d, J = 2.81 Hz, 1 H), 5.35 (dd, J = 8.38, 6.66 Hz, 1 H), 4.33 (d, J = 7.34 Hz, 2 H), 4.25 (td, J = 7.73, 7.73, 2.87 Hz, 1 H), 3.86 (m, 1 H), 2.87 (m, 1 H), 2.68 (m, 1 H), 2.21 (m, 1 H), 1.89 (br d, J = 11.13 Hz, 2 H), 1.71 (br d, J = 11.74 Hz, 1 H), 1.54 (m, 2 H), 1.28 (m, 2 H), 1.16 (m, 2 H). LC/MS: m/z [M + H]⁺ 433.3, Rt (I = 220 nm): 1.4 min (LC/MS method A), calc. m/z: 433.50. |
| 17 | 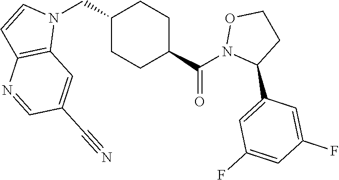 [SMILES: N#Cc1cnc2ccn(C[C@H]3CC[C@H](C(=O)N4OCC[C@H]4c4cc(F)cc(F)c4)CC3)c2c1] | ¹H NMR (400 MHz, DMSO-d₆): δ ppm 8.67 (s, 1 H), 8.00 (d, J = 3.18 Hz, 1 H), 7.11 (tt, J = 9.34, 9.34, 2.28, 2.28 Hz, 1 H), 6.95 (br d, J = 6.48 Hz, 2 H), 6.74 (d, J = 2.93 Hz, 1 H), 5.31 (dd, J = 8.56, 6.36 Hz, 1 H), 4.23 (td, J = 7.70, 7.70, 2.93 Hz, 1 H), 4.14 (d, J = 7.21 Hz, 2 H), 3.84 (m, 1 H), 2.85 (m, 1 H), 2.67 (br d, J = 1.83 Hz, 1 H), 2.17 (m, 1 H), 1.86 (m, 2 H), 1.69 (br d, J = 12.35 Hz, 1 H), 1.53 (br t, J = 13.51, 13.51 Hz, 2 H), 1.27 (br t, J = 12.0, 12.10 Hz, 2 H), 1.10 (m, 2 H). LC/MS: m/z [M + H]⁺ 451.3, Rt (I = 220 nm): 2.25 min (LC/MS method A). calc. m/z: 451.49. |
| 18 | 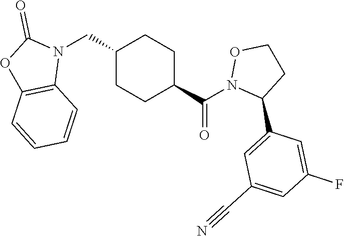 [SMILES: N#Cc1cc(F)cc([C@@H]2CCON2C(=O)[C@H]2CC[C@H](Cn3c(=O)oc4ccccc43)CC2)c1] | 1H NMR (400.23 MHz, DMSO-d6) d ppm 7.75 (d, J = 8.64 Hz, 1 H), 7.59 (s, 1 H), 7.46 (br d, J = 9.66 Hz, 1 H), 7.34 (d, J = 4.40 Hz, 1 H), 7.32 (m, 1 H), 7.18 (m, 2 H), 5.36 (dd, J = 8.44, 6.48 Hz, 1 H), 4.25 (td, J = 7.73, 7.73, 3.00 Hz, 1 H), 3.86 (m, 1 H), 3.68 (d, J = 7.09 Hz, 2 H), 2.87 (dddd, J = 12.13, 9.14, 6.48, 3.06 Hz, 1 H), 2.69 (m, 1 H), 2.21 (m, 1 H), 1.91 (br d, J = 12.35 Hz, 1 H), 1.81 (td, J = 7.27, 7.27, 3.42 Hz, 1 H), 1.72 (br d, J = 8.68 Hz, 3 H), 1.30 (m, 2 H), 1.15 (m, 2 H). LC/MS: m/z [M + H]⁺ 450.2, Rt (I = 220 nm): 2.32 min (LC/MS method A), calc. m/z: 450.48. |
| 19 | 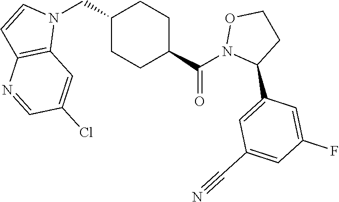 [SMILES: N#Cc1cc(F)cc([C@@H]2CCON2C(=O)[C@H]2CC[C@H](Cn3ccc4ncc(Cl)cc43)CC2)c1] | ¹H NMR (400 MHz, DMSO-d₆): δ ppm 8.39 (d, J = 1.96 Hz, 1 H), 8.31 (s, 1 H), 7.78 (d, J = 3.30 Hz, 1 H), 7.75 (br m, 1 H), 7.58 (s, 1 H), 7.45 (br d, J = 9.54 Hz, 1 H), 6.63 (d, J = 3.18 Hz, 1 H), 5.35 (m, 1 H), 4.24 (td, J = 7.76, 7.76, 2.93 Hz, 1 H), 4.09 (d, J = 7.21 Hz, 2 H), 3.85 (m, 1 H), 2.87 (m, 1 H), 2.66 (m, 1 H), 2.21 (m, 1 H), 1.83 (m, 2 H), 1.70 (br d, J = 12.84 Hz, 1 H), 1.54 (m, 2 H), 1.27 (m, 2 H), 1.11 (m, 2 H). LC/MS: m/z [M + H]⁺ 467.2, Rt (I = 220 nm): 2.2 min (LC/MS method A), calc. m/z: 467.934. |
| 20 | 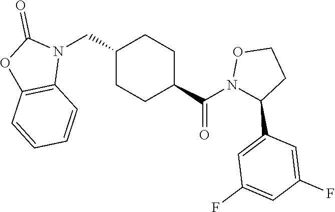 [SMILES: O=c1oc2ccccc2n1C[C@H]1CC[C@H](C(=O)N2OCC[C@H]2c2cc(F)cc(F)c2)CC1] | ¹H NMR (400 MHz, DMSO-d₆): δ ppm 7.34 (d, J = 4.65 Hz, 2 H), 7.32 (m, 1 H), 7.16 (m, 2 H), 6.96 (m, 2 H), 5.32 (dd, J = 8.44, 6.36 Hz, 1 H), 4.23 (td, J = 7.70, 7.70, 3.06 Hz, 1 H), 3.84 (m, 1 H), 3.67 (d, J = 7.09 Hz, 2 H), 2.86 (m, 1 H), 2.68 (m, 1 H), 2.67 (m, 1 H), 2.17 (m, 1 H), 1.90 (br d, J = 11.62 Hz, 1 H), 1.76 (m, 4 H), 1.30 (m, 2 H), 1.15 (m, 2 H). LC/MS: m/z [M + H]⁺ 443.2, Rt (I = 220 nm): 2.45 min (LC/MS method A), calc. m/z: 443.46. |
| 21 | 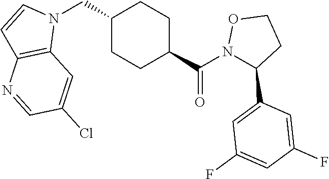 [SMILES: O=C([C@H]1CC[C@H](Cn2ccc3ncc(Cl)cc32)CC1)N1OCC[C@H]1c1cc(F)cc(F)c1] | ¹H NMR (400 MHz, DMSO-d₆): δ ppm 8.39 (d, J = 1.96 Hz, 1 H), 8.31 (s, 1 H), 7.78 (d, J = 3.30 Hz, 1 H), 7.11 (tt, J = 9.35, 9.35, 2.32, 2.32 Hz, 1 H), 6.96 (m, 2 H), 6.63 (d, J = 2.81 Hz, 1 H), 5.31 (dd, J = 8.56, 6.48 Hz, 1 H), 4.23 (td, J = 7.76, 7.76, 2.93 Hz, 1 H), 4.09 (d, J = 7.21 Hz, 2 H), 2.85 (m, 1 H), 2.67 (br d, J = 1.83 Hz, 1 H), 2.17 (m, 1 H), 1.83 (m, 2 H), 1.69 (br d, J = 12.84 Hz, 1 H), 1.54 (m, 2 H), 1.28 (m, 2 H), 1.11 (m, 2 H). LC/MS: m/z [M + H]⁺ 460.3, Rt (I = 220 nm): 2.35 min (LC/MS method A), calc. m/z: 460.92. |
| 22 | 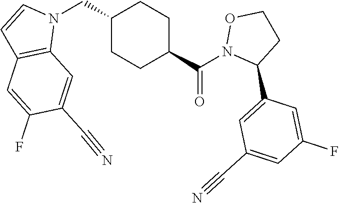 [SMILES: N#Cc1cc(F)cc([C@@H]2CCON2C(=O)[C@H]2CC[C@H](Cn3ccc4cc(F)c(C#N)cc43)CC2)c1] | ¹H NMR (400 MHz, DMSO-d₆): δ ppm 8.24 (d, J = 5.26 Hz, 1 H), 7.74 (m, 2 H), 7.59 (d, J = 10.42 Hz, 2 H), 7.45 (br d, J = 9.54 Hz, 1 H), 6.58 (d, J = 2.81 Hz, 1 H), 5.35 (m, 1 H), 4.25 (td, J = 7.70, 7.70, 2.69 Hz, 1 H), 4.11 (d, J = 7.21 Hz, 2 H), 3.86 (q, J = 8.03, 8.03, 8.03 Hz, 1 H), 2.87 (m, 1 H), 2.67 (br s, 1 H), 2.21 (m, 1 H), 1.86 (m, 2 H), 1.70 (br d, J = 12.35 Hz, 1 H), 1.53 (br t, J = 13.51, 13.51 Hz, 2 H), 1.27 (br t, J = 12.10, 12.10 Hz, 2 H), 1.10 (m, 2 H). LC/MS: m/z [M + H]⁺ 475.3, Rt (I = 220 nm): 2.6 min (LC/MS method A), calc. m/z: 475.51. |
| 23 | 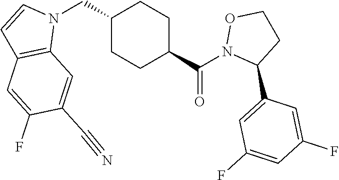 [SMILES: N#Cc1cc2c(ccn2C[C@H]2CC[C@H](C(=O)N3OCC[C@H]3c3cc(F)cc(F)c3)CC2)cc1F] | ¹H NMR (400 MHz, DMSO-d₆): δ ppm 8.24 (d, J = 5.26 Hz, 1 H), 7.74 (d, J = 2.93 Hz, 1 H), 7.59 (d, J = 10.39 Hz, 1 H), 7.12 (m, 1 H), 6.96 (br d, J = 6.60 Hz, 2 H), 6.58 (d, J = 2.93 Hz, 1 H), 5.31 (m, 1 H), 4.23 (td, J = 7.70, 7.70, 2.81 Hz, 1 H), 4.11 (d, J = 7.21 Hz, 2 H), 3.84 (m, 1 H), 2.85 (m, 1 H), 2.67 (m, 1 H), 2.17 (m, 1 H), 1.84 (m, 2 H), 1.69 (br d, J = 11.49 Hz, 1 H), 1.53 (br t, J = 13.39, 13.39 Hz, 2 H), 1.27 (m, 2 H), 1.10 (m, 2 H). LC/MS: m/z [M + H]⁺ 468.3, Rt (I = 220 nm): 2.72 min (LC/MS method A), calc. m/z: 468.49. |
| 24 | 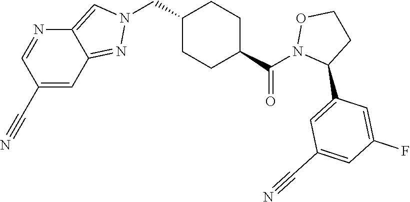 [SMILES: N#Cc1cc(F)cc([C@@H]2CCON2C(=O)[C@H]2CC[C@H](Cn3cc4ncc(C#N)cc4n3)CC2)c1] | 1H NMR (400.23 MHz, DMSO-d6) d ppm 8.90 (s, 1 H), 8.89 (s, 1 H), 8.76 (d, J = 1.83 Hz, 1 H), 7.75 (d, J = 7.91 Hz, 1 H), 7.58 (s, 1 H), 7.46 (br d, J = 9.54 Hz, 1 H), 5.35 (dd, J = 8.56, 6.60 Hz, 1 H), 4.42 (d, J = 7.09 Hz, 2 H), 4.25 (td, J = 7.76, 7.76, 2.93 Hz, 1 H), 3.86 (m, 1 H), 2.87 (dddd, J = 12.15, 9.15, 6.51, 3.12 Hz, 1 H), 2.67 (br dd, J = 3.73, 1.90 Hz, 1 H), 2.21 (m, 1 H), 2.02 (m, 1 H), 1.91 (br d, J = 11.86 Hz, 1 H), 1.72 (br d, J = 12.72 Hz, 1 H), 1.59 (br t, J = 8.25, 8.25 Hz, 2 H), 1.31 (m, 2 H), 1.15 (m, 2H). LC/MS: m/z [M + H]⁺ 459.3, Rt (I = 220 nm): 2.03 min (LC/MS method A), calc. m/z: 449.50. |
| 25 | 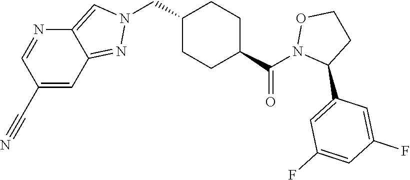 [SMILES: N#Cc1cnc2cn(C[C@H]3CC[C@H](C(=O)N4OCC[C@H]4c4cc(F)cc(F)c4)CC3)nc2c1] | 1H NMR (400.23 MHz, DMSO-d6) d ppm 8.90 (s, 1 H), 8.89 (s, 1 H), 8.76 (d, J = 1.71 Hz, 1 H), 7.12 (tt, J = 9.35, 9.35, 2.32, 2.32 Hz, 1 H), 6.96 (m, 2 H), 5.31 (dd, J = 8.50, 6.42 Hz, 1 H), 4.42 (d, J = 6.97 Hz, 2 H), 4.23 (td, J = 7.76, 7.76, 2.93 Hz, 1 H), 3.84 (m, 1 H), 2.85 (dddd, J = 12.06, 9.12, 6.51, 3.12 Hz, 1 H), 2.67 (m, 1 H), 2.17 (m, 1 H), 2.01 (td, J = 7.37, 7.37, 3.85 Hz, 1 H), 1.90 (br d, J = 12.23 Hz, 1 H), 1.70 (br d, J = 11.98 Hz, 1 H), 1.59 (br t, J = 9.48, 9.48 Hz, 2 H), 1.31 (m, 2H). LC/MS: m/z [M + H]⁺ 452.2, Rt (I = 220 nm): 2.26 (LC/MS method A), calc. m/z: 452.48. |
| 26 | 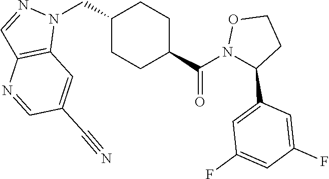 [SMILES: N#Cc1cnc2cnn(C[C@H]3CC[C@H](C(=O)N4OCC[C@H]4c4cc(F)cc(F)c4)CC3)c2c1] | ¹H NMR (400 MHz, DMSO-d₆): δ ppm 9.01 (s, 1 H), 8.85 (d, J = 1.71 Hz, 1 H), 8.50 (d, J = 0.73 Hz, 1 H), 7.11 (tt, J = 9.29, 9.29, 2.32, 2.32 Hz, 1 H), 6.95 (m, 2 H), 5.31 (dd, J = 8.56, 6.36 Hz, 1 H), 4.38 (d, J = 6.97 Hz, 2 H), 4.22 (td, J = 7.76, 7.76, 3.06 Hz, 1 H), 3.83 (m, 1 H), 2.85 (m, 1 H), 2.67 (m, 1 H), 2.17 (m, 1 H), 1.94 (m, 2 H), 1.68 (br d, J = 11.74 Hz, 1 H), 1.54 (br t, J = 13.82, 13.82 Hz, 2 H), 1.28 (m, 2 H), 1.14 (m, 2 H). LC/MS: m/z [M + H]⁺ 452.2, Rt (I = 220 nm): 2.26 min (LC/MS method A), calc. m/z: 452.48. |
| 27 | 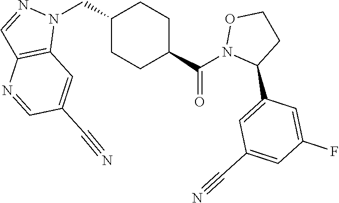 [SMILES: N#Cc1cc(F)cc([C@@H]2CCON2C(=O)[C@H]2CC[C@H](Cn3ncc4ncc(C#N)cc43)CC2)c1] | ¹H NMR (400 MHz, DMSO-d₆): δ ppm 9.01 (s, 1 H), 8.85 (d, J = 1.59 Hz, 1 H), 8.50 (d, J = 0.86 Hz, 1 H), 7.75 (d, J = 8.67 Hz, 2 H), 7.58 (s, 1 H), 7.45 (br d, J = 9.66 Hz, 1 H), 5.35 (dd, J = 8.50, 6.42 Hz, 1 H), 4.38 (d, J = 7.09 Hz, 2 H), 4.24 (td, J = 7.79, 7.79, 3.00 Hz, 1 H), 3.85 (m, 1 H), 2.87 (m, 1 H), 2.66 (m, 1 H), 2.21 (m, 1 H), 1.91 (m, 2 H), 1.69 (br d, J = 12.10 Hz, 1 H), 1.55 (br t, J = 13.33, 13.33 Hz, 2 H), 1.28 (m, 2 H), 1.14 (m, 2 H). LC/MS: m/z [M + H]⁺ 459.2, Rt (I = 220 nm): 2.13 min (LC/MS method A), calc. m/z: 459.50. |
| 28 | 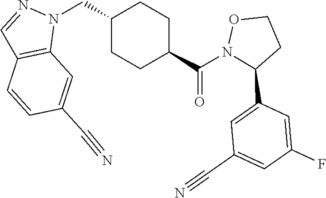 [SMILES: N#Cc1cc(F)cc([C@@H]2CCON2C(=O)[C@H]2CC[C@H](Cn3ncc4ccc(C#N)cc43)CC2)c1] | 1H NMR (DMSO-d6) δ: 8.45 (s, 1H), 8.25 (s, 1H), 7.96 (d, J = 8.3 Hz, 1H), 7.75 (br d, J = 8.4 Hz, 1H), 7.57 (s, 1H), 7.44 (d, J = 9.1 Hz, 2H), 5.31-5.37 (m, 1H), 4.35 (d, J = 7.1 Hz, 2H), 4.24 (td, J = 7.8, 3.0 Hz, 1H), 3.81-3.88 (m, 1H), 2.86 (dddd, J = 12.1, 9.1, 6.4, 3.0 Hz, 1H), 2.60-2.72 (m, 1H), 2.15-2.25 (m, 1H), 1.83- 1.96 (m, 2H), 1.68 (br d, J = 12.5 Hz, 1H), 1.53 (br t, J = 14.2 Hz, 2H), 1.06-1.33 (m, 4H). LC/MS: m/z [M + H]⁺ 458.2, Rt (I = 220 nm): 2.36 min (LC/MS method A), calc. m/z: 458.51. |
| 29 | 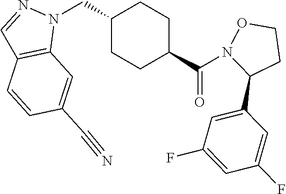 [SMILES: N#Cc1ccc2cnn(C[C@H]3CC[C@H](C(=O)N4OCC[C@H]4c4cc(F)cc(F)c4)CC3)c2c1] | 1H NMR (DMSO-d6) δ: 8.47 (s, 1H), 8.26 (s, 1H), 7.98 (d, J = 8.3 Hz, 1H), 7.46 (dd, J = 8.3, 1.1 Hz, 1H), 7.12 (tt, J = 9.3, 2.3 Hz, 1H), 6.93-6.99 (m, 2H), 5.31 (dd, J = 8.3, 6.6 Hz, 1H), 4.36 (d, J = 7.1 Hz, 2H), 4.23 (td, J = 7.7, 3.0 Hz, 1H), 3.81-3.87 (m, 1H), 2.85 (dddd, J = 12.1, 9.1, 6.5, 3.1 Hz, 1H), 2.63-2.72 (m, 1H), 2.12- 2.23 (m, 1H), 1.84-1.96 (m, 2H), 1.68 (br d, J = 11.4 Hz, 1H), 1.55 (br t, J = 14.4 Hz, 2H) 1.06-1.34 (m, 4H), 0.01 (s, 1H). LC/MS: m/z [M + H]⁺ 451.2, Rt (I = 220 nm): 2.49 min (LC/MS method A), calc. m/z: 451.49. |
| 30 | 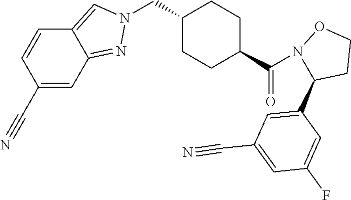 [SMILES: N#Cc1cc(F)cc([C@@H]2CCON2C(=O)[C@H]2CC[C@H](Cn3cc4ccc(C#N)cc4n3)CC2)c1] | 1H NMR (DMSO-d6) δ: 8.54 (s, 1H), 8.30 (s, 1H), 7.91 (d, J = 8.7 Hz, 1H), 7.75 (d, J = 7.9 Hz, 1H), 7.57 (s, 1H), 7.44 (br d, J = 9.7 Hz, 1H), 7.27 (dd, J = 8.7, 1.2 Hz, 1H), 5.34 (dd, J = 8.4, 6.6 Hz, 1H), 4.36 (d, J = 7.0 Hz, 2H), 4.23 (td, J = 7.8, 3.0 Hz, 1H), 3.81-3.88 (m, 1H), 2.86 (dddd, J = 12.1, 9.2, 6.5, 3.1 Hz, 1H), 2.60- 2.72 (m, 1H), 2.13-2.26 (m, 1H), 1.86-2.03 (m, 2H), 1.70 (br d, J = 12.2 Hz, 1H), 1.57 (br t, J = 8.9 Hz, 2H), 1.23-1.37 (m, 2H), 1.03-1.21 (m, 2H). LC/MS: m/z [M + H]⁺ 458.2, Rt (I = 220 nm): 2.24 min (LC/MS method A), calc. m/z: 458.51. |
| 31 | 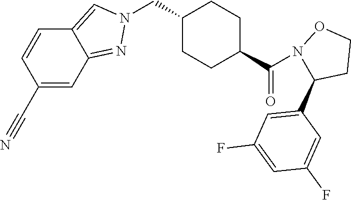 [SMILES: N#Cc1ccc2cn(C[C@H]3CC[C@H](C(=O)N4OCC[C@H]4c4cc(F)cc(F)c4)CC3)nc2c1] | 1H NMR (DMSO-d6) δ: 8.56 (s, 1H), 8.31 (s, 1H), 7.92 (d, J = 8.6 Hz, 1H), 7.28 (dd, J = 8.6, 1.2 Hz, 1H), 7.12 (tt, J = 9.4, 2.3 Hz, 1H), 6.93-6.99 (m, 2H), 5.31 (dd, J = 8.6, 6.5 Hz, 1H), 4.38 (d, J = 7.1 Hz, 2H), 4.23 (td, J = 7.7, 3.0 Hz, 1H), 3.80-3.88 (m, 1H), 2.85 (dddd, J = 12.1, 9.1, 6.5, 3.1 Hz, 1H), 2.62-2.73 (m, 1H), 2.12- 2.23 (m, 1H), 1.87-2.03 (m, 2H), 1.70 (br d, J = 11.4 Hz, 1H), 1.58 (br t, J = 11.7 Hz, 2H), 1.24-1.38 (m, 2H), 1.03-1.21 (m, 2H), 0.00 (s, 1H). LC/MS: m/z [M + H]⁺ 451.2, Rt (I = 220 nm): 2.37 min (LC/MS method A), calc. m/z: 451.49. |
| 32 | 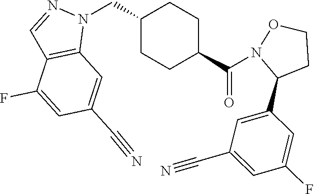 [SMILES: N#Cc1cc(F)cc([C@@H]2CCON2C(=O)[C@H]2CC[C@H](Cn3ncc4c(F)cc(C#N)cc43)CC2)c1] | 1H NMR (DMSO-d6) δ: 8.38 (d, J = 2.4 Hz, 2H), 7.75 (dm, J - 1.22 Hz, 1H), 7.57 (m, 1H), 7.44 (m, 2H), 5.35 (m, 1H), 4.37 (d, J = 7.0 Hz, 4H), 4.24 (td, J, 7.76, 2.93 Hz, 2H), 3.86 (m, 2H), 2.87 (dddd, J = 12.2, 9.12, 6.45, 3.12, 2H), 2.67 (m, 2H), 2.21 (m, 2H), 1.89 (br d, J = 12.59, 2H), 1.69 (bd, J = 12.59, 1H), 1.55 (bt, J = 13.3, 2H), 1.22-1.05 (m, 2H). LC/MS: m/z [M + H]⁺ 476.2, Rt (I = 220 nm): 2.51 min (LC/MS method A), calc. m/z: 476.50. |
| 33 | 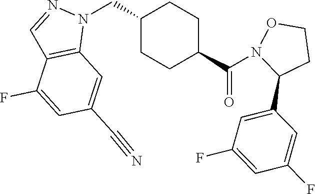 [SMILES: N#Cc1cc(F)c2cnn(C[C@H]3CC[C@H](C(=O)N4OCC[C@H]4c4cc(F)cc(F)c4)CC3)c2c1] | 1H NMR (DMSO-d6) δ: 8.37-8.41 (m, 2H), 7.43 (d, J = 9.9 Hz, 1H), 7.11 (tt, J = 9.3, 2.2 Hz, 1H), 6.95 (br d, J = 6.6 Hz, 2H), 5.31 (dd, J = 8.3, 6.5 Hz, 1H), 4.38 (d, J = 7.0 Hz, 2H), 4.18-4.26 (m, 1H), 3.79-3.87 (m, 1H), 2.85 (dddd, J = 12.1, 9.1, 6.5, 3.0 Hz, 1H), 2.62-2.72 (m, 1H), 2.12-2.22 (m, 1H), 1.83-1.96 (m, 2H), 1.68 (br d, J = 11.4 Hz, 1H), 1.54 (br t, J = 13.6 Hz, 2H), 1.06- 1.34 (m, 4H). LC/MS: m/z [M + H]⁺ 469.2, Rt (I = 220 nm): 2.64 min (LC/MS method A), calc. m/z: 469.48. |
| 34 | 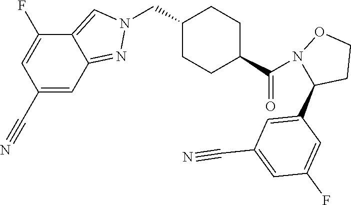 [SMILES: N#Cc1cc(F)cc([C@@H]2CCON2C(=O)[C@H]2CC[C@H](Cn3cc4c(F)cc(C#N)cc4n3)CC2)c1] | 1H NMR (DMSO-d6) δ: 8.76 (s, 1H), 8.22 (s, 1H), 7.74 (br d, J = 7.9 Hz, 1H), 7.57 (s, 1H), 7.44 (br d, J = 9.7 Hz, 1H), 7.21 (d, J = 10.3 Hz, 1H), 5.34 (dd, J = 8.6, 6.5 Hz, 1H), 4.37 (d, J = 7.1 Hz, 2H), 4.23 (td, J = 7.8, 3.0 Hz, 1H), 3.81-3.88 (m, 1H), 2.85 (dddd, J = 12.2, 9.1, 6.4, 3.1 Hz, 1H), 2.58-2.73 (m, 1H), 2.14- 2.25 (m, 1H), 2.06 (s, 1H), 1.85-2.01 (m, 2H), 1.70 (br d, J = 12.3 Hz, 1H), 1.51-1.63 (m, 2H), 1.22-1.37 (m, 2H), 1.02-1.19 (m, 2H). LC/MS: m/z [M + H]⁺ 476.3, Rt (I = 220 nm): 2.38 min (LC/MS method A), calc. m/z: 476.50. |
| 35 | 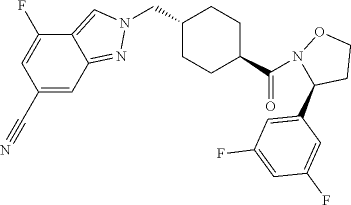 [SMILES: N#Cc1cc(F)c2cn(C[C@H]3CC[C@H](C(=O)N4OCC[C@H]4c4cc(F)cc(F)c4)CC3)nc2c1] | 1H NMR (DMSO-d6) δ: 8.76 (s, 1H), 8.21 (s, 1H), 7.21 (d, J = 10.3 Hz, 1H), 7.09 (tt, J = 9.3, 2.3 Hz, 1H), 6.90-6.97 (m, 2H), 5.29 (dd, J = 8.4, 6.5 Hz, 1H), 4.37 (d, J = 7.1 Hz, 2H), 4.21 (td, J = 7.7, 3.0 Hz, 1H), 3.78- 3.86 (m, 1H), 2.83 (dddd, J = 12.1, 9.1, 6.5, 3.1 Hz, 1H), 2.60-2.70 (m, 1H), 2.10-2.20 (m, 1H), 1.98 (dt, J = 7.0, 3.7 Hz, 1H), 1.88 (br d, J = 12.2 Hz, 1H), 1.68 (br d, J = 11.4 Hz, 1H), 1.57 (br t, J = 12.2 Hz, 2H), 1.22- 1.36 (m, 2H), 1.01-1.19 (m, 2H). LC/MS: m/z [M + H]⁺ 469.2, Rt (I = 220 nm): 2.513 min (LC/MS method A), calc. m/z: 469.48. |
| 36 | 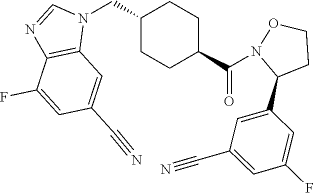 [SMILES: N#Cc1cc(F)cc([C@@H]2CCON2C(=O)[C@H]2CC[C@H](Cn3cnc4c(F)cc(C#N)cc43)CC2)c1] | 1H NMR (400.23 MHz, DMSO-d6) d ppm 8.55 (s, 1 H), 8.27 (d, J = 1.10 Hz, 1 H), 7.75 (br d, J = 8.56 Hz, 1 H), 7.59 (d, J = 10.37 Hz, 1 H), 7.58 (s, 1 H), 7.46 (br d, J = 9.78 Hz, 1 H), 5.35 (m, 1 H), 4.25 (td, J = 7.86, 7.86, 3.00 Hz, 1 H), 4.20 (d, J = 7.21 Hz, 2 H), 3.86 (m, 1 H), 2.87 (dddd, J = 12.13, 9.11, 6.39, 2.93 Hz, 1 H), 2.67 (br d, J = 1.96 Hz, 1 H), 2.21 (m, 1 H), 1.89 (br d, J = 11.86 Hz, 2 H), 1.71 (br d, J = 11.62 Hz, 1 H), 1.58 (m, 2 H), 1.29 (m, 2 H), 1.11 (m, 2 H). LC/MS: m/z [M + H]⁺ 476.3, Rt (I = 220 nm): 2.13 min (LC/MS method A), calc. m/z: 476.50. |
| 37 | 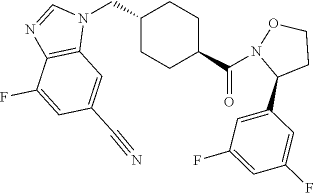 [SMILES: N#Cc1cc(F)c2ncn(C[C@H]3CC[C@H](C(=O)N4OCC[C@H]4c4cc(F)cc(F)c4)CC3)c2c1] | ¹H NMR (400 MHz, DMSO-d₆): δ ppm 8.55 (s, 1 H), 8.27 (d, J = 1.10 Hz, 1 H), 7.59 (dd, J = 10.64, 0.98 Hz, 1 H), 7.12 (tt, J = 9.32, 9.32, 2.29, 2.29 Hz, 1 H), 6.96 (br d, J = 6.60 Hz, 2 H), 5.31 (m, 1 H), 4.21 (m, 3 H), 3.84 (m, 1 H), 2.85 (m, 1 H), 2.67 (br s, 1 H), 2.18 (m, 1 H), 1.89 (br d, J = 8.56 Hz, 2 H), 1.70 (br d, J = 11.62 Hz, 1 H), 1.57 (br t, J = 12.29, 12.29 Hz, 2 H), 1.29 (m, 2 H), 1.12 (m, 2 H). LC/MS: m/z [M + H]⁺ 469.3, Rt (I = 220 nm): 2.26 min (LC/MS method A), calc. m/z: 469.48. |
| 38 | 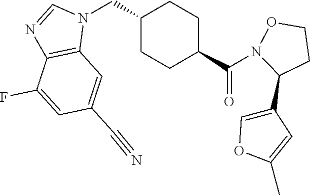 [SMILES: Cc1cc([C@@H]2CCON2C(=O)[C@H]2CC[C@H](Cn3cnc4c(F)cc(C#N)cc43)CC2)co1] | 1H NMR (400.23 MHz, DMSO-d6) d ppm 8.54 (s, 1 H), 8.26 (d, J = 1.10 Hz, 1 H), 7.59 (d, J = 10.31 Hz, 1 H), 7.38 (s, 1 H), 6.01 (s, 1 H), 5.17 (dd, J = 8.19, 5.50 Hz, 1 H), 4.19 (br d, J = 7.09 Hz, 3 H), 3.80 (q, J = 7.91, 7.91, 7.91 Hz, 1 H), 2.62 (m, 2 H), 2.22 (m, 1 H), 2.20 (s, 3 H), 1.86 (br s, 1 H), 1.79 (br d, J = 12.72 Hz, 1 H), 1.67 (br d, J = 11.74 Hz, 1 H), 1.55 (br d, J = 12.10 Hz, 2 H), 1.27 (m, 2 H), 1.08 (m, 2 H). LC/MS: m/z [M + H]⁺ 437.3, Rt (I = 220 nm): 2.09 min (LC/MS method A), calc. m/z: 437.49. |
| 39 | 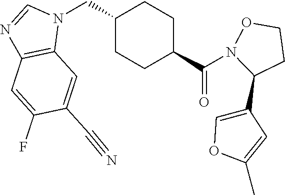 [SMILES: Cc1cc([C@@H]2CCON2C(=O)[C@H]2CC[C@H](Cn3cnc4cc(F)c(C#N)cc43)CC2)co1] | 1H NMR (400.23 MHz, DMSO-d6) d ppm 8.56 (s, 1 H), 8.43 (d, J = 5.62 Hz, 1 H), 7.79 (d, J = 10.27 Hz, 1 H), 7.38 (s, 1 H), 6.01 (s, 1 H), 5.17 (dd, J = 8.44, 5.50 Hz, 1 H), 4.17 (m, 3 H), 3.80 (q, J = 7.74, 7.74, 7.74 Hz, 1 H), 2.61 (m, 2 H), 2.22 (m, 1 H), 2.20 (s, 4 H), 1.83 (m, 2 H), 1.67 (br d, J = 12.23 Hz, 1 H), 1.55 (br d, J = 12.35 Hz, 2 H), 1.28 (m, 2 H), 1.08 (m, 2 H). LC/MS: m/z [M + H]⁺ 437.3, Rt (I = 220 nm): 2.08 min (LC/MS method A), calc. m/z: 437.49. |
| 40 | 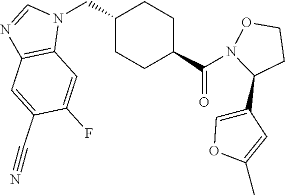 [SMILES: Cc1cc([C@@H]2CCON2C(=O)[C@H]2CC[C@H](Cn3cnc4cc(C#N)c(F)cc43)CC2)co1] | 1H NMR (400.23 MHz, DMSO-d6) d ppm 8.48 (s, 1 H), 8.28 (d, J = 5.75 Hz, 1 H), 7.95 (d, J = 9.90 Hz, 1 H), 7.38 (s, 1 H), 6.01 (s, 1 H), 5.17 (dd, J = 8.31, 5.62 Hz, 1 H), 4.15 (m, 3 H), 3.79 (q, J = 7.87, 7.87, 7.87 Hz, 1 H), 2.61 (m, 2 H), 2.22 (m, 1 H), 2.19 (s, 4 H), 1.81 (m, 2 H), 1.66 (br d, J = 12.35 Hz, 1 H), 1.55 (br d, J = 12.23 Hz, 2 H), 1.27 (m, 2 H), 1.08 (m, 2 H). LC/MS: m/z [M + H]⁺ 437.3, Rt (I = 220 nm): 2.09 min (LC/MS method A), calc. m/z: 437.49. |
| 41 | 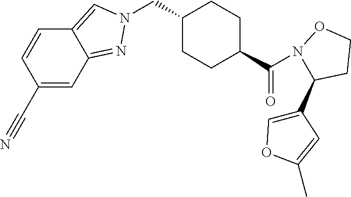 [SMILES: Cc1cc([C@@H]2CCON2C(=O)[C@H]2CC[C@H](Cn3cc4ccc(C#N)cc4n3)CC2)co1] | 1H NMR (400.23 MHz, DMSO-d6) d ppm 8.55 (s, 1 H), 8.31 (s, 1 H), 7.92 (d, J = 8.68 Hz, 1 H), 7.38 (s, 1 H), 7.28 (dd, J = 8.62, 1.16 Hz, 1 H), 6.01 (s, 1 H), 5.17 (dd, J = 8.44, 5.62 Hz, 1 H), 4.36 (d, J = 6.97 Hz, 2 H), 4.18 (td, J = 7.70, 7.70, 3.79 Hz, 1 H), 3.79 (q, J = 7.87, 7.87, 7.87 Hz, 1 H), 2.61 (m, 2 H), 2.22 (m, 1 H), 2.19 (s, 3 H), 1.96 (td, J = 7.40, 7.40, 3.79 Hz, 1 H), 1.80 (br d, J = 12.23 Hz, 1 H), 1.67 (br d, J = 11.37 Hz, 1 H), 1.56 (br d, J = 10.88 Hz, 2 H), 1.29 (q, J = 12.76, 12.76, 12.76 Hz, 2 H. LC/MS: m/z [M + H]⁺ 419.3, Rt (I = 220 nm): 2.2 min (LC/MS method A), calc. m/z: 419.50. |
| 42 | 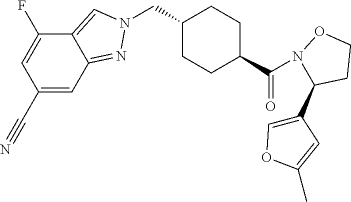 [SMILES: Cc1cc([C@@H]2CCON2C(=O)[C@H]2CC[C@H](Cn3cc4c(F)cc(C#N)cc4n3)CC2)co1] | 1H NMR (400.23 MHz, DMSO-d6) d ppm 8.77 (s, 1 H), 8.23 (s, 1 H), 7.38 (s, 1 H), 7.23 (d, J = 10.27 Hz, 1 H), 6.01 (s, 1 H), 5.17 (dd, J = 8.38, 5.56 Hz, 1 H), 4.38 (d, J = 6.97 Hz, 2 H), 4.18 (td, J = 7.61, 7.61, 3.85 Hz, 1 H), 3.80 (q, J = 7.87, 7.87, 7.87 Hz, 1 H), 2.61 (m, 2 H), 2.22 (m, 1 H), 2.20 (s, 4 H), 1.98 (m, 1 H), 1.80 (br d, J = 11.49 Hz, 1 H), 1.67 (br d, J = 12.35 Hz, 1 H), 1.56 (br d, J = 11.0 Hz, 2 H), 1.29 (q, J = 12.31, 12.31, 12.31 Hz, 2 H), 1.10 (m, 2 H). LC/MS: m/z [M + H]⁺ 437.3, Rt (I = 220 nm): 2.34 min (LC/MS method A), calc. m/z: 437.49. |
| 43 | 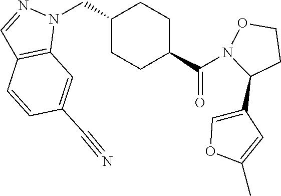 [SMILES: Cc1cc([C@@H]2CCON2C(=O)[C@H]2CC[C@H](Cn3ncc4ccc(C#N)cc43)CC2)co1] | ¹H NMR (400 MHz, DMSO-d₆): δ ppm 8.45 (s, 1 H), 8.25 (s, 1 H), 7.97 (d, J = 8.31 Hz, 1 H), 7.45 (d, J = 7.96 Hz, 1 H), 7.37 (s, 1 H), 6.01 (s, 1 H), 5.17 (dd, J = 8.44, 5.62 Hz, 1 H), 4.35 (d, J = 6.97 Hz, 2 H), 4.18 (td, J = 7.73, 7.73, 3.85 Hz, 1 H), 3.79 (q, J = 7.82, 7.82, 7.82 Hz, 1 H), 2.61 (m, 2 H), 2.19 (s, 3 H), 2.19 (m, 1 H), 1.89 (br d, J = 8.31 Hz, 1 H), 1.77 (br d, J = 12.72 Hz, 1 H), 1.65 (br d, J = 12.96 Hz, 1 H), 1.52 (br d, J = 12.35 Hz, 2 H), 1.26 (m, 2 H), 1.11 (m, 2 H). LC/MS: m/z [M + H]⁺ 419.3, Rt (I = 220 nm): 2.32 min (LC/MS method A), calc. m/z: 419.50. |
| 44 | 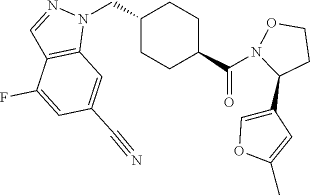 [SMILES: Cc1cc([C@@H]2CCON2C(=O)[C@H]2CC[C@H](Cn3ncc4c(F)cc(C#N)cc43)CC2)co1] | ¹H NMR (400 MHz, DMSO-d₆): δ ppm 8.39 (s, 2 H), 7.42 (d, J = 9.90 Hz, 1 H), 7.37 (s, 1 H), 6.01 (s, 1 H), 5.17 (dd, J = 8.25, 5.56 Hz, 1 H), 4.37 (d, J = 6.97 Hz, 2 H), 4.18 (td, J = 7.58, 7.58, 3.79 Hz, 1 H), 3.79 (q, J = 7.91, 7.91, 7.91 Hz, 1 H), 2.61 (m, 2 H), 2.33 (s, 3 H), 2.20 (m, 1 H), 1.89 (br d, J = 8.19 Hz, 1 H), 1.78 (br d, J = 12.35 Hz, 1 H), 1.65 (br d, J = 12.96 Hz, 1 H), 1.52 (m, 3 H), 1.27 (m, 2 H), 1.11 (m, 2H). LC/MS: m/z [M + H]⁺ 437.3, Rt (I = 220 nm): 2.47 min (LC/MS method A), calc. m/z: 437.49. |
| 45 | 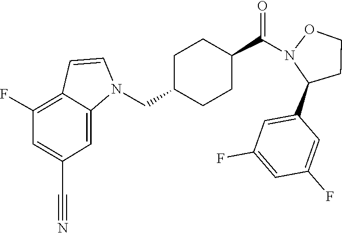 [SMILES: N#Cc1cc(F)c2ccn(C[C@H]3CC[C@H](C(=O)N4OCC[C@H]4c4cc(F)cc(F)c4)CC3)c2c1] | ¹H NMR (400 MHz, DMSO-d₆): δ ppm 8.12 (s, 1 H), 7.72 (d, J = 3.18 Hz, 1 H), 7.27 (d, J = 10.27 Hz, 1 H), 7.11 (m, 1 H), 6.96 (br d, J = 6.72 Hz, 2 H), 6.67 (d, J = 2.93 Hz, 1 H), 5.31 (m, 1 H), 4.23 (td, J = 7.73, 7.73, 3.00 Hz, 1 H), 4.14 (d, J = 7.09 Hz, 2 H), 3.83 (m, 1 H), 2.85 (m, 1 H), 2.67 (br s, 1 H), 2.18 (m, 1 H), 1.86 (br t, J = 11.49, 11.49 Hz, 2 H), 1.68 (br d, J = 12.72 Hz, 1 H), 1.53 (m, 2 H), 1.27 (m, 2 H), 1.11 (m, 2 H). LC/MS: m/z [M + H]⁺ 468.3, Rt (I = 220 nm): 2.76 min (LC/MS method A), calc. m/z: 468.50. |
| 46 | 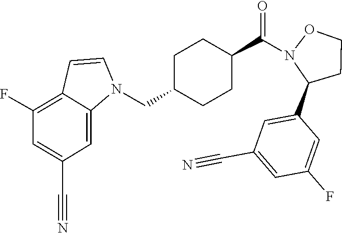 [SMILES: N#Cc1cc(F)cc([C@@H]2CCON2C(=O)[C@H]2CC[C@H](Cn3ccc4c(F)cc(C#N)cc43)CC2)c1] | ¹H NMR (400 MHz, DMSO-d₆): δ ppm 8.12 (s, 1 H), 7.75 (m, 2 H), 7.58 (s, 1 H), 7.45 (br d, J = 9.54 Hz, 1 H), 7.28 (d, J = 10.15 Hz, 1 H), 6.67 (d, J = 2.93 Hz, 1 H), 5.35 (m, 1 H), 4.25 (td, J = 7.73, 7.73, 2.75 Hz, 1 H), 4.15 (d, J = 7.21 Hz, 2 H), 3.86 (m, 1 H), 2.87 (m, 1 H), 2.67 (br s, 1 H), 2.21 (m, 1 H), 1.85 (m, 2 H), 1.70 (br d, J = 11.62 Hz, 1 H), 1.54 (br t, J = 13.88, 13.88 Hz, 2 H), 1.27 (m, 2 H), 1.12 (m, 2 H). LC/MS: m/z [M + H]⁺ 475.4, Rt (I = 220 nm): 2.64 min (LC/MS method A), calc. m/z: 475.51. |
| 47 | 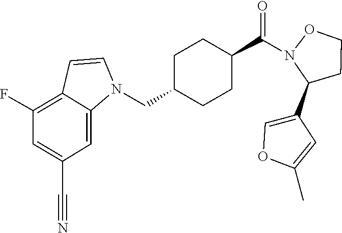 [SMILES: Cc1cc([C@@H]2CCON2C(=O)[C@H]2CC[C@H](Cn3ccc4c(F)cc(C#N)cc43)CC2)co1] | ¹H NMR (400 MHz, DMSO-d₆): δ ppm 8.11 (s, 1 H), 7.71 (d, J = 3.18 Hz, 1 H), 7.38 (s, 1 H), 7.27 (d, J = 10.27 Hz, 1 H), 6.66 (d, J = 3.06 Hz, 1 H), 6.01 (s, 1 H), 5.17 (dd, J = 8.31, 5.62 Hz, 1 H), 4.18 (m, 1 H), 4.13 (d, J = 7.21 Hz, 2 H), 3.79 (q, J = 7.87, 7.87, 7.87 Hz, 1 H), 2.61 (m, 2 H), 2.22 (m, 1 H), 2.19 (s, 3 H), 1.79 (m, 2 H), 1.65 (br d, J = 11.62 Hz, 1 H), 1.51 (br d, J = 12.23 Hz, 2 H), 1.26 (m, 2 H), 1.08 (m, 2 H). LC/MS: m/z [M + H]⁺ 436.3, Rt (I = 220 nm): 2.61 min (LC/MS method A), calc. m/z: 436.50. |
| 48 | 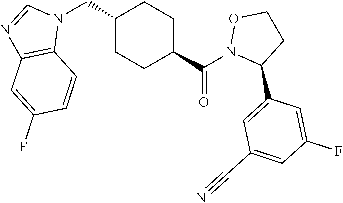 [SMILES: N#Cc1cc(F)cc([C@@H]2CCON2C(=O)[C@H]2CC[C@H](Cn3cnc4cc(F)ccc43)CC2)c1] | 1H NMR (400.23 MHz, DMSO-d6) d ppm 8.85 (br s, 1 H), 7.87 (dd, J = 8.93, 4.65 Hz, 1 H), 7.75 (d, J = 8.64 Hz, 1 H), 7.60 (br d, J = 2.45 Hz, 1 H), 7.58 (s, 1 H), 7.45 (br d, J = 9.54 Hz, 1 H), 7.32 (td, J = 9.29, 9.29, 2.32 Hz, 1 H), 5.35 (dd, J = 8.44, 6.60 Hz, 1 H), 4.24 (m, 3 H), 3.86 (m, 1 H), 2.87 (dddd, J = 12.07, 9.11, 6.39, 3.12 Hz, 2 H), 2.67 (m, 2 H), 2.33 (m, 1 H), 2.21 (m, 2 H), 1.90 (br d, J = 11.98 Hz, 2 H), 1.71 (br d, J = 10.88 Hz, 1 H), 1.62 (m, 2 H), 1.29 (m, 2 H), 1.14 (m, 2 H). LC/MS: m/z [M + H]⁺ 451.3, Rt (I = 220 nm): 1.77 min (LC/MS method A), calc. m/z: 451.49. |
| 49 | 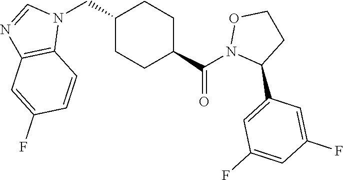 [SMILES: O=C([C@H]1CC[C@H](Cn2cnc3cc(F)ccc32)CC1)N1OCC[C@H]1c1cc(F)cc(F)c1] | 1H NMR (400.23 MHz, DMSO-d6) d ppm 8.90 (s, 1 H), 7.89 (dd, J = 8.99, 4.58 Hz, 1 H), 7.60 (dd, J = 9.17, 2.45 Hz, 1 H), 7.33 (td, J = 9.29, 9.29, 2.45 Hz, 1 H), 7.12 (tt, J = 9.35, 9.35, 2.32, 2.32 Hz, 1 H), 6.96 (m, 2 H), 5.31 (dd, J = 8.38, 6.42 Hz, 1 H), 4.24 (br d, J = 7.21 Hz, 3 H), 4.21 (br d, J = 3.06 Hz, 1 H), 3.84 (m, 1 H), 2.86 (dddd, J = 12.12, 9.15, 6.48, 3.06 Hz, 1 H), 2.67 (br d, J = 1.83 Hz, 1 H), 2.18 (m, 1 H), 1.90 (br d, J = 11.37 Hz, 2 H), 1.65 (m, 3 H), 1.29 (m, 2 H), 1.14 (m, 2 H). LC/MS: m/z [M + H]⁺ 444.3, Rt (I = 220 nm): 1.9 min (LC/MS method A), calc. m/z: 444.47. |
| 50 | 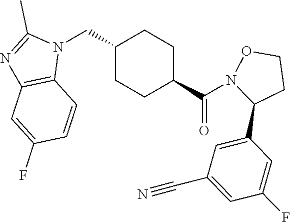 [SMILES: Cc1nc2cc(F)ccc2n1C[C@H]1CC[C@H](C(=O)N2OCC[C@H]2c2cc(F)cc(C#N)c2)CC1] | 1H NMR (420.23 MHz, DMSO-d6) d ppm 7.94 (dd, J = 8.99, 4.34 Hz, 1 H), 7.76 (d, J = 8.65 Hz, 1 H), 7.64 (dd, J = 8.80, 2.32 Hz, 1 H), 7.58 (s, 1 H), 7.46 (br d, J = 9.54 Hz, 1 H), 7.38 (td, J = 9.32, 9.32, 2.26 Hz, 1 H), 5.35 (dd, J = 8.62, 6.42 Hz, 1 H), 4.25 (m, 3 H), 3.87 (m, 1 H), 2.88 (tdd, J = 12.18, 12.18, 6.27, 2.75 Hz, 2 H), 2.76 (s, 3 H), 2.68 (m, 1 H), 2.33 (m, 1 H), 2.22 (m, 1 H), 1.90 (br d, J = 9.90 Hz, 2 H), 1.66 (m, 3 H), 1.27 (m, 4 H). LC/MS: m/z [M + H]⁺ 465.3, Rt (I = 220 nm): 1.56 min (LC/MS method A), calc. m/z: 465.52. |
| 51 | 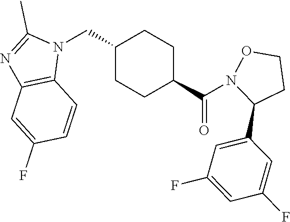 [SMILES: Cc1nc2cc(F)ccc2n1C[C@H]1CC[C@H](C(=O)N2OCC[C@H]2c2cc(F)cc(F)c2)CC1] | 1H NMR (400.23 MHz, DMSO-d6) d ppm 7.93 (dd, J = 8.99, 4.34 Hz, 1 H), 7.63 (dd, J = 8.80, 2.32 Hz, 1 H), 7.37 (td, J = 9.29, 9.29, 2.20 Hz, 1 H), 7.12 (tt, J = 9.29, 9.29, 2.32, 2.32 Hz, 1 H), 6.96 (br d, J = 6.48 Hz, 2 H), 5.31 (m, 1 H), 4.26 (br d, J = 3.18 Hz, 1 H), 4.23 (br d, J = 7.46 Hz, 3 H), 3.84 (m, 1 H), 2.86 (dddd, J = 12.10, 9.11, 6.42, 3.18 Hz, 2 H), 2.75 (s, 4 H), 2.68 (m, 1 H), 2.18 (m, 1 H), 1.89 (br d, J = 11.13 Hz, 2 H), 1.66 (m, 3 H), 1.27 (m, 4 H). LC/MS: m/z [M + H]⁺ 458.3, Rt (I = 220 nm): 1.68 min (LC/MS method A), calc. m/z: 458.50. |
| 52 | 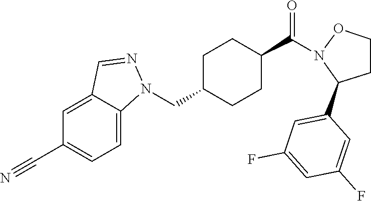 [SMILES: N#Cc1ccc2c(cnn2C[C@H]2CC[C@H](C(=O)N3OCC[C@H]3c3cc(F)cc(F)c3)CC2)c1] | ¹H NMR (400 MHz, DMSO-d₆): δ ppm 8.40 (s, 1 H), 8.27 (d, J = 0.73 Hz, 1 H), 7.92 (d, J = 8.80 Hz, 1 H), 7.71 (dd, J = 8.80, 1.47 Hz, 1 H), 7.11 (tt, J = 9.35, 9.35, 2.32, 2.32 Hz, 1 H), 6.95 (m, 2 H), 5.30 (dd, J = 8.56, 6.36 Hz, 1 H), 4.34 (d, J = 6.97 Hz, 2 H), 4.22 (td, J = 7.76, 7.76, 2.93 Hz, 1 H), 3.83 (m, 1 H), 2.85 (m, 1 H), 2.67 (m, 1 H), 2.65 (br s, 1 H), 2.17 (m, 1 H), 1.87 (m, 2 H), 1.67 (br d, J = 10.76 Hz, 1 H), 1.55 (m, 2 H), 1.27 (m, 2 H), 1.11 (m, 2H). LC/MS: m/z [M + H]⁺ 451.3, Rt (I = 220 nm): 2.44 min (LC/MS method A), calc. m/z: 451.49. |
| 53 | 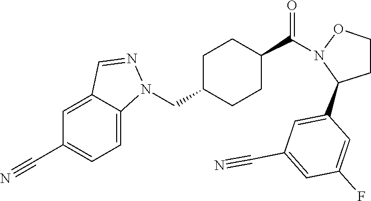 [SMILES: N#Cc1cc(F)cc([C@@H]2CCON2C(=O)[C@H]2CC[C@H](Cn3ncc4cc(C#N)ccc43)CC2)c1] | ¹H NMR (400 MHz, DMSO-d₆): δ ppm 8.40 (dd, J = 1.34, 0.73 Hz, 1 H), 8.27 (d, J = 0.86 Hz, 1 H), 7.92 (d, J = 8.80 Hz, 1 H), 7.75 (d, J = 8.71 Hz, 1 H), 7.71 (dd, J = 8.80, 1.47 Hz, 1 H), 7.58 (s, 1 H), 7.45 (d, J = 9.57 Hz, 1 H), 5.34 (dd, J = 8.56, 6.36 Hz, 1 H), 4.34 (d, J = 6.97 Hz, 2 H), 4.24 (td, J = 7.73, 7.73, 3.00 Hz, 1 H), 3.85 (m, 1 H), 2.86 (m, 1 H), 2.67 (br dd, J = 3.73, 1.90 Hz, 1 H), 2.20 (m, 1 H), 1.89 (m, 1 H), 1.86 (br s, 1 H), 1.68 (br d, J = 12.47 Hz, 1 H), 1.55 (br t, J = 14.24, 14.24 Hz, 2 H), 1.33 (m, 2 H), 1.12 (m, 2 H). LC/MS: m/z [M + H]⁺ 458.3, Rt (I = 220 nm): 2.3 min (LC/MS method A), calc. m/z: 458.51. |
| 54 | 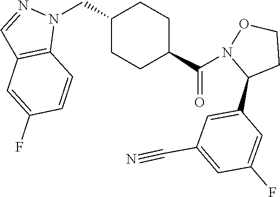 [SMILES: N#Cc1cc(F)cc([C@@H]2CCON2C(=O)[C@H]2CC[C@H](Cn3ncc4cc(F)ccc43)CC2)c1] | ¹H NMR (400 MHz, DMSO-d₆): δ ppm 8.04 (s, 1 H), 7.74 (m, 2 H), 7.52 (m, 3 H), 7.27 (td, J = 9.20, 9.20, 2.38 Hz, 1 H), 5.34 (dd, J = 8.38, 6.42 Hz, 1 H), 4.26 (m, 3 H), 3.85 (m, 1 H), 2.86 (m, 1 H), 2.67 (br s, 1 H), 2.20 (m, 1 H), 1.89 (br d, J = 3.42 Hz, 1 H), 1.86 (br s, 2 H), 1.68 (br d, J = 12.35 Hz, 1 H), 1.55 (br t, J = 14.49, 14.49 Hz, 2 H), 1.26 (m, 2 H), 1.12 (m, 2 H). LC/MS: m/z [M + H]⁺ 451.3, Rt (I = 220 nm): 2.47 min (LC/MS method A), calc. m/z: 451.49. |
| 55 | 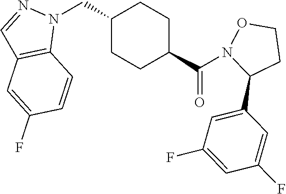 [SMILES: O=C([C@H]1CC[C@H](Cn2ncc3cc(F)ccc32)CC1)N1OCC[C@H]1c1cc(F)cc(F)c1] | ¹H NMR (400 MHz, DMSO-d₆): δ ppm 8.04 (s, 1 H), 7.74 (dd, J = 9.11, 4.22 Hz, 1 H), 7.52 (dd, J = 9.23, 2.38 Hz, 1 H), 7.27 (td, J = 9.11, 9.11, 2.45 Hz, 1 H), 7.11 (t, J = 9.30, 9.30 Hz, 1 H), 6.95 (br d, J = 6.48 Hz, 2 H), 5.31 (m, 1 H), 4.27 (d, J = 6.97 Hz, 2 H), 4.22 (td, J = 7.58, 7.58, 2.69 Hz, 1 H), 3.83 (m, 1 H), 2.85 (m, 1 H), 2.67 (br s, 1 H), 2.17 (m, 1 H), 1.87 (br d, J = 11.37 Hz, 2 H), 1.67 (br d, J = 13.82 Hz, 1 H), 1.55 (m, 2 H), 1.27 (m, 2 H), 1.12 (m, 2 H). LC/MS: m/z [M + H]⁺ 444.2, Rt (I = 220 nm): 2.59 min (LC/MS method A), calc. m/z: 444.47. |
| 56 | 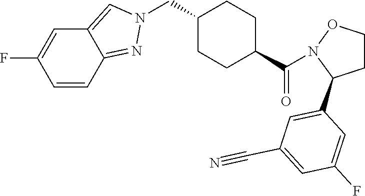 [SMILES: N#Cc1cc(F)cc([C@@H]2CCON2C(=O)[C@H]2CC[C@H](Cn3cc4cc(F)ccc4n3)CC2)c1] | 1H NMR (400.23 MHz, DMSO-d6) d ppm 8.32 (s, 1 H), 7.75 (br d, J = 8.56 Hz, 1 H), 7.66 (dd, J = 9.41, 4.89 Hz, 1 H), 7.58 (s, 1 H), 7.43 (m, 2 H), 7.12 (td, J = 9.32, 9.32, 2.51 Hz, 1 H), 5.35 (m, 1 H), 4.27 (m, 3 H), 3.86 (m, 1 H), 2.86 (br dd, J = 6.30, 2.87 Hz, 1 H), 2.67 (br s, 1 H), 2.22 (br d, J = 11.49 Hz, 1 H), 1.92 (br t, J = 13.02, 13.02 Hz, 2 H), 1.71 (br d, J = 11.98 Hz, 1 H), 1.59 (m, 2 H), 1.30 (m, 2 H), 1.11 (m, 2 H). LC/MS: m/z [M + H]⁺ 451.3, Rt (I = 220 nm): 2.34 min (LC/MS method A), calc. m/z: 451.49. |
| 57 | 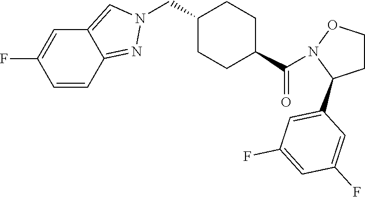 [SMILES: O=C([C@H]1CC[C@H](Cn2cc3cc(F)ccc3n2)CC1)N1OCC[C@H]1c1cc(F)cc(F)c1] | 1H NMR (400.23 MHz, DMSO-d6) d ppm 8.32 (s, 1 H), 7.66 (dd, J = 9.35, 4.71 Hz, 1 H), 7.42 (dd, J = 9.66, 2.45 Hz, 1 H), 7.12 (m, 2 H), 6.96 (br d, J = 6.48 Hz, 2 H), 5.31 (m, 1 H), 4.28 (d, J = 7.09 Hz, 2 H), 4.23 (td, J = 7.79, 7.79, 3.00 Hz, 1 H), 3.84 (m, 1 H), 2.85 (m, 1 H), 2.67 (br s, 1 H), 2.17 (m, 1 H), 1.92 (m, 2 H), 1.70 (br d, J = 11.49 Hz, 1 H), 1.58 (br t, J = 11.68, 11.68 Hz, 2 H), 1.30 (m, 2 H), 1.12 (m, 2 H). LC/MS: m/z [M + H]⁺ 444.3, Rt (I = 220 nm): 2.47 min (LC/MS method A), calc. m/z: 444.47. |
| 58 | 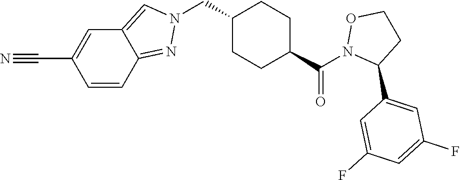 [SMILES: N#Cc1ccc2nn(C[C@H]3CC[C@H](C(=O)N4OCC[C@H]4c4cc(F)cc(F)c4)CC3)cc2c1] | 1H NMR (400.23 MHz, DMSO-d6) d ppm 8.63 (s, 1 H), 8.42 (s, 1 H), 7.77 (d, J = 8.93 Hz, 1 H), 7.46 (dd, J = 8.99, 1.53 Hz, 1 H), 7.11 (tt, J = 9.34, 9.34, 2.34, 2.34 Hz, 1 H), 6.96 (m, 2 H), 5.31 (dd, J = 8.44, 6.36 Hz, 1 H), 4.36 (d, J = 7.09 Hz, 2 H), 4.23 (td, J = 7.73, 7.73, 3.00 Hz, 1 H), 3.84 (m, 1 H), 3.58 (s, 1 H), 2.85 (m, 1 H), 2.67 (br d, J = 1.71 Hz, 1 H), 2.17 (m, 1 H), 1.97 (br dd, J = 7.21, 3.42 Hz, 1 H), 1.90 (br d, J = 12.23 Hz, 1 H), 1.70 (br d, J = 11.86 Hz, 1 H), 1.58 (br t, J = 10.51, 10.51 Hz, 2 H), 1.31 (m, 2 H), 1.13 (m, 2H). LC/MS: m/z [M + H]⁺ 451.2, Rt (I = 220 nm): 2.33 min (LC/MS method A), calc. m/z: 451.49. |
| 59 | 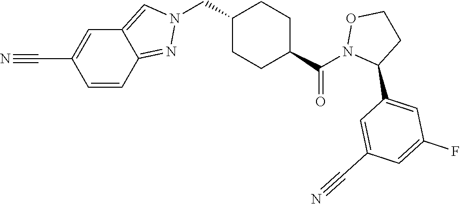 [SMILES: N#Cc1cc(F)cc([C@@H]2CCON2C(=O)[C@H]2CC[C@H](Cn3cc4cc(C#N)ccc4n3)CC2)c1] | 1H NMR (400.23 MHz, DMSO-d6) d ppm 8.63 (s, 1 H), 8.42 (s, 1 H), 7.77 (t, J = 8.27, 8.27 Hz, 2 H), 7.58 (s, 1 H), 7.47 (m, 1 H), 7.45 (s, 1 H), 5.35 (m, 1 H), 4.36 (d, J = 6.97 Hz, 2 H), 4.25 (td, J = 7.70, 7.70, 2.93 Hz, 1 H), 3.86 (m, 1 H), 2.87 (m, 1 H), 2.67 (br s, 1 H), 2.21 (m, 1 H), 1.97 (br d, J = 3.18 Hz, 1 H), 1.90 (br d, J = 13.33 Hz, 1 H), 1.71 (br d, J = 11.49 Hz, 1 H), 1.58 (br t, J = 8.86, 8.86 Hz, 2 H), 1.30 (m, 2 H), 1.13 (m, 2 H). LC/MS: m/z [M + H]⁺ 458.2, Rt (I = 220 nm): 2.2 min (LC/MS method A), calc. m/z: 458.51. |
| 60 | 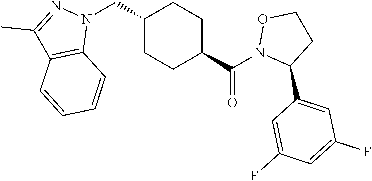 [SMILES: Cc1nn(C[C@H]2CC[C@H](C(=O)N3OCC[C@H]3c3cc(F)cc(F)c3)CC2)c2ccccc12] | ¹H NMR (400 MHz, DMSO-d₆): δ 7.68 (d, J = 7.95 Hz, 1 H), 7.57 (d, J = 8.44 Hz, 1 H), 7.34 (t, J = 7.58, 7.58 Hz, 1 H), 7.10 (m, 2 H), 6.95 (br d, J = 6.48 Hz, 2 H), 5.30 (m, 1 H), 4.22 (td, J = 7.79, 7.79, 2.87 Hz, 1 H), 4.17 (d, J = 6.97 Hz, 2 H), 3.83 (m, 1 H), 2.85 (m, 1 H), 2.65 (m, 1 H), 2.17 (m, 1 H), 1.87 (br d, J = 11.37 Hz, 2 H), 1.67 (br d, J = 11.86 Hz, 1 H), 1.57 (br t, J = 13.63 13.63 Hz, 2 H), 1.27 (m, 2 H), 1.13 (m, 2 H). LC/MS: m/z [M + H]⁺ 440.3, Rt (I = 220 nm): 2.65 min (LC/MS method A), calc. m/z: 440.51. |
| 61 | 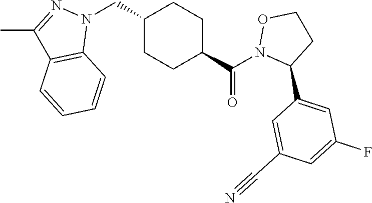 [SMILES: Cc1nn(C[C@H]2CC[C@H](C(=O)N3OCC[C@H]3c3cc(F)cc(C#N)c3)CC2)c2ccccc12] | 1H NMR (400.23 MHz, DMSO-d6) d ppm 7.75 (d, J = 8.65 Hz, 1 H), 7.68 (d, J = 8.07 Hz, 1 H), 7.58 (d, J = 1.47 Hz, 1 H), 7.56 (s, 1 H), 7.45 (br d, J = 9.66 Hz, 1 H), 7.34 (ddd, J = 8.28, 7.00, 0.98 Hz, 1 H), 7.08 (t, J = 7.21, 7.21 Hz, 1 H), 5.34 (dd, J = 8.50, 6.54 Hz, 1 H), 4.24 (td, J = 7.79, 7.79, 3.12 Hz, 1 H), 4.17 (d, J = 6.97 Hz, 2 H), 3.85 (m, 1 H), 2.86 (m, 1 H), 2.66 (m, 1 H), 2.20 (m, 1 H), 1.87 (br d, J = 11.00 Hz, 2 H), 1.68 (br d, J = 13.33 Hz, 1 H), 1.58 (br t, J = 12.72, 12.72 Hz, 2 H), 1.17 m (1H). LC/MS: m/z [M + H]⁺ 447.3, Rt (I = 220 nm): 2.52 min (LC/MS method A), calc. m/z: 444.52. |
| 62 | 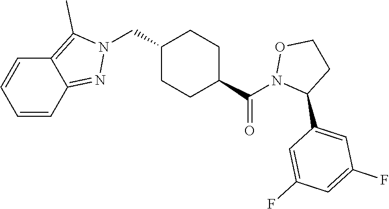 [SMILES: Cc1c2ccccc2nn1C[C@H]1CC[C@H](C(=O)N2OCC[C@H]2c2cc(F)cc(F)c2)CC1] | 1H NMR (400.23 MHz, DMSO-d6) d ppm 7.64 (d, J = 8.44 Hz, 1 H), 7.49 (d, J = 8.68 Hz, 1 H), 7.18 (t, J = 7.27, 7.27 Hz, 1 H), 7.11 (m, 1 H), 6.96 (br d, J = 8.31 Hz, 3 H), 5.31 (m, 1 H), 4.21 (m, 3 H), 3.84 (m, 1 H), 2.86 (br dd, J = 6.24, 2.81 Hz, 1 H), 2.67 (br s, 1 H), 2.61 (s, 3 H), 2.17 (br s, 1 H), 1.96 (br s, 1 H), 1.89 (br d, J = 13.94 Hz, 1 H), 1.68 (br s, 1 H), 1.59 (br t, J = 12.53, 12.53 Hz, 2 H), 1.29 (m, 2 H), 1.89 (br d, J = 12.10 Hz, 2 H). LC/MS: m/z [M + H]⁺ 440.3, Rt (I = 220 nm): 2.44 min (LC/MS method A), calc. m/z: 440.51. |
| 63 | 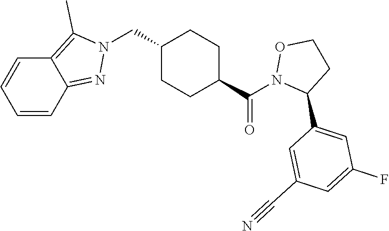 [SMILES: Cc1c2ccccc2nn1C[C@H]1CC[C@H](C(=O)N2OCC[C@H]2c2cc(F)cc(C#N)c2)CC1] | 1H NMR (400.23 MHz, DMSO-d6) d ppm 7.75 (br d, J = 7.34 Hz, 1 H), 7.64 (d, J = 8.50 Hz, 1 H), 7.58 (s, 1 H), 7.49 (d, J = 8.79 Hz, 1 H), 7.45 (d, J = 9.58 Hz, 1 H), 7.18 (t, J = 7.62, 7.62 Hz, 1 H), 6.95 (t, J = 7.44, 7.44 Hz, 1 H), 5.35 (m, 1 H), 4.25 (m, 1 H), 4.20 (d, J = 7.21 Hz, 2 H), 3.86 (m, 1 H), 3.16 (d, J = 2.69 Hz, 1 H), 2.87 (td, J = 6.02, 6.02, 2.51 Hz, 1 H), 2.68 (m, 1 H), 2.61 (s, 3 H), 2.21 (br dd, J = 13.14, 7.27 Hz, 1 H), 1.91 (m, 2 H), 1.71 (br d, J = 12.47 Hz, 1 H), 1.59 (br t, J = 13.08, 2H), 1.31-1.10 (m, 3H). LC/MS: m/z [M + H]⁺ 447.3, Rt (I = 220 nm): 2.31 min (LC/MS method A), calc. m/z: 447.52. |
| 64 | 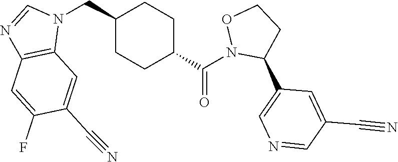 [SMILES: N#Cc1cncc([C@@H]2CCON2C(=O)[C@H]2CC[C@H](Cn3cnc4cc(F)c(C#N)cc43)CC2)c1] | ¹H NMR (400 MHz, DMSO-d₆): δ ppm 8.93 (d, J = 1.96 Hz, 1 H), 8.76 (d, J = 2.20 Hz, 1 H), 8.55 (s, 1 H), 8.42 (d, J = 5.75 Hz, 1 H), 8.17 (t, J = 1.96, 1.96 Hz, 1 H), 7.78 (d, J = 10.39 Hz, 1 H), 5.39 (dd, J = 8.56, 6.48 Hz, 1 H), 4.28 (td, J = 7.73, 7.73, 3.12 Hz, 1 H), 4.17 (d, J = 7.21 Hz, 2 H), 3.90 (m, 1 H), 2.89 (dddd, J = 12.12, 9.15, 6.48, 3.18 Hz, 1 H), 2.67 (m, 1 H), 2.28 (m, 1 H), 1.88 (br dd, J = 7.34, 3.55 Hz, 2 H), 1.72 (br d, J = 12.47 Hz, 1 H), 1.56 (br t, J = 8.93, 8.93 Hz, 2 H), 1.29 (m, 2 H), 1.10 (m, 2 H). LC/MS: m/z [M + H]⁺ 459.3, Rt (I = 220 nm): 1.81 min (LC/MS method A), calc. m/z: 459.50. |
| 65 | 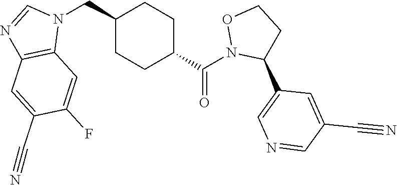 [SMILES: N#Cc1cncc([C@@H]2CCON2C(=O)[C@H]2CC[C@H](Cn3cnc4cc(C#N)c(F)cc43)CC2)c1] | 1H NMR (400.23 MHz, DMSO-d6) d ppm 8.93 (d, J = 1.96 Hz, 1 H), 8.76 (d, J = 2.20 Hz, 1 H), 8.47 (s, 1 H), 8.28 (d, J = 5.75 Hz, 1 H), 8.17 (t, J = 1.96, 1.96 Hz, 1 H), 7.95 (d, J = 9.90 Hz, 1 H), 5.39 (dd, J = 8.44, 6.60 Hz, 1 H), 4.28 (td, J = 7.76, 7.76, 3.06 Hz, 1 H), 4.15 (d, J = 7.09 Hz, 2 H), 3.90 (m, 1 H), 2.88 (dddd, J = 12.18, 9.15, 6.42, 3.06 Hz, 1 H), 2.67 (m, 1 H), 2.28 (m, 1 H), 1.86 (m, 2 H), 1.71 (br d, J = 11.62 Hz, 1 H), 1.57 (br t, J = 11.74, 11.74 Hz, 2 H), 1.29 (m, 2 H), 1.10 (m, 2 H). LC/MS: m/z [M + H]⁺ 459.3, Rt (I = 220 nm): 1.82 min (LC/MS method A), calc. m/z: 459.50. |
| 66 | 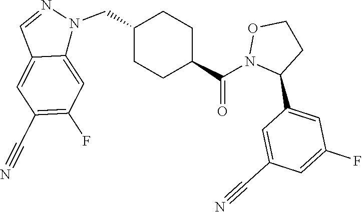 [SMILES: N#Cc1cc(F)cc([C@@H]2CCON2C(=O)[C@H]2CC[C@H](Cn3ncc4cc(C#N)c(F)cc43)CC2)c1] | ¹H NMR (400 MHz, DMSO-d₆): δ ppm 8.49 (d, J = 6.24 Hz, 1 H), 8.29 (s, 1 H), 7.97 (d, J = 10.51 Hz, 1 H), 7.75 (br d, J = 8.56 Hz, 1 H), 7.58 (s, 1 H), 7.45 (br d, J = 9.54 Hz, 1 H), 5.34 (m, 1 H), 4.29 (d, J = 6.97 Hz, 2 H), 4.24 (td, J = 7.70, 7.70, 2.93 Hz, 1 H), 3.85 (m, 1 H), 2.87 (m, 1 H), 2.67 (br d, J = 1.83 Hz, 1 H), 2.21 (m, 1 H), 1.88 (br d, J = 11.37 Hz, 2 H), 1.69 (br d, J = 11.37 Hz, 1 H), 1.55 (br t, J = 13.88, 13.88 Hz, 2 H), 1.27 (m, 2 H), 1.13 (m, 2 H). LC/MS: m/z [M + H]⁺ 476.3, Rt (I = 220 nm): 2.37 min (LC/MS method A), calc. m/z: 476.50. |
| 67 | 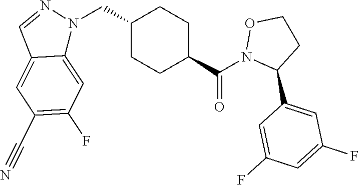 [SMILES: N#Cc1cc2cnn(C[C@H]3CC[C@H](C(=O)N4OCC[C@H]4c4cc(F)cc(F)c4)CC3)c2cc1F] | ¹H NMR (400 MHz, DMSO-d₆): δ ppm 8.49 (d, J = 6.24 Hz, 1 H), 8.29 (s, 1 H), 7.97 (d, J = 10.39 Hz, 1 H), 7.11 (tt, J = 9.32, 9.32, 2.29, 2.29 Hz, 1 H), 6.95 (br d, J = 6.60 Hz, 2 H), 5.30 (m, 1 H), 4.29 (d, J = 6.97 Hz, 2 H), 4.22 (td, J = 7.76, 7.76, 3.06 Hz, 1 H), 3.84 (m, 1 H), 2.85 (m, 1 H), 2.67 (br d, J = 2.08 Hz, 1 H), 2.65 (br s, 1 H), 2.17 (m, 1 H), 1.88 (br d, J = 11.13 Hz, 2 H), 1.68 (br d, J = 12.35 Hz, 1 H), 1.55 (br t, J = 14.06, 14.06 Hz, 2 H), 1.27 (m, 2 H), 1.13 (m, 2 H). LC/MS: m/z [M + H]⁺ 469.3, Rt (I = 220 nm): 2.49 min (LC/MS method A), calc. m/z: 469.48. |
| 68 | 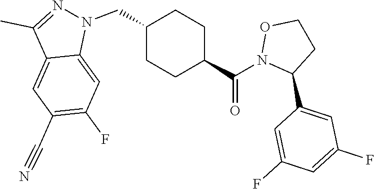 [SMILES: Cc1nn(C[C@H]2CC[C@H](C(=O)N3OCC[C@H]3c3cc(F)cc(F)c3)CC2)c2cc(F)c(C#N)cc12] | ¹H NMR (400 MHz, DMSO-d₆): δ ppm 8.49 (d, J = 6.24 Hz, 1 H), 7.86 (d, J = 10.64 Hz, 1 H), 7.11 (tt, J = 9.31, 9.31, 2.31, 2.31 Hz, 1 H), 6.95 (br d, J = 6.48 Hz, 2 H), 5.30 (m, 1 H), 4.30 (m, 3 H), 3.83 (m, 1 H), 2.85 (m, 1 H), 2.67 (br d, J = 1.59 Hz, 1 H), 2.17 (m, 1 H), 1.87 (br d, J = 8.07 Hz, 2 H), 1.68 (br d, J = 11.98 Hz, 1 H), 1.55 (m, 2 H), 1.27 (m, 2 H), 1.12 (m, 2 H). LC/MS: m/z [M + H]⁺ 483.3, Rt (I = 220 nm): 2.58 min (LC/MS method A), calc. m/z: 483.51. |
| 69 | 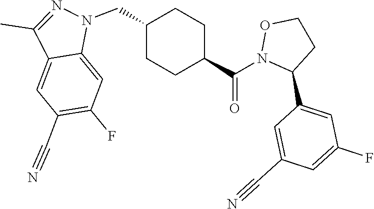 [SMILES: Cc1nn(C[C@H]2CC[C@H](C(=O)N3OCC[C@H]3c3cc(F)cc(C#N)c3)CC2)c2cc(F)c(C#N)cc12] | ¹H NMR (400 MHz, DMSO-d₆): δ ppm 8.49 (d, J = 6.24 Hz, 1 H), 7.86 (d, J = 10.51 Hz, 1 H), 7.75 (d, J = 8.65 Hz, 1 H), 7.58 (s, 1 H), 7.45 (br d, J = 9.54 Hz, 1 H), 5.35 (m, 1 H), 4.24 (td, J = 7.73, 7.73, 3.12 Hz, 1 H), 4.19 (d, J = 6.97 Hz, 2 H), 3.86 (m, 1 H), 2.87 (m, 1 H), 2.67 (br d, J = 1.83 Hz, 1 H), 2.63 (br s, 1 H), 2.21 (m, 1 H), 1.87 (br d, J = 11.74 Hz, 2 H), 1.69 (br d, J = 13.08 Hz, 1 H), 1.56 (br t, J = 13.20, 13.20 Hz, 2 H), 1.27 (m, 2 H), 1.13 (m, 2 H). LC/MS: m/z [M + H]⁺ 490.3, Rt (I = 220 nm): 2.46 min (LC/MS method A), calc. m/z: 490.52. |
| 70 | 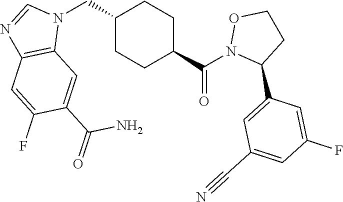 [SMILES: N#Cc1cc(F)cc([C@@H]2CCON2C(=O)[C@H]2CC[C@H](Cn3cnc4cc(F)c(C(N)=O)cc43)CC2)c1] | 1H NMR (400.23 MHz, DMSO-d6) d ppm 8.69 (s, 1 H), 8.06 (d, J = 6.11 Hz, 1 H), 7.72 (m, 3 H), 7.60 (d, J = 11.66 Hz, 1 H), 7.58 (s, 1 H), 7.45 (br d, J = 9.54 Hz, 1 H), 5.35 (m, 1 H), 4.23 (m, 3 H), 4.02 (m, 1 H), 3.86 (m, 4 H), 2.87 (m, 1 H), 2.67 (br d, J = 1.96 Hz, 1 H), 2.21 (m, 1 H), 1.90 (br d, J = 12.10 Hz, 2 H), 1.72 (br d, J = 12.47 Hz, 1 H), 1.59 (br t, J = 10.27, 10.27 Hz, 2 H), 1.29 (m, 2 H), 1.13 (m, 2 H). LC/MS: m/z [M + H]⁺ 494.3, Rt (I = 220 nm): 1.59 min (LC/MS method A), calc. m/z: 494.51. |
| 71 | 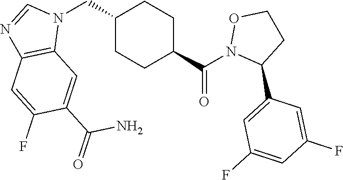 [SMILES: NC(=O)c1cc2c(cc1F)ncn2C[C@H]1CC[C@H](C(=O)N2OCC[C@H]2c2cc(F)cc(F)c2)CC1] | 1H NMR (400.23 MHz, DMSO-d6) d ppm 8.69 (s, 1 H), 8.06 (d, J = 6.11 Hz, 1 H), 7.69 (br d, J = 8.68 Hz, 2 H), 7.60 (d, J = 11.13 Hz, 1 H), 7.11 (tt, J = 9.35, 9.35, 2.32, 2.32 Hz, 1 H), 6.96 (br d, J = 6.60 Hz, 2 H), 5.31 (m, 1 H), 4.22 (m, 3 H), 4.03 (s, 1 H), 3.85 (m, 2 H), 3.81 (s, 2 H), 2.85 (m, 1 H), 2.67 (br d, J = 1.83 Hz, 1 H), 2.17 (m, 1 H), 1.89 (br d, J = 11.37 Hz, 2 H), 1.70 (br d, J = 12.96 Hz, 1 H), 1.60 (br t, J = 11.55, 11.55 Hz, 2 H), 1.31 (m, 2 H), 1.13 (m, 2 H). LC/MS: m/z [M + H]⁺ 487.3, Rt (I = 220 nm): 1.72 min (LC/MS method A), calc. m/z: 487.49. |
| 72 | 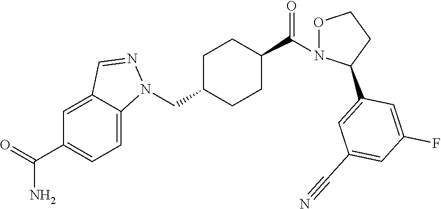 [SMILES: N#Cc1cc(F)cc([C@@H]2CCON2C(=O)[C@H]2CC[C@H](Cn3ncc4cc(C(N)=O)ccc43)CC2)c1] | 1H NMR (400.23 MHz, DMSO-d6) d ppm 8.34 (s, 1 H), 8.18 (s, 1 H), 7.90 (m, 2 H), 7.74 (m, 2 H), 7.58 (s, 1 H), 7.45 (br d, J = 9.66 Hz, 1 H), 7.26 (br s, 1 H), 5.34 (m, 1 H), 4.30 (d, J = 6.85 Hz, 2 H), 4.24 (td, J = 7.70, 7.70, 2.81 Hz, 1 H), 3.85 (m, 1 H), 2.86 (dddd, J = 12.04, 9.11, 6.48, 3.06 Hz, 1 H), 2.67 (br d, J = 1.83 Hz, 1 H), 2.20 (m, 1 H), 1.88 (br d, J = 11.86 Hz, 2 H), 1.69 (br d, J = 12.47 Hz, 1 H), 1.56 (br t, J = 14.18, 14.18 Hz, 2 H), 1.25 (m, 2 H), 1.13 (m, 2 H). LC/MS: m/z [M + H]⁺ 476.3, Rt (I = 220 nm): 1.86 min (LC/MS method A), calc. m/z: 476.52. |
| 73 | 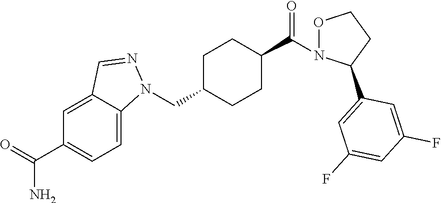 [SMILES: NC(=O)c1ccc2c(cnn2C[C@H]2CC[C@H](C(=O)N3OCC[C@H]3c3cc(F)cc(F)c3)CC2)c1] | 1H NMR (400.23 MHz, DMSO-d6) d ppm 8.34 (s, 1 H), 8.18 (s, 1 H), 7.96 (br s, 1 H), 7.90 (dd, J = 8.86, 1.53 Hz, 1 H), 7.72 (d, J = 8.93 Hz, 1 H), 7.25 (br s, 1 H), 7.11 (tt, J = 9.34, 9.34, 2.28, 2.28 Hz, 1 H), 6.95 (br d, J = 6.60 Hz, 2 H), 5.30 (dd, J = 8.50, 6.30 Hz, 1 H), 4.26 (m, 3 H), 3.83 (m, 1 H), 2.85 (dddd, J = 12.17, 9.14, 6.57, 3.06 Hz, 1 H), 2.67 (m, 1 H), 2.17 (m, 1 H), 1.87 (br d, J = 12.23 Hz, 2 H), 1.67 (br d, J = 10.64 Hz, 1 H), 1.56 (br t, J = 14.37, 14.37 Hz, 2 H), 1.26 (m, 2 H), 1.13 (m, 2 H). LC/MS: m/z [M + H]⁺ 469.3, Rt (I = 220 nm): 1.98 min (LC/MS method A), calc. m/z: 469.50. |
| 74 | 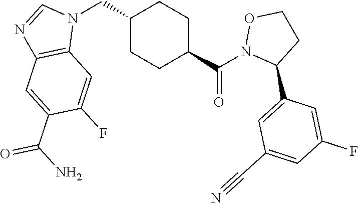 [SMILES: N#Cc1cc(F)cc([C@@H]2CCON2C(=O)[C@H]2CC[C@H](Cn3cnc4cc(C(N)=O)c(F)cc43)CC2)c1] | 1H NMR (400.23 MHz, DMSO-d6) d ppm 8.62 (s, 1 H), 7.96 (d, J = 6.36 Hz, 1 H), 7.76 (m, 2 H), 7.68 (br s, 1 H), 7.58 (s, 2 H), 7.46 (br d, J = 9.54 Hz, 1 H), 5.35 (m, 1 H), 4.20 (m, 3 H), 3.86 (m, 1 H), 2.87 (dddd, J = 12.10, 9.11, 6.36, 3.00 Hz, 1 H), 2.67 (br d, J = 1.71 Hz, 1 H), 2.21 (m, 1 H), 1.90 (br d, J = 12.59 Hz, 2 H), 1.71 (br d, J = 12.35 Hz, 1 H), 1.60 (br t, J = 12.35, 12.35 Hz, 2 H), 1.29 (m, 2 H), 1.13 (m, 2 H). LC/MS: m/z [M + H]⁺ 494.3, Rt (I = 220 nm): 1.66 min (LC/MS method A), calc. m/z: 494.51. |
| 75 | 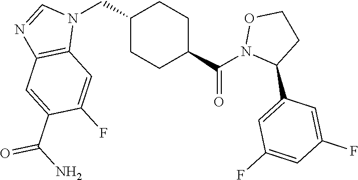 [SMILES: NC(=O)c1cc2ncn(C[C@H]3CC[C@H](C(=O)N4OCC[C@H]4c4cc(F)cc(F)c4)CC3)c2cc1F] | 1H NMR (400.23 MHz, DMSO-d6) d ppm 8.63 (s, 1 H), 7.96 (d, J = 6.36 Hz, 1 H), 7.77 (d, J = 10.76 Hz, 1 H), 7.69 (br s, 1 H), 7.58 (br s, 1 H), 7.12 (tt, J = 9.28, 9.28, 2.28, 2.28 Hz, 1 H), 6.96 (br d, J = 6.60 Hz, 2 H), 5.31 (m, 1 H), 4.23 (td, J = 7.64, 7.64, 3.06 Hz, 1 H), 4.17 (d, J = 7.09 Hz, 2 H), 3.84 (m, 1 H), 2.85 (m, 1 H), 2.67 (br s, 1 H), 2.17 (m, 1 H), 1.89 (br d, J = 12.47 Hz, 2 H), 1.70 (br d, J = 10.39 Hz, 1 H), 1.60 (br t, J = 12.59, 12.59 Hz, 2 H), 1.30 (m, 2 H), 1.13 (m, 2 H). LC/MS: m/z [M + H]⁺ 487.3, Rt (I = 220 nm): 1.8 min (LC/MS method A), calc. m/z: 487.49. |
| 76 | 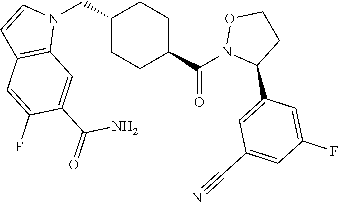 [SMILES: N#Cc1cc(F)cc([C@@H]2CCON2C(=O)[C@H]2CC[C@H](Cn3ccc4cc(F)c(C(N)=O)cc43)CC2)c1] | 1H NMR (400.23 MHz, DMSO-d6) d ppm 7.85 (d, J = 6.11 Hz, 1 H), 7.75 (d, J = 8.65 Hz, 1 H), 7.52 (m, 5 H), 7.36 (d, J = 11.98 Hz, 1 H), 6.46 (d, J = 2.81 Hz, 1 H), 5.35 (m, 1 H), 4.25 (td, J = 7.76, 7.76, 2.69 Hz, 1 H), 4.07 (d, J = 7.21 Hz, 2 H), 3.85 (m, 1 H), 2.87 (dddd, J = 12.15, 9.12, 6.48, 3.06 Hz, 1 H), 2.67 (br d, J = 1.83 Hz, 1 H), 2.21 (m, 1 H), 1.88 (br d, J = 12.23 Hz, 1 H), 1.81 (br s, 1 H), 1.70 (br d, J = 12.96 Hz, 1 H), 1.55 (br t, J = 13.33, 13.33 Hz, 2 H), 1.27 (m, 2 H), 1.11 (m, 2 H). LC/MS: m/z [M + H]⁺ 493.3, Rt (I = 220 nm): 2.21 min (LC/MS method A), calc. m/z: 493.53. |
| 77 | 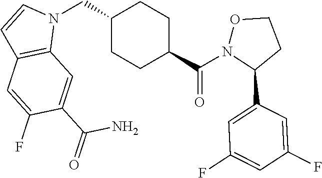 [SMILES: NC(=O)c1cc2c(ccn2C[C@H]2CC[C@H](C(=O)N3OCC[C@H]3c3cc(F)cc(F)c3)CC2)cc1F] | ¹H NMR (400 MHz, DMSO-d₆): δ ppm 7.84 (d, J = 5.99 Hz, 1 H), 7.53 (m, 3 H), 7.36 (d, J = 12.10 Hz, 1 H), 7.11 (tt, J = 9.32, 9.32, 2.35, 2.35 Hz, 1 H), 6.95 (br d, J = 6.60 Hz, 2 H), 6.46 (d, J = 2.81 Hz, 1 H), 5.31 (m, 1 H), 4.23 (td, J = 7.76, 7.76, 2.81 Hz, 1 H), 4.07 (d, J = 7.21 Hz, 2 H), 3.83 (m, 1 H), 2.85 (m, 1 H), 2.67 (br s, 1 H), 2.17 (m, 1 H), 1.85 (m, 2 H), 1.69 (br d, J = 11.86 Hz, 1 H), 1.55 (m, 2 H), 1.27 (m, 2 H), 1.11 (m, 2 H). LC/MS: m/z [M + H]⁺ 486.3, Rt (I = 220 nm): 2.33 min (LC/MS method A), calc. m/z: 486.51. |
| 78 | 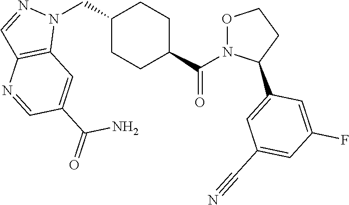 [SMILES: N#Cc1cc(F)cc([C@@H]2CCON2C(=O)[C@H]2CC[C@H](Cn3ncc4ncc(C(N)=O)cc43)CC2)c1] | 1H NMR (400.23 MHz, DMSO-d6) d ppm 8.98 (d, J = 1.71 Hz, 1 H), 8.65 (s, 1 H), 8.36 (d, J = 0.73 Hz, 1 H), 8.22 (br s, 1 H), 7.75 (d, J = 8.67 Hz, 1 H), 7.68 (br s, 1 H), 7.58 (s, 1 H), 7.45 (br d, J = 9.54 Hz, 1 H), 5.35 (dd, J = 8.56, 6.48 Hz, 1 H), 4.35 (d, J = 7.09 Hz, 2 H), 4.24 (td, J = 7.76, 7.76, 3.06 Hz, 1 H), 3.85 (m, 3 H), 2.86 (dddd, J = 12.15, 9.15, 6.45, 3.06 Hz, 1 H), 2.67 (m, 1 H), 2.20 (m, 1 H), 1.91 (m, 2 H), 1.70 (br d, J = 12.35 Hz, 1 H), 1.55 (br t, J = 14.49, 14.49 Hz, 2 H), 1.31-1.07 (m, 4 H). LC/MS: m/z [M + H]⁺ 477.3, Rt (I = 220 nm): 1.73 min (LC/MS method A), calc. m/z: 477.51. |
| 79 | 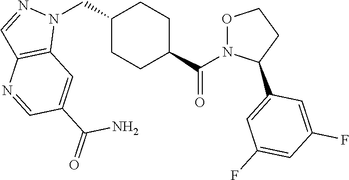 [SMILES: NC(=O)c1cnc2cnn(C[C@H]3CC[C@H](C(=O)N4OCC[C@H]4c4cc(F)cc(F)c4)CC3)c2c1] | 1H NMR (400.23 MHz, DMSO-d6) d ppm 8.98 (d, J = 1.71 Hz, 1 H), 8.64 (s, 1 H), 8.36 (s, 1 H), 8.22 (br s, 1 H), 7.68 (br s, 1 H), 7.11 (tt, J = 9.29, 9.29, 2.32, 2.32 Hz, 1 H), 6.95 (br d, J = 6.60 Hz, 2 H), 5.31 (m, 1 H), 4.35 (d, J = 7.09 Hz, 2 H), 4.22 (td, J = 7.64, 7.64, 2.81 Hz, 1 H), 3.83 (m, 1 H), 2.85 (dddd, J = 12.04, 9.08, 6.39, 3.06 Hz, 1 H), 2.67 (br s, 1 H), 2.17 (m, 1 H), 1.94 (br s, 1 H), 1.88 (br d, J = 11.98 Hz, 1 H), 1.68 (br d, J = 10.27 Hz, 1 H), 1.55 (br t, J = 14.98, 14.98 Hz, 2 H), 1.28 (m, 2 H). LC/MS: m/z [M + H]⁺ 470.3, Rt (I = 220 nm): 1.86 min (LC/MS method A), calc. m/z: 470.49. |
| 80 | 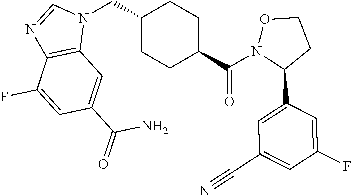 [SMILES: N#Cc1cc(F)cc([C@@H]2CCON2C(=O)[C@H]2CC[C@H](Cn3cnc4c(F)cc(C(N)=O)cc43)CC2)c1] | 1H NMR (400.23 MHz, DMSO-d6) d ppm 8.41 (s, 1 H), 8.06 (m, 2 H), 7.75 (d, J = 7.76 Hz, 1 H), 7.57 (m, 2 H), 7.46 (m, 2 H), 5.35 (m, 1 H), 4.21 (m, 3 H), 3.86 (m, 1 H), 2.87 (dddd, J = 12.10, 9.08, 6.33, 3.06 Hz, 1 H), 2.68 (br d, J = 8.07 Hz, 1 H), 2.21 (m, 1 H), 1.91 (br d, J = 11.37 Hz, 2 H), 1.72 (br d, J = 11.86 Hz, 1 H), 1.59 (m, 2 H), 1.30 (m, 2 H), 1.13 (m, 2 H). LC/MS: m/z [M + H]⁺ 494.3, Rt (I = 220 nm): 1.71 min (LC/MS method A), calc. m/z: 494.51. |
| 81 | 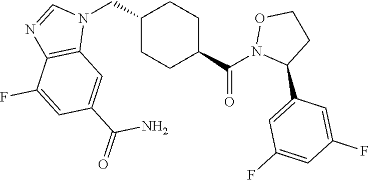 [SMILES: NC(=O)c1cc(F)c2ncn(C[C@H]3CC[C@H](C(=O)N4OCC[C@H]4c4cc(F)cc(F)c4)CC3)c2c1] | 1H NMR (400.23 MHz, DMSO-d6) d ppm 8.41 (s, 1 H), 8.06 (m, 2 H), 7.56 (d, J = 11.98 Hz, 1 H), 7.47 (br s, 1 H), 7.11 (m, 1 H), 6.96 (br d, J = 6.60 Hz, 2 H), 5.31 (m, 1 H), 4.20 (m, 3 H), 3.84 (m, 1 H), 2.85 (m, 1 H), 2.67 (br d, J = 1.96 Hz, 1 H), 2.17 (m, 1 H), 1.90 (br d, J = 11.13 Hz, 2 H), 1.69 (br s, 1 H), 1.59 (br t, J = 13.39, 13.39 Hz, 2 H), 1.30 (m, 2 H), 1.13 (m, 2 H). LC/MS: m/z [M + H]⁺ 487.3, Rt (I = 220 nm): 1.83 min (LC/MS method A), calc. m/z: 487.49. |
| 82 | 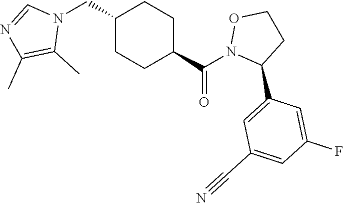 [SMILES: Cc1ncn(C[C@H]2CC[C@H](C(=O)N3OCC[C@H]3c3cc(F)cc(C#N)c3)CC2)c1C] | 1H NMR (400.23 MHz, DMSO-d6) d ppm 14.07 (br s, 1 H), 8.91 (s, 1 H), 7.77 (d, J = 8.62 Hz, 1 H), 7.59 (s, 1 H), 7.47 (br d, J = 9.54 Hz, 1 H), 5.36 (m, 1 H), 4.26 (td, J = 7.67, 7.67, 2.87 Hz, 1 H), 3.96 (d, J = 7.34 Hz, 2 H), 3.87 (m, 1 H), 3.43 (u), 2.89 (dddd, J = 12.17, 9.14, 6.39, 3.00 Hz, 1 H), 2.68 (m, 1 H), 2.22 (s, 6 H), 2.20 (m, 1 H), 1.92 (br d, J = 12.47 Hz, 1 H), 1.74 (br d, J = 11.49 Hz, 2 H), 1.61 (br d, J = 12.47 Hz, 2 H), 1.33 (m, 2 H), 1.09 (qd, J = 12.55, 12.55, 12.55, 12.35 Hz, 2 H). LC/MS: m/z [M + H]⁺ 411.3, Rt (I = 220 nm): 1.35 min (LC/MS method A), calc. m/z: 411.49. |
| 83 | 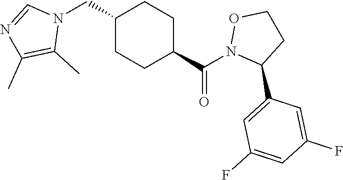 [SMILES: Cc1ncn(C[C@H]2CC[C@H](C(=O)N3OCC[C@H]3c3cc(F)cc(F)c3)CC2)c1C] | 1H NMR (400.23 MHz, DMSO-d6) d ppm 14.06 (br s, 1 H), 8.91 (s, 1 H), 7.13 (t, J = 9.27, 9.27 Hz, 1 H), 6.97 (br d, J = 6.60 Hz, 2 H), 5.32 (m, 1 H), 4.24 (m, 1 H), 3.96 (d, J = 7.34 Hz, 2 H), 3.85 (m, 1 H), 2.87 (m, 1 H), 2.68 (br d, J = 9.17 Hz, 1 H), 2.22 (s, 6 H), 2.16 (s, 1 H), 1.92 (br d, J = 13.20 Hz, 1 H), 1.74 (br s, 2 H), 1.60 (br d, J = 13.08 Hz, 2 H), 1.34 (m, 2 H), 1.09 (m, 2 H). LC/MS: m/z [M + H]⁺ 404.3, Rt (I = 220 nm): 1.46 min (LC/MS method A), calc. m/z: 404.47. |
| 84 | 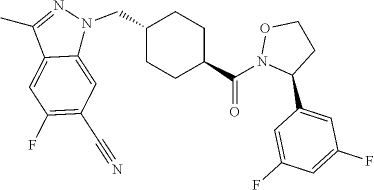 [SMILES: Cc1nn(C[C@H]2CC[C@H](C(=O)N3OCC[C@H]3c3cc(F)cc(F)c3)CC2)c2cc(C#N)c(F)cc12] | ¹H NMR (600 MHz, DMSO-d₆): δ ppm 8.48 (d, J = 4.95 Hz, 1 H), 7.88 (d, J = 9.54 Hz, 1 H), 7.11 (tt, J = 9.33, 9.33, 2.32, 2.32 Hz, 1 H), 6.96 (br d, J = 6.24 Hz, 2 H), 5.31 (m, 1 H), 4.24 (m, 3 H), 3.83 (m, 1 H), 2.85 (m, 1 H), 2.66 (br s, 1 H), 2.17 (m, 1 H), 1.88 (br d, J = 3.85 Hz, 1 H), 1.86 (br s, 1 H), 1.68 (br d, J = 10.09 Hz, 1 H), 1.54 (br dd, J = 18.71, 15.22 Hz, 2 H), 1.27 (m, 2 H), 1.13 (m, 2 H). LC/MS: m/z [M + H]⁺ 483.3, Rt (I = 220 nm): 2.65 min (LC/MS method A), calc. m/z: 483.51. |
| 85 | 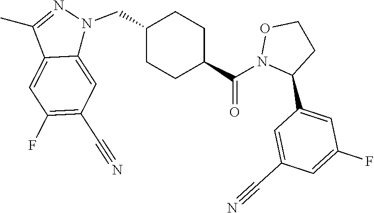 [SMILES: Cc1nn(C[C@H]2CC[C@H](C(=O)N3OCC[C@H]3c3cc(F)cc(C#N)c3)CC2)c2cc(C#N)c(F)cc12] | 1H NMR (600.05 MHz, DMSO-d6) d ppm 8.48 (d, J = 5.14 Hz, 1 H), 7.88 (d, J = 9.35 Hz, 1 H), 7.75 (d, J = 8.37 Hz, 1 H), 7.58 (s, 1 H), 7.45 (br d, J = 9.54 Hz, 1 H), 5.35 (m, 1 H), 4.25 (m, 3 H), 3.85 (m, 1 H), 2.87 (dddd, J = 12.17, 9.15, 6.46, 3.03 Hz, 1 H) 2.66 (br s, 1 H), 2.21 (m, 1 H), 1.87 (m, 2 H), 1.69 (br d, J = 12.10 Hz, 1 H), 1.54 (br dd, J = 17.97, 15.04 Hz, 2 H), 1.27 (m, 2 H), 1.13 (m, 2 H). LC/MS: m/z [M + H]⁺ 490.3, Rt (I = 220 nm): 2.53 min (LC/MS method A), calc. m/z: 490.52. |
| 86 | 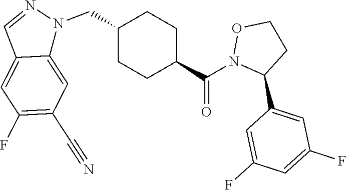 [SMILES: N#Cc1cc2c(cnn2C[C@H]2CC[C@H](C(=O)N3OCC[C@H]3c3cc(F)cc(F)c3)CC2)cc1F] | ¹H NMR (600 MHz, DMSO-d₆): δ ppm 8.59 (d, J = 5.14 Hz, 1 H), 8.23 (d, J = 0.92 Hz, 1 H), 7.88 (d, J = 9.35 Hz, 1 H), 7.11 (tt, J = 9.28, 9.28, 2.36, 2.36 Hz, 1 H), 6.95 (br d, J = 6.42 Hz, 2 H), 5.31 (m, 1 H), 4.35 (d, J = 7.15 Hz, 2 H), 4.23 (td, J = 7.79, 7.79, 2.93 Hz, 1 H), 3.83 (m, 1 H), 2.85 (m, 1 H), 2.66 (br s, 1 H), 2.17 (m, 1 H), 1.90 (m, 2 H), 1.67 (br s, 1 H), 1.54 (m, 2 H), 1.28 (m, 2 H), 1.14 (m, 2 H). LC/MS: m/z [M + H]⁺ 469.3, Rt (I = 220 nm): 2.55 min (LC/MS method A), calc. m/z: 469.48. |
| 87 | 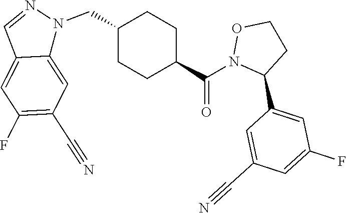 [SMILES: N#Cc1cc(F)cc([C@@H]2CCON2C(=O)[C@H]2CC[C@H](Cn3ncc4cc(F)c(C#N)cc43)CC2)c1] | ¹H NMR (400 MHz, DMSO-d₆): δ ppm 8.59 (d, J = 5.13 Hz, 1 H), 8.23 (d, J = 0.92 Hz, 1 H), 7.88 (d, J = 9.54 Hz, 1 H), 7.75 (d, J = 8.35 Hz, 1 H), 7.58 (s, 1 H), 7.45 (br d, J = 9.54 Hz, 1 H), 5.35 (m, 1 H), 4.35 (d, J = 6.97 Hz, 2 H), 4.24 (td, J = 7.79, 7.79, 2.93 Hz, 1 H), 3.85 (m, 1 H), 2.87 (m, 1 H), 2.67 (br s, 1 H), 2.21 (m, 1 H), 1.90 (m, 2 H), 1.69 (br d, J = 12.47 Hz, 1 H), 1.54 (m, 2 H), 1.27 (m, 2 H), 1.14 (m, 2 H). LC/MS: m/z [M + H]⁺ 476.3, Rt (I = 220 nm): 2.42 min (LC/MS method A), calc. m/z: 476.50. |
| 88 | 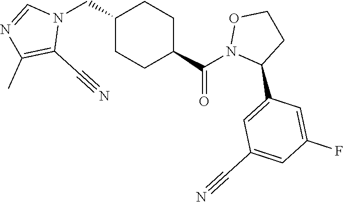 [SMILES: Cc1ncn(C[C@H]2CC[C@H](C(=O)N3OCC[C@H]3c3cc(F)cc(C#N)c3)CC2)c1C#N] | 1H NMR (400.23 MHz, DMSO-d6) d ppm 7.97 (s, 1 H), 7.76 (d, J = 7.90 Hz, 1 H), 7.59 (s, 1 H), 7.47 (br d, J = 9.54 Hz, 1 H), 5.36 (dd, J = 8.56, 6.48 Hz, 1 H), 4.25 (td, J = 7.73, 7.73, 2.87 Hz, 1 H), 3.90 (m, 7 H), 3.72 (br s, 33 H), 2.88 (dddd, J = 12.10, 9.14, 6.51, 3.06 Hz, 1 H), 2.68 (m, 1 H), 2.27 (s, 3 H), 2.21 (m, 1 H), 1.92 (br d, J = 12.84 Hz, 1 H), 1.74 (br dd, J = 7.46, 3.67 Hz, 2 H), 1.59 (m, 2 H), 1.32 (m, 2 H), 1.07 (m, 2 H). LC/MS: m/z [M + H]⁺ 422.2, Rt (I = 220 nm): 1.95 min (LC/MS method A), calc. m/z: 422.48. |
| 89 | 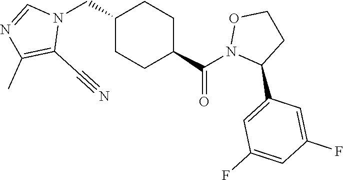 [SMILES: Cc1ncn(C[C@H]2CC[C@H](C(=O)N3OCC[C@H]3c3cc(F)cc(F)c3)CC2)c1C#N] | 1H NMR (400.23 MHz, DMSO-d6) d ppm 7.98 (s, 1 H), 7.12 (tt, J = 9.29, 9.29, 2.32, 2.32 Hz, 1 H), 6.97 (m, 2 H), 5.32 (dd, J = 8.44, 6.48 Hz, 1 H), 4.24 (td, J = 7.73, 7.73, 3.00 Hz, 2 H), 3.87 (m, 24 H), 2.86 (dddd, J = 12.12, 9.15, 6.48, 3.18 Hz, 1 H), 2.67 (br d, J = 1.71 Hz, 1 H), 2.27 (s, 3 H), 2.18 (m, 1 H), 1.92 (br d, J = 12.96 Hz, 1 H), 1.73 (br dd, J = 7.40, 3.61 Hz, 2 H), 1.61 (br d, J = 7.70 Hz, 2 H), 1.31 (dtt, J = 12.90, 12.70, 12.70, 3.47, 3.47 Hz, 2 H), 1.07 (m, 2 H). LC/MS: m/z [M + H]⁺ 415.3, Rt (I = 220 nm): 2.08 min (LC/MS method A), calc. m/z: 415.46. |
| 90 | 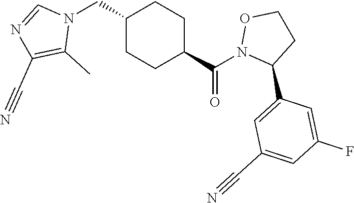 [SMILES: Cc1c(C#N)ncn1C[C@H]1CC[C@H](C(=O)N2OCC[C@H]2c2cc(F)cc(C#N)c2)CC1] | 1H NMR (400.23 MHz, DMSO-d6) d ppm 7.76 (s, 1 H), 7.76 (m, 1 H), 7.59 (s, 1 H), 7.47 (d, J = 9.47 Hz, 1 H), 5.36 (dd, J = 8.56, 6.48 Hz, 1 H), 4.25 (td, J = 7.70, 7.70, 3.06 Hz, 1 H), 3.85 (m, 3 H), 2.88 (dddd, J = 12.13, 9.14, 6.48, 3.06 Hz, 1 H), 2.68 (m, 1 H), 2.33 (s, 3 H), 2.22 (m, 1 H), 1.91 (br d, J = 12.23 Hz, 1 H), 1.70 (m, 2 H), 1.56 (br t, J = 8.50, 8.50 Hz, 2 H), 1.31 (m, 2 H), 1.07 (m, 2 H). LC/MS: m/z [M + H]⁺ 422.2, Rt (I = 220 nm): 1.94 min (LC/MS method A), calc. m/z: 422.48. |
| 91 | 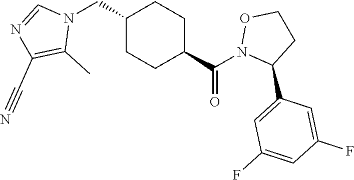 [SMILES: Cc1c(C#N)ncn1C[C@H]1CC[C@H](C(=O)N2OCC[C@H]2c2cc(F)cc(F)c2)CC1] | 1H NMR (400.23 MHz, DMSO-d6) d ppm 7.76 (s, 1 H), 7.12 (tt, J = 9.29, 9.29, 2.32, 2.32 Hz, 1 H), 6.97 (m, 2 H), 5.32 (dd, J = 8.68, 6.36 Hz, 1 H), 4.24 (td, J = 7.76, 7.76, 3.06 Hz, 1 H), 3.86 (m, 1 H), 3.83 (d, J = 7.09 Hz, 2 H), 3.58 (br s, 23 H), 2.86 (dddd, J = 12.09, 9.12, 6.48, 3.12 Hz, 1 H), 2.67 (br d, J = 1.83 Hz, 1 H), 2.33 (s, 3 H), 2.18 (m, 1 H), 1.90 (br d, J = 12.47 Hz, 1 H), 1.70 (m, 2 H), 1.56 (br t, J = 8.68, 8.68 Hz, 2 H), 1.32 (qdd, J = 12.72, 12.72, 12.72, 3.55, 3.30 Hz, 2 H), 1.07 (m, 2 H). LC/MS: m/z [M + H]⁺ 415.2, Rt (I = 220 nm): 2.08 min (LC/MS method A), calc. m/z: 415.46. |
| 92 | 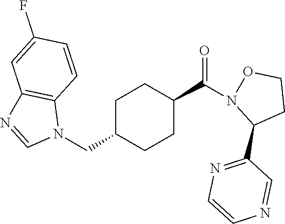 [SMILES: O=C([C@H]1CC[C@H](Cn2cnc3cc(F)ccc32)CC1)N1OCC[C@H]1c1cnccn1] | 1H NMR (400.23 MHz, DMSO-d6) d ppm 8.94 (s, 1 H), 8.58 (m, 3 H), 7.90 (dd, J = 9.05, 4.52 Hz, 1 H), 7.61 (dd, J = 9.11, 2.38 Hz, 1 H), 7.34 (td, J = 9.29, 9.29, 2.32 Hz, 1 H), 5.39 (dd, J = 8.68, 6.24 Hz, 1 H), 4.30 (td, J = 7.64, 7.64, 3.55 Hz, 2 H), 4.24 (br d, J = 7.09 Hz, 3 H), 3.93 (td, J = 8.41, 8.41, 6.66 Hz, 9 H), 3.51 (br s, 1 H), 2.81 (m, 1 H), 2.67 (br t, J = 11.43, 11.43 Hz, 1 H), 1.85 (m, 2 H), 1.73 (br d, J = 11.37 Hz, 1 H), 1.62 (br d, J = 11.62 Hz, 2 H), 1.28 (m, 2 H), 1.12 (m, 2 H). LC/MS: m/z [M + H]⁺ 410.2, Rt (I = 220 nm): 1.28 min (LC/MS method A), calc. m/z: 410.46. |
| 93 | 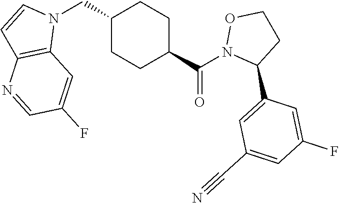 [SMILES: N#Cc1cc(F)cc([C@@H]2CCON2C(=O)[C@H]2CC[C@H](Cn3ccc4ncc(F)cc43)CC2)c1] | ¹H NMR (400 MHz, DMSO-d₆): δ ppm 8.43 (s, 1 H), 8.15 (br d, J = 9.90 Hz, 1 H), 7.76 (s, 1 H), 7.76 (d, J = 11.79 Hz, 1 H), 7.58 (s, 1 H), 7.45 (br d, J = 9.66 Hz, 1 H), 6.63 (d, J = 3.18 Hz, 1 H), 5.35 (m, 1 H), 4.24 (td, J = 7.70, 7.70, 2.93 Hz, 1 H), 4.07 (d, J = 7.09 Hz, 2 H), 3.86 (m, 1 H), 2.87 (m, 1 H), 2.67 (m, 1 H), 2.21 (m, 1 H), 1.84 (m, 2 H), 1.70 (br d, J = 12.72 Hz, 1 H), 1.55 (br t, J = 13.27, 13.27 Hz, 2 H), 1.27 (m, 2 H), 1.11 (m, 2 H). LC/MS: m/z [M + H]⁺ 451.3, Rt (I = 220 nm): 2.01 min (LC/MS method A), calc. m/z: 451.49. |
| 94 | 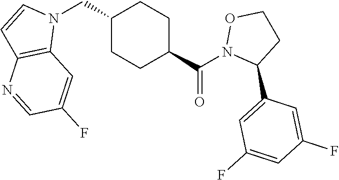 [SMILES: O=C([C@H]1CC[C@H](Cn2ccc3ncc(F)cc32)CC1)N1OCC[C@H]1c1cc(F)cc(F)c1] | ¹H NMR (400 MHz, DMSO-d₆): δ ppm 8.44 (t, J = 2.02, 2.02 Hz, 1 H), 8.16 (d, J = 9.72 Hz, 1 H), 7.77 (d, J = 3.18 Hz, 1 H), 7.11 (tt, J = 9.29, 9.29, 2.32, 2.32 Hz, 1 H), 6.95 (m, 2 H), 6.64 (d, J = 2.93 Hz, 1 H), 5.31 (dd, J = 8.38, 6.42 Hz, 1 H), 4.22 (td, J = 7.70, 7.70, 3.06 Hz, 1 H), 4.08 (d, J = 7.09 Hz, 2 H), 2.85 (m, 1 H), 2.67 (m, 1 H), 2.17 (m, 1 H), 1.84 (m, 2 H), 1.69 (br d, J = 12.59 Hz, 1 H), 1.55 (m, 2 H), 1.28 (m, 2 H), 1.11 (m, 2 H). LC/MS: m/z [M + H]⁺ 444.2, Rt (I = 220 nm): 2.15 min (LC/MS method A), calc. m/z: 444.47. |
| 95 | 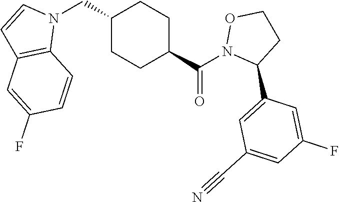 [SMILES: N#Cc1cc(F)cc([C@@H]2CCON2C(=O)[C@H]2CC[C@H](Cn3ccc4cc(F)ccc43)CC2)c1] | ¹H NMR (400 MHz, DMSO-d₆): δ ppm 7.75 (br dd, J = 8.50, 1.16 Hz, 1 H), 7.58 (s, 1 H), 7.45 (m, 3 H), 7.29 (dd, J = 9.90, 2.57 Hz, 1 H), 6.96 (m, 1 H), 6.41 (d, J = 2.81 Hz, 1 H), 5.35 (br t, J = 7.40, 7.40 Hz, 1 H), 4.24 (m, 1 H), 4.03 (br d, J = 6.97 Hz, 2 H), 3.85 (m, 1 H), 2.87 (m, 1 H), 2.67 (br s, 1 H), 2.21 (m, 1 H), 1.88 (br d, J = 12.84 Hz, 1 H), 1.79 (br s, 1 H), 1.69 (br d, J = 11.00 Hz, 1 H), 1.57 (br t, J = 13.20, 13.20 Hz, 2 H), 1.26 (m, 2 H), 1.10 (m, 2 H). LC/MS: m/z [M + H]⁺ 450.2, Rt (I = 220 nm): 2.71 min (LC/MS method A), calc. m/z: 450.50. |
| 96 | 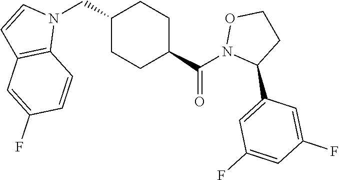 [SMILES: O=C([C@H]1CC[C@H](Cn2ccc3cc(F)ccc32)CC1)N1OCC[C@H]1c1cc(F)cc(F)c1] | ¹H NMR (400 MHz, DMSO-d₆): δ ppm 7.49 (dd, J = 8.93, 4.52 Hz, 1 H), 7.40 (d, J = 3.06 Hz, 1 H), 7.29 (dd, J = 9.90, 2.57 Hz, 1 H), 7.11 (tt, J = 9.35, 9.35, 2.32, 2.32 Hz, 1 H), 6.95 (m, 3 H), 6.41 (d, J = 2.81 Hz, 1 H), 5.30 (dd, J = 8.44, 6.48 Hz, 1 H), 4.22 (td, J = 7.76, 7.76, 2.93 Hz, 1 H), 4.03 (d, J = 7.09 Hz, 2 H), 3.83 (m, 1 H), 2.85 (m, 1 H), 2.67 (m, 1 H), 2.17 (m, 1 H), 1.87 (br d, J = 12.96 Hz, 1 H), 1.79 (m, 1 H), 1.70 (br d, J = 12.72 Hz, 1 H), 1.55 (m, 2 H), 1.27 (m, 2 H), 1.11 (m, 2 H). LC/MS: m/z [M + H]⁺ 443.2, Rt (I = 220 nm): 2.83 min (LC/MS method A), calc. m/z: 443.48. |
| 97 | 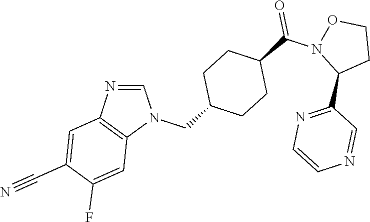 [SMILES: N#Cc1cc2ncn(C[C@H]3CC[C@H](C(=O)N4OCC[C@H]4c4cnccn4)CC3)c2cc1F] | 1H NMR (400.23 MHz, DMSO-d6) d ppm 8.58 (m, 3 H), 8.48 (s, 1 H), 8.28 (d, J = 5.75 Hz, 1 H), 7.95 (d, J = 9.90 Hz, 1 H), 5.39 (dd, J = 8.86, 6.17 Hz, 1 H), 4.30 (m, 1 H), 4.14 (d, J = 7.09 Hz, 2 H), 3.93 (m, 1 H), 2.81 (m, 1 H), 2.66 (m, 2 H), 1.83 (br d, J = 11.13 Hz, 2 H), 1.72 (br d, J = 11.37 Hz, 1 H), 1.57 (br d, J = 12.47 Hz, 2 H), 1.27 (m, 2 H), 1.10 (m, 2 H). LC/MS: m/z [M + H]⁺ 435.2, Rt (I = 220 nm): 1.68 min (LC/MS method A), calc. m/z: 435.48. |
| 98 | 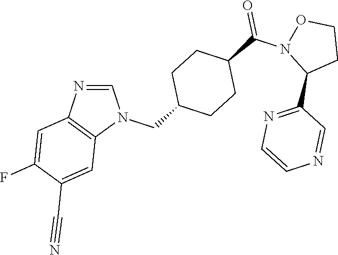 [SMILES: N#Cc1cc2c(cc1F)ncn2C[C@H]1CC[C@H](C(=O)N2OCC[C@H]2c2cnccn2)CC1] | 1H NMR (400.23 MHz, DMSO-d6) d ppm 8.58 (m, 4 H), 8.43 (d, J = 5.62 Hz, 1 H), 7.79 (d, J = 10.27 Hz, 1 H), 5.39 (dd, J = 8.80, 6.24 Hz, 1 H), 4.30 (m, 1 H), 4.17 (d, J = 7.21 Hz, 2 H), 3.93 (m, 3 H), 3.69 (br s, 41 H), 2.81 (m, 1 H), 2.66 (m, 1 H), 1.85 (m, 2 H), 1.72 (br d, J = 12.23 Hz, 1 H), 1.56 (br d, J = 11.98 Hz, 2 H), 1.28 (m, 2 H), 1.11 (m, 2 H). LC/MS: m/z [M + H]⁺ 435.2, Rt (I = 220 nm): 1.67 min (LC/MS method A), calc. m/z: 435.47. |
| 99 | 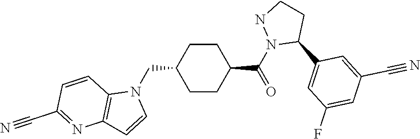 [SMILES: N#Cc1cc(F)cc([C@@H]2CCNN2C(=O)[C@H]2CC[C@H](Cn3ccc4nc(C#N)ccc43)CC2)c1] | ¹H NMR (400 MHz, CDCl₃): δ ppm 7.72 (d, J = 8.7 Hz, 1 H), 7.57-7.53 (m, 1 H), 7.49 (d, J = 3.3 Hz, 1 H), 7.39 (s, 1 H), 7.27-7.23 (m, 2 H), 6.83 (d, J = 2.9 Hz, 1 H), 5.44-5.33 (m, 1 H), 4.28 (dt, J = 2.3, 7.8 Hz, 1 H), 4.05 (br t, J = 6.4 Hz, 2 H), 3.93-3.80 (m, 1 H), 2.96-2.83 (m, 1 H), 2.83-2.70 (m, 1 H), 2.37-2.22 (m, 1 H), 2.09-1.99 (m, 1 H), 1.98-1.86 (m, 1 H), 1.85-1.76 (m, 2 H), 1.74- 1.68 (m, 1 H), 1.52-1.43 (m, 2 H), 1.22-1.06 (m, 2 H). LC/MS: m/z [M + H]⁺ 458.2, Rt (I = 220 nm): 0.713 min (LC/MS method A), calc. m/z: 458.20. |
| 100 | 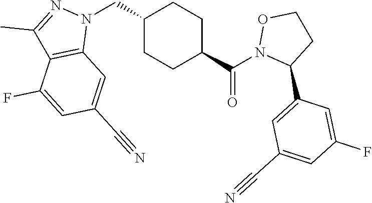 [SMILES: Cc1nn(C[C@H]2CC[C@H](C(=O)N3OCC[C@H]3c3cc(F)cc(C#N)c3)CC2)c2cc(C#N)cc(F)c12] | ¹H NMR (400 MHz, DMSO-d₆): δ ppm 8.27 (s, 1 H), 7.76 (m, 1 H), 7.58 (s, 1 H), 7.45 (br d, J = 9.66 Hz, 1 H), 7.32 (d, J = 10.27 Hz, 1 H), 5.35 (m, 1 H), 4.26 (m, 3 H), 3.85 (m, 1 H), 2.87 (m, 1 H), 2.67 (br d, J = 1.71 Hz, 1 H), 2.59 (s, 3 H), 2.21 (m, 1 H), 1.89 (m, 2 H), 1.69 (br d, J = 11.49 Hz, 1 H), 1.55 (br t, J = 13.33, 13.33 Hz, 2 H), 1.28 (m, 2 H), 1.14 (m, 2 H). LC/MS: m/z [M + H]⁺ 490.3, Rt (I = 220 nm): 2.62 min (LC/MS method A), calc. m/z: 490.52. |
| 101 | 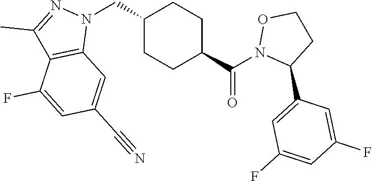 [SMILES: Cc1nn(C[C@H]2CC[C@H](C(=O)N3OCC[C@H]3c3cc(F)cc(F)c3)CC2)c2cc(C#N)cc(F)c12] | ¹H NMR (400 MHz, DMSO-d₆): δ ppm 8.27 (s, 1 H), 7.32 (d, J = 10.27 Hz, 1 H), 7.11 (tt, J = 9.35, 9.35, 2.32, 2.32 Hz, 1 H), 6.96 (m, 2 H), 5.31 (dd, J = 8.38, 6.42 Hz, 1 H), 4.27 (d, J = 7.09 Hz, 2 H), 4.23 (td, J = 7.86, 7.86, 3.12 Hz, 1 H), 3.83 (m, 1 H), 2.85 (m, 1 H), 2.66 (m, 1 H), 2.59 (s, 3 H), 2.17 (m, 1 H), 1.87 (br d, J = 11.37 Hz, 2 H), 1.68 (br d, J = 11.86 Hz, 1 H), 1.55 (br t, J = 13.20, 13.20 Hz, 2 H), 1.28 (m, 2 H), 1.14 (m, 2 H). LC/MS: m/z [M + H]⁺ 483.3, Rt (I = 220 nm): 2.74 min (LC/MS method A), calc. m/z: 483.51. |
| 102 | 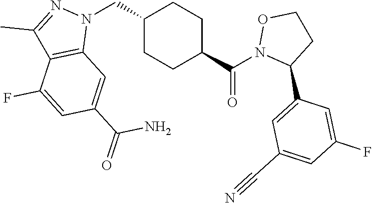 [SMILES: Cc1nn(C[C@H]2CC[C@H](C(=O)N3OCC[C@H]3c3cc(F)cc(C#N)c3)CC2)c2cc(C(N)=O)cc(F)c12] | 1H NMR (400.23 MHz, DMSO-d6) d ppm 8.07 (br s, 1 H), 8.00 (s, 1 H), 7.75 (d, J = 7.81 Hz, 1 H), 7.57 (m, 2 H), 7.45 (br d, J = 9.41 Hz, 1 H), 7.30 (d, J = 11.61 Hz, 1 H), 5.35 (dd, J = 8.31, 6.60 Hz, 1 H), 4.23 (m, 3 H), 3.86 (m, 1 H), 2.87 (dddd, J = 12.12, 9.12, 6.45, 3.00 Hz, 1 H), 2.67 (br d, J = 1.83 Hz, 1 H), 2.58 (s, 3 H), 2.20 (m, 1 H), 1.88 (br d, J = 10.88 Hz, 2 H), 1.70 (br d, J = 11.98 Hz, 1 H), 1.57 (br t, J =14.30, 14.30 Hz, 2 H), 1.26 (m, 2 H), 1.14 (m, 2 H). LC/MS: m/z [M + H]⁺ 508.3, Rt (I = 220 nm): 2.13 min (LC/MS method A), calc. m/z: 508.54. |
| 103 | 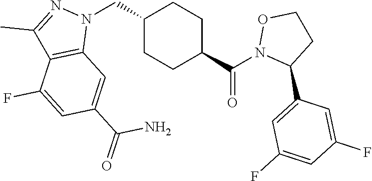 [SMILES: Cc1nn(C[C@H]2CC[C@H](C(=O)N3OCC[C@H]3c3cc(F)cc(F)c3)CC2)c2cc(C(N)=O)cc(F)c12] | 1H NMR (400.23 MHz, DMSO-d6) d ppm 8.07 (br s, 1 H), 8.00 (s, 1 H), 7.56 (br s, 1 H), 7.30 (d, J = 11.74 Hz, 1 H), 7.11 (tt, J = 9.34, 9.34, 2.34, 2.34 Hz, 1 H), 6.96 (m, 2 H), 5.31 (dd, J = 8.56, 6.36 Hz, 1 H), 4.24 (br d, J = 2.93 Hz, 1 H), 4.22 (br d, J = 7.09 Hz, 3 H), 3.83 (m, 1 H), 2.85 (m, 1 H), 2.67 (m, 1 H), 2.58 (s, 3 H), 2.17 (m, 1 H), 1.88 (br d, J = 11.37 Hz, 2 H), 1.69 (br d, J = 12.72 Hz, 1 H), 1.57 (br t, J = 14.61, 14.61 Hz, 2 H), 1.28 (m, 2 H), 1.15 (m, 2 H). LC/MS: m/z [M + H]⁺ 501.3, Rt (I = 220 nm): 2.25 min (LC/MS method A), calc. m/z: 501.52. |
| 104 | 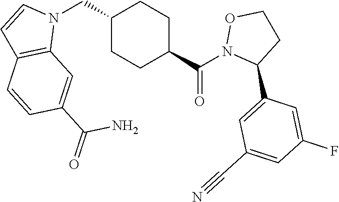 [SMILES: N#Cc1cc(F)cc([C@@H]2CCON2C(=O)[C@H]2CC[C@H](Cn3ccc4ccc(C(N)=O)cc43)CC2)c1] | ¹H NMR (400 MHz, DMSO-d₆): δ ppm 8.06 (s, 1 H), 7.89 (br s, 1 H), 7.75 (d, J = 8.66 Hz, 1 H), 7.53 (m, 5 H), 7.20 (br s, 1 H), 6.47 (d, J = 2.69 Hz, 1 H), 5.35 (dd, J = 8.44, 6.48 Hz, 1 H), 4.25 (td, J = 7.73, 7.73, 2.87 Hz, 1 H), 4.08 (br d, J = 7.21 Hz, 2 H), 3.85 (m, 1 H), 2.87 (m, 1 H), 2.68 (m, 1 H), 2.21 (m, 1 H), 1.86 (m, 2 H), 1.70 (br d, J = 12.84 Hz, 1 H), 1.57 (m, 2 H), 1.28 (m, 2 H), 1.14 (m, 2 H). LC/MS: m/z [M + H]⁺ 475.3, Rt (I = 220 nm): 2.13 min (LC/MS method A), calc. m/z: 475.53. |
| 105 | 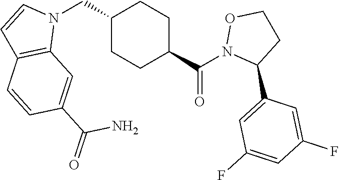 [SMILES: NC(=O)c1ccc2ccn(C[C@H]3CC[C@H](C(=O)N4OCC[C@H]4c4cc(F)cc(F)c4)CC3)c2c1] | ¹H NMR (400 MHz, DMSO-d₆): δ ppm 8.06 (s, 1 H), 7.89 (br s, 1 H), 7.56 (m, 2 H), 7.49 (d, J = 3.18 Hz, 1 H), 7.20 (br s, 1 H), 7.11 (m, 1 H), 6.95 (br d, J = 6.72 Hz, 2 H), 6.47 (d, J = 2.81 Hz, 1 H), 5.31 (m, 1 H), 4.23 (td, J = 7.79, 7.79, 3.00 Hz, 1 H), 4.07 (d, J = 7.21 Hz, 2 H), 3.84 (m, 1 H), 2.85 (m, 1 H), 2.67 (br d, J = 1.71 Hz, 1 H), 2.17 (m, 1 H), 1.88 (br d, J = 12.47 Hz, 2 H), 1.69 (br d, J = 11.86 Hz, 1 H), 1.57 (br t, J = 15.16, 15.16 Hz, 2 H), 1.28 (m, 2 H), 1.13 (m, 2 H). LC/MS: m/z [M + H]⁺ 468.3, Rt (I = 220 nm): 2.25 min (LC/MS method A), calc. m/z: 468.52. |
| 106 | 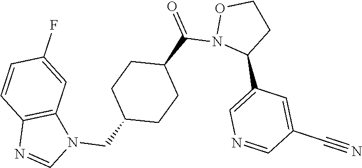 [SMILES: N#Cc1cncc([C@@H]2CCON2C(=O)[C@H]2CC[C@H](Cn3cnc4ccc(F)cc43)CC2)c1] | 1H NMR (400.23 MHz, DMSO-d6) d ppm 8.99 (s, 1 H), 8.93 (d, J = 1.83 Hz, 1 H), 8.76 (d, J = 2.08 Hz, 1 H), 8.17 (t, J = 1.96, 1.96 Hz, 1 H), 7.85 (dd, J = 9.11, 2.26 Hz, 1 H), 7.80 (dd, J = 8.93, 4.65 Hz, 1 H), 7.30 (td, J = 9.29, 9.29, 2.32 Hz, 1 H), 5.39 (dd, J = 8.31, 6.60 Hz, 1 H), 4.28 (td, J = 7.70, 7.70, 3.06 Hz, 2 H), 4.22 (br d, J = 7.09 Hz, 4 H), 2.89 (dddd, J = 12.18, 9.12, 6.33, 3.18 Hz, 1 H), 2.70 (m, 1 H), 2.29 (m, 1 H), 1.89 (br d, J = 11.49 Hz, 2 H), 1.73 (br d, J = 12.59 Hz, 1 H), 1.63 (br s, 2H), 1.30 (m, 2H), 1.14 (m 2H). LC/MS: m/z [M + H]⁺ 434.2, Rt (I = 220 nm): 1.46 min (LC/MS method A), calc. m/z 434.49. |
| 107 | 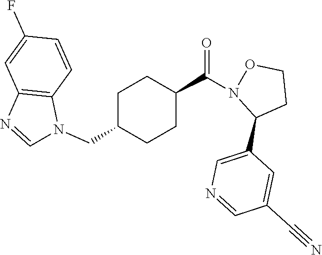 [SMILES: N#Cc1cncc([C@@H]2CCON2C(=O)[C@H]2CC[C@H](Cn3cnc4cc(F)ccc43)CC2)c1] | 1H NMR (400.23 MHz, DMSO-d6) d ppm 8.93 (m, 2 H), 8.76 (d, J = 2.08 Hz, 1 H), 8.17 (m, 1 H), 7.90 (dd, J = 8.93, 4.65 Hz, 1 H), 7.61 (dd, J = 9.11, 2.26 Hz, 1 H), 7.34 (td, J = 9.23, 9.23, 2.20 Hz, 1 H), 5.39 (m, 1 H), 4.26 (m, 4 H), 3.92 (br d, J = 8.19 Hz, 2 H), 3.88 (br d, J = 8.07 Hz, 3 H), 2.89 (dddd, J = 12.13, 9.11, 6.45, 3.12 Hz, 1 H), 2.69 (m, 2 H), 2.29 (m, 1 H), 1.89 (br d, J = 11.25 Hz, 2 H), 1.73 (br d, J = 12.35 Hz, 1 H), 1.62 (br s, 2 H), 1.31 (m, 2 H), 1.14 (m, 2 H). LC/MS: m/z [M + H]⁺ 434.2, Rt (I = 220 nm): 1.43 min (LC/MS method A), calc. m/z: 434.49. |
| 108 | 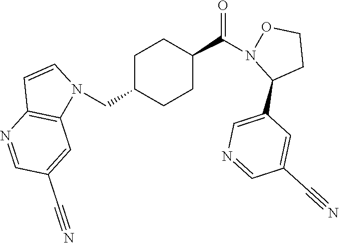 [SMILES: N#Cc1cncc([C@@H]2CCON2C(=O)[C@H]2CC[C@H](Cn3ccc4ncc(C#N)cc43)CC2)c1] | 1H NMR (400.23 MHz, DMSO-d6) d ppm 8.93 (d, J = 1.96 Hz, 1 H), 8.75 (d, J = 2.20 Hz, 1 H), 8.68 (s, 1 H), 8.66 (s, 1 H), 8.17 (t, J = 1.96, 1.96 Hz, 1 H), 8.01 (d, J = 3.18 Hz, 1 H), 6.75 (s, 1 H), 5.39 (dd, J = 8.50, 6.54 Hz, 1 H), 4.28 (td, J = 7.70, 7.70, 3.06 Hz, 1 H), 4.14 (d, J = 7.21 Hz, 2 H), 3.89 (m, 7 H), 2.88 (dddd, J = 12.17, 9.17, 6.48, 3.12 Hz, 1 H), 2.67 (m, 1 H), 2.29 (m, 1 H), 1.85 (m, 2 H), 1.71 (br d, J = 12.10 Hz, 1 H), 1.53 (m, 2 H), 1.27 (m, 2 H), 1.11 (m, 2 H). LC/MS: m/z [M + H]⁺ 441.2, Rt (I = 220 nm): 1.79 min (LC/MS method A), calc. m/z: 441.51. |
| 109 | 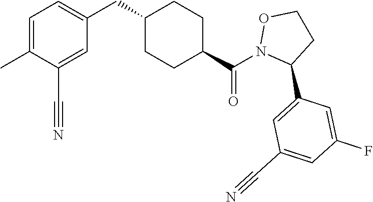 [SMILES: Cc1ccc(C[C@H]2CC[C@H](C(=O)N3OCC[C@H]3c3cc(F)cc(C#N)c3)CC2)cc1C#N] | ¹H NMR (400 MHz, DMSO-d₆): δ ppm 7.77 (m, 1 H), 7.58 (m, 2 H), 7.40 (m, 3 H), 5.35 (dd, J = 8.44, 6.60 Hz, 1 H), 4.25 (td, J = 7.70, 7.70, 3.06 Hz, 1 H), 3.86 (m, 1 H), 2.87 (m, 1 H), 2.65 (m, 1 H), 2.44 (s, 3 H), 2.21 (m, 1 H), 1.87 (br d, J = 12.47 Hz, 1 H), 1.66 (m, 3 H), 1.50 (m, 1 H), 1.28 (m, 2 H), 1.02 (m, 2 H). LC/MS: m/z [M + H]⁺ 432.3, Rt (I = 220 nm): 2.68 min (LC/MS method A), calc. m/z: 432.51. |
| 110 | 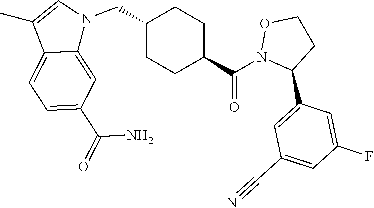 [SMILES: Cc1cn(C[C@H]2CC[C@H](C(=O)N3OCC[C@H]3c3cc(F)cc(C#N)c3)CC2)c2cc(C(N)=O)ccc12] | 1H NMR (400.23 MHz, DMSO-d6) d ppm 8.00 (s, 1 H), 7.87 (br s, 1 H), 7.75 (m, 1 H), 7.58 (m, 1 H), 7.56 (d, J = 1.34 Hz, 1 H), 7.49 (d, J = 8.45 Hz, 1 H), 7.45 (d, J = 9.62 Hz, 1 H), 7.25 (s, 1 H), 7.18 (br s, 1 H), 5.35 (m, 1 H), 4.24 (td, J = 7.70, 7.70, 2.93 Hz, 1 H), 4.00 (br d, J = 7.21 Hz, 2 H), 3.85 (m, 1 H), 2.86 (dddd, J = 12.12, 9.12, 6.45, 3.00 Hz, 1 H), 2.67 (br dd, J = 3.61, 1.90 Hz, 1 H), 2.26 (s, 3 H), 2.20 (m, 1 H), 1.89 (br d, J = 12.47 Hz, 1 H), 1.81 (br d, J = 3.79 Hz, 1 H), 1.70 (br d, J = 11.62 Hz, 1H), 1.58 (m, 2H), 1.27 (m, 2H), 1.11 (m, 2H). LC/MS: m/z [M + H]⁺ 489.3, Rt (I = 220 nm): 2.27 min (LC/MS method A), calc. m/z: 489.56. |
| 111 | 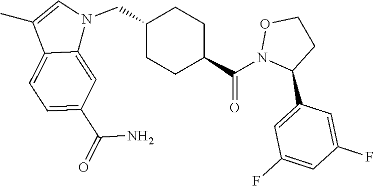 [SMILES: Cc1cn(C[C@H]2CC[C@H](C(=O)N3OCC[C@H]3c3cc(F)cc(F)c3)CC2)c2cc(C(N)=O)ccc12] | 1H NMR (400.23 MHz, DMSO-d6) d ppm 8.00 (s, 1 H), 7.87 (br s, 1 H), 7.56 (d, J = 1.34 Hz, 1 H), 7.49 (m, 1 H), 7.26 (m, 1 H), 7.11 (m, 2 H), 6.95 (br d, J = 6.48 Hz, 2 H), 5.31 (m, 1 H), 4.23 (td, J = 7.73, 7.73, 2.75 Hz, 1 H), 4.00 (br d, J = 7.09 Hz, 2 H), 3.83 (m, 1 H), 2.85 (dddd, J = 12.12, 9.12, 6.45, 3.12 Hz, 1 H), 2.67 (m, 1 H), 2.33 (m, 1 H), 2.26 (s, 3 H), 2.17 (m, 1 H), 1.85 (m, 2 H), 1.69 (br, d, J = 11.62 Hz, 1 H), 1.58 (m, 2 H), 1.28 (m, 2 H), 1.11 (m, 2 H). LC/MS: m/z [M + H]⁺ 482.3, Rt (I = 220 nm): 2.38 min (LC/MS method A), calc. m/z: 482.54. |
| 112 | 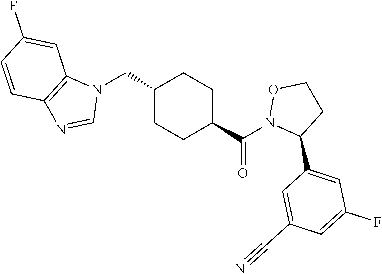 [SMILES: N#Cc1cc(F)cc([C@@H]2CCON2C(=O)[C@H]2CC[C@H](Cn3cnc4ccc(F)cc43)CC2)c1] | ¹H NMR (400 MHz, DMSO-d₆): δ ppm 8.96 (s, 1 H), 7.79 (m, 3 H), 7.58 (s, 1 H), 7.46 (d, J = 9.48 Hz, 1 H), 7.29 (td, J = 9.29, 9.29, 2.32 Hz, 1 H), 5.35 (dd, J = 8.56, 6.48 Hz, 1 H), 4.23 (m, 3 H), 3.86 (m, 1 H), 2.87 (m, 1 H), 2.68 (m, 1 H), 2.21 (m, 1 H), 1.91 (br d, J = 11.74 Hz, 2 H), 1.72 (br d, J = 12.10 Hz, 1 H), 1.63 (m, 2 H), 1.30 (m, 2 H), 1.14 (m, 2 H). LC/MS: m/z [M + H]⁺ 451.3, Rt (I = 220 nm): 1.8 min (LC/MS method A), calc. m/z: 451.49. |
| 113 | 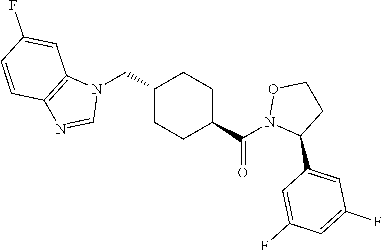 [SMILES: O=C([C@H]1CC[C@H](Cn2cnc3ccc(F)cc32)CC1)N1OCC[C@H]1c1cc(F)cc(F)c1] | ¹H NMR (400 MHz, DMSO-d₆): δ ppm 8.97 (s, 1 H), 7.85 (dd, J = 9.17, 2.20 Hz, 1 H), 7.79 (dd, J = 8.99, 4.71 Hz, 1 H), 7.29 (td, J = 9.29, 9.29, 2.32 Hz, 1 H), 7.12 (tt, J = 9.31, 9.31, 2.31, 2.31 Hz, 1 H), 6.96 (br d, J = 6.48 Hz, 2 H), 5.31 (dd, J = 8.31, 6.48 Hz, 1 H), 4.25 (m, 3 H), 3.85 (m, 1 H), 2.86 (m, 1 H), 2.67 (br d, J = 1.96 Hz, 1 H), 2.18 (m, 1 H), 1.91 (m, 2 H), 1.57 (m, 3 H), 1.30 (m, 2 H), 1.13 (m, 2 H). LC/MS: m/z [M + H]⁺ 444.2, Rt (I = 220 nm): 1.93 min (LC/MS method A), calc. m/z: 444.47. |
| 114 | 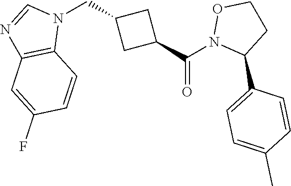 [SMILES: Cc1ccc([C@@H]2CCON2C(=O)[C@H]2C[C@H](Cn3cnc4cc(F)ccc43)C2)cc1] | 1H NMR (400.23 MHz, DMSO-d6) d ppm 8.90 (br s, 1 H), 7.88 (dd, J = 8.93, 4.52 Hz, 1 H), 7.58 (dd, J = 9.23, 2.38 Hz, 1 H), 7.31 (td, J = 9.35, 9.35, 2.45 Hz, 1 H), 7.14 (s, 3 H), 7.14 (m, 1 H), 5.24 (dd, J = 8.68, 6.11 Hz, 1 H), 4.48 (br d, J = 7.70 Hz, 2 H), 4.18 (td, J = 7.70, 7.70, 3.18 Hz, 1 H), 3.76 (m, 7 H), 2.81 (m, 2 H), 2.29 (m, 1 H), 2.27 (s, 5 H), 2.14 (m, 1 H), 2.04 (br dd, J = 15.71, 10.82 Hz, 1 H), 1.97 (br s, 1 H). LC/MS: m/z [M + H]⁺ 394.3, Rt (I = 220 nm): 1.75 min (LC/MS method A), calc. m/z: 394.46. |
| 115 | 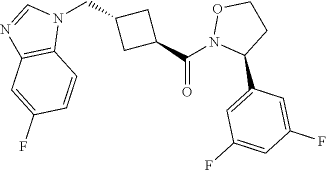 [SMILES: O=C([C@H]1C[C@H](Cn2cnc3cc(F)ccc32)C1)N1OCC[C@H]1c1cc(F)cc(F)c1] | 1H NMR (400.23 MHz, DMSO-d6) d ppm 8.92 (s, 1 H), 7.89 (dd, J = 8.99, 4.58 Hz, 1 H), 7.59 (dd, J = 9.23, 2.38 Hz, 1 H), 7.32 (td, J = 9.29, 9.29, 2.45 Hz, 1 H), 7.13 (tt, J = 9.29, 9.29, 2.32, 2.32 Hz, 1 H), 6.99 (m, 2 H), 5.32 (dd, J = 8.74, 6.30 Hz, 1 H), 4.49 (d, J = 7.83 Hz, 2 H), 4.19 (td, J = 7.73, 7.73, 2.87 Hz, 1 H), 3.78 (ddd, J = 9.41, 7.95, 6.72 Hz, 7 H), 2.84 (m, 2 H), 2.28 (m, 2 H), 2.17 (m, 1 H), 2.07 (m, 1 H), 1.99 (br s, 1 H). LC/MS: m/z [M + H]⁺ 416.2, Rt (I = 220 nm): 1.74 min (LC/MS method A), calc. m/z: 416.42. |
| 116 | 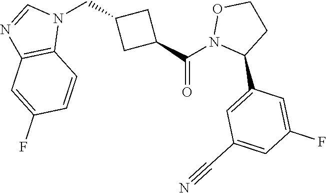 [SMILES: N#Cc1cc(F)cc([C@@H]2CCON2C(=O)[C@H]2C[C@H](Cn3cnc4cc(F)ccc43)C2)c1] | 1H NMR (400.23 MHz, DMSO-d6) d ppm 8.93 (s, 1 H), 7.89 (dd, J = 8.99, 4.58 Hz, 1 H), 7.77 (d, J = 7.94 Hz, 1 H), 7.60 (m, 1 H), 7.49 (br d, J = 9.66 Hz, 1 H), 7.32 (td, J = 9.32, 9.32, 2.38 Hz, 1 H), 5.36 (dd, J = 8.68, 6.48 Hz, 1 H), 4.49 (d, J = 7.82 Hz, 1 H), 4.20 (td, J = 7.73, 7.73, 2.87 Hz, 1 H), 3.81 (m, 2 H), 3.78 (br d, J = 6.72 Hz, 1 H), 2.86 (m, 1 H), 2.25 (m, 2 H), 2.05 (m, 1 H). LC/MS: m/z [M + H]⁺ 423.2, Rt (I = 220 nm): 1.62 min (LC/MS method A), calc. m/z: 423.44. |
| 117 | 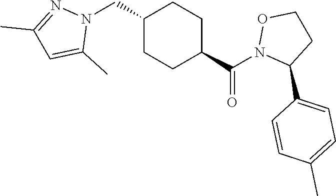 [SMILES: Cc1ccc([C@@H]2CCON2C(=O)[C@H]2CC[C@H](Cn3nc(C)cc3C)CC2)cc1] | 1H NMR (400.23 MHz, DMSO-d6) d ppm 7.13 (s(AA″BB″), 4 H), 5.78 (m, 1 H), 5.24 (dd, J = 8.50, 6.17 Hz, 1 H), 4.22 (td, J = 7.67, 7.67, 3.12 Hz, 1 H), 3.79 (m, 6 H), 2.82 (m, 1 H), 2.67 (br d, J = 1.83 Hz, 1 H), 2.27 (s, 3 H), 2.13 (m, 7 H), 1.84 (br d, J = 12.35 Hz, 1 H), 1.72 (td, J = 7.43, 7.43, 3.73 Hz, 2 H), 1.58 (br d, J = 8.44 Hz, 2 H), 1.27 (m, 2 H), 1.06 (m, 2 H). LC/MS: m/z [M + H]⁺ 382.3, Rt (I = 220 nm): 2.21 min (LC/MS method A), calc. m/z: 382.52. |
| 118 | 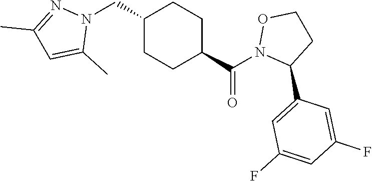 [SMILES: Cc1cc(C)n(C[C@H]2CC[C@H](C(=O)N3OCC[C@H]3c3cc(F)cc(F)c3)CC2)n1] | 1H NMR (400.23 MHz, DMSO-d6) d ppm 7.12 (tt, J = 9.32, 9.32, 2.35, 2.35 Hz, 1 H), 6.96 (br d, J = 6.60 Hz, 2 H), 5.79 (s, 1 H), 5.32 (dd, J = 8.56, 6.36 Hz, 1 H), 4.24 (td, J = 7.76, 7.76, 2.93 Hz, 1 H), 3.84 (m, 1 H), 3.76 (d, J = 7.21 Hz, 4 H), 2.86 (dddd, J = 12.15, 9.12, 6.42, 3.00 Hz, 1 H), 2.67 (br s, 2 H), 2.18 (s, 4 H), 2.18 (m, 1 H), 2.08 (s, 3 H), 1.89 (br d, J = 12.96 Hz, 1 H), 1.73 (m, 2 H), 1.58 (br t, J = 8.13, 8.13 Hz, 2 H), 1.29 (m, 2 H), 1.08 (m, 2 H). LC/MS: m/z [M + H]⁺ 404.3, Rt (I = 220 nm): 2.21 min (LC/MS method A), calc, m/z: 404.47. |
| 119 | 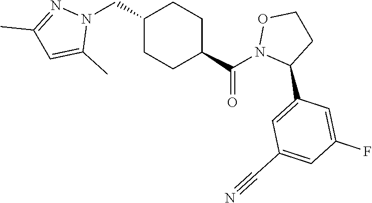 [SMILES: Cc1cc(C)n(C[C@H]2CC[C@H](C(=O)N3OCC[C@H]3c3cc(F)cc(C#N)c3)CC2)n1] | 1H NMR (400.23 MHz, DMSO-d6) d ppm 7.76 (d, J = 8.64 Hz, 1 H), 7.59 (s, 1 H), 7.46 (d, J = 9.51 Hz, 1 H), 5.80 (s, 1 H), 5.36 (dd, J = 8.56, 6.48 Hz, 1 H), 4.25 (td, J = 7.76, 7.76, 2.93 Hz, 1 H), 3.86 (m, 2 H), 3.77 (br d, J = 7.21 Hz, 5 H), 2.88 (dddd, J = 12.17, 9.17, 6.48, 3.00 Hz, 1 H), 2.68 (m, 1 H), 2.22 (m, 1 H), 2.19 (s, 3 H), 2.08 (s, 3 H), 1.90 (br d, J = 12.59 Hz, 1 H), 1.73 (m, 2 H), 1.58 (m, 2 H), 1.28 (m, 2 H), 1.08 (m, 2 H). LC/MS: m/z [M + H]⁺ 411.3, Rt (I = 220 nm): 2.07 min (LC/MS method A), calc. m/z: 412.49. |
| 120 | 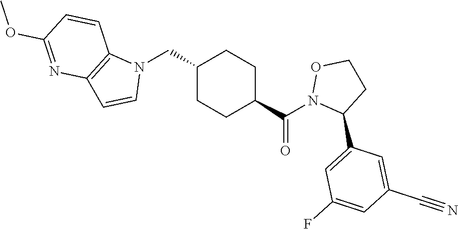 [SMILES: COc1ccc2c(ccn2C[C@H]2CC[C@H](C(=O)N3OCC[C@H]3c3cc(F)cc(C#N)c3)CC2)n1] | ¹H NMR (400 MHz, CDCl₃): δ ppm 7.75 (br d, J = 8.4 Hz, 1 H), 7.39 (s, 1 H), 7.30 (br s, 1 H), 7.27-7.23 (m, 2 H), 6.81-6.64 (m, 2 H), 5.38 (dd, J = 8.7, 6.1 Hz, 1 H), 4.28 (td, J = 7.9, 3.5 Hz, 1 H), 4.09 (s, 3 H), 4.06-3.96 (m, 2 H), 3.88 (dd, J = 15.7, 8.6 Hz, 1 H), 2.96-2.83 (m, 1 H), 2.81-2.68 (m, 1 H), 2.36-2.23 (m, 1 H), 2.08-1.96 (m, 1 H), 1.58-1.38 (m, 5 H), 1.25-0.99 (m, 3 H). LC/MS: m/z [M + H]⁺ 463.2, Rt (I = 220 nm): 0.582 min (LC/MS method B), calc. m/z: 463.72. |
| 121 | 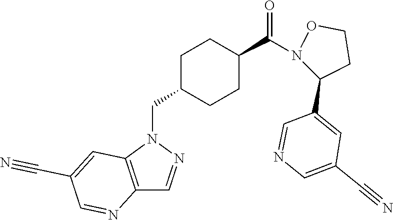 [SMILES: N#Cc1cncc([C@@H]2CCON2C(=O)[C@H]2CC[C@H](Cn3ncc4ncc(C#N)cc43)CC2)c1] | 1H NMR (400.23 MHz, DMSO-d6) d ppm 9.01 (s, 1 H), 8.93 (d, J = 1.96 Hz, 1 H), 8.85 (d, J = 1.71 Hz, 1 H), 8.75 (d, J = 2.08 Hz, 1 H), 8.50 (d, J = 0.86 Hz, 1 H), 8.17 (t, J = 1.96, 1.96 Hz, 1 H), 5.39 (dd, J = 8.56, 6.48 Hz, 1 H), 4.38 (d, J = 7.09 Hz, 2 H), 4.28 (td, J = 7.73, 7.73, 3.12 Hz, 1 H), 3.89 (m, 1 H), 2.88 (dddd, J = 12.12, 9.15, 6.48, 3.06 Hz, 1 H), 2.67 (m, 1 H), 2.28 (m, 1 H), 1.91 (m, 2 H), 1.70 (br d, J = 12.47 Hz, 1 H), 1.54 (br t, J = 12.35, 12.35 Hz, 2 H), 1.27 (m, 2 H), 1.13 (m, 2H). LC/MS: m/z [M + H]⁺ 442.2, Rt (I = 220 nm): 1.79 min (LC/MS method A), calc. m/z: 442.49. |
| 122 | 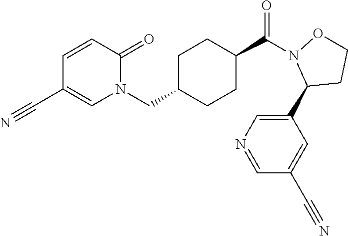 [SMILES: N#Cc1cncc([C@@H]2CCON2C(=O)[C@H]2CC[C@H](Cn3cc(C#N)ccc3=O)CC2)c1] | 1H NMR (400.23 MHz, DMSO-d6) d ppm 8.93 (d, J = 1.96 Hz, 1 H), 8.77 (d, J = 2.08 Hz, 1 H), 8.54 (d, J = 2.32 Hz, 1 H), 8.18 (t, J = 1.96, 1.96 Hz, 1 H), 7.66 (dd, J = 9.54, 2.45 Hz, 1 H), 6.48 (d, J = 9.54 Hz, 1 H), 5.40 (dd, J = 8.44, 6.60 Hz, 1 H), 4.29 (td, J = 7.76, 7.76, 3.06 Hz, 1 H), 3.91 (m, 2 H), 3.78 (d, J = 7.21 Hz, 4 H), 2.89 (dddd, J = 12.15, 9.15, 6.45, 3.18 Hz, 1 H), 2.68 (m, 1 H), 2.29 (m, 1 H), 1.90 (br d, J = 12.59 Hz, 1 H), 1.74 (br dd, J = 7.89, 3.85 Hz, 2 H), 1.60 (br d, J = 12.72 Hz, 2 H), 1.27 (m, 2 H), 1.07 (m, 2H). LC/MS: m/z [M + H]⁺ 418.2, Rt (I = 220 nm): 1.53 min (LC/MS method A), calc. m/z: 418.47. |
| 123 | 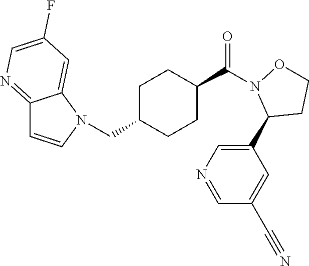 [SMILES: N#Cc1cncc([C@@H]2CCON2C(=O)[C@H]2CC[C@H](Cn3ccc4ncc(F)cc43)CC2)c1] | 1H NMR (400.23 MHz, DMSO-d6) d ppm 8.93 (m, 1 H), 8.76 (m, 1 H), 8.35 (s, 1 H), 8.17 (m, 1 H), 7.70 (d, J = 3.30 Hz, 1 H), 6.60 (d, J = 3.18 Hz, 1 H), 5.39 (m, 1 H), 4.28 (m, 1 H), 4.05 (d, J = 7.34 Hz, 1 H), 3.89 (m, 1 H), 3.21 (br d, J = 6.24 Hz, 3 H), 2.88 (br dd, J = 6.11, 3.06 Hz, 1 H), 2.67 (m, 1 H), 2.31 (m, 2 H), 1.87 (br d, J = 12.84 Hz, 2 H), 1.76 (m, 2 H), 1.69 (br s, 1 H), 1.54 (br t, J = 11.92, 11.92 Hz, 2 H), 1.26 (br dd, J = 16.26, 3.91 Hz, 2 H), 1.09 (m, 2 H). LC/MS: m/z [M + H]⁺ 434.2, Rt (I = 220 nm): 1.63 min (LC/MS method A), calc. m/z: 434.49. |
| 124 | 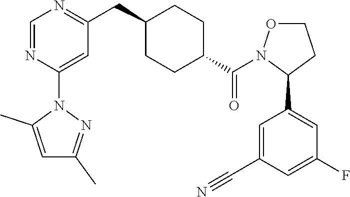 [SMILES: Cc1cc(C)n(-c2cc(C[C@H]3CC[C@H](C(=O)N4OCC[C@H]4c4cc(F)cc(C#N)c4)CC3)ncn2)n1] | 1H NMR (400.23 MHz, DMSO-d6) d ppm 8.92 (d, J = 1.10 Hz, 1 H), 7.76 (d, J = 7.81 Hz, 1 H), 7.72 (s, 1 H), 7.59 (s, 1 H), 7.46 (br d, J = 9.66 Hz, 1 H), 6.21 (s, 1 H), 5.36 (dd, J = 8.56, 6.48 Hz, 1 H), 4.25 (td, J = 7.70, 7.70, 2.93 Hz, 1 H), 3.86 (m, 2 H), 2.87 (dddd, J = 12.15, 9.15, 6.45, 3.06 Hz, 1 H), 2.68 (br d, J = 6.85 Hz, 3 H), 2.66 (s, 3 H), 2.22 (s, 3 H), 2.21 (m, 1 H), 1.89 (br d, J = 12.59 Hz, 1 H), 1.78 (br d, J = 3.18 Hz, 1 H), 1.70 (br d, J = 9.29 Hz, 3 H), 1.31 (m, 3 H), 1.11 (m, 2H). LC/MS: m/z [M + H]⁺ 489.3, Rt (I = 220 nm): 2.59 min (LC/MS method A), calc. m/z: 489.24. |
| 125 | 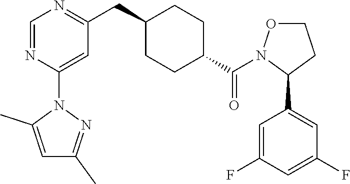 [SMILES: Cc1cc(C)n(-c2cc(C[C@H]3CC[C@H](C(=O)N4OCC[C@H]4c4cc(F)cc(F)c4)CC3)ncn2)n1] | 1H NMR (400.23 MHz, DMSO-6) d ppm 8.92 (d, J = 0.98 Hz, 1 H), 7.72 (d, J = 0.98 Hz, 1 H), 7.12 (tt, J = 9.31, 9.31, 2.37, 2.37 Hz, 1 H), 6.96 (m, 2 H), 6.21 (s, 1 H), 5.32 (dd, J = 8.62, 6.42 Hz, 1 H), 4.24 (td, J = 7.73, 7.73, 2.87 Hz, 1 H), 3.84 (m, 1 H), 2.86 (dddd, J = 12.12, 9.15, 6.48, 3.06 Hz, 1 H), 2.67 (m, 6 H), 2.22 (s, 3 H), 2.17 (m, 1 H), 1.89 (br d, J = 13.20 Hz, 1 H), 1.77 (br d, J = 3.55 Hz, 1 H), 1.69 (br d, J = 8.68 Hz, 3 H), 1.32 (q, J = 12.59, 12.59, 12.59 Hz, 2 H), 1.12 (m, 2 H). LC/MS: m/z [M + H]⁺ 482.3, Rt (I = 220 nm): 2.72 min (LC/MS method A), calc. m/z: 482.24. |
| 126 | 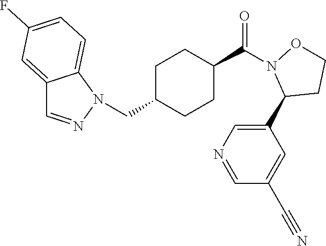 [SMILES: N#Cc1cncc([C@@H]2CCON2C(=O)[C@H]2CC[C@H](Cn3ncc4cc(F)ccc43)CC2)c1] | 1H NMR (400.23 MHz, DMSO-d6) d ppm 8.93 (d, J = 1.83 Hz, 1 H), 8.75 (d, J = 2.20 Hz, 1 H), 8.17 (t, J = 1.96, 1.96 Hz, 1 H), 8.04 (d, J = 0.73 Hz, 1 H), 7.74 (dd, J = 9.05, 4.16 Hz, 1 H), 7.52 (dd, J = 9.23, 2.26 Hz, 1 H), 7.27 (td, J = 9.14, 9.14, 2.51 Hz, 1 H), 5.38 (dd, J = 8.50, 6.42 Hz, 1 H), 4.27 (m, 3 H), 3.89 (m, 1 H), 2.88 (dddd, J = 12.13, 9.14, 6.42, 3.12 Hz, 1 H), 2.67 (s, 1 H), 2.28 (m, 1 H), 1.86 (br d, J = 11.98 Hz, 2 H), 1.69 (br d, J = 11.49 Hz, 1 H), 1.55 (br t, J = 11.43, 11.43 Hz, 2 H), 1.30-1.05 (m, 4H). LC/MS: m/z [M + H]⁺ 434.2, Rt (I = 220 nm): 2.15 min (LC/MS method A), calc. m/z: 434.49. |
| 127 | 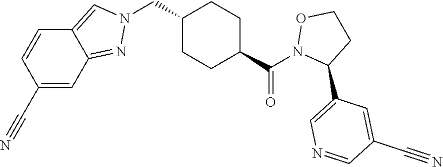 [SMILES: N#Cc1cncc([C@@H]2CCON2C(=O)[C@H]2CC[C@H](Cn3cc4ccc(C#N)cc4n3)CC2)c1] | 1H NMR (400.23 MHz, DMSO-d6) d ppm 8.93 (d, J = 1.96 Hz, 1 H), 8.75 (d, J = 2.20 Hz, 1 H), 8.40 (s, 1 H), 8.27 (s, 1 H), 8.17 (t, J = 1.96, 1.96 Hz, 1 H), 7.92 (d, J = 8.0 Hz, 1 H), 7.71 (dd, J = 8.74, 1.53 Hz, 1 H), 5.38 (dd, J = 8.50, 6.54 Hz, 1 H), 4.34 (d, J = 6.97 Hz, 2 H), 4.27 (td, J = 7.73, 7.73, 3.00 Hz, 1 H), 3.88 (m, 1 H), 2.88 (dddd, J = 12.18, 9.19, 6.51, 3.06 Hz, 1 H), 2.66 (m, 1 H), 2.28 (m, 1 H), 1.87 (m, 2 H), 1.69 (br d, J = 13.08 Hz, 1 H), 1.55 (br t, J = 12.10, 12.10, Hz, 2 H), 1.25 (m, 2H), 1.12 (m, 2H). LC/MS: m/z [M + H]⁺ 441.2, Rt (I = 220 nm): 1.99 min (LC/MS method A), calc. m/z: 441.51. |
| 128 | 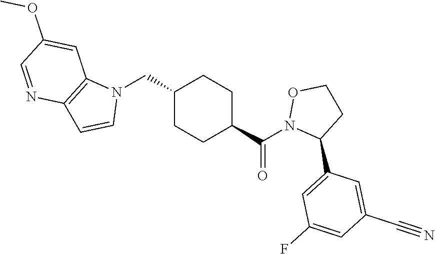 [SMILES: COc1cnc2ccn(C[C@H]3CC[C@H](C(=O)N4OCC[C@H]4c4cc(F)cc(C#N)c4)CC3)c2c1] | ¹H NMR: (400 MHz, CDCl₃): δ ppm 8.45 (s, 1 H), 7.73 (s, 1 H), 7.75 (s, 1 H), 7.39 (s, 1 H), 7.25 (m, 1 H), 6.97 (br s, 1 H), 5.39 (m, 1 H), 4.30 (m, 1 H), 4.11 (m, 2 H), 4.04 (s, 3 H), 3.91 (m, 1 H), 2.92 (m, 1 H), 2.78 (m, 1 H), 2.30 (m, 1 H), 2.08 (m, 1 H), 1.95-1.67 (m, 4 H), 1.52 (m, 2 H), 1.17 (m, 2 H). LC/MS: m/z [M + H]⁺ 463.2, Rt (I = 220 nm): 0.77 min (LC/MS method B), calc. m/z: 463.22. |
| 129 | 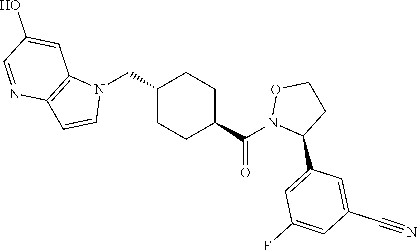 [SMILES: N#Cc1cc(F)cc([C@@H]2CCON2C(=O)[C@H]2CC[C@H](Cn3ccc4ncc(O)cc43)CC2)c1] | ¹H (400 MHz, DMSO-d6) δ = 9.40 (br s, 1H), 7.98 (d, J = 2.2 Hz, 1H), 7.80-7.69 (m, 1H), 7.58 (s, 1H), 7.49- 7.40 (m, 1H), 7.37 (d, J = 3.2 Hz, 1H), 7.19 (d, J = 1.7 Hz, 1H), 6.40 (d, J = 2.7 Hz, 1H), 5.42-5.25 (m, 1H), 4.30-4.19 (m, 1H), 3.93 (br d, J = 7.1 Hz, 2H), 3.89-3.79 (m, 1H), 2.92-2.80 (m, 1H), 2.74-2.59 (m, 1H), 2.29-2.14 (m, 1H), 1.94-1.81 (m, 1H), 1.81- 1.63 (m, 2H), 1.63-1.48 (m, 2H), 1.34-1.18 (m, 2H), 1.17-0.97 (m, 2H). LC/MS: m/z [M + H]+ 449.3, Rt (I = 220 nm): 0.804 min (LC/MS method B), calc. m/z: 449.20. |
| 130 | 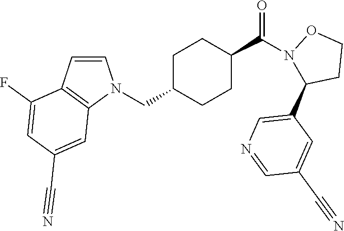 [SMILES: N#Cc1cncc([C@@H]2CCON2C(=O)[C@H]2CC[C@H](Cn3ccc4c(F)cc(C#N)cc43)CC2)c1] | ¹H NMR (400 MHz, DMSO-d₆): δ ppm 8.93 (d, J = 1.83 Hz, 1 H), 8.76 (d, J = 2.08 Hz, 1 H), 8.17 (t, J = 1.96, 1.96 Hz, 1 H), 8.12 (s, 1 H), 7.72 (d, J = 3.18 Hz, 1 H), 7.28 (dd, J = 10.27, 0.86 Hz, 1 H), 6.67 (d, J = 2.57 Hz, 1 H), 5.39 (dd, J = 8.44, 6.36 Hz, 1 H), 4.28 (td, J = 7.73, 7.73, 3.12 Hz, 1 H), 4.14 (d, J = 7.21 Hz, 2 H), 3.89 (m, 1 H), 2.88 (m, 1 H), 2.67 (m, 1 H), 2.28 (m, 1 H), 1.86 (br t, J = 11.62, 11.62 Hz, 2 H), 1.82 (br s, 1 H), 1.54 (m, 2 H), 1.28 (m, 2 H), 1.12 (m, 2 H). LC/MS: m/z [M + H]⁺ 458.2, Rt (I = 220 nm): 2.37 min (LC/MS method A), calc. m/z: 458.51. |
| 131 | 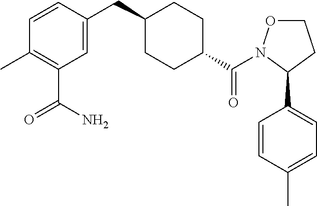 [SMILES: Cc1ccc([C@@H]2CCON2C(=O)[C@H]2CC[C@H](Cc3ccc(C)c(C(N)=O)c3)CC2)cc1] | ¹H NMR (400 MHz, DMSO-d₆): δ ppm 7.63 (br s, 1 H), 7.28 (br s, 1 H), 7.09 (m, 7 H), 5.24 (dd, J = 8.31, 6.36 Hz, 1 H), 4.21 (td, J = 7.52, 7.52, 2.69 Hz, 1 H), 3.82 (m, 1 H), 2.81 (m, 1 H), 2.66 (m, 1 H), 2.43 (br d, J = 6.85 Hz, 2 H), 2.31 (s, 3 H), 2.26 (s, 3 H), 2.14 (m, 1 H), 1.82 (br d, J = 12.10 Hz, 1 H), 1.68 (br d, J = 12.23 Hz, 3 H), 1.47 (br s, 1 H), 1.28 (m, 2 H), 1.01 (m, 2 H). LC/MS: m/z [M + H]⁺ 421.3, Rt (I = 220 nm): 2.33 min (LC/MS method A), calc. m/z: 421.25. |
| 132 | 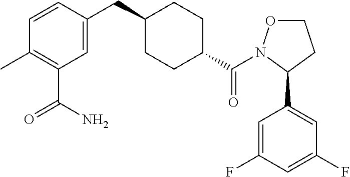 [SMILES: Cc1ccc(C[C@H]2CC[C@H](C(=O)N3OCC[C@H]3c3cc(F)cc(F)c3)CC2)cc1C(N)=O] | ¹H NMR (400 MHz, DMSO-d₆): δ ppm 7.63 (br s, 1 H), 7.28 (br s, 1 H), 7.11 (m, 4 H), 6.96 (m, 2 H), 5.31 (dd, J = 8.62, 6.30 Hz, 1 H), 4.23 (td, J = 7.76, 7.76, 2.93 Hz, 1 H), 3.83 (m, 1 H), 2.85 (m, 1 H), 2.67 (br d, J = 1.96 Hz, 1 H), 2.44 (d, J = 6.97 Hz, 2 H), 2.31 (s, 3 H), 2.17 (m, 1 H), 1.87 (br d, J = 12.59 Hz, 1 H), 1.68 (br d, J = 8.68 Hz, 3 H), 1.48 (br s, 1 H), 1.29 (m, 2 H), 1.02 (m, 2 H). LC/MS: m/z [M + H]⁺ 443.3, Rt (I = 220 nm): 2.33 min (LC/MS method A), calc. m/z: 443.22. |
| 133 | 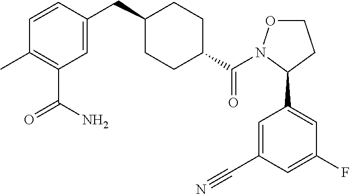 [SMILES: Cc1ccc(C[C@H]2CC[C@H](C(=O)N3OCC[C@H]3c3cc(F)cc(C#N)c3)CC2)cc1C(N)=O] | 1H NMR (400.23 MHz, DMSO-d6) d ppm 7.76 (br d, J = 8.56 Hz, 1 H), 7.60 (m, 2 H), 7.46 (br d, J = 9.66 Hz, 1 H), 7.28 (br s, 1 H), 7.11 (m, 3 H), 5.35 (m, 1 H), 4.25 (td, J = 7.64, 7.64, 2.81 Hz, 2 H), 3.97 (br s, 7 H), 3.86 (m, 4 H), 2.87 (dddd, J = 12.07, 9.08, 6.42, 3.12 Hz, 1 H), 2.67 (br s, 1 H), 2.43 (s, 2 H), 2.31 (s, 3 H), 2.21 (m, 1 H), 1.87 (br d, J = 11.37 Hz, 1 H), 1.69 (br d, J = 9.54 Hz, 3 H), 1.48 (br s, 1 H), 1.29 (m, 2 H), 1.01 (dq, J = 12.72, 12.55, 12.55, 12.55 Hz, 2 H). LC/MS: m/z [M + H]⁺ 450.2, Rt (I = 220 nm): 2.22 min (LC/MS method A), calc. m/z: 450.22. |
| 134 | 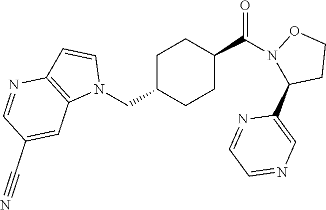 [SMILES: N#Cc1cnc2ccn(C[C@H]3CC[C@H](C(=O)N4OCC[C@H]4c4cnccn4)CC3)c2c1] | 1H NMR (400.23 MHz, DMSO-d6) d ppm 8.61 (m, 5 H), 8.00 (d, J = 3.18 Hz, 1 H), 6.74 (d, J = 2.57 Hz, 1 H), 5.39 (dd, J = 8.68, 6.24 Hz, 1 H), 4.29 (td, J = 7.64, 7.64, 3.42 Hz, 1 H), 4.14 (d, J = 7.21 Hz, 2 H), 3.92 (m, 1 H), 2.80 (m, 1 H), 2.67 (m, 1 H), 1.82 (br d, J = 11.49 Hz, 2 H), 1.71 (br d, J = 11.98 Hz, 1 H), 1.50 (br s, 2 H), 1.26 (m, 2 H), 1.09 (m, 2 H). LC/MS: m/z [M + H]⁺ 417.2, Rt (I = 220 nm): 1.64 min (LC/MS method A), calc. m/z: 417.20. |
| 135 | 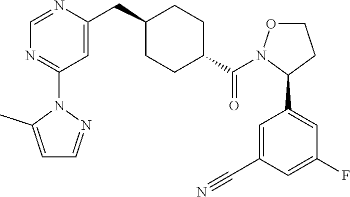 [SMILES: Cc1ccnn1-c1cc(C[C@H]2CC[C@H](C(=O)N3OCC[C@H]3c3cc(F)cc(C#N)c3)CC2)ncn1] | ¹H NMR (400 MHz, DMSO-d₆): δ ppm 9.19 (s, 1 H), 8.72 (d, J = 2.0 Hz, 1 H), 8.39 (s, 1 H), 8.01 (d, J = 2.0 Hz, 1 H), 7.13 (tt, J = 2.3, 9.3 Hz, 1 H), 6.96 (br d, J = 6.6 Hz, 2 H), 5.32 (dd, J = 6.6, 8.2 Hz, 1 H), 4.24 (dt, J = 2.8, 7.7 Hz, 1 H), 3.87-3.81 (m, 1 H), 2.93-2.77 (m, 1 H), 2.87 (s, 3 H), 2.71 (m, 1 H), 2.50 (m, 2 H), 2.22- 2.13 (m, 1 H), 1.91-1.84 (m, 2 H), 1.69-1.61 (m, 3 H), 1.36-1.27 (m, 2 H), 1.16-1.05 (m, 2 H). LC/MS: m/z [M + H]⁺ 475.3, Rt (I = 220 nm): 1.47 min (LC/MS method A), calc. m/z: 475.23. |
| 136 | 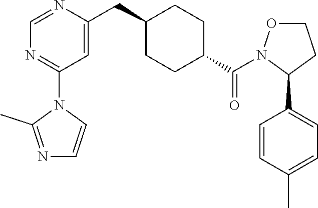 [SMILES: Cc1ccc([C@@H]2CCON2C(=O)[C@H]2CC[C@H](Cc3cc(-n4ccnc4C)ncn3)CC2)cc1] | 1H NMR (400.23 MHz, DMSO-d6) d ppm 9.19 (d, J = 0.86 Hz, 1 H), 8.16 (d, J = 2.20 Hz, 1 H), 7.87 (s, 1 H), 7.70 (d, J = 1.96 Hz, 1 H), 7.13 (s(AA″BB″), 4 H), 5.24 (m, 1 H), 4.23 (td, J = 7.64, 7.64, 3.06 Hz, 1 H), 3.83 (br dd, J = 8.62, 7.52 Hz, 3 H), 3.79 (br s, 2 H), 2.84 (s, 4 H), 2.79 (m, 2 H), 2.27 (s, 3 H), 2.15 (m, 1 H), 1.85 (br d, J = 10.88 Hz, 2 H), 1.72 (br d, J = 12.35 Hz, 3 H), 1.33 (m, 2 H), 1.13 (m, 2 H). LC/MS: m/z [M + H]⁺ 446.3, Rt (I = 220 nm): 1.58 min (LC/MS method A), calc. m/z: 446.26. |
| 137 | 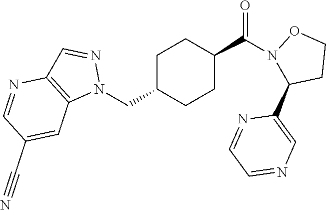 [SMILES: N#Cc1cnc2cnn(C[C@H]3CC[C@H](C(=O)N4OCC[C@H]4c4cnccn4)CC3)c2c1] | 1H NMR (400.23 MHz, DMSO-d6) d ppm 9.01 (s, 1 H), 8.85 (d, J = 1.71 Hz, 1 H), 8.58 (m, 3 H), 8.50 (d, J = 0.73 Hz, 1 H), 5.39 (dd, J = 8.80, 6.11 Hz, 1 H), 4.37 (d, J = 7.09 Hz, 2 H), 4.28 (m, 1 H), 3.93 (m, 2 H), 3.83 (m, 1 H), 3.20 (br d, J = 6.24 Hz, 1 H), 2.81 (m, 1 H), 2.66 (m, 2 H), 1.92 (m, 1 H), 1.82 (br d, J = 12.72 Hz, 1 H), 1.71 (br d, J = 13.94 Hz, 1 H), 1.54 (br d, J = 12.96 Hz, 2 H), 1.27 (m, 3 H), 1.12 (m, 2 H). LC/MS: m/z [M + H]⁺ 418.2, Rt (I = 220 nm): 1.64 min (LC/MS method A), calc. m/z: 418.20. |
| 138 | 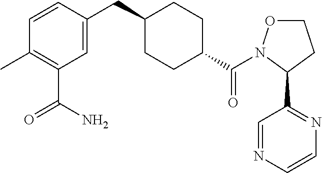 [SMILES: Cc1ccc(C[C@H]2CC[C@H](C(=O)N3OCC[C@H]3c3cnccn3)CC2)cc1C(N)=O] | 1H NMR (400.23 MHz, DMSO-d6) d ppm 8.58 (m, 3 H), 7.63 (br s, 1 H), 7.28 (br s, 1 H), 7.10 (m, 3 H), 5.39 (dd, J = 8.62, 6.17 Hz, 1 H), 4.30 (td, J = 7.58, 7.58, 3.42 Hz, 1 H), 3.92 (m, 1 H), 2.81 (m, 1 H), 2.65 (m, 1 H), 2.44 (br d, J = 6.97 Hz, 2 H), 2.31 (s, 3 H), 1.82 (br d, J = 12.23 Hz, 1 H), 1.69 (br d, J = 11.86 Hz, 3 H), 1.47 (br s, 1 H), 1.28 (m, 2 H), 1.00 (m, 2 H). LC/MS: m/z [M + H]⁺ 409.3, Rt (I = 220 nm): 1.76 min (LC/MS method A), calc. m/z: 409.22. |
| 139 | 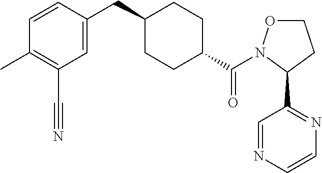 [SMILES: Cc1ccc(C[C@H]2CC[C@H](C(=O)N3OCC[C@H]3c3cnccn3)CC2)cc1C#N] | ¹H NMR (400 MHz, DMSO-d₆): δ ppm 8.58 (m, 3 H), 7.55 (s, 1 H), 7.38 (m, 2 H), 5.39 (dd, J = 8.74, 6.17 Hz, 1 H), 4.29 (td, J = 7.61, 7.61, 3.48 Hz, 1 H), 3.92 (m, 1 H), 2.81 (m, 1 H), 2.64 (br t, J = 11.55, 11.55 Hz, 1 H), 2.44 (s, 3 H), 1.81 (br d, J = 12.10 Hz, 1 H), 1.71 (br s, 1 H), 1.64 (br d, J = 12.84 Hz, 2 H), 1.48 (m, 1 H), 1.27 (m, 2 H), 1.00 (m, 2 H). LC/MS: m/z [M + H]⁺ 391.3, Rt (I = 220 nm): 2.26 min (LC/MS method A), calc. m/z: 391.21. |
| 140 | 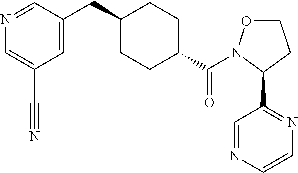 [SMILES: N#Cc1cncc(C[C@H]2CC[C@H](C(=O)N3OCC[C@H]3c3cnccn3)CC2)c1] | 1H NMR (400.23 MHz, DMSO-d6) d ppm 8.85 (d, J = 1.83 Hz, 1 H), 8.69 (d, J = 1.96 Hz, 1 H), 8.58 (m, 3 H), 8.16 (s, 1 H), 5.40 (dd, J = 8.62, 6.17 Hz, 1 H), 4.30 (td, J = 7.64, 7.64, 3.67 Hz, 1 H), 3.93 (m, 1 H), 2.81 (m, 1 H), 2.64 (m, 1 H), 2.57 (d, J = 6.97 Hz, 2 H), 1.83 (br d, J = 12.23 Hz, 1 H), 1.71 (br d, J = 12.96 Hz, 1 H), 1.64 (br d, J = 12.59 Hz, 2 H), 1.56 (m, 1 H), 1.29 (m, 2 H), 1.04 (m, 2 H). LC/MS: m/z [M + H]⁺ 378.2, Rt (I = 220 nm): 1.7 min (LC/MS method A), calc. m/z: 378.19. |
| 141 | 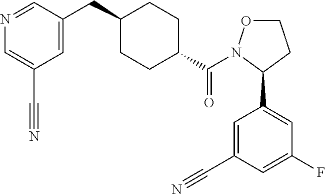 [SMILES: N#Cc1cncc(C[C@H]2CC[C@H](C(=O)N3OCC[C@H]3c3cc(F)cc(C#N)c3)CC2)c1] | 1H NMR (400.23 MHz, DMSO-d6) d ppm 8.85 (d, J = 1.96 Hz, 1 H), 8.69 (d, J = 1.96 Hz, 1 H), 8.16 (t, J = 1.89, 1.89 Hz, 1 H), 7.76 (d, J = 8.68 Hz, 1 H), 7.59 (s, 1 H), 7.46 (br d, J = 9.66 Hz, 1 H), 5.36 (dd, J = 8.44, 6.60 Hz, 1 H), 4.25 (td, J = 7.76, 7.76, 2.93 Hz, 1 H), 3.86 (m, 1 H), 2.87 (dddd, J = 12.10, 9.11, 6.48, 3.00 Hz, 1 H), 2.67 (br t, J = 11.62, 11.62 Hz, 1 H), 2.57 (d, J = 6.85 Hz, 2 H), 2.21 (m, 1 H), 1.88 (br d, J = 12.35 Hz, 1 H), 1.62 (m, 4 H), 1.30 (m, 2 H), 1.05 (m, 2 H). LC/MS: m/z [M + H]⁺ 419.2, Rt (I = 220 nm): 2.19 min (LC/MS method A), calc. m/z: 419.19. |
| 142 | 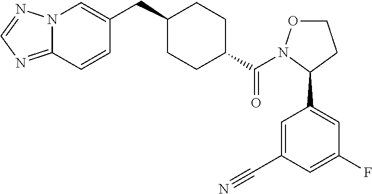 [SMILES: N#Cc1cc(F)cc([C@@H]2CCON2C(=O)[C@H]2CC[C@H](Cc3ccc4ncnn4c3)CC2)c1] | ¹H NMR (400 MHz, DMSO-d₆): δ ppm 8.78 (s, 1 H), 8.45 (s, 1 H), 7.77 (t, J = 8.28, 8.28 Hz, 2 H), 7.59 (s, 1 H), 7.56 (d, J = 8.92 Hz, 1 H), 7.46 (br d, J = 9.66 Hz, 1 H), 5.36 (m, 1 H), 4.46 (m, 1 H), 3.86 (m, 1 H), 2.87 (m, 1 H), 2.68 (br s, 1 H), 2.59 (d, J = 6.97 Hz, 2 H), 2.21 (m, 1 H), 1.89 (br d, J = 12.59 Hz, 1 H), 1.70 (br d, J = 9.41 Hz, 3 H), 1.60 (m, 1 H), 1.31 (m, 2 H), 1.06 (m, 2 H). LC/MS: m/z [M + H]⁺ 434.2, Rt (I = 220 nm): 2.03 min (LC/MS method A), calc. m/z: 434.20. |
| 143 | 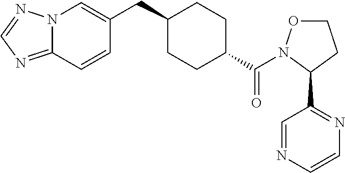 [SMILES: O=C([C@H]1CC[C@H](Cc2ccc3ncnn3c2)CC1)N1OCC[C@H]1c1cnccn1] | 1H NMR (400.23 MHz, DMSO-d6) d ppm 8.78 (s, 1 H), 8.58 (m, 3 H), 8.44 (s, 1 H), 7.77 (d, J = 9.05 Hz, 1 H), 7.56 (dd, J = 9.05, 1.47 Hz, 1 H), 5.40 (dd, J = 8.68, 6.24 Hz, 1 H), 4.30 (td, J = 7.64, 7.64, 3.30 Hz, 1 H), , 3.90 (br s, 7 H), 2.81 (m, 1 H), 2.65 (m, 1 H), 2.59 (br d, J = 6.97 Hz, 3 H), 1.83 (br d, J = 12.35 Hz, 1 H), 1.70 (br d, J = 10.88 Hz, 3 H), 1.59 (br d, J = 2.93 Hz, 1 H), 1.30 (m, 2 H), 1.05 (m, 2 H). LC/MS: m/z [M + H]⁺ 393.2, Rt (I = 220 nm): 1.55 min (LC/MS method A), calc. m/z: 393.20. |
| 144 | 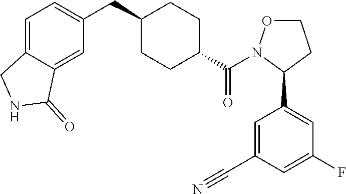 [SMILES: N#Cc1cc(F)cc([C@@H]2CCON2C(=O)[C@H]2CC[C@H](Cc3ccc4c(c3)C(=O)NC4)CC2)c1] | ¹H NMR (400 MHz, DMSO-d₆): δ ppm 8.47 (s, 1 H), 7.75 (br d, J = 8.44 Hz, 1 H), 7.58 (s, 1 H), 7.43 (m, 4 H), 5.35 (m, 1 H), 4.32 (s, 2 H), 4.25 (td, J = 7.70, 7.70, 2.93 Hz, 1 H), 3.85 (m, 1 H), 2.87 (m, 1 H), 2.66 (m, 1 H), 2.59 (br d, J = 6.97 Hz, 2 H), 2.21 (m, 1 H), 1.88 (br d, J = 12.84 Hz, 1 H), 1.68 (m, 3 H), 1.52 (br s, 1 H), 1.29 (m, 2 H), 1.03 (m, 2 H). LC/MS: m/z [M + H]⁺ 448.2, Rt (I = 220 nm): 2.12 min (LC/MS method A), calc. m/z: 448.20. |
| 145 | 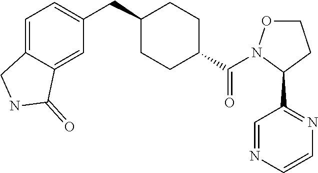 [SMILES: O=C1NCc2ccc(C[C@H]3CC[C@H](C(=O)N4OCC[C@H]4c4cnccn4)CC3)cc21] | 1H NMR (400.23 MHz, DMSO-d6) d ppm 8.58 (m, 3 H), 8.47 (s, 1 H), 7.42 (m, 3 H), 5.39 (dd, J = 8.80, 6.11 Hz, 1 H), 4.32 (s, 2 H), 4.29 (m, 1 H), 4.02 (br s, 7 H), 3.93 (br dd, J = 8.44, 7.21 Hz, 4 H), 3.89 (br s, 2 H), 2.81 (m, 1 H), 2.65 (m, 1 H), 2.58 (br d, J = 6.97 Hz, 2 H), 1.82 (br d, J = 11.86 Hz, 1 H), 1.67 (br d, J = 12.72 Hz, 3 H), 1.51 (m, 1 H), 1.28 (m, 2 H), 1.02 (m, 2 H). LC/MS: m/z [M + H]⁺ 407.2, Rt (I = 220 nm): 1.69 min (LC/MS method A), calc. m/z: 407.21. |
| 146 | 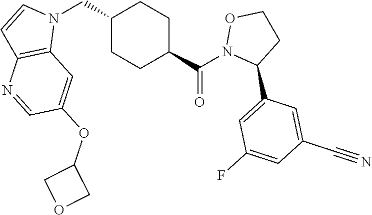 [SMILES: N#Cc1cc(F)cc([C@@H]2CCON2C(=O)[C@H]2CC[C@H](Cn3ccc4ncc(OC5COC5)cc43)CC2)c1] | 1H NMR (400 MHz, CHLOROFORM-d) δ = 8.27 (d, J = 2.1 Hz, 1H), 7.65 (d, J = 1.6 Hz, 1H), 7.56 (d, J = 3.4 Hz, 1H), 7.38 (s, 1H), 7.27-7.21 (m, 2H), 7.00 (d, J = 3.0 Hz, 1H), 5.44-5.33 (m, 2H), 5.10 (t, J = 6.9 Hz, 2H), 4.81 (dd, J = 4.8, 7.7 Hz, 2H), 4.28 (dt, J = 3.1, 7.8 Hz, 1H), 4.15-4.01 (m, 2H), 3.93-3.83 (m, 1H), 2.97- 2.83 (m, 1H), 2.83-2.70 (m, 1H), 2.37-2.22 (m, 1H), 2.12-2.00 (m, 1H), 1.96-1.87 (m, 1H), 1.86- 1.75 (m, 2H), 1.74-1.66 (m, 1H), 1.58-1.42 (m, 2H), 1.25-1.05 (m, 2H). LC/MS: m/z [M + H]⁺ 505.3, Rt (I = 220 nm): 0.799 min (LC/MS method B), calc. m/z: 505.23. |
| 147 | 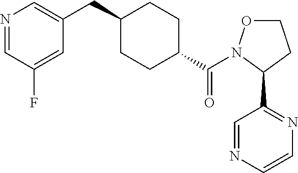 [SMILES: O=C([C@H]1CC[C@H](Cc2cncc(F)c2)CC1)N1OCC[C@H]1c1cnccn1] | 1H NMR (400.23 MHz, DMSO-d6) d ppm 8.58 (m, 3 H), 8.41 (d, J = 2.69 Hz, 1 H), 8.29 (s, 1 H), 7.60 (br d, J = 9.66 Hz, 1 H), 5.40 (dd, J = 8.74, 6.17 Hz, 1 H), 4.62 (br s, 5 H), 4.30 (td, J = 7.67, 7.67, 3.48 Hz, 3 H), 3.93 (m, 1 H), 2.81 (m, 1 H), 2.64 (m, 1 H), 2.55 (s, 1 H), 1.83 (br d, J = 12.35 Hz, 1 H), 1.73 (br s, 1 H), 1.66 (br d, J = 13.45 Hz, 2 H), 1.56 (br d, J = 3.55 Hz, 1 H), 1.29 (m, 2 H), 1.03 (m, 2 H). LC/MS: m/z [M + H]⁺ 371.2, Rt (I = 220 nm): 1.74 min (LC/MS method A), calc. m/z: 371.19. |
| 148 | 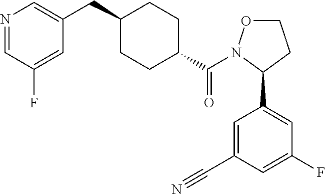 [SMILES: N#Cc1cc(F)cc([C@@H]2CCON2C(=O)[C@H]2CC[C@H](Cc3cncc(F)c3)CC2)c1] | ¹H NMR (400 MHz, DMSO-d₆): δ ppm 8.41 (d, J = 2.57 Hz, 1 H), 8.29 (s, 1 H), 7.76 (br d, J = 8.80 Hz, 1 H), 7.59 (m, 2 H), 7.46 (br d, J = 9.54 Hz, 1 H), 5.36 (m, 1 H), 4.26 (m, 1 H), 3.86 (m, 1 H), 2.87 (m, 1 H), 2.67 (br s, 1 H), 2.56 (m, 2 H), 2.21 (m, 1 H), 1.89 (br d, J = 12.47 Hz, 1 H), 1.66 (br d, J = 12.96 Hz, 3 H), 1.56 (br s, 1 H), 1.30 (m, 2 H), 1.03 (m, 2 H). LC/MS: m/z [M + H]⁺ 412.2, Rt (I = 220 nm): 2.25 min (LC/MS method A), calc. m/z: 412.18. |
| 149 | 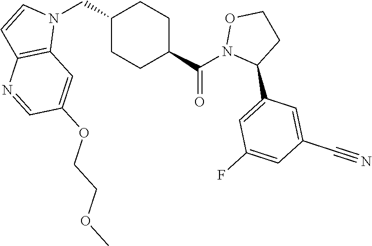 [SMILES: COCCOc1cnc2ccn(C[C@H]3CC[C@H](C(=O)N4OCC[C@H]4c4cc(F)cc(C#N)c4)CC3)c2c1] | 1H NMR (400 MHz, CHLOROFORM-d) δ = 8.40 (s, 1H), 7.90 (s, 1H), 7.58 (d, J = 3.4 Hz, 1H), 7.39 (s, 1H), 7.27-7.21 (m, 2H), 7.01 (s, 1H), 5.41-5.37 (m, 1H), 4.35-4.29 (m, 3H), 4.11-4.07 (m, 2H), 3.93- 3.85 (m, 2H), 3.48 (s, 3H), 2.92-2.89 (m, 1H), 2.78- 2.76 (m, 1H), 2.37-2.26 (m, 1H), 2.12-2.00 (m, 1H), 1.90-1.6 (m, 4H), 1.51-1.48 (m, 2H), 1.25-1.4 (m, 2H). LC/MS: m/z [M + H]⁺ 507.3, Rt (I = 220 nm): 0.813 min (LC/MS method B), calc. m/z: 507.24. |
| 150 | 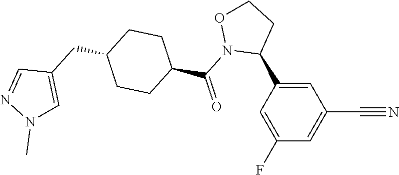 [SMILES: Cn1cc(C[C@H]2CC[C@H](C(=O)N3OCC[C@H]3c3cc(F)cc(C#N)c3)CC2)cn1] | 1H NMR (400.23 MHz, DMSO-d6) d ppm 7.76 (br d, J = 8.56 Hz, 1 H), 7.59 (s, 1 H), 7.46 (d, J = 9.53 Hz, 1 H), 7.42 (s, 1 H), 7.18 (s, 1 H), 5.36 (m, 1 H), 4.25 (td, J = 7.76, 7.76, 2.69 Hz, 1 H), 3.93 (br s, 1 H), 3.85 (m, 3 H), 3.76 (s, 9 H), 2.87 (m, 1 H), 2.65 (m, 2 H), 2.27 (d, J = 6.72 Hz, 2 H), 2.22 (m, 1 H), 1.88 (br d, J = 11.98 Hz, 1 H), 1.72 (m, 3 H), 1.30 (m, 3 H), 0.97 (m, 2 H). LC/MS: m/z [M + H]⁺ 397.2, Rt (I = 220 nm): 2.14 min (LC/MS method A), calc. m/z: 397.20. |
| 151 | 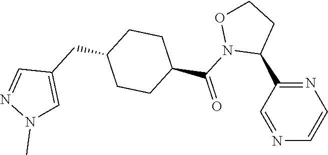 [SMILES: Cn1cc(C[C@H]2CC[C@H](C(=O)N3OCC[C@H]3c3cnccn3)CC2)cn1] | 1H NMR (400.23 MHz, DMSO-d6) d ppm 8.59 (m, 3 H), 7.42 (s, 1 H), 7.19 (s, 1 H), 5.40 (dd, J = 8.80, 6.11 Hz, 1 H), 4.30 (td, J = 7.67, 7.67, 3.61 Hz, 4 H), 4.08 (br s, 12 H), 3.94 (br dd, J = 8.44, 7.21 Hz, 4 H), 3.90 (br s, 1 H), 3.76 (s, 3 H), 2.81 (m, 1 H), 2.63 (br t, J = 12.04, 12.04 Hz, 1 H), 2.27 (d, J = 6.72 Hz, 2 H), 1.82 (br d, J = 12.72 Hz, 1 H), 1.72 (br d, J = 11.62 Hz, 3 H), 1.29 (m, 3 H), 0.96 (m, 2 H). LC/MS: m/z [M + H]⁺ 356.2, Rt (I = 220 nm): 1.63 min (LC/MS method A), calc. m/z: 356.21. |
| 152 | 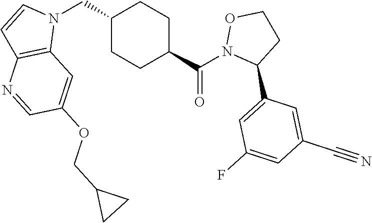 [SMILES: N#Cc1cc(F)cc([C@@H]2CCON2C(=O)[C@H]2CC[C@H](Cn3ccc4ncc(OCC5CC5)cc43)CC2)c1] | ¹H NMR (400 MHz, CDCl₃): δ ppm 8.28 (br s, 1 H), 7.67 (br s, 1 H), 7.46 (br d, J = 4.3 Hz, 1 H), 7.30 (s, 1 H), 7.18-7.13 (m, 2 H), 6.99-6.92 (m, 1 H), 5.34-5.27 (m, 1 H), 4.25-4.16 (m, 1 H), 4.06-3.90 (m, 4 H), 3.85- 3.77 (m, 1 H), 2.82 (br d, J = 2.6 Hz, 1 H), 2.70 (br d, J = 10.9 Hz, 1 H), 2.28-2.19 (m, 1 H), 2.01-1.95 (m, 1 H), 1.85-1.62 (m, 4 H), 1.48-1.37 (m, 2 H), 1.26 (br s, 1 H), 1.16-1.01 (m, 2 H), 0.66 (br d, J = 7.6 Hz, 2 H), 0.36 (br d, J = 4.1 Hz, 2 H). LC/MS: m/z [M + H]⁺ 503.2, Rt (I = 220 nm): 0.853 min (LC/MS method B), calc. m/z: 503.25. |
| 153 | 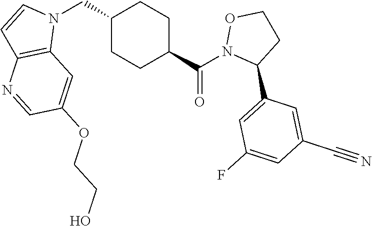 [SMILES: N#Cc1cc(F)cc([C@@H]2CCON2C(=O)[C@H]2CC[C@H](Cn3ccc4ncc(OCCO)cc43)CC2)c1] | ¹H NMR (400 MHz, CDCl₃): δ ppm 8.30 (br s, 1 H), 7.39 (s, 1 H), 7.28-7.19 (m, 4 H), 6.67 (d, J = 3.3 Hz, 1 H), 5.39 (dd, J = 8.6, 6.2 Hz, 1 H), 4.28 (td, J = 7.9, 2.9 Hz, 1 H), 4.25-4.20 (m, 2 H), 4.07-4.03 (m, 2 H), 3.95 (t, J = 7.1 Hz, 2 H), 3.91-3.84 (m, 1 H), 2.95-2.83 (m, 1 H), 2.82-2.71 (m, 1 H), 2.36-2.22 (m, 1 H), 2.08-2.00 (m, 1 H), 1.93-1.78 (m, 4 H), 1.49 (m, 2 H), 1.24-1.08 (m, 2 H). LC/MS: m/z [M + H]⁺ 493.3, Rt (I = 220 nm): 0.689 min (LC/MS method B), calc. m/z: 493.23. |
| 154 | 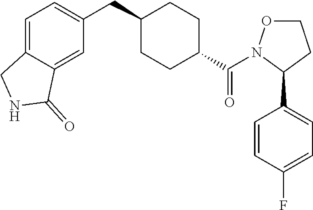 [SMILES: O=C1NCc2ccc(C[C@H]3CC[C@H](C(=O)N4OCC[C@H]4c4ccc(F)cc4)CC3)cc21] | 1H NMR (400.23 MHz, DMSO-d6) d ppm 8.47 (s, 1 H), 7.42 (m, 3 H), 7.29 (dd, J = 8.50, 5.56 Hz, 2 H), 7.15 (t, J = 8.26, 8.26 Hz, 2 H), 5.29 (br t, J = 7.34, 7.34 Hz, 1 H), 4.32 (s, 2 H), 4.23 (td, J = 7.70, 7.70, 2.93 Hz, 1 H), 3.83 (m, 2 H), 3.79 (br s, 1 H), 2.83 (m, 1 H), 2.66 (br d, J = 9.66 Hz, 1 H), 2.58 (br d, J = 6.85 Hz, 2 H), 2.15 (m, 1 H), 1.84 (br d, J = 12.59 Hz, 1 H), 1.67 (br d, J = 12.23 Hz, 3 H), 1.51 (br s, 1 H), 1.28 (m, 2 H), 1.03 (m, 2 H). LC/MS: m/z [M + H]⁺ 423.2, Rt (I = 220 nm): 2.18 min (LC/MS method A), calc. m/z: 423.21. |
| 155 | 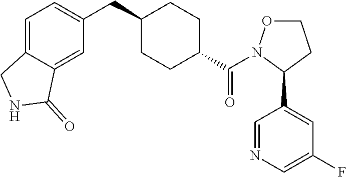 [SMILES: O=C1NCc2ccc(C[C@H]3CC[C@H](C(=O)N4OCC[C@H]4c4cncc(F)c4)CC3)cc21] | 1H NMR (400.23 MHz, DMSO-d6) d ppm 8.48 (m, 2 H), 8.38 (s, 1 H), 7.57 (dt, J = 9.75, 2.03, 2.03 Hz, 1 H), 7.42 (m, 3 H), 5.38 (dd, J = 8.44, 6.60 Hz, 1 H), 4.50 (br s, 1 H), 4.32 (s, 3 H), 4.27 (td, J = 7.73, 7.73, 3.00 Hz, 5 H), 3.88 (br dd, J = 8.68, 7.34 Hz, 3 H), 3.84 (s, 1 H), 2.88 (dddd, J = 12.09, 9.12, 6.54, 3.06 Hz, 1 H), 2.66 (m, 1 H), 2.58 (d, J = 6.97 Hz, 2 H), 2.25 (m, 1 H), 1.86 (br d, J = 12.23 Hz, 1 H), 1.68 (br d, J = 10.64 Hz, 3 H), 1.51 (m, 1 H), 1.28 (q, J = 12.23, 2H), 1.03 (bt, J = 12.53, 2H). LC/MS: m/z [M + H]⁺ 424.2, Rt (I = 220 nm): 1.87 min (LC/MS method A), calc. m/z: 424.20. |
| 156 | 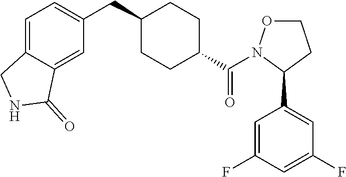 [SMILES: O=C1NCc2ccc(C[C@H]3CC[C@H](C(=O)N4OCC[C@H]4c4cc(F)cc(F)c4)CC3)cc21] | ¹H NMR (400 MHz, DMSO-d₆): δ ppm 8.47 (s, 1 H), 7.42 (m, 3 H), 7.11 (t, J = 9.32, 9.32 Hz, 1 H), 6.96 (br d, J = 6.60 Hz, 2 H), 5.31 (m, 1 H), 4.32 (s, 2 H), 4.23 (td, J = 7.70, 7.70, 2.93 Hz, 1 H), 3.83 (m, 1 H), 2.85 (m, 1 H), 2.66 (br s, 1 H), 2.58 (br d, J = 6.97 Hz, 2 H), 2.17 (m, 1 H), 1.87 (br d, J = 12.35 Hz, 1 H), 1.67 (m, 3 H), 1.52 (br s, 1 H), 1.29 (m, 2 H), 1.03 (m, 2 H). LC/MS: m/z [M + H]⁺ 441.2, Rt (I = 220 nm): 2.27 min (LC/MS method A), calc. m/z: 441.20. |
| 157 | 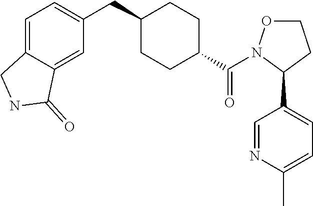 [SMILES: Cc1ccc([C@@H]2CCON2C(=O)[C@H]2CC[C@H](Cc3ccc4c(c3)C(=O)NC4)CC2)cn1] | 1H NMR (400.23 MHz, DMSO-d6) d ppm 8.57 (s, 1 H), 8.48 (s, 1 H), 8.07 (br d, J = 7.58 Hz, 1 H), 7.67 (d, J = 8.31 Hz, 1 H), 7.43 (m, 3 H), 5.41 (m, 1 H), 4.49 (br s, 1 H), 4.32 (s, 3 H), 4.28 (m, 3 H), 4.09 (br s, 3 H), 3.91 (br dd, J = 8.74, 7.27 Hz, 3 H), 2.89 (dddd, J = 12.09, 9.09, 6.39, 3.00 Hz, 1 H), 2.61 (s, 4 H), 2.60 (m, 2 H), 2.26 (m, 1 H), 1.84 (br d, J = 11.86 Hz, 1 H), 1.68 (br d, J = 10.76 Hz, 3 H), 1.51 (br s, 1 H), 1.27 (m, 2 H), 1.03 (m, 2 H). LC/MS: m/z [M + H]⁺ 420.3, Rt (I = 220 nm): 1.41 min (LC/MS method A), calc. m/z: 420.23. |
| 158 | 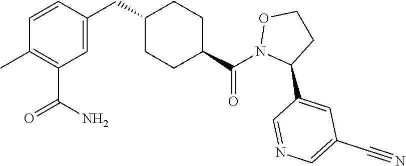 [SMILES: Cc1ccc(C[C@H]2CC[C@H](C(=O)N3OCC[C@H]3c3cncc(C#N)c3)CC2)cc1C(N)=O] | 1H NMR (400.23 MHz, DMSO-d6) d ppm 8.93 (d, J = 1.96 Hz, 1 H), 8.76 (d, J = 2.20 Hz, 1 H), 8.18 (t, J = 1.89, 1.89 Hz, 1 H), 7.62 (br s, 1 H), 7.28 (br s, 1 H), 7.10 (m, 3 H), 5.39 (dd, J = 8.50, 6.54 Hz, 1 H), 4.28 (td, J = 7.67, 7.67, 2.87 Hz, 7 H), 3.89 (m, 1 H), 2.88 (m, 1 H), 2.67 (br t, J = 11.49, 11.49 Hz, 1 H), 2.44 (br d, J = 6.85 Hz, 2 H), 2.31 (s, 3 H), 2.27 (m, 1 H), 1.86 (br d, J = 11.49 Hz, 1 H), 1.69 (br d, J = 10.76 Hz, 3 H), 1.48 (br s, 1 H), 1.28 (m, 2 H), 1.01 (m, 2 H). LC/MS: m/z [M + H]⁺ 433.2, Rt (I = 220 nm): 1.9 min (LC/MS method A), calc. m/z: 433.22. |
| 159 | 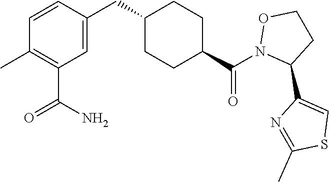 [SMILES: Cc1nc([C@@H]2CCON2C(=O)[C@H]2CC[C@H](Cc3ccc(C)c(C(N)=O)c3)CC2)cs1] | 1H NMR (400.23 MHz, DMSO-d6) d ppm 7.63 (br s, 1 H), 7.28 (br s, 1 H), 7.18 (s, 1 H), 7.10 (m, 3 H), 5.35 (dd, J = 8.50, 5.69 Hz, 1 H), 4.31 (br s, 5 H), 4.22 (td, J = 7.58, 7.58, 3.91 Hz, 4 H), 3.84 (q, J = 7.82, 7.82, 7.82 Hz, 1 H), 2.69 (m, 1 H), 2.62 (s, 4 H), 2.41 (m, 3 H), 2.31 (s, 3 H), 1.81 (br d, J = 11.98 Hz, 1 H), 1.68 (br d, J = 11.49 Hz, 3 H), 1.47 (br s, 1 H), 1.28 (m, 2 H), 1.00 (m, 2 H). LC/MS: m/z [M + H]⁺ 428.2, Rt (I = 220 nm): 1.95 min (LC/MS method A), calc. m/z: 428.20. |
| 160 | 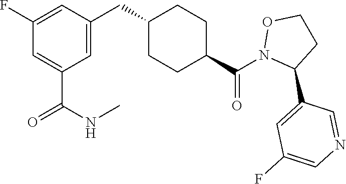 [SMILES: CNC(=O)c1cc(F)cc(C[C@H]2CC[C@H](C(=O)N3OCC[C@H]3c3cncc(F)c3)CC2)c1] | 1H NMR (400.23 MHz, DMSO-d6) d ppm 8.48 (d, J = 2.69 Hz, 1 H), 8.46 (br d, J = 4.40 Hz, 1 H), 8.38 (s, 1 H), 7.57 (br d, J = 9.66 Hz, 1 H), 7.49 (s, 1 H), 7.42 (br d, J = 9.66 Hz, 1 H), 7.18 (br d, J = 9.66 Hz, 1 H), 5.39 (m, 1 H), 4.27 (td, J = 7.73, 7.73, 2.87 Hz, 5 H), 3.88 (m, 3 H), 2.88 (m, 1 H), 2.78 (d, J = 4.52 Hz, 3 H), 2.68 (m, 1 H), 2.26 (m, 1 H), 1.87 (br d, J = 12.35 Hz, 1 H), 1.67 (br d, J = 12.23 Hz, 2 H), 1.55 (br s, 1 H), 1.30 (m, 2 H), 1.02 (q, J = 12.55 Hz, 2H). LC/MS: m/z [M + H]⁺ 444.2, Rt (I = 220 nm): 2.02 min (LC/MS method A), calc. m/z: 444.21. |
| 161 | 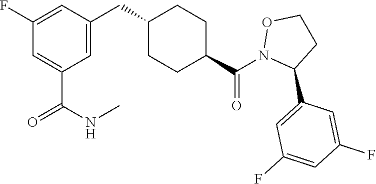 [SMILES: CNC(=O)c1cc(F)cc(C[C@H]2CC[C@H](C(=O)N3OCC[C@H]3c3cc(F)cc(F)c3)CC2)c1] | ¹H NMR (400 MHz, DMSO-d₆): δ ppm 8.46 (br d, J = 4.40 Hz, 1 H), 7.49 (s, 1 H), 7.42 (br d, J = 9.66 Hz, 1 H), 7.18 (br d, J = 9.54 Hz, 1 H), 7.12 (br t, J = 9.02, 9.02 Hz, 1 H), 6.96 (br d, J = 6.60 Hz, 2 H), 5.32 (m, 1 H), 4.23 (td, J = 7.67, 7.67, 2.87 Hz, 1 H), 3.84 (m, 1 H), 2.85 (m, 1 H), 2.78 (d, J = 4.52 Hz, 3 H), 2.67 (br s, 1 H), 2.17 (m, 1 H), 1.88 (br d, J = 12.59 Hz, 1 H), 1.67 (br s, 3 H), 1.56 (br s, 1 H), 1.31 (m, 2 H), 1.04 (m, 2 H). LC/MS: m/z [M + H]⁺ 461.2, Rt (I = 220 nm): 2.42 min (LC/MS method A), calc. m/z: 461.21. |
| 162 | 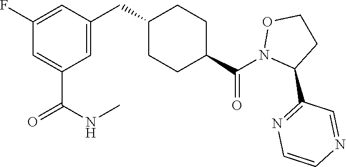 [SMILES: CNC(=O)c1cc(F)cc(C[C@H]2CC[C@H](C(=O)N3OCC[C@H]3c3cnccn3)CC2)c1] | 1H NMR (400.23 MHz, DMSO-d6) d ppm 8.58 (m, 3 H), 8.46 (br d, J = 4.40 Hz, 1 H), 7.49 (s, 1 H), 7.42 (br d, J = 9.05 Hz, 1 H), 7.18 (br d, J = 9.17 Hz, 1 H), 5.40 (dd, J = 8.80, 6.11 Hz, 1 H), 4.30 (td, J = 7.58, 7.58, 3.42 Hz, 3 H), 4.08 (br s, 5 H), 3.93 (m, 3 H), 2.82 (m, 1 H), 2.77 (d, J = 4.52 Hz, 3 H), 2.65 (m, 1 H), 1.83 (br d, J = 12.10 Hz, 1 H), 1.73 (br s, 1 H), 1.67 (br d, J = 13.94 Hz, 2 H), 1.54 (br d, J = 7.21 Hz, 1 H), 1.29 (m, 2 H), 1.03 (m, 2 H). LC/MS: m/z [M + H]⁺ 427.2, Rt (I = 220 nm): 1.84 min (LC/MS method A), calc. m/z: 427.22. |
| 163 | 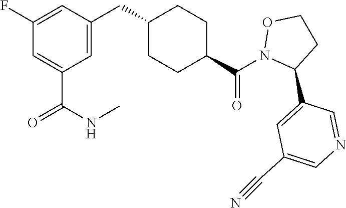 [SMILES: CNC(=O)c1cc(F)cc(C[C@H]2CC[C@H](C(=O)N3OCC[C@H]3c3cncc(C#N)c3)CC2)c1] | 1H NMR (400.23 MHz, DMSO-d6) d ppm 8.93 (d, J = 1.83 Hz, 1 H), 8.76 (d, J = 2.08 Hz, 1 H), 8.46 (br d, J = 4.40 Hz, 1 H), 8.18 (t, J = 1.90, 1.90 Hz, 1 H), 7.49 (s, 1 H), 7.42 (br d, J = 9.66 Hz, 1 H), 7.18 (br d, J = 9.66 Hz, 1 H), 5.39 (dd, J = 8.44, 6.60 Hz, 1 H), 4.28 (td, J = 7.73, 7.73, 3.00 Hz, 1 H), 2.89 (m, 1 H), 2.78 (d, J = 4.52 Hz, 3 H), 2.67 (m, 1 H), 2.29 (m, 1 H), 1.87 (br d, J = 13.45 Hz, 1 H), 1.68 (m, 3 H), 1.54 (br d, J = 6.85 Hz, 1 H), 1.29 (m, 2 H), 1.03 (m, 2 H). LC/MS: m/z [M + H]⁺ 451.2, Rt (I = 220 nm): 1.98 min (LC/MS method A), calc. m/z: 451.22. |
| 164 | 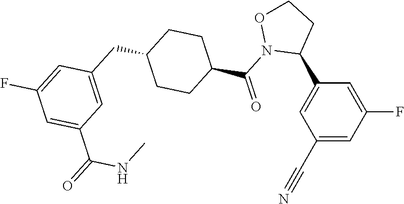 [SMILES: CNC(=O)c1cc(F)cc(C[C@H]2CC[C@H](C(=O)N3OCC[C@H]3c3cc(F)cc(C#N)c3)CC2)c1] | ¹H NMR (400 MHz, DMSO-d₆): δ ppm 8.46 (br d, J = 4.40 Hz, 1 H), 7.76 (br d, J = 8.56 Hz, 1 H), 7.59 (s, 1 H), 7.49 (s, 1 H), 7.44 (m, 2 H), 7.18 (br d, J = 9.54 Hz, 1 H), 5.36 (m, 1 H), 4.25 (td, J = 7.70, 7.70, 2.93 Hz, 1 H), 3.86 (m, 1 H), 2.87 (m, 1 H), 2.78 (d, J = 4.52 Hz, 3 H), 2.67 (m, 1 H), 2.21 (m, 1 H), 1.88 (br d, J = 13.20 Hz, 1 H), 1.68 (br s, 3 H), 1.56 (br s, 1 H), 1.30 (m, 2 H), 1.03 (m, 2 H). LC/MS: m/z [M + H]⁺ 468.2, Rt (I = 220 nm): 2.29 min (LC/MS method A), calc. m/z: 468.21. |
| 165 | 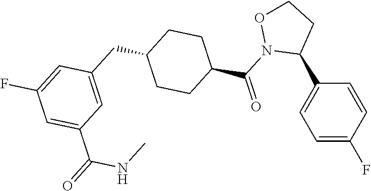 [SMILES: CNC(=O)c1cc(F)cc(C[C@H]2CC[C@H](C(=O)N3OCC[C@H]3c3ccc(F)cc3)CC2)c1] | ¹H NMR (400 MHz, DMSO-d₆): δ ppm 8.46 (br d, J = 4.28 Hz, 1 H), 7.49 (s, 1 H), 7.42 (br d, J = 9.17 Hz, 1 H), 7.29 (dd, J = 8.44, 5.62 Hz, 2 H), 7.16 (m, 3 H), 5.29 (br t, J = 7.40, 7.40 Hz, 1 H), 4.23 (m, 1 H), 3.83 (m, 1 H), 2.84 (m, 1 H), 2.78 (d, J = 4.40 Hz, 3 H), 2.67 (br s, 1 H), 2.16 (m, 1 H), 1.84 (br d, J = 12.10 Hz, 1 H), 1.66 (m, 3 H), 1.55 (br s, 1 H), 1.30 (m, 2 H), 1.03 (m, 2 H). LC/MS: m/z [M + H]⁺ 443.2, Rt (I = 220 nm): 2.33 min (LC/MS method A), calc. m/z: 443.22. |
| 166 | 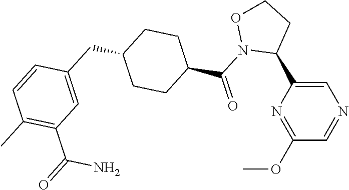 [SMILES: COc1cncc([C@@H]2CCON2C(=O)[C@H]2CC[C@H](Cc3ccc(C)c(C(N)=O)c3)CC2)n1] | 1H NMR (400.23 MHz, DMSO-d6) d ppm 8.20 (s, 1 H), 8.11 (s, 1 H), 7.62 (br s, 1 H), 7.28 (br s, 1 H), 7.10 (m, 3 H), 5.31 (dd, J = 8.74, 5.56 Hz, 1 H), 4.28 (td, J = 7.55, 7.55, 4.34 Hz, 1 H), 4.07 (br s, 1 H), 3.94 (m, 3 H), 3.89 (s, 5 H), , 2.78 (m, 1 H), 2.64 (m, 1 H), 2.44 (br d, J = 6.85 Hz, 2 H), 2.31 (s, 3 H), 1.84 (br d, J = 11.49 Hz, 1 H), 1.69 (br d, J = 10.64 Hz, 3 H), 1.47 (br s, 1 H), 1.29 (m, 2 H), 1.00 (m, 2 H). LC/MS: m/z [M + H]⁺ 439.2, Rt (I = 220 nm): 2.01 min (LC/MS method A), calc. m/z: 439.24. |
| 167 | 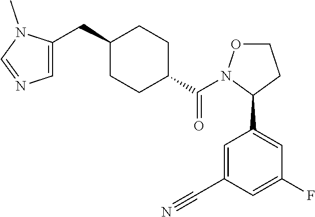 [SMILES: Cn1cncc1C[C@H]1CC[C@H](C(=O)N2OCC[C@H]2c2cc(F)cc(C#N)c2)CC1] | 1H NMR (400.23 MHz, DMSO-d6) d ppm 8.97 (s, 1 H), 7.77 (br d, J = 8.56 Hz, 1 H), 7.59 (s, 1 H), 7.47 (m, 2 H), 5.36 (m, 1 H), 4.26 (m, 1 H), 3.87 (m, 1 H), 3.76 (s, 3 H), 2.89 (ddd, J = 6.24, 3.00, 2.75 Hz, 1 H), 2.69 (br d, J = 14.55 Hz, 1 H), 2.57 (s, 2 H), 2.22 (m, 1 H), 1.91 (br d, J = 12.59 Hz, 1 H), 1.75 (m, 3 H), 1.60 (br s, 1 H), 1.37 (m, 2 H), 1.10 (m, 2 H). LC/MS: m/z [M + H]⁺ 397.2, Rt (I = 220 nm): 1.39 min (LC/MS method A), calc. m/z: 397.20. |
| 168 | 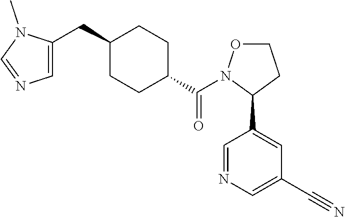 [SMILES: Cn1cncc1C[C@H]1CC[C@H](C(=O)N2OCC[C@H]2c2cncc(C#N)c2)CC1] | 1H NMR (400.23 MHz, DMSO-d6) d ppm 14.03 (br s, 1 H), 8.95 (d, J = 9.50 Hz, 2 H), 8.77 (d, J = 2.08 Hz, 1 H), 8.18 (t, J = 1.83, 1.83 Hz, 1 H), 7.47 (s, 1 H), 5.40 (m, 1 H), 4.29 (td, J = 7.70, 7.70, 3.06 Hz, 1 H), 3.91 (m, 1 H), 3.76 (s, 3 H), 3.58 (br s, 3 H), 2.90 (dddd, J = 12.12, 9.09, 6.42, 3.06 Hz, 1 H), 2.70 (m, 1 H), 2.57 (s, 1 H), 2.30 (m, 1 H), 1.90 (br d, J = 11.86 Hz, 1 H), 1.76 (br d, J = 10.64 Hz, 3 H), 1.60 (br s, 1 H), 1.38 (s, 2 H), 1.35 (m, 1 H), 1.09 (m, 2 H). LC/MS: m/z [M + H]⁺ 380.2, Rt (I = 220 nm): 1.09 min (LC/MS method A), calc. m/z: 380.21. |
| 169 | 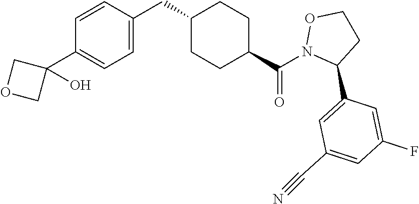 [SMILES: N#Cc1cc(F)cc([C@@H]2CCON2C(=O)[C@H]2CC[C@H](Cc3ccc(C4(O)COC4)cc3)CC2)c1] | 1H NMR (400.23 MHz, DMSO-d6) d ppm 7.76 (br d, J = 8.44 Hz, 1 H), 7.58 (s, 1 H), 7.49 (d, J = 8.19 Hz, 3 H), 7.18 (d, J = 8.19 Hz, 2 H), 5.35 (dd, J = 8.38, 6.66 Hz, 1 H), 4.74 (d, J = 6.48 Hz, 2 H), 4.67 (d, J = 6.60 Hz, 2 H), 4.25 (td, J = 7.73, 7.73, 2.87 Hz, 1 H), 3.85 (m, 1 H), 2.87 (dddd, J = 12.06, 9.06, 6.45, 3.00 Hz, 1 H), 2.67 (m, 1 H), 2.21 (m, 1 H), 1.88 (br d, J = 12.35 Hz, 1 H), 1.69 (br d, J = 10.51 Hz, 3 H), 1.49 (br s, 1 H), 1.29 (m, 2 H), 1.02 (dq, J = 12.65, 12.49, 12.49, 12.49 Hz, 2 H). LC/MS: m/z [M + H]⁺ 465.2, Rt (I = 220 nm): 2.27 min (LC/MS method A), calc. m/z: 465.22. |
| 170 | 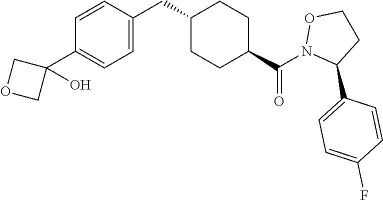 [SMILES: O=C([C@H]1CC[C@H](Cc2ccc(C3(O)COC3)cc2)CC1)N1OCC[C@H]1c1ccc(F)cc1] | 1H NMR (400.23 MHz, DMSO-d6) d ppm 7.49 (d, J = 8.07 Hz, 2 H), 7.29 (dd, J = 8.50, 5.56 Hz, 2 H), 7.17 (dd, J = 8.50, 4.22 Hz, 4 H), 7.13 (m, 1 H), 6.22 (s, 1 H), 5.29 (br t, J = 7.34, 7.34 Hz, 1 H), 4.74 (d, J = 6.48 Hz, 2 H), 4.67 (d, J = 6.48 Hz, 2 H), 4.23 (td, J = 7.70, 7.70, 2.93 Hz, 1 H), 3.82 (m, 1 H), 2.84 (dddd, J = 12.03, 9.03, 6.33, 3.06 Hz, 1 H), 2.66 (br d, J = 9.54 Hz, 1 H), 2.15 (m, 1 H), 1.84 (br d, J = 12.47 Hz, 1 H), 1.68 (br d, J = 12.23 Hz, 3 H), 1.48 (br s, 1 H), 1.28 (m, 2 H), 1.02 (m, 2 H). LC/MS: m/z [M + H]⁺ 440.2, Rt (I = 220 nm): 2.31 min (LC/MS method A), calc. m/z: 440.22. |
| 171 | 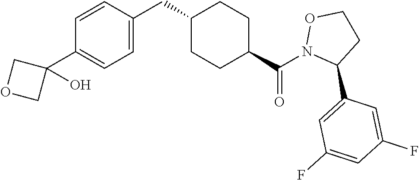 [SMILES: O=C([C@H]1CC[C@H](Cc2ccc(C3(O)COC3)cc2)CC1)N1OCC[C@H]1c1cc(F)cc(F)c1] | ¹H NMR (400 MHz, DMSO-d₆): δ ppm 7.49 (d, J = 8.07 Hz, 2 H), 7.15 (m, 3 H), 6.96 (br d, J = 6.60 Hz, 2 H), 6.22 (s, 1 H), 5.31 (m, 1 H), 4.74 (d, J = 6.48 Hz, 2 H), 4.67 (d, J = 6.48 Hz, 2 H), 4.23 (td, J = 7.73, 7.73, 3.00 Hz, 1 H), 3.83 (m, 1 H), 2.85 (m, 1 H), 2.67 (br s, 1 H), 2.17 (m, 1 H), 1.87 (br d, J = 12.72 Hz, 1 H), 1.68 (br d, J = 5.50 Hz, 3 H), 1.49 (br s, 1 H), 1.29 (m, 2 H), 1.02 (m, 2 H). LC/MS: m/z [M + H]⁺ 458.2, Rt (I = 220 nm): 2.4 min (LC/MS method A), calc. m/z: 458.2. |
| 172 | 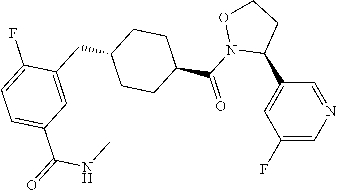 [SMILES: CNC(=O)c1ccc(F)c(C[C@H]2CC[C@H](C(=O)N3OCC[C@H]3c3cncc(F)c3)CC2)c1] | 1H NMR (400.23 MHz, DMSO-d6) d ppm 8.48 (d, J = 2.69 Hz, 1 H), 8.38 (s, 2 H), 7.72 (m, 2 H), 7.57 (br d, J = 9.78 Hz, 1 H), 7.21 (t, J = 9.11, 9.11 Hz, 1 H), 5.38 (m, 1 H), 4.27 (td, J = 7.64, 7.64, 2.93 Hz, 1 H), 3.87 (m, 4 H), 2.88 (dddd, J = 12.03, 9.09, 6.45, 3.00 Hz, 1 H), 2.77 (d, J = 4.52 Hz, 3 H), 2.67 (br s, 1 H), 2.55 (br s, 1 H), 2.26 (m, 1 H), 1.87 (br d, J = 12.72 Hz, 1 H), 1.69 (br s, 3 H), 1.53 (br s, 1 H), 1.29 (m, 2 H), 1.06 (m, 2 H). LC/MS: m/z [M + H]⁺ 444.2, Rt (I = 220 nm): 2 min (LC/MS method A), calc. m/z: 444.21. |
| 173 | 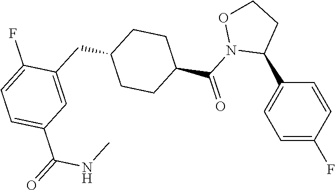 [SMILES: CNC(=O)c1ccc(F)c(C[C@H]2CC[C@H](C(=O)N3OCC[C@H]3c3ccc(F)cc3)CC2)c1] | 1H NMR (400.23 MHz, DMSO-d6) d ppm 8.38 (br d, J = 4.52 Hz, 1 H), 7.72 (m, 2 H), 7.22 (m, 5 H), 5.29 (br t, J = 7.40, 7.40 Hz, 1 H), 4.24 (m, 1 H), 3.83 (m, 1 H), 2.84 (m, 1 H), 2.77 (d, J = 4.52 Hz, 3 H), 2.67 (br s, 1 H), 2.16 (m, 1 H), 1.84 (br d, J = 12.47 Hz, 1 H), 1.66 (br s, 3 H), 1.53 (br s, 1 H), 1.29 (q, J = 12.76, 12.76, 12.76 Hz, 2 H), 1.08 (m, 2 H). LC/MS: m/z [M + H]⁺ 443.2, Rt (I = 220 nm): 2.31 min (LC/MS method A), calc. m/z: 443.22. |
| 174 | 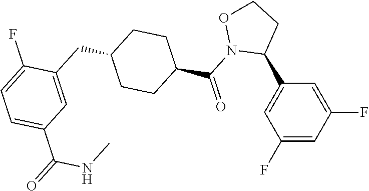 [SMILES: CNC(=O)c1ccc(F)c(C[C@H]2CC[C@H](C(=O)N3OCC[C@H]3c3cc(F)cc(F)c3)CC2)c1] | ¹H NMR (400 MHz, DMSO-d₆): δ ppm 8.39 (br d, J = 4.40 Hz, 1 H), 7.72 (m, 2 H), 7.21 (t, J = 9.11, 9.11 Hz, 1 H), 7.12 (t, J = 8.89, 8.89 Hz, 1 H), 6.96 (br d, J = 6.60 Hz, 2 H), 5.31 (m, 1 H), 4.23 (td, J = 7.61, 7.61, 2.87 Hz, 1 H), 3.84 (m, 1 H), 2.85 (m, 1 H), 2.77 (d, J = 4.52 Hz, 3 H), 2.67 (br s, 1 H), 2.55 (m, 1 H), 2.17 (m, 1 H), 1.88 (br d, J = 13.08 Hz, 1 H), 1.69 (br d, J = 9.90 Hz, 3 H), 1.54 (br s, 1 H), 1.30 (m, 2 H), 1.07 (m, 2 H). LC/MS: m/z [M + H]⁺ 461.2, Rt (I = 220 nm): 2.4 min (LC/MS method A), calc. m/z: 461.21. |
| 175 | 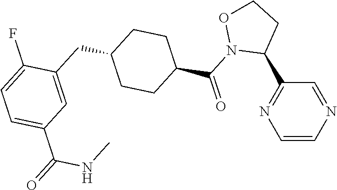 [SMILES: CNC(=O)c1ccc(F)c(C[C@H]2CC[C@H](C(=O)N3OCC[C@H]3c3cnccn3)CC2)c1] | ¹H NMR (400 MHz, DMSO-d₆): δ ppm 8.58 (m, 3 H), 8.38 (br d, J = 4.52 Hz, 1 H), 7.72 (m, 2 H), 7.21 (t, J = 9.05, 9.05 Hz, 1 H), 5.39 (dd, J = 8.68, 6.24 Hz, 1 H), 4.30 (td, J = 7.61, 7.61, 3.36 Hz, 1 H), 3.93 (m, 1 H), 2.81 (m, 1 H), 2.77 (d, J = 4.40 Hz, 2 H), 2.66 (m, 1 H), 1.83 (br d, J = 12.59 Hz, 1 H), 1.67 (br d, J = 13.57 Hz, 3 H), 1.53 (br s, 1 H), 1.28 (m, 2 H), 1.06 (m, 2 H). LC/MS: m/z [M + H]⁺ 427.2, Rt (I = 220 nm): 1.83 min (LC/MS method A), calc. m/z: 427.22. |
| 176 | 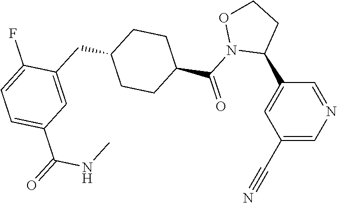 [SMILES: CNC(=O)c1ccc(F)c(C[C@H]2CC[C@H](C(=O)N3OCC[C@H]3c3cncc(C#N)c3)CC2)c1] | 1H NMR (400.23 MHz, DMSO-d6) d ppm 8.93 (d, J = 1.96 Hz, 1 H), 8.76 (d, J = 2.08 Hz, 1 H), 8.38 (br d, J = 4.40 Hz, 1 H), 8.18 (s, 1 H), 7.72 (m, 2 H), 7.21 (t, J = 9.11, 9.11 Hz, 1 H), 5.39 (m, 1 H), 4.28 (td, J = 7.64, 7.64, 2.81 Hz, 1 H), 3.89 (m, 1 H), 2.89 (m, 1 H), 2.77 (d, J = 4.40 Hz, 4 H), 2.68 (m, 1 H), 2.55 (br s, 1 H), 2.29 (m, 1 H), 1.88 (br d, J = 12.59 Hz, 1 H), 1.68 (br d, J = 12.35 Hz, 3 H), 1.53 (br s, 1 H), 1.29 (m, 2 H), 1.06 (br t, J = 11.55, 11.55 Hz, 2 H). LC/MS: m/z [M + H]⁺ 451.2, Rt (I = 220 nm): 1.96 min (LC/MS method A), calc. m/z: 451.22. |
| 177 | 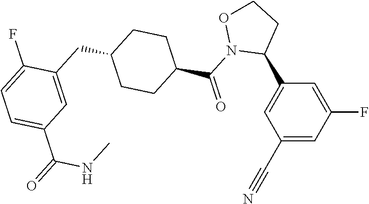 [SMILES: CNC(=O)c1ccc(F)c(C[C@H]2CC[C@H](C(=O)N3OCC[C@H]3c3cc(F)cc(C#N)c3)CC2)c1] | ¹H NMR (400 MHz, DMSO-d₆): δ ppm 8.39 (br m, 1 H), 7.73 (m, 3 H), 7.59 (s, 1 H), 7.46 (br d, J = 9.66 Hz, 1 H), 7.21 (t, J = 9.11, 9.11 Hz, 1 H), 5.35 (m, 1 H), 4.25 (td, J = 7.73, 7.73, 2.87 Hz, 1 H), 3.86 (m, 1 H), 2.87 (m, 1 H), 2.77 (d, J = 4.52 Hz, 3 H), 2.67 (br s, 1 H), 2.55 (br s, 1 H), 2.21 (m, 1 H), 1.89 (br d, J = 13.57 Hz, 1 H), 1.68 (br s, 3 H), 1.54 (br s, 1 H), 1.30 (m, 2 H), 1.06 (m, 2 H). LC/MS: m/z [M + H]⁺ 468.2, Rt (I = 220 nm): 2.27 min (LC/MS method A), calc. m/z: 468.21. |
| 178 | 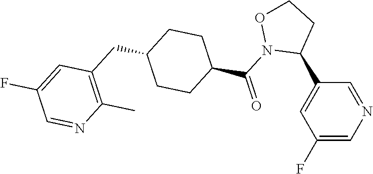 [SMILES: Cc1ncc(F)cc1C[C@H]1CC[C@H](C(=O)N2OCC[C@H]2c2cncc(F)c2)CC1] | ¹H NMR (400 MHz, DMSO-d₆): δ ppm 8.49 (d, J = 2.75 Hz, 1 H), 8.39 (s, 1 H), 8.37 (d, J = 2.75 Hz, 1 H), 7.62 (dd, J = 9.54, 2.38 Hz, 1 H), 7.58 (dt, J = 9.77, 2.18, 2.18 Hz, 1 H), 5.39 (m, 1 H), 4.28 (td, J = 7.79, 7.79, 3.12 Hz, 1 H), 3.88 (ddd, J = 9.26, 8.07, 6.69 Hz, 1 H), 2.89 (m, 1 H), 2.69 (br s, 1 H), 2.38 (m, 1 H), 1.87 (m, 1 H), 1.71 (m, 3 H), 1.56 (m, 1 H), 1.30 (m, 2 H), 1.06 (m, 2 H). LC/MS: m/z [M + H]⁺ 402.2, Rt (I = 220 nm): 1.87 min (LC/MS method A), calc. m/z: 402.20. |
| 179 | 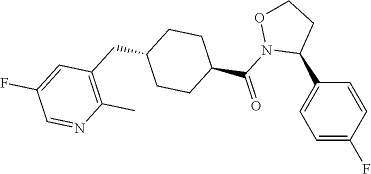 [SMILES: Cc1ncc(F)cc1C[C@H]1CC[C@H](C(=O)N2OCC[C@H]2c2ccc(F)cc2)CC1] | ¹H NMR (400 MHz, DMSO-d₆): δ ppm 8.34 (d, J = 2.75 Hz, 1 H), 7.57 (dd, J = 9.63, 2.11 Hz, 1 H), 7.29 (t, J = 6.61, 6.61 Hz, 2 H), 7.15 (t, J = 8.22, 8.22 Hz, 2 H), 5.29 (br t, J = 7.24, 7.24 Hz, 1 H), 4.24 (td, J = 7.66, 7.66, 2.84 Hz, 1 H), 3.83 (m, 1 H), 2.84 (m, 1 H), 2.68 (br s, 1 H), 2.16 (m, 1 H), 1.84 (br d, J = 12.65 Hz, 1 H), 1.67 (br d, J = 8.80 Hz, 3 H), 1.55 (m, 1 H), 1.31 (m, 2 H), 1.11 (m, 2 H). LC/MS: m/z [M + H]⁺ 401.2, Rt (I = 220 nm): 2.26 min (LC/MS method A), calc. m/z: 401.20. |
| 180 | 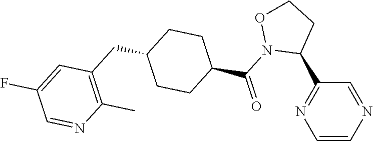 [SMILES: Cc1ncc(F)cc1C[C@H]1CC[C@H](C(=O)N2OCC[C@H]2c2cnccn2)CC1] | ¹H NMR (600 MHz, DMSO-d₆): δ ppm 8.61 (dd, J = 2.38, 1.47 Hz, 1 H), 8.57 (dd, J = 4.68, 1.93 Hz, 2 H), 8.32 (d, J = 2.94 Hz, 1 H), 7.55 (d, J = 9.23 Hz, 1 H), 5.40 (dd, J = 8.80, 6.05 Hz, 1 H), 4.30 (td, J = 7.66, 7.66, 3.39 Hz, 1 H), 3.94 (m, 1 H), 2.82 (m, 1 H), 2.68 (br s, 1 H), 2.55 (m, 2 H), 2.45 (m, 3 H), 1.83 (br d, J = 12.47 Hz, 1 H), 1.71 (br s, 1 H), 1.66 (br d, J = 11.19 Hz, 3 H), 1.54 (m, 1 H), 1.30 (m, 2 H), 1.10 (m, 2 H). LC/MS: m/z [M + H]⁺ 385.2, Rt (I = 220 nm): 1.62 min (LC/MS method A), calc. m/z: 385.20. |
| 181 | 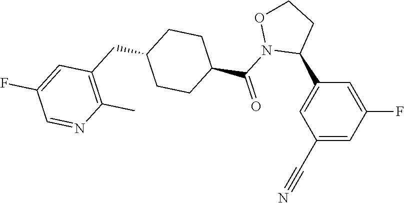 [SMILES: Cc1ncc(F)cc1C[C@H]1CC[C@H](C(=O)N2OCC[C@H]2c2cc(F)cc(C#N)c2)CC1] | 1H NMR (600 MHz, DMSO-d₆): δ ppm 8.33 (d, J = 2.75 Hz, 1 H), 7.76 (d, J = 8.34 Hz, 1 H), 7.59 (s, 1 H), 7.56 (m, 1 H), 7.46 (br d, J = 9.54 Hz, 1 H), 5.36 (m, 1 H), 4.26 (m, 1 H), 3.86 (m, 1 H), 2.88 (m, 1 H), 2.70 (br s, 1 H), 2.39 (m, 1 H), 2.21 (m, 1 H), 1.89 (m, 1 H), 1.68 (m, 3 H), 1.56 (m, 1 H), 1.37 (s, 1 H), 1.31 (m, 2 H), 1.11 (m, 2 H). LC/MS: m/z [M + H]⁺ 426.2, Rt (I = 220 nm): 2.22 min (LC/MS method A), calc. m/z: 426.20. |
| 182 | 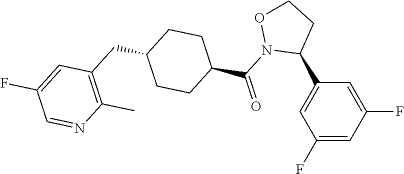 [SMILES: Cc1ncc(F)cc1C[C@H]1CC[C@H](C(=O)N2OCC[C@H]2c2cc(F)cc(F)c2)CC1] | ¹H NMR (600 MHz, DMSO-d₆): δ ppm 8.32 (d, J = 2.75 Hz, 1 H), 7.55 (br d, J = 9.35 Hz, 1 H), 7.12 (tt, J = 9.26, 9.26, 2.29, 2.29 Hz, 1 H), 6.96 (br d, J = 6.42 Hz, 2 H), 5.32 (m, 1 H), 4.24 (td, J = 7.75, 7.75, 3.03 Hz, 1 H), 3.84 (m, 1 H), 2.86 (m, 1 H), 2.69 (br s, 1 H), 2.39 (s, 3 H), 2.18 (m, 1 H), 1.88 (br d, J = 12.65 Hz, 1H), 1.67 (m, 3 H), 1.56 (m, 1 H), 1.31 (m, 2 H), 1.11 (m, 2 H). LC/MS: m/z [M + H]⁺ 419.2, Rt (I = 220 nm): 2.37 min (LC/MS method A), calc. m/z: 419.20. |
| 183 | 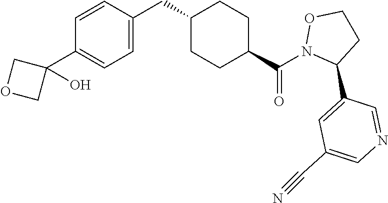 [SMILES: N#Cc1cncc([C@@H]2CCON2C(=O)[C@H]2CC[C@H](Cc3ccc(C4(O)COC4)cc3)CC2)c1] | 1H NMR (400.23 MHz, DMSO-d6) d ppm 8.93 (d, J = 1.83 Hz, 1 H), 8.76 (d, J = 2.08 Hz, 1 H), 8.18 (s, 1 H), 7.49 (m(para), J = 8.07 Hz, 2 H), 7.71 (m(para), J = 8.07 Hz, 2 H), 6.22 (br s, 1 H), 5.39 (m, 1 H), 4.74 (d, J = 6.48 Hz, 2 H), 4.67 (d, J = 6.48 Hz, 2 H), 4.28 (m, 1 H), 3.89 (m, 1 H), 2.88 (m, 1 H), 2.66 (m, 1 H), 2.29 (m, 1 H), 1.86 (br d, J = 12.84 Hz, 1 H), 1.69 (br d, J = 10.76 Hz, 3 H), 1.49 (br s, 1 H), 1.28 (q, J = 11.82, 11.82, 11.82 Hz, 2 H), 1.02 (m, 2 H). LC/MS: m/z [M + H]⁺ 448.2, Rt (I = 220 nm): 1.94 min (LC/MS method A), calc. m/z: 448.22. |
| 184 | 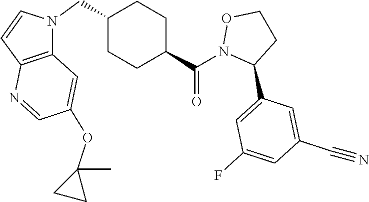 [SMILES: CC1(Oc2cnc3ccn(C[C@H]4CC[C@H](C(=O)N5OCC[C@H]5c5cc(F)cc(C#N)c5)CC4)c3c2)CC1] | 1H NMR (400 MHz, DMSO-d6): δ ppm 8.09 (d, J = 2.4 Hz, 1H), 7.76 (br d, J = 7.7 Hz, 1H), 7.58 (s, 1H), 7.48 (t, J = 2.9 Hz, 2H), 7.45 (br d, J = 9.5 Hz, 1H), 6.47 (d, J = 3.1 Hz, 1H), 5.37-5.31 (m, 1H), 4.24 (dt, J = 2.7, 7.6 Hz, 1H), 4.02 (br d, J = 7.0 Hz, 2H), 3.88-3.81 (m, 1H), 2.86 (dqd, J = 2.8, 6.2, 9.1 Hz, 1H), 2.70- 2.63 (m, 1H), 2.23-2.16 (m, 1H), 1.89 (br d, J = 10.6 Hz, 1H), 1.79-1.73 (m, 1H), 1.72-1.66 (m, 1H), 1.63- 1.55 (m, 2H), 1.53 (s, 3H), 1.30-1.23 (m, 2H), 1.16- 1.05 (m, 2H), 0.98-0.94 (m, 2H), 0.81-0.77 (m, 2H). LC/MS: m/z [M + H]⁺ 503.3, Rt (I = 220 nm): 0.584 min (LC/MS method B), calc. m/z: 503.25. |
| 185 | 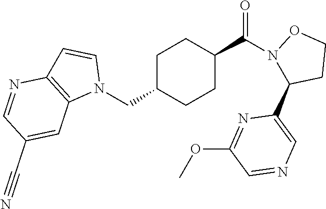 [SMILES: COc1cncc([C@@H]2CCON2C(=O)[C@H]2CC[C@H](Cn3ccc4ncc(C#N)cc43)CC2)n1] | 1H NMR (400.23 MHz, DMSO-d6) d ppm 8.66 (s, 1 H), 8.65 (s, 1 H), 8.20 (s, 1 H), 8.10 (s, 1 H), 8.00 (d, J = 3.18 Hz, 1 H), 6.74 (d, J = 2.81 Hz, 1 H), 5.30 (dd, J = 8.93, 5.50 Hz, 1 H), 4.28 (td, J = 7.55, 7.55, 4.22 Hz, 1 H), 4.14 (d, J = 7.21 Hz, 2 H), 3.94 (m, 2 H), 3.89 (s, 3 H), 2.78 (m, 1 H), 2.65 (m, 1 H), 2.43 (br d, J = 5.14 Hz, 1 H), 1.85 (br d, J = 11.74 Hz, 2 H), 1.70 (br d, J = 11.86 Hz, 1 H), 1.52 (br s, 2 H), 1.27 (m, 2 H), 1.10 (m, 2 H). LC/MS: m/z [M + H]⁺ 447.2, Rt (I = 220 nm): 1.9 min (LC/MS method A), calc. m/z: 447.22. |
| 186 | 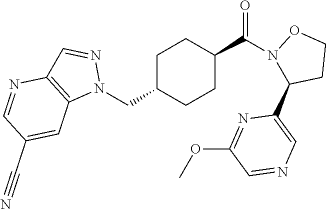 [SMILES: COc1cncc([C@@H]2CCON2C(=O)[C@H]2CC[C@H](Cn3ncc4ncc(C#N)cc43)CC2)n1] | 1H NMR (400.23 MHz, DMSO-d6) d ppm 9.01 (s, 1 H), 8.85 (d, J = 1.59 Hz, 1 H), 8.50 (s, 1 H), 8.20 (s, 1 H), 8.10 (s, 1 H), 7.60 (m, 1 H), 5.30 (br dd, J = 8.50, 5.69 Hz, 1 H), 4.37 (br d, J = 6.97 Hz, 2 H), 4.28 (m, 3 H), 4.03 (br s, 6 H), 3.94 (m, 3 H), 3.89 (s, 5 H), 3.70 (br s, 1 H), 2.78 (m, 1 H), 2.67 (m, 1 H), 2.42 (m, 1 H), 1.92 (br s, 1 H), 1.85 (br d, J = 12.84 Hz, 1 H), 1.70 (br d, J = 10.51 Hz, 1 H), 1.54 (br s, 2 H), 1.28 (m, 2 H), 1.12 (m, 2 H). LC/MS: m/z [M + H]⁺ 448.2, Rt (I = 220 nm): 1.91 min (LC/MS method A), calc. m/z: 448.21. |
| 187 | 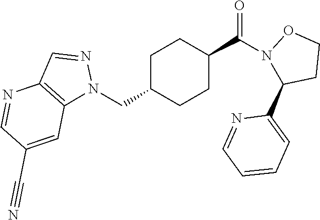 [SMILES: N#Cc1cnc2cnn(C[C@H]3CC[C@H](C(=O)N4OCC[C@H]4c4ccccn4)CC3)c2c1] | 1H NMR (400.23 MHz, DMSO-d6) d ppm 9.01 (s, 1 H), 8.85 (d, J = 1.47 Hz, 1 H), 8.53 (br d, J = 4.65 Hz, 1 H), 8.50 (s, 1 H), 7.80 (m, 1 H), 7.31 (m, 2 H), 5.33 (dd, J = 8.68, 6.11 Hz, 1 H), 4.38 (d, J = 6.97 Hz, 2 H), 4.23 (dt, J = 7.43, 3.68, 3.68 Hz, 1 H), 3.91 (br s, 4 H), 3.86 (br dd, J = 14.67, 7.34 Hz, 22 H), 2.79 (m, 1 H), 2.67 (br s, 1 H), 2.42 (m, 1 H), 1.93 (br s, 1 H), 1.85 (br d, J = 11.98 Hz, 1 H), 1.68 (br s, 1 H), 1.55 (m, 2 H), 1.29 (q, J = 12.76, 12.76, 12.76 Hz, 2 H), 1.13 (m, 2 H). LC/MS: m/z [M + H]⁺ 417.2, Rt (I = 220 nm): 1.68 min (LC/MS method A), calc. m/z: 417.20. |
| 188 | 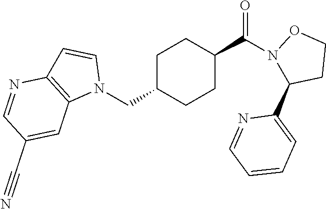 [SMILES: N#Cc1cnc2ccn(C[C@H]3CC[C@H](C(=O)N4OCC[C@H]4c4ccccn4)CC3)c2c1] | 1H NMR (400.23 MHz, DMSO-d6) d ppm 8.67 (t, J = 2.32, 2.32 Hz, 2 H), 8.54 (d, J = 4.40 Hz, 1 H), 8.01 (d, J = 3.18 Hz, 1 H), 7.82 (t, J = 7.83, 7.83 Hz, 1 H), 7.33 (m, 2 H), 6.75 (d, J = 3.06 Hz, 1 H), 5.34 (dd, J = 8.80, 5.99 Hz, 1 H), 4.24 (td, J = 7.67, 7.67, 3.48 Hz, 4 H), 4.14 (br d, J = 7.09 Hz, 3 H), 3.88 (m, 1 H), 2.80 (m, 1 H), 2.66 (br d, J = 12.23 Hz, 1 H), 2.42 (m, 1 H), 1.85 (br d, J = 11.62 Hz, 2 H), 1.69 (br s, 1 H), 1.53 (m, 2 H), 1.29 (td, J = 12.59, 12.59, 9.05 Hz, 2 H), 1.09 (m, 2H). LC/MS: m/z [M + H]⁺ 416.2, Rt (I = 220 nm): 1.69 min (LC/MS method A), calc. m/z: 416.21. |
| 189 | 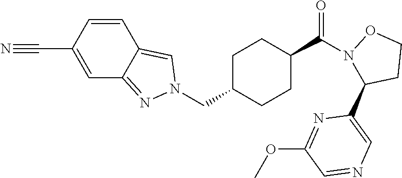 [SMILES: COc1cncc([C@@H]2CCON2C(=O)[C@H]2CC[C@H](Cn3cc4ccc(C#N)cc4n3)CC2)n1] | 1H NMR (400.23 MHz, DMSO-d6) d ppm 8.55 (s, 1 H), 8.31 (s, 1 H), 8.20 (s, 1 H), 8.11 (s, 1 H), 7.92 (d, J = 8.56 Hz, 1 H), 7.28 (dd, J = 8.56, 1.22 Hz, 1 H), 5.31 (dd, J = 8.80, 5.75 Hz, 1 H), 4.37 (d, J = 6.97 Hz, 2H), 4.28 (td, J = 7.61, 7.61, 4.22 Hz, 1 H), 3.94 (m, 4 H), 3.51 (br s, 1 H), 2.77 (m, 1 H), 2.66 (m, 1 H), 2.43 (m, 1 H), 1.98 (br s, 1 H), 1.87 (br d, J = 12.84 Hz, 1 H), 1.71 (br d, J = 13.57 Hz, 1 H), 1.57 (br d, J = 12.47 Hz, 2 H), 1.29 (m, 2 H), 1.11 (m, 2H). LC/MS: m/z [M + H]⁺ 447.2, Rt (I = 220 nm): 2.04 min (LC/MS method A), calc. m/z: 447.22. |
| 190 | 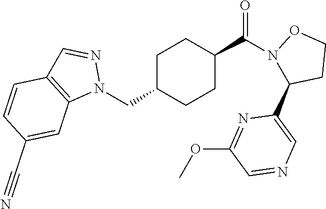 [SMILES: COc1cncc([C@@H]2CCON2C(=O)[C@H]2CC[C@H](Cn3ncc4ccc(C#N)cc43)CC2)n1] | 1H NMR (400.23 MHz, DMSO-d6) d ppm 8.46 (s, 1 H), 8.22 (m, 2 H), 8.10 (s, 1 H), 7.97 (d, J = 8.31 Hz, 1 H), 7.45 (dd, J = 8.25, 1.04 Hz, 1 H), 5.31 (m, 1 H), 4.31 (m, 6 H), 4.11 (m, 6 H), 3.94 (m, 2 H), 3.89 (s, 4 H), 3.51 (d, J = 6.11 Hz, 1 H), 3.23 (q, J = 7.09, 7.09, 7.09 Hz, 1 H), 2.78 (m, 1 H), 2.65 (m, 1 H), 2.42 (m, 1 H), 1.91 (dt, J = 7.37, 3.59, 3.59 Hz, 1 H), 1.85 (br d, J = 12.47 Hz, 1 H), 1.69 (br d, J = 11.86 Hz, 1 H), 1.54 (m, 2 H), 1.27 (m, 2 H), 1.09 (m, 3 H). LC/MS: m/z [M + H]⁺ 447.2, Rt (I = 220 nm): 2.16 min (LC/MS method A), calc. m/z: 447.22. |
| 191 | 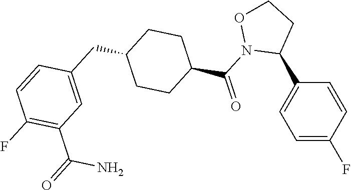 [SMILES: NC(=O)c1cc(C[C@H]2CC[C@H](C(=O)N3OCC[C@H]3c3ccc(F)cc3)CC2)ccc1F] | 1H NMR (400.23 MHz, DMSO-d6) d ppm 7.62 (br s, 1 H), 7.59 (br s, 1 H), 7.42 (dd, J = 7.03, 2.14 Hz, 1 H), 7.29 (m, 3 H), 7.15 (m, 3 H), 5.29 (br t, J = 7.34, 7.34 Hz, 1 H), 4.23 (td, J = 7.61, 7.61, 2.87 Hz, 1 H), 3.83 (m, 1 H), 2.84 (m, 1 H), 2.66 (br d, J = 10.03 Hz, 1 H), 2.16 (m, 1 H), 1.84 (br d, J = 12.23 Hz, 1 H), 1.67 (br d, J = 12.47 Hz, 3 H), 1.47 (br s, 1 H), 1.28 (m, 2 H), 1.01 (m, 2 H). LC/MS: m/z [M + H]⁺ 429.2, Rt (I = 220 nm): 2.22 min (LC/MS method A), calc. m/z: 429.20. |
| 192 | 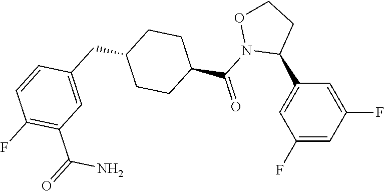 [SMILES: NC(=O)c1cc(C[C@H]2CC[C@H](C(=O)N3OCC[C@H]3c3cc(F)cc(F)c3)CC2)ccc1F] | ¹H NMR (400 MHz, DMSO-d₆): δ ppm 7.62 (br s, 1 H), 7.58 (br s, 1 H), 7.43 (dd, J = 7.09, 2.20 Hz, 1 H), 7.29 (m, 1 H), 7.14 (m, 2 H), 6.96 (br d, J = 6.48 Hz, 2 H), 5.31 (dd, J = 8.25, 6.54 Hz, 1 H), 4.23 (td, J = 7.73, 7.73, 2.75 Hz, 1 H), 3.83 (m, 1 H), 2.85 (m, 1 H), 2.67 (br s, 1 H), 2.17 (m, 1 H), 1.87 (br d, J = 12.23 Hz, 1 H), 1.68 (m, 3 H), 1.48 (br s, 1 H), 1.29 (m, 2 H), 1.02 (m, 2 H). LC/MS: m/z [M + H]⁺ 447.2, Rt (I = 220 nm): 2.32 min (LC/MS method A), calc. m/z: 447.19. |
| 193 | 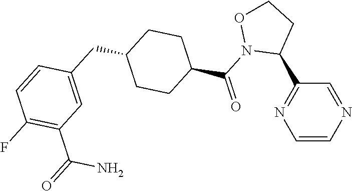 [SMILES: NC(=O)c1cc(C[C@H]2CC[C@H](C(=O)N3OCC[C@H]3c3cnccn3)CC2)ccc1F] | 1H NMR (400.23 MHz, DMSO-d6) d ppm 8.58 (m, 3 H), 7.62 (br s, 1 H), 7.58 (br s, 1 H), 7.42 (dd, J = 6.97, 2.08 Hz, 1 H), 7.29 (m, 1 H), 7.16 (dd, J = 10.64, 8.44 Hz, 1 H), 5.39 (dd, J = 8.93, 6.24 Hz, 1 H), 4.29 (m, 1 H), 3.93 (m, 1 H), 2.81 (br dd, J = 6.11, 2.81 Hz, 1 H), 2.66 (br d, J = 8.68 Hz, 1 H), 1.82 (br d, J = 11.86 Hz, 1 H), 1.67 (br d, J = 12.23 Hz, 3 H), 1.28 (m, 2 H), 1.01 (m, 2 H). LC/MS: m/z [M + H]⁺ 413.2, Rt (I = 220 nm): 1.72 min (LC/MS method A), calc. m/z: 413.20. |
| 194 | 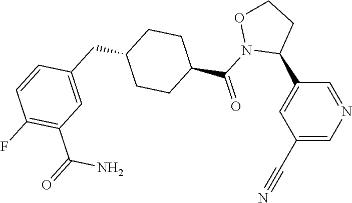 [SMILES: N#Cc1cncc([C@@H]2CCON2C(=O)[C@H]2CC[C@H](Cc3ccc(F)c(C(N)=O)c3)CC2)c1] | 1H NMR (400.23 MHz, DMSO-d6) d ppm 8.93 (d, J = 1.83 Hz, 1 H), 8.76 (d, J = 2.08 Hz, 1 H), 8.18 (t, J = 1.90, 1.90 Hz, 1 H), 7.62 (br s, 1 H), 7.58 (br s, 1 H), 7.42 (dd, J = 7.09, 2.20 Hz, 1 H), 7.29 (ddd, J = 8.01, 5.20, 2.32 Hz, 1 H), 7.16 (dd, J = 10.51, 8.44 Hz, 1 H), 5.39 (m, 1 H), 4.28 (td, J = 7.70, 7.70, 2.93 Hz, 1 H), 3.89 (m, 2 H, under water peak), 2.88 (dddd, J = 12.17, 9.14, 6.39, 3.00 Hz, 1 H), 2.67 (m, 1 H), 2.29 (m, 1 H), 1.87 (br d, J = 12.0 Hz, 1 H), 1.67 (br d, J = 12.35 Hz, 3 H), 1.48 (br s, 1H), 1.28 (m, 2H), 1.11 (m, 2H). LC/MS: m/z [M + H]⁺ 437.2, Rt (I = 220 nm): 1.87 min (LC/MS method A), calc. m/z: 437.20. |
| 195 | 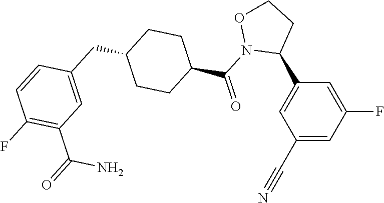 [SMILES: N#Cc1cc(F)cc([C@@H]2CCON2C(=O)[C@H]2CC[C@H](Cc3ccc(F)c(C(N)=O)c3)CC2)c1] | ¹H NMR (400 MHz, DMSO-d₆): δ ppm 7.76 (br d, J = 8.44 Hz, 1 H), 7.62 (m, 3 H), 7.46 (m, 2 H), 7.29 (m, 1 H), 7.16 (dd, J = 10.51, 8.44 Hz, 1 H), 5.36 (m, 1 H), 4.25 (m, 1 H), 3.86 (m, 1 H), 3.58 (s, 2 H), 2.87 (m, 1 H), 2.66 (m, 1 H), 2.21 (m, 1 H), 1.88 (br d, J = 12.96 Hz, 1 H), 1.68 (br s, 3 H), 1.48 (br s, 1 H), 1.29 (m, 2 H), 1.02 (m, 2 H). LC/MS: m/z [M + H]⁺ 454.2, Rt (I = 220 nm): 2.18 min (LC/MS method A), calc. m/z: 454.19. |
| 196 | 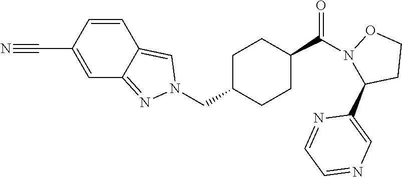 [SMILES: N#Cc1ccc2cn(C[C@H]3CC[C@H](C(=O)N4OCC[C@H]4c4cnccn4)CC3)nc2c1] | LC/MS: m/z [M + H]⁺ 417.2, Rt (I = 220 nm): 1.8 min (LC/MS method A), calc. m/z: 417.20. |
| 197 | 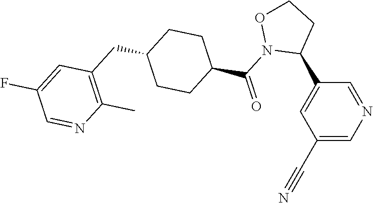 [SMILES: Cc1ncc(F)cc1C[C@H]1CC[C@H](C(=O)N2OCC[C@H]2c2cncc(C#N)c2)CC1] | ¹H NMR (400 MHz, DMSO-d₆): δ ppm 8.93 (d, J = 1.83 Hz, 1 H), 8.76 (d, J = 2.08 Hz, 1 H), 8.31 (d, J = 2.69 Hz, 1 H), 8.18 (s, 1 H), 7.54 (d, J = 9.54 Hz, 1 H), 5.40 (m, 1 H), 4.29 (td, J = 7.61, 7.61, 3.00 Hz, 1 H), 3.89 (m, 1 H), 2.89 (m, 1 H), 2.68 (br d, J = 10.88 Hz, 1 H), 2.30 (m, 1 H), 1.87 (br d, J = 13.33 Hz, 1 H), 1.68 (m, 3 H), 1.55 (br s, 1 H), 1.29 (m, 2 H), 1.09 (m, 2 H). LC/MS: m/z [M + H]⁺ 409.2, Rt (I = 220 nm): 1.82 min (LC/MS method A), calc. m/z: 409.20. |
| 198 | 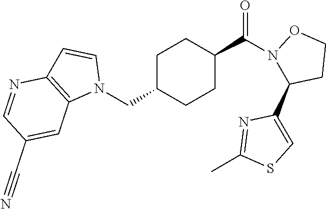 [SMILES: Cc1nc([C@@H]2CCON2C(=O)[C@H]2CC[C@H](Cn3ccc4ncc(C#N)cc43)CC2)cs1] | 1H NMR (400.23 MHz, DMSO-d6) d ppm 8.66 (d, J = 4.92 Hz, 1 H), 8.00 (d, J = 3.18 Hz, 1 H), 7.17 (s, 1 H), 6.74 (d, J = 2.81 Hz, 1 H), 5.34 (m, 1 H), 4.22 (td, J = 7.67, 7.67, 3.73 Hz, 1 H), 4.13 (d, J = 7.09 Hz, 1 H), 3.84 (m, 3 H), 2.69 (m, 1 H), 2.61 (s, 2 H), 2.38 (m, 1 H), 1.81 (br d, J = 11.98 Hz, 1 H), 1.66 (br s, 1 H), 1.51 (br s, 1 H), 1.27 (m, 1 H), 1.09 (m, 1 H). LC/MS: m/z [M + H]⁺ 436.2, Rt (I = 220 nm): 1.84 min (LC/MS method A), calc. m/z: 436.18. |
| 199 | 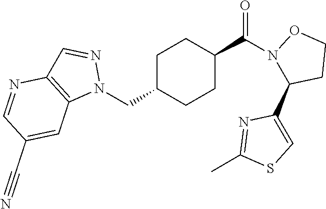 [SMILES: Cc1nc([C@@H]2CCON2C(=O)[C@H]2CC[C@H](Cn3ncc4ncc(C#N)cc43)CC2)cs1] | 1H NMR (400.23 MHz, DMSO-d6) d ppm 9.01 (s, 1 H), 8.85 (d, J = 1.59 Hz, 1 H), 8.50 (s, 1 H), 7.17 (s, 1 H), 5.34 (dd, J = 8.74, 5.69 Hz, 1 H), 4.37 (d, J = 6.97 Hz, 2 H), 4.22 (td, J = 7.61, 7.61, 3.61 Hz, 1 H), 2.68 (m, 1 H), 2.61 (s, 4 H), 2.38 (m, 2 H), 1.92 (br d, J = 3.79 Hz, 1 H), 1.81 (br d, J = 12.72 Hz, 1 H), 1.65 (br s, 1 H), 1.53 (br s, 2 H), 1.27 (m, 2 H), 1.12 (m, 2 H). LC/MS: m/z [M + H]⁺ 437.2, Rt (I = 220 nm): 1.85 min (LC/MS method A), calc. m/z: 437.18. |
| 200 | 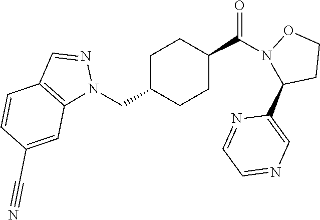 [SMILES: N#Cc1ccc2cnn(C[C@H]3CC[C@H](C(=O)N4OCC[C@H]4c4cnccn4)CC3)c2c1] | 1H NMR (400.23 MHz, DMSO-d6) d ppm 8.57 (m, 3 H), 8.46 (s, 1 H), 8.25 (s, 1 H), 7.97 (d, J = 8.19 Hz, 1 H), 7.45 (d, J = 8.31 Hz, 1 H), 5.39 (m, 1 H), 4.33 (m, 2 H), 3.92 (br d, J = 9.29 Hz, 1 H), 3.51 (br d, J = 6.11 Hz, 2 H), 2.80 (m, 1 H), 2.67 (br d, J = 4.16 Hz, 1 H), 2.65 (br s, 1 H), 1.83 (br s, 3 H), 1.72 (br s, 2 H), 1.51 (br s, 2 H), 1.31 (br s, 1 H), 1.26 (br d, J = 12.59 Hz, 2 H), 1.12 (m, 2 H). LC/MS: m/z [M + H]⁺ 417.2, Rt (I = 220 nm): 1.92 min (LC/MS method A), calc. m/z: 417.20. |
| 201 | 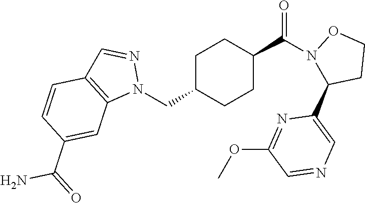 [SMILES: COc1cncc([C@@H]2CCON2C(=O)[C@H]2CC[C@H](Cn3ncc4ccc(C(N)=O)cc43)CC2)n1] | 1H NMR (400.23 MHz, DMSO-d6) d ppm 8.20 (d, J = 4.77 Hz, 3 H), 8.11 (d, J = 5.99 Hz, 2 H), 8.04 (m, 1 H), 7.79 (d, J = 8.31 Hz, 1 H), 7.63 (d, J = 8.56 Hz, 1 H), 7.45 (br s, 1 H), 5.31 (br s, 1 H), 4.30 (m, 3 H), 3.92 (m, 1 H), 3.88 (s, 4 H), 3.51 (br s, 2 H), 3.17 (s, 3 H), 2.67 (br s, 2 H), 1.56 (br s, 3 H), 1.26 (m, 3 H), 1.15 (br d, J = 13.3 Hz, 3 H). LC/MS: m/z [M + H]⁺ 465.2, Rt (I = 220 nm): 1.69 min (LC/MS method A), calc. m/z: 465.23. |
| 202 | 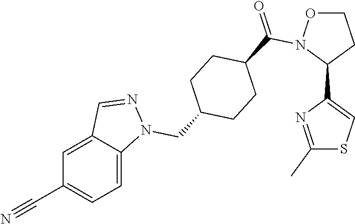 [SMILES: Cc1nc([C@@H]2CCON2C(=O)[C@H]2CC[C@H](Cn3ncc4cc(C#N)ccc43)CC2)cs1] | 1H NMR (400.23 MHz, DMSO-d6) d ppm 8.40 (s, 1 H), 8.27 (s, 1 H), 7.91 (d, J = 8.93 Hz, 1 H), 7.71 (dd, J = 8.80, 1.34 Hz, 1 H), 7.17 (s, 1 H), 5.34 (br dd, J = 8.50, 5.81 Hz, 1 H), 4.33 (d, J = 6.97 Hz, 2 H), 4.21 (td, J = 7.61, 7.61, 3.97 Hz, 1 H), 3.83 (m, 4 H), 2.69 (m, 1 H), 2.61 (s, 4 H), 2.38 (m, 1 H), 1.89 (br s, 1 H), 1.81 (br d, J = 12.23 Hz, 1 H), 1.65 (br s, 1 H), 1.53 (br s, 2 H), 1.26 (m, 2 H), 1.12 (m, 2 H) LC/MS: m/z [M + H]⁺ 436.2, Rt (I = 220 nm): 2.05 min (LC/MS method A), calc. m/z: 436.18. |
| 203 | 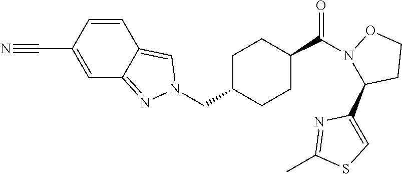 [SMILES: Cc1nc([C@@H]2CCON2C(=O)[C@H]2CC[C@H](Cn3cc4ccc(C#N)cc4n3)CC2)cs1] | 1H NMR (400.23 MHz, DMSO-d6) d ppm 8.55 (s, 1 H), 8.31 (s, 1 H), 7.92 (d, J = 8.31 Hz, 1 H), 7.28 (dd, J = 8.56, 1.22 Hz, 1 H), 7.18 (s, 1 H), 5.34 (m, 1 H), 4.37 (d, J = 6.97 Hz, 2 H), 4.22 (td, J = 7.49, 7.49, 4.10 Hz, 1 H), 3.84 (q, J = 7.74, 7.74, 7.74 Hz, 1 H), 2.68 (m, 2 H), 2.61 (s, 4 H), 2.38 (m, 2 H), 1.97 (br s, 1 H), 1.83 (br d, J = 12.84 Hz, 1 H), 1.67 (br s, 1 H), 1.56 (br d, J = 11.98 Hz, 2 H), 1.29 (m, 2 H), 1.11 (m, 2 H). LC/MS: m/z [M + H]⁺ 436.2, Rt (I = 220 nm): 1.98 min (LC/MS method A), calc. m/z: 436.18. |
| 204 | 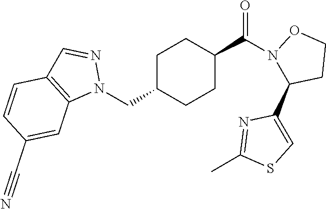 [SMILES: Cc1nc([C@@H]2CCON2C(=O)[C@H]2CC[C@H](Cn3ncc4ccc(C#N)cc43)CC2)cs1] | 1H NMR (400.23 MHz, DMSO-d6) d ppm 7.18 (br d, J = 8.19 Hz, 1 H), 5.36 (m, 1 H), 4.35 (d, J = 7.09 Hz, 1 H), 4.22 (m, 2 H), 4.05 (br s, 6 H), 3.85 (m, 2 H), 3.51 (d, J = 6.11 Hz, 1 H), 2.62 (d, J = 4.89 Hz, 4 H), 2.39 (m, 1 H), 1.90 (br s, 1 H), 1.84 (br d, J = 13.69 Hz, 1 H), 1.70 (br s, 1 H), 1.57 (m, 1 H), 1.31 (m, 2 H), 1.10 (m, 2 H). LC/MS: m/z [M + H]⁺ 436.2, Rt (I = 220 nm): 2.11 min (LC/MS method A), calc. m/z: 436.18. |
| 205 | 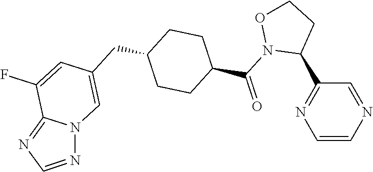 [SMILES: O=C([C@H]1CC[C@H](Cc2cc(F)c3ncnn3c2)CC1)N1OCC[C@H]1c1cnccn1] | 1H NMR (400.23 MHz, DMSO-d6) d ppm 8.71 (s, 1 H), 8.56 (m, 4 H), 7.57 (d, J = 11.25 Hz, 1 H), 5.40 (dd, J = 8.74, 6.17 Hz, 1 H), 4.30 (m, 6 H), 3.93 (m, 1 H), 2.81 (m, 1 H), 2.66 (m, 1 H), 2.58 (d, J = 6.97 Hz, 2 H), 1.83 (br d, J = 12.72 Hz, 1 H), 1.67 (m, 4 H), 1.30 (m, 2 H), 1.04 (m, 2 H). LC/MS: m/z [M + H]⁺ 411.2, Rt (I = 220 nm): 1.66 min (LC/MS method A), calc. m/z: 411.20. |
| 206 | 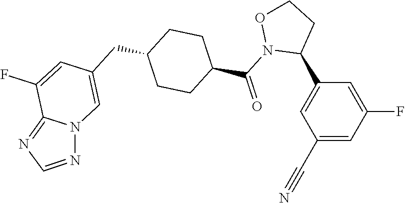 [SMILES: N#Cc1cc(F)cc([C@@H]2CCON2C(=O)[C@H]2CC[C@H](Cc3cc(F)c4ncnn4c3)CC2)c1] | 1H NMR (400.23 MHz, DMSO-d6) d ppm 8.71 (s, 1 H), 8.52 (s, 1 H), 7.76 (br d, J = 8.56 Hz, 1 H), 7.58 (m, 2 H), 7.46 (br d, J = 9.41 Hz, 1 H), 5.36 (dd, J = 8.31, 6.60 Hz, 1 H), 4.25 (td, J = 7.73, 7.73, 2.75 Hz, 1 H), 3.86 (m, 3 H), 2.87 (m, 1 H), 2.68 (br d, J = 1.96 Hz, 1 H), 2.59 (d, J = 6.97 Hz, 2 H), 2.33 (s, 1 H), 2.21 (m, 1 H), 1.89 (br d, J = 11.13 Hz, 1 H), 1.70 (br d, J = 9.78 Hz, 3 H), 1.62 (br s, 1 H), 1.32 (m, 2 H), 1.04 (tdd, J = 12.59, 12.59, 12.47, 12.23 Hz, 2 H). LC/MS: m/z [M + H]⁺ 452.2, Rt (I = 220 nm): 2.14 min (LC/MS method A), calc. m/z: 452.19. |
| 207 | 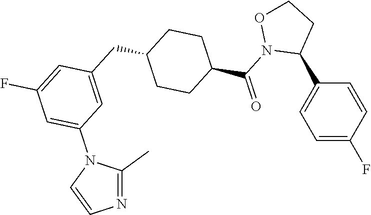 [SMILES: Cc1nccn1-c1cc(F)cc(C[C@H]2CC[C@H](C(=O)N3OCC[C@H]3c3ccc(F)cc3)CC2)c1] | 1H NMR (400.23 MHz, DMSO-d6) d ppm 7.89 (d, J = 2.08 Hz, 1 H), 7.76 (d, J = 2.08 Hz, 1 H), 7.44 (br d, J = 8.93 Hz, 1 H), 7.31 (m, 4 H), 7.16 (t, J = 8.29, 8.29 Hz, 2 H), 5.29 (m, 1 H), 4.24 (td, J = 7.64, 7.64, 2.93 Hz, 1 H), 3.83 (m, 4 H), 3.71 (br s, 6 H), 2.84 (m, 1 H), 2.68 (m, 1 H), 2.60 (br d, J = 6.85 Hz, 2 H), 2.33 (br s, 1 H), 2.16 (m, 1 H), 1.85 (br d, J = 12.23 Hz, 1 H), 1.69 (br d, J = 11.98 Hz, 3 H), 1.59 (br s, 1 H), 1.31 (m, 2 H), 1.05 (m, 2 H). LC/MS: m/z [M + H]⁺ 466.2, Rt (I = 220 nm): 1.81 min (LC/MS method A), calc. m/z: 466.23. |
| 208 | 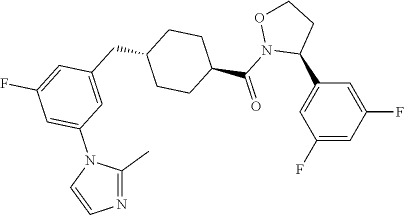 [SMILES: Cc1nccn1-c1cc(F)cc(C[C@H]2CC[C@H](C(=O)N3OCC[C@H]3c3cc(F)cc(F)c3)CC2)c1] | 1H NMR (400.23 MHz, DMSO-d6) d ppm 7.89 (d, J = 2.08 Hz, 1 H), 7.76 (d, J = 2.08 Hz, 1 H), 7.44 (br d, J = 9.17 Hz, 1 H), 7.35 (br d, J = 9.78 Hz, 1 H), 7.32 (s, 1 H), 7.13 (t, J = 8.24, 8.24 Hz, 1 H), 6.96 (br d, J = 6.48 Hz, 2 H), 5.32 (m, 1 H), 4.24 (td, J = 7.73, 7.73, 3.00 Hz, 1 H), 3.84 (m, 1 H), 2.86 (m, 1 H), 2.68 (m, 1 H), 2.60 (br d, J = 6.85 Hz, 2 H), 2.33 (s, 1 H), 2.18 (m, 1 H), 1.89 (br d, J = 12.47 Hz, 1 H), 1.70 (br d, J = 11.86 Hz, 3 H), 1.60 (br s, 1 H), 1.32 (m, 2 H), 1.06 (m, 2 H). LC/MS: m/z [M + H]⁺ 484.2, Rt (I = 220 nm): 1.87 min (LC/MS method A), calc. m/z: 484.22. |
| 209 | 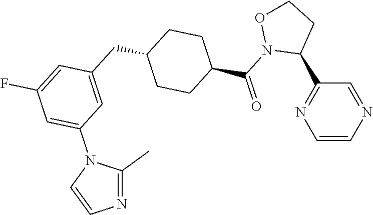 [SMILES: Cc1nccn1-c1cc(F)cc(C[C@H]2CC[C@H](C(=O)N3OCC[C@H]3c3cnccn3)CC2)c1] | 1H NMR (400.23 MHz, DMSO-d6) d ppm 8.58 (m, 3 H), 7.90 (d, J = 2.08 Hz, 1 H), 7.77 (d, J = 1.96 Hz, 1 H), 7.44 (br d, J = 9.17 Hz, 1 H), 7.35 (br d, J = 9.66 Hz, 1 H), 7.32 (s, 1 H), 5.40 (dd, J = 8.74, 6.17 Hz, 1 H), 4.30 (td, J = 7.76, 7.76, 3.67 Hz, 1 H), 3.93 (m, 1 H), 2.81 (m, 1 H), 2.61 (m, 3 H), 1.84 (br d, J = 11.86 Hz, 1 H), 1.71 (br s, 3 H), 1.59 (br s, 1 H), 1.30 (m, 2 H), 1.05 (m, 2 H). LC/MS: m/z [M + H]⁺ 450.2, Rt (I = 220 nm): 1.44 min (LC/MS method A), calc. m/z: 450.23. |
| 210 | 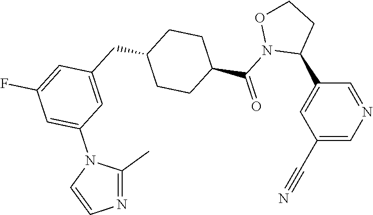 [SMILES: Cc1nccn1-c1cc(F)cc(C[C@H]2CC[C@H](C(=O)N3OCC[C@H]3c3cncc(C#N)c3)CC2)c1] | 1H NMR (400.23 MHz, DMSO-d6) d ppm 8.94 (d, J = 1.83 Hz, 1 H), 8.76 (d, J = 2.08 Hz, 1 H), 8.17 (t, J = 1.90, 1.90 Hz, 1 H), 7.90 (d, J = 2.08 Hz, 1 H), 7.77 (d, J = 2.08 Hz, 1 H), 7.44 (br d, J = 9.05 Hz, 1 H), 7.36 (br d, J = 9.54 Hz, 1 H), 7.33 (s, 1 H), 5.39 (m, 1 H), 4.29 (td, J = 7.67, 7.67, 2.87 Hz, 1 H), 3.89 (m, 4H), 3.57 (s, 3H), 2.89 (dddd, J = 12.10, 9.14, 6.45, 3.00 Hz, 1 H), 2.69 (m, 1 H), 2.60 (br d, J = 6.97 Hz, 2 H), 2.29 (m, 1 H), 1.88 (br d, J = 11.86 Hz, 1 H), 1.71 (br d, J = 11 Hzm 2H), 1.60 (m, 1H), 1.30 (m, 2H), 1.05 (m, 2H). LC/MS: m/z [M + H]⁺ 474.2, Rt (I = 220 nm): 1.55 min (LC/MS method A), calc, m/z: 474.23. |
| 211 | 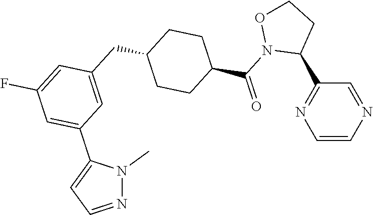 [SMILES: Cn1nccc1-c1cc(F)cc(C[C@H]2CC[C@H](C(=O)N3OCC[C@H]3c3cnccn3)CC2)c1] | 1H NMR (400.23 MHz, DMSO-d6) d ppm 8.58 (m, 3 H), 7.47 (d, J = 1.83 Hz, 1 H), 7.21 (br d, J = 9.54 Hz, 1 H), 7.17 (s, 1 H), 7.10 (br d, J = 9.78 Hz, 1 H), 6.45 (d, J = 1.83 Hz, 1 H), 5.40 (dd, J = 8.62, 6.17 Hz, 1 H), 4.30 (td, J = 7.61, 7.61, 3.61 Hz, 1 H), 3.93 (m, 2 H), 3.87 (s, 3 H), 2.81 (m, 1 H), 2.66 (m, 1 H), 2.56 (d, J = 7.09 Hz, 1 H), 1.83 (br d, J = 11.98 Hz, 1 H), 1.70 (br d, J = 10.88 Hz, 3 H), 1.57 (br s, 1 H), 1.29 (m, 2 H), 1.05 (m, 2 H). LC/MS: m/z [M + H]⁺ 450.2, Rt (I = 220 nm): 2.24 min (LC/MS method A), calc. m/z: 450.23. |
| 212 | 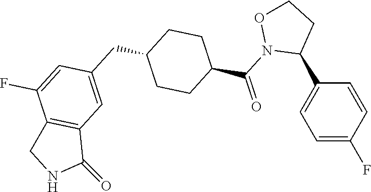 [SMILES: O=C1NCc2c(F)cc(C[C@H]3CC[C@H](C(=O)N4OCC[C@H]4c4ccc(F)cc4)CC3)cc21] | 1H NMR (400.23 MHz, DMSO-d6) d ppm 8.69 (s, 1 H), 7.29 (m, 4 H), 7.15 (t, J = 8.26, 8.26 Hz, 2 H), 5.29 (br t, J = 7.34, 7.34 Hz, 1 H), 4.40 (s, 2 H), 4.23 (td, J = 7.64, 7.64, 2.81 Hz, 1 H), 3.82 (m, under water peak), 2.84 (m, 1 H), 2.66 (m, 1 H), 2.60 (br d, J = 6.97 Hz, 2 H), 2.15 (m, 1 H), 1.84 (br d, J = 11.98 Hz, 1 H), 1.66 (br d, J = 11.98 Hz, 3 H), 1.54 (br s, 1 H), 1.29 (m, 2 H), 1.03 (m, 2 H). LC/MS: m/z [M + H]⁺ 441.2, Rt (I = 220 nm): 2.25 min (LC/MS method A), calc. m/z: 441.20. |
| 213 | 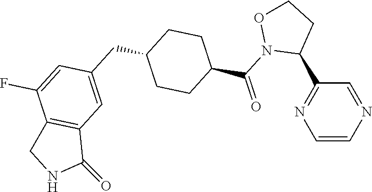 [SMILES: O=C1NCc2c(F)cc(C[C@H]3CC[C@H](C(=O)N4OCC[C@H]4c4cnccn4)CC3)cc21] | 1H NMR (400.23 MHz, DMSO-d6) d ppm 8.69 (s, 1 H), 8.58 (m, 3 H), 7.31 (s, 1 H), 7.26 (d, J = 10.39 Hz, 1 H), 5.39 (dd, J = 8.80, 6.24 Hz, 1 H), 4.40 (s, 2 H), 4.30 (td, J = 7.64, 7.64, 3.42 Hz, 1 H), 3.93 (m, 1 H), 2.81 (m, 1 H), 2.66 (br d, J = 8.07 Hz, 1 H), 2.60 (br d, J = 6.97 Hz, 2 H), 1.82 (br d, J = 13.20 Hz, 1 H), 1.66 (br d, J = 14.55 Hz, 3 H), 1.53 (br s, 1 H), 1.27 (m, 2 H), 1.03 (m, 2 H). LC/MS: m/z [M + H]⁺ 425.2, Rt (I = 220 nm): 1.78 min (LC/MS method A), calc. m/z: 425.20. |
| 214 | 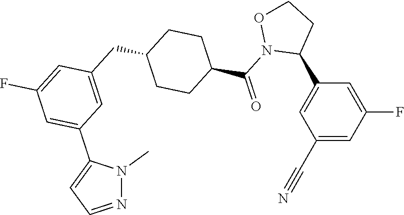 [SMILES: Cn1nccc1-c1cc(F)cc(C[C@H]2CC[C@H](C(=O)N3OCC[C@H]3c3cc(F)cc(C#N)c3)CC2)c1] | ¹H NMR (400 MHz, DMSO-d₆): δ ppm 7.76 (br d, J = 8.56 Hz, 1 H), 7.59 (s, 1 H), 7.47 (d, J = 1.83 Hz, 1 H), 7.45 (s, 1 H), 7.21 (br d, J = 9.29 Hz, 1 H), 7.18 (s, 1 H), 7.10 (br d, J = 9.78 Hz, 1 H), 6.45 (d, J = 1.83 Hz, 1 H), 5.36 (m, 1 H), 4.25 (m, 1 H), 3.87 (m, 4 H), 2.87 (m, 1 H), 2.67 (m, 1 H), 2.57 (d, J = 7.09 Hz, 2 H), 2.21 (m, 1 H), 1.89 (br d, J = 13.20 Hz, 1 H), 1.70 (br d, J = 10.03 Hz, 3 H), 1.58 (br s, 1 H), 1.31 (m, 2 H), 0.97 (m, 2 H). LC/MS: m/z [M + H]⁺ 491.2, Rt (I = 220 nm): 2.65 min (LC/MS method A), calc. m/z: 491.23. |
| 215 | 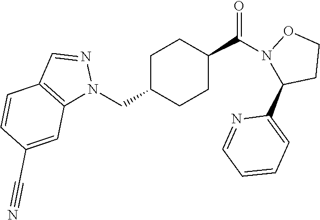 [SMILES: N#Cc1ccc2cnn(C[C@H]3CC[C@H](C(=O)N4OCC[C@H]4c4ccccn4)CC3)c2c1] | 1H NMR (400.23 MHz, DMSO-d6) d ppm 8.52 (d, J = 4.40 Hz, 1 H), 8.46 (s, 1 H), 8.25 (s, 1 H), 7.97 (m, 1 H), 7.79 (td, J = 7.64, 7.64, 1.71 Hz, 1 H), 7.45 (m, 1 H), 7.30 (m, 2 H), 5.32 (dd, J = 8.74, 5.93 Hz, 1 H), 4.35 (d, J = 6.97 Hz, 2 H), 4.23 (td, J = 7.55, 7.55, 3.36 Hz, 1 H), 3.87 (m, 1 H), 2.79 (tdd, J = 12.18, 12.18, 3.67, 3.39 Hz, 1 H), 2.67 (br s, 1 H), 2.42 (m, 1 H), 1.91 (br s, 1 H), 1.84 (br d, J = 11.98 Hz, 1 H), 1.68 (br s, 1 H), 1.54 (br t, J = 10.03, 10.03 Hz, 2 H), 1.27 (m, 2H, 1.14 (m, 2H). LC/MS: m/z [M + H]⁺ 416.2, Rt (I = 220 nm): 1.95 min (LC/MS method A), calc. m/z: 416.21. |
| 216 | 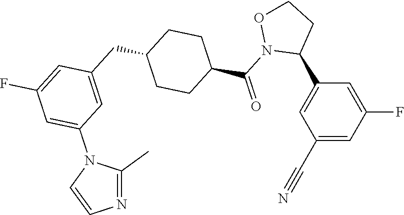 [SMILES: Cc1nccn1-c1cc(F)cc(C[C@H]2CC[C@H](C(=O)N3OCC[C@H]3c3cc(F)cc(C#N)c3)CC2)c1] | 1H NMR (400.23 MHz, DMSO-d6) d ppm 7.89 (d, J = 1.96 Hz, 1 H), 7.77 (d, J = 1.47 Hz, 1 H), 7.76 (m, 1 H), 7.58 (s, 1 H), 7.45 (br t, J = 8.56, 8.56 Hz, 2 H), 7.35 (br d, J = 9.54 Hz, 1 H), 7.33 (s, 1 H), 5.35 (m, 1 H), 4.25 (td, J = 7.73, 7.73, 2.75 Hz, 1 H), 3.86 (m, 1 H), 2.88 (m, 1 H), 2.69 (m, 1 H), 2.60 (br d, J = 6.85 Hz, 2 H), 2.33 (br s, 1 H), 2.22 (m, 1 H), 1.90 (br d, J = 11.13 Hz, 1 H), 1.71 (br d, J = 10.39 Hz, 3 H), 1.60 (br s, 1 H), 1.32 (m, 2 H), 1.05 (m, 2 H). LC/MS: m/z [M + H]⁺ 491.3, Rt (I = 220 nm): 1.78 min (LC/MS method A), calc. m/z: 491.23. |
| 217 | 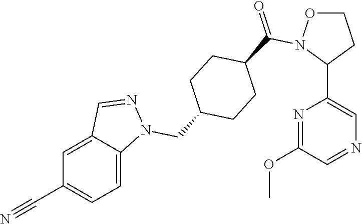 [SMILES: COc1cncc(C2CCON2C(=O)[C@H]2CC[C@H](Cn3ncc4cc(C#N)ccc43)CC2)n1] | 1H NMR (400.23 MHz, DMSO-d6) d ppm 8.40 (s, 1 H), 8.27 (s, 1 H), 8.20 (s, 1 H), 8.10 (s, 1 H), 7.91 (d, J = 8.80 Hz, 1 H), 7.71 (dd, J = 8.80, 1.34 Hz, 1 H), 5.30 (br dd, J = 8.74, 5.56 Hz, 2 H), 5.19 (br s, 1 H), 4.97 (br s, 2 H), 4.34 (d, J = 6.97 Hz, 2 H), 4.28 (m, 1 H), 3.93 (m, 1 H), 3.88 (s, 3 H), 2.77 (m, 1 H), 2.66 (br d, J = 11.74 Hz, 1 H), 2.42 (m, 1 H), 1.86 (m, 2 H), 1.69 (br d, J = 11.86 Hz, 1 H), 1.54 (br s, 2 H), 1.27 (m, 2 H), 1.13 (m, 2 H). LC/MS: m/z [M + H]⁺ 447.2, Rt (I = 220 nm): 2.1 min (LC/MS method A), calc. m/z: 447.22. |
| 218 | 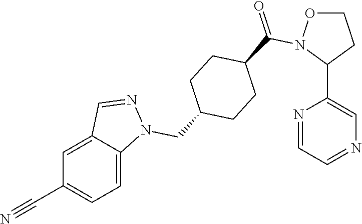 [SMILES: N#Cc1ccc2c(cnn2C[C@H]2CC[C@H](C(=O)N3OCCC3c3cnccn3)CC2)c1] | 1H NMR (400.23 MHz, DMSO-d6) d ppm 8.57 (m, 3 H), 8.40 (s, 1 H), 8.27 (s, 1 H), 7.91 (d, J = 8.68 Hz, 1 H), 7.71 (dd, J = 8.80, 1.34 Hz, 1 H), 5.38 (dd, J = 8.74, 6.17 Hz, 1 H), 4.79 (br s, 2 H), 4.34 (d, J = 6.97 Hz, 2 H), 4.29 (td, J = 7.76, 7.76, 3.30 Hz, 1 H), 3.92 (m, 1 H), 2.80 (m, 1 H), 2.64 (m, 1 H), 1.88 (m, 1 H), 1.82 (br d, J = 13.33 Hz, 1 H), 1.70 (br d, J = 11.37 Hz, 1 H), 1.54 (br d, J = 11.98 Hz, 2 H), 1.26 (m, 2 H), 1.12 (m, 2 H). LC/MS: m/z [M + H]⁺ 417.2, Rt [I = 220 nm): 1.85 min (LC/MS method A), calc. m/z: 417.20. |
| 219 | 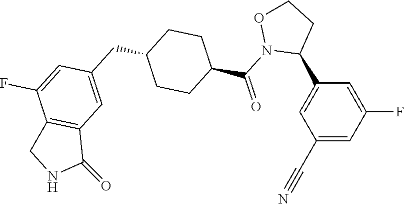 [SMILES: N#Cc1cc(F)cc([C@@H]2CCON2C(=O)[C@H]2CC[C@H](Cc3cc(F)c4c(c3)C(=O)NC4)CC2)c1] | ¹H NMR (400 MHz, DMSO-d₆): δ ppm 8.69 (s, 1 H), 7.75 (br d, J = 7.46 Hz, 1 H), 7.59 (s, 1 H), 7.46 (br d, J = 9.66 Hz, 1 H), 7.32 (s, 1 H), 7.26 (d, J = 10.15 Hz, 1 H), 5.35 (m, 1 H), 4.40 (s, 2 H), 4.25 (td, J = 7.64, 7.64, 2.81 Hz, 1 H), 3.81 (m, 1 H), 2.87 (m, 1 H), 2.66 (m, 1 H), 2.60 (br d, J = 6.85 Hz, 2 H), 2.21 (m, 1 H), 1.88 (br d, J = 12.47 Hz, 1 H), 1.68 (m, 3 H), 1.55 (br s, 1 H), 1.30 (m, 2 H), 1.03 (m, 2 H). LC/MS: m/z [M + H]⁺ 466.2, Rt (I = 220 nm): 2.22 min (LC/MS method A), calc. m/z: 466.19. |
| 220 | 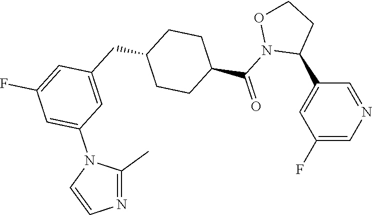 [SMILES: Cc1nccn1-c1cc(F)cc(C[C@H]2CC[C@H](C(=O)N3OCC[C@H]3c3cncc(F)c3)CC2)c1] | 1H NMR (400.23 MHz, DMSO-d6) d ppm 8.49 (d, J = 2.69 Hz, 1 H), 8.38 (s, 1 H), 7.90 (d, J = 2.08 Hz, 1 H), 7.77 (d, J = 2.08 Hz, 1 H), 7.57 (br d, J = 9.78 Hz, 1 H), 7.44 (br d, J = 9.05 Hz, 1 H), 7.34 (m, 2 H), 5.38 (m, 1 H), 4.28 (td, J = 7.70, 7.70, 2.93 Hz, 1 H), 3.88 (m, 4 H), 2.89 (m, 1 H), 2.68 (m, 1 H), 2.60 (br d, J = 6.97 Hz, 3 H), 2.28 (m, 1 H), 1.88 (br d, J = 12.84 Hz, 1 H), 1.70 (br d, J = 11.74 Hz, 3 H), 1.60 (br s, 1 H), 1.31 (br d, J = 13.94 Hz, 2 H), 1.06 (m, 2 H). LC/MS: m/z [M + H]⁺ 467.2, Rt (I = 220 nm): 1.57 min (LC/MS method A), calc. m/z: 467.23. |
| 221 | 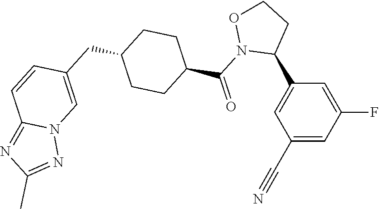 [SMILES: Cc1nc2ccc(C[C@H]3CC[C@H](C(=O)N4OCC[C@H]4c4cc(F)cc(C#N)c4)CC3)cn2n1] | 1H NMR (400.23 MHz, DMSO-d6) d ppm 8.68 (s, 1 H), 7.76 (d, J = 8.64 Hz, 1 H), 7.66 (d, J = 9.05 Hz, 1 H), 7.59 (s, 1 H), 7.54 (d, J = 9.08 Hz, 1 H), 7.46 (br d, J = 9.66 Hz, 1 H), 5.36 (dd, J = 8.44, 6.60 Hz, 1 H), 4.40 (br s, 8 H), 4.25 (td, J = 7.67, 7.67, 2.87 Hz, 5 H), 3.87 (m, 1 H), 2.87 (dddd, J = 12.15, 9.15, 6.51, 3.12 Hz, 1 H), 2.67 (m, 1 H), 2.57 (d, J = 7.09 Hz, 2 H), 2.21 (m, 1 H), 1.89 (br d, J = 12.10 Hz, 1 H), 1.70 (br d, J = 11.74 Hz, 3 H), 1.57 (m, 1 H), 1.30 (m, 2 H), 1.06 (m, 2 H). LC/MS: m/z [M + H]⁺ 448.2, Rt (I = 220 nm): 2.04 min (LC/MS method A), calc. m/z: 448.22. |
| 222 | 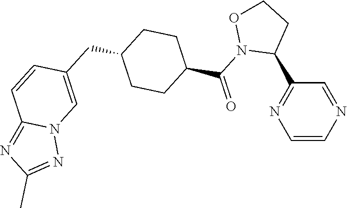 [SMILES: Cc1nc2ccc(C[C@H]3CC[C@H](C(=O)N4OCC[C@H]4c4cnccn4)CC3)cn2n1] | 1H NMR (400.23 MHz, DMSO-d6) d ppm 8.67 (s, 1 H), 8.58 (m, 3 H), 7.65 (d, J = 9.05 Hz, 1 H), 7.52 (d, J = 9.29 Hz, 1 H), 5.39 (dd, J = 8.68, 6.24 Hz, 1 H), 4.30 (td, J = 7.55, 7.55, 3.61 Hz, 4 H), 3.93 (m, 1 H), 2.81 (m, 1 H), 2.65 (m, 1 H), 2.56 (d, J = 6.85 Hz, 2 H), 1.83 (br d, J = 12.47 Hz, 1 H), 1.69 (br d, J = 12.0 Hz, 3 H), 1.57 (br s, 1 H), 1.29 (m, 2 H), 1.04 (m, 2 H). LC/MS: m/z [M + H]⁺ 407.2, Rt (I = 220 nm): 1.57 min (LC/MS method A), calc. m/z: 407.22. |
| 223 | 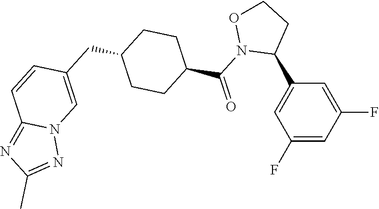 [SMILES: Cc1nc2ccc(C[C@H]3CC[C@H](C(=O)N4OCC[C@H]4c4cc(F)cc(F)c4)CC3)cn2n1] | ¹H NMR (400 MHz, DMSO-d₆): δ ppm 8.66 (s, 1 H), 7.64 (d, J = 9.05 Hz, 1 H), 7.51 (d, J = 9.05 Hz, 1 H), 7.12 (t, J = 9.22, 9.22 Hz, 1 H), 6.96 (br d, J = 6.72 Hz, 2 H), 5.31 (m, 1 H), 4.23 (m, 1 H), 3.84 (m, 1 H), 2.85 (m, 1 H), 2.67 (br s, 1 H), 2.56 (s, 3 H), 2.33 (br s, 1 H), 2.17 (m, 1 H), 1.88 (br d, J = 11.49 Hz, 1 H), 1.69 (m, 3 H), 1.58 (br s, 1 H), 1.31 (m, 2 H), 1.05 (m, 2 H). LC/MS: m/z [M + H]⁺ 441.2, Rt (I = 220 nm): 2.18 min (LC/MS method A), calc. m/z: 441.21. |
| 224 | 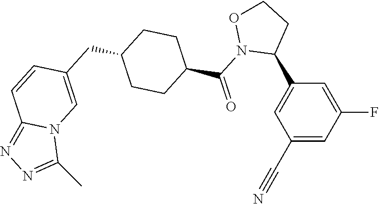 [SMILES: Cc1nnc2ccc(C[C@H]3CC[C@H](C(=O)N4OCC[C@H]4c4cc(F)cc(C#N)c4)CC3)cn12] | 1H NMR (400.23 MHz, DMSO-d6) d ppm 7.79 (br dd, J = 19.50, 9.11 Hz, 1 H), 7.58 (s, 1 H), 7.46 (br d, J = 8.68 Hz, 1 H), 5.35 (m, 1 H), 4.25 (m, 1 H), 3.86 (m, 3 H), 2.88 (br s, 1 H), 2.72 (s, 1 H), 2.67 (br s, 1 H), 2.57 (br d, J = 6.97 Hz, 1 H), 2.33 (br s, 1 H), 2.23 (br s, 1 H), 1.90 (br d, J = 8.93 Hz, 1 H), 1.71 (br s, 2 H), 1.31 (br d, J = 12.10 Hz, 1 H), 1.08 (br d, J = 13.57 Hz, 1 H). LC/MS: m/z [M + H]⁺ 448.2, Rt (I = 220 nm): 1.76 min (LC/MS method A), calc. m/z: 448.22. |
| 225 | 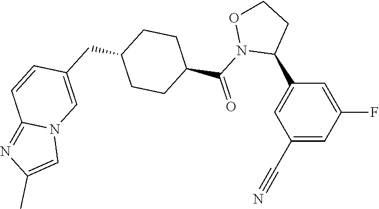 [SMILES: Cc1cn2cc(C[C@H]3CC[C@H](C(=O)N4OCC[C@H]4c4cc(F)cc(C#N)c4)CC3)ccc2n1] | ¹H NMR (400 MHz, DMSO-d₆): δ 8.67 (s, 1 H), 8.00 (s, 1 H), 7.80 (m, 3 H), 7.58 (s, 1 H), 7.46 (br d, J = 9.41 Hz, 1 H), 5.35 (m, 1 H), 4.25 (m, 1 H), 3.86 (m, 1 H), 2.88 (m, 1 H), 2.68 (br d, J = 7.70 Hz, 1 H), 2.61 (br d, J = 6.97 Hz, 2 H), 2.33 (br s, 1 H), 2.22 (m, 1 H), 1.90 (br d, J = 12.10 Hz, 1 H), 1.71 (br d, J = 11.62 Hz, 3 H), 1.59 (br, s, 1 H), 1.31 (m, 2 H), 1.07 (m, 2 H). LC/MS: m/z [M + H]⁺ 447.2, Rt (I = 220 nm): 1.56 min (LC/MS method A), calc. m/z: 447.22. |
| 226 | 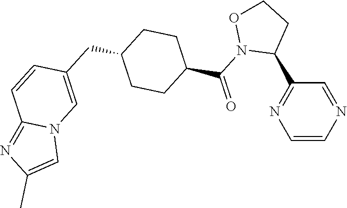 [SMILES: Cc1cn2cc(C[C@H]3CC[C@H](C(=O)N4OCC[C@H]4c4cnccn4)CC3)ccc2n1] | 1H NMR (400.23 MHz, DMSO-d6) d ppm 14.21 (br s, 1 H), 8.67 (s, 1 H), 8.58 (m, 3 H), 8.00 (s, 1 H), 7.81 (q, J = 9.17, 9.17 Hz, 2 H), 5.39 (dd, J = 8.62, 6.30 Hz, 1 H), 4.30 (td, J = 7.70, 7.70, 3.67 Hz, 2 H), 3.93 (m, 1 H), 2.81 (m, 1 H), 2.62 (m, 3 H), 2.35 (m, 1 H), 1.84 (br d, J = 13.20 Hz, 1 H), 1.71 (br s, 3 H), 1.58 (br s, 1 H), 1.29 (m, 2 H), 1.06 (m, 2 H). LC/MS: m/z [M + H]⁺ 406.2, Rt (I = 220 nm): 1.16 min (LC/MS method A), calc. m/z: 406.22. |
| 227 | 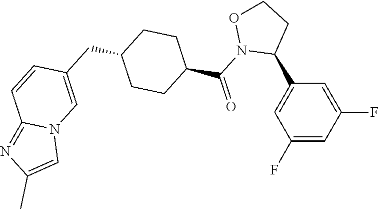 [SMILES: Cc1cn2cc(C[C@H]3CC[C@H](C(=O)N4OCC[C@H]4c4cc(F)cc(F)c4)CC3)ccc2n1] | ¹H NMR (400 MHz, DMSO-d₆): δ ppm 14.21 (br s, 1 H), 8.67 (s, 1 H), 8.00 (s, 1 H), 7.81 (m, 2 H), 7.12 (br t, J = 9.47, 9.47 Hz, 1 H), 6.96 (br d, J = 6.60 Hz, 2 H), 5.31 (m, 1 H), 4.23 (m, 1 H), 4.21 (br s, 1 H), 3.84 (m, 1 H), 2.85 (m, 1 H), 2.67 (br s, 1 H), 2.61 (br d, J = 6.97 Hz, 3 H), 2.18 (m, 1 H), 1.89 (br d, J = 12.35 Hz, 1 H), 1.70 (br d, J = 10.27 Hz, 3 H), 1.59 (br s, 1 H), 1.31 (m, 2 H), 1.07 (m, 2 H). LC/MS: m/z [M + H]⁺ 440.2, Rt (I = 220 nm): 1.64 min (LC/MS method A), calc. m/z: 440.22. |
| 228 | 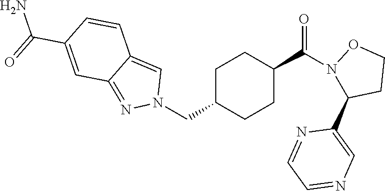 [SMILES: NC(=O)c1ccc2cn(C[C@H]3CC[C@H](C(=O)N4OCC[C@H]4c4cnccn4)CC3)nc2c1] | 1H NMR (400.23 MHz, DMSO-d6) d ppm 8.58 (m, 3 H), 8.39 (s, 1 H), 8.19 (s, 1 H), 7.98 (br s, 1 H), 7.72 (d, J = 8.68 Hz, 1 H), 7.51 (dd, J = 8.74, 1.16 Hz, 1 H), 7.29 (br s, 1 H), 5.39 (dd, J = 8.68, 6.36 Hz, 1 H), 4.30 (m, 4 H), 3.93 (m, 4 H), 2.81 (m, 1 H), 2.66 (m, 1 H), 1.97 (br s, 1 H), 1.84 (br d, J = 11.62 Hz, 1 H), 1.72 (br d, J = 11.37 Hz, 1 H), 1.57 (br d, J = 11.49 Hz, 2 H), 1.29 (m, 2 H), 1.12 (m, 2 H). LC/MS: m/z [M + H]⁺ 435.2, Rt (l = 220 nm): 1.38 min (LC/MS method A), calc. m/z: 435.22. |
| 229 | 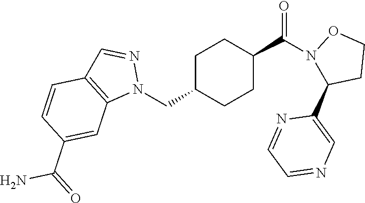 [SMILES: NC(=O)c1ccc2cnn(C[C@H]3CC[C@H](C(=O)N4OCC[C@H]4c4cnccn4)CC3)c2c1] | 1H NMR (400.23 MHz, DMSO-d6) d ppm 8.58 (m, 3 H), 8.21 (s, 1 H), 8.12 (s, 1 H), 8.04 (br s, 1 H), 7.79 (d, J = 8.44 Hz, 1 H), 7.63 (dd, J = 8.44, 1.10 Hz, 1 H), 7.45 (br s, 1 H), 5.39 (dd, J = 8.74, 6.17 Hz, 1 H), 4.92 (br s, 2 H), 4.30 (br d, J = 7.09 Hz, 3 H), 4.27 (br d, J = 3.55 Hz, 1 H), 3.92 (m, 1 H), 2.80 (m, 1 H), 2.66 (m, 1 H), 1.93 (br s, 1 H), 1.83 (br d, J = 13.33 Hz, 1 H), 1.71 (br d, J = 9.29 Hz, 1 H), 1.56 (br s, 2 H), 1.25 (m, 2 H), 1.15 (br d, J = 12.23 Hz, 1 H). LC/MS: m/z [M + H]⁺ 435.2, Rt (I = 220 nm): 1.46 min (LC/MS method A), calc. m/z: 435.22. |
| 230 | 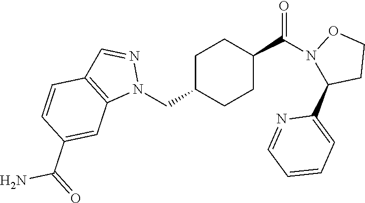 [SMILES: NC(=O)c1ccc2cnn(C[C@H]3CC[C@H](C(=O)N4OCC[C@H]4c4ccccn4)CC3)c2c1] | 1H NMR (400.23 MHz, DMSO-d6) d ppm 8.52 (d, J = 4.28 Hz, 1 H), 8.21 (s, 1 H), 8.12 (s, 1 H), 8.04 (br s, 1 H), 7.78 (m, 2 H), 7.63 (d, J = 8.56 Hz, 1 H), 7.45 (br s, 1 H), 7.29 (m, 2 H), 5.33 (br d, J = 6.24 Hz, 1 H), 4.31 (br d, J = 7.09 Hz, 2 H), 4.23 (br d, J = 3.67 Hz, 1 H), 2.67 (s, 2 H), 2.42 (br d, J = 13.57 Hz, 1 H), 2.33 (s, 1 H), 1.84 (br s, 2 H), 1.56 (br s, 3 H), 1.29 (br d, J = 12.10 Hz, 2 H). LC/MS: m/z [M + H]⁺ 434.2, Rt (I = 220 nm): 1.48 min (LC/MS method A), calc. m/z: 434.22. |
| 231 | 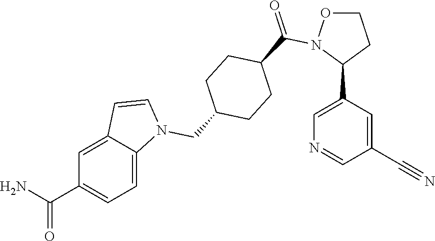 [SMILES: N#Cc1cncc([C@@H]2CCON2C(=O)[C@H]2CC[C@H](Cn3ccc4cc(C(N)=O)ccc43)CC2)c1] | 1H NMR (400.23 MHz, DMSO-d6) d ppm 8.92 (d, J = 1.83 Hz, 1 H), 8.75 (d, J = 2.08 Hz, 1 H), 8.16 (m, 3 H), 8.04 (br s, 1 H), 7.79 (d, J = 8.44 Hz, 1 H), 7.63 (d, J = 7.92 Hz, 1 H), 7.45 (br s, 1 H), 5.39 (m, 1 H), 4.29 (m, 4 H, under water peak), 3.89 (m, 1 H), 2.88 (m, 1 H), 2.67 (br d, J = 1.96 Hz, 1 H), 2.29 (m, 1 H), 1.93 (br s, 1 H), 1.88 (br d, J = 12.35 Hz, 1 H), 1.71 (br d, J = 12.72 Hz, 1 H), 1.56 (br t, J = 13.33, 13.33 Hz, 2 H), 1.27 (m, 2 H), 1.16 (br d, J = 12.96 Hz, 1 H), 1.11 (br s, 1 H). LC/MS: m/z [M + H]⁺ 459.2, Rt (I = 220 nm): 1.59 min (LC/MS method A), calc. m/z: 459.22. |
| 232 | 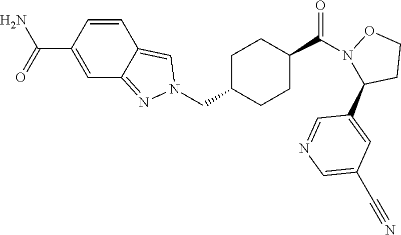 [SMILES: N#Cc1cncc([C@@H]2CCON2C(=O)[C@H]2CC[C@H](Cn3cc4ccc(C(N)=O)cc4n3)CC2)c1] | 1H NMR (400.23 MHz, DMSO-d6) d ppm 8.93 (d, J = 1.96 Hz, 1 H), 8.76 (d, J = 2.08 Hz, 1 H), 8.39 (s, 1 H), 8.19 (s, 1 H), 8.18 (s, 1 H), 7.98 (br s, 1 H), 7.72 (d, J = 8.68 Hz, 1 H), 7.51 (dd, J = 8.74, 1.28 Hz, 1 H), 7.29 (br s, 1 H), 5.39 (m, 1 H), 4.32 (br d, J = 6.85 Hz, 4 H), 3.89 (m, 4 H), 2.88 (m, 1 H), 2.67 (br s, 1 H), 2.30 (m, 1 H), 1.99 (br d, J = 11.86 Hz, 1 H), 1.89 (br d, J = 13.08 Hz, 2 H), 1.72 (br d, J = 11.62 Hz, 1 H), 1.57 (bs, xH), 1.37 (s, 2H), 1.30 (m, 2H), 1.12 (m, 2H). LC/MS: m/z [M + H]⁺ 459.2, Rt (I = 220 nm): 1.52 min (LC/MS method A), calc. m/z: 459.22. |
| 233 | 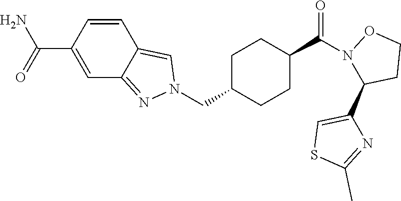 [SMILES: Cc1nc([C@@H]2CCON2C(=O)[C@H]2CC[C@H](Cn3cc4ccc(C(N)=O)cc4n3)CC2)cs1] | 1H NMR (400.23 MHz, DMSO-d6) d ppm 8.39 (s, 1 H), 8.19 (s, 1 H), 7.98 (br s, 1 H), 7.72 (d, J = 8.80 Hz, 1 H), 7.51 (d, J = 8.68 Hz, 1 H), 7.29 (br s, 1 H), 7.18 (s, 1 H), 5.34 (m, 1 H), 4.31 (br d, J = 6.97 Hz, 3 H), 4.25 (br s, 1 H), 4.21 (br dd, J = 7.58, 3.55 Hz, 4 H), 3.85 (m, 7 H), 2.70 (br d, J = 3.55 Hz, 1 H), 2.67 (br s, 1 H), 2.61 (s, 4 H), 2.38 (m, 2 H), 1.96 (br s, 1 H), 1.83 (br d, J = 11.86 Hz, 1 H), 1.68 (br s, 1 H), 1.57 (br d, J = 12.59 Hz, 2 H), 1.30 (br d, J = 12.23 Hz, 2 H), 1.10 (m, 2 H). LC/MS: m/z [M + H]⁺ 454.2, Rt (I = 220 nm): 1.56 min (LC/MS method A), calc. m/z 454.19. |
| 234 | 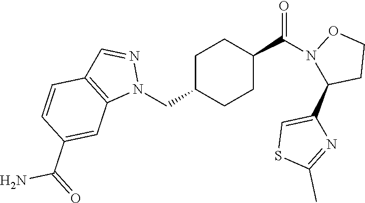 [SMILES: Cc1nc([C@@H]2CCON2C(=O)[C@H]2CC[C@H](Cn3ncc4ccc(C(N)=O)cc43)CC2)cs1] | 1H NMR (400.23 MHz, DMSO-d6) d ppm 8.21 (s, 1 H), 8.12 (s, 1 H), 8.04 (br s, 1 H), 7.79 (d, J = 8.56 Hz, 1 H), 7.63 (d, J = 8.44 Hz, 1 H), 7.45 (br s, 1 H), 7.17 (s, 1 H), 5.34 (m, 1 H), 4.30 (br d, J = 7.09 Hz, 5 H), 4.24 (br s, 1 H), 4.21 (br dd, J = 7.40, 3.73 Hz, 4 H), 3.84 (q, J = 7.87, 7.87, 7.87 Hz, 1 H), 2.69 (m, 1 H), 2.61 (s, 4 H), 2.38 (m, 3 H), 1.93 (br s, 1 H), 1.82 (br d, J = 12.47 Hz, 1 H), 1.66 (br s, 1 H), 1.55 (br s, 2 H), 1.27 (m, 2 H), 1.14 (m, 2 H). LC/MS: m/z [M + H]⁺ 454.2, Rt (I = 220 nm): 1.63 min (LC/MS method A), calc. m/z: 454.19. |
| 235 | 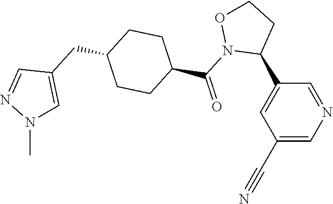 [SMILES: Cn1cc(C[C@H]2CC[C@H](C(=O)N3OCC[C@H]3c3cncc(C#N)c3)CC2)cn1] | 1H NMR (400.23 MHz, DMSO-d6) d ppm 8.93 (d, J = 1.83 Hz, 1 H), 8.76 (d, J = 2.20 Hz, 1 H), 8.18 (t, J = 1.90, 1.90 Hz, 1 H), 7.42 (s, 1 H), 7.18 (s, 1 H), 5.40 (dd, J = 8.62, 6.54 Hz, 1 H), 4.28 (td, J = 7.73, 7.73, 3.12 Hz, 5 H), 3.93 (br s, 1 H), 3.89 (m, 3 H), 3.76 (s, 4 H), 3.58 (s, 1 H), 2.89 (dddd, J = 12.15, 9.15, 6.45, 3.18 Hz, 1 H), 2.66 (m, 1 H), 2.30 (m, 1 H), 2.27 (d, J = 6.72 Hz, 3 H), 1.87 (br d, J = 12.10 Hz, 1 H), 1.72 (m, 3 H), 1.29 (m, 4 H), 0.96 (m, 2 H). LC/MS: m/z [M + H]⁺ 380.2, Rt (I = 220 nm): 1.78 min (LC/MS method A), calc. m/z: 380.21. |
| 236 | 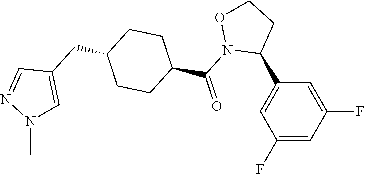 [SMILES: Cn1cc(C[C@H]2CC[C@H](C(=O)N3OCC[C@H]3c3cc(F)cc(F)c3)CC2)cn1] | ¹H NMR (400 MHz, DMSO-d₆): δ ppm 7.41 (s, 1 H), 7.18 (s, 1 H), 7.12 (tt, J = 9.29, 9.29, 2.32, 2.32 Hz, 1 H), 6.97 (m, 2 H), 5.32 (dd, J = 8.62, 6.42 Hz, 1 H), 4.23 (td, J = 7.79, 7.79, 3.00 Hz, 1 H), 3.83 (ddd, J = 9.29, 8.01, 6.66 Hz, 1 H), 3.76 (s, 3 H), 2.86 (m, 1 H), 2.67 (br d, J = 1.71 Hz, 1 H), 2.27 (d, J = 6.85 Hz, 2 H), 2.17 (m, 1 H), 1.88 (br d, J = 13.45 Hz, 1 H), 1.72 (m, 3 H), 1.31 (m, 3 H), 0.97 (m, 2 H). LC/MS: m/z [M + H]⁺ 390.2, Rt (I = 220 nm): 2.27 min (LC/MS method A), calc. m/z: 390.20. |
| 237 | 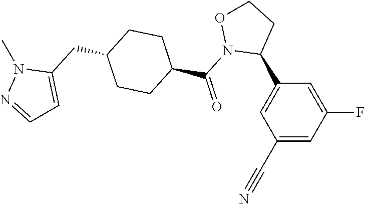 [SMILES: Cn1nccc1C[C@H]1CC[C@H](C(=O)N2OCC[C@H]2c2cc(F)cc(C#N)c2)CC1] | 1H NMR (400.23 MHz, DMSO-d6) δ ppm 7.76 (d, J = 8.67 Hz, 1 H), 7.59 (s, 1 H), 7.46 (br d, J = 9.54 Hz, 1 H), 7.28 (d, J = 1.71 Hz, 1 H), 6.01 (d, J = 1.59 Hz, 1 H), 5.75 (s, 1 H), 5.36 (dd, J = 8.38, 6.42 Hz, 1 H), 4.46 (br t, J = 10.33, 10.33 Hz, 1 H), 4.25 (td, J = 7.76, 7.76, 2.81 Hz, 5 H), 3.87 (br dd, J = 8.62, 7.40 Hz, 4 H), 3.84 (m, 4 H), 3.71 (s, 3 H), 2.88 (m, 1 H), 2.67 (br d, J = 1.71 Hz, 1 H), 2.21 (m, 1 H), 1.89 (br d, J = 12.35 Hz, 1 H), 1.74 (m, 3 H), 1.52 (br dd, J = 10.82, 3.97 Hz, 2H), 1.32 (m, 2H), 1.04 (m, 2H). LC/MS: m/z [M + H]⁺ 397.2, Rt (I = 220 nm): 2.08 min (LC/MS method A), calc. m/z: 397.20. |
| 238 | 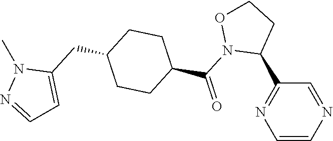 [SMILES: Cn1nccc1C[C@H]1CC[C@H](C(=O)N2OCC[C@H]2c2cnccn2)CC1] | 1H NMR (400.23 MHz, DMSO-d6) d ppm 8.61 (dd, J = 2.51, 1.53 Hz, 1 H), 8.57 (dd, J = 3.67, 1.96 Hz, 2 H), 7.28 (d, J = 1.71 Hz, 1 H), 6.01 (d, J = 1.71 Hz, 1 H), 5.40 (dd, J = 8.80, 6.11 Hz, 1 H), 4.30 (m, 4H), 3.93 (m, 4 H), 3.88 (br s, 1 H), 3.71 (s, 3 H), 2.82 (m, 1 H), 2.67 (m, 1 H), 1.84 (br d, J = 12.35 Hz, 1 H), 1.73 (br d, J = 10.88 Hz, 3 H), 1.52 (m, 1 H), 1.31 (m, 2 H), 1.05 (m, 2 H). LC/MS: m/z [M + H]⁺ 356.2, Rt (I = 220 nm): 1.56 min (LC/MS method A), calc. m/z: 356.21. |
| 239 | 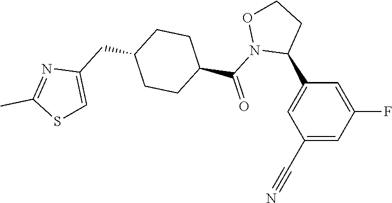 [SMILES: Cc1nc(C[C@H]2CC[C@H](C(=O)N3OCC[C@H]3c3cc(F)cc(C#N)c3)CC2)cs1] | ¹H NMR (400 MHz, DMSO-d₆): δ ppm 7.76 (d, J = 8.61 Hz, 1 H), 7.59 (s, 1 H), 7.46 (br d, J = 9.41 Hz, 1 H), 7.08 (s, 1 H), 5.36 (dd, J = 8.56, 6.48 Hz, 1 H), 4.25 (td, J = 7.76, 7.76, 2.93 Hz, 1 H), 3.88 (m, 1 H), 2.87 (m, 1 H), 2.62 (m, 1 H), 2.55 (s, 3 H), 2.21 (m, 1 H), 1.89 (br d, J = 12.84 Hz, 1 H), 1.67 (m, 4 H), 1.31 (m, 2 H), 1.03 (m, 2 H). LC/MS: m/z [M + H]⁺ 414.2, Rt (I = 220 nm): 2.24 min (LC/MS method A), calc. m/z: 414.17. |
| 240 | 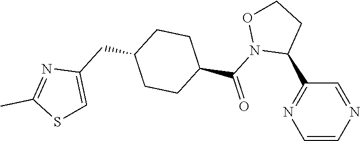 [SMILES: Cc1nc(C[C@H]2CC[C@H](C(=O)N3OCC[C@H]3c3cnccn3)CC2)cs1] | 1H NMR (400.23 MHz, DMSO-d6) d ppm 8.58 (m, 3 H), 7.07 (s, 1 H), 5.40 (dd, J = 8.80, 6.11 Hz, 1 H), 4.30 (td, J = 7.64, 7.64, 3.55 Hz, 1 H), 3.93 (m, 4H, under water peak), 2.81 (m, 1 H), 2.62 (s, 4 H), 1.83 (br d, J = 11.62 Hz, 1 H), 1.70 (br d, J = 12.35 Hz, 3 H), 1.62 (br d, J = 3.91 Hz, 1 H), 1.29 (m, 2 H), 1.02 (m, 2 H). LC/MS: m/z [M + H]⁺ 373.2, Rt (I = 220 nm): 1.67 min (LC/MS method A), calc. m/z: 373.17. |
| 241 | 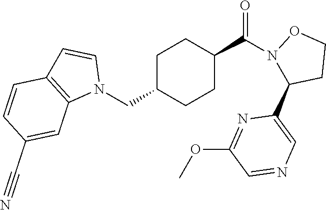 [SMILES: COc1cncc([C@@H]2CCON2C(=O)[C@H]2CC[C@H](Cn3ccc4ccc(C#N)cc43)CC2)n1] | LC/MS: m/z [M + H]⁺ 446.2, Rt (I = 220 nm): 2.38 min (LC/MS method A), calc. m/z: 446.22. |
| 242 | 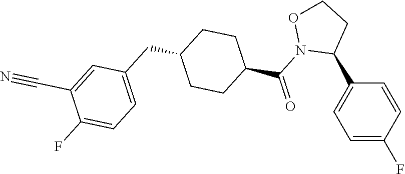 [SMILES: N#Cc1cc(C[C@H]2CC[C@H](C(=O)N3OCC[C@H]3c3ccc(F)cc3)CC2)ccc1F] | 1H NMR (400.23 MHz, DMSO-d6) d ppm 7.72 (dd, J = 6.36, 2.08 Hz, 1 H), 7.58 (m, 1 H), 7.43 (t, J = 8.83, 8.83 Hz, 1 H), 7.29 (dd, J = 8.62, 5.56 Hz, 2 H), 7.15 (t, J = 8.35, 8.35 Hz, 2 H), 5.29 (m, 1 H), 4.23 (td, J = 7.76, 7.76, 3.06 Hz, 1 H), 3.83 (m, 1 H), 3.80 (s, 1 H), 2.84 (m, 1 H), 2.66 (br d, J = 9.90 Hz, 1 H), 2.16 (m, 1 H), 1.84 (br d, J = 13.57 Hz, 1 H), 1.64 (br d, J = 12.35 Hz, 3 H), 1.51 (m, 1 H), 1.28 (m, 2 H), 1.00 (m, 2 H). LC/MS: m/z [M + H]⁺ 411.2, Rt (I = 220 nm): 2.65 min (LC/MS method A), calc. m/z: 411.19. |
| 243 | 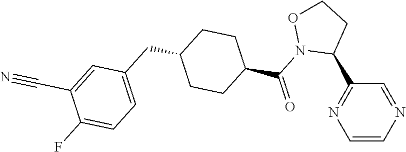 [SMILES: N#Cc1cc(C[C@H]2CC[C@H](C(=O)N3OCC[C@H]3c3cnccn3)CC2)ccc1F] | ¹H NMR (400 MHz, DMSO-d₆): δ ppm 8.58 (m, 3 H), 7.72 (dd, J = 6.24, 2.20 Hz, 1 H), 7.58 (m, 1 H), 7.43 (t, J = 9.05, 9.05 Hz, 1 H), 5.39 (dd, J = 8.80, 6.24 Hz, 1 H), 4.30 (td, J = 7.73, 7.73, 3.61 Hz, 1 H), 3.93 (m, 1 H), 2.81 (m, 1 H), 2.65 (m, 1 H), 1.82 (br d, J = 12.59 Hz, 1 H), 1.71 (br d, J = 12.10 Hz, 1 H), 1.64 (br d, J = 11.74 Hz, 2 H), 1.51 (br d, J = 3.55 Hz, 1 H), 1.28 (m, 2 H), 1.01 (m, 2 H). LC/MS: m/z [M + H]⁺ 395.2, Rt (I = 220 nm): 2.18 (LC/MS method A), calc. m/z: 395.19. |
| 244 | 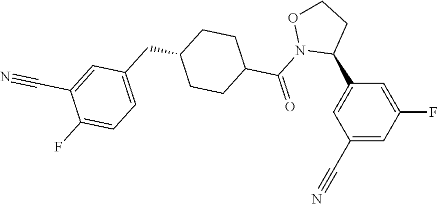 | ¹H NMR (400 MHz, DMSO-d₆): δ ppm 7.74 (m, 2 H), 7.59 (m, 2 H), 7.44 (m, 2 H), 5.35 (dd, J = 8.44, 6.48 Hz, 1 H), 4.25 (td, J = 7.67, 7.67, 2.87 Hz, 1 H), 3.87 (m, 1 H), 3.83 (s, 1 H), 2.87 (m, 1 H), 2.67 (m, 1 H), 2.21 (m, 1 H), 1.88 (br d, J = 12.72 Hz, 1 H), 1.66 (m, 3 H), 1.51 (br dd, J = 7.21, 3.67 Hz, 1 H), 1.30 (m, 2 H), 1.02 (m, 2 H). LC/MS: m/z [M + H]⁺ 436.1, Rt (I = 220 nm): 2.62 min (LC/MS method A), calc. m/z: 436.18. |
| 245 | 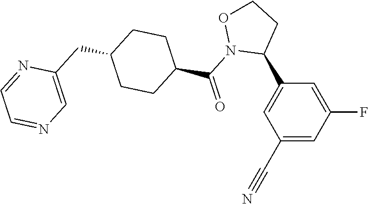 [SMILES: N#Cc1cc(F)cc([C@@H]2CCON2C(=O)[C@H]2CC[C@H](Cc3cnccn3)CC2)c1] | 1H NMR (400.23 MHz, DMSO-d6) d ppm 8.56 (m, 1 H), 8.52 (s, 1 H), 8.46 (d, J = 2.57 Hz, 1 H), 7.76 (br d, J = 8.56 Hz, 1 H), 7.59 (s, 1 H), 7.46 (br d, J = 9.41 Hz, 1 H), 5.36 (m, 1 H), 4.25 (td, J = 7.86, 7.86, 3.00 Hz, 1 H), 3.86 (m, 1 H), 2.87 (m, 1 H), 2.68 (d, J = 6.85 Hz, 4 H), 2.21 (m, 1 H), 1.89 (br d, J = 12.72 Hz, 1 H), 1.74 (br s, 1 H), 1.67 (br d, J = 12.72 Hz, 3 H), 1.30 (q, J = 12.39, 12.39, 12.39 Hz, 2 H), 1.09 (m, 2 H). LC/MS: m/z [M + H]⁺ 395.2, Rt (I = 220 nm): 1.99 min (LC/MS method A), calc. m/z: 395.19. |
| 246 | 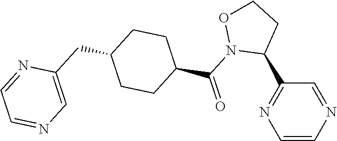 [SMILES: O=C([C@H]1CC[C@H](Cc2cnccn2)CC1)N1OCC[C@H]1c1cnccn1] | 1H NMR (400.23 MHz, DMSO-d6) d ppm 8.56 (m, 1 H), 8.52 (s, 1 H), 8.46 (d, J = 2.57 Hz, 1 H), 7.76 (br d, J = 8.56 Hz, 1 H), 7.59 (s, 1 H), 7.46 (br d, J = 9.41 Hz, 1 H), 5.36 (m, 1 H), 4.25 (td, J = 7.86, 7.86, 3.00 Hz, 1 H), 3.86 (m, 1 H), 2.87 (m, 1 H), 2.68 (d, J = 6.85 Hz, 4 H), 2.21 (m, 1 H), 1.89 (br d, J = 12.72 Hz, 1 H), 1.74 (br s, 1 H), 1.67 (br d, J = 12.72 Hz, 3 H), 1.30 (q, J = 12.39, 12.39, 12.39 Hz, 2 H), 1.09 (m, 2 H). LC/MS: m/z [M + H]⁺ 354.2, Rt (I = 220 nm): 1.46 min (LC/MS method A), calc. m/z: 354.19. |
| 247 | 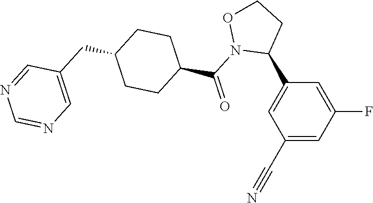 [SMILES: N#Cc1cc(F)cc([C@@H]2CCON2C(=O)[C@H]2CC[C@H](Cc3cncnc3)CC2)c1] | 1H NMR (400.23 MHz, DMSO-d6) d ppm 9.03 (s, 1 H), 8.65 (s, 2 H), 7.76 (d, J = 8.67 Hz, 1 H), 7.59 (s, 1 H), 7.46 (br d, J = 9.54 Hz, 1 H), 5.36 (dd, J = 8.50, 6.54 Hz, 1 H), 4.46 (m, 1 H), 4.25 (td, J = 7.73, 7.73, 3.00 Hz, 4 H), 3.87 (br dd, J = 8.68, 7.34 Hz, 2 H), 3.83 (s, 1 H), 2.87 (dddd, J = 12.13, 9.14, 6.48, 3.06 Hz, 1 H), 2.67 (br t, J = 11.80, 11.80 Hz, 1 H), 2.21 (m, 1 H), 1.89 (br d, J = 12.59 Hz, 1 H), 1.67 (br d, J = 12.59 Hz, 3 H), 1.56 (td, J = 7.27, 7.27, 4.16 Hz, 1 H), 1.30 (m, 2 H), 1.03 (m, 2H). LC/MS: m/z [M + H]⁺ 395.2, Rt (I = 220 nm): 1.93 min (LC/MS method A), calc. m/z: 395.19. |
| 248 | 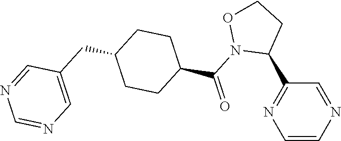 [SMILES: O=C([C@H]1CC[C@H](Cc2cncnc2)CC1)N1OCC[C@H]1c1cnccn1] | 1H NMR (400.23 MHz, DMSO-d6) d ppm 9.02 (s, 1 H), 8.60 (m, 5 H), 5.75 (s, 1 H), 5.40 (dd, J = 8.62, 6.17 Hz, 1 H), 4.30 (td, J = 7.67, 7.67, 3.48 Hz, 3 H), 4.04 (br s, 10 H), 3.94 (br dd, J = 8.44, 7.21 Hz, 5 H), 3.90 (br s, 2 H), 2.81 (m, 1 H), 2.66 (m, 1 H), 1.83 (br d, J = 12.84 Hz, 1 H), 1.73 (br s, 1 H), 1.66 (br d, J = 13.08 Hz, 2 H), 1.54 (td, J = 7.15, 7.15, 3.55 Hz, 1 H), 1.29 (m, 2 H), 1.04 (m, 2 H). LC/MS: m/z [M + H]⁺ 354.2, Rt (I = 220 nm): 1.42 min (LC/MS method A), calc. m/z: 354.19. |
| 249 | 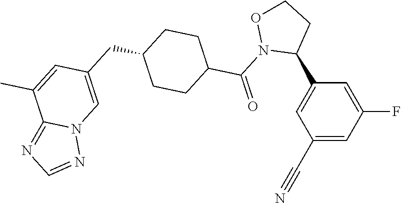 | ¹H NMR (400 MHz, DMSO-d₆): δ ppm 8.59 (s, 1 H), 8.39 (s, 1 H), 7.76 (br d, J = 7.70 Hz, 1 H), 7.59 (s, 1 H), 7.46 (br d, J = 9.54 Hz, 1 H), 7.37 (s, 1 H), 5.35 (m, 1 H), 4.25 (m, 1 H), 3.88 (m, 1 H), 2.87 (m, 1 H), 2.67 (br s, 1 H), 2.21 (m, 1 H), 1.89 (m, 1 H), 1.70 (br d, J = 9.78 Hz, 2 H), 1.59 (br s, 1 H), 1.31 (m, 2 H), 1.05 (m, 2 H). LC/MS: m/z [M + H]⁺ 448.2, Rt (I = 220 nm): 2.16 min (LC/MS method A), calc. m/z: 448.5. |
| 250 | 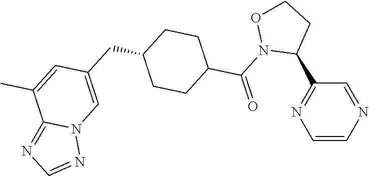 | 1H NMR (600.05 MHz, DMSO-d6) d ppm 8.58 (m, 5 H), 8.37 (s, 1 H), 7.35 (s, 1 H), 5.40 (dd, J = 8.71, 6.33 Hz, 1 H), 4.30 (td, J = 7.66, 7.66, 3.58 Hz, 1 H), 3.93 (m, 1 H), 2.81 (m, 1 H), 2.65 (m, 1 H), 1.83 (br d, J = 13.20 Hz, 2 H), 1.70 (br d, J = 12.47 Hz, 4 H), 1.57 (m, 1 H), 1.30 (m, 3 H), 1.05 (m, 3 H). LC/MS: m/z [M + H]⁺ 407.2, Rt (I = 220 nm): 1.68 min (LC/MS method A), calc. m/z: 407.49. |
| 251 | 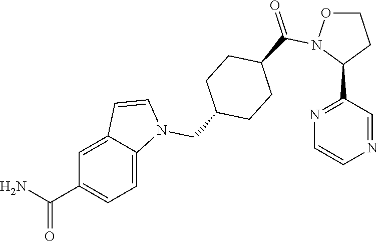 [SMILES: NC(=O)c1ccc2c(ccn2C[C@H]2CC[C@H](C(=O)N3OCC[C@H]3c3cnccn3)CC2)c1] | 1H NMR (400.23 MHz, DMSO-d6) d ppm 8.58 (m, 3 H), 8.06 (s, 1 H), 7.89 (br s, 1 H), 7.54 (m, 3 H), 7.20 (br s, 1 H), 6.47 (d, J = 3.06 Hz, 1 H), 5.39 (dd, J = 8.68, 6.24 Hz, 1 H), 4.45 (m, 3 H), 4.41 (br s, 2 H), 4.29 (td, J = 7.67, 7.67, 3.84 Hz, 4 H), 4.07 (br d, J = 7.21 Hz, 3 H), 3.93 (m, 1 H), 2.80 (m, 1 H), 2.67 (m, 1 H), 1.83 (br d, J = 11.74 Hz, 2 H), 1.72 (br d, J = 10.64 Hz, 1 H), 1.57 (br t, J = 8.13, 8.13 Hz, 2 H), 1.27 (m, 2 H), 1.12 (m, 2 H). LC/MS: m/z [M + H]⁺ 434.2, Rt (I = 220 nm): 1.7 min (LC/MS method A), calc. m/z: 434.22. |
| 252 | 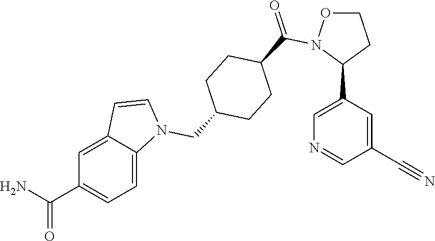 [SMILES: N#Cc1cncc([C@@H]2CCON2C(=O)[C@H]2CC[C@H](Cn3ccc4cc(C(N)=O)ccc43)CC2)c1] | 1H NMR (400.23 MHz, DMSO-d6) d ppm 8.92 (d, J = 1.96 Hz, 1 H), 8.75 (d, J = 2.08 Hz, 1 H), 8.17 (t, J = 1.90, 1.90 Hz, 1 H), 8.06 (s, 1 H), 7.88 (br s, 1 H), 7.56 (m, 2 H), 7.49 (d, J = 3.06 Hz, 1 H), 7.20 (br s, 1 H), 6.47 (d, J = 2.93 Hz, 1 H), 5.39 (m, 1 H), 4.28 (td, J = 7.70, 7.70, 2.93 Hz, 3 H), 4.07 (br d, J = 7.21 Hz, 12 H), 3.89 (m, 5 H), 3.78 (br s, 1 H), 2.88 (dddd, J = 12.17, 9.14, 6.39, 3.12 Hz, 1 H), 2.69 (m, 1 H), 2.28 (m, 1 H), 1.88 (m, 2 H), 1.72 (br d, J = 12.84 Hz, 1 H), 1.57 (m, 2 H), 1.40 (s, 1H). LC/MS: m/z [M + H]⁺ 458.2, Rt (I = 220 nm): 1.83 min (LC/MS method A), calc. m/z: 458.22. |
| 253 | 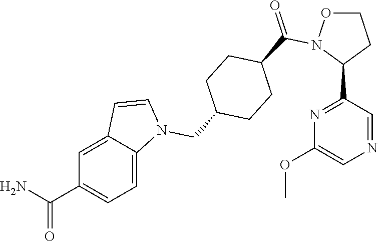 [SMILES: COc1cncc([C@@H]2CCON2C(=O)[C@H]2CC[C@H](Cn3ccc4cc(C(N)=O)ccc43)CC2)n1] | 1H NMR (400.23 MHz, DMSO-d6) d ppm 8.20 (s, 1 H), 8.10 (s, 1 H), 8.06 (s, 1 H), 7.89 (br s, 1 H), 7.56 (m, 2 H), 7.49 (d, J = 3.06 Hz, 1 H), 7.20 (br s, 1 H), 6.47 (d, J = 2.93 Hz, 1 H), 5.31 (dd, J = 8.80, 5.62 Hz, 1 H), 4.28 (td, J = 7.55, 7.55, 4.34 Hz, 2 H), 4.07 (br d, J = 7.09 Hz, 5 H), 3.94 (br dd, J = 14.98, 7.89 Hz, 4 H, under water peak), 3.88 (s, 3 H), 2.77 (m, 1 H), 2.67 (br s, 1 H), 2.43 (m, 1 H), 1.86 (br d, J = 12.10 Hz, 2 H), 1.71 (br d, J = 11.25 Hz, 1 H), 1.57 (br t, J = 9.11, 9.11 Hz, 2H), 1.27 (m, 2H), 1.11 (m, 2H). LC/MS: m/z [M + H]⁺ 464.2, Rt (I = 220 nm): 1.92 min (LC/MS method A), calc. m/z: 464.23. |
| 254 | 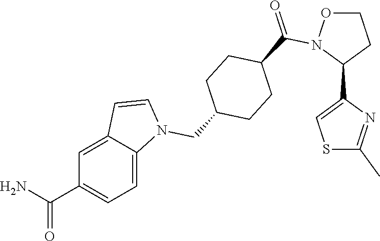 [SMILES: Cc1nc([C@@H]2CCON2C(=O)[C@H]2CC[C@H](Cn3ccc4cc(C(N)=O)ccc43)CC2)cs1] | 1H NMR (400.23 MHz, DMSO-d6) d ppm 8.06 (s, 1 H), 7.89 (br s, 1 H), 7.56 (m, 2 H), 7.48 (d, J = 3.06 Hz, 1 H), 7.18 (m, 2 H), 6.47 (d, J = 2.93 Hz, 1 H), 5.34 (br dd, J = 8.19, 5.62 Hz, 1 H), 4.22 (td, J = 7.58, 7.58, 3.79 Hz, 3 H), 4.07 (br d, J = 7.21 Hz, 3 H), 3.84 (q, J = 7.78, 7.78, 7.78 Hz, 1 H), 2.69 (m, 1 H), 2.61 (s, 4 H), 2.39 (m, 1 H), 1.82 (br d, J = 11.74 Hz, 2 H), 1.67 (br s, 1 H), 1.57 (m, 2 H), 1.28 (br t, J = 12.59, 12.59 Hz, 2 H), 1.11 (m, 2 H). LC/MS: m/z [M + H]⁺ 453.2, Rt (I = 220 nm): 1.87 min (LC/MS method A), calc. m/z: 453.20. |
| 255 | 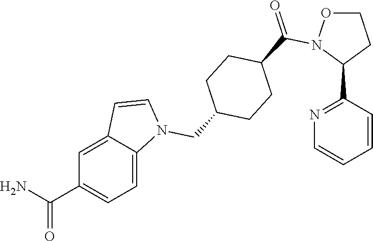 [SMILES: NC(=O)c1ccc2c(ccn2C[C@H]2CC[C@H](C(=O)N3OCC[C@H]3c3ccccn3)CC2)c1] | 1H NMR (400.23 MHz, DMSO-d6) d ppm 8.53 (d, J = 4.28 Hz, 1 H), 8.06 (s, 1 H), 7.89 (br s, 1 H), 7.81 (t, J = 7.32, 7.32 Hz, 1 H), 7.56 (m, 2 H), 7.49 (d, J = 3.06 Hz, 1 H), 7.31 (m, 2 H), 7.20 (br s, 1 H), 6.47 (d, J = 2.93 Hz, 1 H), 5.33 (dd, J = 8.80, 6.11 Hz, 1 H), 4.34 (m, 4H, under water peak), 4.24 (ddd, J = 7.58, 3.97, 3.85 Hz, 4 H), 4.07 (br d, J = 7.21 Hz, 4 H), 3.88 (m, 2 H), 2.79 (m, 1 H), 2.67 (br d, J = 1.71 Hz, 1 H), 2.42 (m, 1 H), 1.86 (br d, J = 11.74 Hz, 2 H), 1.71 (br s, 1 H), 1.57 (br t, J = 10.64 10.64, Hz, 2 H), 1.28 (m, 2H), 1.09 (m, 2H). LC/MS: m/z [M + H]⁺ 433.2, Rt (I = 220 nm): 1.73 min (LC/MS method A), calc. m/z: 433.22. |
| 256 | 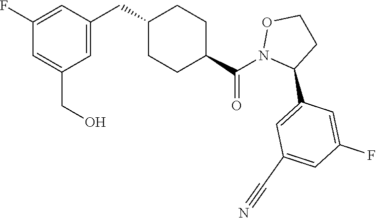 [SMILES: N#Cc1cc(F)cc([C@@H]2CCON2C(=O)[C@H]2CC[C@H](Cc3cc(F)cc(CO)c3)CC2)c1] | ¹H NMR (400 MHz, DMSO-d₆): δ ppm 7.76 (br d, J = 7.70 Hz, 1 H), 7.59 (s, 1 H), 7.46 (br d, J = 9.54 Hz, 1 H), 6.94 (m, 2 H), 6.84 (br d, J = 9.54 Hz, 1 H), 5.36 (m, 1 H), 4.47 (s, 2 H), 4.25 (td, J = 7.70, 7.70, 2.81 Hz, 1 H), 3.86 (m, 1 H), 2.87 (m, 1 H), 2.67 (m, 1 H), 2.21 (m, 1 H), 1.88 (br d, J = 12.59 Hz, 1 H), 1.67 (m, 3 H), 1.50 (br s, 1 H), 1.29 (m, 2 H), 1.01 (m, 2 H). LC/MS: m/z [M + H]⁺ 441.2, Rt (I = 220 nm): 2.37 min (LC/MS method A), calc. m/z: 441.20. |
| 257 | 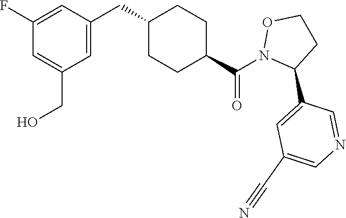 [SMILES: N#Cc1cncc([C@@H]2CCON2C(=O)[C@H]2CC[C@H](Cc3cc(F)cc(CO)c3)CC2)c1] | ¹H NMR (400 MHz, DMSO-d₆): δ ppm 8.93 (d, J = 1.96 Hz, 1 H), 8.76 (d, J = 2.08 Hz, 1 H), 8.18 (t, J = 1.90, 1.90 Hz, 1 H), 6.94 (s, 1 H), 6.84 (br d, J = 9.90 Hz, 1 H), 5.40 (m, 1 H), 4.47 (s, 2 H), 4.28 (td, J = 7.70, 7.70, 3.06 Hz, 1 H), 3.89 (m, 1 H), 2.89 (m, 1 H), 2.67 (m, 1 H), 2.29 (m, 1 H), 1.87 (br d, J = 12.84 Hz, 1 H), 1.68 (br d, J = 11.25 Hz, 3 H), 1.50 (m, 1 H), 1.29 (m, 2 H), 1.02 (m, 2 H). LC/MS: m/z [M + H]⁺ 424.2, Rt (I = 220 nm): 2.05 min (LC/MS method A), calc. m/z: 424.20. |
| 258 | 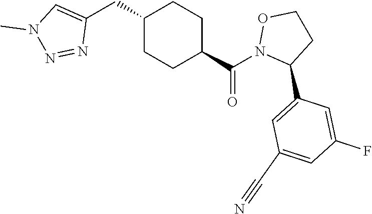 [SMILES: Cn1cc(C[C@H]2CC[C@H](C(=O)N3OCC[C@H]3c3cc(F)cc(C#N)c3)CC2)nn1] | ¹H NMR (400 MHz, DMSO-d₆): δ ppm 7.78 (s, 1 H), 7.76 (m, 1 H), 7.59 (s, 1 H), 7.46 (br d, J = 9.54 Hz, 1 H), 5.36 (dd, J = 8.62, 6.42 Hz, 1 H), 4.25 (td, J = 7.89, 7.89, 2.93 Hz, 1 H), 3.98 (s, 3 H), 3.86 (m, 1 H), 2.87 (m, 1 H), 2.66 (m, 1 H), 2.21 (m, 1 H), 1.89 (br d, J = 12.84 Hz, 1 H), 1.72 (m, 3 H), 1.51 (br s, 1 H), 1.30 (m, 2 H), 1.03 (m, 2 H). LC/MS: m/z [M + H]⁺ 398.2, Rt (I = 220 nm): 1.82 min (LC/MS method A), calc. m/z: 398.20. |
| 259 | 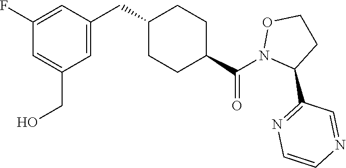 [SMILES: O=C([C@H]1CC[C@H](Cc2cc(F)cc(CO)c2)CC1)N1OCC[C@H]1c1cnccn1] | ¹H NMR (400 MHz, DMSO-d₆): δ ppm 8.58 (m, 3 H), 6.93 (m, 3 H), 6.84 (br d, J = 9.78 Hz, 1 H), 5.75 (s, 1 H), 5.40 (m, 1 H), 4.47 (s, 2 H), 4.30 (td, J = 7.64, 7.64, 3.55 Hz, 1 H), 3.93 (m, 1 H), 2.81 (m, 1 H), 2.66 (m, 1 H), 1.82 (br d, J = 11.62 Hz, 1 H), 1.67 (br d, J = 12.84 Hz, 3 H), 1.50 (m, 1 H), 1.28 (m, 2 H), 1.01 (m, 2 H). LC/MS: m/z [M + H]⁺ 400.2, Rt (I = 220 nm): 1.92 min (LC/MS method A), calc. m/z: 400.20. |
| 260 | 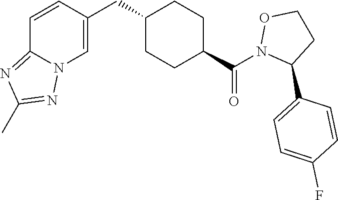 [SMILES: Cc1nc2ccc(C[C@H]3CC[C@H](C(=O)N4OCC[C@H]4c4ccc(F)cc4)CC3)cn2n1] | ¹H NMR (400 MHz, DMSO-d₆): δ ppm 8.63 (s, 1 H), 7.61 (d, J = 9.05 Hz, 1 H), 7.47 (dd, J = 9.05, 1.59 Hz, 1 H), 7.29 (dd, J = 8.62, 5.56 Hz, 2 H), 7.15 (t, J = 8.31, 8.31 Hz, 2 H), 5.29 (m, 1 H), 4.23 (td, J = 7.70, 7.70, 2.93 Hz, 1 H), 3.83 (m, 1 H), 2.84 (m, 1 H), 2.67 (br d, J = 1.96 Hz, 1 H), 2.56 (m, 2 H), 2.44 (s, 3 H), 2.15 (m, 1 H), 1.85 (br d, J = 12.59 Hz, 1 H), 1.69 (br d, J = 12.47 Hz, 3 H), 1.56 (br dd, J = 6.85, 3.79 Hz, 1 H), 1.30 (m, 2 H), 1.04 (m, 2 H). LC/MS: m/z [M + H]⁺ 423.2, Rt (I = 220 nm): 2.07 min (LC/MS method A), calc. m/z: 423.22. |
| 261 | 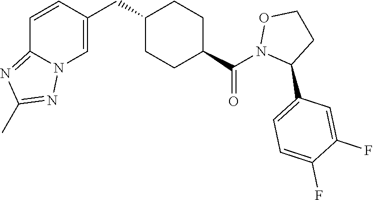 [SMILES: Cc1nc2ccc(C[C@H]3CC[C@H](C(=O)N4OCC[C@H]4c4ccc(F)c(F)c4)CC3)cn2n1] | 1H NMR (400.23 MHz, DMSO-d6) d ppm 8.66 (s, 1 H), 7.64 (d, J = 9.17 Hz, 1 H), 7.51 (d, J = 9.05 Hz, 1 H), 7.39 (m, 1 H), 7.28 (m, 1 H), 7.11 (br s, 1 H), 5.29 (br t, J = 7.46, 7.46 Hz, 1 H), 4.23 (td, J = 7.67, 7.67, 3.00 Hz, 1 H), 3.83 (m, 4 H), 2.84 (m, 1 H), 2.67 (br s, 1 H), 2.57 (s, 2 H), 2.17 (m, 1 H), 1.86 (br d, J = 11.98 Hz, 1 H), 1.69 (br d, J = 9.29 Hz, 3 H), 1.57 (br s, 1 H), 1.30 (m, 2 H), 1.04 (m, 2 H). LC/MS: m/z [M + H]⁺ 441.2, Rt (I = 220 nm): 2.15 min (LC/MS method A), calc. m/z: 441.21. |
| 262 | 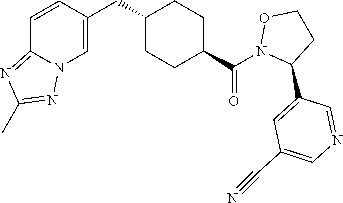 [SMILES: Cc1nc2ccc(C[C@H]3CC[C@H](C(=O)N4OCC[C@H]4c4cncc(C#N)c4)CC3)cn2n1] | 1H NMR (400.23 MHz, DMSO-d6) d ppm 8.93 (d, J = 1.96 Hz, 1 H), 8.76 (d, J = 2.08 Hz, 1 H), 8.67 (s, 1 H), 8.18 (t, J = 1.90, 1.90 Hz, 1 H), 7.65 (d, J = 9.05 Hz, 1 H), 7.53 (dd, J = 9.17, 1.34 Hz, 1 H), 5.39 (dd, J = 8.50, 6.54 Hz, 1 H), 4.28 (td, J = 7.73, 7.73, 3.00 Hz, 4 H, under water peak), 3.89 (m, 1 H), 2.89 (dddd, J = 12.10, 9.14, 6.45, 3.12 Hz, 1 H), 2.59 (m, 3 H), 2.28 (m, 1 H), 1.88 (br d, J = 11.86 Hz, 1 H), 1.69 (br d, J = 11.37 Hz, 3 H), 1.57 (br dd, J = 7.03, 3.85 Hz, 1 H), 1.29 (m, 2 H), 1.05 (m, 2 H). LC/MS: m/z [M + H]⁺ 431.3, Rt = (I = 220 nm): 1.7 min (LC/MS method A), calc. m/z: 431.22. |
| 263 | 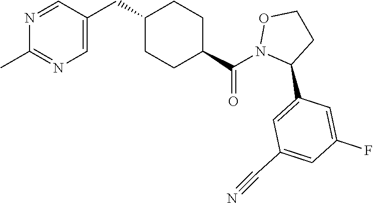 [SMILES: Cc1ncc(C[C@H]2CC[C@H](C(=O)N3OCC[C@H]3c3cc(F)cc(C#N)c3)CC2)cn1] | 1H NMR (400.23 MHz, DMSO-d6) d ppm 8.52 (s, 2 H), 7.76 (d, J = 7.88 Hz, 1 H), 7.59 (s, 1 H), 7.46 (br d, J = 9.66 Hz, 1 H), 5.35 (dd, J = 8.56, 6.36 Hz, 1 H), 4.25 (td, J = 7.76, 7.76, 2.93 Hz, 1 H), 3.89 (br s, 2 H), 3.85 (br dd, J = 8.13, 6.79 Hz, 4 H, under water peak), 2.87 (dddd, J = 12.18, 9.19, 6.51, 3.06 Hz, 1 H), 2.66 (m, 1 H), 2.57 (s, 3 H), 2.45 (s, 1 H), 2.21 (m, 1 H), 1.88 (br d, J = 12.59 Hz, 1 H), 1.66 (br d, J = 12.47 Hz, 3 H), 1.51 (m, 1 H), 1.30 (q, J = 12.55, 12.55, 12.55 Hz, 2 H), 1.03 (m, 2 H). LC/MS: m/z [M + H]⁺ 409.2, Rt (I = 220 nm): 1.97 min (LC/MS method A), calc. m/z: 409.20. |
| 264 | 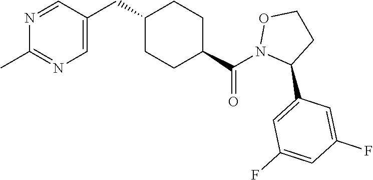 [SMILES: Cc1ncc(C[C@H]2CC[C@H](C(=O)N3OCC[C@H]3c3cc(F)cc(F)c3)CC2)cn1] | 1H NMR (400.23 MHz, DMSO-d6) d ppm 8.51 (s, 2 H), 7.12 (t, J = 9.25, 9.25 Hz, 1 H), 6.96 (br d, J = 6.48 Hz, 2 H), 5.32 (m, 1 H), 4.23 (m, 1 H), 3.84 (m, 1 H), 2.85 (m, 1 H), 2.67 (br s, 1 H), 2.57 (s, 3 H), 2.45 (s, 1 H), 2.17 (m, 1 H), 1.88 (br d, J = 13.08 Hz, 1 H), 1.67 (br s, 3 H), 1.51 (br s, 1 H), 1.29 (m, 2 H), 1.01 (m, 2 H). LC/MS: m/z [M + H]⁺ 402.2, Rt (I = 220 nm): 2.12 min (LC/MS method A), calc. m/z: 402.20. |
| 265 | 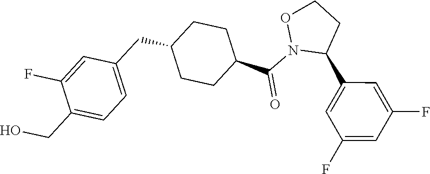 [SMILES: O=C([C@H]1CC[C@H](Cc2ccc(CO)c(F)c2)CC1)N1OCC[C@H]1c1cc(F)cc(F)c1] | ¹H NMR (400 MHz, DMSO-d₆): δ ppm 7.34 (t, J = 7.76, 7.76 Hz, 1 H), 7.12 (tt, J = 9.34, 9.34, 2.28, 2.28 Hz, 1 H), 6.96 (m, 4 H), 5.31 (m, 1 H), 4.49 (s, 2 H), 4.23 (td, J = 7.79, 7.79, 2.87 Hz, 1 H), 3.84 (m, 1 H), 2.85 (m, 1 H), 2.67 (br d, J = 1.59 Hz, 1 H), 2.17 (m, 1 H), 1.87 (br d, J = 13.20 Hz, 1 H), 1.67 (br d, J = 8.19 Hz, 3 H), 1.49 (br s, 1 H), 1.29 (m, 2 H), 1.01 (m, 2 H). LC/MS: m/z [M + H]⁺ 434.2, Rt (I = 220 nm): 2.48 min (LC/MS method A), calc. m/z: 434.19. |
| 266 | 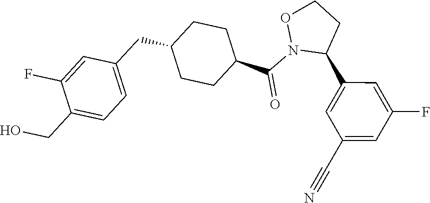 [SMILES: N#Cc1cc(F)cc([C@@H]2CCON2C(=O)[C@H]2CC[C@H](Cc3ccc(CO)c(F)c3)CC2)c1] | ¹H NMR (400 MHz, DMSO-d₆): δ ppm 7.77 (m, 1 H), 7.59 (s, 1 H), 7.46 (br d, J = 9.41 Hz, 1 H), 7.34 (t, J = 7.82, 7.82 Hz, 1 H), 6.95 (m, 2 H), 5.35 (dd, J = 8.62, 6.54 Hz, 1 H), 4.49 (s, 2 H), 4.25 (td, J = 7.82, 7.82, 2.93 Hz, 1 H), 3.85 (m, 1 H), 2.87 (m, 1 H), 2.66 (m, 1 H), 2.21 (m, 1 H), 1.87 (br d, J = 12.59 Hz, 1 H), 1.68 (m, 3 H), 1.50 (br d, J = 3.55 Hz, 1 H), 1.29 (m, 2 H), 1.00 (m, 2 H). LC/MS: m/z [M + H]⁺ 441.2, Rt (I = 220 nm): 2.34 min (LC/MS method A), calc. m/z: 441.20. |
| 267 | 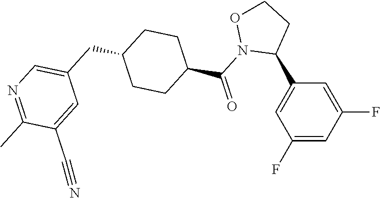 [SMILES: Cc1ncc(C[C@H]2CC[C@H](C(=O)N3OCC[C@H]3c3cc(F)cc(F)c3)CC2)cc1C#N] | ¹H NMR (400 MHz, MeOD): δ ppm 8.48 (d, J = 2.1 Hz, 1 H), 7.96 (d, J = 2.0 Hz, 1 H), 6.89 (br d, J = 6.6 Hz, 2 H), 6.83 (dt, J = 2.3, 9.1 Hz, 1 H), 5.33 (br t, J = 7.2 Hz, 1 H), 4.28 (dt, J = 3.2, 7.8 Hz, 1 H), 3.94 (m, 1 H), 2.92 (m, 1 H), 2.86-2.73 (m, 1 H), 2.70 (s, 3 H), 2.59 (d, J = 7.1 Hz, 2 H), 2.33-2.23 (m, 1 H), 1.97 (br d, J = 12.1 Hz, 1 H), 1.81-1.70 (m, 3 H), 1.59 (m, 1 H), 1.47-1.38 (m, 2 H), 1.19-1.02 (m, 2 H). LC/MS: m/z [M + H]⁺ 426.3, Rt (I = 220 nm): 0.94 min (LC/MS method B), calc. m/z: 426.20. |
| 268 | 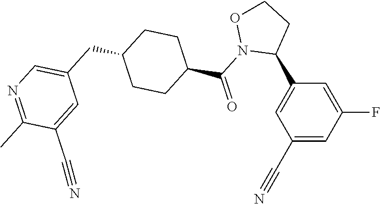 [SMILES: Cc1ncc(C[C@H]2CC[C@H](C(=O)N3OCC[C@H]3c3cc(F)cc(C#N)c3)CC2)cc1C#N] | ¹H NMR (400 MHz, MeOD): δ ppm 8.48 (d, J = 2.0 Hz, 1 H), 7.96 (d, J = 2.0 Hz, 1 H), 7.50 (s, 1 H), 7.46 (br d, J = 8.1 Hz, 1 H), 7.38 (br d, J = 9.3 Hz, 1 H), 5.37 (br t, J = 7.6 Hz, 1 H), 4.31 (dt, J = 3.0, 7.8 Hz, 1 H), 3.95 (m, 1 H), 2.95 (m, 1 H), 2.81 (br t, J = 14.8 Hz, 1 H), 2.70 (s, 3 H), 2.59 (d, J = 7.1 Hz, 2 H), 2.34-2.25 (m, 1 H), 2.00-1.94 (m, 1 H), 1.77 (br d, J = 7.6 Hz, 3 H), 1.63- 1.54 (m, 1 H), 1.47-1.37 (m, 2 H), 1.19-1.06 (m, 2 H). LC/MS: m/z [M + H]⁺ 433.3, Rt (I = 220 nm): 0.92 min (LC/MS method B), calc. m/z: 433.20. |
| 269 | 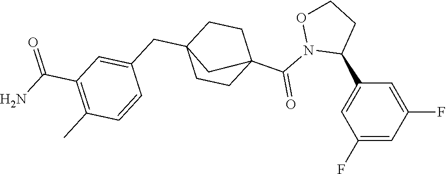 [SMILES: Cc1ccc(CC23CCC(C(=O)N4OCC[C@H]4c4cc(F)cc(F)c4)(CC2)C3)cc1C(N)=O] | 1H NMR (400.23 MHz, DMSO-d6) d ppm 7.61 (br s, 1 H), 7.29 (br s, 1 H), 7.10 (m, 4 H), 6.95 (m, 2 H), 5.33 (dd, J = 8.56, 6.72 Hz, 1 H), 4.22 (td, J = 7.64, 7.64, 2.32 Hz, 1 H), 3.74 (m, 1 H), 2.85 (m, 1 H), 2.73 (s, 2 H), 2.32 (s, 3 H), 2.13 (m, 1 H), 1.79 (m, 4 H), 1.51 (m, 4 H), 1.28 (br s, 2 H). LC/MS: m/z [M + H]⁺ 455.2, Rt (I = 220 nm): 2.39 min (LC/MS method A), calc. m/z: 455.22. |
| 270 | 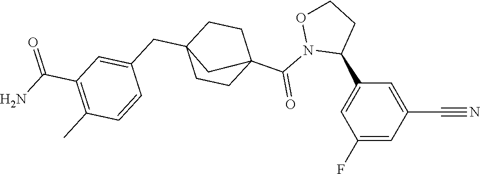 [SMILES: Cc1ccc(CC23CCC(C(=O)N4OCC[C@H]4c4cc(F)cc(C#N)c4)(CC2)C3)cc1C(N)=O] | 1H NMR (400.23 MHz, DMSO-d6) d ppm 7.76 (dd, J = 2.32, 1.34 Hz, 1 H), 7.74 (s, 1 H), 7.58 (m, 2 H), 7.44 (br d, J = 9.41 Hz, 1 H), 7.29 (br s, 1 H), 7.10 (m, 3 H), 5.37 (m, 1 H), 4.24 (td, J = 7.67, 7.67, 2.14 Hz, 1 H), 3.77 (m, 1 H), 2.87 (dtd, J = 12.15, 6.06, 6.06, 3.12 Hz, 1 H), 2.73 (s, 2 H), 2.32 (s, 3 H), 2.18 (m, 1 H), 1.84 (br s, 1 H), 1.79 (td, J = 8.10, 8.10, 4.46 Hz, 3 H), 1.51 (m, 4 H), 1.28 (br s, 2 H). LC/MS: m/z [M + H]⁺ 462.2, Rt (I = 220 nm): 2.25 min (LC/MS method A), calc. m/z: 462.22. |
| 271 | 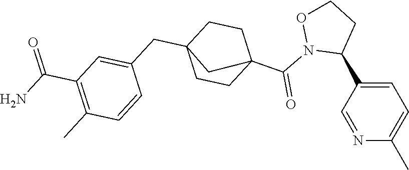 [SMILES: Cc1ccc([C@@H]2CCON2C(=O)C23CCC(Cc4ccc(C)c(C(N)=O)c4)(CC2)C3)cn1] | 1H NMR (400.23 MHz, DMSO-d6) d ppm 8.36 (d, J = 2.20 Hz, 1 H), 7.60 (m, 2 H), 7.28 (m, 2 H), 7.10 (m, 3 H), 5.32 (m, 1 H), 4.25 (td, J = 7.70, 7.70, 2.57 Hz, 1 H), 3.77 (m, 1 H), 2.84 (m, 1 H), 2.73 (s, 2 H), 2.67 (m, 1 H), 2.32 (s, 3 H), 2.17 (m, 1 H), 1.77 (br d, J = 6.36 Hz, 4 H), 1.50 (m, 4 H), 1.26 (br s, 2 H). LC/MS: m/z [M + H]⁺ 434.2, Rt (I = 220 nm): 1.46 min (LC/MS method A), calc. m/z: 434.25. |
| 272 | 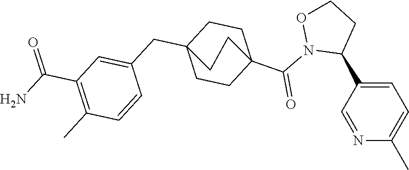 [SMILES: Cc1ccc([C@@H]2CCON2C(=O)C23CCC(Cc4ccc(C)c(C(N)=O)c4)(CC2)CC3)cn1] | 1H NMR (400.23 MHz, DMSO-d6) d ppm 8.31 (d, J = 2.08 Hz, 1 H), 7.61 (br s, 1 H), 7.52 (dd, J = 7.95, 1.96 Hz, 1 H), 7.29 (br s, 1 H), 7.22 (d, J = 8.07 Hz, 1 H), 7.10 (d, J = 7.70 Hz, 1 H), 7.03 (s, 1 H), 7.00 (d, J = 7.51 Hz, 1 H), 5.27 (m, 1 H), 4.25 (td, J = 7.79, 7.79, 2.26 Hz, 1 H), 3.80 (m, 1 H), 2.81 (m, 1 H), 2.43 (s, 3 H), 2.38 (s, 2 H), 2.31 (s, 3 H), 2.12 (m, 1 H), 1.78 (br s, 2 H), 1.75 (br d, J = 8.56 Hz, 4 H), 1.37 (br d, J = 8.19 Hz, 4 H), 1.35 (br s, 2 H). LC/MS: m/z [M + H]⁺ 448.3, Rt (I = 220 nm): 1.51 min (LC/MS method A), calc. m/z: 448.26. |
| 273 | 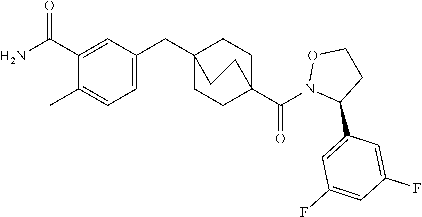 [SMILES: Cc1ccc(CC23CCC(C(=O)N4OCC[C@H]4c4cc(F)cc(F)c4)(CC2)CC3)cc1C(N)=O] | 1H NMR (400 MHz, DMSO-d6) δ ppm 7.63 (br s, 1H), 7.32 (br s, 1H), 7.14-7.07 (m, 2H), 7.05-6.98 (m, 2H), 6.90 (br d, J = 6.6 Hz, 2H), 5.29 (t, J = 7.8 Hz, 1H), 4.26-4.19 (m, 1H), 3.81-3.73 (m, 1H), 2.86- 2.78 (m, 1H), 2.38 (s, 2H), 2.31 (s, 3H), 2.15-2.05 (m, 1H), 1.81-1.74 (m, 6H), 1.40-1.34 (m, 6H). LC/MS: m/z [M + H]⁺ 469.3, Rt (I = 220 nm): 0.97 min (LC/MS method B), calc. m/z: 469.54. |
| 274 | 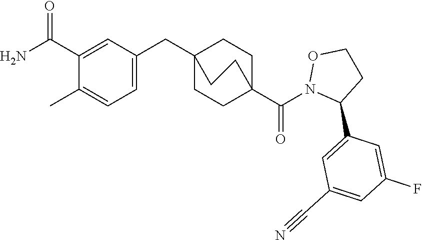 [SMILES: Cc1ccc(CC23CCC(C(=O)N4OCC[C@H]4c4cc(F)cc(C#N)c4)(CC2)CC3)cc1C(N)=O] | 1H NMR (400 MHz, DMSO-d6): δ ppm 7.75 (br d, J = 8.3 Hz, 1H), 7.63 (br s, 1H), 7.53 (s, 1H), 7.39 (br d, J = 9.5 Hz, 1H), 7.32 (br s, 1H), 7.11 (d, J = 7.7 Hz, 1H), 7.04-6.99 (m, 2H), 5.33 (t, J = 7.8 Hz, 1H), 4.28- 4.21 (m, 1H), 3.83-3.75 (m, 1H), 2.88-2.79 (m, 1H), 2.38 (s, 2H), 2.31 (s, 3H), 2.14 (tdd, J = 7.4, 9.8, 12.0 Hz, 1H), 1.81-1.74 (m, 6H), 1.40-1.34 (m, 6H). LC/MS: m/z [M + H]⁺ 476.3, Rt (I = 220 nm): 0.95 min (LC/MS method B), calc. m/z: 476.24. |
| 275 | 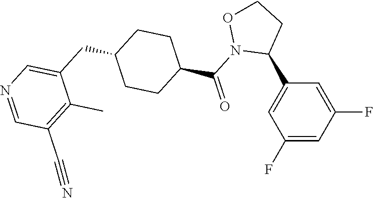 [SMILES: Cc1c(C#N)cncc1C[C@H]1CC[C@H](C(=O)N2OCC[C@H]2c2cc(F)cc(F)c2)CC1] | 1H NMR (400 MHz, CDCl3) δ ppm 8.68 (br s, 1H), 8.49 (br s, 1H), 6.88-6.77 (m, 2H), 6.75-6.66 (m, 1H), 5.37 (dd, J = 8.7, 6.1 Hz, 1H), 4.27 (td, J = 7.8, 3.5 Hz, 1H), 3.89 (q, J = 8.3 Hz, 1H), 2.89-2.73 (m, 2H), 2.63 (dd, J = 7.0, 3.4 Hz, 2H), 2.57 (s, 3H), 2.39-2.25 (m, 1H), 2.04 (br d, J = 13.4 Hz, 1H), 1.83-1.74 (m, 3H), 1.64-1.55 (m, 1H), 1.54-1.44 (m, 2H), 1.27-1.09 (m, 2H). LC/MS: m/z [M + H]⁺ 426.3, Rt (I = 220 nm): 0.86 min (LC/MS method B), calc. m/z: 426.20. |
| 276 | 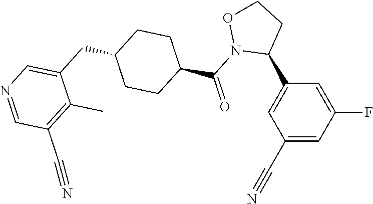 [SMILES: Cc1c(C#N)cncc1C[C@H]1CC[C@H](C(=O)N2OCC[C@H]2c2cc(F)cc(C#N)c2)CC1] | 1H NMR (400 MHz, CDCl3) δ = 8.60 (s, 1H), 8.40 (s, 1H), 7.31 (s, 1H), 7.17 (s, 2H), 5.31 (dd, J = 8.6, 6.2 Hz, 1H), 4.21 (td, J = 7.9, 2.6 Hz, 1H), 3.87-3.76 (m, 1H), 2.86-2.76 (m, 1H), 2.75-2.64 (m, 1H), 2.55 (br t, J = 6.3 Hz, 2H), 2.48 (s, 3H), 2.28-2.16 (m, 1H), 1.98- 1.92 (m, 1H), 1.75-1.67 (m, 3H), 1.53-1.45 (m, 1H), 1.44 (br s, 2H), 1.17-1.00 (m, 2H). LC/MS: m/z [M + H]⁺ 433.2, Rt (I = 220 nm): 0.84 min (LC/MS method B), calc. m/z: 433.22. |
| 277 | 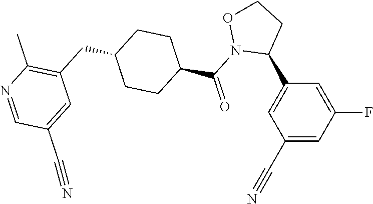 [SMILES: Cc1ncc(C#N)cc1C[C@H]1CC[C@H](C(=O)N2OCC[C@H]2c2cc(F)cc(C#N)c2)CC1] | ¹H NMR (400 MHz, DMSO-d₆): δ ppm 8.72 (d, J = 2.0 Hz, 1 H), 8.01 (d, J = 1.9 Hz, 1 H), 7.76 (br d, J = 8.1 Hz, 1 H), 7.59 (s, 1 H), 7.46 (br d, J = 9.6 Hz, 1 H), 5.36 (dd, J = 6.9, 8.3 Hz, 1 H), 4.26 (dt, J = 2.8, 7.7 Hz, 1 H), 3.89-3.83 (m, 1 H), 2.88 (m, 1 H), 2.74- 2.64 (m, 1 H), 2.56 (br s, 1 H), 2.55 (s, 3 H), 2.25- 2.18 (m, 1 H), 1.88 (br d, J = 12.1 Hz, 1 H), 1.72- 1.52 (m, 5 H), 1.35-1.26 (m, 2 H), 1.15-1.06 (m, 2 H). LC/MS: m/z [M + H]⁺ 433.1, Rt (I = 220 nm): 0.93 min (LC/MS method B), calc. m/z: 433.21. |
| 278 | 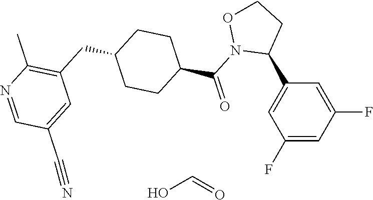 [SMILES: Cc1ncc(C#N)cc1C[C@H]1CC[C@H](C(=O)N2OCC[C@H]2c2cc(F)cc(F)c2)CC1 ; O=CO] | ¹H NMR (400 MHz, DMSO-d₆): δ ppm 8.72 (d, J = 2.0 Hz, 1 H), 8.01 (d, J = 2.0 Hz, 1 H), 7.13 (tt, J = 2.3, 9.3 Hz, 1 H), 6.96 (br d, J = 6.6 Hz, 2 H), 5.32 (dd, J = 6.6, 8.2 Hz, 1 H), 4.24 (dt, J = 2.8, 7.7 Hz, 1 H), 3.87-3.81 (m, 1 H), 2.86 (m, 1 H), 2.74-2.64 (m, 1 H), 2.56-2.54 (m, 5 H), 2.22-2.13 (m, 1 H), 1.91-1.84 (m, 1 H), 1.69- 1.61 (m, 3 H), 1.56 (m, 1 H), 1.36-1.27 (m, 2 H), 1.16- 1.05 (m, 2 H). LC/MS: m/z [M + H]⁺ 426, Rt (I = 220 nm): 0.96 min (LC/MS method B), calc. m/z: 426.20. |
| 279 | 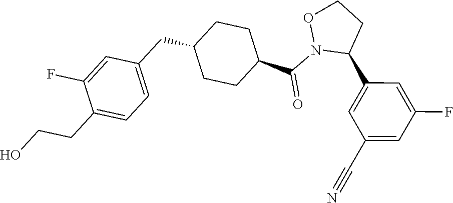 [SMILES: N#Cc1cc(F)cc([C@@H]2CCON2C(=O)[C@H]2CC[C@H](Cc3ccc(CCO)c(F)c3)CC2)c1] | 1H NMR (400.23 MHz, DMSO-d6) d ppm 7.76 (d, J = 7.98 Hz, 1 H), 7.59 (s, 1 H), 7.46 (br d, J = 9.66 Hz, 1 H), 7.19 (t, J = 8.07, 8.07 Hz, 1 H), 6.91 (m, 2 H), 5.35 (dd, J = 8.44, 6.48 Hz, 1 H), 4.25 (td, J = 7.67, 7.67, 3.00 Hz, 1 H), 3.85 (m, 1 H), 2.87 (dddd, J = 12.17, 9.14, 6.45, 3.06 Hz, 1 H), 2.71 (br t, J = 7.03, 7.03 Hz, 2 H), 2.67 (m, 1 H), 2.44 (s, 1 H), 2.21 (m, 1 H), 1.87 (br d, J = 12.59 Hz, 1 H), 1.68 (m, 3 H), 1.49 (br d, J = 3.30 Hz, 1 H), 1.29 (m, 2 H), 1.00 (br t, J = 12.72, 12.72 Hz, 2 H). LC/MS: m/z [M + H]⁺ 455.2, Rt (I = 220 nm): 2.42 min (LC/MS method A), calc. m/z: 455.22. |
| 280 | 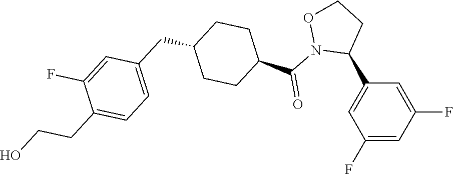 [SMILES: O=C([C@H]1CC[C@H](Cc2ccc(CCO)c(F)c2)CC1)N1OCC[C@H]1c1cc(F)cc(F)c1] | 1H NMR (400.23 MHz, DMSO-d6) d ppm 7.19 (t, J = 8.07, 8.07 Hz, 1 H), 7.12 (tt, J = 9.29, 9.29, 2.32, 2.32 Hz, 1 H), 6.93 (m, 4 H), 5.31 (dd, J = 8.68, 6.36 Hz, 1 H), 4.67 (t, J = 5.32, 5.32 Hz, 1 H), 4.23 (td, J = 7.73, 7.73, 3.00 Hz, 1 H), 3.84 (m, 1 H), 3.57 (m, 2 H), 2.85 (dddd, J = 12.07, 9.11, 6.45, 3.06 Hz, 1 H), 2.71 (t, J = 7.03, 7.03 Hz, 2 H), 2.67 (m, 1 H), 2.44 (s, 1 H), 2.17 (m, 1 H), 1.87 (br d, J = 12.59 Hz, 1 H), 1.67 (m, 3 H), 1.49 (br s, 1 H), 1.29 (q, J = 12.63, 12.63, 12.63 Hz, 2 H), 1.01 (m, 2 H). LC/MS: m/z [M + H]⁺ 448.2, Rt (I = 220 nm): 2.55 min (LC/MS method A), calc. m/z: 448.21. |
| 281 | 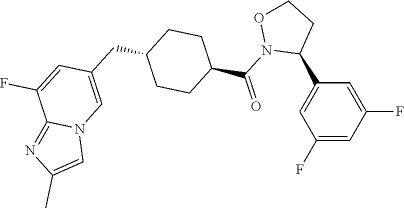 [SMILES: Cc1cn2cc(C[C@H]3CC[C@H](C(=O)N4OCC[C@H]4c4cc(F)cc(F)c4)CC3)cc(F)c2n1] | ¹H NMR (400 MHz, DMSO-d₆): δ ppm 8.39 (s, 1 H), 7.97 (br s, 1 H), 7.55 (br s, 1 H), 7.12 (t, J = 9.37, 9.37 Hz, 1 H), 6.96 (br d, J = 6.60 Hz, 2 H), 5.31 (m, 1 H), 4.23 (td, J = 7.73, 7.73, 3.00 Hz, 1 H), 3.85 (m, 1 H), 2.86 (m, 1 H), 2.67 (m, 1 H), 2.43 (s, 3 H), 2.18 (m, 1 H), 1.89 (br d, J = 13.33 Hz, 1 H), 1.70 (br d, J = 8.68 Hz, 3 H), 1.59 (br s, 1 H), 1.32 (m, 2 H), 1.05 (m, 2 H). LC/MS: m/z [M + H]⁺ 458.2, Rt (I = 220 nm): 1.73 min (LC/MS method A), calc. m/z: 458.21. |
| 282 | 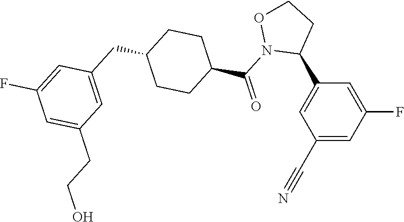 [SMILES: N#Cc1cc(F)cc([C@@H]2CCON2C(=O)[C@H]2CC[C@H](Cc3cc(F)cc(CCO)c3)CC2)c1] | 1H NMR (400.23 MHz, DMSO-d6) d ppm 7.76 (d, J = 7.80 Hz, 1 H), 7.59 (s, 1 H), 7.46 (br d, J = 9.66 Hz, 1 H), 6.84 (s, 2 H), 6.82 (m, 1 H), 5.36 (dd, J = 8.44, 6.60 Hz, 1 H), 4.62 (br s, 1 H), 4.25 (td, J = 7.67, 7.67, 2.87 Hz, 1 H), 3.86 (m, 1 H), 3.60 (t, J = 6.91, 6.91 Hz, 2 H), 2.87 (dddd, J = 12.15, 9.15, 6.45, 2.93 Hz, 1 H), 2.70 (br t, J = 6.91, 6.91 Hz, 3 H), 2.44 (s, 1 H), 2.21 (m, 1 H), 1.88 (br d, J = 12.59 Hz, 1 H), 1.67 (br s, 3 H), 1.49 (br s, 1 H), 1.30 (m, 2 H), 1.01 (br t, J = 12.65, 12.65 Hz, 2 H). LC/MS: m/z [M + H]⁺ 455.2, Rt (I = 220 nm): 2.42 min (LC/MS method A), calc. m/z: 455.22. |
| 283 | 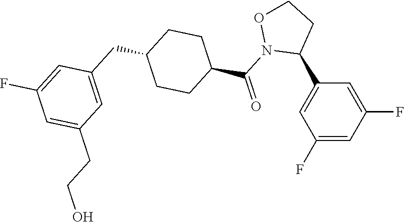 [SMILES: O=C([C@H]1CC[C@H](Cc2cc(F)cc(CCO)c2)CC1)N1OCC[C@H]1c1cc(F)cc(F)c1] | 1H NMR (400.23 MHz, DMSO-d6) d ppm 7.12 (tt, J = 9.35, 9.35, 2.32, 2.32 Hz, 1 H), 6.96 (br d, J = 6.60 Hz, 2 H), 6.84 (m, 3 H), 5.31 (m, 1 H), 4.62 (t, J = 6.48, 6.48 Hz, 1 H), 4.23 (td, J = 7.61, 7.61, 2.87 Hz, 1 H), 3.83 (m, 1 H), 3.60 (t, J = 6.91, 6.91 Hz, 2 H), 3.01 (t, J = 6.48, 6.48 Hz, 1 H), 2.85 (dddd, J = 12.09, 9.06, 6.30, 2.75 Hz, 1 H), 2.70 (br t, J = 6.85, 6.85 Hz, 2 H), 2.68 (m, 1 H), 2.44 (br s, 1 H), 2.17 (m, 1 H), 1.87 (br d, J = 11.49 Hz, 1 H), 1.68 (br d, J = 8.80 Hz, 3 H), 1.50 (br s, 1 H), 1.29 (m, 2H), 1.01 (m, 2H). LC/MS: m/z [M + H]⁺ 448.2, Rt (I = 220 nm): 2.56 min (LC/MS method A), calc. m/z: 448.21. |
| 284 | 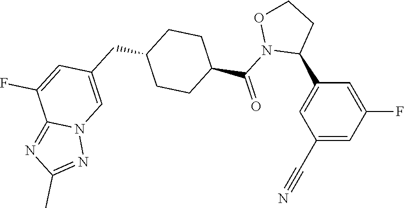 [SMILES: Cc1nc2c(F)cc(C[C@H]3CC[C@H](C(=O)N4OCC[C@H]4c4cc(F)cc(C#N)c4)CC3)cn2n1] | 1H NMR (400.23 MHz, DMSO-d6) d ppm 8.58 (s, 1 H), 7.76 (br d, J = 7.46 Hz, 1 H), 7.59 (s, 1 H), 7.49 (d, J = 11.49 Hz, 1 H), 7.46 (br d, J = 9.54 Hz, 1 H), 5.36 (m, 1 H), 4.25 (td, J = 7.79, 7.79, 3.00 Hz, 1 H), 3.83 (m, 14 H), 2.87 (m, 1 H), 2.68 (m, 1 H), 2.56 (s, 1 H), 2.33 (s, 1 H), 2.21 (m, 1 H), 1.89 (br d, J = 11.98 Hz, 1 H), 1.70 (br d, J = 11.49 Hz, 3 H), 1.60 (br s, 1 H), 1.31 (m, 2 H), 1.04 (m, 2 H). LC/MS: m/z [M + H]⁺ 466.2, Rt (I = 220 nm): 2.19 min (LC/MS method A), calc. m/z: 466.21. |
| 285 | 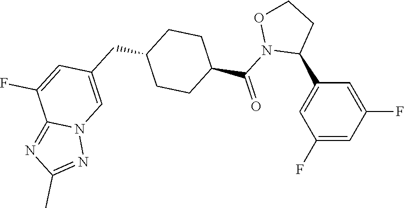 [SMILES: Cc1nc2c(F)cc(C[C@H]3CC[C@H](C(=O)N4OCC[C@H]4c4cc(F)cc(F)c4)CC3)cn2n1] | 1H NMR (400.23 MHz, DMSO-d6) d ppm 8.58 (s, 1 H), 7.50 (d, J = 11.37 Hz, 1 H), 7.12 (m, 1 H), 6.96 (br d, J = 6.48 Hz, 2 H), 5.32 (m, 1 H), 4.23 (m, 1 H), 3.84 (m, 1 H), 2.85 (ddd, J = 12.07, 5.84, 3.48 Hz, 1 H), 2.67 (br s, 1 H), 2.56 (s, 1 H), 2.17 (m, 1 H), 1.88 (br d, J = 11.86 Hz, 1 H), 1.69 (br d, J = 10.15 Hz, 3 H), 1.60 (br s, 1 H), 1.31 (q, J = 12.47, 12.47, 12.47 Hz, 2 H), 1.04 (m, 2 H). LC/MS: m/z [M + H]⁺ 459.2, Rt (I = 220 nm): 2.33 min (LC/MS method A), calc. m/z: 459.20. |
| 286 | 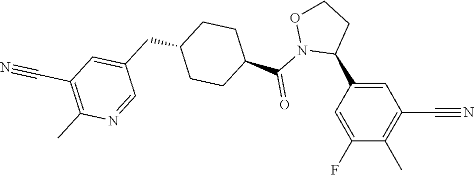 [SMILES: Cc1ncc(C[C@H]2CC[C@H](C(=O)N3OCC[C@H]3c3cc(F)c(C)c(C#N)c3)CC2)cc1C#N] | ¹H NMR (400 MHz, DMSO-d₆): δ ppm 8.54 (d, J = 2.08 Hz, 1 H), 8.06 (d, J = 2.08 Hz, 1 H), 7.52 (s, 1 H), 7.40 (d, J = 10.39 Hz, 1 H), 5.31 (dd, J = 8.44, 6.48 Hz, 1 H), 4.24 (td, J = 7.70, 7.70, 3.06 Hz, 1 H), 3.85 (m, 1 H), 2.85 (m, 1 H), 2.63 (m, 1 H), 2.61 (s, 3 H), 2.50 (m, 2 H), 2.37 (d, J = 1.83 Hz, 3 H), 2.20 (m, 1 H), 1.86 (br d, J = 12.35 Hz, 1 H), 1.66 (m, 3 H), 1.54 (br d, J = 3.55 Hz, 1 H), 1.28 (m, 2 H), 1.02 (m, 2 H). LC/MS: m/z [M + H]⁺ 447.3, Rt (I = 220 nm): 2.43 min (LC/MS method A), calc. m/z: 447.22. |
| 287 | 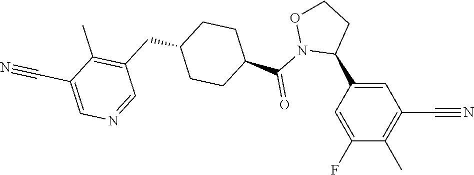 [SMILES: Cc1c(F)cc([C@@H]2CCON2C(=O)[C@H]2CC[C@H](Cc3cncc(C#N)c3C)CC2)cc1C#N] | ¹H NMR (400 MHz, DMSO-d₆): δ ppm 8.76 (s, 1 H), 8.54 (s, 1 H), 7.52 (s, 1 H), 7.40 (d, J = 10.27 Hz, 1 H), 5.31 (m, 1 H), 4.25 (td, J = 7.73, 7.73, 3.00 Hz, 1 H), 3.85 (m, 1 H), 2.85 (m, 1 H), 2.67 (br d, J = 1.96 Hz, 1 H), 2.60 (d, J = 7.09 Hz, 3 H), 2.50 (m, 2 H), 2.37 (s, 3 H), 2.20 (m, 1 H), 1.86 (br d, J = 13.45 Hz, 1 H), 1.66 (m, 3 H), 1.52 (br s, 1 H), 1.29 (q, J = 12.76, 12.76, 12.76 Hz, 2 H), 1.10 (m, 2 H). LC/MS: m/z [M + H]⁺ 447.3, Rt (I = 220 nm): 2.39 min (LC/MS method A), calc. m/z: 447.22. |
| 288 | 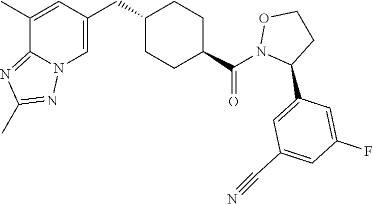 [SMILES: Cc1nc2c(C)cc(C[C@H]3CC[C@H](C(=O)N4OCC[C@H]4c4cc(F)cc(C#N)c4)CC3)cn2n1] | ¹H NMR (400 MHz, DMSO-d₆): δ ppm 8.50 (s, 1 H), 7.76 (d, J = 7.92 Hz, 1 H), 7.58 (s, 1 H), 7.46 (br d, J = 9.41 Hz, 1 H), 7.35 (s, 1 H), 5.35 (dd, J = 8.50, 6.54 Hz, 1 H), 4.25 (td, J = 7.73, 7.73, 2.87 Hz, 1 H), 3.86 (m, 1 H), 2.87 (m, 1 H), 2.67 (m, 1 H), 2.50 (m, 2 H), 2.49 (s, 3 H), 2.47 (s, 3 H), 2.21 (m, 1 H), 1.89 (br d, J = 12.72 Hz, 1 H), 1.70 (br d, J = 12.47 Hz, 3 H), 1.57 (br s, 1 H), 1.30 (m, 2 H), 1.04 (m, 2 H). LC/MS: m/z [M + H]⁺ 462.3, Rt (I = 220 nm): 2.13 min (LC/MS method A), calc. m/z: 462.23. |
| 289 | 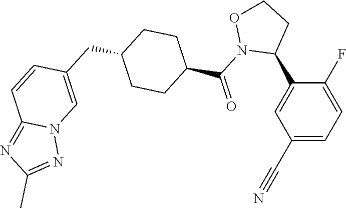 [SMILES: Cc1nc2ccc(C[C@H]3CC[C@H](C(=O)N4OCC[C@H]4c4cc(C#N)ccc4F)CC3)cn2n1] | 1H NMR (400.23 MHz, DMSO-d6) d ppm 8.65 (s, 1 H), 7.87 (ddd, J = 8.50, 4.83, 2.08 Hz, 1 H), 7.67 (d, J = 6.73 Hz, 1 H), 7.63 (d, J = 9.06 Hz, 1 H), 7.49 (m, 2 H), 5.45 (m, 1 H), 4.28 (td, J = 7.76, 7.76, 2.57 Hz, 1 H), 3.88 (m, 1 H), 2.88 (m, 1 H), 2.68 (m, 1 H), 2.56 (d, J = 7.09 Hz, 2 H), 2.45 (s, 2 H), 2.18 (m, 1 H), 1.91 (br d, J = 12.47 Hz, 1 H), 1.71 (br d, J = 8.56 Hz, 3 H), 1.57 (m, 1 H), 1.30 (m, 2 H), 1.05 (m, 2 H). LC/MS: m/z [M + H]⁺ 448.3, Rt (I = 220 nm): 1.97 min (LC/MS method A), calc. m/z: 462.23. |
| 290 | 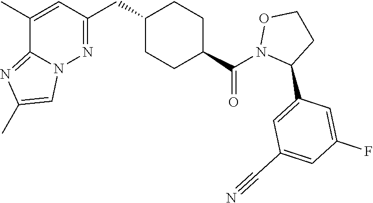 [SMILES: Cc1cn2nc(C[C@H]3CC[C@H](C(=O)N4OCC[C@H]4c4cc(F)cc(C#N)c4)CC3)cc(C)c2n1] | ¹H NMR (400 MHz, DMSO-d₆): δ ppm 8.28 (br s, 1 H), 7.76 (d, J = 8.66 Hz, 1 H), 7.58 (s, 1 H), 7.46 (br d, J = 9.78 Hz, 2 H), 5.35 (m, 1 H), 4.26 (m, 1 H), 3.86 (m, 1 H), 2.88 (m, 1 H), 2.73 (br d, J = 6.60 Hz, 2 H), 2.69 (m, 1 H), 2.58 (s, 3 H), 2.22 (m, 1 H), 1.90 (br d, J = 11.49 Hz, 1 H), 1.72 (br d, J = 11.74 Hz, 4 H), 1.32 (m, 2 H), 1.11 (m, 2 H). LC/MS: m/z [M + H]⁺ 462.2, Rt (I = 220 nm): 1.66 min (LC/MS method A), calc. m/z: 455.23. |
| 291 | 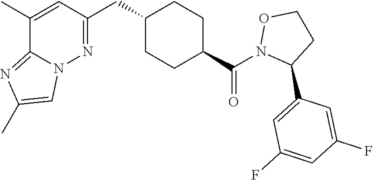 [SMILES: Cc1cn2nc(C[C@H]3CC[C@H](C(=O)N4OCC[C@H]4c4cc(F)cc(F)c4)CC3)cc(C)c2n1] | ¹H NMR (400 MHz, DMSO-d₆): δ ppm 8.28 (br s, 1 H), 7.48 (br s, 1 H), 7.12 (tt, J = 9.31, 9.31, 2.25, 2.25 Hz, 1 H), 6.96 (br d, J = 6.60 Hz, 2 H), 5.31 (m, 1 H), 4.24 (td, J = 7.73, 7.73, 3.12 Hz, 1 H), 3.85 (br dd, J = 8.80, 7.46 Hz, 1 H), 2.86 (m, 1 H), 2.73 (m, 3 H), 2.58 (s, 3 H), 2.18 (m, 1 H), 1.89 (br d, J = 13.20 Hz, 1 H), 1.72 (br d, J = 10.76 Hz, 3 H), 1.32 (m, 2 H), 1.12 (m, 2 H). LC/MS: m/z [M + H]⁺ 455.2, Rt (I = 220 nm): 1.78 min (LC/MS method A), calc. m/z: 455.23. |
| 292 | 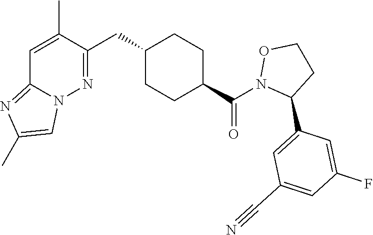 [SMILES: Cc1cn2nc(C[C@H]3CC[C@H](C(=O)N4OCC[C@H]4c4cc(F)cc(C#N)c4)CC3)c(C)cc2n1] | ¹H NMR (400 MHz, DMSO-d₆): δ ppm 8.27 (s, 1 H), 8.13 (s, 1 H), 7.76 (br d, J = 8.44 Hz, 1 H), 7.59 (s, 1 H), 7.46 (br d, J = 9.54 Hz, 1 H), 5.36 (m, 1 H), 4.26 (m, 1 H), 3.87 (m, 1 H), 2.88 (m, 1 H), 2.80 (br d, J = 6.60 Hz, 2 H), 2.73 (br s, 1 H), 2.67 (br s, 3 H), 2.22 (m, 1 H), 1.90 (br d, J = 11.62 Hz, 1 H), 1.78 (br d, J = 12.35 Hz, 3 H), 1.35 (m, 2 H), 1.16 (m, 2 H). LC/MS: m/z [M + H]⁺ 462.3, Rt (I = 220 nm): 1.61 min (LC/MS method A), calc. m/z: 462.23. |
| 293 | 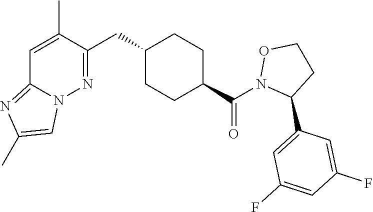 [SMILES: Cc1cn2nc(C[C@H]3CC[C@H](C(=O)N4OCC[C@H]4c4cc(F)cc(F)c4)CC3)c(C)cc2n1] | ¹H NMR (400 MHz, DMSO-d₆): δ ppm 8.28 (s, 1 H), 8.14 (s, 1 H), 7.12 (tt, J = 9.32, 9.32, 2.29, 2.29 Hz, 1 H), 6.96 (br d, J = 6.60 Hz, 2 H), 5.32 (m, 1 H), 4.24 (td, J = 7.79, 7.79, 3.36 Hz, 1 H), 3.85 (m, 1 H), 2.86 (m, 1 H), 2.80 (br d, J = 6.60 Hz, 2 H), 2.69 (m, 1 H), 2.19 (m, 1 H), 1.89 (br d, J = 11.86 Hz, 1 H), 1.80 (br s, 3 H), 1.70 (br d, J = 12.10 Hz, 1 H), 1.36 (m, 2 H), 1.16 (m, 2 H). LC/MS: m/z [M + H]⁺ 455.2, Rt (I = 220 nm): 1.72 min (LC/MS method A), calc. m/z: 455.23. |
| 294 | 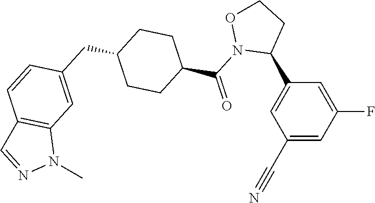 [SMILES: Cn1ncc2ccc(C[C@H]3CC[C@H](C(=O)N4OCC[C@H]4c4cc(F)cc(C#N)c4)CC3)cc21] | ¹H NMR (400 MHz, DMSO-d₆): δ ppm 7.95 (s, 1 H), 7.76 (d, J = 7.96 Hz, 1 H), 7.63 (d, J = 8.19 Hz, 1 H), 7.58 (s, 1 H), 7.46 (br d, J = 9.54 Hz, 1 H), 7.37 (s, 1 H), 6.96 (d, J = 8.31 Hz, 1 H), 5.35 (dd, J = 8.44, 6.60 Hz, 1 H), 4.25 (td, J = 7.76, 7.76, 2.93 Hz, 1 H), 4.00 (s, 3 H), 3.86 (m, 1 H), 2.87 (m, 1 H), 2.67 (br dd, J = 3.67, 1.83 Hz, 1 H), 2.62 (d, J = 6.97 Hz, 2 H), 2.21 (m, 1 H), 1.88 (br d, J = 11.98 Hz, 1 H), 1.71 (br s, 3 H), 1.59 (m, 1 H), 1.30 (m, 2 H), 1.07 (m, 2H). LC/MS: m/z [M + H]⁺ 447.2, Rt (I = 220 nm): 2.51 min (LC/MS method A), calc. m/z: 447.22. |
| 295 | 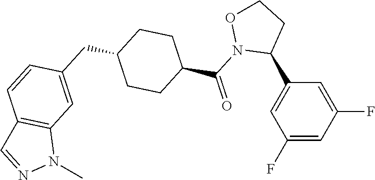 [SMILES: Cn1ncc2ccc(C[C@H]3CC[C@H](C(=O)N4OCC[C@H]4c4cc(F)cc(F)c4)CC3)cc21] | ¹H NMR (400 MHz, DMSO-d₆): δ ppm 7.95 (s, 1 H), 7.63 (d, J = 8.19 Hz, 1 H), 7.37 (s, 1 H), 7.12 (tt, J = 9.31, 9.31, 2.31, 2.31 Hz, 1 H), 6.96 (m, 3 H), 5.31 (dd, J = 8.56, 6.36 Hz, 1 H), 4.23 (td, J = 7.73, 7.73, 3.00 Hz, 1 H), 4.00 (s, 3 H), 3.84 (m, 1 H), 2.85 (m, 1 H), 2.67 (br d, J = 1.71 Hz, 1 H), 2.62 (d, J = 6.97 Hz, 2 H), 2.17 (m, 1 H), 1.88 (br d, J = 12.47 Hz, 1 H), 1.72 (br d, J = 11.0 Hz, 3 H), 1.58 (br dd, J = 11.0, 3.18 Hz, 1 H), 1.30 (m, 2 H), 1.08 (m, 2 H). LC/MS: m/z [M + H]⁺ 440.2, Rt (I = 220 nm): 2.65 min (LC/MS method A), calc. m/z: 440.22. |
| 296 | 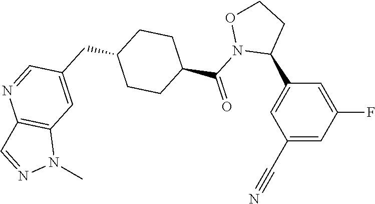 [SMILES: Cn1ncc2ncc(C[C@H]3CC[C@H](C(=O)N4OCC[C@H]4c4cc(F)cc(C#N)c4)CC3)cc21] | ¹H NMR (400 MHz, DMSO-d₆): δ ppm 8.41 (d, J = 1.59 Hz, 1 H), 8.21 (s, 1 H), 7.99 (s, 1 H), 7.76 (d, J = 7.88 Hz, 1 H), 7.59 (s, 1 H), 7.46 (br d, J = 9.66 Hz, 1 H), 5.36 (dd, J = 8.37, 6.54 Hz, 1 H), 4.25 (m, 1 H), 4.06 (s, 3 H), 3.87 (m, 1 H), 2.87 (m, 1 H), 2.69 (m, 3 H), 2.21 (m, 1 H), 1.89 (br d, J = 12.84 Hz, 1 H), 1.71 (br d, J = 9.05 Hz, 3 H), 1.63 (br s, 1 H), 1.31 (m, 2 H), 1.09 (m, 2 H). LC/MS: m/z [M + H]⁺ 448.3, Rt (I = 220 nm): 2.04 min (LC/MS method A), calc. m/z: 448.22. |
| 297 | 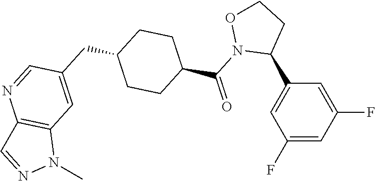 [SMILES: Cn1ncc2ncc(C[C@H]3CC[C@H](C(=O)N4OCC[C@H]4c4cc(F)cc(F)c4)CC3)cc21] | 1H NMR (400.23 MHz, DMSO-d6) d ppm 8.39 (d, J = 1.71 Hz, 1 H), 8.20 (s, 1 H), 7.97 (s, 1 H), 7.12 (tt, J = 9.29, 9.29, 2.32, 2.32 Hz, 1 H), 6.96 (m, 2 H), 5.32 (dd, J = 8.44, 6.48 Hz, 1 H), 4.23 (td, J = 7.76, 7.76, 3.06 Hz, 1 H), 4.06 (s, 3 H), 3.92 (br s, 1 H), 3.84 (m, 4 H), 3.68 (br s, 31 H), 2.85 (m, 1 H), 2.68 (br d, J = 6.97 Hz, 3 H), 2.17 (m, 1 H), 1.89 (br d, J = 12.47 Hz, 1 H), 1.70 (br d, J = 9.54 Hz, 3 H), 1.63 (m, 1 H), 1.31 (m, 2 H), 1.09 (m, 2 H). LC/MS: m/z [M + H]⁺ 441.2, Rt (I = 220 nm): 2.18 min (LC/MS method A), calc. m/z: 441.21. |
| 298 | 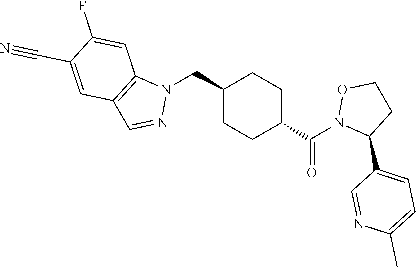 [SMILES: Cc1ccc([C@@H]2CCON2C(=O)[C@H]2CC[C@H](Cn3ncc4cc(C#N)c(F)cc43)CC2)cn1] | ¹H NMR (400 MHz, DMSO-d₆): δ ppm 8.48 (d, J = 6.24 Hz, 1 H), 8.33 (d, J = 2.08 Hz, 1 H), 8.29 (s, 1 H), 7.97 (d, J = 10.39 Hz, 1 H), 7.52 (dd, J = 8.01, 2.38 Hz, 1 H), 7.20 (d, J = 7.95 Hz, 1 H), 5.28 (m, 1 H), 4.29 (d, J = 7.09 Hz, 2 H), 4.24 (m, 1 H), 3.85 (m, 1 H), 2.84 (m, 1 H), 2.67 (br d, J = 1.83 Hz, 1 H), 2.63 (br s, 1 H), 2.43 (s, 3 H), 2.19 (m, 1 H), 1.88 (m, 1 H), 1.82 (br d, J = 13.33 Hz, 1 H), 1.67 (br d, J = 10.39 Hz, 1 H), 1.54 (br t, J = 11.62, 11.62 Hz, 1 H), 1.26-1.01 (m, 4 H). LC/MS: m/z [M + H]⁺ 448.2, Rt (I = 220 nm): 1.56 min (LC/MS method A), calc. m/z: 448.22. |
| 299 | 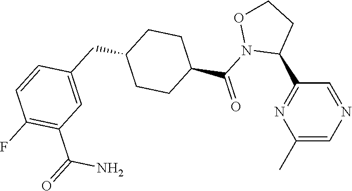 [SMILES: Cc1cncc([C@@H]2CCON2C(=O)[C@H]2CC[C@H](Cc3ccc(F)c(C(N)=O)c3)CC2)n1] | 1H NMR (400.23 MHz, DMSO-d6) d ppm 8.44 (s, 1 H), 8.34 (s, 1 H), 7.62 (br s, 1 H), 7.58 (br s, 1 H), 7.42 (dd, J = 7.09, 2.20 Hz, 1 H), 7.28 (m, 1 H), 7.16 (dd, J = 10.51, 8.44 Hz, 1 H), 5.34 (dd, J = 8.74, 6.30 Hz, 1 H), 4.29 (td, J = 7.61, 7.61, 3.36 Hz, 1 H), 3.91 (m, 1 H), 2.79 (m, 1 H), 2.67 (br dd, J = 3.55, 1.71 Hz, 1 H), 2.43 (m, 1 H), 2.33 (m, 1 H), 2.19 (br dd, J = 14.18, 6.85 Hz, 1 H), 2.11 (m, 1 H), 1.83 (br d, J = 12.23 Hz, 2 H), 1.67 (br d, J = 11.49 Hz, 4 H), 1.48 (br d, J = 3.42 Hz, 2 H), 1.27 (m, 5H). LC/MS: m/z [M + H]⁺ 427.2, Rt (I = 220 nm): 1.82 min (LC/MS method A), calc. m/z: 427.22. |
| 300 | 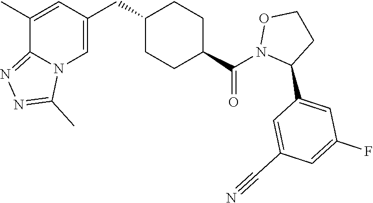 [SMILES: Cc1cc(C[C@H]2CC[C@H](C(=O)N3OCC[C@H]3c3cc(F)cc(C#N)c3)CC2)cn2c(C)nnc12] | 1H NMR (400 MHz, DMSO-d6) δ = 8.08-8.01 (m, 1H), 7.76 (br d, J = 8.8 Hz, 1H), 7.59 (s, 1H), 7.46 (br d, J = 9.3 Hz, 1H), 7.15 (br s, 1H), 5.35 (dd, J = 6.2, 8.1 Hz, 1H), 4.28-4.22 (m, 1H), 3.88-3.83 (m, 1H), 2.92-2.82 (m, 1H), 2.66 (s, 3H), 2.52 (br s, 4H), 2.24- 2.16 (m, 1H), 1.92-1.85 (m, 1H), 1.76-1.67 (m, 3H), 1.63-1.52 (m, 1H), 1.38-1.27 (m, 2H), 1.09-0.97 (m, 2H). LC/MS: m/z [M + H]⁺ 462.4, Rt (I = 220 nm): 0.83 min (LC/MS method B), calc. m/z: 462.23. |
| 301 | 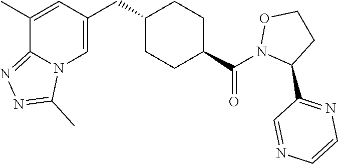 [SMILES: Cc1cc(C[C@H]2CC[C@H](C(=O)N3OCC[C@H]3c3cnccn3)CC2)cn2c(C)nnc12] | 1H NMR (400 MHz, CHLOROFORM-d) δ = 8.66 (d, J = 1.3 Hz, 1H), 8.54-8.52 (m, 1H), 8.50 (d, J = 2.4 Hz, 1H), 7.49 (s, 1H), 6.91 (s, 1H), 5.54 (dd, J = 5.7, 8.9 Hz, 1H), 4.32 (dt, J = 3.8, 7.6 Hz, 1H), 3.98-3.92 (m, 1H), 2.75 (s, 3H), 2.65 (s, 3H), 2.50 (d, J = 7.1 Hz, 2H), 2.00 (br d, J = 12.2 Hz, 1H), 1.91 (br d, J = 1.5 Hz, 3H), 1.83 (br d, J = 12.6 Hz, 4H), 1.56-1.48 (m, 2H), 1.15-1.05 (m, 2H). LC/MS: m/z [M + H]⁺ 421.3, Rt (I = 220 nm): 0.76 min (LC/MS method B), calc. m/z: 421.24. |
| 302 | 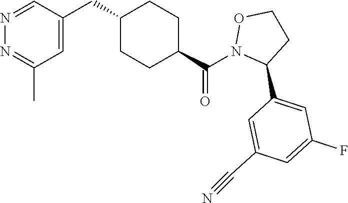 [SMILES: Cc1cc(C[C@H]2CC[C@H](C(=O)N3OCC[C@H]3c3cc(F)cc(C#N)c3)CC2)cnn1] | 1H NMR (400.23 MHz, DMSO-d6) d ppm 8.91 (d, J = 1.96 Hz, 1 H), 7.76 (d, J = 7.96 Hz, 1 H), 7.59 (s, 1 H), 7.46 (br d, J = 9.66 Hz, 1 H), 7.37 (d, J = 1.83 Hz, 1 H), 5.36 (dd, J = 8.56, 6.48 Hz, 1 H), 4.25 (td, J = 7.73, 7.73, 3.00 Hz, 1 H), 3.86 (m, 1 H), 2.87 (dddd, J = 12.13, 9.11, 6.39, 3.06 Hz, 1 H), 2.67 (br t, J = 11.31, 11.31 Hz, 1 H), 2.58 (s, 3 H), 2.21 (m, 1 H), 2.07 (s, 1 H), 1.88 (br d, J = 12.72 Hz, 1 H), 1.65 (m, 3 H), 1.58 (m, 1 H), 1.29 (m, 3 H), 1.05 (m, 2 H). LC/MS: m/z [M + H]⁺ 409.2, Rt (I = 200 nm): 1.78 min (LC/MS method A), calc. m/z: 409.20. |
| 303 | 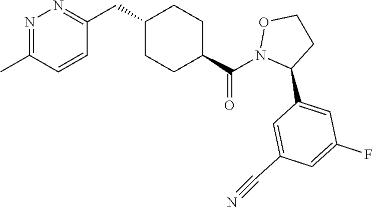 [SMILES: Cc1ccc(C[C@H]2CC[C@H](C(=O)N3OCC[C@H]3c3cc(F)cc(C#N)c3)CC2)nn1] | 1H NMR (400.23 MHz, DMSO-d6) d ppm 7.76 (d, J = 8.65 Hz, 1 H), 7.59 (s, 1 H), 7.46 (m, 1 H), 7.44 (d, J = 3.79 Hz, 2 H), 5.36 (m, 1 H), 4.25 (td, J = 7.76, 7.76, 2.93 Hz, 1 H), 3.86 (ddd, J = 9.26, 8.04, 6.66 Hz, 1 H), 2.87 (dddd, J = 12.13, 9.14, 6.42, 3.00 Hz, 1 H), 2.76 (d, J = 6.85 Hz, 2 H), 2.69 (m, 1 H), 2.57 (s, 3 H), 2.21 (m, 1 H), 1.88 (br d, J = 12.72 Hz, 1 H), 1.68 (m, 4 H), 1.29 (m, 3 H), 1.08 (m, 2 H), 0.79 (m, 1 H). LC/MS: m/z [M + H]⁺ 409.2, Rt (I = 220 nm): 1.72 min (LC/MS method A), calc. m/z: 409.20. |
| 304 | 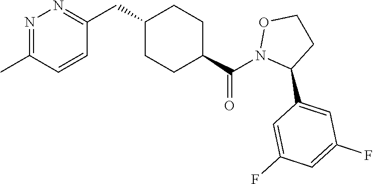 [SMILES: Cc1ccc(C[C@H]2CC[C@H](C(=O)N3OCC[C@H]3c3cc(F)cc(F)c3)CC2)nn1] | 1H NMR (400.23 MHz, DMSO-d6) d ppm 7.44 (m, 2 H), 7.12 (m, 1 H), 6.97 (m, 2 H), 5.32 (m, 1 H), 4.23 (m, 1 H), 3.84 (m, 1 H), 2.86 (m, 1 H), 2.76 (d, J = 6.85 Hz, 1 H), 2.66 (m, 1 H), 2.57 (s, 3 H), 2.17 (m, 1 H), 1.88 (br d, J = 12.35 Hz, 1 H), 1.69 (m, 3 H), 1.29 (m, 3 H), 1.08 (m, 2 H). LC/MS: m/z [M + H]⁺ 402.2, Rt (I = 220 nm): 1.86 min (LC/MS method A), calc. m/z: 402.20. |
| 305 | 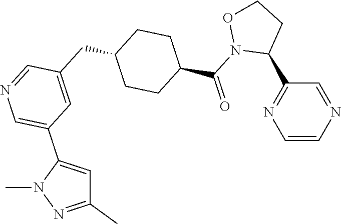 [SMILES: Cc1cc(-c2cncc(C[C@H]3CC[C@H](C(=O)N4OCC[C@H]4c4cnccn4)CC3)c2)n(C)n1] | 1H NMR (400.23 MHz, DMSO-d6) d ppm 8.57 (m, 4 H), 8.42 (d, J = 1.96 Hz, 1 H), 7.74 (t, J = 2.02, 2.02 Hz, 1 H), 6.28 (s, 1 H), 5.40 (dd, J = 8.68, 6.11 Hz, 1 H), 4.30 (td, J = 7.61, 7.61, 3.48 Hz, 1 H), 3.93 (m, 1 H), 3.78 (s, 3 H), 2.81 (m, 1 H), 2.67 (m, 1 H), 2.56 (d, J = 6.97 Hz, 2 H), 2.32 (m, 1 H), 2.17 (s, 3 H), 1.83 (br d, J = 12.35 Hz, 1 H), 1.69 (br d, J = 11.25 Hz, 3 H), 1.57 (td, J = 7.24, 7.24, 3.85 Hz, 1 H), 1.29 (m, 3 H), 1.06 (m, 2 H). LC/MS: m/z [M + H]⁺ 447.2, Rt (I = 220 nm): 1.61 min (LC/MS method A), calc. m/z: 447.25. |
| 306 | 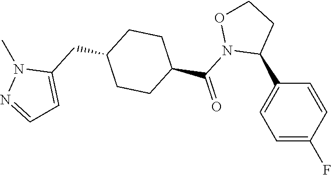 [SMILES: Cn1nccc1C[C@H]1CC[C@H](C(=O)N2OCC[C@H]2c2ccc(F)cc2)CC1] | ¹H NMR (400 MHz, DMSO-d₆): δ ppm 7.29 (m, 2 H), 7.26 (d, J = 1.71 Hz, 1 H), 7.16 (m, 2 H), 6.00 (d, J = 1.71 Hz, 1 H), 5.30 (dd, J = 8.19, 6.60 Hz, 1 H), 4.24 (td, J = 7.73, 7.73, 3.12 Hz, 1 H), 3.83 (m, 1 H), 3.71 (s, 3 H), 2.84 (m, 1 H), 2.67 (br d, J = 1.83 Hz, 1 H), 2.50 (m, 2 H), 2.16 (m, 1 H), 1.89 (br d, J = 16.87 Hz, 1 H), 1.73 (br d, J = 12.96 Hz, 3 H), 1.51 (m, 1 H), 1.33 (m, 2 H), 1.05 (m, 2 H). LC/MS: m/z [M + H]⁺ 372.2, Rt (I = 220 nm): 2.11 min (LC/MS method A), calc. m/z: 372.21. |
| 307 | 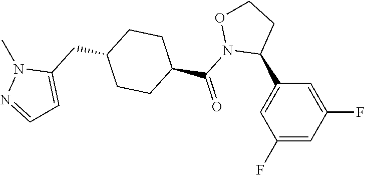 [SMILES: Cn1nccc1C[C@H]1CC[C@H](C(=O)N2OCC[C@H]2c2cc(F)cc(F)c2)CC1] | ¹H NMR (400 MHz, DMSO-d₆): δ ppm 7.27 (d, J = 1.71 Hz, 1 H), 7.12 (m, 1 H), 6.97 (m, 2 H), 6.00 (d, J = 1.71 Hz, 1 H), 5.32 (dd, J = 8.62, 6.30 Hz, 1 H), 4.24 (td, J = 7.73, 7.73, 3.00 Hz, 1 H), 3.84 (m, 1 H), 3.71 (s, 3 H), 2.86 (m, 1 H), 2.67 (m, 1 H), 2.50 (m, 2 H), 2.18 (m, 1 H), 1.89 (br d, J = 15.04 Hz, 1 H), 1.72 (m, 3 H), 1.52 (m, 1 H), 1.33 (m, 2 H), 1.06 (m, 2 H). LC/MS: m/z [M + H]⁺ 390.2, Rt (I = 220 nm): 2.21 min (LC/MS method A), calc. m/z: 390.20. |
| 308 | 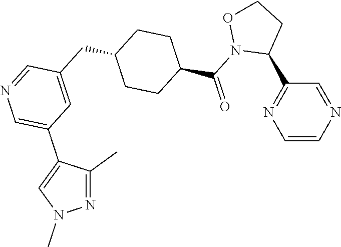 [SMILES: Cc1nn(C)cc1-c1cncc(C[C@H]2CC[C@H](C(=O)N3OCC[C@H]3c3cnccn3)CC2)c1] | 1H NMR (400.23 MHz, DMSO-d6) d ppm 8.58 (m, 3 H), 8.47 (d, J = 2.20 Hz, 1 H), 8.23 (d, J = 1.96 Hz, 1 H), 7.99 (s, 1 H), 7.60 (t, J = 1.96, 1.96 Hz, 1 H), 5.40 (dd, J = 8.80, 6.11 Hz, 1 H), 4.30 (td, J = 7.64, 7.64, 3.55 Hz, 1 H), 3.93 (m, 1 H), 3.79 (s, 3 H), 2.81 (m, 1 H), 2.67 (m, 1 H), 2.30 (s, 3 H), 1.83 (br d, J = 12.10 Hz, 1 H), 1.70 (br d, J = 10.76 Hz, 3 H), 1.55 (m, 1 H), 1.27 (m, 4 H), 1.04 (m, 2 H), 0.85 (m, 1 H). LC/MS: m/z [M + H]⁺ 447.2, Rt (I = 220 nm): 1.25 min (LC/MS method A), calc. m/z: 447.25. |
| 309 | 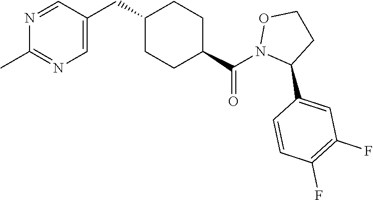 [SMILES: Cc1ncc(C[C@H]2CC[C@H](C(=O)N3OCC[C@H]3c3ccc(F)c(F)c3)CC2)cn1] | ¹H NMR (400 MHz, DMSO-d₆): δ ppm 8.50 (s, 2 H), 7.39 (dt, J = 10.73, 8.51, 8.51 Hz, 1 H), 7.28 (m, 1 H), 7.11 (m, 1 H), 5.29 (dd, J = 8.38, 6.54 Hz, 1 H), 4.23 (td, J = 7.76, 7.76, 3.06 Hz, 1 H), 3.83 (m, 1 H), 2.84 (m, 1 H), 2.67 (m, 1 H), 2.57 (s, 3 H), 2.44 (s, 2 H), 2.17 (m, 1 H), 1.86 (br d, J = 12.72 Hz, 1 H), 1.66 (br d, J = 11.86 Hz, 3 H), 1.50 (m, 1 H), 1.29 (dtd, J = 12.87, 12.70, 12.70, 3.30 Hz, 2 H), 1.02 (m, 2 H). LC/MS: m/z [M + H]⁺ 402.2, Rt (I = 220 nm): 2.09 min (LC/MS method A), calc. m/z: 402.20. |
| 310 | 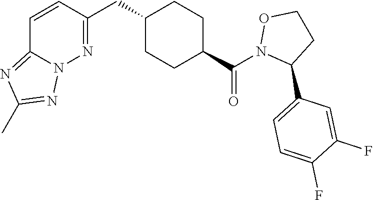 [SMILES: Cc1nc2ccc(C[C@H]3CC[C@H](C(=O)N4OCC[C@H]4c4ccc(F)c(F)c4)CC3)nn2n1] | ¹H NMR (400 MHz, DMSO-d₆): δ ppm 8.20 (d, J = 9.29 Hz, 1 H), 7.53 (d, J = 9.29 Hz, 1 H), 7.39 (dt, J = 10.70, 8.47, 8.47 Hz, 1 H), 7.29 (ddd, J = 11.68, 7.89, 1.96 Hz, 1 H), 7.11 (ddd, J = 6.36, 4.22, 2.14 Hz, 1 H), 5.29 (m, 1 H), 4.24 (td, J = 7.76, 7.76, 2.93 Hz, 1 H), 3.84 (m, 1 H), 2.84 (m, 1 H), 2.77 (d, J = 6.85 Hz, 2 H), 2.67 (m, 1 H), 2.50 (s, 3 H), 2.17 (m, 1 H), 1.87 (br d, J = 12.35 Hz, 1 H), 1.77 (br dd, J = 6.97, 3.67 Hz, 1 H), 1.71 (br d, J = 14.79 Hz, 3 H), 1.32 (m, 2 H), 1.12 (m, 2 H). LC/MS: m/z [M + H]⁺ 442.1, Rt (I = 220 nm): 2.11 min (LC/MS method A), calc. m/z: 442.21. |
| 311 | 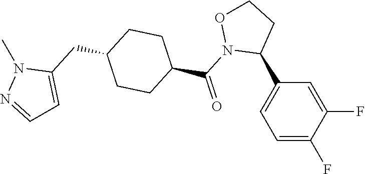 [SMILES: Cn1nccc1C[C@H]1CC[C@H](C(=O)N2OCC[C@H]2c2ccc(F)c(F)c2)CC1] | ¹H NMR (400 MHz, DMSO-d₆): δ ppm 7.41 (m, 1 H), 7.31 (m, 1 H), 7.26 (s, 1 H), 7.11 (m, 1 H), 6.00 (d, J = 1.71 Hz, 1 H), 5.30 (dd, J = 8.38, 6.42 Hz, 1 H), 4.24 (td, J = 7.73, 7.73, 3.00 Hz, 1 H), 3.84 (m, 1 H), 3.71 (s, 3 H), 2.85 (m, 1 H), 2.67 (m, 1 H), 2.50 (m, 2 H), 2.17 (m, 1 H), 1.87 (br d, J = 12.59 Hz, 1 H), 1.72 (m, 3 H), 1.51 (m, 1 H), 1.32 (m, 2 H), 1.06 (m, 2 H). LC/MS: m/z [M + H]⁺ 390.2, Rt (I = 220 nm): 2.19 min (LC/MS method A), calc. m/z: 390.20. |
| 312 | 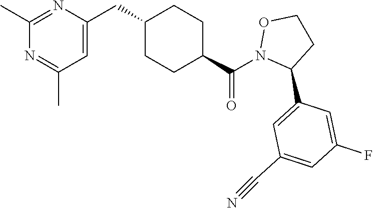 [SMILES: Cc1cc(C[C@H]2CC[C@H](C(=O)N3OCC[C@H]3c3cc(F)cc(C#N)c3)CC2)nc(C)n1] | ¹H NMR (400 MHz, DMSO-d₆) δ ppm: 8.04 (s, 1 H), 7.29 (m, 2 H), 7.21 (d, J = 12.1 Hz, 1 H), 7.15 (m, 2 H), 5.29 (dd, J = 8.3 Hz, 6.5 Hz, 1 H), 4.23 (m, 1 H), 3.83 (m, 1 H), 2.84 (m, 1 H), 2.68 (s, 3 H), 2.66 (m, 1 H), ~2.49 (d, 2 H, below solvent signal), 2.16 (m, 1 H), 1.85 (m, 1 H), 1.74-1.56 (m, 4 H), 1.36-1.24 (m, 2 H), 1.10-0.96 (m, 2 H). LC/MS: m/z [M + H]⁺ 423.3, Rt (I = 220 nm): 1.88 min (LC/MS method A), calc. m/z: 423.5. |
| 313 | 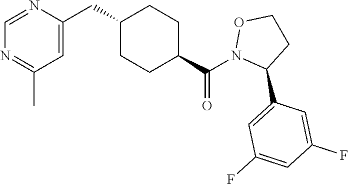 [SMILES: Cc1cc(C[C@H]2CC[C@H](C(=O)N3OCC[C@H]3c3cc(F)cc(F)c3)CC2)ncn1] | ¹H NMR (400 MHz, DMSO-d₆) δ ppm: 8.62-8.56 (m, 3 H), 8.21 (s, 1 H), 6.90 (s, 1 H), 5.39 (dd, J = 8.3 Hz, 6.5 Hz, 1 H), 4.30 (m, 1 H), 3.94 (s, 3 H), 3.93 (m, 1 H), 2.81 (m, 1 H), 2.66 (m, 1 H), ~2.51 (d, J = 6.9 Hz, 2 H, below solvent signal), ~2.48 (m, 1 H, below solvent signal), 2.41 (s, 3 H), 1.83 (m, 1 H), 1.72-1.58 (m, 4 H), 1.36-1.23 (m, 2 H), 1.09-0.97 (m, 2 H). LCMS: Method A, m/z 402.3 [M + 1]+, RT 2.11 min (LC/MS method A) min (LC/MS method A), calc. m/z: 402.5. |
| 314 | 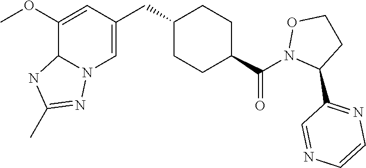 [SMILES: COC1=CC(C[C@H]2CC[C@H](C(=O)N3OCC[C@H]3c3cnccn3)CC2)=CN2N=C(C)NC12] | ¹H NMR (400 MHz, DMSO-d₆): δ ppm 8.64-8.54 (m, 3H), 8.21 (s, 1H), 6.91 (s, 1H), 5.40 (dd, J = 6.4, 8.6 Hz, 1H), 4.30 (dt, J = 3.3, 7.6 Hz, 1H), 3.97-3.89 (m, 4H), 2.86-2.76 (m, 1H), 2.67 (br d, J = 1.8 Hz, 1H), 2.53 (br s, 2H), 2.41 (s, 3H), 1.86-1.79 (m, 1H), 1.69 (br d, J = 11.7 Hz, 3H), 1.61 (br dd, J = 6.8, 10.6 Hz, 1H), 1.39 (s, 1H), 1.37-1.22 (m, 3H), 1.04 (br d, J = 11.7 Hz, 2H). LC/MS: m/z [M + H]⁺ 437.0, Rt (I = 220 nm): 0.63 min (LC/MS method B), calc. m/z: 437.5. |
| 315 | 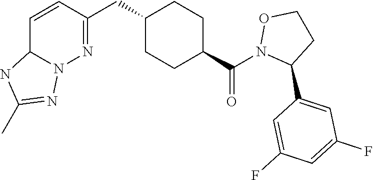 [SMILES: CC1=NN2N=C(C[C@H]3CC[C@H](C(=O)N4OCC[C@H]4c4cc(F)cc(F)c4)CC3)C=CC2N1] | ¹H NMR (400 MHz, DMSO-d₆) δ ppm: 8.30 (d, J = 2.0 Hz, 1 H), 7.94 (s, 1 H), 7.71 (bd, 1 H), 7.29 (m, 2 H), 7.15 (m, 2 H), 5.29 (dd, J = 8.3 Hz, 6.5 Hz, 1 H), 4.23 (m, 1 H), 3.83 (m, 1 H), 2.84 (m, 1 H), 2.65 (m, 1 H), 2.55 (d, J = 6.9 Hz, 2 H), 2.35 (s, 3 H), 2.15 (m, 1 H), 1.85 (m, 1 H), 1.71-1.55 (m, 4 H), 1.36-1.24 (m, 2 H), 1.11-0.98 (m, 2 H). LC/MS: m/z [M + H]⁺ 442.3, Rt (I = 220 nm): 0.91 min (LC/MS method B), calc. m/z: 442.5. |
| 316 | 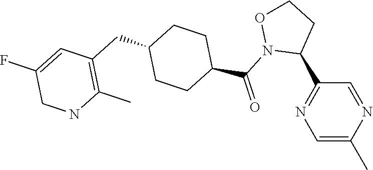 [SMILES: CC1=C(C[C@H]2CC[C@H](C(=O)N3OCC[C@H]3c3cnc(C)cn3)CC2)C=C(F)CN1] | ¹H NMR (400 MHz, DMSO-d₆) δ ppm: 8.48 (d, J = 1.2 Hz, 1 H), 8.42 (d, J = 1.2 Hz, 1 H), 8.26 (d, J = 2.8 Hz, 1 H), 7.46 (dd, J = 9.7 Hz, 2.8 Hz, 1 H), 5.35 (dd, J = 8.7 Hz, 6.2 Hz, 1 H), 4.29 (m, 1 H), 3.92 (m, 1 H), 2.78 (m, 1 H), 2.67 (m, 1 H), ~2.50 (d, 2 H, below solvent signal), ~2.48 (m, 1 H, below solvent signal), 2.47 (s, 3 H), 2.43 (s, 3 H), 1.81 (m, 1 H), 1.72-1.62 (m, 3 H), 1.54 (m, 1 H), 1.35-1.24 (m, 2 H), 1.14-1.02 (m, 2 H). LC/MS: m/z [M + H]⁺ 399.2, Rt (I = 220 nm): 1.73 min (LC/MS method A), calc. m/z: 399.5. |
| 317 | 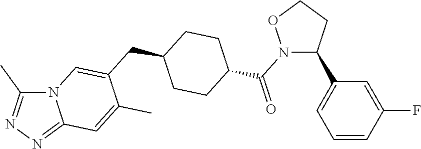 [SMILES: Cc1cc2nnc(C)n2cc1C[C@H]1CC[C@H](C(=O)N2OCC[C@H]2c2cccc(F)c2)CC1] | ¹H NMR (400 MHz, DMSO-d₆) δ ppm: 8.03 (s, 1 H), 7.47 (s, 1 H), 7.39 (m, 1 H), 7.29 (m, 1 H), 7.11 (m, 1 H), 5.30 (dd, J = 8.4 Hz, 6.5 Hz, 1 H), 4.24 (m, 1 H), 3.84 (m, 1 H), 2.85 (m, 1 H), 2.69 (m, 1 H), 2.63 (s, 3 H), ~2.49 (d, 2 H, below solvent signal), 2.34 (s, 3 H), 2.17 (m, 1 H), 1.87 (m, 1 H), 1.77-1.68 (m, 3 H), 1.53 (m, 1 H), 1.39-1.27 (m, 2 H), 1.16-1.02 (m, 2 H). LC/MS: m/z [M + H]⁺ 437.2, Rt (I = 220 nm): 1.74 min (LC/MS method A), calc. m/z: 437.5. |
| 318 | 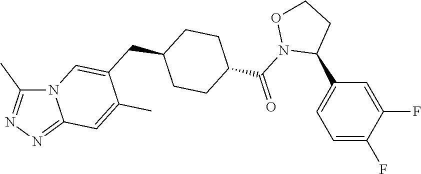 [SMILES: Cc1cc2nnc(C)n2cc1C[C@H]1CC[C@H](C(=O)N2OCC[C@H]2c2ccc(F)c(F)c2)CC1] | ¹H NMR (400 MHz, DMSO-d₆) δ ppm: 8.03 (s, 1 H), 7.47 (s, 1 H), 7.39 (m, 1 H), 7.29 (m, 1 H), 7.11 (m, 1 H), 5.30 (dd, J = 8.4 Hz, 6.5 Hz, 1 H), 4.24 (m, 1 H), 3.84 (m, 1 H), 2.85 (m, 1 H), 2.69 (m, 1 H), 2.63 (s, 3 H), ~2.49 (d, 2 H, below solvent signal), 2.34 (s, 3 H), 2.17 (m, 1 H), 1.87 (m, 1 H), 1.77-1.68 (m, 3 H), 1.53 (m, 1 H), 1.39-1.27 (m, 2 H), 1.16-1.02 (m, 2 H). LC/MS: m/z [M + H]⁺ 454.2, Rt (I = 220 nm): 1.80 min (LC/MS method A), calc. m/z: 454.5. |
| 319 | 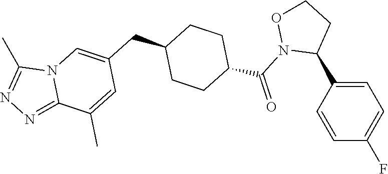 [SMILES: Cc1cc(C[C@H]2CC[C@H](C(=O)N3OCC[C@H]3c3ccc(F)cc3)CC2)cn2c(C)nnc12] | ¹H NMR (400 MHz, DMSO-d₆) δ ppm: 8.92 (s, 1 H), 7.26 (s, 1 H), 7.12 (m, 1 H), 6.96 (m, 2 H), 5.32 (dd, J = 8.5 Hz, 6.5 Hz, 1 H), 4.23 (m, 1 H), 3.84 (m, 1 H), 2.86 (m, 1 H), 2.67 (m, 1 H), 2.57 (d, J = 7.0 Hz, 2 H), 2.42 (s, 3 H), 2.17 (m, 1 H), 1.87 (m, 1 H), 1.78-1.65 (m, 4 H), 1.37-1.24 (m, 2 H), 1.14-0.99 (m, 2 H). LC/MS: m/z [M + H]⁺ 437.2, Rt (I = 220 nm): 1.80 min (LC/MS method A), calc. m/z: 437.5. |
| 320 | 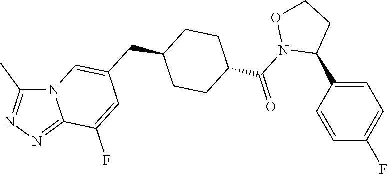 [SMILES: Cc1nnc2c(F)cc(C[C@H]3CC[C@H](C(=O)N4OCC[C@H]4c4ccc(F)cc4)CC3)cn12] | ¹H NMR (400 MHz, DMSO-d₆) δ ppm: 7.76 (m, 1 H), 7.59 (s, 1 H), 7.46 (m, 1 H), 7.12 (s, 1 H), 5.36 (dd, J = 8.5 Hz, 6.5 Hz, 1 H), 4.25 (m, 1 H), 3.86 (m, 1 H), 2.87 (m, 1 H), 2.67 (m, 1 H), 2.55 (s, 3 H), 2.54 (d, 2 H, signal overlap), 2.40 (s, 3 H), 2.21 (m, 1 H), 1.88 (m, 1 H), 1.74-1.65 (m, 4 H), 1.36-1.25 (m, 2 H), 1.13-1.00 (m, 2 H). LC/MS: m/z [M + H]⁺ 441.1, Rt (I = 220 nm): 2.00 min (LC/MS method A), calc. m/z: 441.5. |
| 321 | 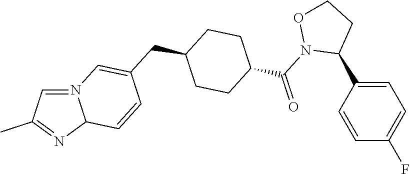 | ¹H NMR (400 MHz, DMSO-d₆) δ ppm: 8.20 (d, J = 9.3 Hz, 1 H), 7.53 (d, J = 9.3 Hz, 1 H), 7.12 (m, 1 H), 6.96 (m, 2 H), 5.32 (dd, J = 8.3 Hz, 6.5 Hz, 1 H), 4.23 (m, 1 H), 3.84 (m, 1 H), 2.85 (m, 1 H), 2.77 (d, J = 6.9 Hz, 2 H), 2.69 (m, 1 H), ~2.50 (s, 3 H, below solvent), 2.17 (m, 1 H), 1.89 (m, 1 H), 1.80-1.66 (m, 4 H), 1.38- 1.32 (m, 2 H), 1.18-1.04 (m, 2 H). LC/MS: m/z [M + H]⁺ 423.2, Rt (I = 220 nm): 2.02 min (LC/MS method A), calc. m/z: 423.5. |
| 322 | 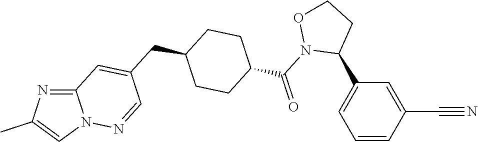 [SMILES: Cc1cn2ncc(C[C@H]3CC[C@H](C(=O)N4OCC[C@H]4c4cccc(C#N)c4)CC3)cc2n1] | ¹H NMR (400 MHz, DMSO-d₆) δ ppm: 8.31 (d, J = 2.0 Hz, 1 H), 7.94 (s, 1 H), 7.75-7.69 (m, 3 H), 7.61- 7.54 (m, 2 H), 5.34 (dd, J = 8.5 Hz, 6.5 Hz, 1 H), 4.25 (m, 1 H), 3.85 (m, 1 H), 2.88 (m, 1 H), 2.68 (m, 1 H), 2.56 (d, J = 6.9 Hz, 2 H), 2.35 (s, 3 H), 2.19 (m, 1 H), 1.88 (m, 1 H), 1.72-1.56 (m, 4 H), 1.37-1.24 (m, 2 H), 1.12-0.98 (m, 2 H). LC/MS: m/z [M + H]⁺ 430.5, Rt (I = 220 nm): 1.50 min (LC/MS method A), calc. m/z: 430.5. |
| 323 | 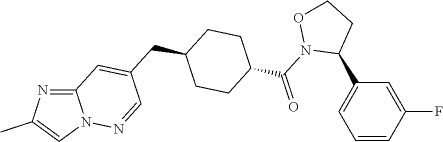 [SMILES: Cc1cn2ncc(C[C@H]3CC[C@H](C(=O)N4OCC[C@H]4c4cccc(F)c4)CC3)cc2n1] | ¹H NMR (400 MHz, DMSO-d₆) δ ppm: 8.03 (s, 1 H), 7.47 (s, 1 H), 7.38 (m, 1 H), 7.12-7.04 (m, 3 H), 5.31 (dd, J = 8.3 Hz, 6.5 Hz, 1 H), 4.24 (m, 1 H), 3.84 (m, 1 H), 2.86 (m, 1 H), 2.69 (m, 1 H), 2.64 (s, 3 H), ~2.49 (d, 2 H, below solvent signal), 2.34 (s, 3 H), 2.17 (m, 1 H), 1.87 (m, 1 H), 1.77-1.68 (m, 3 H), 1.54 (m, 1 H), 1.39-1.27 (m, 2 H), 1.16-1.02 (m, 2 H). LC/MS: m/z [M + H]⁺ 423.3, Rt (I = 220 nm): 1.62 min (LC/MS method A), calc. m/z: 423.5. |
| 324 | 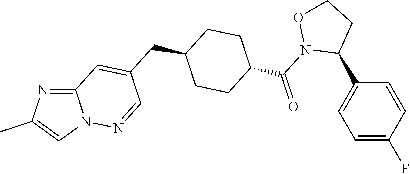 [SMILES: Cc1cn2ncc(C[C@H]3CC[C@H](C(=O)N4OCC[C@H]4c4ccc(F)cc4)CC3)cc2n1] | ¹H NMR (400 MHz, DMSO-d₆) δ ppm : 8.31 (d, J = 2.0 Hz, 1 H), 7.94 (s, 1 H), 7.75-7.69 (m, 3 H), 7.61- 7.54 (m, 2 H), 5.34 (dd, J = 8.5 Hz, 6.5 Hz, 1 H), 4.25 (m, 1 H), 3.85 (m, 1 H), 2.88 (m, 1 H), 2.68 (m, 1 H), 2.56 (d, J = 6.9 Hz, 2 H), 2.35 (s, 3 H), 2.19 (m, 1 H), 1.88 (m, 1 H), 1.72-1.56 (m, 4 H), 1.37-1.24 (m, 2 H), 1.12-0.98 (m, 2 H). LC/MS: m/z [M + H]⁺ 423.3, Rt (I = 220 nm): 1.61 min (LC/MS method A), calc. m/z: 423.5. |
| 325 | 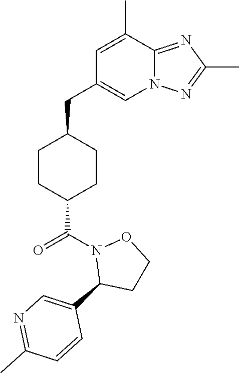 | ¹H NMR (400 MHz, Chloroform-d) δ ppm = 8.45 (d, J = 2.2 Hz, 1 H), 8.10 (s, 1 H), 7.52 (dd, J = 8.0 Hz, 2.2 Hz, 1 H), 7.13 (d, J = 8.0 Hz, 1 H), 7.08 (s, 1 H), 5.38 (dd, J = 8.6 Hz, 6.1 Hz, 1 H), 4.27 (m, 1 H), 3.89 (m, 1 H), 2.84 (m, 1 H), 2.73 (m, 1 H), 2.60 (s, 6 H), 2.54 (s, 3 H), 2.51 (d, J = 7.1 Hz, 2 H), 2.33 (m, 1 H), 1.96 (m, 1 H), 1.82-1.75 (m, 3 H), 1.58 (m, 1 H), 1.48 (m, 2 H), 1.07 (m, 2 H). LC/MS: m/z [M + H]⁺ 434.3, Rt (I = 220 nm): 0.444 min (LC/MS method Q), calc. m/z: 434.3. |
| 326 | 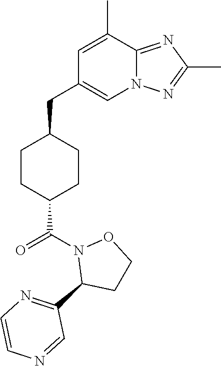 [SMILES: Cc1nc2c(C)cc(C[C@H]3CC[C@H](C(=O)N4OCC[C@H]4c4cnccn4)CC3)cn2n1] | ¹H NMR (400 MHz, Chloroform-d) δ ppm = 8.64 (s, 1H), 8.50 (br d, J = 12.2 Hz, 2H), 8.09 (br s, 1H), 7.07 (br s, 1H), 5.62-5.44 (m, 1H), 4.42-4.26 (m, 1H), 4.03-3.83 (m, 1H), 2.90-2.66 (m, 3H), 2.59 (s, 6H), 2.52-2.44 (m, 2H), 2.02-1.91 (m, 1H), 1.89-1.71 (m, 3H), 1.65-1.38 (m, 3H), 1.17-0.98 (m, 2H). LC/MS: m/z [M + H]⁺ 421.33, Rt (I = 220 nm): 0.473 min (LC/MS method Q), calc. m/z: 421.2. |
| 327 | 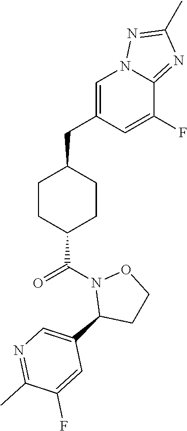 [SMILES: Cc1nc2c(F)cc(C[C@H]3CC[C@H](C(=O)N4OCC[C@H]4c4cnc(C)c(F)c4)CC3)cn2n1] | ¹H NMR (400 MHz, DMSO-d6) δ = 8.64 (s, 1H), 8.53- 8.48 (m, 2H), 8.09 (br s, 1H), 7.07 (br s, 1H), 5.57- 5.46 (m, 1H), 4.36-4.25 (m, 1H), 4.00-3.88 (m, 1H), 2.87-2.65 (m, 3H), 2.58 (s, 6H), 2.50 (br d, J = 6.8 Hz, 2H), 2.01-1.93 (m, 1H), 1.79 (br s, 3H), 1.65- 1.40 (m, 3H), 1.16-0.97 (m, 2H). LC/MS: m/z [M + H]⁺ 456.3, Rt (I = 220 nm): 0.568 min (LC/MS method Q), calc. m/z: 455.2. |
| 328 | 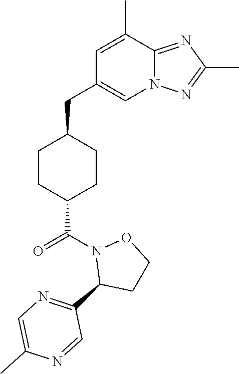 | ¹H NMR (400 MHz, Chloroform-d) δ ppm = 8.52 (d, J = 1.3 Hz, 1H), 8.39 (d, J = 0.9 Hz, 1H), 8.13-8.07 (m, 1H), 7.12-7.06 (m, 1H), 5.54-5.43 (m, 1H), 4.37- 4.25 (m, 1H), 3.99-3.89 (m, 1H), 2.85-2.63 (m, 3H), 2.60 (s, 6H), 2.55 (s, 3H), 2.51 (d, J = 7.1 Hz, 2H), 2.02-1.88 (m, 1H), 1.85-1.76 (m, 3H), 1.64- 1.53 (m, 1H, below water signal), 1.52-1.39 (m, 2H), 1.17-0.99 (m, 2H). LC/MS: m/z [M + H]⁺ 435.3, Rt (I = 220 nm): 0.454 min (LC/MS method Q), calc. m/z: 435.2. |
| 329 | 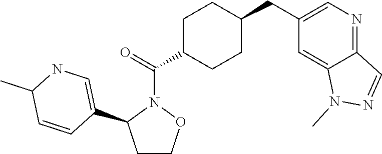 | ¹H NMR (400 MHz, Chloroform-d) δ ppm = 8.48- 8.34 (m, 2H), 8.17 (s, 1H), 7.54-7.44 (m, 2H), 7.11 (d, J = 7.9 Hz, 1H), 5.46-5.29 (m, 1H), 4.34-4.18 (m, 1H), 4.08 (s, 3H), 3.95-3.85 (m, 1H), 2.88-2.78 (m, 1H), 2.78-2.66 (m, 3H), 2.53 (s, 3H), 2.41-2.27 (m, 1H), 2.01-1.91 (m, 1H), 1.89-1.73 (m, 4H), 1.57- 1.41 (m, 2H), 1.23-1.02 (m, 2H). LC/MS: m/z [M + H]⁺ 402.2, Rt (I = 220 nm): 0.889 min (LC/MS method S), calc. m/z: 420.2. |
| 330 | 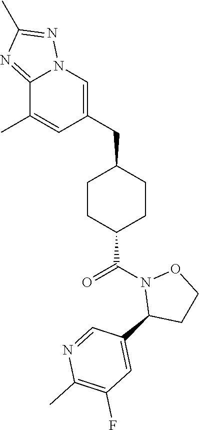 [SMILES: Cc1nc2c(C)cc(C[C@H]3CC[C@H](C(=O)N4OCC[C@H]4c4cnc(C)c(F)c4)CC3)cn2n1] | ¹H NMR (400 MHz, DMSO-d6) δ = 8.45 (s, 1H), 8.23 (s, 1H), 7.46 (dd, J = 10.5, 1.5 Hz, 1H), 7.28 (s, 1H), 5.40-5.29 (m, 1H), 4.33-4.23 (m, 1H), 3.96-3.82 (m, 1H), 2.94-2.81 (m, 1H), 2.73-2.61 (m, 1H), 2.50- 2.48 (m, 2H, below solvent signal), 2.47 (s, 3H), 2.44 (s, 3H), 2.41 (d, J = 2.8 Hz, 3H), 2.31-2.19 (m, 1H), 1.89-1.81 (m, 1H), 1.75-1.63 (m, 3H), 1.61- 1.48 (m, 1H), 1.38-1.21 (m, 2H), 1.12-0.91 (m, 2H). LC/MS: m/z [M + H]⁺ 452.3, Rt (I = 220 nm): 0.49 min (LC/MS method Q), calc. m/z: 452.2. |
| 331 | 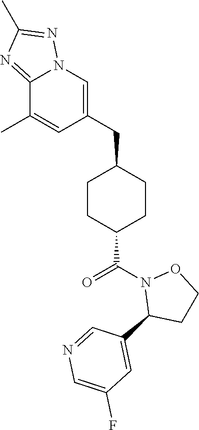 [SMILES: Cc1nc2c(C)cc(C[C@H]3CC[C@H](C(=O)N4OCC[C@H]4c4cncc(F)c4)CC3)cn2n1] | ¹H NMR (400 MHz, Chloroform-d) δ ppm = 8.39 (br t, J = 1.4 Hz, 1H), 8.37 (d, J = 2.7 Hz, 1H), 8.10 (s, 1H), 7.33 (td, J = 2.2, 9.1 Hz, 1H), 7.08 (s, 1H), 5.50-5.42 (m, 1H), 4.34-4.21 (m, 1H), 3.97-3.81 (m, 1H), 2.95- 2.83 (m, 1H), 2.82-2.69 (m, 1H), 2.60 (s, 6H), 2.55- 2.48 (m, 2H), 2.39-2.29 (m, 1H), 2.03-1.95 (m, 1H), 1.86-1.74 (m, 3H), 1.65-1.41 (m, 2H, below water signal), 1.21-0.99 (m, 2H). LC/MS: m/z [M + H]⁺ 438.3, Rt (I = 220 nm): 0.491 min (LC/MS method Q), calc. m/z: 438.2. |
| 332 | 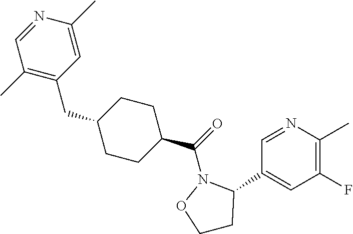 [SMILES: Cc1cc(C[C@H]2CC[C@H](C(=O)N3OCC[C@H]3c3cnc(C)c(F)c3)CC2)c(C)cn1] | ¹H NMR (400 MHz, DMSO-d6) δ = 8.23 (s, 1H), 8.16 (s, 1H), 7.50-7.43 (m, 1H), 6.96 (s, 1H), 5.38-5.27 (m, 1H), 4.32-4.21 (m, 1H), 3.92-3.79 (m, 1H), 2.94- 2.77 (m, 1H), 2.73-2.60 (m, 1H), 2.46-2.39 (m, 5H), 2.37 (s, 3H), 2.29-2.20 (m, 1H), 2.18 (s, 3H), 1.89-1.78 (m, 1H), 1.73-1.60 (m, 3H), 1.56-1.45 (m, 1H), 1.35-1.20 (m, 2H), 1.16-1.00 (m, 2H). LC/MS: m/z [M + H]⁺ 412.2, Rt (I = 220 nm): 0.995 min (LC/MS method S), calc. m/z: 412.2. |
| 333 | 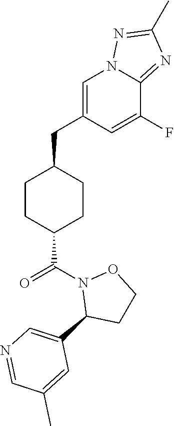 [SMILES: Cc1cncc([C@@H]2CCON2C(=O)[C@H]2CC[C@H](Cc3cc(F)c4nc(C)nn4c3)CC2)c1] | ¹H NMR (400 MHz, DMSO-d6) δ = 8.58 (s, 1H), 8.29 (dd, J = 7.4, 1.7 Hz, 2H), 7.52-7.47 (m, 1H), 7.47- 7.43 (m, 1H), 5.37-5.24 (m, 1H), 4.32-4.21 (m, 1H), 3.93-3.78 (m, 1H), 2.93-2.81 (m, 1H), 2.72-2.60 (m, 1H), 2.55 (d, J = 7.0 Hz, 2H), 2.47 (s, 3H), 2.28 (s, 3H), 2.25-2.15 (m, 1H), 1.89-1.82 (m, 1H), 1.74- 1.63 (m, 3H), 1.59 (br s, 1H), 1.37-1.24 (m, 2H), 1.12-0.96 (m, 2H). LC/MS: m/z [M + H]⁺ 438.3, Rt (I = 220 nm): 0.877 min (LC/MS method R), calc. m/z: 438.2. |
| 334 | 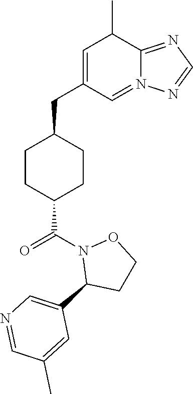 | ¹H NMR (400 MHz, DMSO-d6) δ = 8.58 (s, 1H), 8.37 (s, 1H), 8.29 (dd, J = 7.3, 1.5, Hz, 2H), 7.47-7.43 (m, 1H), 7.35 (s, 1H), 5.35-5.24 (m, 1H), 4.34-4.21 (m, 1H), 3.90-3.79 (m, 1H), 2.92-2.80 (m, 1H), 2.72- 2.61 (m, 1H), 2.54-2.51 (m, 5H, below solvent signal), 2.28 (s, 3H), 2.26-2.14 (m, 1H), 1.89-1.80 (m, 1H), 1.76-1.64 (m, 3H), 1.64-1.52 (m, 1H), 1.38- 1.21 (m, 2H), 1.13-0.96 (m, 2H). LC/MS: m/z [M + H]⁺ 420.2, Rt (I = 220 nm): 0.408 min (LC/MS method Q), calc. m/z: 420.2. |
| 335 | 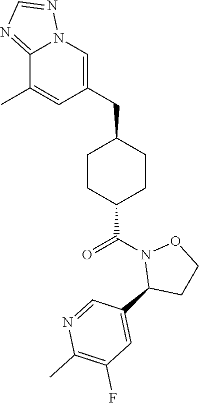 [SMILES: Cc1ncc([C@@H]2CCON2C(=O)[C@H]2CC[C@H](Cc3cc(C)c4ncnn4c3)CC2)cc1F] | ¹H NMR (400 MHz, DMSO-d6) δ = 8.58 (s, 1H), 8.37 (s, 1H), 8.22 (s, 1H), 7.52-7.39 (m, 1H), 7.35 (s, 1H), 5.37-5.32 (m, 1H), 4.32-4.23 (m, 1H), 3.92-3.78 (m, 1H), 2.91-2.75 (m, 1H), 2.73-2.60 (m, 1H), 2.55- 2.51 (m, 5H, below solvent signal), 2.43-2.38 (m, 3H), 2.30-2.19 (m, 1H), 1.90-1.78 (m, 1H), 1.76- 1.63 (m, 3H), 1.63-1.50 (m, 1H), 1.38-1.22 (m, 2H), 1.13-0.95 (m, 2H). LC/MS: m/z [M + H]⁺ 438.4 Rt (I = 220 nm): 0.475 min (LC/MS method Q), calc. m/z: 438.2. |
| 336 | 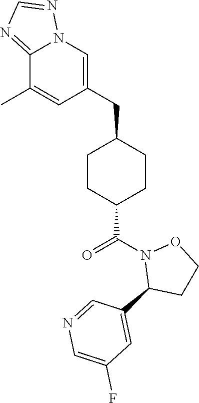 [SMILES: Cc1cc(C[C@H]2CC[C@H](C(=O)N3OCC[C@H]3c3cncc(F)c3)CC2)cn2ncnc12] | ¹H NMR (400 MHz, DMSO-d6) δ = 8.59 (s, 1H), 8.48 (d, J = 2.8 Hz, 1H), 8.37 (s, 2H), 7.57 (dt, J = 9.8, 2.1 Hz, 1H), 7.37-7.33 (m, 1H), 5.42-5.33 (m, 1H), 4.33- 4.21 (m, 1H), 3.95-3.79 (m, 1H), 2.95-2.85 (m, 1H), 2.74-2.62 (m, 1H), 2.56-2.51 (m, 5H), 2.35- 2.18 (m, 1H), 1.93-1.83 (m, 1H), 1.76-1.64 (m, 3H), 1.64-1.53 (m, 1H), 1.37-1.22 (m, 2H), 1.14-0.95 (m, 2H). LC/MS: m/z 424.30 [M + H]⁺, Rt (I = 220 nm): 0.477 min (LC/MS method Q), calc. m/z:. 424.21. |
| 337 | 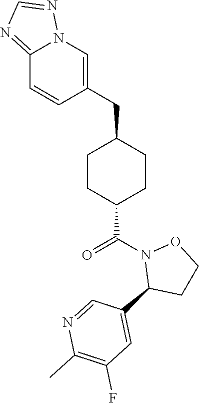 [SMILES: Cc1ncc([C@@H]2CCON2C(=O)[C@H]2CC[C@H](Cc3ccc4ncnn4c3)CC2)cc1F] | ¹H NMR (400 MHz, DMSO-d6) δ = 8.77 (s, 1H), 8.42 (s, 1H), 8.22 (s, 1H), 7.76 (d, J = 9.0 Hz, 1H), 7.57- 7.51 (m, 1H), 7.46 (br d, J = 9.5 Hz, 1H), 5.39-5.30 (m, 1H), 4.32-4.21 (m, 1H), 3.94-3.78 (m, 1H), 2.91-2.79 (m, 1H), 2.74-2.61 (m, 1H), 2.58 (br d, J = 7.0 Hz, 2H), 2.41 (d, J = 2.8 Hz, 3H), 2.30-2.17 (m, 1H), 1.90-1.80 (m, 1H), 1.74-1.63 (m, 3H), 1.63-1.52 (m, 1H), 1.40-1.20 (m, 2H), 1.14-0.95 (m, 2H). LC/MS: m/z [M + H]⁺ 424.3, Rt xx (I = 220 nm): 0.451 min (LC/MS method Q), calc. m/z: 424.2. |
| 338 | 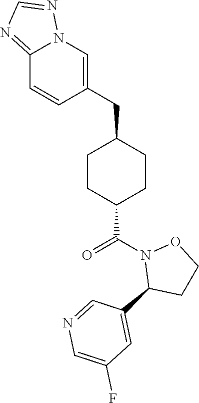 [SMILES: O=C([C@H]1CC[C@H](Cc2ccc3ncnn3c2)CC1)N1OCC[C@H]1c1cncc(F)c1] | ¹H NMR (400 MHz, Chloroform-d) δ ppm = 8.42- 8.36 (m, 3H), 8.32 (s, 1H), 7.72 (d, J = 9.3 Hz, 1H), 7.39 (dd, J = 9.0, 1.7 Hz, 1H), 7.34 (td, J = 9.0, 2.3 Hz, 1H), 5.45 (dd, J = 8.8, 6.4 Hz, 1H), 4.29 (td, J = 7.8, 2.9 Hz, 1H), 3.93-3.85 (m, 1H), 2.94-2.84 (m, 1H), 2.82-2.71 (m, 1H), 2.67-2.55 (m, 2H), 2.41- 2.27 (m, 1H), 2.06-1.97 (m, 1H), 1.89-1.74 (m, 3H), 1.71-1.57 (m, 1H), 1.57-1.42 (m, 2H), 1.22-1.02 (m, 2H). LC/MS: m/z 410.3 [M + H]⁺, Rt (I = 220 nm): 0.534 min (LC/MS method Q), calc. m/z: 410.2. |
| 339 | 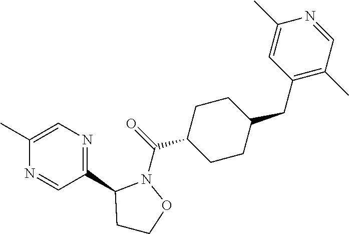 | ¹H NMR (400 MHz, MeOD-d4) δ = 8.47 (s, 1H), 8.44 (s, 1H), 8.12 (s, 1H), 7.04 (s, 1H), 5.41 (dd, J = 5.9, 8.5 Hz, 1H), 4.41-4.31 (m, 1H), 4.09-3.99 (m, 1H), 2.98-2.70 (m, 2H), 2.66-2.55 (m, 1H), 2.55-2.51 (m, 5H), 2.44 (s, 3H), 2.26 (s, 3H), 1.95-1.88 (m, 1H), 1.87-1.67 (m, 3H), 1.58 (ttd, J = 3.9, 7.5, 14.8 Hz, 1H), 1.46-1.31 (m, 2H), 1.22-1.07 (m, 2H). LC/MS: m/z 395.3 [M + H]⁺, Rt (I = 220 nm): 0.415 min (LC/MS method Q), calc. m/z:. |
| 340 | 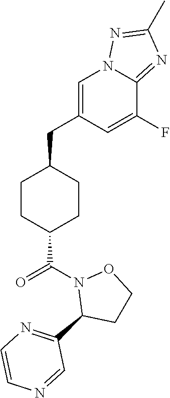 [SMILES: Cc1nc2c(F)cc(C[C@H]3CC[C@H](C(=O)N4OCC[C@H]4c4cnccn4)CC3)cn2n1] | ¹H NMR (400 MHz, Chloroform-d) δ ppm = 8.65 (d, J = 1.4 Hz, 1H), 8.53-8.50 (m, 1H), 8.49 (d, J = 2.5 Hz, 1H), 8.14-8.05 (m, 1H), 7.04 (dd J = 1.2 10.2 Hz, 1H), 5.52 (dd, J = 8.9, 5.8 Hz, 1H), 4.39-4.25 (m, 1H), 4.01-3.86 (m, 1H), 2.88-2.65 (m, 3H), 2.61 (s, 3H), 2.55 (d, J = 7.1 Hz, 2H), 2.06-1.92 (m, 1H), 1.89-1.77 (m, 4H), 1.49-1.39 (m, 2H, below water signal), 1.20-1.01 (m, 2H). LC/MS: m/z 425.3 [M + H]⁺, Rt (I = 220 nm): 0.549 min (LC/MS method Q), calc. m/z: 425.2. |
| 341 | 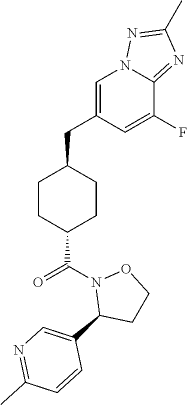 [SMILES: Cc1ccc([C@@H]2CCON2C(=O)[C@H]2CC[C@H](Cc3cc(F)c4nc(C)nn4c3)CC2)cn1] | ¹H NMR (400 MHz, Chloroform-d) δ ppm = 8.44 (d, J = 2.3 Hz, 1H), 8.11 (s, 1H), 7.50 (dd, J = 8.0, 2.4 Hz, 1H), 7.11 (d, J = 8.0 Hz, 1H), 7.04 (dd, J = 10.1, 1.3 Hz, 1H), 5.43-5.32 (m, 1H), 4.31-4.23 (m, 1H), 3.96- 3.82 (m, 1H), 2.89-2.78 (m, 1H), 2.78-2.68 (m, 1H), 2.61 (s, 3H), 2.58-2.54 (m, 2H), 2.53 (s, 3H), 2.39-2.28 (m, 1H), 2.03-1.95 (m, 1H), 1.87-1.74 (m, 3H), 1.74-1.64 (m, 1H, below water signal), 1.54- 1.41 (m, 2H), 1.16-1.00 (m, 2H). LC/MS: m/z 438.3 [M + H]⁺, Rt (I = 220 nm): 0.496 min (LC/MS method Q), calc. m/z: 438.2. |
| 342 | 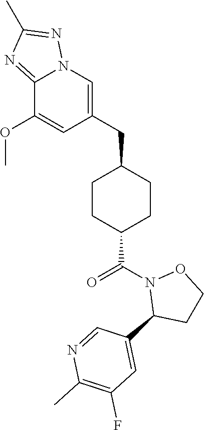 [SMILES: COc1cc(C[C@H]2CC[C@H](C(=O)N3OCC[C@H]3c3cnc(C)c(F)c3)CC2)cn2nc(C)nc12] | ¹H NMR (400 MHz, Chloroform-d) δ ppm = 8.27 (s, 1H), 7.89 (s, 1H), 7.26-7.24 (m, 1H, below solvent signal), 6.57 (s, 1H), 5.40 (dd, J = 8.6, 6.3 Hz, 1H), 4.27 (td, J = 7.8, 3.2 Hz, 1H), 4.02 (s, 3H), 3.94-3.83 (m, 1H), 2.91-2.81 (m, 1H), 2.79-2.68 (m, 1H), 2.58 (s, 3H), 2.55-2.51 (m, 2H), 2.50 (d, J = 2.8 Hz, 3H), 2.39-2.27 (m, 1H), 2.00-1.92 (m, 1H), 1.87-1.72 (m, 3H), 1.66-1.55 (m, 1H), 1.55-1.39 (m, 2H), 1.19- 0.97 (m, 2H). LC/MS: m/z 468.3 [M + H]⁺, Rt (I = 220 nm): 0.544 min (LC/MS method Q), calc. m/z: 468.2. |
| 343 | 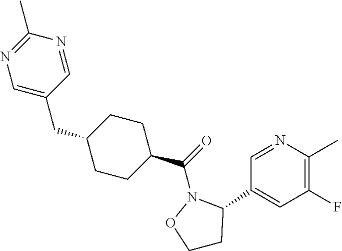 [SMILES: Cc1ncc(C[C@H]2CC[C@H](C(=O)N3OCC[C@H]3c3cnc(C)c(F)c3)CC2)cn1] | ¹H NMR (400 MHz, MeOD-d4) δ = 8.52 (s, 2H), 8.24- 8.17 (m, 1H), 7.46 (dd, J = 10.1, 1.6 Hz, 1H), 5.44- 5.34 (m, 1H), 4.37-4.28 (m, 1H), 4.02-3.91 (m, 1H), 2.99-2.88 (m, 1H), 2.86-2.73 (m, 1H), 2.65 (s, 3H), 2.54 (d, J = 7.1 Hz, 2H), 2.47 (d, J = 2.9 Hz, 3H), 2.41- 2.28 (m, 1H), 2.01-1.90 (m, 1H), 1.83-1.71 (m, 3H), 1.64-1.51 (m, 1H), 1.51-1.32 (m, 2H), 1.20- 1.00 (m, 2H). LC/MS: m/z 399.3 [M + H]⁺, Rt (I = 220 nm): 0.528 min (LC/MS method Q), calc. m/z: 399.2. |
| 344 | 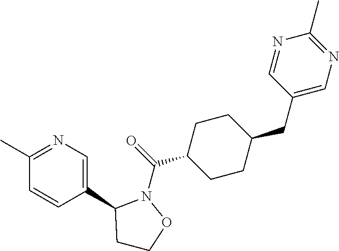 [SMILES: Cc1ccc([C@@H]2CCON2C(=O)[C@H]2CC[C@H](Cc3cnc(C)nc3)CC2)cn1] | ¹H NMR (400 MHz, MeOD-d4) δ = 8.52 (s, 2H), 8.34 (d, J = 2.0 Hz, 1H), 7.65 (dd, J = 8.1, 2.2 Hz, 1H), 7.28 (d, J = 7.8 Hz, 1H), 5.40-5.30 (m, 1H), 4.36- 4.27 (m, 1H), 4.02-3.90 (m, 1H), 3.00-2.89 (m, 1H), 2.87-2.73 (m, 1H), 2.65 (s, 3H), 2.54 (d, J = 6.8 Hz, 2H), 2.50 (s, 3H), 2.38-2.24 (m, 1H), 1.98-1.89 (m, 1H), 1.85-1.70 (m, 3H), 1.63-1.50 (m, 1H), 1.49- 1.32 (m, 2H), 1.19-0.97 (m, 2H). LC/MS: m/z 381.2 [M + H]⁺, Rt (I = 220 nm): 0.394 min (LC/MS method Q), calc. m/z: 381.2. |
| 345 | 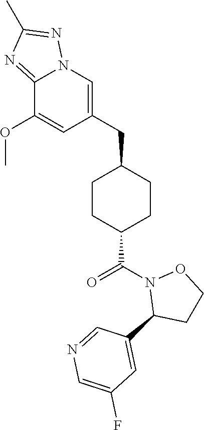 [SMILES: COc1cc(C[C@H]2CC[C@H](C(=O)N3OCC[C@H]3c3cncc(F)c3)CC2)cn2nc(C)nc12] | ¹H NMR (400 MHz, MeOD-d4) δ ppm = 8.43-8.33 (m, 2H), 8.06 (s, 1H), 7.56 (td, J = 9.2, 2.0 Hz, 1H), 6.93 (s, 1H), 5.46-5.38 (m, 1H), 4.39-4.30 (m, 1H), 4.02 (s, 3H), 4.01-3.93 (m, 1H), 3.04-2.92 (m, 1H), 2.89-2.76 (m, 1H), 2.59 (d, J = 7.0 Hz, 2H), 2.50 (s, 3H), 2.40-2.29 (m, 1H), 2.05-1.92 (m, 1H), 1.86- 1.74 (m, 3H), 1.74-1.61 (m, 1H), 1.51-1.37 (m, 2H), 1.23-1.04 (m, 2H). LC/MS: m/z 454.2 [M + H]⁺, Rt (I = 220 nm): 0.447 min (LC/MS method Q), calc. m/z: 454.2. |
| 346 | 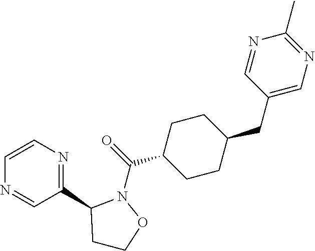 [SMILES: Cc1ncc(C[C@H]2CC[C@H](C(=O)N3OCC[C@H]3c3cnccn3)CC2)cn1] | ¹H NMR (400 MHz, Chloroform-d) δ ppm = 8.67- 8.62 (m, 1H), 8.54-8.51 (m, 1H), 8.49 (d, J = 2.4 Hz, 1H), 8.43 (s, 2H), 5.53 (dd, J = 8.9, 5.8 Hz, 1H), 4.32 (td, J = 7.7, 3.9 Hz, 1H), 3.94 (q, J = 8.1 Hz, 1H), 2.86- 2.66 (m, 6H), 2.47 (d, J = 7.0 Hz, 2H), 2.02-1.94 (m, 1H), 1.85-1.76 (m, 3H), 1.60-1.42 (m, 3H), 1.14- 1.00 (m, 2H). LC/MS: m/z 368.1 [M + H]⁺, Rt (I = 220 nm): 0.414 min (LC/MS method Q), calc. m/z: 368.2. |
| 347 | 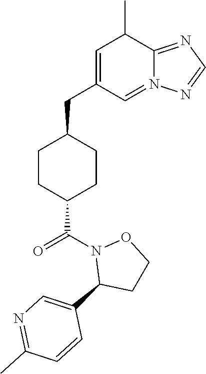 | ¹H NMR (400 MHz, DMSO-d6) δ ppm = 8.60-8.56 (m, 1H), 8.37 (s, 1H), 8.34 (d, J = 2.1 Hz, 1H), 7.52 (dd, J = 8.0, 2.4 Hz, 1H), 7.39-7.30 (m, 1H), 7.21 (d, J = 8.1 Hz, 1H), 5.34-5.25 (m, 1H), 4.30-4.19 (m, 1H), 3.92-3.80 (m, 1H), 2.90-2.79 (m, 1H), 2.72- 2.59 (m, 1H), 2.54-2.51 (m, 5H, below solvent signal), 2.43 (s, 3H), 2.27-2.11 (m, 1H), 1.90-1.77 (m, 1H), 1.75-1.65 (m, 3H), 1.64-1.53 (m, 1H), 1.37- 1.21 (m, 2H), 1.12-0.96 (m, 2H). LC/MS: m/z 420.3 [M + H]⁺, Rt (I = 220 nm): 0.484 min (LC/MS method Q), calc. m/z: 420.2. |
| 348 | 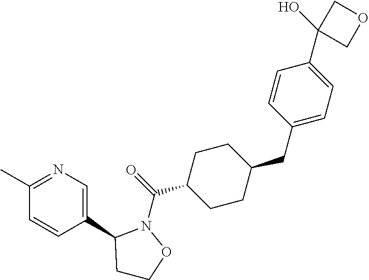 [SMILES: Cc1ccc([C@@H]2CCON2C(=O)[C@H]2CC[C@H](Cc3ccc(C4(O)COC4)cc3)CC2)cn1] | ¹H NMR (400 MHz, MeOD-d4) δ ppm = 8.36-8.27 (m, 1H), 7.68-7.62 (m, 1H), 7.51 (d, J = 8.1 Hz, 2H), 7.28 (d, J = 8.1 Hz, 1H), 7.20 (d, J = 8.0 Hz, 2H), 5.40- 5.29 (m, 1H), 4.29-4.80 (m, 2H, signal under solvent), 4.63-4.53 (m, 2H), 4.39-4.25 (m, 1H), 4.00-3.90 (m, 1H), 2.99-2.87 (m, 1H), 2.87-2.71 (m, 1H), 2.55-2.47 (m, 5H), 2.39-2.24 (m, 1H), 1.98- 1.85 (m, 1H), 1.83-1.71 (m, 3H), 1.59-1.48 (m, 1H), 1.48-1.28 (m, 2H), 1.16-0.97 (m, 2H). LC/MS: m/z 437.3 [M + H]⁺, Rt (I = 220 nm): 0.751 min (LC/MS method C), calc. m/z: 437.2. |
| 349 | 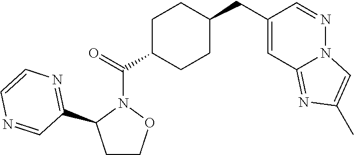 | ¹H NMR (400 MHz, Chloroform-d) δ ppm = 8.65 (d, J = 1.1 Hz, 1 H), 8.52 (dd, J = 2.5 Hz, 1.1 Hz, 1 H), 8.49 (d, J = 2.5 Hz, 1 H), 8.12 (d, J = 1.9 Hz, 1 H), 7.69 (bs, 1 H), 7.67 (bs, 1 H), 5.52 (dd, J = 8.8 Hz, 5.8 Hz, 1 H), 4.32 (m, 1 H), 3.94 (m, 1 H), 2.86-2.64 (m, 3 H), 2.59 (d, J = 7.1, 2 H), 2.50 (s, 3 H), 1.98 (m, 1 H), 1.82 (m, 3 H), 1.63 (m, 1 H), 1.49 (m, 2 H), 1.11 (m, 2 H). LC/MS: m/z 07.3 [M + H]⁺, Rt (I = 220 nm): 0.718 min (LC/MS method Q), calc. m/z: 0.7.2. |
| 350 | 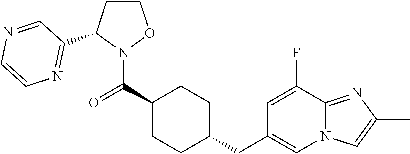 [SMILES: Cc1cn2cc(C[C@H]3CC[C@H](C(=O)N4OCC[C@H]4c4cnccn4)CC3)cc(F)c2n1] | ¹H NMR (600 MHz, DMSO-d6) δ = 8.60 (dd, J = 2.5, 1.6 Hz, 1H), 8.57 (d, J = 1.1 Hz, 1H), 8.56 (d, J = 2.6 Hz, 1H), 8.11 (s, 1H), 7.71 (d, J = 2.6 Hz, 1H), 6.97 (dd, J = 12.1, 0.9 Hz, 1H), 5.40 (dd, J = 8.7, 6.3 Hz, 1H), 4.30 (td, J = 7.7, 3.6 Hz, 1H), 3.98-3.87 (m, 1H), 2.85-2.77 (m, 1H), 2.71-2.60 (m, 1H), 2.49- 2.45 (m, 1H), 2.45-2.42 (m, 2H), 2.32 (s, 3H), 1.85- 1.80 (m, 1H), 1.77-1.66 (m, 3H), 1.59-1.49 (m, 1H), 1.35-1.25 (m, 2H), 1.08-0.92 (m, 2H). LC/MS: m/z 424.2 [M + H]⁺, Rt (I = 220 nm): 1.200 min (LC/MS method A), calc. m/z: 424.2. |
| 351 | 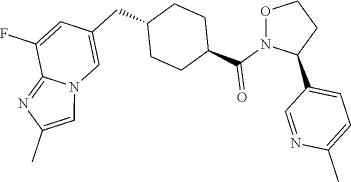 [SMILES: Cc1ccc([C@@H]2CCON2C(=O)[C@H]2CC[C@H](Cc3cc(F)c4nc(C)cn4c3)CC2)cn1] | 1H NMR (400 MHz, CHLOROFORM-d) δ = 8.44 (d, J = 2.2 Hz, 1H), 7.62 (s, 1H), 7.50 (dd, J = 2.3, 8.0 Hz, 1H), 7.33 (d, J = 2.7 Hz, 1H), 7.11 (d, J = 8.1 Hz, 1H), 6.68 (d, J = 11.0 Hz, 1H), 5.38 (dd, J = 6.2, 8.6 Hz, 1H), 4.26 (dt, J = 3.4, 7.8 Hz, 1H), 3.96-3.81 (m, 1H), 2.90- 2.79 (m, 2H), 2.77-2.65 (m, 1H), 2.53 (s, 3H), 2.48-2.48 (m, 1H), 2.47-2.44 (m, 5H), 2.37-2.30 (m, 1H), 1.97 (br d, J = 6.7 Hz, 1H), 1.77-1.72 (m, 2H), 1.57-1.53 (m, 1H), 1.51-1.43 (m, 2H), 1.30-1.23 (m, 1H), 1.10-1.00 (m, 2H). LC/MS: m/z 437.2 [M + H]⁺, Rt (I = 220 nm): 0.478 min (LC/MS method Q), calc. m/z: 437.2. |
| 352 | 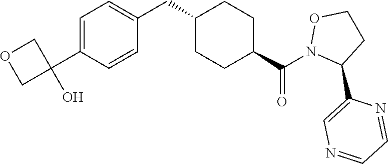 [SMILES: O=C([C@H]1CC[C@H](Cc2ccc(C3(O)COC3)cc2)CC1)N1OCC[C@H]1c1cnccn1] | ¹H NMR (400 MHz, MeOD-d4) δ ppm = 8.61-8.58 (m, 1H), 8.59-8.56 (m, 1H), 8.51-8.49 (m, 1H), 7.51 (d, J = 8.0 Hz, 2H), 7.20 (d, J = 8.0 Hz, 2H), 5.45 (br dd, J = 9.0, 6.0 Hz, 1H), 4.58 (s, 4H), 4.39-4.33 (m, 1H), 4.07-4.01 (m, 1H), 2.92-2.86 (m, 1H), 2.82- 2.76 (m, 1H), 2.62-2.57 (m, 1H), 2.55-2.50 (m, 2H), 1.94-1.88 (m, 1H), 1.82-1.74 (m, 3H), 1.59-1.51 (m, 2H), 1.44-1.34 (m, 2H), 1.12-1.03 (m, 2H). LC/MS: m/z 424.3 [M + H]⁺, Rt (I = 220 nm): 0.819 min (LC/MS method C), calc. m/z: 424.2. |
| 353 | 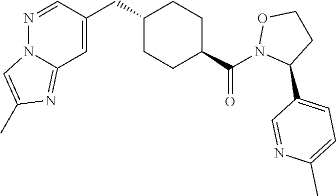 [SMILES: Cc1ccc([C@@H]2CCON2C(=O)[C@H]2CC[C@H](Cc3cnn4cc(C)nc4c3)CC2)cn1] | 1H NMR (400 MHz, CHLOROFORM-d) δ = 8.42 (d, J = 2.1 Hz, 1H), 8.05 (d, J = 2.0 Hz, 1H), 7.66 (s, 1H), 7.54 (s, 1H), 7.48 (dd, J = 2.3, 8.0 Hz, 1H), 7.09 (d, J = 8.1 Hz, 1H), 5.35 (dd, J = 6.2, 8.6 Hz, 1H), 4.24 (dt, J = 3.3, 7.8 Hz, 1H), 3.91-3.82 (m, 1H), 2.88-2.76 (m, 1H), 2.75- 2.64 (m, 1H), 2.54 (br d, J = 7.0 Hz, 2H), 2.50 (s, 3H), 2.47-2.43 (m, 3H), 2.35-2.26 (m, 1H), 1.94 (br d, J = 12.5 Hz, 1H), 1.82-1.71 (m, 3H), 1.64-1.53 (m, 1H), 1.49-1.40 (m, 2H), 1.13-1.01 (m, 2H). LC/MS: m/z 420.3 [M + H]⁺, Rt (I = 220 nm): 0.528 min (LC/MS method C), calc. m/z: 420.2. |
| 354 | 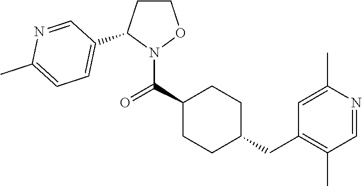 [SMILES: Cc1ccc([C@@H]2CCON2C(=O)[C@H]2CC[C@H](Cc3cc(C)ncc3C)CC2)cn1] | ¹H NMR (400 MHz, MeOD-d4) δ ppm = 8.34 (d, J = 1.7 Hz, 1H), 8.12 (s, 1H), 7.65 (dd, J = 8.1, 2.2 Hz, 1H), 7.28 (d, J = 8.1 Hz, 1H), 7.04 (s, 1H), 5.42-5.31 (m, 1H), 4.32 (td, J = 7.8, 3.1 Hz, 1H), 4.07-3.88 (m, 1H), 3.00-2.89 (m, 1H), 2.88-2.74 (m, 1H), 2.53 (br d, J = 7.1 Hz, 2H), 2.50 (s, 3H), 2.44 (s, 3H), 2.37- 2.24 (m, 4H), 1.99-1.89 (m, 1H), 1.82-1.68 (m, 3H), 1.63-1.53 (m, 1H), 1.46-1.31 (m, 2H), 1.26-1.05 (m, 2H). LC/MS: m/z 394.2 [M + H]⁺, Rt (I = 220 nm): 0.367 min (LC/MS method Q), calc. m/z: 394.2. |
| 355 | 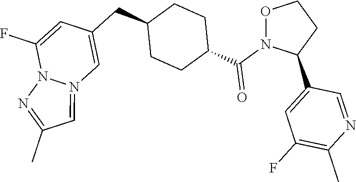 | ¹H NMR (400 MHz, MeOD-d4) δ ppm = 8.34-8.28 (m, 1H), 8.26-8.21 (m, 1H), 7.48 (dd, J = 10.1, 1.3 Hz, 1H), 7.32 (s, 1H), 5.46-5.36 (m, 1H), 4.33 (td, J = 7.8, 2.9 Hz, 1H), 4.04-3.90 (m, 1H), 3.02-2.90 (m, 1H), 2.82-2.70 (m, 1H), 2.60 (s, 2H), 2.58-2.54 (m, 6H), 2.49 (d, J = 2.8 Hz, 3H), 2.41-2.29 (m, 1H), 1.86-1.75 (m, 1H), 1.72-1.57 (m, 3H), 1.56-1.36 (m, 4H), 0.97 (s, 3H). LC/MS: m/z 438.3 [M + H]⁺, Rt (I = 220 nm): 0.523 min (LC/MS method Q), calc. m/z: 438.2. |
| 356 | 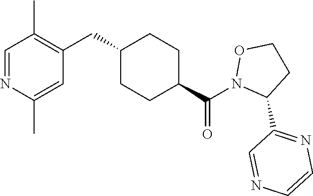 [SMILES: Cc1cc(C[C@H]2CC[C@H](C(=O)N3OCC[C@@H]3c3cnccn3)CC2)c(C)cn1] | 1H NMR (400.23 MHz, DMSO-d6) d ppm 8.58 (m, 3 H), 8.16 (s, 1 H), 6.96 (s, 1 H), 5.40 (dd, J = 8.80, 6.11 Hz, 1 H), 4.30 (td, J = 7.61, 7.61, 3.48 Hz, 1 H), 3.94 (m, 1 H), 2.81 (m, 1H), 2.68 (m, 1 H), 2.50 (u), 2.43 (d, J = 7.09 Hz, 2 H), 2.37 (s, 3 H), 2.18 (s, 3 H), 1.82 (br d, J = 12.10 Hz, 1 H), 1.72 (br s, 1 H), 1.67 (br d, J = 13.33 Hz, 2 H), 1.50 (br dd, J = 7.46, 3.91 Hz, 1 H), 1.27 (m, 3 H), 1.08 (m, 2 H). LC/MS: m/z 381.2 [M + H]⁺, Rt (I = 220 nm): 1.150 min (LC/MS method A), calc. m/z: 381.2. |
| 357 | 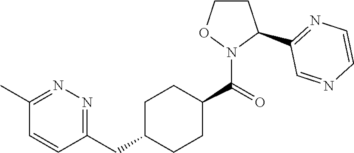 [SMILES: Cc1ccc(C[C@H]2CC[C@H](C(=O)N3OCC[C@H]3c3cnccn3)CC2)nn1] | ¹H NMR (400 MHz, MeOD-d4) δ ppm = 8.59 (s, 2H), 8.50 (d, J = 2.9 Hz, 1H), 7.56-7.49 (m, 2H), 5.45 (br d, J = 2.9 Hz, 1H), 4.36 (td, J = 7.6, 3.9 Hz, 1H), 4.09- 3.99 (m, 1H), 2.94-2.76 (m, 4H), 2.68-2.53 (m, 4H), 1.99-1.89 (m, 1H), 1.86-1.70 (m, 4H), 1.51- 1.32 (m, 2H), 1.25-1.07 (m, 2H). LC/MS: m/z 368.1 [M + H]⁺, Rt (I = 220 nm): 0.386 min (LC/MS method Q), calc. m/z: 368.2. |
| 358 | 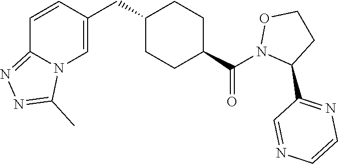 | 1H NMR (400 MHz, CHLOROFORM-d) δ = 8.65 (d, J = 1.3 Hz, 1H), 8.54-8.52 (m, 1H), 8.49 (d, J = 2.5 Hz, 1H), 7.51 (d, J = 9.3 Hz, 1H), 7.01 (d, J = 9.4 Hz, 1H), 5.53 (dd, J = 5.8, 8.8 Hz, 1H), 4.33 (dt, J = 3.9, 7.8 Hz, 1H), 4.02-3.90 (m, 1H), 3.06 (s, 3H), 2.87-2.67 (m, 6H), 2.58-2.48 (m, 2H), 2.02-1.96 (m, 1H), 1.82 (br d, J = 9.8 Hz, 3H), 1.57-1.41 (m, 3H), 1.21-1.06 (m, 2H). LC/MS: m/z 421.3 [M + H]⁺, Rt (I = 220 nm): 0.523 min (LC/MS method F), calc, m/z: 421.2. |
| 359 | 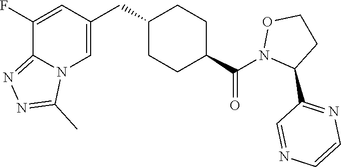 | 1H NMR (400 MHz, CHLOROFORM-d) δ = 8.66 (d, J = 0.8 Hz, 1H), 8.54-8.52 (m, 1H), 8.50 (d, J = 2.5 Hz, 1H), 7.48 (s, 1H), 6.86 (d, J = 10.5 Hz, 1H), 5.53 (dd, J = 5.8, 8.9 Hz, 1H), 4.33 (dt, J = 3.9, 7.8 Hz, 1H), 3.96 (q, J = 8.1 Hz, 1H), 2.87-2.67 (m, 6H), 2.54 (d, J = 7.0 Hz, 2H), 2.03-2.01 (m, 1H), 1.83 (br dd, J = 3.5, 9.4 Hz, 3H), 1.68-1.58 (m, 1H), 1.56-1.42 (m, 2H), 1.17-1.03 (m, 2H). LC/MS: m/z 404.3 [M + H]⁺, Rt (I = 220 nm): 0.592 min (LC/MS method F), calc. m/z: 404.2. |
| 360 | 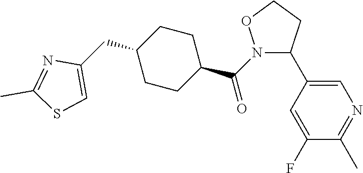 | 1H NMR (400 MHz, CHLOROFORM-d) δ = 8.66 (d, J = 0.8 Hz, 1H), 8.54-8.52 (m, 1H), 8.50 d, J = 2.5 Hz, 1H), 7.48 (s, 1H), 6.86 (d, J = 10.5 Hz, 1H), 5.53 (dd, J = 5.8, 8.9 Hz, 1H), 4.33 (dt, J = 3.9, 7.8 Hz, 1H), 3.96 (q, J = 8.1 Hz, 1H), 2.87-2.67 (m, 6H), 2.54 (d, J = 7.0 Hz, 2H), 2.03-2.01 (m, 1H), 1.83 (br dd, J = 3.5, 9.4 Hz, 3H), 1.68-1.58 (m, 1H), 1.56-1.42 (m, 2H), 1.17-1.03 (m, 2H). LC/MS: m/z 404.3 [M + H]⁺, Rt (I = 220 nm): 0.536 min (LC/MS method Q), calc. m/z: 404.3. |
| 361 | 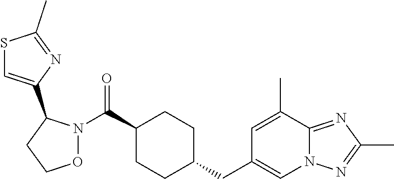 [SMILES: Cc1nc2c(C)cc(C[C@H]3CC[C@H](C(=O)N4OCC[C@H]4c4csc(C)n4)CC3)cn2n1] | ¹H NMR (600 MHz, DMSO-d6) δ = 8.44 (s, 1H), 7.28 (s, 1H), 7.18 (br s, 1H), 5.40-5.30 (m, 1H), 4.25- 4.19 (m, 1H), 3.84 (td, J = 8.2, 7.1 Hz, 1H), 2.73- 2.66 (m, 1H), 2.66-2.59 (m, 4H), 2.50-2.48 (m, 2H, signal below solvent), 2.47 (s, 3H), 2.43 (s, 3H), 2.42- 2.35 (m, 1H), 1.84-1.79 (m, 1H), 1.71-1.65 (m, 3H), 1.60-1.52 (m, 1H), 1.35-1.23 (m, 2H), 1.09- 0.96 (m, 2H). LC/MS: m/z 368.1 [M + H]⁺, Rt (I = 220 nm): 1.850 min (LC/MS method A), calc. m/z: 368.2. |
| 362 | 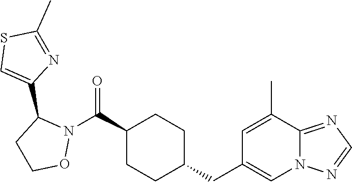 [SMILES: Cc1nc([C@@H]2CCON2C(=O)[C@H]2CC[C@H](Cc3cc(C)c4ncnn4c3)CC2)cs1] | ¹H NMR (600 MHz, DMSO-d6) δ = 8.58 (s, 1H), 8.37 (s, 1H), 7.35 (s, 1H), 7.18 (br s, 1H), 5.42-5.24 (m, 1H), 4.22 (td, J = 7.7, 3.9 Hz, 1H), 3.84 (q, J = 8.0 Hz, 1H), 2.74-2.66 (m, 1H), 2.66-2.60 (m, 4H), 2.54- 2.51 (m, 5H), 2.44-2.33 (m, 1H), 1.87-1.76 (m, 1H), 1.73-1.64 (m, 3H), 1.62-1.52 (m, 1H), 1.40-1.23 (m, 2H), 1.09-0.96 (m, 2H). LC/MS: m/z 426.2 [M + H]⁺, Rt (I = 220 nm): 1.870 min (LC/MS method A), calc. m/z: 426.2. |
| 363 | 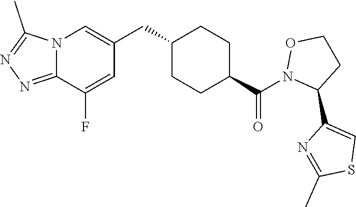 [SMILES: Cc1nc([C@@H]2CCON2C(=O)[C@H]2CC[C@H](Cc3cc(F)c4nnc(C)n4c3)CC2)cs1] | ¹H NMR (600 MHz, DMSO-d6) δ = 8.04 (s, 1H), 7.24- 7.16 (m, 2H), 5.40-5.31 (m, 1H), 4.23 (td, J = 7.3, 4.5 Hz, 1H), 3.84 (q, J = 7.8 Hz, 1H), 2.74-2.67 (m, 4H), 2.66-2.60 (m, 4H), 2.50-2.48 (m, 2H), 2.44- 2.35 (m, 1H), 1.85-1.80 (m, 1H), 1.74-1.65 (m, 3H), 1.65-1.57 (m, 1H), 1.37-1.26 (m, 2H), 1.09-0.98 (m, 2H). LC/MS: m/z 426.2 [M + H]⁺, Rt (I = 220 nm): 1.700 min (LC/MS method A), calc. m/z: 426.2. |
| 364 | 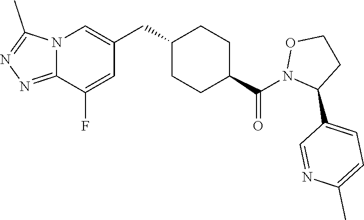 [SMILES: Cc1ccc([C@@H]2CCON2C(=O)[C@H]2CC[C@H](Cc3cc(F)c4nnc(C)n4c3)CC2)cn1] | (600 MHz, DMSO-d6) δ = 8.34 (d, J = 2.2 Hz, 1H), 8.04 (s, 1H), 7.52 (dd, J = 8.1, 2.4 Hz, 1H), 7.29- 7.13 (m, 2H), 5.35-5.21 (m, 1H), 4.26 (td, J = 7.6, 2.6 Hz, 1H), 3.90-3.80 (m, 1H), 2.94-2.80 (m, 1H), 2.74-2.61 (m, 4H), 2.49-2.47 (m, 2H, below solvent signal), 2.43 (s, 3H), 2.25-2.14 (m, 1H), 1.89-1.78 (m, 1H), 1.74-1.66 (m, 3H), 1.64-1.54 (m, 1H), 1.37- 1.26 (m, 2H), 1.13-0.96 (m, 2H) LC/MS: m/z 438.2 [M + H]⁺, Rt (I = 220 nm): 1.220 min (LC/MS method A), calc. m/z: 438.2. |
| 365 | 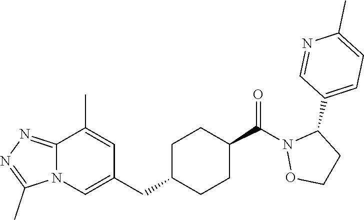 [SMILES: Cc1ccc([C@@H]2CCON2C(=O)[C@H]2CC[C@H](Cc3cc(C)c4nnc(C)n4c3)CC2)cn1] | ¹H NMR (600 MHz, DMSO-d6) δ = 8.34 (d, J = 2.2 Hz, 1H), 7.95 (s, 1H), 7.52 (dd, J = 8.1, 2.4 Hz, 1H), 7.21 (d, J = 8.1 Hz, 1H), 7.02 (s, 1H), 5.34-5.23 (m, 1H), 4.26 (td, J = 7.7, 2.7 Hz, 1H), 3.90-3.78 (m, 1H), 2.88-2.81 (m, 1H), 2.68-2.62 (m, 4H), 2.48 (s, 3H), 2.45 (d, J = 7.2 Hz, 2H), 2.43 (s, 3H), 2.23-2.15 (m, 1H), 1.86-1.81 (m, 1H), 1.75-1.66 (m, 3H), 1.61- 1.53 (m, 1H), 1.35-1.22 (m, 3H), 1.10-0.98 (m, 2H). LC/MS: m/z 434.2 [M + H]⁺, Rt (I = 220 nm): 1.160 min (LC/MS method A), calc. m/z: 434.2. |
| 366 | 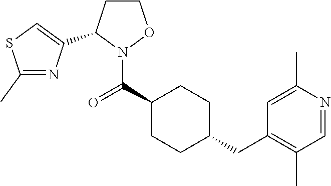 [SMILES: Cc1cc(C[C@H]2CC[C@H](C(=O)N3OCC[C@H]3c3csc(C)n3)CC2)c(C)cn1] | ¹H NMR (600 MHz, DMSO-d6) δ = 8.16 (s, 1H), 7.18 (br s, 1H), 6.96 (s, 1H), 5.40-5.31 (m, 1H), 4.23 (td, J = 7.7, 3.9 Hz, 1H), 3.89-3.79 (m, 1H), 2.73-2.68 (m, 1H), 2.62 (s, 4H), 2.45-2.42 (m, 2H), 2.40-2.36 (m, 4H), 2.18 (s, 3H), 1.83-1.77 (m, 1H), 1.69-1.62 (m, 3H), 1.56-1.48 (m, 1H), 1.34-1.24 (m, 2H), 1.12- 1.02 (m, 2H). LC/MS: m/z 400.2 [M + H]⁺, Rt (I = 220 nm): 1.260 min (LC/MS method A), calc. m/z: 400.2. |
| 367 | 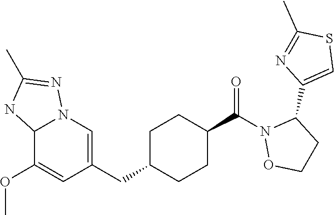 [SMILES: COC1=CC(C[C@H]2CC[C@H](C(=O)N3OCC[C@H]3c3csc(C)n3)CC2)=CN2N=C(C)NC12] | ¹H NMR (600 MHz, DMSO-d6) δ = 8.20 (d, J = 0.9 Hz, 1H), 7.18 (br s, 1H), 6.90 (d, J = 1.1 Hz, 1H), 5.41- 5.32 (m, 1H), 4.27-4.19 (m, 1H), 3.95 (s, 3H), 3.89- 3.81 (m, 1H), 2.73-2.67 (m, 1H), 2.67-2.61 (m, 4H), 2.52-2.51 (m, 2H), 2.42-2.36 (m, 4H), 1.85- 1.79 (m, 1H), 1.73-1.66 (m, 3H), 1.66-1.57 (m, 1H), 1.37-1.27 (m, 2H), 1.08-0.96 (m, 2H). LC/MS: m/z 456.2 [M + H]⁺, Rt (I = 220 nm): 1.830 min (LC/MS method A), calc. m/z: 456.2. |
| 368 | 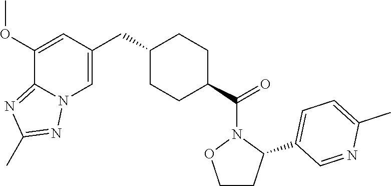 [SMILES: COc1cc(C[C@H]2CC[C@H](C(=O)N3OCC[C@H]3c3ccc(C)nc3)CC2)cn2nc(C)nc12] | ¹H NMR (400 MHz, MeOD-d4) δ ppm = = 8.34 (d, J = 1.7 Hz, 1H), 8.06 (s, 1H), 7.65 (dd, J = 8.1, 2.1 Hz, 1H), 7.28 (d, J = 7.9 Hz, 1H), 6.93 (s, 1H), 5.39-5.32 (m, 1H), 4.32 (dt, J = 7.8, 3.1 Hz, 1H), 4.04-3.93 (m, 4H), 2.98-2.88 (m, 1H), 2.88-2.73 (m, 1H), 2.58 (br d, J = 7.0 Hz, 2H), 2.53-2.45 (m, 6H), 2.38-2.25 (m, 1H), 1.99-1.88 (m, 1H), 1.86-1.74 (m, 3H), 1.71- 1.57 (m, 1H), 1.52-1.35 (m, 2H), 1.21-1.02 (m, 2H). LC/MS: m/z 450.2 [M + H]⁺, Rt (I = 220 nm): 0.405 min (LC/MS method Q), calc. m/z: 450.2. |
| 369 | 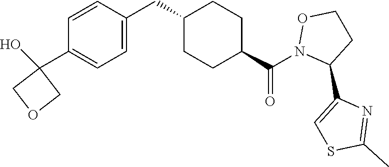 [SMILES: Cc1nc([C@@H]2CCON2C(=O)[C@H]2CC[C@H](Cc3ccc(C4(O)COC4)cc3)CC2)cs1] | ¹H NMR (400 MHz, DMSO-d6) δ = 7.54-7.45 (m, 2H), 7.22-7.14 (m, 3H), 6.22 (s, 1H), 5.38-5.31 (m, 1H), 4.74 (d, J = 6.8 Hz, 2H), 4.67 (d, J = 6.8 Hz, 2H), 4.22 (td, J = 7.6, 4.0 Hz, 1H), 3.87-3.80 (m, 1H), 2.74-2.67 (m, 1H), 2.66-2.60 (m, 4H), 2.47 (d, J = 7.0 Hz, 2H), 2.44-2.36 (m, 1H), 1.83-1.78 (m, 1H), 1.71-1.63 (m, 3H), 1.52-1.43 (m, 1H), 1.32-1.23 (m, 2H), 1.08-0.95 (m, 2H). LC/MS: m/z 443.2 [M + H]⁺, Rt (I = 220 nm): 1.985 min (LC/MS method A), calc. m/z: 443.2. |
| 370 | 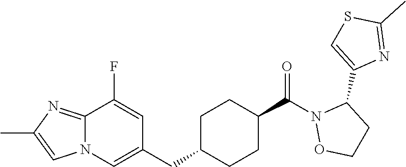 | ¹H NMR (600 MHz, DMSO-d6) δ = 8.11 (s, 1H), 7.71 (d, J = 2.6 Hz, 1H), 7.18 (br s, 1H), 6.97 (dd, J = 12.3, 0.9 Hz, 1H), 5.37-5.32 (m, 1H), 4.22 (td, J = 7.7, 3.8 Hz, 1H), 3.87-3.81 (m, 1H), 2.73-2.67 (m, 1H), 2.62 (s, 4H), 2.46-2.42 (m, 2H), 2.42-2.35 (m, 1H), 2.32 (d, J = 0.6 Hz, 3H), 1.85-1.79 (m, 1H), 1.72-1.65 (m, 3H), 1.60-1.50 (m, 1H), 1.36-1.26 (m, 2H), 1.07- 0.94 (m, 2H). LC/MS: m/z 443.2 [M + H]⁺, Rt (I = 220 nm): 1.360 min (LC/MS method A), calc. m/z: 443.2. |
| 371 | 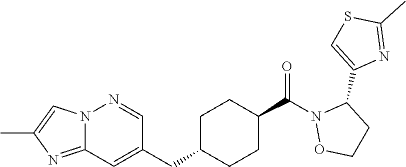 | ¹H NMR (600 MHz, DMSO-d6) δ = 8.30 (d, J = 2.0 Hz, 1H), 7.94 (s, 1H), 7.71 (d, J = 1.5 Hz, 1H), 7.18 (br s, 1H), 5.38-5.32 (m, 1H), 4.22 (td, J = 7.7, 3.9 Hz, 1H), 3.84 (q, J = 7.9 Hz, 1H), 2.69 (br d, J = 3.1 Hz, 1H), 2.67-2.61 (m, 4H), 2.55 (d, J = 7.0 Hz, 2H), 2.42-2.36 (m, 1H), 2.36-2.35 (m, 3H), 1.86-1.80 (m, 1H), 1.76-1.65 (m, 3H), 1.64-1.54 (m, 1H), 1.37- 1.26 (m, 2H), 1.09-0.98 (m, 2H). LC/MS: m/z 426.2 [M + H]⁺, Rt (I = 220 nm): 1.300 min (LC/MS method A), calc. m/z: 426.2. |
| 372 | 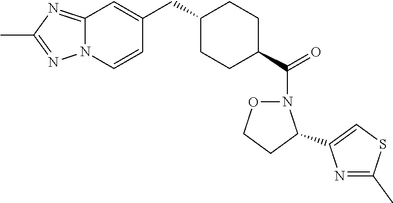 | ¹H NMR (600 MHz, DMSO-d6) δ = 8.69 (d, J = 7.0 Hz, 1H), 7.44 (s, 1H), 7.18 (br s, 1H), 6.95 (dd, J = 7.0, 1.7 Hz, 1H), 5.43-5.27 (m, 1H), 4.22 (td, J = 7.7, 4.0 Hz, 1H), 3.84 (q, J = 8.1 Hz, 1H), 2.73-2.67 (m, 1H), 2.66-2.61 (m, 4H), 2.59 (d, J = 7.2 Hz, 2H), 2.43 (s, 3H), 2.42-2.35 (m, 1H), 1.86-1.79 (m, 1H), 1.73-1.57 (m, 4H), 1.37-1.22 (m, 2H), 1.11-0.98 (m, 2H). LC/MS: m/z 426.2 [M + H]⁺, Rt (I = 220 nm): 1.700 min (LC/MS method A), calc. m/z: 426.2. |
| 373 | 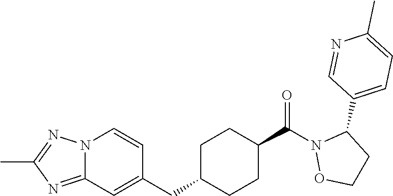 [SMILES: Cc1ccc([C@@H]2CCON2C(=O)[C@H]2CC[C@H](Cc3ccn4nc(C)nc4c3)CC2)cn1] | ¹H NMR (600 MHz, DMSO-d6) δ = 8.69 (d, J = 7.2 Hz, 1H), 8.34 (d, J = 2.2 Hz, 1H), 7.52 (dd, J = 8.1, 2.4 Hz, 1H), 7.44 (s, 1H), 7.21 (d, J = 7.9 Hz, 1H), 6.95 (dd, J = 7.0, 1.8 Hz, 1H), 5.33-5.26 (m, 1H), 4.30-4.22 (m, 1H), 3.90-3.80 (m, 1H), 2.88-2.81 (m, 1H), 2.69-2.63 (m, 1H), 2.62-2.57 (m, 2H), 2.46-2.41 (m, 6H), 2.24-2.16 (m, 1H), 1.85-1.81 (m, 1H), 1.72-1.65 (m, 3H), 1.64-1.57 (m, 1H), 1.34-1.25 (m, 2H), 1.11-0.99 (m, 2H). LC/MS: m/z 210.7 [M + H/2]²⁺, Rt (I = 220 nm): 1.300 min (LC/MS method A), calc. m/z: 420.5. |
| 374 | 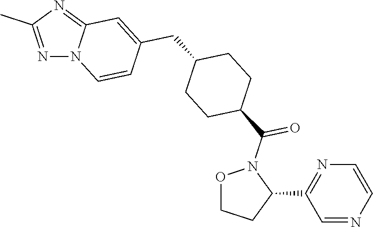 [SMILES: Cc1nc2cc(C[C@H]3CC[C@H](C(=O)N4OCC[C@H]4c4cnccn4)CC3)ccn2n1] | ¹H NMR (600 MHz, DMSO-d6) δ = 8.72-8.66 (m, 1H), 8.60 (dd, J = 2.4, 1.5 Hz, 1H), 8.58-8.54 (m, 2H), 7.47-7.41 (m, 1H), 6.95 (dd, J = 7.0, 1.7 Hz, 1H), 5.39 (dd, J = 8.7, 6.1 Hz, 1H), 4.30 (td, J = 7.7, 3.6 Hz, 1H), 3.96-3.86 (m, 1H), 2.84-2.76 (m, 1H), 2.72-2.63 (m, 1H), 2.60 (d, J = 7.0 Hz, 2H), 2.49- 2.45 (m, 1H), 2.43 (s, 3H), 1.87-1.81 (m, 1H), 1.75- 1.66 (m, 3H), 1.65-1.57 (m, 1H), 1.36-1.23 (m, 2H), 1.12-1.00 (m, 2H). LC/MS: m/z 407.2 [M + H]⁺, Rt (I = 220 nm): 0.420 min (LC/MS method Q), calc. m/z: 407.2. |
| 375 | 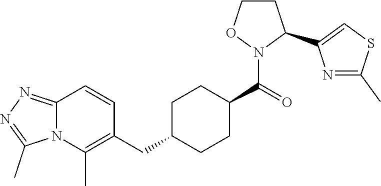 [SMILES: Cc1nc([C@@H]2CCON2C(=O)[C@H]2CC[C@H](Cc3ccc4nnc(C)n4c3C)CC2)cs1] | ¹H NMR (600 MHz, DMSO-d6) δ = 7.41 (d, J = 9.2 Hz, 1H), 7.18 (br s, 1H), 7.06 (d, J = 9.4 Hz, 1H), 5.42- 5.31 (m, 1H), 4.27-4.14 (m, 1H), 3.91-3.77 (m, 1H), 2.95 (s, 3H), 2.77 (s, 3H), 2.74-2.64 (m, 2H), 2.62 (s, 3H), 2.52-2.50 (m, 2H, below solvent signal), 2.43-2.35 (m, 1H), 1.85-1.79 (m, 1H), 1.74- 1.62 (m, 3H), 1.49-1.41 (m, 1H), 1.33-1.23 (m, 2H), 1.15-1.01 (m, 2H). LC/MS: m/z 439.5 [M + H]⁺, Rt (I = 220 nm): 1.530 min (LC/MS method A), calc. m/z: 439.5. |
| 376 | 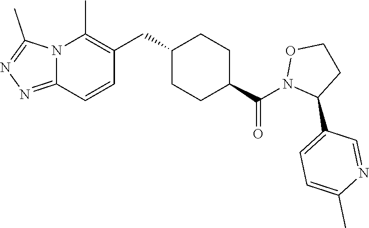 [SMILES: Cc1ccc([C@@H]2CCON2C(=O)[C@H]2CC[C@H](Cc3ccc4nnc(C)n4c3C)CC2)cn1] | ¹H NMR (600 MHz, DMSO-d6) δ = 8.34 (d, J = 2.0 Hz, 1H), 7.52 (dd, J = 8.0, 2.3 Hz, 1H), 7.41 (d, J = 9.4 Hz, 1H), 7.21 (d, J = 8.1 Hz, 1H), 7.06 (d, J = 9.4 Hz, 1H), 5.33-5.23 (m, 1H), 4.32-4.19 (m, 1H), 3.92- 3.78 (m, 1H), 2.95 (s, 3H), 2.88-2.81 (m, 1H), 2.77 (s, 3H), 2.71-2.64 (m, 1H), 2.50-2.48 (m, 2H, below solvent signal), 2.43 (s, 3H), 2.24-2.15 (m, 1H), 1.87- 1.77 (m, 1H), 1.75-1.62 (m, 3H), 1.49-1.40 (m, 1H), 1.32-1.20 (m, 2H), 1.17-1.04 (m, 2H). LC/MS: m/z 434.2 [M + H]⁺, Rt (I = 220 nm): 1.140 min (LC/MS method A), calc. m/z: 434.2. |
| 377 | 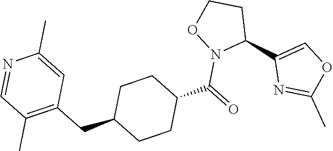 [SMILES: Cc1cc(C[C@H]2CC[C@H](C(=O)N3OCC[C@H]3c3coc(C)n3)CC2)c(C)cn1] | ¹H NMR (400 MHz, MeOD-d4) δ ppm = 8.16 (s, 1H), 7.66 (s, 1H), 7.09 (s, 1H), 5.31 (br dd, J = 8.3, 5.4 Hz, 1H), 4.32 (td, J = 7.7, 4.2 Hz, 1H), 4.03-3.93 (m, 1H), 2.86-2.67 (m, 2H), 2.57 (d, J = 7.3 Hz, 2H), 2.55-2.45 (m, 4H), 2.45-2.41 (m, 3H), 2.29 (s, 3H), 1.95-1.87 (m, 1H), 1.84-1.73 (m, 3H), 1.68-1.56 (m, 1H), 1.49-1.32 (m, 2H), 1.24-1.07 (m, 2H). LC/MS: m/z 384.2 [M + H]⁺, Rt (I = 220 nm): 0.929 min (LC/MS method S), calc. m/z: 384.2. |
| 378 | 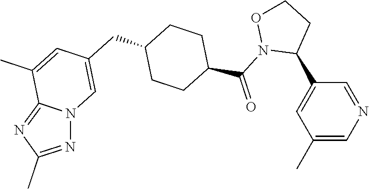 [SMILES: Cc1cncc([C@@H]2CCON2C(=O)[C@H]2CC[C@H](Cc3cc(C)c4nc(C)nn4c3)CC2)c1] | ¹H NMR (400 MHz, DMSO-d6) δ = 8.45 (s, 1H), 8.34- 8.26 (m, 2H), 7.48 (s, 1H), 7.28 (s, 1H), 5.37-5.25 (m, 1H), 4.27 (td, J = 7.8, 2.9 Hz, 1H), 3.92-3.78 (m, 1H), 2.92-2.81 (m, 1H), 2.73-2.61 (m, 1H), 2.50- 2.49 (m, 2H), 2.47 (s, 3H), 2.44 (s, 3H), 2.31-2.27 (m, 3H), 2.26-2.15 (m, 1H), 1.90-1.80 (m, 1H), 1.74- 1.64 (m, 3H), 1.62-1.47 (m, 1H), 1.37-1.20 (m, 2H), 1.10-0.96 (m, 2H). LC/MS: m/z 434.2 [M + H]⁺, Rt (I = 220 nm): 0.423 min (LC/MS method Q), calc. m/z: 434.2. |
| 379 | 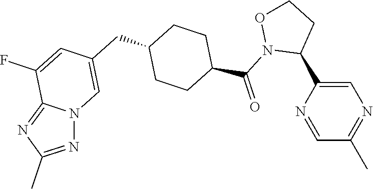 [SMILES: Cc1cnc([C@@H]2CCON2C(=O)[C@H]2CC[C@H](Cc3cc(F)c4nc(C)nn4c3)CC2)cn1] | ¹H NMR (400 MHz, DMSO-d6) δ = 8.58 (s, 1H), 8.49 (s, 1H), 8.43 (s, 1H), 7.54-7.46 (m, 1H), 5.36 (dd, J = 8.6, 6.3 Hz, 1H), 4.29 (td, J = 7.5, 3.7 Hz, 1H), 3.97- 3.88 (m, 1H), 2.83-2.73 (m, 1H), 2.70-2.60 (m, 1H), 2.58-2.53 (m, 2H), 2.49-2.42 (m, 7H), 1.86- 1.77 (m, 1H), 1.76-1.64 (m, 3H), 1.63-1.51 (m, 1H), 1.37-1.21 (m, 2H), 1.11-0.95 (m, 2H). LC/MS: m/z 439.2 [M + H]⁺, Rt (I = 220 nm): 0.827 min (LC/MS method R), calc. m/z: 439.2. |
| 380 | 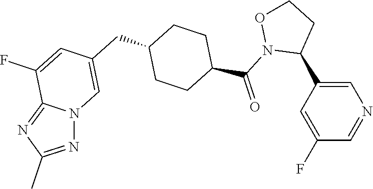 [SMILES: Cc1nc2c(F)cc(C[C@H]3CC[C@H](C(=O)N4OCC[C@H]4c4cncc(F)c4)CC3)cn2n1] | ¹H NMR (400 MHz, DMSO-d6) δ = 8.57 (s, 1H), 8.48 (d, J = 2.4 Hz, 1H), 8.38 (s, 1H), 7.57 (br d, J = 9.8 Hz, 1H), 7.49 (d, J = 11.7 Hz, 1H), 5.46-5.31 (m, 1H), 4.27 (td, J = 7.8, 2.9 Hz, 1H), 3.98-3.79 (m, 1H), 2.96-2.82 (m, 1H), 2.74-2.60 (m, 1H), 2.55 (br d, J = 6.8 Hz, 2H), 2.47 (s, 3H), 2.35-2.18 (m, 1H), 1.94-1.80 (m, 1H), 1.77-1.63 (m, 3H), 1.63-1.51 (m, 1H), 1.40-1.18 (m, 2H), 1.13-0.92 (m, 2H). LC/MS: m/z 442.1 [M + H]⁺, Rt (I = 220 nm): 0.478 min (LC/MS method Q), calc. m/z: 442.2. |
| 381 | 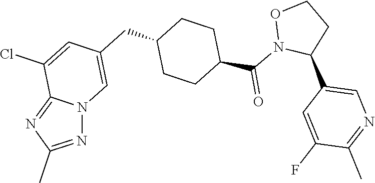 [SMILES: Cc1nc2c(Cl)cc(C[C@H]3CC[C@H](C(=O)N4OCC[C@H]4c4cnc(C)c(F)c4)CC3)cn2n1] | ¹H NMR (400 MHz, DMSO-d6) δ = 8.68 (s, 1H), 8.22 (s, 1H), 7.73 (d, J = 1.0 Hz, 1H), 7.46 (dd, J = 10.5, 1.5 Hz, 1H), 5.39-5.30 (m, 1H), 4.26 (td, J = 7.7, 3.0 Hz, 1H), 3.94-3.78 (m, 1H), 2.91-2.79 (m, 1H), 2.71- 2.59 (m, 1H), 2.54 (d, J = 7.0 Hz, 2H), 2.47 (s, 3H), 2.41 (d, J = 2.9 Hz, 3H), 2.30-2.14 (m, 1H), 1.90- 1.80 (m, 1H), 1.73-1.64 (m, 3H), 1.62-1.53 (m, 1H), 1.37-1.19 (m, 2H), 1.13-0.93 (m, 2H). LC/MS: m/z 472.1 [M + H]⁺, Rt (I = 220 nm): 0.923 min (LC/MS method R), calc. m/z: 472.2. |
| 382 | 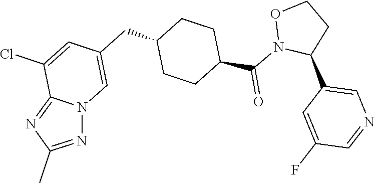 [SMILES: Cc1nc2c(Cl)cc(C[C@H]3CC[C@H](C(=O)N4OCC[C@H]4c4cncc(F)c4)CC3)cn2n1] | ¹H NMR (400 MHz, DMSO-d6) δ = 8.69 (s, 1H), 8.49 (d, J = 2.4 Hz, 1H), 8.39 (s, 1H), 7.74 (d, J = 1.5 Hz, 1H), 7.58 (dt, J = 9.5, 2.3 Hz, 1H), 5.39 (dd, J = 8.3, 6.4 Hz, 1H), 4.28 (dt, J = 7.8, 2.9 Hz, 1H), 3.95-3.82 (m, 1H), 2.95-2.83 (m, 1H), 2.74-2.62 (m, 1H), 2.56 (d, J = 7.3 Hz, 2H), 2.48 (s, 3H), 2.33-2.20 (m, 1H), 1.94-1.83 (m, 1H), 1.75-1.65 (m, 3H), 1.64-1.53 (m, 1H), 1.37-1.19 (m, 2H), 1.11-0.93 (m, 2H). LC/MS: m/z 458.1 [M + H]⁺, Rt (I = 220 nm): 0.896 min (LC/MS method R), calc. m/z: 458.2. |
| 383 | 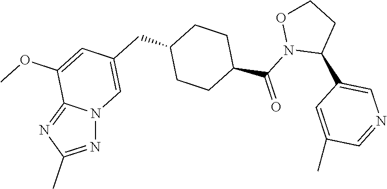 | ¹H NMR (400 MHz, MeOD-d4) δ ppm = 8.30 (m, 2H), 8.08 (s, 1H), 7.61 (s, 1H), 6.95 (s, 1H), 5.44-5.28 (m, 1H), 4.35 (td, J = 7.8, 3.1 Hz, 1H), 4.08-3.93 (m, 4H), 3.02-2.90 (m, 1H), 2.90-2.76 (m, 1H), 2.60 (d, J = 7.1 Hz, 2H), 2.52 (s, 3H), 2.42-2.27 (m, 4H), 2.01-1.92 (m, 1H), 1.89-1.74 (m, 3H), 1.74-1.59 (m, 1H), 1.53-1.37 (m, 2H), 1.22-1.04 (m, 2H). LC/MS: m/z 434.2 [M + H]⁺, Rt (I = 220 nm): 0.423 min (LC/MS method Q), calc. m/z: 434.2. |
| 384 | 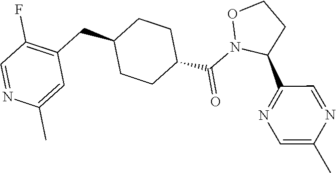 [SMILES: Cc1cnc([C@@H]2CCON2C(=O)[C@H]2CC[C@H](Cc3cc(C)ncc3F)CC2)cn1] | ¹H NMR (400 MHz, MeOD-d4) δ ppm = 8.49 (s, 1H), 8.47-8.44 (m, 1H), 8.20 (d, J = 1.8 Hz, 1H), 7.20 (d, J = 6.0 Hz, 1H), 5.43 (dd, J = 8.6, 6.1 Hz, 1H), 4.37 (td, J = 7.6, 4.0 Hz, 1H), 4.10-4.00 (m, 1H), 2.96- 2.73 (m, 2H), 2.53-2.51 (m, 6H), 2.49 (s, 3H), 1.98- 1.88 (m, 1H), 1.85-1.72 (m, 3H), 1.70-1.56 (m, 1H), 1.50-1.31 (m, 2H), 1.22-1.08 (m, 2H). LC/MS: m/z 399.2 [M + H]⁺, Rt (I = 220 nm): 0.415 min (LC/MS method Q), calc. m/z: 399.2. |
| 385 | 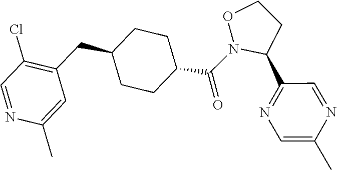 [SMILES: Cc1cnc([C@@H]2CCON2C(=O)[C@H]2CC[C@H](Cc3cc(C)ncc3Cl)CC2)cn1] | ¹H NMR (400 MHz, MeOD-d4) δ ppm = 8.47 (s, 1H), 8.44 (s, 1H), 8.33 (s, 1H), 7.20 (s, 1H), 5.41 (dd, J = 8.6, 6.1 Hz, 1H), 4.35 (td, J = 7.6, 3.9 Hz, 1H), 4.10- 3.98 (m, 1H), 2.91-2.73 (m, 2H), 2.70-2.63 (m, 2H), 2.63-2.51 (m, 4H), 2.48 (s, 3H), 1.97-1.88 (m, 1H), 1.86-1.61 (m, 4H), 1.47-1.27 (m, 2H), 1.24-1.09 (m, 2H). LC/MS: m/z 415.1 [M + H]⁺, Rt (I = 220 nm): 0.445 min (LC/MS method Q), calc. m/z: 415.2. |
| 386 | 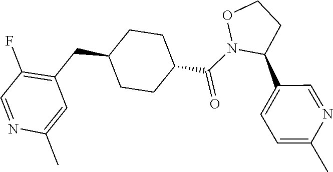 [SMILES: Cc1ccc([C@@H]2CCON2C(=O)[C@H]2CC[C@H](Cc3cc(C)ncc3F)CC2)cn1] | ¹H NMR (400 MHz, MeOD-d4) δ ppm = 8.36 (d, J = 1.8 Hz, 1H), 8.20 (d, J = 1.8 Hz, 1H), 7.67 (dd, J = 8.1, 2.2 Hz, 1H), 7.30 (d, J = 8.1 Hz, 1H), 7.20 (d, J = 6.1 Hz, 1H), 5.43-5.34 (m, 1H), 4.34 (td, J = 7.7, 3.2 Hz, 1H), 4.04-3.92 (m, 1H), 3.01-2.89 (m, 1H), 2.87- 2.75 (m, 1H), 2.60 (d, J = 7.1 Hz, 2H), 2.52 (s, 3H), 2.49 (s, 3H), 2.41-2.26 (m, 1H), 2.01-1.91 (m, 1H), 1.84-1.73 (m, 3H), 1.70-1.59 (m, 1H), 1.51-1.34 (m, 2H), 1.23-1.07 (m, 2H). LC/MS: m/z 397.2 [M + H]⁺, Rt (I = 220 nm): 0.381 min (LC/MS method Q), calc. m/z: 397.2. |
| 387 | 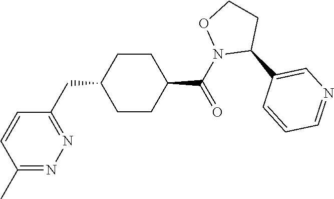 | ¹H NMR (400 MHz, MeOD-d4) δ ppm = 8.34 (d, J = 1.8 Hz, 1H), 7.65 (dd, J = 8.1, 2.2 Hz, 1H), 7.57-7.49 (m, 2H), 7.28 (d, J = 8.1 Hz, 1H), 5.41-5.31 (m, 1H), 4.32 (td, J = 7.8, 3.2 Hz, 1H), 4.30-3.92 (m, 1H), 2.99-2.88 (m, 1H), 2.87-2.72 (m, 3H), 2.64 (s, 3H), 2.50 (s, 3H), 2.38-2.26 (m, 1H), 1.99-1.89 (m, 1H), 1.84-1.68 (m, 4H), 1.51-1.32 (m, 2H), 1.25-1.07 (m, 2H). LC/MS: m/z 381.1 [M + H]⁺, Rt (I = 220 nm): 0.355 min (LC/MS method Q), calc. m/z: 381.2. |
| 388 | 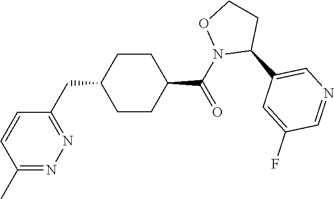 | ¹H NMR (400 MHz, MeOD-d4) δ ppm = 8.25-8.11 (m, 1H), 7.58-7.49 (m, 2H), 7.46 (br d, J = 10.3 Hz, 1H), 5.43-5.34 (m, 1H), 4.32 (td, J = 7.9, 2.7 Hz, 1H), 4.02-3.92 (m, 1H), 3.00-2.89 (m, 1H), 2.87- 2.74 (m, 3H), 2.64 (s, 3H), 2.47 (d, J = 2.6 Hz, 3H), 2.40-2.26 (m, 1H), 2.01-1.90 (m, 1H), 1.84-1.69 (m, 4H), 1.52-1.33 (m, 2H), 1.28-1.05 (m, 3H) LC/MS: m/z 399.2 [M + H]⁺, Rt (I = 220 nm): 0.407 min (LC/MS method Q), calc. m/z: 399.2. |
| 389 | 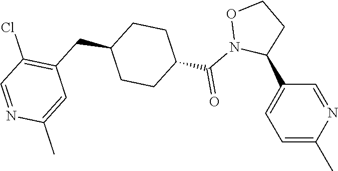 [SMILES: Cc1ccc([C@@H]2CCON2C(=O)[C@H]2CC[C@H](Cc3cc(C)ncc3Cl)CC2)cn1] | ¹H NMR (400 MHz, MeOD-d4) δ ppm = 8.36-8.29 (m, 2H), 7.69-7.62 (m, 1H), 7.28 (d, J = 8.1 Hz, 1H), 7.20 (s, 1H), 5.40-5.29 (m, 1H), 4.32 (td, J = 7.7, 2.9 Hz, 1H), 4.02-3.89 (m, 1H), 2.98-2.87 (m, 1H), 2.87- 2.73 (m, 1H), 2.66 (d, J = 7.0 Hz, 2H), 2.50 (s, 3H), 2.48 (s, 3H), 2.38-2.26 (m, 1H), 1.98-1.88 (m, 1H), 1.83-1.59 (m, 4H), 1.48-1.28 (m, 2H), 1.25-1.06 (m, 2H). LC/MS: m/z 414.2 [M + H]⁺, Rt (I = 220 nm): 0.977 min (LC/MS method S), calc. m/z: 414.2. |
| 390 | 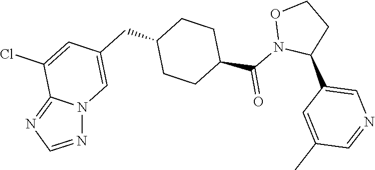 [SMILES: Cc1cncc([C@@H]2CCON2C(=O)[C@H]2CC[C@H](Cc3cc(Cl)c4ncnn4c3)CC2)c1] | ¹H NMR (400 MHz, MeOD-d4) δ ppm = 8.51 (s, 1H), 8.40 (s, 1H), 8.32-8.25 (m, 2H), 7.59 (s, 1H), 7.45 (d, J = 10.3 Hz, 1H), 5.41-5.31 (m, 1H), 4.33 (td, J = 7.7, 3.2 Hz, 1H), 4.04-3.88 (m, 1H), 3.00-2.90 (m, 1H), 2.89-2.75 (m, 1H), 2.65 (d, J = 6.8 Hz, 2H), 2.40-2.24 (m, 4H), 2.02-1.91 (m, 1H), 1.88-1.75 (m, 3H), 1.73-1.58 (m, 1H), 1.44 (br s, 2H), 1.23- 1.02 (m, 2H). LC/MS: m/z 414.2 [M + H]⁺, Rt (I = 220 nm): 0.977 min (LC/MS method S), calc. m/z: 414.2. |
| 391 | 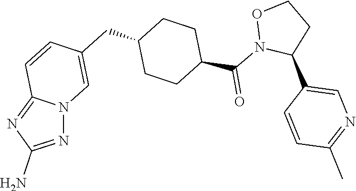 [SMILES: Cc1ccc([C@@H]2CCON2C(=O)[C@H]2CC[C@H](Cc3ccc4nc(N)nn4c3)CC2)cn1] | ¹H NMR (400 MHz, MeOD-d4) δ ppm = 8.36 (s, 1H), 8.24 (s, 1H), 7.70-7.64 (m, 1H), 7.44-7.38 (m, 1H), 7.35-7.23 (m, 2H), 5.42-5.33 (m, 1H), 4.40-4.28 (m, 1H), 4.05-3.90 (m, 1H), 3.00-2.88 (m, 1H), 2.88- 2.74 (m, 1H), 2.57 (d, J = 7.1 Hz, 2H), 2.52 (s, 3H), 2.40-2.25 (m, 1H), 2.01-1.91 (m, 1H), 1.86-1.76 (m, 3H), 1.68-1.53 (m, 1H), 1.53-1.34 (m, 2H), 1.20- 1.01 (m, 2H). LC/MS: m/z 421.3 [M + H]⁺, Rt (I = 220 nm): 0.462 min (LC/MS method S), calc. m/z: 421.2. |
| 392 | 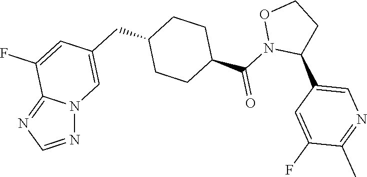 [SMILES: Cc1ncc([C@@H]2CCON2C(=O)[C@H]2CC[C@H](Cc3cc(F)c4ncnn4c3)CC2)cc1F] | ¹H NMR (400 MHz, MeOD-d4) δ ppm = 8.51 (s, 1H), 8.40 (s, 1H), 8.21 (s, 1H), 7.53-7.38 (m, 2H), 5.46- 5.33 (m, 1H), 4.33 (td, J = 7.7, 3.2 Hz, 1H), 4.05- 3.86 (m, 1H), 2.99-2.89 (m, 1H), 2.86-2.74 (m, 1H), 2.65 (d, J = 6.8 Hz, 2H), 2.47 (d, J = 2.4 Hz, 3H), 2.41- 2.26 (m, 1H), 2.04-1.89 (m, 1H), 1.86-1.74 (m, 3H), 1.74-1.61 (m, 1H), 1.54-1.33 (m, 2H), 1.24- 1.02 (m, 2H). LC/MS: m/z 442.1 [M + H]⁺, Rt (I = 220 nm): 0.891 min (LC/MS method R), calc. m/z: 442.2. |
| 393 | 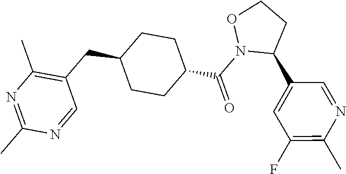 | ¹H NMR (400 MHz, MeOD-d4) δ ppm = 8.34 (s, 1H), 8.23 (s, 1H), 7.48 (dd, J = 10.1, 2.7 Hz, 1H), 5.45- 5.37 (m, 1H), 4.35 (td, J = 7.9, 3.1 Hz, 1H), 4.04- 3.93 (m, 1H), 3.02-2.92 (m, 1H), 2.91-2.74 (m, 1H), 2.62 (s, 3H), 2.58 (d, J = 7.1 Hz, 2H), 2.52 (s, 3H), 2.49 (d, J = 2.8 Hz, 3H), 2.41-2.30 (m, 1H), 2.03- 1.93 (m, 1H), 1.85-1.73 (m, 3H), 1.63-1.51 (m, 1H), 1.50-1.35 (m, 2H), 1.26-1.09 (m, 2H). LC/MS: m/z 413.3 [M + H]⁺, Rt (I = 220 nm): 0.901 min (LC/MS method S), calc. m/z: 413.2. |
| 394 | 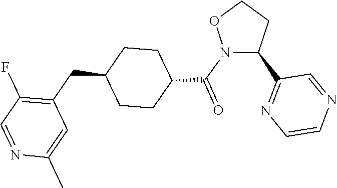 | ¹H NMR (400 MHz, MeOD-d4) δ ppm = 8.66-8.57 (m, 2H), 8.53 (d, J = 2.5 Hz, 1H), 8.28-8.13 (m, 1H), 7.21 (d, J = 6.1 Hz, 1H), 5.47 (br dd, J = 8.5, 6.3 Hz, 1H), 4.38 (td, J = 7.6, 4.0 Hz, 1H), 4.13-4.00 (m, 1H), 2.98-2.76 (m, 2H), 2.67-2.55 (m, 3H), 2.50 (s, 3H), 2.03-1.90 (m, 1H), 1.88-1.73 (m, 3H), 1.72- 1.58 (m, 1H), 1.52-1.32 (m, 2H), 1.25-1.06 (m, 2H). LC/MS: m/z 434.2 [M + H]⁺, Rt (I = 220 nm): 0.413 min (LC/MS method Q), calc. m/z: 434.2. |
| 395 | 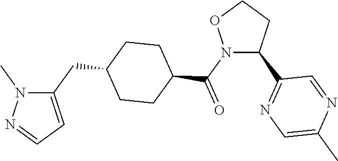 | ¹H NMR (400 MHz, Chloroform-d) δ ppm = 8.56- 8.50 (m, 1H), 8.40 (s, 1H), 7.39 (d, J = 1.6 Hz, 1H), 6.01 (d, J = 1.6 Hz, 1H), 5.48 (dd, J = 8.6, 5.9 Hz, 1H), 4.32 (dt, J = 7.7, 3.8 Hz, 1H), 4.00-3.89 (m, 1H), 3.80 (s, 3H), 2.84-2.61 (m, 3H), 2.55 (s, 3H), 2.52 (d, J = 7.0 Hz, 2H), 2.03-1.90 (m, 1H), 1.88- 1.75 (m, 3H), 1.66-1.41 (m, 3H), 1.16-0.97 (m, 2H). LC/MS: m/z 370.3 [M + H]⁺, Rt (I = 220 nm): 0.517 min (LC/MS method Q), calc. m/z: 370.2. |
| 396 | 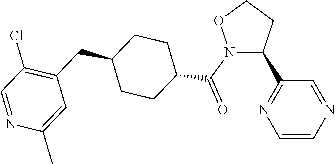 [SMILES: Cc1cc(C[C@H]2CC[C@H](C(=O)N3OCC[C@H]3c3cnccn3)CC2)c(Cl)cn1] | ¹H NMR (400 MHz, MeOD-d4) δ ppm = 8.64-8.55 (m, 2H), 8.51 (s, 1H), 8.33 (s, 1H), 7.20 (s, 1H), 5.45 (br dd, J = 8.2, 6.3 Hz, 1H), 4.36 (dt, J = 7.4, 4.1 Hz, 1H), 4.09-4.01 (m, 1H), 2.95-2.75 (m, 2H), 2.70- 2.53 (m, 3H), 2.48 (s, 3H), 2.00-1.89 (m, 1H), 1.88- 1.61 (m, 4H), 1.49-1.28 (m, 2H), 1.27-1.07 (m, 2H). LC/MS: m/z 401.2 [M + H]⁺, Rt (I = 220 nm): 0.441 min (LC/MS method Q), calc. m/z: 401.2. |
| 397 | 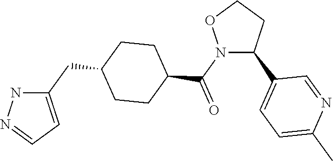 | ¹H NMR (400 MHz, Chloroform-d) δ ppm = 8.44 (br s, 1H), 7.49 (dd, J = 8.1, 2.1 Hz, 1H), 7.38 (d, J = 1.5 Hz, 1H), 7.11 (br d, J = 7.9 Hz, 1H), 5.99 (d, J = 1.2 Hz, 1H), 5.38 (dd, J = 8.6, 6.2 Hz, 1H), 4.26 (td, J = 7.8, 3.4 Hz, 1H), 3.95-3.83 (m, 1H), 3.78 (s, 3H), 2.88-2.78 (m, 1H), 2.78-2.67 (m, 1H), 2.57-2.47 (m, 5H), 2.41-2.26 (m, 1H), 2.00-1.92 (m, 1H), 1.89- 1.72 (m, 3H), 1.66-1.40 (m, 3H), 1.16-0.99 (m, 2H). LC/MS: m/z 369.3 [M + H]⁺, Rt (I = 220 nm): 0.477 min (LC/MS method Q), calc. m/z: 369.2. |
| 398 | 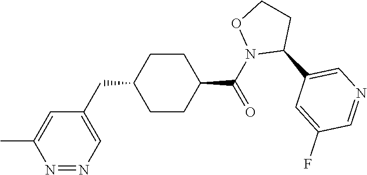 | ¹H NMR (400 MHz, MeOD-d4) δ ppm = 8.88 (d, J = 1.5 Hz, 1H), 8.21 (s, 1H), 7.52-7.40 (m, 2H), 5.49- 5.30 (m, 1H), 4.32 (td, J = 7.8, 2.9 Hz, 1H), 4.04- 3.91 (m, 1H), 3.01-2.88 (m, 1H), 2.87-2.71 (m, 1H), 2.65 (s, 3H), 2.58 (br d, J = 6.8 Hz, 2H), 2.47 (d, J = 2.4 Hz, 3H), 2.39-2.27 (m, 1H), 2.01-1.89 (m, 1H), 1.85-1.59 (m, 4H), 1.52-1.34 (m, 2H), 1.23-1.03 (m, 2H). LC/MS: m/z 399.3 [M + H]⁺, Rt (I = 220 nm): 0.423 min (LC/MS method Q), calc. m/z: 399.2. |
| 399 | 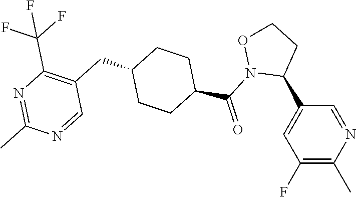 | ¹H NMR (400 MHz, DMSO-d6) δ = 8.93 (s, 1H), 8.23 (s, 1H), 7.47 (dd, J = 10., 1.75 Hz, 1H), 5.34 (dd, J = 6.4, 8.3 Hz, 1H), 4.27 (td, J = 7.8, 2.9 Hz, 1H), 3.94- 3.80 (m, 1H), 2.91-2.81 (m, 1H), 2.74-2.61 (m, 6H), 2.42 (d, J = 2.9 Hz, 3H), 2.30-2.18 (m, 1H), 1.91- 1.83 (m, 1H), 1.75-1.49 (m, 4H), 1.36-1.22 (m, 2H), 1.18-1.02 (m, 2H). LC/MS: m/z 467.3 [M + H]⁺, Rt (I = 220 nm): 0.612 min (LC/MS method Q), calc. m/z: 467.2. |
| 400 | 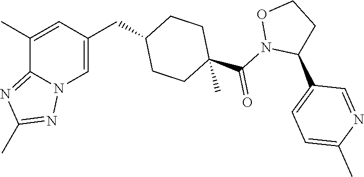 [SMILES: Cc1ccc([C@@H]2CCON2C(=O)[C@]2(C)CC[C@@H](Cc3cc(C)c4nc(C)nn4c3)CC2)cn1] | ¹H NMR (400 MHz, MeOD-d4) δ ppm = 8.33 (d, J = 2.4 Hz, 1H), 8.30 (s, 1H), 7.63 (dd, J = 7.8, 2.4 Hz, 1H), 7.33 (s, 1H), 7.27 (d, J = 7.8 Hz, 1H), 5.35 (dd, J = 8.8, 6.8 Hz, 1H), 4.33 (dt, J = 7.7, 2.2 Hz, 1H), 4.05- 3.83 (m, 1H), 3.00-2.83 (m, 1H), 2.60 (br d, J = 6.4 Hz, 2H), 2.56-2.46 (m, 9H), 2.33-2.20 (m, 1H), 1.93- 1.53 (m, 7H), 1.40-1.21 (m, 5H). LC/MS: m/z 448.3 [M + H]⁺, Rt (I = 220 nm): 0.402 min (LC/MS method Q), calc. m/z: 448.2. |
| 401 | 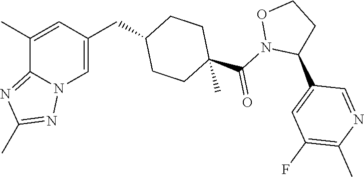 [SMILES: Cc1nc2c(C)cc(C[C@H]3CC[C@](C)(C(=O)N4OCC[C@H]4c4cnc(C)c(F)c4)CC3)cn2n1] | ¹H NMR (400 MHz, MeOD-d4) δ ppm = 8.30 (s, 1H), 8.20 (s, 1H), 7.43 (dd, J = 10.0, 1.7 Hz, 1H), 7.33 (s, 1H), 5.47-5.26 (m, 1H), 4.33 (td, J = 7.9, 2.2 Hz, 1H), 4.02-3.82 (m, 1H), 2.97-2.85 (m, 1H), 2.60 (br d, J = 6.8 Hz, 2H), 2.56-2.49 (m, 6H), 2.46 (d, J = 2.9 Hz, 3H), 2.33-2.23 (m, 1H), 1.95-1.53 (m, 7H), 1.37-1.19 (m, 5H). LC/MS: m/z 448.3 [M + H]⁺, Rt (I = 220 nm): 0.421 min (LC/MS method Q), calc. m/z: 448.2. |
| 402 | 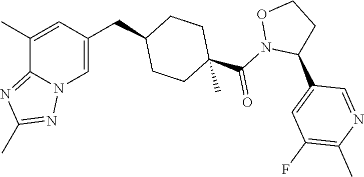 [SMILES: Cc1nc2c(C)cc(C[C@H]3CC[C@@](C)(C(=O)N4OCC[C@H]4c4cnc(C)c(F)c4)CC3)cn2n1] | ¹H NMR (400 MHz, MeOD-d4) δ ppm = 8.32-8.21 (m, 2H), 7.47 (dd, J = 10.3, 1.5 Hz, 1H), 7.29 (s, 1H), 5.48-5.39 (m, 1H), 4.34 (td, J = 7.8, 2.4 Hz, 1H), 4.08-3.90 (m, 1H), 3.01-2.86 (m, 1H), 2.62-2.43 (m, 12H), 2.40-2.22 (m, 2H), 1.68-1.50 (m, 3H), 1.28-1.02 (m, 7H). LC/MS: m/z 466.3 [M + H]⁺, Rt (I = 220 nm): 0.486 min (LC/MS method Q), calc. m/z: 466.2. |
| 403 | 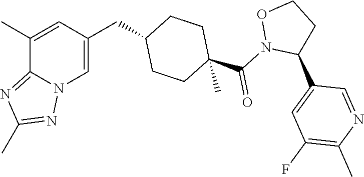 [SMILES: Cc1nc2c(C)cc(C[C@H]3CC[C@](C)(C(=O)N4OCC[C@H]4c4cnc(C)c(F)c4)CC3)cn2n1] | ¹H (400 MHz, MeOD-d4) δ ppm = 8.30 (s, 1H), 8.20 (s, 1H), 7.43 (dd, J = 10.0, 1.7 Hz, 1H), 7.33 (s, 1H), 5.47-5.26 (m, 1H), 4.33 (td, J = 7.9, 2.2 Hz, 1H), 4.02-3.82 (m, 1H), 2.97-2.85 (m, 1H), 2.60 (br d, J = 6.8 Hz, 2H), 2.56-2.49 (m, 6H), 2.46 (d, J = 2.9 Hz, 3H), 2.33-2.23 (m, 1H), 1.95-1.53 (m, 7H), 1.37- 1.19 (m, 5H) LC/MS: m/z 466.3 [M + H]+, Rt (I = 220 nm): 0.473 min (LC/MS method Q), calc. m/z: 466.2. |
| 404 | 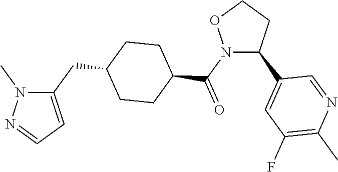 | ¹H NMR (400 MHz, MeOD-d4) δ ppm = 8.22 (s, 1H), 7.46 (dd, J = 10.3, 1.5 Hz, 1H), 7.34 (d, J = 2.0 Hz, 1H), 6.06 (d, J = 2.0 Hz, 1H), 5.42-5.33 (m, 1H), 4.32 (td, J = 7.8, 2.9 Hz, 1H), 4.05-3.89 (m, 1H), 3.77 (s, 3H), 3.00-2.88 (m, 1H), 2.87-2.70 (m, 1H), 2.58 (d, J = 7.3 Hz, 2H), 2.47 (d, J = 2.9 Hz, 3H), 2.42- 2.26 (m, 1H), 1.99-1.91 (m, 1H), 1.89-1.74 (m, 3H), 1.66-1.52 (m, 1H), 1.51-1.35 (m, 2H), 1.20- 1.02 (m, 2H). LC/MS: m/z 387.3 [M + H]⁺, Rt (I = 220 nm): 0.529 min (LC/MS method Q), calc. m/z: 387.2. |
| 405 | 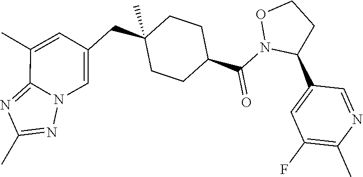 [SMILES: Cc1nc2c(C)cc(C[C@]3(C)CC[C@H](C(=O)N4OCC[C@H]4c4cnc(C)c(F)c4)CC3)cn2n1] | ¹H NMR (400 MHz, MeOD-d4) δ ppm = 8.31-8.24 (m, 2H), 7.51 (dd, J = 9.9, 1.2 Hz, 1H), 7.28 (s, 1H), 5.49-5.40 (m, 1H), 4.35 (td, J = 7.7, 3.1 Hz, 1H), 4.09-3.95 (m, 1H), 3.03-2.92 (m, 1H), 2.91-2.83 (m, 1H), 2.72 (s, 2H), 2.59-2.46 (m, 9H), 2.41-2.30 (m, 1H), 1.93-1.78 (m, 3H), 1.73-1.56 (m, 3H), 1.39- 1.22 (m, 2H), 0.87 (s, 3H). LC/MS: m/z 466.2 [M + H]⁺, Rt (I = 220 nm): 0.467 min (LC/MS method Q), calc. m/z: 466.2. |
| 406 | 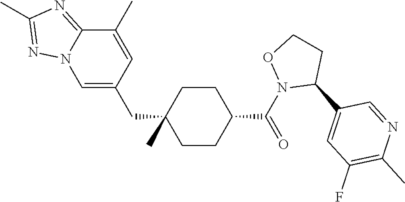 | ¹H (400 MHz, MeOD-d4) δ ppm = 8.34-8.28 (m, 1H), 8.26-8.21 (m, 1H), 7.48 (dd, J = 10.1, 1.3 Hz, 1H), 7.32 (s, 1H), 5.46-5.36 (m, 1H), 4.33 (td, J = 7.8, 2.9 Hz, 1H), 4.04-3.90 (m, 1H), 3.02-2.90 (m, 1H), 2.82- 2.70 (m, 1H), 2.60 (s, 2H), 2.58-2.54 (m, 6H), 2.49 (d, J = 2.8 Hz, 3H), 2.41-2.29 (m, 1H), 1.86-1.75 (m, 1H), 1.72-1.57 (m, 3H), 1.56-1.36 (m, 4H), 0.97 (s, 3H) LC/MS: m/z 466.2 [M + H]⁺, Rt (I = 220 nm): 0.465 min (LC/MS method Q), calc. m/z: 466.2. |
| 407 | 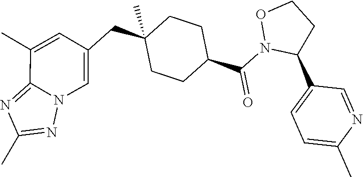 [SMILES: Cc1ccc([C@@H]2CCON2C(=O)[C@H]2CC[C@@](C)(Cc3cc(C)c4nc(C)nn4c3)CC2)cn1] | 1H (400 MHz, MeOD-d4) δ ppm = 8.39 (d, J = 2.0 Hz, 1H), 8.27 (s, 1H), 7.70 (dd, J = 8.1, 2.2 Hz, 1H), 7.31 (d, J = 8.0 Hz, 1H), 7.27 (s, 1H), 5.45-5.36 (m, 1H), 4.34 (td, J = 7.8, 3.1 Hz, 1H), 4.06-3.95 (m, 1H), 3.02- 2.91 (m, 1H), 2.91-2.81 (m, 1H), 2.72 (s, 2H), 2.57- 2.49 (m, 9H), 2.41-2.28 (m, 1H), 1.92-1.78 (m, 3H), 1.72-1.54 (m, 3H), 1.38-1.21 (m, 2H), 0.87 (s, 3H) LC/MS: m/z 448.3 [M + H]⁺, Rt (I = 220 nm): 0.411 min (LC/MS method Q), calc. m/z: 448.2. |
| 408 | 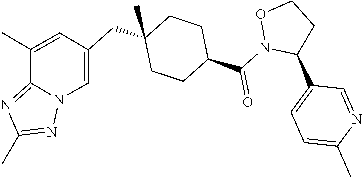 [SMILES: Cc1ccc([C@@H]2CCON2C(=O)[C@H]2CC[C@](C)(Cc3cc(C)c4nc(C)nn4c3)CC2)cn1] | 1H (400 MHz, MeOD-d4) δ ppm = 8.43 (d, J = 2.1 Hz, 1H), 8.30 (s, 1H), 7.89 (dd, J = 8.1, 1.9 Hz, 1H), 7.48 (d, J = 8.1 Hz, 1H), 7.32 (s, 1H), 5.44-5.37 (m, 1H), 4.33 (td, J = 7.8, 2.8 Hz, 1H), 4.03-3.91 (m, 1H), 3.01-2.91 (m, 1H), 2.79-2.67 (m, 1H), 2.62-2.58 (m, 5H), 2.56-2.52 (m, 6H), 2.39-2.28 (m, 1H), 1.83- 1.73 (m, 1H), 1.71-1.55 (m, 3H), 1.53-1.36 (m, 4H), 0.95 (s, 3H) LC/MS: m/z 448.3 [M + H]⁺, Rt (I = 220 nm): 0.410 min (LC/MS method Q), calc. m/z: 448.2. |
| 409 | 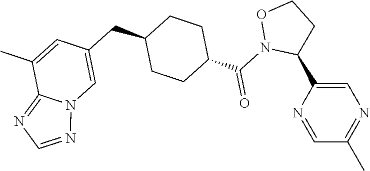 | 1H NMR (400 MHz, CDCl3) δ = 8.52 (d, J = 1.6 Hz, 1H), 8.39 (s, 1H), 8.27 (s, 1H), 8.21 (s, 1H), 7.14 (s, 1H), 5.48 (dd, J = 8.8, 6.0 Hz, 1H), 4.39-4.25 (m, 1H), 4.02-3.85 (m, 1H), 2.83-2.67 (m, 3H), 2.64 (s, 3H), 2.59-2.48 (m, 5H), 2.01-1.92 (m, 1H), 1.81 (d, J = 11.6 Hz, 3H), 1.66-1.56 (m, 1H), 1.55-1.39 (m, 2H), 1.18-0.99 (m, 2H). LC/MS: m/z 421.3 [M + H]⁺, Rt (I = 220 nm): 0.545 min (LC/MS method Q), calc. m/z: 421.3. |
| 410 | 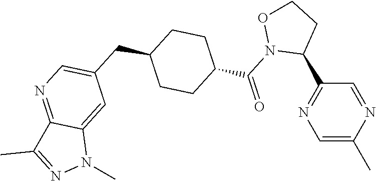 | ¹H NMR (400 MHz, CDCl₃) δ = 8.52 (d, J = 1.2 Hz, 1 H), 8.39 (s, 1 H), 8.36 (d, J = 1.2 Hz, 1 H), 7.46 (s, 1 H), 5.48 (dd, J = 8.8, 5.6 Hz, 1 H), 4.31 (td, J = 8.8, 5.6 Hz, 1 H), 4.01 (s, 3 H), 3.98-3.90 (m, 1 H), 2.83- 2.75 (m, 2 H), 2.73-2.67 (m, 3 H), 2.66 (s, 3 H), 2.55 (s, 3 H), 1.96 (d, J = 12.0 Hz, 1 H), 1.87-1.78 (m, 3 H), 1.69-1.59 (m, 1 H), 1.55-1.38 (m, 2 H), 1.20- 1.03 (m, 2 H). LC/MS: m/z 435.3 [M + H]⁺, Rt (I = 220 nm): 0.808 min (LC/MS method Q), calc. m/z: 435.2. |
| 411 | 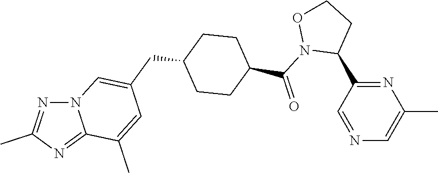 | ¹H (400 MHz, DMSO-d6) δ = 8.44 (s, 2H), 8.34 (s, 1H), 7.28 (s, 1H), 5.39-5.28 (m, 1H), 4.35-4.20 (m, 1H), 3.98-3.85 (m, 1H), 2.85-2.74 (m, 1H), 2.72- 2.61 (m, 1H), 2.52-2.48 (m, 2H, below solvent signal), 2.48-2.46 (m, 7H), 2.43 (s, 3H), 1.93-1.82 (m, 1H), 1.76-1.65 (m, 3H), 1.61-1.48 (m, 1H), 1.38-1.25 (m, 2H), 1.11-0.94 (m, 2H) LC/MS: m/z 435.2 [M + H]⁺, Rt (I = 220 nm): 0.1.170 min (LC/MS method A), calc. m/z: 435.2. |

### Process of Making

The compounds of formula (I) may be prepared using the methods disclosed herein and routine modifications thereof, which will be apparent given the disclosure herein and methods well known in the art.

### Scheme 1: General Synthesis of Compounds of Formula (I), Wherein R1, R2, R4, R5, m, s, p, q, r and t are as Defined Here-Above

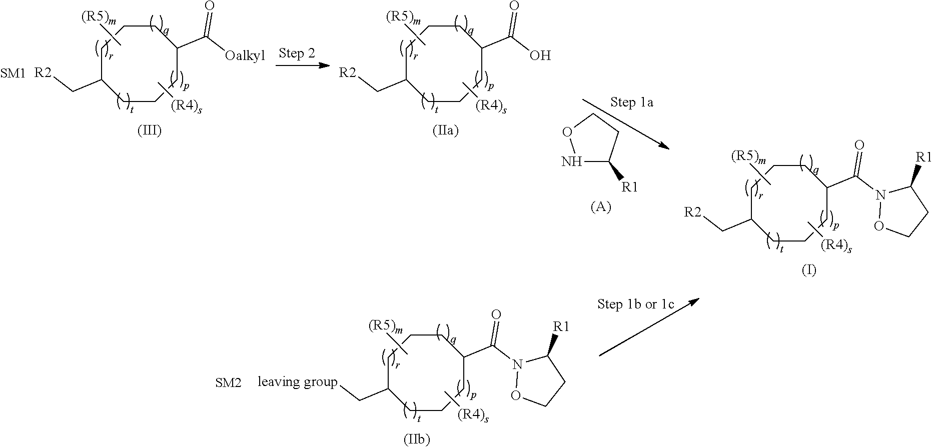

According to a first Synthetic Method (SM1), compounds of formula (I) can be obtained from an alkyl ester of formula (III). Step 2 consists in hydrolysis of the alkyl ester of formula (III) to form the compound of formula (IIa) and can be achieved under well-known conditions using e.g. lithium hydroxide or sodium hydroxide in water or a solvent mixture such as THF/water or THF/water/MeOH. Step 1a is an Amide coupling between an appropriate substituted isoxazolidine compound of formula (A) and the compound of formula (IIa). It can be achieved by standard acid activation methods using for example ethyl cyanohydroxyiminoacetate (Oxyma), acid chloride, HOBt, HATU, HBTU, PyBOP or 1-propanephosphonic anhydride (T₃P) under basic conditions, e.g. diisopropylethylamine or the like, in aprotic solvents like DMF, DMSO, acetonitrile, or the like to form compounds of formula (I).

Substituted isoxazolidines of formula (A) can be synthesized by known procedures from the literature, e.g. WO2017096301, WO2019130230 and WO2021245070.

According to a second Synthetic Method (SM2), compounds of formula (I) can be obtained from an isoxazolidine derivative of formula (IIb) where the leaving group can represent a fluorine, chlorine, iodine or bromine atom, a sulfonate group, O-nonaflate or a O-triflate.

In the case where R2 represents an optionally substituted heteroaryl group containing a Nitrogen atom compound of formula (I) can be obtained from the isoxazolidine derivative of formula (IIb) via alkylation Step 1b. Compound (IIb) is reacted with an appropriate heteroaromatic compound containing a Nitrogen atom in inert solvents, as for example DMF or THF in the presence of bases such as NaH, Cs₂CO₃ or K₂CO₃.

In the case where R2 represents an aryl or heteroaryl group not containing a Nitrogen atom, compound of formula (I) can be obtained from the isoxazolidine derivative of formula (IIb) via Step 1c using for example photoredox catalysis (described in detail in JACS 2016, 138, 8084-8087) or cross electrophile coupling (described in detail in ACS Catal 2020, 10, 12642-12656).

Compounds of formula (IIb) can be synthesized by known procedures from the literature or as described herein.

### Scheme 2: Synthesis of Compounds of Formula (III) Wherein R2 is an Optionally Substituted Benzimidazolyl Group.

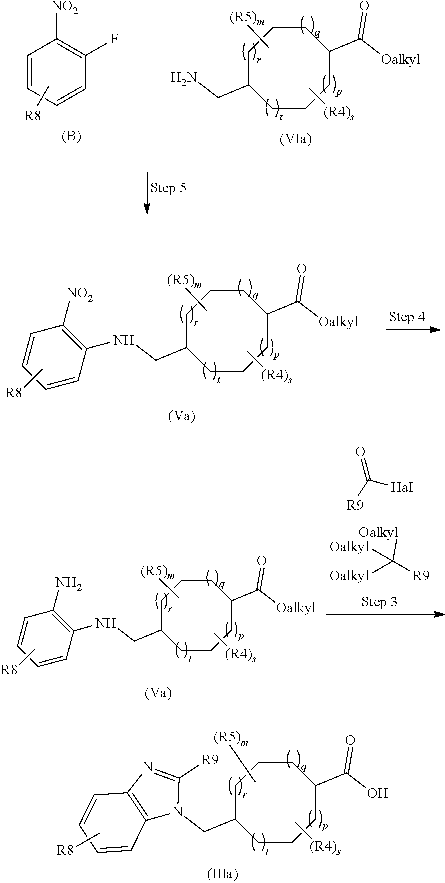

Compounds of formula (I) with R2 representing an optionally substituted imidazolyl group can be synthesized starting from optionally substituted ortho fluoro nitrobenzene (B) and a compound of formula (IVa). In Scheme 2, R4, R5, R6, m, s, p, q, r and t are as defined above for formula (I), o is 0, 1, 2 or 3, R8 either represents a hydrogen atom or a R6 group as defined above R9 represents a hydrogen atom or a group selected from halogen, cyano, —OH, a (C₁-C₄)alkyl group, CF₃, —C(O)NH₂, a C(O)NH(C₁C₄)-alkyl group, —C(O)OH, a —C(O)O—(C₁-C₄)alkyl group, SO₂NH₂ and a (C₁-C₄)alkoxy group.

Step 5 is a S_{N}Ar reaction implemented in the presence of a base, such as diisopropylethylamine, in an appropriate solvent, such as acetonitrile. The obtained nitro aniline compound (Va) can be subsequently transformed (Step 4) into the corresponding amino aniline compound (IVa), which can be achieved by e.g. palladium catalyzed reduction under hydrogen atmosphere in an appropriate solvent, such as methanol. Step 3 allows obtaining compound (Illa) via condensation with appropriate ortho esters or acid halides bearing a R8 radical, for example acetyl chloride or trimethyl orthoformate. This type of reactions can be run in solvents such as methanol or dioxane, respectively.

Optionally substituted ortho fluoro nitrobenzene (B) can be synthesized by known procedures from the literature.

Compounds of formula (VIa) can be synthesized by known procedures from the literature or as described herein.

Scheme 3: Synthesis of Compounds of Formula (III), with R2 Representing an Optionally Substituted Aryl or Heteroaryl Group, Different from a Benzimidazolyl.

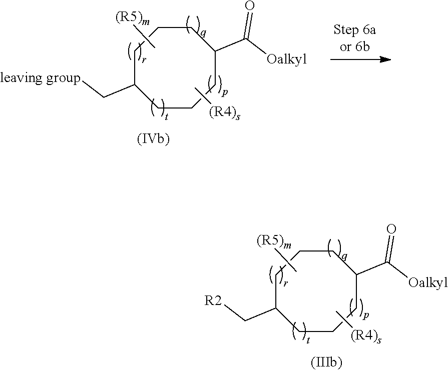

Compounds of the general formula (IIIb) where R2 represents an optionally substituted heteroaryl group containing a Nitrogen atom compound can be synthesized starting from compound (IVb) via alkylation (step 6a) within the same conditions detailed here-above for step 1b in SM2. Leaving groups can be an iodine or bromine atom, or a sulfonate group.

In the case where R2 represents an aryl or heteroaryl group not containing a Nitrogen atom, compound of formula (IIIb) can be obtained from compound (IVb) via step 6b by photoredox catalysis or cross electrophile coupling similarly to Step Ic of general procedure SM2.

Compounds of formula (IVb) can be synthesized by known procedures from the literature (like March's Advanced Organic Chemistry, John Wiley & Sons; 2019) or as described herein.

Functional groups like acids, esters, amides, nitriles, halogens in compounds can be transformed (functional group interconversion) into other functional groups with standard methods like esterification, saponification, halogenation, Suzuki reaction to yield further compounds of formula (I).

The synthesis of typical compounds described herein may be accomplished as described in the following examples. If available, reagents may be purchased commercially, e.g., from Sigma Aldrich or other chemical suppliers. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedure.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006), Greene's protective groups in organic synthesis, Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989). The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane, “DCM”), diethyl ether, methanol, pyridine and the like). Unless specified to the contrary, the solvents used in the reactions of the present disclosure are inert organic solvents, and the reactions are carried out under an inert gas, preferably argon.

### Pharmaceutical Compositions

The compounds provided herein is usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients.

According to another aspect, pharmaceutical compositions are disclosed that include a compound described herein as an active ingredient. The compounds of the disclosure will normally, but not necessarily, be formulated into pharmaceutical compositions prior to administration to a patient These pharmaceutical compositions comprise an effective dose of at least one compound of the disclosure as defined herein, or a pharmaceutically acceptable salt thereof, and one or more pharmaceutically acceptable excipients.

The said excipients are selected, in accordance with the pharmaceutical form and method of administration desired, from the customary excipients, which are known to a person skilled in the art.

In these pharmaceutical compositions for oral, sublingual, subcutaneous, intramuscular, intravenous, topical, local, intra-tracheal, intranasal, transdermal or rectal administration, the active ingredient is a compound of formula (I) above, or its salt or solvate where appropriate, may be administered in a unit administration form, in a mixture with conventional pharmaceutical excipients, to a subject like human beings for the prophylaxis or treatment of a disease or disorder or condition that is at least partly mediated by receptor-interacting protein kinase 1 and in particular acute and chronic neurodegenerative diseases like Parkinson's disease, Alzheimer's disease, amyotrophic lateral sclerosis (ALS) or multiple sclerosis (MS).

### Unit Administration

The unit administration forms appropriate include oral forms such as tablets, soft or hard gel capsules, powders, granules and oral solutions or suspensions, sublingual, buccal, intratracheal, intra-ocular and intranasal administration forms, forms for inhalative, topical, transdermal, sub-cutaneous, intra-muscular or intravenous administration, rectal administration forms and implants.

When prepared in unit administration form, the pharmaceutical compositions of the disclosure typically contain from 1 mg to 1000 mg of the active ingredient. The amount of active ingredient that is combined with one or more excipients to produce a single unit administration form will necessarily vary depending upon the host treated and the particular route of administration. For example, a formulation intended for oral administration to humans will generally contain, for example, from 0.5 mg to 0.5 g of active agent compounded with an appropriate and convenient amount of excipients which may vary from about 5 to about 98 percent by weight of the total composition.

As an example, a unit administration form of a herein described compound in tablet form may comprise the following components:

| | | |
|---|---|---|
| Compound | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

In using a compound of the disclosure for therapeutic or prophylactic purposes it will generally be administered so that a daily dose in the range, for example, from 0.1 mg/kg to 75 mg/kg body weight is received, given if required in divided doses.

In general, lower doses will be administered when a parenteral route is employed. Thus, for example, for intravenous or intraperitoneal administration, a dose in the range, for example, from 0.1 mg/kg to 30 mg/kg body weight will generally be used. Oral administration may also be suitable, particularly in tablet form. Typically, unit dosage forms will contain about from 0.5 mg to 0.5 g of a compound of this disclosure.

There may be particular cases in which higher or lower dosages are appropriate; such dosages do not depart from the scope of the disclosure. According to usual practice, the dosage that is appropriate for each patient is determined by the doctor according to the mode of administration and the weight and response of the said patient.

### Methods of Treatment

In other embodiments, provided herein is a method of treating a receptor-interacting protein kinase 1-mediated disease or disorder. The method includes administering a therapeutically effective amount of a compound or pharmaceutical composition as described herein to a subject in need thereof. In some embodiments, the receptor-interacting protein kinase 1-mediated disease or disorder is Parkinson's disease, Alzheimer's disease, amyotrophic lateral sclerosis (ALS) or multiple sclerosis (MS).

The receptor-interacting protein kinase 1 inhibitors of the present disclosure are therefore useful for treating diseases and conditions mediated by receptor-interacting protein kinase 1, including but not limited to neurodegenerative diseases, central nervous system (CNS) diseases.

### Neurodegenerative and CNS Diseases

The receptor-interacting protein kinase 1 inhibitors described herein may also be used to treat neurodegenerative diseases. Neurodegenerative diseases can affect many of the body's activities, such as balance, movement, talking, breathing, and heart function. Neurodegenerative diseases can be genetic or caused by medical conditions such as alcoholism, tumors, strokes, toxins, chemicals, and viruses. Non-limiting examples of neurodegenerative diseases include Alzheimer's disease, amyotrophic lateral sclerosis (ALS) and Parkinson's disease.

In certain embodiments, the compounds and compositions of the present disclosure are useful for treating Alzheimer's disease. In certain embodiments, the compounds and compositions of the present disclosure are useful for treating Parkinson's disease. In certain embodiments, the compounds and compositions of the present disclosure are useful for treating amyotrophic lateral sclerosis (ALS).

More generally, the receptor-interacting protein kinase 1 inhibitors described herein can be used to preserve neuron viability and promote axon growth and nerve functions within the central nervous system (CNS). Accordingly, the compounds may be used to reduce or even reverse the loss of cognitive, motor, and sensory functions associated with a CNS disease or disorder, by preserving neuron viability and/or promoting axon regeneration and/or nerve functions.

If desired, the effective daily dose of the active compound may be administered as two, three, four, five, six or more sub-doses administered separately at appropriate intervals throughout the day, optionally, in unit dosage forms. In certain embodiments, the present disclosure relates to compounds for inhibiting cell death, wherein the compounds are represented by formula (I). In certain embodiments, the compounds of the present disclosure are inhibitors of cell death. In any event, the compounds of the present disclosure in another embodiment exert their effect on inhibiting cell death at a concentration less than about 50 micromolar, more in another embodiment at a concentration less than about 10 micromolar and most in another embodiment at a concentration less than 1 micromolar. The compounds of the disclosure can be tested in standard animal models of stroke and standard protocols such as described by Hara, H., et al. Proc. Natl. Acad. Sci. USA, 1997. 94(5): 2007-12.

When the compounds of the present disclosure are administered as pharmaceuticals, to humans and animals, they can be given per se or as a pharmaceutical composition containing, for example, 0.1% to 99.5% (more in another embodiment, 0.5% to 90%) of active ingredient in combination with a pharmaceutically acceptable carrier.

The compounds of the present disclosure or the compositions thereof may be administered once, twice, three or four times daily, using any suitable mode described above. Also, administration or treatment with the compounds may be continued for a number of days; for example, commonly treatment would continue for at least 7 days, 14 days or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be between 1 mg and 1,000 mg, between about 1,000-2,000 mg/day, between about 10-500 mg/day, between about 50-300 mg/day, between about 75-200 mg/day or between about 100-150 mg/day.

The daily dosage may also be described as a total amount of a compound described herein administered per dose or per day. Daily dosage of a compound may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day or between about 15 to 150 mg/day. In certain embodiments, the method comprises administering to the subject an initial daily dose of about 1 to 800 mg of a compound described herein and increasing the dose by increments until clinical efficacy is achieved. Increments of about 5, 10, 25, 50 or 100 mg can be used to increase the dose. The dosage can be increased daily, every other day, twice per week or once per week.

In certain embodiments, a compound or pharmaceutical preparation is administered orally. In certain embodiments, the compound or pharmaceutical preparation is administered intravenously. Alternative routes of administration include sublingual intramuscular and transdermal administrations.

The preparations of the present disclosure may be given orally, parenterally, topically or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the administration is oral.

### EXAMPLES

The following working examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. Thus, while the present disclosure has been described above with particularity, the following examples provide further detail in connection with what are presently deemed to be the most practical embodiments of the disclosure.

### Abbreviations

| | |
|---|---|
| ACN | Acetonitrile |
| ADP | Adenosindiphosphate |
| ATP | Adenosintriphosphate |
| BOC | Tertiar butyl-oxy-carbonyl |
| BPD | 4,4,4′,4′,5,5,5′,5′-octamethyl-2,2′-bi-1,3,2- |
| | dioxaborolane |
| br | Broad |
| CDI | Carbonyldiimidazole |
| CMC | Carboxy-methyl-cellulose |
| CRC | Concentration response curve |
| d | Dublett |
| DBU | 1,8-Diazabicyclo[5.4.0]undec-7-en |
| DCM | Dichloromethane |
| dd | Dublett of dubletts |
| DEA | Diethylamine |
| DIAD | Diisopropyl azodicarboxylate |
| DMAP | Dimethylaminopyridine |
| DME | Dimethoxyethane |
| DMF | Dimethylformamide |
| DMSO | Dimethylsulfoxide |
| DPPF | 1,1′-Bis(diphenylphosphino)ferrocen |
| dt | Dublett of tripletts |
| dtbbpy | 4,4′-di-tert-butyl-2,2′-bipyridine |
| EA | Ethyl acetate |
| eq. | Equivalent |
| ES | Electro spray |
| EtOH | Ethanol |
| FBS | Fetal bovine serum |
| HATU | 1-[Bis(dimethylamino)methylene]-1H- |
| | 1,2,3-triazolo[4,5-b]pyridinium |
| | 3-oxide hexafluorophosphate |
| HBTU | 2-(1H-Benzotriazole-1-yl)-1,1,3,3- |
| | tetramethyluronium hexafluorophosphate |
| HOBt | 1-Hydroxybenzotriazole |
| HPLC | High pressure liquid chromatography |
| hr/hrs | Hour/hours |
| Hz | Hertz |
| IL6 | Interleukin 6 |
| IPA | Isopropylalcohol |
| Ir[dF(CF₃)ppy]₂(dtbpy)(PF₆) | [4,4′-Bis(1,1-dimethylethyl)-2,2′- |
| | bipyridine-N1,N1′]bis[3,5-difluoro-2-[5- |
| | (trifluoromethyl)-2-pyridinyl-N]phenyl-C] |
| | Iridium(III) hexafluorophosphate, |
| | [Ir{dF(CF₃)ppy}₂(dtbpy)]PF₆ |
| [Ir(dF(Me)ppy)₂(dtbbpy)]PF₆ | [(4,4′-Di-tert-butyl-2,2′-bipyridine)-bis-(5- |
| | methyl-2-(5-fluoro-phenyl)-pyridine)- |
| | iridium(III)] hexafluorophosphate |
| LC/MS | Liquid chromatography mass spectrum |
| m | Multiplett |
| m/z | Mass per charge |
| MeOH | Methanol |
| MHz | Mega Hertz |
| min | Minute(s) |
| MTBE | 2-Methoxy-2-methylpropan |
| N | Normal |
| n-BuOH | n-Butanol |
| NBS | N-bromosuccinimide |
| NMR | Nuclear magnetic resonance |
| Oxyma | Ethyl cyanohydroxyiminoacetate |
| PBS | Phosphate buffered saline |
| PG | Protecting group |
| PyBOP | Benzotriazol-1-yloxytripyrrolidino- |
| | phosphonium hexafluorophosphate |
| q | Quadruplett |
| quin | Quintuplett |
| rH | Relative humidity |
| rt | Room temperature |
| Rt | Retention time |
| s | Singulett |
| S-PHOS | 2-Dicyclohexylphosphino-2′,6′- |
| | dimethoxybiphenyl |
| SFC | Supercritical fluid chromatography |
| SNAr | Nucleophilic Aromatic Substitution |
| t | Triplett |
| T₃P | Propanephosphonic acid anhydride |
| TBAF | Tetra-n-butylammonium fluoride |
| td | Triplett of dubletts |
| TDAE | Tetrakis(dimethylamino)ethylene |
| TEA | Triethyl amine |
| TFA | Trifluoroacetic acid |
| TFAA | Trifluoroacetic acid anhydride |
| THF | Tetrahydrofurane |
| TLC | Thin layer chromatography |
| TNF | Tumor Necrosis Factor |
| TTMSS | tris(trimethylsilyl)silane |
| UV | Ultraviolet |
| X-PHOS | 2-Dicyclohexylphosphin-2′,4′,6′- |
| | triisopropylbiphenyl |

### Silica Gel Chromatography

Silica gel chromatography was performed using CombiFlash® Rf (Teledyne ISCO), Biotage Isolera One automated flash purification system or two Büchi systems (C-660, C-605, C-620, C-635 combination and C-660, C-605, C-615, C-630 combination) with pre-packed cartridges.

### Preparative Reversed-Phase HPLC

For preparative reversed-phase HPLC an Agilent 1200 preparative HPLC machine, Gilson equipment (GX-271 liquid handler, 331/332-pump, UV/VIS-155) or a Waters Autopurification LC Prep System was used.

### Preparative RP-LC

Reversed phase liquid chromatography was performed with a Biotage equipment using C18 columns and a water (0.1% formic acid)/acetonitrile gradient.

### NMR

400 MHz: ¹H NMR spectra were recorded on a Bruker AVANCE II 400 spectrometer operating at a proton frequency of 400.23 MHz. The instrument was equipped with a 5 mm BBI room temperature probe head. Alternatively, a Bruker AVANCE III HD 400 MHz, or a Bruker AVANCE NEO 400 MHz was used.

600 MHz: ¹H NMR spectra were recorded on a Bruker AVANCE III 600 spectrometer operating at a proton frequency of 600.05 MHz. The instrument was equipped with a 5 mm BBI room temperature probe head.

### Analytical LC/MS Equipment for Method A

Retention time and mass detection were done on a Waters Acquity UHPLC system coupled with a Waters SQD mass detector. The injection volume was 1.0 μl. Molecular weights are given in gram per mol [g/mol], detected masses in mass per charge [m/z].

### Analytical LC/MS Equipment for Methods B, D and E

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### Analytical LC/MS Equipment for Methods C, Q, R, S

Retention time and mass detection were done on a SHIMADZU LC-30AD system coupled with a PDA or DAD mass detector

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### Chiral Analytical Equipment for Method F, I, M, Y, AE, AF, AG, AR and AS

For retention time a SFC: SHIMADZU LC-30AD sf system was used.

### Analytical LC/MS Equipment for Method P, W, X, Z, AC and AH

For retention time a LC system from Agilent (Agilent-1260 series system) was used

### Analytical LC/MS Equipment for Method A

Retention time and mass detection were done on a Waters Acquity UHPLC system coupled with a Waters SQD mass detector. The injection volume was 1.0 μl. Molecular weights are given in gram per mol [g/mol], detected masses in mass per charge [m/z].

### Analytical LC/MS Equipment for Method B and Method C

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### LC/MS-Method A

Gradient: 98% H₂O (0.05% formic acid)/2% acetonitrile (0.035% formic acid) for 0.2 min, then from 98% H₂O (0.05% formic acid) to 98% acetonitrile (0.035% formic acid) in 3.6 min, then 98% acetonitrile (0.035% formic acid) for 0.5 min, flow rate: 1.0 ml/min, column: 2.1×50 mm Waters ACQUITY UPLC BEH C18, 1.7 μm, 55° C. UV data: retention time ad λ=220 nm given in min MS data: ES+ ionisation, m/z given as [M+H]+ unless otherwise noted.

### LC/MS-Method B

Gradient: From 95% H₂O (0.0375% TFA)/5% acetonitrile (0.01875% TFA) to 5% H₂O (0.0375% TFA)/95% acetonitrile (0.01875% TFA) in 0.8 min, flow rate: 1.5 ml/min, column: Kinetex EVO C18 2.1×30 mm, 5 μm, 50° C. UV data: retention time ad λ=220 nm given in min MS data: ES+ ionisation, m/z given as [M+H]+ unless otherwise noted.

### LC/MS-Method C

Gradient: From 100% H2O (0.0375% TFA)/0% acetonitrile (0.01875% TFA) to 60% H2O (0.0375% TFA)/40% acetonitrile (0.01875% TFA) in 0.8 min, flow rate: 1.5 ml/min, column: Kinetex EVO C18 2.1×30 mm, 5 μm, 50° C. UV data: retention time ad λ=220 nm given in min MS data: ES+ ionisation, m/z given as [M+H]+ unless otherwise noted.

### LC/MS Method Q

Gradient: From 95% H₂O (0.0375% TFA)/5% ACN (0.01875% TFA) to 5% H₂O (0.0375% TFA)/95% ACN (0.01875% TFA) in 1.05 min; flow rate: 2 ml/min, column: HALO C¹⁸ 3.0×30 mm, 5.0 um 50° C. UV data: retention time ad λ 220 nm given in min. Detector: PDA

MS data: ES+ ionisation, m/z given as [M+H]+ unless otherwise noted

### LC/MS Method R

Gradient: From 95% H2O (0.025% NH3·H2O)/5% ACN to 5% H2O (0.025% NH3·H2O)/5% ACN in 1.05 min; Maintain 5% A:95% B for 0.4 min, then back to 95% A:5% B in 0.3 min flow rate: 2 ml/min, column: Kinetex EVO C18 2.1×30 mm, 5 μm, 50° C. UV data: retention time ad λ 220 nm given in min. Detector PDA

MS data: ES+ ionisation, m/z given as [M+H]+ unless otherwise noted

### LC/MS Method S

Gradient: From 95% H2O (0.025% NH3·H2O)/5% ACN to 5% H2O (0.025% NH3·H2O)/5% ACN in 1.05 min; Maintain 5% A:95% B for 0.4 min, then back to 95% A:5% B in 0.3 min flow rate: 2 ml/min, column: Kinetex EVO C18 2.1×30 mm, 5 μm, 40° C. UV data: retention time ad λ 220 nm given in min. Detector PDA

MS data: ES+ ionisation, m/z given as [M+H]+ unless otherwise noted

### Chiral Analytical Methods:

### Method F

Mobile phase: Phase A for CO2, and Phase B for MeOH (0.05% DEA); Gradient: MeOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector PDA

UV data: retention time ad λ 220 nm given in min

### Method I

Mobile phase: Phase A for CO2, and Phase B for MeOH (0.05% DEA); Gradient: MeOH (0.05% DEA) in CO2 from 5% to 40%; flow rate: 3 mL/min, column: Chiralpak AD-3 50×4.6 mm I.D., 3 μm, 35° C.; Back pressure: 100 Bar; Detector PDA

UV data: retention time ad λ 220 nm given in min

### Method M

Mobile phase: Phase A for CO2, and Phase B for EtOH (0.05% DEA); Gradient: EtOH (0.05% DEA) in CO2 from 5% to 40%; flow rate: 3 mL/min, column: Chiralpak AD-3 50×4.6 mm I.D, 3 μm, 35° C.; Back pressure: 100 Bar; Detector PDA

UV data: retention time ad λ 220 nm given in min

### Method P

Mobile phase: Phase A for CO2, and Phase B for MeOH (0.05% DEA); Gradient: EtOH (0.05% DEA) in CO2 from 5% to 40%; flow rate: 3 mL/min, column: Chiralcel OD-3 50×4.6 mm, 3 μm, 35° C.; Back pressure: 100 Bar; Detector: DAD

UV data: retention time ad λ 220 nm given in min

### Method W

Mobile phase: Phase A for CO2, and Phase B for MeOH (0.05% DEA); Gradient: MeOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector PDA

UV data: retention time ad λ 220 nm given in min

### Method X

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OJ-3 50×4.6 mm I.D., 3 μm, 35° C.; Back pressure: 100 Bar; Detector PDA

UV data: retention time ad λ 220 nm given in min

### Method Z

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OD-3 50×4.6 mm, 3 μm, 35° C.; Back pressure: 100 Bar; Detector PDA

UV data: retention time ad λ 220 nm given in min

### Method AC

Mobile phase: Phase A for CO2, and Phase B for iPrOH (0.05% DEA); Gradient: iPrOH (0.05% DEA) in CO2 from 5% to 40%; flow rate: 3 mL/min, column: Chiralpak AD-3 50×4.6 mm I.D., 3 μm, 35° C.; Back pressure: 100 Bar; Detector: PDA

UV data: retention time ad λ 220 nm given in min

### Method AE

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OJ-3 50×4.6 mm, 3 μm, 35° C.; Back pressure: 100 Bar; Detector PDA

UV data: retention time ad λ 220 nm given in min

### Method AF

Mobile phase: Phase A for CO2, and Phase B for iPrOH+ACN (0.05% DEA); Gradient: iPrOH+ACN (0.05% DEA); (0.05% DEA) in CO2 from 5% to 40%; flow rate: 3 mL/min, column: Kromasil (S,S)Whelk-O1 50×4.6 mm I.D., 3 μm, 35° C.; Back pressure: 100 Bar; Detector: PDA

UV data: retention time ad λ 220 nm given in min

### Method AG

Mobile phase: Phase A for CO2, and Phase B for EtOH (0.05% DEA); Gradient: EtOH (0.05% DEA) in CO2 from 5% to 40%; flow rate: 3 mL/min, column: Chiralpak AS-3 50×4.6 mm I.D., 3 μm, 35° C.; Back pressure: 100 Bar; Detector PDA

UV data: retention time ad λ 220 nm given in min

### Method AH

Mobile phase: Phase A for CO2, and Phase B for EtOH (0.05% DEA); Gradient: EtOH (0.05% DEA) in CO2 from 5% to 40%; flow rate: 3 mL/min, column: Chiralpak AD-3 50×4.6 mm I.D., 3 μm, 35° C.; Back pressure: 100 Bar; Detector: PDA

UV data: retention time ad λ 220 nm given in min

### Method AL

Mobile phase: Phase A for CO2, and Phase B for iPrOH+ACN (0.05% DEA); Gradient: iPrOH+ACN (0.05% DEA) in CO2 from 5% to 40%; flow rate: 3 mL/min, column: Kromasil (S,S)Whelk-O1 50×4.6 mm I.D., 3 um, 35° C.; Back pressure: 100 Bar; Detector: PDA

UV data: retention time ad λ 220 nm given in min

### Method AR

Mobile phase: Phase A for CO2, and Phase B for EtOH (0.05% DEA); Gradient: EtOH (0.05% DEA) in CO2 from 5% to 40%; flow rate: 3 mL/min, column: Chiralcel OD-3 50×4.6 mm I.D., 3 μm, 35° C.; Back pressure: 100 Bar; Detector DAD

UV data: retention time ad λ 220 nm given in min

### Method AS

Mobile phase: Phase A for CO2, and Phase B for MeOH (0.05% DEA); Gradient: MeOH (0.05% DEA) in CO2 from 5% to 40%; flow rate: 3 mL/min, column: Chiralpak IC-3 50×4.6 mm I.D., 3 μm, 3 μm, 35° C.; Back pressure: 100 Bar; Detector: DAD

UV data: retention time ad λ 220 nm given in min

### Salts

In compounds described as HCl-, TFA- or as another salt the exact amount of the respective salt is usually not determined. Therefore, the amount of the salt can range from as low as 0.01 eq. up to 5.0 eq. depending on the chemical structure (e.g. number of basic centres).

### Chiral Purity

Compounds are drawn and named as a single enantiomer, if the enantiomeric ratio exceeded 90:10. For enantiomeric ratios below 90:10 the racemic form is used.

### Example 1: Synthesis of Compounds (5), (6), (7), (8), (9), (10), (11), (12), (13), (36), (37), (38), (39), (40), (48), (49), (50), (51), (64), (65), (92), (97), (98), (106), (107), (112), (113), (114), (115) and (116)

### Example 1.1: Synthesis of Compounds (7), (8), (9)

### Step 1: Synthesis of Methyl trans-4-[(2-nitroanilino)methyl]cyclohexanecarboxylate—Compound Responding to Formula (Va) as Defined in Scheme 2

To a stirred suspension of 1-fluoro-2-nitrobenzene (749 μl, 7.09 mmol) and methyl 4-(aminomethyl)cyclohexanecarboxylate hydrochloride (1.50 g, 7.09 mmol) in CH₃CN (22 ml) NEtiPr₂ was added (3.71 ml, 21.26 mmol) at rt. The solution was heated at reflux for 2 hrs. Volatile components were removed under reduced pressure and the resulting residue was partioned between EA and water. The aqueous layer was extracted with EA, the combined organic layers were dried over Na₂SO₄, filtered and concentrated to afford crude 4-[(2-nitroanilino)methyl]cyclohexanecarboxylate, which was purified by column chromatography (SiO₂; EA/heptane gradient), (860 mg, 2.95 mmol, 41% yield).

¹H NMR (600 MHz, DMSO-d₆): δ ppm 8.18 (br t, J=5.41 Hz, 1H), 8.06 (m, 1H), 7.52 (t, J=7.84 Hz, 1H), 7.07 (d, J=8.44 Hz, 1H), 6.67 (t, J=7.85 Hz, 1H), 3.58 (s, 3H), 3.27 (m, 2H), 2.27 (m, 1H), 1.93 (m, 2H), 1.82 (m, 2H), 1.63 (m, 1H), 1.32 (m, 2H), 1.06 (m, 2H).

### Step 2: Synthesis of Methyl trans-4-[(2-aminoanilino)methyl]cyclohexanecarboxylate—Compound Responding to Formula (VIa) as Defined in Scheme 2

A stirred suspension of methyl trans-4-(((2-nitrophenyl)amino)methyl)cyclohexane-1-carboxylate (862 mg, 2.95 mmol) in MeOH (70 ml) and Pd/C (10%, 54% water, 470 mg, 442 μmol) in a round bottom flask was evacuated and backfilled with H₂ at 0° C. This process was repeated 3 times. The suspension was vigorously stirred under a H₂ atmosphere (H₂ ballon) at rt for 2 hrs. The suspension was filtered, the filter cake rinsed with MeOH and the filtrate concentrated under reduced pressure. The title compound was obtained as a yellow solid and used in the next reaction without further purification (695 mg, 2.65 mmol, 90% yield).

¹H NMR (400 MHz, DMSO-d₆): δ ppm 6.49 (m, 2H), 6.38 (m, 2H), 4.47 (s, 2H), 4.32 (t, J=5.62 Hz, 1H), 3.58 (s, 3H), 2.86 (t, J=6.11 Hz, 2H), 2.27 (m, 1H), 1.92 (m, 4H), 1.55 (m, 1H), 1.31 (m, 2H), 1.00 (m, 2H).

### Step 3: Synthesis of Methyl trans-4-(benzimidazol-1-ylmethyl)cyclohexanecarboxylate—Corresponding to Compound (IIIa) in Scheme 2

To a stirred solution of methyl trans-4-(((2-aminophenyl)amino)methyl)cyclohexane-1-carboxylate (694 mg, 2.65 mmol) and trimethyl orthoformate (15 ml, 137 mmol) in MeOH (15 ml) conc. HCl (1.54 ml) was added at rt. The solution was stirred at rt for 1 hr. Volatile components were removed under reduced pressure and the resulting residue partitioned between saturated aqueous NaHCO₃ and EA. The aqueous layer was extracted with EA, the combined organic layers were dried over Na₂SO₄, filtered and concentrated to afford methyl trans-4-(benzimidazol-1-ylmethyl)cyclohexanecarboxylate, which was used in the next reaction without further purification (700 mg, 2.57 mmol, 97% yield).

¹H NMR (400 MHz, DMSO-d₆): δ ppm 8.18 (s, 1H), 7.64 (m, 2H), 7.22 (m, 2H), 4.10 (d, J=7.09 Hz, 2H), 3.56 (s, 3H), 2.24 (m, 1H), 1.85 (m, 3H), 1.58 (m, 2H), 1.25 (m, 2H), 1.07 (m, 2H).

### Step 4: Synthesis of trans-4-(benzimidazol-1-ylmethyl)cyclohexanecarboxylic Acid—Hydrolysis Step 2 in Scheme 1

To a solution of methyl trans-4-(benzimidazol-1-ylmethyl)cyclohexanecarboxylate (700 mg, 2.57 mmol) in MeOH (12 ml) and THF (12 ml) a solution of lithium hydroxide (185 mg, 7.71 mmol) in water (12 ml) was added. The resulting solution was stirred at rt for 3 hrs. Volatile components were removed under reduced pressure and the remaining aqueous solution acidified with 1 N HCl (10.0 ml, 10.0 mmol). The obtained solution was lyophilized. The title compound was obtained as a white solid (2×LiCl mixture), which was used in the next reaction without further purification (1.05 g, quant.).

¹H NMR (400 MHz, DMSO-d₆): δ ppm 12.04 (br s, 1H), 9.14 (s, 1H), 7.90 (d, J=7.21 Hz, 1H), 7.80 (d, J=7.27 Hz, 1H), 7.48 (m, 2H), 4.27 (d, J=7.21 Hz, 2H), 2.14 (m, 1H), 1.91 (m, 3H), 1.61 (m, 2H), 1.24 (m, 2H), 1.09 (m, 2H).

STEP 5a: Synthesis of trans-3-((S)-2-(4-((1H-benzo[d]imidazol-1-yl)methyl)cyclohexane-1-carbonyl)isoxazolidin-3-yl)-5-fluorobenzonitrile (Compound (7)) is performed as in STEP 5c of Example 1.4 here-below, but starting from trans-4-(benzimidazol-1-ylmethyl)cyclohexanecarboxylic acid instead of trans-4-[(5-cyano-6-fluoro-benzimidazol-1-yl)methyl]cyclohexanecarboxylic acid.

STEP 5b: Synthesis of trans-(4-((1H-benzo[d]imidazol-1-yl)methyl)cyclohexyl)((S)-3-(3,5-difluorophenyl)isoxazolidin-2-yl)methanone (Compound (8)) is performed as in STEP 5a here-above but starting from (3S)-3-(3,5-Difluorophenyl)isoxazolidine instead of 3-fluoro-5-[(3S)-isoxazolidin-3-yl]benzonitrile hydrochloride

STEP 5c: Synthesis of trans-(4-((1H-benzo[d]imidazol-1-yl)methyl)cyclohexyl)((S)-3-(5-fluoropyridin-3-yl)isoxazolidin-2-yl)methanone (Compound (9)) is performed as in STEP 5a here-above but starting from (3S)-3-(5-Fluoro-3-pyridyl)isoxazolidine of 3-fluoro-5-[(3S)-isoxazolidin-3-yl]benzonitrile hydrochloride.

### Example 1.2: Synthesis of Compounds (5), (6)

STEP 1: Synthesis of methyl trans-4-[(4-cyano-2-nitro-anilino)methyl]cyclohexanecarboxylate—Compound responding to formula (Va) as defined in Scheme 2 is performed as in STEP 1 of Example 1.1, but starting from 1-fluoro-2-nitro-4-benzonitrile.

STEP 2: Synthesis of methyl trans-4-[(2-amino-4-cyano-anilino)methyl]cyclohexanecarboxylate—Compound responding to formula (VIa) as defined in Scheme 2 is performed as in STEP 2 of Example 1.1, but starting from trans-4-[(4-cyano-2-nitro-anilino)methyl]cyclohexanecarboxylate.

STEP 3: Synthesis of methyl trans-4-[(5-cyanobenzimidazol-1-yl)methyl]cyclohexanecarboxylate—Corresponding to compound (IIIa) in Scheme 2 is performed as in STEP 3 of Example 1.1, but starting from methyl trans-4-[(2-amino-4-cyano-anilino)methyl]cyclohexanecarboxylate.

STEP 4: Synthesis of trans-4-[(5-cyanobenzimidazol-1-yl)methyl]cyclohexanecarboxylic acid in a similar way as performed in Example 1.1, but using methyl trans-4-[(5-cyanobenzimidazol-1-yl)methyl]cyclohexanecarboxylate instead of trans-4-(benzimidazol-1-ylmethyl)cyclohexanecarboxylate

STEP 5a: Synthesis of trans-1-((4-((S)-3-(3-cyano-5-fluorophenyl)isoxazolidine-2-carbonyl)cyclohexyl)methyl)-1H-benzo[d]imidazole-5-carbonitrile (Compound (5)) is performed as in STEP 5c of Example 1.4 below, but starting from trans-4-[(5-cyanobenzimidazol-1-yl)methyl]cyclohexanecarboxylic acid instead of trans-4-[(5-cyano-6-fluoro-benzimidazol-1-yl)methyl]cyclohexanecarboxylic acid.

STEP 5b: Synthesis of trans-1-((4-((S)-3-(5-fluoropyridin-3-yl)isoxazolidine-2-carbonyl)cyclohexyl)methyl)-1H-benzo[d]imidazole-5-carbonitrile (Compound (6)) is performed as in STEP 5a here-above but starting from (3S)-3-(5-Fluoro-3-pyridyl)isoxazolidine instead of 3-fluoro-5-[(3S)-isoxazolidin-3-yl]benzonitrile hydrochloride.

### Example 1.3: Synthesis of Compounds (12), (13), (39), (64), (98)

STEP 1: Synthesis of methyl trans-4-[(5-cyano-4-fluoro-2-nitro-anilino)methyl]cyclohexanecarboxylate—Compound responding to formula (Va) as defined in Scheme 2 is performed as Example 1.1, but starting from 5-cyano-1,4-difluoro-2-nitrobenzene.

STEP 2: Synthesis of methyl trans-4-[(2-amino-5-cyano-4-fluoro-anilino)methyl]cyclohexanecarboxylate—Compound responding to formula (VIa) as defined in Scheme 2 is performed as in Example 1.1, but starting from methyl trans-4-[(5-cyano-4-fluoro-2-nitro-anilino)methyl]cyclohexanecarboxylate.

STEP 3: Synthesis of methyl trans-4-[(6-cyano-5-fluoro-benzimidazol-1-yl)methyl]cyclohexanecarboxylate—Corresponding to compound (IIIa) in Scheme 2 is performed as in Example 1.1, but starting from methyl trans-4-[(2-amino-5-cyano-4-fluoro-anilino)methyl]cyclohexanecarboxylate.

STEP 4: Synthesis of trans-4-[(6-cyano-5-fluoro-benzimidazol-1-yl)methyl]cyclohexanecarboxylic acid—Compound (IIa) in Scheme 1—in a similar way as performed in Example 1.1, but using methyl trans-4-[(6-cyano-5-fluoro-benzimidazol-1-yl)methyl]cyclohexanecarboxylate instead of trans-4-(benzimidazol-1-ylmethyl)cyclohexanecarboxylate

STEP 5a: Synthesis of trans-1-((4-((S)-3-(5-cyanopyridin-3-yl)isoxazolidine-2-carbonyl)cyclohexyl)methyl)-5-fluoro-1H-benzo[d]imidazole-6-carbonitrile (Compound (64)).

To a mixture of 4-[(6-cyano-5-fluoro-benzimidazol-1-yl)methyl]cyclohexane carboxylic acid (25 mg, 83.0 μmol) and DMF (1.5 ml) was added Oxyma (37 mg, 0.25 mmol) and NaHCO₃ (63 mg, 0.75 mmol). The mixture was stirred for 1 hr at rt. 5-[(3S)-isoxazolidin-3-yl]pyridine-3-carbonitrile (15 mg, 85.6 μmol) in DMF (1.5 ml) was added. The mixture was warmed to 40° C. for 2 hrs. At rt, the mixture was diluted with EA and washed with water. The organic layer was separated, washed with NH₄Cl (saturated aqueous solution), NaHCO₃ (saturated aqueous solution) and brine, dried (Na₂SO₄), filtered and concentrated. The crude material was purified by preparative HPLC to afford the title compound (17 mg, 37.1 μmol, 45% yield).

STEP 5b: Synthesis of trans-5-fluoro-1-((4-((S)-3-(pyrazin-2-yl)isoxazolidine-2-carbonyl)cyclohexyl)methyl)-1H-benzo[d]imidazole-6-carbonitrile (Compound (98)) is performed as STEP 5a here-above, but starting from (3S)-3-Pyrazin-2-ylisoxazolidine hydrochloride salt instead of 5-[(3S)-isoxazolidin-3-yl]pyridine-3-carbonitrile.

STEP 5c: Synthesis of trans-1-((4-((S)-3-(3,5-difluorophenyl)isoxazolidine-2-carbonyl)cyclohexyl)methyl)-5-fluoro-1H-benzo[d]imidazole-6-carbonitrile (Compound (12)) is performed as in STEP 5c of Example 1.4, but starting from trans-4-[(6-cyano-5-fluoro-benzimidazol-1-yl)methyl]cyclohexanecarboxylic acid and (3S)-3-(3,5-difluorophenyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

STEP 5d: Synthesis of trans-1-((4-((S)-3-(3-cyano-5-fluorophenyl)isoxazolidine-2-carbonyl)cyclohexyl)methyl)-5-fluoro-1H-benzo[d]imidazole-6-carbonitrile (Compound (13)) is performed as in STEP 5c of Example 1.4, but starting from trans-4-[(6-cyano-5-fluoro-benzimidazol-1-yl)methyl]cyclohexanecarboxylic acid instead of trans-4-[(5-cyano-6-fluoro-benzimidazol-1-yl)methyl]cyclohexanecarboxylic acid.

STEP 5e: Synthesis of trans-5-fluoro-1-((4-((S)-3-(5-methylfuran-3-yl)isoxazolidine-2-carbonyl)cyclohexyl)methyl)-1H-benzo[d]imidazole-6-carbonitrile (Compound (39)) is performed as in STEP 5d here-above but starting from (3S)-3-(5-Methyl-3-furyl)isoxazolidine instead of 3-fluoro-5-[(3S)-isoxazolidin-3-yl]benzonitrile hydrochloride.

### Example 1.4: Synthesis of Compounds (10), (11), (40), (65), (97)

STEP 1: Synthesis of methyl trans-4-[(4-cyano-5-fluoro-2-nitro-anilino)methyl]cyclohexanecarboxylate—Compound responding to formula (Va) as defined in Scheme 2 is performed as in Example 1.1, but starting from 4-cyano-1,5-difluoro-2-nitrobenzene

STEP 2: Synthesis of methyl trans-4-[(2-amino-4-cyano-5-fluoro-anilino)methyl]cyclohexanecarboxylate—Compound responding to formula (VIa) as defined in Scheme 2 is performed as in Example 1.1, but starting from methyl trans-4-[(4-cyano-5-fluoro-2-nitro-anilino)methyl]cyclohexanecarboxylate

STEP 3: Synthesis of methyl trans-4-[(5-cyano-6-fluoro-benzimidazol-1-yl)methyl]cyclohexanecarboxylate—Corresponding to compound (IIIa) in Scheme 2 is performed as in Example 1.1, but starting from methyl trans-4-[(2-amino-4-cyano-5-fluoro-anilino)methyl]cyclohexanecarboxylate

STEP 4: Synthesis of trans-4-[(5-cyano-6-fluoro-benzimidazol-1-yl)methyl]cyclohexanecarboxylic acid in a similar way as performed in Example 1.1, but using methyl trans-4-[(5-cyano-6-fluoro-benzimidazol-1-yl)methyl]cyclohexanecarboxylate instead of trans-4-(benzimidazol-1-ylmethyl)cyclohexanecarboxylate

STEP 5a: Synthesis of trans-1-((4-((S)-3-(5-cyanopyridin-3-yl)isoxazolidine-2-carbonyl)cyclohexyl)methyl)-6-fluoro-1H-benzo[d]imidazole-5-carbonitrile (Compound (65)) is performed as STEP 5a of Example 1.3, but starting from trans-4-[(5-cyano-6-fluoro-benzimidazol-1-yl)methyl]cyclohexanecarboxylic acid and 5-[(3S)-Isoxazolidin-3-yl]pyridine-3-carbonitrile instead of trans-4-[(6-cyano-5-fluoro-benzimidazol-1-yl)methyl]cyclohexanecarboxylic acid and 5-[(3S)-isoxazolidin-3-yl]pyridine-3-carbonitrile respectively.

STEP 5b: Synthesis of trans-6-fluoro-1-((4-((S)-3-(pyrazin-2-yl)isoxazolidine-2-carbonyl)cyclohexyl)methyl)-1H-benzo[d]imidazole-5-carbonitrile (Compound (97)) is performed as STEP 5a here-above, but starting from (3S)-3-Pyrazin-2-ylisoxazolidine hydrochloride salt instead of 5-[(3S)-Isoxazolidin-3-yl]pyridine-3-carbonitrile.

STEP 5c: Synthesis of trans-1-((4-((S)-3-(3-cyano-5-fluorophenyl)isoxazolidine-2-carbonyl)cyclohexyl)methyl)-6-fluoro-1H-benzo[d]imidazole-5-carbonitrile (Compound (11)) To a stirred solution of trans-4-[(5-cyano-6-fluoro-benzimidazol-1-yl)methyl]cyclohexane carboxylic acid (100 mg, 331 μmol), NEtiPr₂ (174 μl, 995 μmol) and 3-fluoro-5-[(3S)-isoxazolidin-3-yl]benzonitrile hydrochloride (98.7 mg, 431 μmol) in DMF (2 ml) HATU (151 mg, 398 μmol) was added at rt. Stirring at rt was continued for 2 hrs. The reaction mixture was filtered and subjected to preparative reversed phase HPLC (70 mg, 147 μmol, 44% yield).

STEP 5d: Synthesis of trans-1-((4-((S)-3-(3,5-difluorophenyl)isoxazolidine-2-carbonyl)cyclohexyl)methyl)-6-fluoro-1H-benzo[d]imidazole-5-carbonitrile (Compound (10)) is performed as in STEP 5c here-above but starting from (3S)-3-(3,5-Difluorophenyl)isoxazolidine instead of 3-fluoro-5-[(3S)-isoxazolidin-3-yl]benzonitrile hydrochloride.

STEP 5e: Synthesis of trans-6-fluoro-1-((4-((S)-3-(5-methylfuran-3-yl)isoxazolidine-2-carbonyl)cyclohexyl)methyl)-1H-benzo[d]imidazole-5-carbonitrile (Compound (40)) is performed as in STEP 5c here-above but starting from (3S)-3-(5-Methyl-3-furyl)isoxazolidine instead of 3-fluoro-5-[(3S)-isoxazolidin-3-yl]benzonitrile hydrochloride.

### Example 1.5: Synthesis of Compounds (36), (37), (38)

STEP 1: Synthesis of methyl trans-4-[(5-cyano-3-fluoro-2-nitro-anilino)methyl]cyclohexanecarboxylate—Compound responding to formula (Va) as defined in Scheme 2 is performed as in Example 1.1, but starting from 5-cyano-1,3-difluoro-2-nitrobenzene

STEP 2: Synthesis of methyl trans-4-[(2-amino-5-cyano-3-fluoro-anilino)methyl]cyclohexanecarboxylate—Compound responding to formula (VIa) as defined in Scheme 2 is performed as in Example 1.1, but starting from methyl trans-4-[(5-cyano-3-fluoro-2-nitro-anilino)methyl]cyclohexanecarboxylate

STEP 3: Synthesis of methyl trans-4-[(6-cyano-4-fluoro-benzimidazol-1-yl)methyl]cyclohexanecarboxylate—Corresponding to compound (IIIa) in Scheme 2 is performed as in Example 1.1, but starting from methyl trans-4-[(2-amino-5-cyano-3-fluoro-anilino)methyl]cyclohexanecarboxylate

STEP 4: Synthesis of trans-4-[(6-cyano-4-fluoro-benzimidazol-1-yl)methyl]cyclohexanecarboxylic acid—Compound (IIa) in Scheme 1—in a similar way as performed in Example 1.1, but using methyl trans-4-[(6-cyano-4-fluoro-benzimidazol-1-yl)methyl]cyclohexanecarboxylate instead of trans-4-(benzimidazol-1-ylmethyl)cyclohexanecarboxylate

STEP 5a: Synthesis of trans-1-((4-((S)-3-(3-cyano-5-fluorophenyl)isoxazolidine-2-carbonyl)cyclohexyl)methyl)-4-fluoro-1H-benzo[d]imidazole-6-carbonitrile (Compound (36)) is performed as in STEP 5c of Example 1.4, but starting from trans-4-[(6-cyano-4-fluoro-benzimidazol-1-yl)methyl]cyclohexanecarboxylic acid instead of trans-4-[(5-cyano-6-fluoro-benzimidazol-1-yl)methyl]cyclohexanecarboxylic acid.

STEP 5b: Synthesis of trans-1-((4-((S)-3-(3,5-difluorophenyl)isoxazolidine-2-carbonyl)cyclohexyl)methyl)-4-fluoro-1H-benzo[d]imidazole-6-carbonitrile (Compound (37)) is performed as in STEP 5a here-above but starting from (3S)-3-(3,5-Difluorophenyl)isoxazolidine instead of 3-fluoro-5-[(3S)-isoxazolidin-3-yl]benzonitrile hydrochloride.

STEP 5c: Synthesis of trans-4-fluoro-1-((4-((S)-3-(5-methylfuran-3-yl)isoxazolidine-2-carbonyl)cyclohexyl)methyl)-1H-benzo[d]imidazole-6-carbonitrile (Compound (38)) is performed as in STEP 5a here-above but starting from (3S)-3-(5-Methyl-3-furyl)isoxazolidine instead of 3-fluoro-5-[(3S)-isoxazolidin-3-yl]benzonitrile hydrochloride.

### Example 1.6: Synthesis of Compounds (48), (49), (50), (51), (92), (106), (107), (112), (113)

STEP 1: Synthesis of methyl trans-4-[(4-fluoro-2-nitro-anilino)methyl]cyclohexanecarboxylate—Compound responding to formula (Va) as defined in Scheme 2 is performed as in Example 1.1, but starting from 1,4-difluoro-2-nitrobenzene

STEP 2: Synthesis of methyl trans-4-[(2-amino-4-fluoro-anilino)methyl]cyclohexanecarboxylate—Compound responding to formula (VIa) as defined in Scheme 2 is performed as in Example 1.1, but starting from methyl trans-4-[(4-fluoro-2-nitro-anilino)methyl]cyclohexanecarboxylate

### For Compounds (48), (49), (92), (107)

STEP 3: Synthesis of methyl trans-4-[(5-fluorobenzimidazol-1-yl)methyl]cyclohexanecarboxylate—Corresponding to compound (IIIa) in Scheme 2 is performed as in Example 1.1, but starting from methyl trans-4-[(4-fluoro-2-nitro-anilino)methyl]cyclohexanecarboxylate

STEP 4: Synthesis of trans-4-[(5-fluorobenzimidazol-1-yl)methyl]cyclohexanecarboxylic acid—Compound (IIa) in Scheme 1—in a similar way as performed in Example 1.1, but using methyl trans-4-[(5-fluorobenzimidazol-1-yl)methyl]cyclohexanecarboxylate instead of trans-4-(benzimidazol-1-ylmethyl)cyclohexanecarboxylate

STEP 5a: Synthesis of trans-(4-((5-fluoro-1H-benzo[d]imidazol-1-yl)methyl)cyclohexyl)((S)-3-(pyrazin-2-yl)isoxazolidin-2-yl)methanone (Compound (92)) is performed as in STEP 5a of Example 1.3, but starting from trans-4-[(5-fluorobenzimidazol-1-yl)methyl]cyclohexanecarboxylic acid and (3S)-3-Pyrazin-2-ylisoxazolidine hydrochloride salt instead of trans-4-[(6-cyano-5-fluoro-benzimidazol-1-yl)methyl]cyclohexanecarboxylic acid and 5-[(3S)-isoxazolidin-3-yl]pyridine-3-carbonitrile respectively.

STEP 5b: Synthesis of trans-5-((S)-2-(4-((5-fluoro-1H-benzo[d]imidazol-1-yl)methyl)cyclohexane-1-carbonyl)isoxazolidin-3-yl)nicotinonitrile (Compound (107)) is performed as in STEP 5a here-above but starting from 5-[(3S)-Isoxazolidin-3-yl]pyridine-3-carbonitrile instead of (3S)-3-Pyrazin-2-ylisoxazolidine hydrochloride salt.

STEP 5c: Synthesis of trans-3-fluoro-5-((S)-2-(4-((5-fluoro-1H-benzo[d]imidazol-1-yl)methyl)cyclohexane-1-carbonyl)isoxazolidin-3-yl)benzonitrile (Compound (48)) is performed as in STEP 5c of Example 1.4, but starting from trans-4-[(5-fluorobenzimidazol-1-yl)methyl]cyclohexanecarboxylic acid instead of trans-4-[(5-cyano-6-fluoro-benzimidazol-1-yl)methyl]cyclohexanecarboxylic acid.

STEP 5d: Synthesis of trans-((S)-3-(3,5-difluorophenyl)isoxazolidin-2-yl)(4-((5-fluoro-1H-benzo[d]imidazol-1-yl)methyl)cyclohexyl)methanone (Compound (49)) is performed as in STEP 5c here-above but starting from (3S)-3-(3,5-Difluorophenyl)isoxazolidine instead of 3-fluoro-5-[(3S)-isoxazolidin-3-yl]benzonitrile hydrochloride.

### For Compound (106), (112), (113)

STEP 3: Synthesis of methyl trans-4-[(6-fluorobenzimidazol-1-yl)methyl]cyclohexanecarboxylate—Corresponding to compound (IIIa) in Scheme 2 is performed as in Example 1.1, but starting from methyl trans-4-[(4-fluoro-2-nitro-anilino)methyl]cyclohexanecarboxylate

STEP 4: Synthesis of trans-4-[(6-fluorobenzimidazol-1-yl)methyl]cyclohexanecarboxylic acid—Compound (IIa) in Scheme 1—in a similar way as performed in Example 1.1, but using methyl trans-4-[(6-fluorobenzimidazol-1-yl)methyl]cyclohexanecarboxylate instead of trans-4-(benzimidazol-1-ylmethyl)cyclohexanecarboxylate

STEP 5a: Synthesis of trans-5-((S)-2-(4-((6-fluoro-1H-benzo[d]imidazol-1-yl)methyl)cyclohexane-1-carbonyl)isoxazolidin-3-yl)nicotinonitrile (Compound (106)) is performed as in STEP 5a of Example 1.3, but starting from trans-4-[(6-fluorobenzimidazol-1-yl)methyl]cyclohexanecarboxylic acid and 5-[(3S)-Isoxazolidin-3-yl]pyridine-3-carbonitrile instead of trans-4-[(6-cyano-5-fluoro-benzimidazol-1-yl)methyl]cyclohexanecarboxylic acid and 5-[(3S)-isoxazolidin-3-yl]pyridine-3-carbonitrile respectively.

STEP 5b: Synthesis of trans-3-fluoro-5-((S)-2-(4-((6-fluoro-1H-benzo[d]imidazol-1-yl)methyl)cyclohexane-1-carbonyl)isoxazolidin-3-yl)benzonitrile (Compound (112)) is performed as in STEP 5c of Example 1.4, but starting from trans-4-[(6-fluorobenzimidazol-1-yl)methyl]cyclohexanecarboxylic acid instead of trans-4-[(5-cyano-6-fluoro-benzimidazol-1-yl)methyl]cyclohexanecarboxylic acid.

STEP 5c: Synthesis of trans-((S)-3-(3,5-difluorophenyl)isoxazolidin-2-yl)(4-((6-fluoro-1H-benzo[d]imidazol-1-yl)methyl)cyclohexyl)methanone (Compound (113)) is performed as in STEP 5b here-above but starting from (3S)-3-(3,5-Difluorophenyl)isoxazolidine instead of 3-fluoro-5-[(3S)-isoxazolidin-3-yl]benzonitrile hydrochloride.

### For Compounds (50), (51)

STEP 3: Synthesis of methyl trans-4-[(5-fluoro-2-methyl-benzimidazol-1-yl)methyl]cyclohexanecarboxylate—Corresponding to compound (IIIa) in Scheme 2

To a suspension of methyl trans-4-(((2-amino-4-fluorophenyl)amino)methyl)cyclo-hexane-1-carboxylate (650 mg, 2.32 mmol, 1 eq.), in dioxane (5 ml) was added acetyl chloride (334 μl, 4.64 mmol, 2 eq.). The resulting solution was heated at 70° C. for 8 hrs. The obtained suspension was cooled to rt and filtered. The filter cake was suspended in EA and washed with sat. NaHCO₃ solution. The organic layer was dried over Na₂SO₄, filtered and concentrated in vacuo to give 557 mg (79% yield) of the title compound as a brown oil.

STEP 4: Synthesis of trans-4-[(5-fluoro-2-methyl-benzimidazol-1-yl)methyl]cyclohexanecarboxylic acid in a similar way as performed in Example 1.1, but using methyl trans-4-[(5-fluoro-2-methyl-benzimidazol-1-yl)methyl]cyclohexanecarboxylate instead of trans-4-(benzimidazol-1-ylmethyl)cyclohexanecarboxylate

STEP 5a: Synthesis of trans-3-fluoro-5-((S)-2-(4-((5-fluoro-2-methyl-1H-benzo[d]imidazol-1-yl)methyl)cyclohexane-1-carbonyl)isoxazolidin-3-yl)benzonitrile (Compound (50)) is performed as in STEP 5c of Example 1.4, but starting from trans-4-[(5-fluoro-2-methyl-benzimidazol-1-yl)methyl]cyclohexanecarboxylic acid instead of trans-4-[(5-cyano-6-fluoro-benzimidazol-1-yl)methyl]cyclohexanecarboxylic acid.

STEP 5b: Synthesis of trans-((S)-3-(3,5-difluorophenyl)isoxazolidin-2-yl)(4-((5-fluoro-2-methyl-1H-benzo[d]imidazol-1-yl)methyl)cyclohexyl)methanone (Compound (51)) is performed as in STEP 5a here-above but starting from (3S)-3-(3,5-Difluorophenyl)isoxazolidine instead of 3-fluoro-5-[(3S)-isoxazolidin-3-yl]benzonitrile hydrochloride.

### Example 1.7: Synthesis of Compounds (114), (115), (116)

STEP 1: Synthesis of methyl trans-3-[(4-fluoro-2-nitro-anilino)methyl]cyclobutanecarboxylate—Compound responding to formula (Va) as defined in Scheme 2 is performed as in Example 1.1, but starting from 1,4-difluoro-2-nitrobenzene and methyl trans-3-[(amino)methyl]cyclobutanecarboxylate

STEP 2: Synthesis of methyl trans-3-[(2-amino-4-fluoro-anilino)methyl]cyclobutanecarboxylate—Compound responding to formula (VIa) as defined in Scheme 2 is performed as in Example 1.1, but starting from methyl trans-3-[(4-fluoro-2-nitro-anilino)methyl]cyclobutanecarboxylate

STEP 3: Synthesis of methyl trans-3-[(5-fluorobenzimidazol-1-yl)methyl]cyclobutanecarboxylate—Corresponding to compound (IIIa) in Scheme 2 is performed as in Example 1.1, but starting from methyl trans-3-[(2-amino-4-fluoro-anilino)methyl]cyclobutanecarboxylate

STEP 4: Synthesis of trans-3-[(5-fluorobenzimidazol-1-yl)methyl]cyclobutanecarboxylic acid—Compound (IIa) in Scheme 1—in a similar way as performed in Example 1.1, but using methyl trans-3-[(5-fluorobenzimidazol-1-yl)methyl]cyclobutanecarboxylate instead of trans-4-(benzimidazol-1-ylmethyl)cyclohexanecarboxylate

STEP 5a: Synthesis of trans-3-fluoro-5-((S)-2-(3-((5-fluoro-1H-benzo[d]imidazol-1-yl)methyl)cyclobutane-1-carbonyl)isoxazolidin-3-yl)benzonitrile (Compound (116)) is performed as in STEP 5c of Example 1.4, but starting from trans-3-[(5-fluorobenzimidazol-1-yl)methyl]cyclobutanecarboxylic acid instead of trans-4-[(5-cyano-6-fluoro-benzimidazol-1-yl)methyl]cyclohexanecarboxylic acid.

STEP 5b: Synthesis of trans-(3-((5-fluoro-1H-benzo[d]imidazol-1-yl)methyl)cyclobutyl)((S)-3-(p-tolyl)isoxazolidin-2-yl)methanone (Compound (114)) is performed as in STEP 5a here-above but starting from (3S)-3-(4-methylphenyl)isoxazolidine instead of 3-fluoro-5-[(3S)-isoxazolidin-3-yl]benzonitrile hydrochloride.

STEP 5c: Synthesis of trans-((S)-3-(3,5-difluorophenyl)isoxazolidin-2-yl)(3-((5-fluoro-1H-benzo[d]imidazol-1-yl)methyl)cyclobutyl)methanone (Compound (115)) is performed as in STEP 5a here-above but starting from (3S)-3-(3,5-Difluorophenyl)isoxazolidine instead of 3-fluoro-5-[(3S)-isoxazolidin-3-yl]benzonitrile hydrochloride.

### Example 2: Synthesis of Compounds (66), (67), (68), (69), (84), (85), (86), (87), (100), (101)

### Example 2.1: Synthesis of Compounds (86), (87)

### STEP 1: Synthesis of 5-fluoro-1H-indazole-6-carbonitrile—Heteroaromatic Compound Containing a Nitrogen Atom Used in Step 6a of Scheme 3

Through a suspension in a micro wave vial of ZnCN₂ (521 mg, 4.39 mmol), 5-bromo-6-fluoro-1H-indazole (900 mg, 4.19 mmol), Pd₂(dba)₃ (200 mg, 209 μmol) and S-PHOS (175 mg, 418 μmol) in DMF (20 ml) and water (0.1 ml) Ar was bubbled for 10 min via a syringe at rt. In a micro wave the mixture was heated to 150° C. for 30 min. The suspension was allowed to reach rt and diluted with EA. The resulting suspension was filtered and the filtrate diluted with water. The aqueous layer was extracted with EA and the combined organic layers were washed with water (3×), dried over Na₂SO₄, filtered and concentrated to afford the crude title compound as pale yellow solid, which was triturated with CH₂Cl₂. The suspension was filtered and the filter cake rinsed with little CH₂Cl₂. 5-fluoro-1H-indazole-6-carbonitrile was obtained as a pale yellow solid (370 mg, 2.30 mmol, 55% yield).

¹H NMR (400 MHz, DMSO-da): δ ppm 13.7 (s, 1H), 8.50 (d, J=6.21 Hz, 1H), 8.30 (s, 1H) 7.65 (d, J=9.98 Hz, 1H).

STEP 2: Synthesis of the corresponding carboxylate methyl trans-4-[(6-cyano-5-fluoro-indazol-1-yl)methyl]cyclohexanecarboxylate in a similar way as performed in example 3.1 step 1.

STEP 3: Synthesis of trans-4-[(6-cyano-5-fluoro-indazol-1-yl)methyl]cyclohexanecarboxylic acid—Compound (IIa) in Scheme 1—in a similar way as performed in Example 1.1, but using methyl trans-4-[(6-cyano-5-fluoro-indazol-1-yl)methyl]cyclohexanecarboxylate instead of trans-4-(benzimidazol-1-ylmethyl)cyclohexanecarboxylate

STEP 4a: Synthesis of trans-1-((4-((S)-3-(3-cyano-5-fluorophenyl)isoxazolidine-2-carbonyl)cyclohexyl)methyl)-5-fluoro-1H-indazole-6-carbonitrile (Compound (87)) is performed as in STEP 5c of Example 1.4, but starting from trans-4-[(6-cyano-5-fluoro-indazol-1-yl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-1-((4-((S)-3-(3,5-difluorophenyl)isoxazolidine-2-carbonyl)cyclohexyl)methyl)-5-fluoro-1H-indazole-6-carbonitrile (Compound (86)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

### Example 2.2: Synthesis of Compounds (66), (67)

STEP 1: Synthesis of 6-fluoro-1H-indazole-5-carbonitrile—heteroaromatic compound containing a Nitrogen atom used in step 6a of Scheme 3 in a similar way as performed in example 2.1 step 1, but starting from 5-bromo-6-fluoro-1H-indazole.

STEP 2: Synthesis of the corresponding carboxylate methyl trans-4-[(5-cyano-6-fluoro-indazol-1-yl)methyl]cyclohexanecarboxylate in a similar way as performed in example 3.1 step 1.

STEP 3: Synthesis of trans-4-[(5-cyano-6-fluoro-indazol-1-yl)methyl]cyclohexanecarboxylic acid—Compound (IIa) in Scheme 1—in a similar way as performed in Example 1.1, but using methyl trans-4-[(5-cyano-6-fluoro-indazol-1-yl)methyl]cyclohexanecarboxylate instead of trans-4-(benzimidazol-1-ylmethyl)cyclohexanecarboxylate

STEP 4a: Synthesis of trans-1-((4-((S)-3-(3-cyano-5-fluorophenyl)isoxazolidine-2-carbonyl)cyclohexyl)methyl)-6-fluoro-1H-indazole-5-carbonitrile (Compound (66)) is performed as in STEP 5c of Example 1.4, but starting from trans-4-[(5-cyano-6-fluoro-indazol-1-yl)methyl]cyclohexanecarboxylic add instead of trans-4-[(5-cyano-6-fluoro-benzimidazol-1-yl)methyl]cyclohexanecarboxylic acid.

STEP 4b: Synthesis of trans-1-((4-((S)-3-(3,5-difluorophenyl)isoxazolidine-2-carbonyl)cyclohexyl)methyl)-6-fluoro-1H-indazole-5-carbonitrile (Compound (67)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

### Example 2.3: Synthesis of Compounds (68), (69)

STEP 1: Synthesis of 6-fluoro-3-methyl-1H-indazole-5-carbonitrile—heteroaromatic compound containing a Nitrogen atom used in step 6a of Scheme 3 in a similar way as performed in example 2.1 step 1, but starting from 5-bromo-6-fluoro-3-methyl-1H-indazole

STEP 2: Synthesis of the corresponding carboxylate methyl trans-4-[(5-cyano-6-fluoro-3-methyl-indazol-1-yl)methyl]cyclohexanecarboxylate in a similar way as performed in example 3.1 step 1.

STEP 3: Synthesis of trans-4-[(5-cyano-6-fluoro-3-methyl-indazol-1-yl)methyl]cyclohexanecarboxylic acid—Compound (IIa) in Scheme 1—in a similar way as performed in Example 1.1, but using methyl trans-4-[(5-cyano-6-fluoro-3-methyl-indazol-1-yl)methyl]cyclohexanecarboxylate instead of trans-4-(benzimidazol-1-ylmethyl)cyclohexanecarboxylate

STEP 4a: Synthesis of trans-1-((4-((S)-3-(3-cyano-5-fluorophenyl)isoxazolidine-2-carbonyl)cyclohexyl)methyl)-6-fluoro-3-methyl-1H-indazole-5-carbonitrile (Compound (69)) is performed as in STEP 5c of Example 1.4, but starting from trans-4-[(5-cyano-6-fluoro-3-methyl-indazol-1-yl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-1-((4-((S)-3-(3,5-difluorophenyl)isoxazolidine-2-carbonyl)cyclohexyl)methyl)-6-fluoro-3-methyl-1H-indazole-5-carbonitrile (Compound (68)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

### Example 2.4: Synthesis of Compounds (84), (85)

STEP 1: Synthesis of 5-fluoro-3-methyl-1H-indazole-6-carbonitrile—heteroaromatic compound containing a Nitrogen atom used in step 6a of Scheme 3 in a similar way as performed in example 2.1 step 1, but starting from 6-bromo-5-fluoro-3-methyl-1H-indazole

STEP 2: Synthesis of the corresponding carboxylate methyl trans-4-[(6-cyano-5-fluoro-3-methyl-indazol-1-yl)methyl]cyclohexanecarboxylate in a similar way as performed in example 3.1 step 1.

STEP 3: Synthesis of trans-4-[(6-cyano-5-fluoro-3-methyl-indazol-1-yl)methyl]cyclohexanecarboxylic acid—Compound (IIa) in Scheme 1—in a similar way as performed in Example 1.1, but using methyl trans-4-[(6-cyano-5-fluoro-3-methyl-indazol-1-yl)methyl]cyclohexanecarboxylate instead of trans-4-(benzimidazol-1-ylmethyl)cyclohexanecarboxylate

STEP 4a: Synthesis of trans-1-((4-((S)-3-(3-cyano-5-fluorophenyl)isoxazolidine-2-carbonyl)cyclohexyl)methyl)-5-fluoro-3-methyl-1H-indazole-6-carbonitrile (Compound (85)) is performed as in STEP 5c of Example 1.4, but starting from trans-4-[(6-cyano-5-fluoro-3-methyl-indazol-1-yl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-1-((4-((S)-3-(3,5-difluorophenyl)isoxazolidine-2-carbonyl)cyclohexyl)methyl)-5-fluoro-1H-indazole-6-carbonitrile (Compound (84)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

### Example 2.5: Synthesis of Compounds (100), (101)

STEP 1: Synthesis of 4-fluoro-3-methyl-1H-indazole-6-carbonitrile—heteroaromatic compound containing a Nitrogen atom used in step 6a of Scheme 3 in a similar way as performed in example 2.1 step 1, but starting from 6-bromo-4-fluoro-3-methyl-1H-indazole.

STEP 2: Synthesis of the corresponding carboxylate methyl trans-4-[(6-cyano-4-fluoro-3-methyl-indazol-1-yl)methyl]cyclohexanecarboxylate in a similar way as performed in example 3.1 step 1.

STEP 3: Synthesis of trans-4-[(6-cyano-4-fluoro-3-methyl-indazol-1-yl)methyl]cyclohexanecarboxylic acid—Compound (IIa) in Scheme 1—in a similar way as performed in Example 1.1, but using methyl trans-4-[(6-cyano-4-fluoro-3-methyl-indazol-1-yl)methyl]cyclohexanecarboxylate instead of trans-4-(benzimidazol-1-ylmethyl)cyclohexanecarboxylate

STEP 4a: Synthesis of trans-1-((4-((S)-3-(3-cyano-5-fluorophenyl)isoxazolidine-2-carbonyl)cyclohexyl)methyl)-4-fluoro-3-methyl-1H-indazole-6-carbonitrile (Compound (100)) is performed as in STEP 5c of Example 1.4, but starting from trans-4-[(6-cyano-4-fluoro-3-methyl-indazol-1-yl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-1-((4-((S)-3-(3,5-difluorophenyl)isoxazolidine-2-carbonyl)cyclohexyl)methyl)-4-fluoro-3-methyl-1H-indazole-6-carbonitrile (Compound (101)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

### Example 3: Synthesis of Compounds (2), (3), (14), (15), (16), (17), (18), (19), (20), (21), (22), (23), (24), (25), (26), (27), (28), (29), (30), (31), (32), (33), (34), (35), (41), (42), (43), (44), (45), (46), (47), (52), (53), (54), (55), (56), (57), (58), (59), (60), (61), (62), (63), (82), (83), (88), (89), (90), (91), (93), (94), (95), (96), (104), (105), (108), (110), (111), (117), (118), (119), (120), (121), (123), (126), (127), (128), (130), (134), (137), (185), (186), (187), (188), (189), (190), (196), (198), (199), (200), (202), (203), (204), (215), (218) and (241) Synthesis of Methyl trans-4-(iodomethyl)cyclohexanecarboxylate

To a stirred solution of methyl trans-4-(methylsulfonyloxymethyl)cyclohexane-carboxylate (6.43 g, 25.7 mmol) in acetone (100 ml) NaI (11.0 g, 73.2 mmol) was added at rt. The solution was heated at reflux for 6 hrs. The suspension was cooled to rt and diluted with Et₂O. The suspension was filtered and the filtrate concentrated under reduced pressure. The obtained crude yellow solid was again triturated with Et₂O, filtered again and the filtrate concentrated under reduced pressure. The title compound was obtained as a pale yellow oil, which was used in the next reaction without further purification (6.08 g, 21.5 mmol, 84% yield).

¹H NMR (400 MHz, CDCl₃): δ ppm 3.67 (s, 3H), 3.11 (d, J=7.21 Hz, 2H), 3.01 (s, 3H) 2.23 (m, 1H), 2.05 (m, 4H), 1.45 (m, 3H), 1.03 (m, 2H).

### Example 3.1: Synthesis of Compounds (28), (29), (30), (31), (41), (43), (127), (189), (190), (196), (200), (203), (204), (215)

### STEP 1: Synthesis of Methyl trans-4-[(6-cyanoindazol-1-yl)methyl]cyclohexanecarboxylate and Methyl trans-4-[(6-cyanoindazol-2-yl)methyl]cyclohexanecarboxylate—Heteroaromatic Compounds Containing a Nitrogen Atom Used in Step 6a of Scheme 3

To a stirred solution of 1H-indazole-6-carbonitrile (200 mg, 1.40 mmol) in 1 ml DMF NaH (33.5 mg, 1.40 mmol) was added and stirring was continued for 10 min at rt. A solution of methyl trans-4-(iodomethyl)cyclohexanecarboxylate (1.11 g, 3.93 mmol) in 2 ml DMF was added and the resulting suspension was stirred 1.5 hrs at rt before another portion of NaH (33.5 mg, 1.40 mmol) was added. The mixture was stirred for additional 1.5 hrs at rt. The reaction was quenched with water at 0° C. and the aqueous layer extracted with MTBE, the combined organic layers were dried over Na₂SO₄, filtered and concentrated to afford the crude title compounds, which were purified by column chromatography (SiO₂; EA/heptane gradient).

methyl trans-4-[(6-cyanoindazol-1-yl)methyl]cyclohexanecarboxylate (196 mg, 660 μmol, 47% yield).

¹H NMR (400 MHz, DMSO-d₆): δ ppm 8.45 (s, 1H), 8.25 (s, 1H), 7.97 (d, J=8.31 Hz, 1H), 7.45 (d, J=8.31, 1H), 4.34 (d, J=7.21 Hz, 2H), 3.56 (s, 3H), 2.23 (m, 1H), 1.89 (m, 3H), 1.52 (m, 2H), 1.25 (m, 2H), 1.10 (m, 2H).

methyl trans-4-[(6-cyanoindazol-2-yl)methyl]cyclohexanecarboxylate (124 mg, 417 μmol, 30% yield).

¹H NMR (400 MHz, DMSO-d₆): δ ppm 8.55 (s, 1H), 8.30 (s, 1H), 7.92 (d, J=8.64 Hz, 1H), 7.28 (d, J=8.62, 1H), 4.36 (d, J=7.09 Hz, 2H), 3.57 (s, 3H), 2.25 (m, 1H), 1.92 (m, 3H), 1.57 (m, 2H), 1.28 (m, 2H), 1.09 (m, 2H).

STEP 2: Synthesis of trans-4-[(6-cyanoindazol-1-yl)methyl]cyclohexanecarboxylic acid and trans-4-[(6-cyanoindazol-2-yl)methyl]cyclohexanecarboxylic acid—Compounds (Iia) in Scheme 1—in a similar way as performed in Example 1.1, but using methyl trans-4-[(6-cyanoindazol-1-yl)methyl]cyclohexanecarboxylate and methyl trans-4-[(6-cyanoindazol-2-yl)methyl]cyclohexanecarboxylate respectively instead of trans-4-(benzimidazol-1-ylmethyl)cyclohexanecarboxylate

STEP 3a: Synthesis of trans-1-((4-((S)-3-(3-cyano-5-fluorophenyl)isoxazolidine-2-carbonyl)cyclohexyl)methyl)-1H-indazole-6-carbonitrile (Compound (28)) is performed as in STEP 5c of Example 1.4, but starting from trans-4-[(6-cyanoindazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-1H-indazole-6-carbonitrile (Compound (29)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3c: Synthesis of trans-2-((4-((S)-3-(3-cyano-5-fluorophenyl)isoxazolidine-2-carbonyl)cyclohexyl)methyl)-2H-indazole-6-carbonitrile (Compound (30)) is performed as in STEP 5c of Example 1.4, but starting from trans-4-[(6-cyanoindazol-2-yl)methyl]cyclohexanecarboxylic acid instead of trans-4-[(5-cyano-6-fluoro-benzimidazol-1-yl)methyl]cyclohexanecarboxylic acid.

STEP 3d: Synthesis of trans-2-((4-((S)-3-(3,5-difluorophenyl)isoxazolidine-2-carbonyl)cyclohexyl)methyl)-2H-indazole-6-carbonitrile (Compound (31)) is performed as in STEP 3c here-above but starting from (3S)-3-(3-cyano-5-fluorophenyl)isoxazolidine instead of 3-fluoro-5-[(3S)-isoxazolidin-3-yl]benzonitrile hydrochloride.

STEP 3e: Synthesis of trans-2-((4-((S)-3-(5-methylfuran-3-yl)isoxazolidine-2-carbonyl)cyclohexyl)methyl)-2H-indazole-6-carbonitrile (Compound (41)) is performed as in STEP 3c here-above but starting from (3S)-3-(5-methylfuran-3-yl)isoxazolidine instead of 3-fluoro-5-[(3S)-isoxazolidin-3-yl]benzonitrile hydrochloride.

STEP 3f: Synthesis of trans-1-((4-((S)-3-(5-methylfuran-3-yl)isoxazolidine-2-carbonyl)cyclohexyl)methyl)-1H-indazole-6-carbonitrile (Compound (43)) is performed as in STEP 3a here-above but starting from (3S)-3-(5-methylfuran-3-yl)isoxazolidine instead of 3-fluoro-5-[(3S)-isoxazolidin-3-yl]benzonitrile hydrochloride.

STEP 3g: Synthesis of trans-2-((4-((S)-3-(5-cyanopyridin-3-yl)isoxazolidine-2-carbonyl)cyclohexyl)methyl)-2H-indazole-6-carbonitrile (Compound (127)) is performed as in STEP 3a here-above but starting from (3S)-3-(5-cyanopyridin-3-yl)isoxazolidine instead of 3-fluoro-5-[(3S)-isoxazolidin-3-yl]benzonitrile hydrochloride.

STEP 3h: trans-2-[[4-[(3S)-3-(6-methoxypyrazin-2-yl)isoxazolidine-2-carbonyl]cyclohexyl]methyl]indazole-6-carbonitrile (Compound (189)) is performed as in STEP 3a here-above but starting from (3S)-3-(6-methoxypyrazin-2-yl)isoxazolidine instead of 3-fluoro-5-[(3S)-isoxazolidin-3-yl]benzonitrile hydrochloride.

STEP 3j: Synthesis of trans-1-[[4-[(3S)-3-(6-methoxypyrazin-2-yl)isoxazolidine-2-carbonyl]cyclohexyl]methyl]indazole-6-carbonitrile (Compound (190)) is performed as in STEP 3a here-above but starting from (3S)-3-(6-methoxypyrazin-2-yl)isoxazolidine instead of 3-fluoro-5-[(3S)-isoxazolidin-3-yl]benzonitrile hydrochloride.

STEP 3k: Synthesis of trans-2-[[4-[(3S)-3-pyrazin-2-ylisoxazolidine-2-carbonyl]cyclohexyl]methyl]indazole-6-carbonitrile (Compound (196)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3l: Synthesis of trans-1-[[4-[(3S)-3-pyrazin-2-ylisoxazolidine-2-carbonyl]cyclohexyl]methyl]indazole-6-carbonitrile (Compound (200)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3m: Synthesis of trans-2-[[4-[(3S)-3-(2-methylthiazol-4-yl)isoxazolidine-2-carbonyl]cyclohexyl]methyl]indazole-6-carbonitrile (Compound (203)) is performed as in STEP 3a here-above but starting from (3S)-3-(2-methylthiazol-4-yl)isoxazolidine instead of 3-fluoro-5-[(3S)-isoxazolidin-3-yl]benzonitrile hydrochloride.

STEP 3n: Synthesis of trans-1-[[4-[(3S)-3-(2-methylthiazol-4-yl)isoxazolidine-2-carbonyl]cyclohexyl]methyl]indazole-6-carbonitrile (Compound (204)) is performed as in STEP 3a here-above but starting from (3S)-3-(2-methylthiazol-4-yl)isoxazolidine instead of 3-fluoro-5-[(3S)-isoxazolidin-3-yl]benzonitrile hydrochloride.

STEP 3o: Synthesis of trans-1-[[4-[(3S)-3-(2-pyridyl)isoxazolidine-2-carbonyl]cyclohexyl]methyl]indazole-6-carbonitrile (Compound (215)) is performed as in STEP 3a here-above but starting from (3S)-3-(2-pyridyl)isoxazolidine instead of 3-fluoro-5-[(3S)-isoxazolidin-3-yl]benzonitrile hydrochloride.

### Example 3.2: Synthesis of Compounds (32), (33), (44)

STEP 1: Synthesis of methyl trans-4-[(6-cyano-4-fluoro-indazol-1-yl)methyl]cyclohexanecarboxylate—heteroaromatic compound containing a Nitrogen atom used in step 6a of Scheme 3 in a similar way as performed in example 3.1 step 1, but starting from 6-cyano-4-fluoro-1H-indazole.

STEP 2: Synthesis of trans-4-[(6-cyano-4-fluoro-indazol-1-yl)methyl]cyclohexanecarboxylic acid—Compound (Iia) in Scheme 1—in a similar way as performed in Example 1.1, but using methyl trans-4-[(6-cyano-4-fluoro-indazol-1-yl)methyl]cyclohexanecarboxylate instead of trans-4-(benzimidazol-1-ylmethyl)cyclohexanecarboxylate

STEP 3a: Synthesis of trans-1-((4-((S)-3-(3-cyano-5-fluorophenyl)isoxazolidine-2-carbonyl)cyclohexyl)methyl)-4-fluoro-1H-indazole-6-carbonitrile (Compound (32)) is performed as in STEP 5c of Example 1.4, but starting from trans-4-[(6-cyano-4-fluoro-indazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-4-fluoro-1H-indazole-6-carbonitrile (Compound (33)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3c: Synthesis of trans-4-fluoro-1-((4-((S)-3-(5-methylfuran-3-yl)isoxazolidine-2-carbonyl)cyclohexyl)methyl)-1H-indazole-6-carbonitrile (Compound (44)) is performed as in STEP 3a here-above but starting from (3S)-3-(5-methylfuran-3-yl)isoxazolidine instead of 3-fluoro-5-[(3S)-isoxazolidin-3-yl]benzonitrile hydrochloride.

### Example 3.3: Synthesis of Compounds (34), (35), (42)

STEP 1: Synthesis of methyl trans-4-[(6-cyano-4-fluoro-indazol-2-yl)methyl]cyclohexanecarboxylate—heteroaromatic compound containing a Nitrogen atom used in step 6a of Scheme 3 in a similar way as performed in example 3.1 step 1, but starting from 6-cyano-4-fluoro-1H-indazole

STEP 2: Synthesis of trans-4-[(6-cyano-4-fluoro-indazol-2-yl)methyl]cyclohexanecarboxylic acid—Compound (Iia) in Scheme 1—in a similar way as performed in Example 1.1, but using methyl trans-4-[(6-cyano-4-fluoro-indazol-2-yl)methyl]cyclohexanecarboxylate instead of trans-4-(benzimidazol-1-ylmethyl)cyclohexanecarboxylate

STEP 3a: Synthesis of trans-2-((4-((S)-3-(3-cyano-5-fluorophenyl)isoxazolidine-2-carbonyl)cyclohexyl)methyl)-4-fluoro-2H-indazole-6-carbonitrile (Compound (34)) is performed as in STEP 5c of Example 1.4, but starting from trans-4-[(6-cyano-4-fluoro-indazol-2-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-2-((4-((S)-3-(3,5-difluorophenyl)isoxazolidine-2-carbonyl)cyclohexyl)methyl)-4-fluoro-2H-indazole-6-carbonitrile (Compound (35)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3c: Synthesis of trans-4-fluoro-2-((4-((S)-3-(5-methylfuran-3-yl)isoxazolidine-2-carbonyl)cyclohexyl)methyl)-2H-indazole-6-carbonitrile (Compound (42)) is performed as in STEP 3a here-above but starting from (3S)-3-(5-methylfuran-3-yl)isoxazolidine instead of 3-fluoro-5-[(3S)-isoxazolidin-3-yl]benzonitrile hydrochloride.

### Example 3.4: Synthesis of Compounds (45), (46), (47), (130)

STEP 1: Synthesis of methyl trans-4-[(6-cyano-4-fluoro-indol-1-yl)methyl]cyclohexanecarboxylate—heteroaromatic compound containing a Nitrogen atom used in step 6a of Scheme 3 in a similar way as performed in example 3.1 step 1, but starting from 6-cyano-4-fluoro-indole

STEP 2: Synthesis of trans-4-[(6-cyano-4-fluoro-indol-1-yl)methyl]cyclohexanecarboxylic acid—Compound (Iia) in Scheme 1—in a similar way as performed in Example 1.1, but using methyl trans-4-[(6-cyano-4-fluoro-indol-1-yl)methyl]cyclohexanecarboxylate instead of trans-4-(benzimidazol-1-ylmethyl)cyclohexanecarboxylate

STEP 3a: Synthesis of trans-1-((4-((S)-3-(5-cyanopyridin-3-yl)isoxazolidine-2-carbonyl)cyclohexyl)methyl)-4-fluoro-1H-indole-6-carbonitrile (Compound (130)) is performed as in STEP 5a of Example 1.3, but starting from trans-4-[(6-cyano-4-fluoro-indol-1-yl)methyl]cyclohexanecarboxylic acid instead of trans-4-[(6-cyano-5-fluoro-benzimidazol-1-yl)methyl]cyclohexanecarboxylic acid.

STEP 3b: Synthesis of trans-1-((4-((S)-3-(3-cyano-5-fluorophenyl)isoxazolidine-2-carbonyl)cyclohexyl)methyl)-4-fluoro-1H-indole-6-carbonitrile (Compound (46)) is performed as in STEP 5c of Example 1.4, but starting from trans-4-[(6-cyano-4-fluoro-indol-1-yl)methyl]cyclohexanecarboxylic acid instead of trans-4-[(5-cyano-6-fluoro-benzimidazol-1-yl)methyl]cyclohexanecarboxylic acid.

STEP 3c: Synthesis of trans-1-((4-((S)-3-(3,5-difluorophenyl)isoxazolidine-2-carbonyl)cyclohexyl)methyl)-4-fluoro-1H-indole-6-carbonitrile (Compound (45)) is performed as in STEP 3b here-above but starting from (3S)-3-(3,5-difluorophenyl)isoxazolidine instead of 3-fluoro-5-[(3S)-isoxazolidin-3-yl]benzonitrile hydrochloride.

STEP 3d: Synthesis of trans-4-fluoro-1-((4-((S)-3-(5-methylfuran-3-yl)isoxazolidine-2-carbonyl)cyclohexyl)methyl)-1H-indole-6-carbonitrile (Compound (47)) is performed as in STEP 3b here-above but starting from (3S)-3-(5-methylfuran-3-yl)isoxazolidine instead of 3-fluoro-5-[(3S)-isoxazolidin-3-yl]benzonitrile hydrochloride.

### Example 3.5: Synthesis of Compounds (52), (53), (202), (217), (218)

STEP 1: Synthesis of methyl trans-4-[(5-cyanoindazol-1-yl)methyl]cyclohexanecarboxylate—heteroaromatic compound containing a Nitrogen atom used in step 6a of Scheme 3 in a similar way as performed in example 3.1 step 1, but starting from 5-cyano-1H-indazole

STEP 2: Synthesis of trans-4-[(5-cyanoindazol-1-yl)methyl]cyclohexanecarboxylic acid—Compound (Iia) in Scheme 1—in a similar way as performed in Example 1.1, but using methyl trans-4-[(5-cyanoindazol-1-yl)methyl]cyclohexanecarboxylate instead of trans-4-(benzimidazol-1-ylmethyl)cyclohexanecarboxylate

STEP 3a: Synthesis of trans-1-[[4-[(3S)-3-(2-methylthiazol-4-yl)isoxazolidine-2-carbonyl]cyclohexyl]methyl]indazole-5-carbonitrile (Compound (202)) is performed as in STEP 5a of Example 1.3, but starting from trans-4-[(5-cyanoindazol-1-yl)methyl]cyclohexanecarboxylic acid and (3S)-3-(2-methylthiazol-4-yl)isoxazolidine instead of trans-4-[(6-cyano-5-fluoro-benzimidazol-1-yl)methyl]cyclohexanecarboxylic acid and 5-[(3S)-isoxazolidin-3-yl]pyridine-3-carbonitrile respectively.

STEP 3b: Synthesis of trans-1-[[4-[(3S)-3-(6-methoxypyrazin-2-yl)isoxazolidine-2-carbonyl]cyclohexyl]methyl]indazole-5-carbonitrile (Compound (217)) is performed as in STEP 3a here-above, but starting from (3S)-3-(6-methoxypyrazin-2-yl)isoxazolidine instead of (3S)-3-(2-methylthiazol-4-yl)isoxazolidine.

STEP 3c: Synthesis of trans-1-[[4-[(3S)-3-pyrazin-2-ylisoxazolidine-2-carbonyl]cyclohexyl]methyl]indazole-5-carbonitrile (Compound (218)) is performed as in STEP 3a here-above, but starting from (3S)-3-pyrazin-2-ylisoxazolidine instead of (3S)-3-(2-methylthiazol-4-yl)isoxazolidine.

STEP 3d: Synthesis of trans-1-((4-((S)-3-(3-cyano-5-fluorophenyl)isoxazolidine-2-carbonyl)cyclohexyl)methyl)-1H-indazole-5-carbonitrile (Compound (53)) is performed as in STEP 5c of Example 1.4, but starting from trans-4-[(5-cyanoindazol-1-yl)methyl]cyclohexanecarboxylic acid instead of trans-4-[(5-cyano-6-fluoro-benzimidazol-1-yl)methyl]cyclohexanecarboxylic acid.

STEP 3e: Synthesis of trans-1-((4-((S)-3-(3,5-difluorophenyl)isoxazolidine-2-carbonyl)cyclohexyl)methyl)-1H-indazole-5-carbonitrile (Compound (52)) is performed as in STEP 3d here-above but starting from (3S)-3-(3,5-difluorophenyl)isoxazolidine instead of 3-fluoro-5-[(3S)-isoxazolidin-3-yl]benzonitrile hydrochloride.

### Example 3.6: Synthesis of Compounds (54), (55), (126)

STEP 1: Synthesis of methyl trans-4-[(5-fluoroindazol-1-yl)methyl]cyclohexanecarboxylate—heteroaromatic compound containing a Nitrogen atom used in step 6a of Scheme 3 in a similar way as performed in example 3.1 step 1, but starting from 5-fluoro-1H-indazole

STEP 2: Synthesis of trans-4-[(5-fluoroindazol-1-yl)methyl]cyclohexanecarboxylic acid—Compound (Iia) in Scheme 1—in a similar way as performed in Example 1.1, but using methyl trans-4-[(5-fluoroindazol-1-yl)methyl]cyclohexanecarboxylate instead of trans-4-(benzimidazol-1-ylmethyl)cyclohexanecarboxylate

STEP 3a: Synthesis of trans-5-((S)-2-(4-((5-fluoro-1H-indazol-1-yl)methyl)cyclohexane-1-carbonyl)isoxazolidin-3-yl)nicotinonitrile (Compound (126)) is performed as in STEP 5a of Example 1.3, but starting from trans-4-[(5-fluoroindazol-1-yl)methyl]cyclohexanecarboxylic acid instead of trans-4-[(6-cyano-5-fluoro-benzimidazol-1-yl)methyl]cyclohexanecarboxylic acid.

STEP 3b: Synthesis of trans-3-fluoro-5-((S)-2-(4-((5-fluoro-1H-indazol-1-yl)methyl)cyclohexane-1-carbonyl)isoxazolidin-3-yl)benzonitrile (Compound (54)) is performed as in STEP 5c of Example 1.4, but starting from trans-4-[(5-fluoroindazol-1-yl)methyl]cyclohexanecarboxylic acid instead of trans-4-[(5-cyano-6-fluoro-benzimidazol-1-yl)methyl]cyclohexanecarboxylic acid.

STEP 3c: Synthesis of trans-((S)-3-(3,5-difluorophenyl)isoxazolidin-2-yl)(4-((5-fluoro-1H-indazol-1-yl)methyl)cyclohexyl)methanone (Compound (55)) is performed as in STEP 3b here-above but starting from (3S)-3-(3,5-difluorophenyl)isoxazolidine instead of 3-fluoro-5-[(3S)-isoxazolidin-3-yl]benzonitrile hydrochloride.

### Example 3.7: Synthesis of Compounds (56), (57)

STEP 1: Synthesis of methyl trans-4-[(5-fluoro indazol-2-yl)methyl]cyclohexanecarboxylate—heteroaromatic compound containing a Nitrogen atom used in step 6a of Scheme 3 in a similar way as performed in example 3.1 step 1, but starting from 5-fluoro-1H-indazole

STEP 2: Synthesis of trans-4-[(5-fluoroindazol-2-yl)methyl]cyclohexanecarboxylic acid—Compound (Iia) in Scheme 1—in a similar way as performed in Example 1.1, but using methyl trans-4-[(5-fluoro indazol-2-yl)methyl]cyclohexanecarboxylate instead of trans-4-(benzimidazol-1-ylmethyl)cyclohexanecarboxylate

STEP 3a: Synthesis of trans-3-fluoro-5-((S)-2-(4-((5-fluoro-2H-indazol-2-yl)methyl)cyclohexane-1-carbonyl)isoxazolidin-3-yl)benzonitrile (Compound (56)) is performed as in STEP 5c of Example 1.4, but starting from trans-4-[(5-fluoroindazol-2-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-((S)-3-(3,5-difluorophenyl)isoxazolidin-2-yl)(4-((5-fluoro-2H-indazol-2-yl)methyl)cyclohexyl)methanone (Compound (57)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.8: Synthesis of Compounds (58), (59)

STEP 1: Synthesis of methyl trans-4-[(5-cyanoindazol-2-yl)methyl]cyclohexanecarboxylate—heteroaromatic compound containing a Nitrogen atom used in step 6a of Scheme 3 in a similar way as performed in example 3.1 step 1, but starting from 5-cyano-1H-indazole

STEP 2: Synthesis of trans-4-[(5-cyanoindazol-2-yl)methyl]cyclohexanecarboxylic acid—Compound (Iia) in Scheme 1—in a similar way as performed in Example 1.1, but using methyl trans-4-[(5-cyanoindazol-2-yl)methyl]cyclohexanecarboxylate instead of trans-4-(benzimidazol-1-ylmethyl)cyclohexanecarboxylate

STEP 3a: Synthesis of trans-2-((4-((S)-3-(3-cyano-5-fluorophenyl)isoxazolidine-2-carbonyl)cyclohexyl)methyl)-2H-indazole-5-carbonitrile (Compound (59)) is performed as in STEP 5c of Example 1.4, but starting from trans-4-[(5-cyanoindazol-2-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-2-((4-((S)-3-(3,5-difluorophenyl)isoxazolidine-2-carbonyl)cyclohexyl)methyl)-2H-indazole-5-carbonitrile (Compound (58)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.9: Synthesis of Compounds (60), (61)

STEP 1: Synthesis of methyl trans-4-[(3-methylindazol-1-yl)methyl]cyclohexanecarboxylate—heteroaromatic compound containing a Nitrogen atom used in step 6a of Scheme 3 in a similar way as performed in example 3.1 step 1, but starting from 3-methyl-1H-indazole STEP 2: Synthesis of trans-4-[(3-methylindazol-1-yl)methyl]cyclohexanecarboxylic acid—Compound (Iia) in Scheme 1—in a similar way as performed in Example 1.1, but using methyl trans-4-[(3-methylindazol-1-yl)methyl]cyclohexanecarboxylate instead of trans-4-(benzimidazol-1-ylmethyl)cyclohexanecarboxylate

STEP 3a: Synthesis of trans-3-fluoro-5-((S)-2-(4-((3-methyl-1H-indazol-1-yl)methyl)cyclohexane-1-carbonyl)isoxazolidin-3-yl)benzonitrile (Compound (61)) is performed as in STEP 5c of Example 1.4, but starting from trans-4-[(3-methylindazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-((S)-3-(3,5-difluorophenyl)isoxazolidin-2-yl)(4-((3-methyl-1H-indazol-1-yl)methyl)cyclohexyl)methanone (Compound (60)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.10: Synthesis of Compounds (62), (63)

STEP 1: Synthesis of methyl trans-4-[(3-methylindazol-2-yl)methyl]cyclohexanecarboxylate—heteroaromatic compound containing a Nitrogen atom used in step 6a of Scheme 3 in a similar way as performed in example 3.1 step 1, but starting from 3-methyl-1H-indazole

STEP 2: Synthesis of trans-4-[(3-methylindazol-2-yl)methyl]cyclohexanecarboxylic acid—Compound (Iia) in Scheme 1—in a similar way as performed in Example 1.1, but using methyl trans-4-[(3-methylindazol-2-yl)methyl]cyclohexanecarboxylate instead of trans-4-(benzimidazol-1-ylmethyl)cyclohexanecarboxylate

STEP 3a: Synthesis of trans-3-fluoro-5-((S)-2-(4-((3-methyl-2H-indazol-2-yl)methyl)cyclohexane-1-carbonyl)isoxazolidin-3-yl)benzonitrile (Compound (63)) is performed as in STEP 5c of Example 1.4, but starting from trans-4-[(3-methylindazol-2-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-((S)-3-(3,5-difluorophenyl)isoxazolidin-2-yl)(4-((3-methyl-2H-indazol-2-yl)methyl)cyclohexyl)methanone (Compound (62)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.11: Synthesis of Compounds (82), (83)

STEP 1: Synthesis of methyl trans-4-[(4,5-dimethylimidazol-1-yl)methyl]cyclohexanecarboxylate—heteroaromatic compound containing a Nitrogen atom used in step 6a of Scheme 3 in a similar way as performed in example 3.1 step 1, but starting from 4,5-dimethylimidazole

STEP 2: Synthesis of trans-4-[(4,5-dimethylimidazol-1-yl)methyl]cyclohexanecarboxylic acid—Compound (Iia) in Scheme 1—in a similar way as performed in Example 1.1, but using methyl trans-4-[(4,5-dimethylimidazol-1-yl)methyl]cyclohexanecarboxylate instead of trans-4-(benzimidazol-1-ylmethyl)cyclohexanecarboxylate

STEP 3a: Synthesis of trans-3-((S)-2-(4-((4,5-dimethyl-1H-imidazol-1-yl)methyl)cyclohexane-1-carbonyl)isoxazolidin-3-yl)-5-fluorobenzonitrile (Compound (82)) is performed as in STEP 5c of Example 1.4, but starting from trans-4-[(4,5-dimethylimidazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-((S)-3-(3,5-difluorophenyl)isoxazolidin-2-yl)(4-((4,5-dimethyl-1H-imidazol-1-yl)methyl)cyclohexyl)methanone (Compound (83)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.12: Synthesis of Compounds (88), (89)

STEP 1: Synthesis of methyl trans-4-[(5-cyano-4-methyl-imidazol-1-yl)methyl]cyclohexanecarboxylate—heteroaromatic compound containing a Nitrogen atom used in step 6a of Scheme 3 in a similar way as performed in example 3.1 step 1, but starting from 5-cyano-4-methyl-imidazole

STEP 2: Synthesis of trans-4-[(5-cyano-4-methyl-imidazol-1-yl)methyl]cyclohexanecarboxylic acid—Compound (Iia) in Scheme 1—in a similar way as performed in Example 1.1, but using methyl trans-4-[(5-cyano-4-methyl-imidazol-1-yl)methyl]cyclohexanecarboxylate instead of trans-4-(benzimidazol-1-ylmethyl)cyclohexanecarboxylate

STEP 3a: Synthesis of trans-1-((4-((S)-3-(3-cyano-5-fluorophenyl)isoxazolidine-2-carbonyl)cyclohexyl)methyl)-4-methyl-1H-imidazole-5-carbonitrile (Compound (88)) is performed as in STEP 5c of Example 1.4, but starting from trans-4-[(5-cyano-4-methyl-imidazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-4-methyl-1H-imidazole-5-carbonitrile (Compound (89)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.13: Synthesis of Compounds (90), (91)

STEP 1: Synthesis of methyl trans-4-[(4-cyano-5-methyl-imidazol-1-yl)methyl]cyclohexanecarboxylate—heteroaromatic compound containing a Nitrogen atom used in step 6a of Scheme 3 in a similar way as performed in example 3.1 step 1, but starting from 4-cyano-5-methyl-imidazole

STEP 2: Synthesis of trans-4-[(4-cyano-5-methyl-imidazol-1-yl)methyl]cyclohexanecarboxylic acid—Compound (Iia) in Scheme 1—in a similar way as performed in Example 1.1, but using methyl trans-4-[(4-cyano-5-methyl-imidazol-1-yl)methyl]cyclohexanecarboxylate instead of trans-4-(benzimidazol-1-ylmethyl)cyclohexanecarboxylate

STEP 3a: Synthesis of trans-1-((4-((S)-3-(3-cyano-5-fluorophenyl)isoxazolidine-2-carbonyl)cyclohexyl)methyl)-5-methyl-1H-imidazole-4-carbonitrile (Compound (90)) is performed as in STEP 5c of Example 1.4, but starting from trans-4-[(4-cyano-5-methyl-imidazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-5-methyl-1H-imidazole-4-carbonitrile (Compound (91)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.14: Synthesis of Compounds (93), (94), (123)

STEP 1: Synthesis of methyl trans-4-[(6-fluoropyrrolo[3,2-b]pyridin-1-yl)methyl]cyclohexanecarboxylate—heteroaromatic compound containing a Nitrogen atom used in step 6a of Scheme 3 in a similar way as performed in example 3.1 step 1, but starting from 6-fluoropyrrolo[3,2-b]pyridine

STEP 2: Synthesis of trans-4-[(6-fluoropyrrolo[3,2-b]pyridin-1-yl)methyl]cyclohexane carboxylic acid—Compound (Iia) in Scheme 1—in a similar way as performed in Example 1.1, but using methyl trans-4-[(6-fluoropyrrolo[3,2-b]pyridin-1-yl)methyl]cyclohexanecarboxylate instead of trans-4-(benzimidazol-1-ylmethyl)cyclohexanecarboxylate

STEP 3a: Synthesis of trans-5-((S)-2-(4-((6-fluoro-1H-pyrrolo[3,2-b]pyridin-1-yl)methyl)cyclohexane-1-carbonyl)isoxazolidin-3-yl)nicotinonitrile (Compound (123)) is performed as in STEP 5a of Example 1.3, but starting from trans-4-[(6-fluoropyrrolo[3,2-b]pyridin-1-yl)methyl]cyclohexane carboxylic acid instead of trans-4-[(6-cyano-5-fluoro-benzimidazol-1-yl)methyl]cyclohexanecarboxylic acid.

STEP 3b: Synthesis of trans-3-fluoro-5-((S)-2-(4-((6-fluoro-1H-pyrrolo[3,2-b]pyridin-1-yl)methyl)cyclohexane-1-carbonyl)isoxazolidin-3-yl)benzonitrile (Compound (93)) is performed as in STEP 5c of Example 1.4, but starting from trans-4-[(6-fluoropyrrolo[3,2-b]pyridin-1-yl)methyl]cyclohexane carboxylic acid instead of trans-4-[(5-cyano-6-fluoro-benzimidazol-1-yl)methyl]cyclohexanecarboxylic acid.

STEP 3c: Synthesis of trans-((S)-3-(3,5-difluorophenyl)isoxazolidin-2-yl)(4-((6-fluoro-1H-pyrrolo[3,2-b]pyridin-1-yl)methyl)cyclohexyl)methanone (Compound (94)) is performed as in STEP 3b here-above but starting from (3S)-3-(3,5-difluorophenyl)isoxazolidine instead of 3-fluoro-5-[(3S)-isoxazolidin-3-yl]benzonitrile hydrochloride.

### Example 3.15: Synthesis of Compounds (95), (96)

STEP 1: Synthesis of methyl trans-4-[(5-fluoroindol-1-yl)methyl]cyclohexanecarboxylate—heteroaromatic compound containing a Nitrogen atom used in step 6a of Scheme 3 in a similar way as performed in example 3.1 step 1, but starting from 5-fluoroindole

STEP 2: Synthesis of trans-4-[(5-fluoroindol-1-yl)methyl]cyclohexanecarboxylic acid—Compound (Iia) in Scheme 1—in a similar way as performed in Example 1.1, but using methyl trans-4-[(5-fluoroindol-1-yl)methyl]cyclohexanecarboxylate instead of trans-4-(benzimidazol-1-ylmethyl)cyclohexanecarboxylate

STEP 3a: Synthesis of trans-3-fluoro-5-((S)-2-(4-((5-fluoro-1H-indol-1-yl)methyl)cyclohexane-1-carbonyl)isoxazolidin-3-yl)benzonitrile (Compound (95)) is performed as in STEP 5c of Example 1.4, but starting from trans-4-[(5-fluoroindol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-((S)-3-(3,5-difluorophenyl)isoxazolidin-2-yl)(4-((5-fluoro-1H-indol-1-yl)methyl)cyclohexyl)methanone (Compound (96)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.16: Synthesis of Compounds (104), (105)

STEP 1: Synthesis of methyl trans-4-[(6-carbamoyl indol-1-yl)methyl]cyclohexanecarboxylate—heteroaromatic compound containing a Nitrogen atom used in step 6a of Scheme 3 in a similar way as performed in example 3.1 step 1, but starting from 6-carbamoylindole

STEP 2: Synthesis of the corresponding carboxylic acid from methyl trans-4-[(6-carbamoyl indol-1-yl)methyl]cyclohexanecarboxylate acid—Compound (Iia) in Scheme 1—in a similar way as performed in Example 1.1

STEP 3a: Synthesis of trans-1-((4-((S)-3-(3-cyano-5-fluorophenyl)isoxazolidine-2-carbonyl)cyclohexyl)methyl)-1H-indole-6-carboxamide (Compound (104)) is performed as in STEP 5c of Example 1.4, but starting from trans-4-[(6-carbamoyl indol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-1H-indole-6-carboxamide (Compound (105)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.17: Synthesis of Compounds (2), (3), (17), (108), (134), (185), (188), (198)

STEP 1: Synthesis of methyl trans-4-[(6-cyanopyrrolo [3,2-b]pyridin-1-yl)methyl]cyclohexanecarboxylate—heteroaromatic compound containing a Nitrogen atom used in step 6a of Scheme 3 in a similar way as performed in example 3.1 step 1, but starting from 6-cyanopyrrolo [3,2-b]pyridine

STEP 2: Synthesis of trans-4-[(6-cyanopyrrolo[3,2-b]pyridin-1-yl)methyl]cyclohexanecarboxylic acid—Compound (Iia) in Scheme 1—in a similar way as performed in Example 1.1, but using methyl trans-4-[(6-cyanopyrrolo [3,2-b]pyridin-1-yl)methyl]cyclohexanecarboxylate instead of trans-4-(benzimidazol-1-ylmethyl)cyclohexanecarboxylate.

STEP 3a: Synthesis of trans-1-((4-((S)-3-(3-cyano-5-fluorophenyl)isoxazolidine-2-carbonyl)cyclohexyl)methyl)-1H-pyrrolo[3,2-b]pyridine-6-carbonitrile (Compound (2)) is performed as in STEP 5c of Example 1.4, but starting from trans-4-[(6-cyanopyrrolo[3,2-b]pyridin-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-1-((4-((S)-3-(5-fluoropyridin-3-yl)isoxazolidine-2-carbonyl)cyclohexyl)methyl)-1H-pyrrolo[3,2-b]pyridine-6-carbonitrile (Compound (3)) is performed as in STEP 3a here-above but starting from (3S)-3-(5-fluoropyridin-3-yl)isoxazolidine instead of 3-fluoro-5-[(3S)-isoxazolidin-3-yl]benzonitrile hydrochloride.

STEP 3c: Synthesis of trans-1-((4-((S)-3-(3,5-difluorophenyl)isoxazolidine-2-carbonyl)cyclohexyl)methyl)-1H-pyrrolo[3,2-b]pyridine-6-carbonitrile (Compound (17)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3d: Synthesis of trans-1-((4-((S)-3-(5-cyanopyridin-3-yl)isoxazolidine-2-carbonyl)cyclohexyl)methyl)-1H-pyrrolo[3,2-b]pyridine-6-carbonitrile (Compound (108)) is performed as in STEP 3a here-above but starting from (3S)-3-(5-cyanopyridin-3-yl)isoxazolidine instead of 3-fluoro-5-[(3S)-isoxazolidin-3-yl]benzonitrile hydrochloride.

STEP 3e: Synthesis of trans-1-[[4-[(3S)-3-pyrazin-2-ylisoxazolidine-2-carbonyl]cyclohexyl]methyl]pyrrolo[3,2-b]pyridine-6-carbonitrile (Compound (134)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3f: Synthesis of trans-1-[[4-[(3S)-3-(6-methoxypyrazin-2-yl)isoxazolidine-2-carbonyl]cyclohexyl]methyl]pyrrolo[3,2-b]pyridine-6-carbonitrile (Compound (185)) is performed as in STEP 3a here-above but starting from (3S)-3-(6-methoxypyrazin-2-yl)isoxazolidine instead of 3-fluoro-5-[(3S)-isoxazolidin-3-yl]benzonitrile hydrochloride.

STEP 3g: Synthesis of trans-1-[[4-[(3S)-3-(2-pyridyl)isoxazolidine-2-carbonyl]cyclohexyl]methyl]pyrrolo[3,2-b]pyridine-6-carbonitrile (Compound (188)) is performed as in STEP 3a here-above but starting from (3S)-3-(2-pyridyl)isoxazolidine instead of 3-fluoro-5-[(3S)-isoxazolidin-3-yl]benzonitrile hydrochloride.

STEP 3h: Synthesis of trans-1-[[4-[(3S)-3-(2-methylthiazol-4-yl)isoxazolidine-2-carbonyl]cyclohexyl]methyl]pyrrolo[3,2-b]pyridine-6-carbonitrile (Compound (198)) is performed as in STEP 3a here-above but starting from (3S)-3-(2-methylthiazol-4-yl)isoxazolidine instead of 3-fluoro-5-[(3S)-isoxazolidin-3-yl]benzonitrile hydrochloride.

### Example 3.18: Synthesis of Compounds (110), (111)

STEP 1: Synthesis of methyl trans-4-[(6-carbamoyl-3-methyl-indol-1-yl)methyl]cyclohexanecarboxylate—heteroaromatic compound containing a Nitrogen atom used in step 6a of Scheme 3 in a similar way as performed in example 3.1 step 1, but starting from 6-carbamoyl-3-methyl-indole.

STEP 2: Synthesis of trans-4-[(6-carbamoyl-3-methyl-indol-1-yl)methyl]cyclohexane carboxylic acid—Compound (Iia) in Scheme 1—in a similar way as performed in Example 1.1, but using methyl trans-4-[(6-carbamoyl-3-methyl-indol-1-yl)methyl]cyclohexanecarboxylate instead of trans-4-(benzimidazol-1-ylmethyl)cyclohexanecarboxylate

STEP 3a: Synthesis of trans-1-((4-((S)-3-(3-cyano-5-fluorophenyl)isoxazolidine-2-carbonyl)cyclohexyl)methyl)-3-methyl-1H-indole-6-carboxamide (Compound (110)) is performed as in STEP 5c of Example 1.4, but starting from trans-4-[(6-carbamoyl-3-methyl-indol-1-yl)methyl]cyclohexane carboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-3-methyl-1H-indole-6-carboxamide (Compound (111)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.19: Synthesis of Compounds (117), (118), (119)

STEP 1: Synthesis of methyl trans-4-[(3,5-dimethyl pyrazol-1-yl)methyl]cyclohexanecarboxylate—heteroaromatic compound containing a Nitrogen atom used in step 6a of Scheme 3 in a similar way as performed in example 3.1 step 1, but starting from 3,5-dimethyl pyrazole

STEP 2: Synthesis of trans-4-[(3,5-dimethylpyrazol-1-yl)methyl]cyclohexanecarboxylic acid—Compound (Iia) in Scheme 1—in a similar way as performed in Example 1.1, but using methyl trans-4-[(3,5-dimethyl pyrazol-1-yl)methyl]cyclohexanecarboxylate instead of trans-4-(benzimidazol-1-ylmethyl)cyclohexanecarboxylate

STEP 3a: Synthesis of trans-3-((S)-2-(4-((3,5-dimethyl-1H-pyrazol-1-yl)methyl)cyclohexane-1-carbonyl)isoxazolidin-3-yl)-5-fluorobenzonitrile (Compound (119)) is performed as in STEP 5c of Example 1.4, but starting from trans-4-[(3,5-dimethylpyrazol-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-(4-((3,5-dimethyl-1H-pyrazol-1-yl)methyl)cyclohexyl)((S)-3-(p-tolyl)isoxazolidin-2-yl)methanone (Compound (117)) is performed as in STEP 3a here-above but starting from (3S)-3-(p-tolyl)isoxazolidine instead of 3-fluoro-5-[(3S)-isoxazolidin-3-yl]benzonitrile hydrochloride.

STEP 3c: Synthesis of trans-((S)-3-(3,5-difluorophenyl)isoxazolidin-2-yl)(4-((3,5-dimethyl-1H-pyrazol-1-yl)methyl)cyclohexyl)methanone (Compound (118)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.20: Synthesis of Compounds (26), (27), (121), (137), (186), (187), (199)

STEP 1: Synthesis of methyl trans-4-[(6-cyanopyrazolo[4,3-b]pyridin-1-yl)methyl]cyclohexanecarboxylate—heteroaromatic compound containing a Nitrogen atom used in step 6a of Scheme 3 in a similar way as performed in example 3.1 step 1, but starting from 6-cyanopyrazolo[4,3-b]pyridine

STEP 2: Synthesis of trans-4-[(6-cyanopyrazolo[4,3-b]pyridin-1-yl)methyl]cyclohexane carboxylic acid—Compound (Iia) in Scheme 1—in a similar way as performed in Example 1.1, but using methyl trans-4-[(6-cyanopyrazolo[4,3-b]pyridin-1-yl)methyl]cyclohexanecarboxylate instead of trans-4-(benzimidazol-1-ylmethyl)cyclohexanecarboxylate

STEP 3a: Synthesis of trans-1-((4-((S)-3-(3-cyano-5-fluorophenyl)isoxazolidine-2-carbonyl)cyclohexyl)methyl)-1H-pyrazolo[4,3-b]pyridine-6-carbonitrile (Compound (27)) is performed as in STEP 5c of Example 1.4, but starting from trans-4-[(6-cyanopyrazolo[4,3-b]pyridin-1-yl)methyl]cyclohexane carboxylic acid instead of trans-4-[(5-cyano-6-fluoro-benzimidazol-1-yl)methyl]cyclohexanecarboxylic acid.

STEP 3b: Synthesis of trans-1-((4-((S)-3-(3,5-difluorophenyl)isoxazolidine-2-carbonyl)cyclohexyl)methyl)-1H-pyrazolo[4,3-b]pyridine-6-carbonitrile (Compound (26)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3c: Synthesis of trans-1-[[4-[(3S)-3-pyrazin-2-ylisoxazolidine-2-carbonyl]cyclohexyl]methyl]pyrazolo[4,3-b]pyridine-6-carbonitrile (Compound (137)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3d: Synthesis of trans-2-((4-((S)-3-(5-cyanopyridin-3-yl)isoxazolidine-2-carbonyl)cyclohexyl)methyl)-2H-pyrazolo[3,4-b]pyridine-5-carbonitrile (Compound (121)) is performed as in STEP 3a here-above but starting from trans-4-[(5-cyanopyrazolo[4,3-b]pyridin-1-yl)methyl]cyclohexane carboxylic acid and (S)-3-(5-cyanopyridin-3-yl)isoxazolidine instead of trans-4-[(6-cyanopyrazolo[4,3-b]pyridin-1-yl)methyl]cyclohexane carboxylicacid and 3-fluoro-5-[(3S)-isoxazolidin-3-yl]benzonitrile hydrochloride.

STEP 3e: Synthesis of trans-1-[[4-[(3S)-3-(6-methoxypyrazin-2-yl)isoxazolidine-2-carbonyl]cyclohexyl]methyl]pyrazolo[4,3-b]pyridine-6-carbonitrile (Compound (186)) is performed as in STEP 3a here-above but starting from (3S)-3-(6-methoxypyrazin-2-yl)isoxazolidine instead of 3-fluoro-5-[(3S)-isoxazolidin-3-yl]benzonitrile hydrochloride.

STEP 3f: Synthesis of trans-1-[[4-[(3S)-3-(2-pyridyl)isoxazolidine-2-carbonyl]cyclohexyl]methyl]pyrazolo[4,3-b]pyridine-6-carbonitrile (Compound (187)) is performed as in STEP 3a here-above but starting from (3S)-3-(2-pyridyl)isoxazolidine instead of 3-fluoro-5-[(3S)-isoxazolidin-3-yl]benzonitrile hydrochloride.

STEP 3g: Synthesis of trans-1-[[4-[(3S)-3-(2-methylthiazol-4-yl)isoxazolidine-2-carbonyl]cyclohexyl]methyl]pyrazolo[4,3-b]pyridine-6-carbonitrile (Compound (199)) is performed as in STEP 3a here-above but starting from (3S)-3-(2-methylthiazol-4-yl)isoxazolidine instead of 3-fluoro-5-[(3S)-isoxazolidin-3-yl]benzonitrile hydrochloride.

### Example 3.21: Synthesis of Compound (14)

STEP 1: Synthesis of methyl trans-4-(pyrrolo[3,2-b]pyridin-1-ylmethyl)cyclohexanecarboxylate—heteroaromatic (compound containing a Nitrogen atom used in step 6a of Scheme 3 in a similar way as performed in example 3.1 step 1, but starting from pyrrolo[3,2-b]pyridine

STEP 2: Synthesis of trans-4-(pyrrolo[3,2-b]pyridin-1-ylmethyl)cyclohexanecarboxylic acid—Compound (Iia) in Scheme 1—in a similar way as performed in Example 1.1, but using methyl trans-4-(pyrrolo[3,2-b]pyridin-1-ylmethyl)cyclohexanecarboxylate instead of trans-4-(benzimidazol-1-ylmethyl)cyclohexanecarboxylate

STEP 3: Synthesis of trans-3-((S)-2-(4-((1H-pyrrolo[3,2-b]pyridin-1-yl)methyl)cyclohexane-1-carbonyl)isoxazolidin-3-yl)-5-fluorobenzonitrile (Compound (14)) is performed as in STEP 5c of Example 1.4, but starting from trans-4-(pyrrolo[3,2-b]pyridin-1-ylmethyl)cyclohexanecarboxylic acid instead of trans-4-[(5-cyano-6-fluoro-benzimidazol-1-yl)methyl]cyclohexanecarboxylic acid.

### Example 3.22: Synthesis of Compound (15)

STEP 1: Synthesis of methyl trans-4-(pyrrolo[3,2-c]pyridin-1-ylmethyl)cyclohexanecarboxylate—heteroaromatic compound containing a Nitrogen atom used in step 6a of Scheme 3 in a similar way as performed in example 3.1 step 1, but starting from pyrrolo[3,2-c]pyridine

STEP 2: Synthesis of trans-4-(pyrrolo[3,2-c]pyridin-1-ylmethyl)cyclohexanecarboxylic acid—Compound (Iia) in Scheme 1—in a similar way as performed in Example 1.1, but using methyl trans-4-(pyrrolo[3,2-c]pyridin-1-ylmethyl)cyclohexanecarboxylate instead of trans-4-(benzimidazol-1-ylmethyl)cyclohexanecarboxylate

STEP 3: Synthesis of trans-3-((S)-2-(4-((1H-pyrrolo[3,2-c]pyridin-1-yl)methyl)cyclohexane-1-carbonyl)isoxazolidin-3-yl)-5-fluorobenzonitrile (Compound (15)) is performed as in STEP 5c of Example 1.4, but starting from trans-4-(pyrrolo[3,2-c]pyridin-1-ylmethyl)cyclohexanecarboxylic acid instead of trans-4-[(5-cyano-6-fluoro-benzimidazol-1-yl)methyl]cyclohexanecarboxylic acid.

### Example 3.23: Synthesis of Compound (16)

STEP 1: Synthesis of methyl trans-4-(pyrrolo[2,3-c]pyridin-1-ylmethyl)cyclohexanecarboxylate—heteroaromatic compound containing a Nitrogen atom used in step 6a of Scheme 3 in a similar way as performed in example 3.1 step 1, but starting from pyrrolo[2,3-c]pyridine

STEP 2: Synthesis of trans-4-(pyrrolo[2,3-c]pyridin-1-ylmethyl)cyclohexanecarboxylic acid—Compound (Iia) in Scheme 1—in a similar way as performed in Example 1.1, but using methyl trans-4-(pyrrolo[2,3-c]pyridin-1-ylmethyl)cyclohexanecarboxylate instead of trans-4-(benzimidazol-1-ylmethyl)cyclohexanecarboxylate

STEP 3: Synthesis of trans-3-((S)-2-(4-((1H-pyrrolo[2,3-c]pyridin-1-yl)methyl)cyclohexane-1-carbonyl)isoxazolidin-3-yl)-5-fluorobenzonitrile (Compound (16)) is performed as in STEP 5c of Example 1.4, but starting from trans-4-(pyrrolo[2,3-c]pyridin-1-ylmethyl)cyclohexanecarboxylic acid instead of trans-4-[(5-cyano-6-fluoro-benzimidazol-1-yl)methyl]cyclohexanecarboxylic acid.

### Example 3.24: Synthesis of Compounds (18), (20)

STEP 1: Synthesis of methyl trans-4-[(2-oxo-1,3-benzoxazol-3-yl)methyl]cyclohexanecarboxylate—heteroaromatic compound containing a Nitrogen atom used in step 6a of Scheme 3 in a similar way as performed in example 3.1 step 1, but starting from 2-oxo-1,3-benzoxazole

STEP 2: Synthesis of trans-4-[(2-oxo-1,3-benzoxazol-3-yl)methyl]cyclohexanecarboxylic acid—Compound (Iia) in Scheme 1—in a similar way as performed in Example 1.1, but using methyl trans-4-[(2-oxo-1,3-benzoxazol-3-yl)methyl]cyclohexanecarboxylate instead of trans-4-(benzimidazol-1-ylmethyl)cyclohexanecarboxylate.

STEP 3a: Synthesis of trans-3-fluoro-5-((S)-2-(4-((2-oxobenzo[d]oxazol-3(2H)-yl)methyl)cyclohexane-1-carbonyl)isoxazolidin-3-yl)benzonitrile (Compound (18)) is performed as in STEP 5c of Example 1.4, but starting from trans-4-[(2-oxo-1,3-benzoxazol-3-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-3-((4-((S)-3-(3,5-difluorophenyl)isoxazolidine-2-carbonyl)cyclohexyl)methyl)benzo[d]oxazol-2(3H)-one (Compound (20)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.25: Synthesis of Compounds (19), (21)

STEP 1: Synthesis of methyl trans-4-[(6-chloropyrrolo[3,2-b]pyridin-1-yl)methyl]cyclohexanecarboxylate—heteroaromatic compound containing a Nitrogen atom used in step 6a of Scheme 3 in a similar way as performed in example 3.1 step 1, but starting from 6-chloropyrrolo[3,2-b]pyridine

STEP 2: Synthesis of trans-4-[(6-chloropyrrolo[3,2-b]pyridin-1-yl)methyl]cyclohexanecarboxylic acid—Compound (Iia) in Scheme 1—in a similar way as performed in Example 1.1, but using methyl trans-4-[(6-chloropyrrolo[3,2-b]pyridin-1-yl)methyl]cyclohexanecarboxylate instead of trans-4-(benzimidazol-1-ylmethyl)cyclohexanecarboxylate.

STEP 3a: Synthesis of trans-3-((S)-2-(4-((6-chloro-1H-pyrrolo[3,2-b]pyridin-1-yl)methyl)cyclohexane-1-carbonyl)isoxazolidin-3-yl)-5-fluorobenzonitrile (Compound (19)) is performed as in STEP 5c of Example 1.4, but starting from trans-4-[(6-chloropyrrolo[3,2-b]pyridin-1-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-(4-((6-chloro-1H-pyrrolo[3,2-b]pyridin-1-yl)methyl)cyclohexyl)((S)-3-(3,5-difluorophenyl)isoxazolidin-2-yl)methanone (Compound (21)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.26: Synthesis of Compounds (22) and (23)

STEP 1: Synthesis of methyl trans-4-[(6-cyano-5-fluoro-indol-1-yl)methyl]cyclohexanecarboxylate—heteroaromatic compound containing a Nitrogen atom used in step 6a of Scheme 3 in a similar way as performed in example 3.1 step 1, but starting from 6-cyano-5-fluoro-indole

STEP 2: Synthesis of trans-4-[(6-cyano-5-fluoro-indol-1-yl)methyl]cyclohexanecarboxylic acid—Compound (Iia) in Scheme 1—in a similar way as performed in Example 1.1, but using methyl trans-4-[(6-cyano-5-fluoro-indol-1-yl)methyl]cyclohexanecarboxylate instead of trans-4-(benzimidazol-1-ylmethyl)cyclohexanecarboxylate.

STEP 3a: Synthesis of trans-1-((4-((S)-3-(3-cyano-5-fluorophenyl)isoxazolidine-2-carbonyl)cyclohexyl)methyl)-5-fluoro-1H-indole-6-carbonitrile (Compound (22)) is performed as in STEP 5c of Example 1.4, but starting from trans-4-[(6-cyano-5-fluoro-indol-1-yl)methyl]cyclohexanecarboxylic acid instead of trans-4-[(5-cyano-6-fluoro-benzimidazol-1-yl)methyl]cyclohexanecarboxylic acid. STEP 3b: Synthesis of trans-1-((4-((S)-3-(3,5-difluorophenyl)isoxazolidine-2-carbonyl)cyclohexyl)methyl)-5-fluoro-1H-indole-6-carbonitrile (Compound (23)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.27: Synthesis of Compounds (24) and (25)

STEP 1: Synthesis of methyl trans-4-[(6-cyanopyrazolo[4,3-b]pyridin-2-yl)methyl]cyclohexanecarboxylate—heteroaromatic compound containing a Nitrogen atom used in step 6a of Scheme 3 in a similar way as performed in example 3.1 step 1, but starting from 6-cyanopyrazolo[4,3-b]pyridine

STEP 2: Synthesis of trans-4-[(6-cyanopyrazolo[4,3-b]pyridin-2-yl)methyl]cyclohexane carboxylic acid—Compound (Iia) in Scheme 1—in a similar way as performed in Example 1.1, but using methyl trans-4-[(6-cyanopyrazolo[4,3-b]pyridin-2-yl)methyl]cyclohexanecarboxylate instead of trans-4-(benzimidazol-1-ylmethyl)cyclohexanecarboxylate.

STEP 3a: Synthesis of trans-2-((4-((S)-3-(3-cyano-5-fluorophenyl)isoxazolidine-2-carbonyl)cyclohexyl)methyl)-2H-pyrazolo[4,3-b]pyridine-6-carbonitrile (Compound (24)) is performed as in STEP 5c of Example 1.4, but starting from trans-4-[(6-cyanopyrazolo[4,3-b]pyridin-2-yl)methyl]cyclohexane carboxylic acid instead of trans-4-[(5-cyano-6-fluoro-benzimidazol-1-yl)methyl]cyclohexanecarboxylic acid.

STEP 3b: Synthesis of trans-2-((4-((S)-3-(3,5-difluorophenyl)isoxazolidine-2-carbonyl)cyclohexyl)methyl)-2H-pyrazolo[4,3-b]pyridine-6-carbonitrile (Compound (25)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 3.28: Synthesis of Compound (120)

STEP 1: Synthesis of methyl trans-4-[(5-methyloxy-pyrrolo[3,2-b]pyridin-1-yl)methyl]cyclohexanecarboxylate—heteroaromatic compound containing a Nitrogen atom used in step 6a of Scheme 3 in a similar way as performed in example 3.1 step 1, but starting from 5-methyloxy-pyrrolo[3,2-b]pyridine

STEP 2: Synthesis of trans-4-[(5-methyloxy-pyrrolo[3,2-b]pyridin-1-yl)methyl]cyclohexanecarboxylic acid

To a mixture of methyl trans-4-[(5-methyloxy-pyrrolo[3,2-b]pyridin-1-yl)methyl]cyclo-hexanecarboxylate (1 g, 3.31 mmol, 1 eq.) in THF (2 ml) and water (4 ml) was added LiOH·H₂O (1 M, 6.61 ml, 2 eq.) and the mixture was stirred at 25° C. for 4 hrs. The reaction mixture was adjusted to pH 3 by addition of 1 M HCl, and then filtered and concentrated under reduced pressure. The crude product was triturated with acetonitrile at 25° C. for 20 min to give the title compound as a white solid (700 mg, 2.43 mmol, 73% yield), which was used without further purification.

STEP 3: Synthesis of trans-3-fluoro-5-[(3S)-2-[4-[(5-methoxypyrrolo[3,2-b]pyridin-1-yl)methyl]cyclohexanecarbonyl]isoxazolidin-3-yl]benzonitrile (Compound (120)) is performed as in STEP 5c of Example 1.4, but starting from trans-4-[(5-methyloxy-pyrrolo[3,2-b]pyridin-1-yl)methyl]cyclohexanecarboxylic acid instead of trans-4-[(5-cyano-6-fluoro-benzimidazol-1-yl)methyl]cyclohexanecarboxylic acid.

### Example 3.29: Synthesis of Compound (128)

STEP 1: Synthesis of methyl trans-4-[(6-methyloxy-pyrrolo[3,2-b]pyridin-1-yl)methyl]cyclohexanecarboxylate—heteroaromatic compound containing a Nitrogen atom used in step 6a of Scheme 3 in a similar way as performed in example 3.1 step 1, but starting from 6-methyloxy-pyrrolo[3,2-b]pyridine

STEP 2: Synthesis of trans-4-[(6-methoxypyrrolo[3,2-b]pyridin-1-yl)methyl]cyclohexanecarboxylic acid—Compound (Iia) in Scheme 1—in a similar way as performed in Example 3.28, but using methyl trans-4-[(6-methyloxy-pyrrolo[3,2-b]pyridin-1-yl)methyl]cyclohexanecarboxylate instead of methyl trans-4-[(5-methyloxy-pyrrolo[3,2-b]pyridin-1-yl)methyl]cyclohexanecarboxylate

STEP 3: Synthesis of trans-3-fluoro-5-[(3S)-2-[4-[(6-methoxypyrrolo[3,2-b]pyridin-1-yl)methyl]cyclohexanecarbonyl]isoxazolidin-3-yl]benzonitrile (Compound (128)) is performed as in STEP 5c of Example 1.4, but starting from trans-4-[(6-methoxypyrrolo[3,2-b]pyridin-1-yl)methyl]cyclohexanecarboxylic acid instead of trans-4-[(5-cyano-6-fluoro-benzimidazol-1-yl)methyl]cyclohexanecarboxylic acid.

### Example 3.30: Synthesis of Compound (241)

STEP 1: Synthesis of methyl trans-4-[(6-cyanoindol-1-yl)methyl]cyclohexanecarboxylate is performed as in STEP 1 of Example 3.1, but starting from 6-cyano-indol and using methyl trans-4-[(4-nitrophenyl)sulfonyloxymethyl]cyclohexanecarboxylate for alkylation (step 6a of Scheme 3)

STEP 2: is performed as in STEP 2 of Example 3.1.

STEP 3: Synthesis of trans-1-[[4-[(3S)-3-(6-methoxypyrazin-2-yl)isoxazolidine-2-carbonyl]cyclohexyl]methyl]indole-6-carbonitrile (compound (241)) is performed as in STEP 5c of Example 1.4, but starting from trans-4-[(6-cyanoindol-1-yl)methyl]cyclohexanecarboxylic acid and (3S)-3-(6-methoxypyrazin-2-yl)isoxazolidine.

### Example 4: Synthesis of Compounds (129), (146), (149), (152), (153)

### Example 4.1: Synthesis of Compound (129)

### STEP 1: Synthesis of Methyl trans-4-[(6-hydroxypyrrolo[3,2-b]pyridin-1-yl)methyl]cyclohexanecarboxylate—Compound (IIIb) in Scheme 3

### Step a: Synthesis of Methyl trans-4-(p-tolylsulfonyloxymethyl)cyclohexanecarboxylate

To a solution of methyl trans-4-(hydroxymethyl)cyclohexanecarboxylate (11.28 g, 65.50 mmol, 1 eq.) in DCM (120 ml) was added DMAP (4.00 g, 32.75 mmol, 0.5 eq.), TEA (7.29 g, 72.05 mmol, 10.03 ml, 1.1 eq.) and TosCl (13.49 g, 70.74 mmol, 1.08 eq.) at 25° C. The mixture was stirred at 25° C. for 12 hrs. The reaction mixture was concentrated and the residue was purified by column chromatography (SiO₂, petroleum ether/EA=1/1 to 2/1) to give the title compound (14 g, 41.08 mmol, 63% yield) as yellow solid.

LC/MS: m/z 327.2 [M+H]⁺ (Method B)

### Step b: Synthesis of Methyl trans-4-[(6-bromopyrrolo[3,2-b]pyridin-1-yl)methyl]cyclohexanecarboxylate

A mixture of 6-bromo-1H-pyrrolo[3,2-b]pyridine (2.85 g, 14.48 mmol, 1 eq.), methyl trans-4-(p-tolylsulfonyloxymethyl)cyclohexanecarboxylate (5.67 g, 17.37 mmol, 1.2 eq.) and Cs₂CO₃ (9.43 g, 28.95 mmol, 2 eq.) in DMF (20 ml) was degassed, purged with N₂ and stirred at 80° C. for 3 hrs. The residue was diluted with water (100 ml) and extracted with EA (100 ml×2). The combined organic layers were dried over Na₂SO₄, filtered and concentrated under reduced pressure. The residue was purified by flash silica gel chromatography (ISCO®; 20 g SepaFlash® Silica Flash Column, mobile phase: petroleum ether and EA, gradient: 0-50% EA, flow: 80 ml/min) to give the title compound (4.43 g, 11.60 mmol, 80% yield) as a yellow oil.

LC/MS: m/z 351.0/353.0 [M+H]⁺ (Method B)

¹H NMR (400 MHz, CDCl₃): δ ppm 8.49 (d, J=1.8 Hz, 1H), 7.76 (d, J=1.1 Hz, 1H), 7.27-7.26 (m, 1H), 6.68 (d, J=2.8 Hz, 1H), 3.93 (d, J=7.2 Hz, 2H), 3.66 (s, 3H), 2.32-2.19 (m, 1H), 2.04-1.96 (m, 2H), 1.90-1.76 (m, 1H), 1.76-1.66 (m, 2H), 1.40 (dq, J=3.3, 12.9 Hz, 2H), 1.11-0.98 (m, 2H).

### Step c: Synthesis of Methyl trans-4-[[6-(4,4,5,5-tetramethyl-1,3,2-dioxaborolan-2-yl)pyrrolo[3,2-b]pyridin-1-yl]methyl]cyclohexanecarboxylate

A mixture of methyl trans-4-[(6-bromopyrrolo[3,2-b]pyridin-1-yl)methyl]cyclohexane-carboxylate (4.43 g, 12.61 mmol, 1 eq.), BPD (3.52 g, 13.87 mmol, 1.1 eq.), KOAc (3.71 g, 37.84 mmol, 3 eq.) and Pd(dppf)Cl₂·CH₂Cl₂ (1.03 g, 1.26 mmol, 0.1 eq.) in dioxane (40 ml) was degassed and purged with N₂ and stirred at 90° C. for 2 hrs under N₂ atmosphere. The residue was diluted with water (60 ml) and extracted with EA (60 ml×2). The combined organic layers were dried over Na₂SO₄, filtered and concentrated under reduced pressure and the residue was purified by flash silica gel chromatography (ISCO®; 8 g SepaFlash® Silica Flash Column, mobile phase: petroleum ether and EA, gradient 0˜50% EA, flow: 40 ml/min) to give the title compound (3.82 g, 9.56 mmol, 76% yield) as a yellow oil.

LC/MS: m/z 317.2 [M-81]⁺ (Method B)

¹H NMR (400 MHz, CDCl₃): δ ppm 8.78-8.41 (m, 1H), 8.33-8.00 (m, 1H), 7.20 (br s, 1H), 4.13 (q, J=7.1 Hz, 2H), 3.65-3.57 (m, 3H), 2.24 (dt, J=4.4, 8.8 Hz, 1H), 1.92-1.81 (m, 2H), 1.80-1.70 (m, 2H), 1.40 (s, 2H), 1.27 (t, J=7.1 Hz, 3H).

### Step d: Synthesis of Methyl trans-4-[(6-hydroxypyrrolo[3,2-b]pyridin-1-yl)methyl]cyclohexanecarboxylate

To a solution of methyl trans-4-[[6-(4,4,5,5-tetramethyl-1,3,2-dioxaborolan-2-yl)pyrrolo[3,2-b]pyridin-1-yl]methyl]cyclohexanecarboxylate (3.62 g, 9.09 mmol, 1 eq.) in THF (32 ml) sodium perborate tetrahydrate (5.59 g, 36.35 mmol, 6.99 ml, 4 eq.) and water (8 ml) was added dropwise. After addition, the resulting mixture was stirred at 55° C. for 2 hrs. The residue was diluted with water (60 ml) and extracted with EA (60 ml×2). The combined organic layers were dried over Na₂SO₄, filtered and concentrated under reduced pressure. The crude product was triturated with petroleum ether/EA (3/1) at 25° C. for 60 min to give the title compound (2 g, 6.93 mmol, 76% yield) as a gray solid.

LC/MS: m/z 289.2 [M+H]⁺ (Method B)

¹H NMR (400 MHz, CDCl₃): δ ppm 10.96-9.57 (m, 1H), 8.43 (d, J=2.3 Hz, 1H), 7.24 (d, J=1.6 Hz, 1H), 7.16 (d, J=3.3 Hz, 1H), 6.64 (d, J=2.8 Hz, 1H), 3.89 (d, J=7.1 Hz, 2H), 3.66 (s, 3H), 2.24 (tt, J=3.5, 12.2 Hz, 1H), 2.05-1.93 (m, 2H), 1.90-1.78 (m, 1H), 1.76-1.67 (m, 2H), 1.43-1.31 (m, 2H), 1.14-0.96 (m, 2H).

### STEP 2: Synthesis of trans-4-[(6-benzyloxypyrrolo[3,2-b]pyridin-1-yl)methyl]cyclohexanecarboxylic Acid

### Step a: Synthesis of 1H-pyrrolo[3,2-b]pyridine-1-ylethanone

A mixture of 1H-pyrrolo[3,2-b]pyridine (22.7 g, 192.15 mmol, 1 eq), Ac₂O (58.85 g, 576.45 mmol, 53.99 mL, 3 eq), DMAP (1.17 g, 9.61 mmol, 0.05 eq) in THF (230 mL) was stirred at 25° C. for 2 hr. TLC indicated compound 1 was consumed and one new spot formed. The mixture was concentrated, diluted with water (500 mL), extracted with EtOAc (500 mL*3), wash with sat. NaHCO₃ solution (300 mL), dried with Na₂SO₄, filtered and concentrated to give 1H-pyrrolo[3,2-b]pyridine-1-ylethanone (30.7 g, 99.75% yield) as a yellow solid.

¹H NMR (CDCl₃400 MHz): δ ppm 8.68 (1H, brd, J=8.3 Hz), 8.57 (1H, dd, J=1.5 and 4.8 Hz), 7.68 (1H, d, J=3.8 Hz), 7.27 (1H, s), 6.87 (1H, d, J=3.9 Hz), 2.67 (3H, s)

### Step b: Synthesis of 1(4-hydroxopyrrolo[3,2-b]pyridine-1-yl)ethanone

To a solution of 1H-pyrrolo[3,2-b]pyridine-1-ylethanone (28.7 g, 179.18 mmol, 1 eq) in DCM (300 mL) was added m-CPBA (43.65 g, 215.02 mmol, 85% purity, 1.2 eq). The reaction mixture was stirred at 25° C. for 12 hr. TLC indicated starting material was consumed and one new major spot formed. The reaction was concentrated. The residue was purified by column chromatography (SiO₂, DCM:MeOH=10:1) to give 1(4-hydroxopyrrolo[3,2-b]pyridine-1-yl)ethanone (36 g, crude) as a yellow solid.

¹H NMR (CDCl₃, 400 MHz): δ ppm 8.25-8.45 (2H, m), 7.62 (1H, br s), 7.27 (1H, s), 7.18 (1H, br s), 2.64-2.73 (3H, m).

### Step c: Synthesis of 1H-pyrrolo[3,2-b]pyridin-6-ol

A mixture of 1(4-hydroxopyrrolo[3,2-b]pyridine-1-yl)ethanone (31 g, 123.18 mmol, 70% purity, 1 eq) in Ac₂O (240 mL) was stirred at 130° C. for 12 hr. LCMS showed sm was consumed and desired MS was detected. The reaction mixture was concentrated. The residue was in MeOH (300 mL) was added K₂CO₃ (28.50 g, 206.22 mmol, 1.5 eq). The mixture was stirred at 25° C. for 1 hr. The reaction mixture was filtered and concentrated. The residue was purified by silica gel column chromatography (SiO2, DCM:MeOH=10:1) to give 1H-pyrrolo[3,2-b]pyridin-6-ol (16 g, crude) as a yellow oil.

¹H NMR (MeOD, 400 MHz): δ ppm 7.97 (1H, d, J=2.4 Hz), 7.38 (1H, d, J=3.3 Hz), 7.25 (1H, d, J=3.1 Hz), 6.48 (1H, d, J=2.8 Hz).

### Step d: Synthesis of c-benzyloxy-1H-pyrrolo[3,2-b]pyridine

To a solution of 1H-pyrrolo[3,2-b]pyridin-6-ol (9 g, 67.10 mmol, 1 eq) and BnBr (11.48 g, 67.10 mmol, 7.97 mL, 1 eq) in DMF (100 mL) was added CS₂CO₃ (32.79 g, 100.64 mmol, 1.5 eq) under N₂. The reaction mixture was stirred at 25° C. for 2 hours. LC-MS (EW23468-84-P1A1) showed Reactant 1 consumed completely. Several new peaks were shown on LC-MS and 29% of desired mass was detected (Rt=0.733 min). The reaction mixture was poured into H₂O (1000 mL) and extracted with EA (100 mL*3). The combined organic layer was washed with brine (1000 mL), dried over Na₂SO₄, filtered and concentrated. The crude product was purified by reversed-phase HPLC (0.1% FA condition). 6-benzyloxy-1H-pyrrolo[3,2-b]pyridine (2 g, 8.92 mmol, 13.29% yield) was obtained as a white solid.

¹H NMR (400 MHz, CHLOROFORM-d) δ=8.47 (br s, 1H), 8.39 (d, J=2.6 Hz, 1H), 7.50-7.46 (m, 2H), 7.45-7.39 (m, 2H), 7.38-7.33 (m, 2H), 7.25 (dd, J=0.7, 2.4 Hz, 1H), 6.71 (ddd, J=0.8, 2.1, 3.2 Hz, 1H), 5.16 (s, 2H).

Compound (Iia) in Scheme 1—in a similar way as performed as in Example 3.28. starting from 6-(Benzyloxy)-1H-pyrrolo[2,3-b]pyridine 6-benzyloxypyrrolo[3,2-b]pyridine.

### STEP 3: Synthesis of trans-4-[(6-hydroxypyrrolo[3,2-b]pyridin-1-yl)methyl]cyclohexanecarboxylic Acid

To a solution of trans-4-[(6-benzyloxypyrrolo[3,2-b]pyridin-1-yl)methyl]cyclohexane-carboxylic acid (1 g, 2.74 mmol, 1 eq.) in MeOH (20 ml) was added Pd/C (100 mg, 2.74 mmol, 10% purity, 1 eq.) under N₂. The suspension was degassed under vacuum and purged with H₂ several times. The mixture was stirred under H₂ (15 psi) at 25° C. for 3 hrs. The reaction mixture was filtered and concentrated. The crude product was obtained as a white solid (750 mg, 2.73 mmol, 100% yield) and used without further purification.

STEP 4: Synthesis of trans-3-fluoro-5-[(3S)-2-[4-[(6-hydroxypyrrolo[3,2-b]pyridin-1-yl)methyl]cyclohexanecarbonyl]isoxazolidin-3-yl]benzonitrile (Compound (129)) is performed as in STEP 5c of Example 1.4, but starting from trans-4-[(6-hydroxypyrrolo[3,2-b]pyridin-1-yl)methyl]cyclohexanecarboxylic acid instead of trans-4-[(5-cyano-6-fluoro-benzimidazol-1-yl)methyl]cyclohexanecarboxylic acid.

### Example 4.2: Synthesis of Compound (146)

Methyl trans-4-[(6-hydroxypyrrolo[3,2-b]pyridin-1-yl)methyl]cyclohexane-carboxylate can be synthesized as detailed in STEP 1 of Example 4.1 here-above.

### STEP 1: Synthesis of Methyl trans-4-[[6-(oxetan-3-yloxy)pyrrolo[3,2-b]pyridin-1-yl]methyl]cyclo-hexanecarboxylate—Compound (IIIb) in Scheme 3

To a solution of methyl trans-4-[(6-hydroxypyrrolo[3,2-b]pyridin-1-yl)methyl]cyclohexane-carboxylate (200 mg, 693.62 μmol, 1 eq.) and 3-iodooxetane (191.41 mg, 1.04 mmol, 1.5 eq.) in DMSO (2 ml) t-BuOK (116.75 mg, 1.04 mmol, 1.5 eq.) was added. The mixture was stirred at 20° C. for 2 hrs. The reaction mixture was filtered and concentrated under reduced pressure to give the title compound (500 mg, crude) as a black oil.

STEP 2: Synthesis of trans-4-[[6-(oxetan-3-yloxy)pyrrolo[3,2-b]pyridin-1-yl]methyl]cyclohexanecarboxylic acid—Compound (Iia) in Scheme 1—is performed in a similar way as performed in Example 1.1, but using methyl trans-4-[[6-(oxetan-3-yloxy)pyrrolo[3,2-b]pyridin-1-yl]methyl]cyclo-hexanecarboxylate instead of trans-4-(benzimidazol-1-ylmethyl)cyclohexanecarboxylate

STEP 3a: Synthesis of trans-3-fluoro-5-[(3S)-2-[4-[[6-(oxetan-3-yloxy)pyrrolo[3,2-b]pyridin-1-yl]methyl]cyclohexanecarbonyl]isoxazolidin-3-yl]benzonitrile (Compound (146)) is performed as in STEP 5c of Example 1.4, but starting from trans-4-[[6-(oxetan-3-yloxy)pyrrolo[3,2-b]pyridin-1-yl]methyl]cyclohexanecarboxylic acid instead of trans-4-[(5-cyano-6-fluoro-benzimidazol-1-yl)methyl]cyclohexanecarboxylic add.

### Example 4.3: Synthesis of Compound (149)

STEP 1: Synthesis of methyl trans-4-[[6-(2-methoxyethoxy)pyrrolo[3,2-b]pyridin-1-yl]methyl]cyclohexanecarboxylate—Compound (IIIb) in Scheme 3—is performed in a similar way as in Example 4.1 here-above

STEP 2: Synthesis of trans-4-[[6-(2-methoxyethoxy)pyrrolo[3,2-b]pyridin-1-yl]methyl]cyclohexanecarboxylic acid—Compound (Iia) in Scheme 1—in a similar way as performed in Example 3.28, but using methyl trans-4-[[6-(2-methoxyethoxy)pyrrolo[3,2-b]pyridin-1-yl]methyl]cyclohexanecarboxylate instead of methyl trans-4-[(5-methyloxy-pyrrolo[3,2-b]pyridin-1-yl)methyl]cyclohexanecarboxylate

STEP 3a: Synthesis of trans-3-fluoro-5-[(3S)-2-[4-[[6-(2-methoxyethoxy)pyrrolo[3,2-b]pyridin-1-yl]methyl]cyclohexanecarbonyl]isoxazolidin-3-yl]benzonitrile (Compound (149)) is performed as in STEP 5c of Example 1.4, but starting from trans-4-[[6-(2-methoxyethoxy)pyrrolo[3,2-b]pyridin-1-yl]methyl]cyclohexanecarboxylic acid instead of trans-4-[(5-cyano-6-fluoro-benzimidazol-1-yl)methyl]cyclohexanecarboxylic acid.

### Example 4.4: Synthesis of Compound (152)

Methyl trans-4-[(6-hydroxypyrrolo[3,2-b]pyridin-1-yl)methyl]cyclohexane-carboxylate can be synthesized as detailed in STEP 1 of Example 4.1 here-above.

STEP 1: Synthesis of methyl trans-4-[[6-(cyclopropylmethoxy)pyrrolo[3,2-b]pyridin-1-yl]methyl]cyclohexanecarboxylate—Compound (IIIb) in Scheme 3 is performed in a similar way as detailed in Example 4.1 using iodomethylcyclopropyl

STEP 2: Synthesis of trans-4-[[6-(cyclopropylmethoxy)pyrrolo[3,2-b]pyridin-1-yl]methyl]cyclohexanecarboxylic acid—Compound (Iia) in Scheme 1—in a similar way as performed in Example 3.28, but using methyl trans-4-[[6-(cyclopropylmethoxy)pyrrolo[3,2-b]pyridin-1-yl]methyl]cyclohexanecarboxylate instead of methyl trans-4-[(5-methyloxy-pyrrolo[3,2-b]pyridin-1-yl)methyl]cyclohexanecarboxylate

STEP 3: Synthesis of trans-3-[(3S)-2-[4-[[6-(cyclopropylmethoxy)pyrrolo[3,2-b]pyridin-1-yl]methyl]cyclohexanecarbonyl]isoxazolidin-3-yl]-5-fluoro-benzonitrile (Compound (152)) is performed as in STEP 5c of Example 1.4, but starting from trans-4-[[6-(cyclopropylmethoxy)pyrrolo[3,2-b]pyridin-1-yl]methyl]cyclohexanecarboxylic acid instead of trans-4-[(5-cyano-6-fluoro-benzimidazol-1-yl)methyl]cyclohexanecarboxylic acid.

### Example 4.5: Synthesis of Compound (153)

Methyl trans-4-[(6-hydroxypyrrolo[3,2-b]pyridin-1-yl)methyl]cyclohexane-carboxylate can be synthesized as detailed in STEP 1 of Example 4.1 here-above.

STEP 1: Synthesis of methyl trans-4-[[6-(2-hydroxyethoxy)pyrrolo[3,2-b]pyridin-1-yl]methyl]cyclo-hexanecarboxylate—Compound (IIIb) in Scheme 3

Step a: synthesis of methyl trans-4-[[6-[2-[tert-butyl(dimethyl)silyl]oxyethoxy]pyrrolo[3,2-b]pyridin-1-yl]methyl]cyclohexanecarboxylate, starting from Methyl trans-4-[(6-hydroxypyrrolo[3,2-b]pyridin-1-yl)methyl]cyclohexane-carboxylate as detailed in example 4.2 here-above.

Step b: A mixture of methyl trans-4-[[6-[2-[tert-butyl(dimethyl)silyl]oxyethoxy]pyrrolo[3,2-b]pyridin-1-yl]methyl]cyclohexanecarboxylate (300 mg, 671.66 μmol, 1 eq.) and TBAF (324.83 mg, 2.01 mmol, 328.45 μl, 3 eq.) in THF (3 ml) was stirred at 25° C. for 1 hr. The reaction mixture was diluted with water (10 ml) and extracted with EA (10 ml), the combined organic layers were washed with brine (10 ml), dried over Na₂SO₄, filtered and concentrated under reduced pressure to give the title compound (234 mg, crude) as a yellow solid.

LC/MS: m/z 333.2 [M+H]⁺ (Method B)

STEP 2: Synthesis of trans-4-[[6-(2-hydroxyethoxy)pyrrolo[3,2-b]pyridin-1-yl]methyl]cyclohexanecarboxylic acid—Compound (Iia) in Scheme 1—in a similar way as performed in Example 3.28, but using methyl trans-4-[[6-(2-hydroxyethoxy)pyrrolo[3,2-b]pyridin-1-yl]methyl]cyclo-hexanecarboxylate instead of methyl trans-4-[(5-methyloxy-pyrrolo[3,2-b]pyridin-1-yl)methyl]cyclohexanecarboxylate

STEP 3: Synthesis of trans-3-fluoro-5-[(3S)-2-[4-[[6-(2-hydroxyethoxy)pyrrolo[3,2-b]pyridin-1-yl]methyl]cyclohexanecarbonyl]isoxazolidin-3-yl]benzonitrile (Compound (153)) is performed as in STEP 5c of Example 1.4, but starting from trans-4-[[6-(2-hydroxyethoxy)pyrrolo[3,2-b]pyridin-1-yl]methyl]cyclohexanecarboxylic acid instead of trans-4-[(5-cyano-6-fluoro-benzimidazol-1-yl)methyl]cyclohexanecarboxylic acid.

### Example 5: Synthesis of Compound (302) and (398)

### STEP 1: Synthesis of Methyl trans-4-[(6-methylpyridazin-4-yl)methyl]cyclohexanecarboxylate—Compound (IIIb) in Scheme 3

The reaction mixture of methyl trans-4-[(6-chloropyridazin-4-yl)methyl]cyclohexane-carboxylate (300.0 mg, 1.12 mmol, 1.0 eq.), 1,1′-bis(diphenylphosphino)ferrocene-palladium-(II)dichloride dichloromethane complex (91.2 mg, 0.11 mmol, 0.1 eq.), Cs₂CO₃ (1.64 g, 5.02 mmol, 4.5 eq.), water (2 ml) and trimethylboroxin (50% in THF, 840.8 mg, 0.941 ml, 3.35 mmol, 3 eq.) was degassed with argon for 5 min and stirred at 80° C. for 9 hrs. Trimethylboroxin (50% in THF, 0.50 ml), 1,1′-bis(diphenylphosphino)ferrocene-palladium(II)dichloride dichloromethane complex (50 mg) and a spatula tip of Cs₂CO₃ were added and the reaction mixture was stirred again for 6.5 hrs. Trimethylboroxin (50% in THF, 0.50 ml), 1,1′-bis(diphenylphosphino)ferrocene-palladium(II)dichloride dichloromethane complex (50 mg) and a spatula tip of Cs₂CO₃ were added again and the reaction mixture was stirred again for 3 hrs. The solvent was evaporated under reduced pressure and the residue was quenched with EA and water (30 ml each). The water phase was extracted with EA (20 ml). The combined organic layers were dried over MgSO₄, evaporated and purified by SiO₂ chromatography (24 g SO₂, eluent: heptane/EA, gradient: 100% heptane for 5 min, 0-100% EA in 30 min, 100% EA for 10 min, flow: 40 ml/min) to give the title compound (125 mg, 0.50 mmol, 45% yield).

¹H NMR (400 MHz, DMSO-d₆): δ ppm 8.90 (d, J=1.96 Hz, 1H), 7.36 (d, J=1.83 Hz, 1H), 3.57 (s, 3H), 2.57 (s, 3H), 2.25 (m, 1H), 1.89 (m, 2H), 1.59 (m, 3H), 1.27 (m, 3H), 1.00 (m, 2H), 0.85 (br t, J=6.91, 6.91 Hz, 1H).

STEP 2: Synthesis of trans-4-[(6-methylpyridazin-4-yl)methyl]cyclohexanecarboxylic acid—Compound (Iia) in Scheme 1—in a similar way as performed in Example 1.1, but using methyl trans-4-[(6-methylpyridazin-4-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(6-methylpyridazin-4-yl)methyl]cyclohexanecarbonyl]isoxazolidin-3-yl]benzonitrile (Compound (302)) is performed as in STEP 5c of Example 1.4, but starting from trans-4-[(6-methylpyridazin-4-yl)methyl]cyclohexanecarboxylic acid instead of trans-4-[(5-cyano-6-fluoro-benzimidazol-1-yl)methyl]cyclohexanecarboxylic acid.

Step 3b: Synthesis of trans-[(3S)-3-(5-fluoro-6-methylpyridin-3-yl)-1,2-oxazolidin-2-yl]-[4-[(6-methylpyridazin-4-yl)methyl]cyclohexyl]methanone (Compound (398)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile

Chiral SFC: RT 1.26 min (100%, Method X)

### Example 6: Synthesis of Compound (184)

### STEP 1: Synthesis of Methyl trans-4-[[6-(1-methylcyclopropoxy)pyrrolo[3,2-b]pyridin-1-yl]methyl]cyclohexanecarboxylate—Compound (IIIb) in Scheme 3

### Step a: Synthesis of 1-(benzenesulfonyl)-6-bromo-pyrrolo[3,2-b]pyridine

To a solution of NaH (223.29 mg, 5.58 mmol, 60% purity, 1.1 eq.) in THF (6 ml) 6-bromo-1H-pyrrolo[3,2-b]pyridine (1 g, 5.08 mmol, 1 eq.) was added dropwise at 0° C. After stirring for 15 min at 0° C. a solution of benzenesulfonyl chloride (986.05 mg, 5.58 mmol, 714.53 μl, 1.1 eq.) in THF (4 ml) was added dropwise at 0° C. The resulting mixture was stirred at 25° C. for 18 hrs. The residue was diluted with water (30 ml) and extracted with EA (30 ml×2). The combined organic layers were dried over Na₂SO₄, filtered, concentrated under reduced pressure and purified by flash silica gel chromatography (ISCO®; 4 g SepaFlash® Silica Flash Column, eluent petroleum ether and EA, gradient 0˜40% EA, flow 30 ml/min). The title compound (1.65 g, 4.40 mmol, 87% yield) was obtained as a white solid.

LC/MS: m/z 337.0 [M+H]⁺ (Method B)

### Step b: Synthesis of 1-(benzenesulfonyl)-6-(4,4,5,5-tetramethyl-1,3,2-dioxaborolan-2-yl)pyrrolo[3,2-b]pyridine

1-(benzenesulfonyl)-6-bromo-pyrrolo[3,2-b]pyridine (5 g, 14.83 mmol, 1 eq.), BPD (4.90 g, 19.28 mmol, 1.3 eq.), Pd(dppf)Cl₂ (1.08 g, 1.48 mmol, 0.1 eq.) and KOAc (2.91 g, 29.66 mmol, 2 eq.) in dioxane (50 ml) was degassed and heated to 90° C. under N₂ for 12 hrs. The reaction mixture was poured into saturated NH₄Cl (300 ml), extracted with EA (100 ml×3), combined organic layers were washed with brine (200 ml), dried over Na₂SO₄, filtered, concentrated and purified by column chromatography (SiO₂, petroleum ether/EA=5/1 to 1/1). The title compound (4.2 g, 10.93 mmol, 74% yield) was obtained as a white solid.

LC/MS: m/z 385.3 [M+H]⁺ (Method B)

### Step c: Synthesis of 1-(benzenesulfonyl)pyrrolo[3,2-b]pyridin-6-ol

1-(benzenesulfonyl)-6-(4,4,5,5-tetramethyl-1,3,2-dioxaborolan-2-yl)pyrrolo[3,2-b]pyridine (4 g, 10.41 mmol, 1 eq.) and sodium perborate tetrahydrate (6.41 g, 41.64 mmol, 8.01 ml, 4 eq.) in THF (40 ml) and water (10 ml) was degassed and then heated to 55° C. under N₂ for 2 hrs. The reaction mixture was poured into saturated NaHCO₃ aqueous solution (100 ml), extracted with EA (100 ml×3), the combined organic layers were washed with brine (100 ml), dried over Na₂SO₄, filtered, concentrated and used for next step directly. The title compound (2.8 g, 10.21 mmol, 98% yield) was obtained as a white solid.

LC/MS: m/z 275.1 [M+H]⁺ (Method B)

### Step d: Synthesis of methyl 2-[1-(benzenesulfonyl)pyrrolo[3,2-b]pyridin-6-yl]oxy-4-bromo-butanoate

To a mixture of K₂CO (2.32 g, 16.77 mmol, 2 eq.) and methyl 2,4-dibromobutanoate (3.27 g, 12.58 mmol, 1.5 eq.) in DMF (15 ml) 1-(benzenesulfonyl)pyrrolo[3,2-b]pyridin-6-ol (2.3 g, 8.39 mmol, 1 eq.) was added in one portion under N₂ and stirred at 25° C. for 2 hrs. The reaction mixture was poured into saturated NH₄Cl aqueous solution (100 ml), extracted with EA (50 ml×3), the combined organic layers were washed with brine (100 ml), dried over Na₂SO₄, filtered, concentrated and purified by column chromatography (SiO₂, petroleum ether/EA=10/1 to 1/1) to give the title compound (2.6 g, 5.74 mmol, 68% yield) as a yellow oil.

LC/MS: m/z 455.0 [M+H]⁺ (Method B)

### Step e: Synthesis of methyl 1-[1-(benzenesulfonyl)pyrrolo[3,2-b]pyridin-6-yl]oxycyclopropanecarboxylate

To a mixture of t-BuOK (707.97 mg, 6.31 mmol, 1.1 eq.) in THF (60 ml) methyl 2-[1-(benzenesulfonyl)pyrrolo[3,2-b]pyridin-6-yl]oxy-4-bromo-butanoate (2.6 g, 5.74 mmol, 1 eq.) was added in one portion under N₂. The mixture was stirred at 25° C. for 2 hrs, poured into saturated NH₄Cl (100 ml) and extracted with EA (50 ml×3). The combined organic layers were washed with brine (100 ml), dried over Na₂SO₄, filtered, concentrated and purified by column chromatography (SiO₂, petroleum ether/EA=10/1 to 0/1) to give the title compound (1.1 g, 2.95 mmol, 52% yield) as a yellow oil.

¹H NMR (400 MHz, CDCl₃): δ ppm 8.31 (d, J=2.4 Hz, 1H), 7.90-7.80 (m, 3H), 7.71 (d, J=3.7 Hz, 1H), 7.65-7.57 (m, 1H), 7.55-7.46 (m, 2H), 6.83 (d, J=3.8 Hz, 1H), 3.76 (s, 3H), 1.77-1.71 (m, 2H), 1.42-1.36 (m, 2H).

### Step f: Synthesis of [1-[1-(benzenesulfonyl)pyrrolo[3,2-b]pyridin-6-yl]oxycyclopropyl]methanol

To a solution of methyl 1-[1-(benzenesulfonyl)pyrrolo[3,2-b]pyridin-6-yl]oxycyclopropane-carboxylate (1.08 g, 2.90 mmol, 1 eq.) in THF (22 ml) LiAlH₄ (110.07 mg, 2.90 mmol, 1 eq.) was added slowly at 0° C. and stirred at 0° C. for 1 hr. The reaction mixture was quenched by addition of water at 0° C. until there are no bubbles (˜2 ml), dried over Na₂SO₄, filtered and concentrated under reduced pressure to give the title compound (889 mg, crude) as a colorless oil.

LC/MS: m/z 345.0 [M+H]⁺ (Method B)

¹H NMR (400 MHz, CDCl₃): δ ppm 8.32 (d, J=2.5 Hz, 1H), 8.02 (dd, J=0.6, 2.5 Hz, 1H), 7.90-7.86 (m, 2H), 7.69 (d, J=3.8 Hz, 1H), 7.62-7.57 (m, 1H), 7.52-7.46 (m, 2H), 6.80 (dd, J=0.7, 3.8 Hz, 1H), 3.92 (s, 2H), 1.13-1.09 (m, 2H), 1.07-1.02 (m, 2H).

### Step g: Synthesis of 1-(benzenesulfonyl)-6-[1-(iodomethyl)cyclopropoxy]pyrrolo[3,2-b]pyridine

To a mixture of I₂ (655.18 mg, 2.58 mmol, 519.99 μl, 1 eq.) and imidazole (175.74 mg, 2.58 mmol, 1 eq.) in DCM (8 ml) PPh₃ (677.07 mg, 2.58 mmol, 1 eq.) was added at 0° C. for 5 min, then a solution of [1-[1-(benzenesulfonyl)pyrrolo[3,2-b]pyridin-6-yl]oxycyclopropyl]methanol (889 mg, 2.58 mmol, 1 eq.) in DCM (4 ml) was added dropwise to the reaction mixture at 0° C. and stirred at 20° C. for 2 hrs. The reaction mixture was quenched by addition of Na₂SO₃ (5 ml), diluted with water (10 ml), extracted with DCM (10 ml×2), the combined organic layers were dried over Na₂SO₄, filtered, concentrated under reduced pressure and purified by flash silica gel chromatography (eluent: petroleum ether and EA, gradient: 10-40% EA) to give the title compound (829 mg, 1.82 mmol, 71% yield) as a yellow oil.

LC/MS: m/z 454.9 [M+H]⁺ (Method B)

¹H NMR (400 MHz, CDCl₃): δ ppm 8.40 (d, J=2.3 Hz, 1H), 8.00 (d, J=2.3 Hz, 1H), 7.88 (d, J=7.5 Hz, 2H), 7.73 (d, J=3.8 Hz, 1H), 7.64-7.57 (m, 1H), 7.54-7.47 (m, 2H), 6.85 (d, J=3.7 Hz, 1H), 3.53 (s, 2H), 1.52-1.46 (m, 2H), 1.14-1.09 (m, 2H).

### Step h: Synthesis of 1-(benzenesulfonyl)-6-(1-methylcyclopropoxy)pyrrolo[3,2-b]pyridine

To a mixture of 1-(benzenesulfonyl)-6-[1-(iodomethyl)cyclopropoxy]pyrrolo[3,2-b]pyridine (829 mg, 1.82 mmol, 1 eq.) in DMSO (9 ml) NaBH₄ (69.03 mg, 1.82 mmol, 1 eq.) was added and stirred at 90° C. for 1 hr. The reaction mixture was cooled, quenched by addition of NH₄Cl (10 ml), diluted with water (60 ml) and extracted with EA (50 ml×3). The combined organic layers were washed with brine (80 ml×2), dried over Na₂SO₄, filtered and concentrated to give the title compound (650 mg, crude) as a yellow oil.

### Step i: Synthesis of 6-(1-methylcyclopropoxy)-1H-pyrrolo[3,2-b]pyridine

To a solution of 1-(benzenesulfonyl)-6-(1-methylcyclopropoxy)pyrrolo[3,2-b]pyridine (600.00 mg, 1.83 mmol, 1 eq.) in MeOH (6 ml) and THF (3 ml) NaOH (2.5 M, 7.31 ml, 10 eq.) was added and stirred at 20° C. for 1 hr. The reaction mixture was diluted with water (40 ml), extracted with EA (20 ml×3), the combined organic layers were washed with brine (30 ml), dried over Na₂SO₄, filtered, concentrated and purified by flash silica gel chromatography (eluent petroleum ether and EA, gradient 10-35% EA) to give the title compound (200 mg, 1.06 mmol, 58% yield) as a yellow oil.

LC/MS: m/z 189.1 [M+H]⁺ (Method B)

¹H NMR (400 MHz, CDCl₃): δ ppm 8.29 (d, J=2.3 Hz, 2H), 7.39 (dd, J=0.9, 2.4 Hz, 1H), 7.35-7.32 (m, 1H), 6.69 (dt, J=1.0, 2.1 Hz, 1H), 1.59 (s, 3H), 1.08-1.05 (m, 2H), 0.77-0.73 (m, 2H).

### Step j: Synthesis of Methyl trans-4-[[6-(1-methylcyclopropoxy)pyrrolo[3,2-b]pyridin-1-yl]methyl]cyclohexanecarboxylate

A mixture of 6-(1-methylcyclopropoxy)-1H-pyrrolo[3,2-b]pyridine (200 mg, 1.06 mmol, 1 eq.), methyl trans-4-(p-tolylsulfonyloxymethyl)cyclohexanecarboxylate (518.99 mg, 1.59 mmol, 1.5 eq.) and Cs₂CO₃ (690.74 mg, 2.12 mmol, 2 eq.) in DMF (2 ml) was stirred at 80° C. for 12 hrs. The reaction mixture was filtered, diluted with water (10 ml), extracted with EA (10 ml×3), the combined organic layers were washed with brine (20 ml×2), dried over Na₂SO₄, filtered, concentrated under reduced pressure and purified by flash silica gel chromatography (eluent: petroleum ether and EA, gradient: 0-35% EA) and prep-TLC (SiO₂, petroleum ether/EA=1/2) to give the title compound (250 mg, 730.07 μmol, 69% yield) as a yellow oil.

¹H NMR (400 MHz, CDCl₃): δ ppm 8.31 (d, J=2.4 Hz, 1H), 7.22 (d, J=1.8 Hz, 1H), 7.17 (d, J=3.1 Hz, 1H), 6.63 (d, J=2.8 Hz, 1H), 3.96-3.85 (m, 3H), 3.66 (s, 2H), 3.50 (s, 2H), 2.28-2.21 (m, 1H), 2.00 (br d, J=11.0 Hz, 3H), 1.92-1.78 (m, 2H), 1.77-1.69 (m, 2H), 1.47-1.35 (m, 3H), 1.08-1.05 (m, 2H), 0.79-0.73 (m, 2H).

STEP 2: Synthesis of trans-4-[[6-(1-methylcyclopropoxy)pyrrolo[3,2-b]pyridin-1-yl]methyl]cyclohexanecarboxylic acid—Compound (Iia) in Scheme 1—in a similar way as performed in Example 3.28, but using methyl trans-4-[[6-(1-methylcyclopropoxy)pyrrolo[3,2-b]pyridin-1-yl]methyl]cyclohexanecarboxylate instead of methyl trans-4-[(5-methyloxy-pyrrolo[3,2-b]pyridin-1-yl)methyl]cyclohexanecarboxylate

STEP 3: Synthesis of trans-3-fluoro-5-[(3S)-2-[4-[[6-(1-methylcyclopropoxy)pyrrolo[3,2-b]pyridin-1-yl]methyl]cyclohexanecarbonyl]isoxazolidin-3-yl]benzonitrile (Compound (184)) is performed as in STEP 5c of Example 1.4, but starting from trans-4-[[6-(1-methylcyclopropoxy)pyrrolo[3,2-b]pyridin-1-yl]methyl]cyclohexanecarboxylic acid instead of trans-4-[(5-cyano-6-fluoro-benzimidazol-1-yl)methyl]cyclohexanecarboxylic acid.

### Example 7: Synthesis of Compounds (272), (273), (274), (267), (268), (269), (270), (271), (275), (276), (277), (278), (286), (287)

### Example 7.1: Synthesis of Compounds (272), (273) and (274)

STEP 1: Synthesis of methyl 4-[(3-carbamoyl-4-methyl-phenyl)methyl]bicyclo[2.2.2]octane-1-carboxylate—Compound (IIIb) in Scheme 3

### Step a: Synthesis of methyl 4-(hydroxymethyl)bicyclo[2.2.2]octane-1-carboxylate

To a stirred solution of 4-methoxycarbonylbicyclo[2.2.2]octane-1-carboxylic acid (2 g, 9.42 mmol, 1 eq.) in THF (20 ml) BH₃·Me₂S (10 M, 2.83 ml, 3 eq.) was added at 0° C. under N₂ atmosphere. The reaction mixture was warmed to 20° C. and stirred for 4 hrs. The reaction mixture was quenched with MeOH (30 ml) at 20° C., stirred at 20° C. for 2 hrs, concentrated under reduced pressure and purified by column chromatography (SiO₂, petroleum ether/EA=10/1 to 5/1) to give the title compound (1.52 g, 7.67 mmol, 81% yield) as a colorless oil.

¹H NMR (400 MHz, DMSO-d₆): δ ppm 4.36 (t, J=5.4 Hz, 1H), 3.55 (s, 3H), 3.02 (d, J=5.4 Hz, 2H), 1.75-1.61 (m, 6H), 1.38-1.30 (m, 6H).

### Step b: Synthesis of Methyl 4-(bromomethyl)bicyclo[2.2.2]octane-1-carboxylate

To a solution of methyl 4-(hydroxymethyl)bicyclo[2.2.2]octane-1-carboxylate (500 mg, 2.52 mmol, 1 eq.) in DCM (3 ml) CBr₄ (2.09 g, 6.30 mmol, 2.5 eq.) and Net₃ (637.99 mg, 6.30 mmol, 877.56 μl, 2.5 eq.) were added at 0° C. Then PPh₃ (1.59 g, 6.05 mmol, 2.4 eq.) in DCM (3 ml) was added at 0° C. The mixture was stirred at 45° C. for 12 hrs, concentrated under reduced pressure and purified by column chromatography (SiO₂, petroleum ether/EA=10/1 to 5/1) to give the title compound (453 mg, 1.73 mmol, 69% yield) as a yellow oil.

¹H NMR (400 MHz, CDCl₃): δ ppm 3.65 (s, 3H), 3.21 (s, 2H), 1.88-1.75 (m, 6H), 1.58-1.47 (m, 6H).

### Step c: Synthesis of 5-bromo-2-methyl-benzamide

To a mixture of 5-bromo-2-methyl-benzoic acid (3 g, 13.95 mmol, 1 eq.) and HATU (7.96 g, 20.93 mmol, 1.5 eq.) in DMF (30 ml) DIPEA (3.61 g, 27.90 mmol, 4.86 ml, 2 eq.) was added, stirred for 10 minutes, NH₃·H₂O (9.75 g, 77.90 mmol, 10.71 ml, 28% purity, 5.58 eq.) was added and stirred at 25° C. for 16 hrs. The mixture was poured in water (200 ml), cooled to 0° C. for 20 minutes, washed with 1 N HCl (150 ml×2), saturated sodium bicarbonate solution (150 ml×2) and EA (200 ml×3), dried over Na₂SO₄, filtered, concentrated under reduced pressure and triturated with petroleum ether/EA=5/1 (20 ml) at 20° C. for 10 min to give the title compound (2.4 g, 11.19 mmol, 80% yield) as a white solid.

LC/MS: m/z 214.3 [M+H]⁺ (Method B)

¹H NMR (400 MHz, DMSO-d₆): δ ppm 7.95 (s, 1H), 7.83 (br s, 1H), 7.54-7.41 (m, 3H), 7.26-7.12 (m, 1H), 2.31 (s, 3H).

### Step d: Synthesis of methyl 4-[(3-carbamoyl-4-methyl-phenyl)methyl]bicyclo[2.2.2]octane-1-carboxylate

To a 40 ml vial equipped with a stir bar was added methyl 4-(bromomethyl)bicyclo-[2.2.2]octane-1-carboxylate (500 mg, 1.91 mmol, 1 eq.), 5-bromo-2-methyl-benzamide (532.78 mg, 2.49 mmol, 1.3 eq.), TTMSS (476.08 mg, 1.91 mmol, 590.67 μl, 1 eq.), Ir[dF(CF₃)ppy]₂(dtbpy)(PF₆) (21.48 mg, 19.15 μmol, 0.01 eq.), NiCl₂·dtbbpy (3.81 mg, 9.57 μmol, 0.005 eq.) and Na₂CO₃ (405.85 mg, 3.83 mmol, 2 eq.) in DME (20 ml). The vial was placed under nitrogen and sealed (2 batches with same amount of reagents in parallel). The reaction was stirred and irradiated with a 34 W blue LED lamp (7 cm distance), with cooling fan to keep the reaction temperature at 20° C. for 14 hrs. The reaction mixture was filtered, concentrated under reduced pressure and purified by reversed-phase HPLC (column: 80 g Flash Column Welch Ultimate XB C18 20-40 μm, 120 Å, mobile phase: water (0.1% formic acid) and ACN, gradient: 5%-50% ACN in 20 min, 50% ACN for 5 min, flow: 85 ml/min) to give the title compound out of two batches (252 mg, 798.89 μmol, 21% yield) as a white solid.

LC/MS: m/z 316.4 [M+H]⁺ (Method B) [

¹H NMR (400 MHz, CDCl₃): δ ppm 7.62 (s, 1H), 7.31 (s, 1H), 7.10 (d, J=7.8 Hz, 1H), 7.05-6.95 (m, 2H), 3.53 (s, 3H), 2.37 (s, 2H), 2.33-2.28 (m, 3H), 1.71-1.57 (m, 6H), 1.43-1.28 (m, 6H).

STEP 2: Synthesis of 4-[(3-carbamoyl-4-methyl-phenyl)methyl]bicyclo[2.2.2]octane-1-carboxylic acid—Compound (Iia) in Scheme 1—in a similar way as performed in Example 3.28, but using methyl 4-[(3-carbamoyl-4-methyl-phenyl)methyl]bicyclo[2.2.2]octane-1-carboxylate instead of methyl trans-4-[(5-methyloxy-pyrrolo[3,2-b]pyridin-1-yl)methyl]cyclohexanecarboxylate

STEP 3a: Synthesis of 5-[[4-[(3S)-3-(3-cyano-5-fluoro-phenyl)isoxazolidine-2-carbonyl]-1-bicyclo[2.2.2]octanyl]methyl]-2-methyl-benzamide (Compound (274)) is performed as in STEP 5c of Example 1.4, but starting from 4-[(3-carbamoyl-4-methyl-phenyl)methyl]bicyclo[2.2.2]octane-1-carboxylic acid instead of trans-4-[(5-cyano-6-fluoro-benzimidazol-1-yl)methyl]cyclohexanecarboxylic acid.

STEP 3b: Synthesis of 2-methyl-5-[[4-[(3S)-3-(6-methyl-3-pyridyl)isoxazolidine-2-carbonyl]-1-bicyclo[2.2.2]octanyl]methyl]benzamide (Compound (272)) is performed as in STEP 3a here-above but starting from (3S)-3-(6-methyl-3-pyridyl)isoxazolidine instead of 3-fluoro-5-[(3S)-isoxazolidin-3-yl]benzonitrile hydrochloride.

STEP 3c: Synthesis of 5-[[4-[(3S)-3-(3,5-difluorophenyl)-1,2-oxazolidine-2-carbonyl]-1-bicyclo[2.2.2]octanyl]methyl]-2-methylbenzamide (Compound (273)) is performed as in STEP 3a here-above but starting from (3S)-3-(3,5-difluorophenyl)-isoxazolidine instead of 3-fluoro-5-[(3S)-isoxazolidin-3-yl]benzonitrile hydrochloride.

### Example 7.2: Synthesis of Compounds (269), (270) and (271)

STEP 1: Synthesis of methyl 4-[(3-carbamoyl-4-methyl-phenyl)methyl]norbornane-1-carboxylate—Compound (IIIb) in Scheme 3 in a similar way as performed in Example 7.1, starting from 5-bromo-2-methyl-benzamide

STEP 2: Synthesis of 4-[(3-carbamoyl-4-methyl-phenyl)methyl]norbornane-1-carboxylic acid—Compound (Iia) in Scheme 1—in a similar way as performed in Example 1.1, but using methyl 4-[(3-carbamoyl-4-methyl-phenyl)methyl]norbornane-1-carboxylate instead of trans-4-(benzimidazol-1-ylmethyl)cyclohexanecarboxylate, and implementing the saponification with NaOH instead of LiOH.

STEP 3a: Synthesis of 5-[[4-[(3S)-3-(3-cyano-5-fluoro-phenyl)isoxazolidine-2-carbonyl]norbornan-1-yl]methyl]-2-methyl-benzamide (Compound (270)) is performed as in STEP 5c of Example 1.4, but starting from 4-[(3-carbamoyl-4-methyl-phenyl)methyl]norbornane-1-carboxylic acid instead of trans-4-[(5-cyano-6-fluoro-benzimidazol-1-yl)methyl]cyclohexanecarboxylic acid.

STEP 3b: Synthesis of 5-[[4-[(3S)-3-(3,5-difluorophenyl)isoxazolidine-2-carbonyl]norbornan-1-yl]methyl]-2-methyl-benzamide (Compound (269)) is performed as in STEP 3a here-above but starting from (3S)-3-(3,5-difluorophenyl)-1,2-oxazolidine instead of 3-fluoro-5-[(3S)-isoxazolidin-3-yl]benzonitrile hydrochloride.

STEP 3c: Synthesis of 2-methyl-5-[[4-[(3S)-3-(6-methyl-3-pyridyl)isoxazolidine-2-carbonyl]norbornan-1-yl]methyl]benzamide (Compound (271)) is performed as in STEP 3a here-above but starting from (3S)-3-(6-methyl-3-pyridyl)isoxazolidine instead of 3-fluoro-5-[(3S)-isoxazolidin-3-yl]benzonitrile hydrochloride.

### Example 7.3: Synthesis of Compounds (267), (268), (286)

### STEP 1: Synthesis of 5-bromo-2-methyl-pyridine-3-carbonitrile

### Step a: Synthesis of 5-bromo-N-tert-butyl-2-methyl-pyridine-3-carboxamide

To a solution of 5-bromo-2-methyl-pyridine-3-carboxylic acid (2 g, 9.26 mmol, 1 eq.), HATU (5.28 g, 13.89 mmol, 1.5 eq.) and NetiPr₂ (3.59 g, 27.77 mmol, 4.84 ml, 3 eq.) in DMF (20 ml) 2-methylpropan-2-amine (812.51 mg, 11.11 mmol, 1.17 ml, 1.2 eq.) was added under N₂ atmosphere and stirred at 25° C. for 12 hrs. The reaction mixture was poured into water (200 ml), extracted with EA (100 ml×3), the combined organic layers were washed with brine (100 ml), dried over Na₂SO₄, filtered, concentrated, and purified by column chromatography (SiO₂, petroleum ether/EA=1/0 to 1/1) to give the title compound (2.5 g, 9.22 mmol, 100% yield) as a white solid.

¹H NMR (400 MHz, CDCl₃): δ ppm 8.58 (s, 1H), 7.75 (s, 1H), 5.65 (br s, 1H), 2.60 (s, 3H), 1.49 (s, 10H).

### Step b: Synthesis of 5-bromo-2-methyl-pyridine-3-carbonitrile

To a solution of 5-bromo-N-tert-butyl-2-methyl-pyridine-3-carboxamide (2.4 g, 8.85 mmol, 1 eq.) in toluene (20 ml) POCl₃ (13.57 g, 88.51 mmol, 8.23 ml, 10 eq.) was added under N₂ atmosphere. The reaction mixture was stirred at 110° C. for 2 hrs, poured into saturated NaHCO₃ aqueous solution (200 ml), extracted with EA (50 ml×3), the combined organic layers were washed with brine (50 ml), dried over Na₂SO₄, filtered, concentrated and purified by column chromatography (SiO₂, petroleum ether/EA=1/0 to 1/1) to give the title compound (1.2 g, 6.09 mmol, 69% yield) as a white solid.

¹H NMR (400 MHz, CDCl₃): δ ppm 8.67 (d, J=2.3 Hz, 1H), 7.94 (d, J=2.3 Hz, 1H), 2.67 (s, 3H).

STEP 2: Synthesis of methyl trans-4-[(5-cyano-6-methyl-3-pyridyl)methyl]cyclohexanecarboxylate—Compound (IIIb) in Scheme 3—in a similar way as performed in Example 7.1 step d, but using 5-cyano-6-methyl-3-bromopyridine

STEP 3: Synthesis of trans-4-[(5-cyano-6-methyl-3-pyridyl)methyl]cyclohexanecarboxylic acid—Compound (Iia) in Scheme 1—in a similar way as performed in Example 3.28, but using methyl trans-4-[(5-cyano-6-methyl-3-pyridyl)methyl]cyclohexanecarboxylate instead of methyl trans-4-[(5-methyloxy-pyrrolo[3,2-b]pyridin-1-yl)methyl]cyclohexanecarboxylate

STEP 4a: Synthesis of trans-5-[[4-[(3S)-3-(3-cyano-5-fluoro-phenyl)isoxazolidine-2-carbonyl]cyclohexyl]methyl]-2-methyl-pyridine-3-carbonitrile (Compound (268)) is performed as in STEP 5c of Example 1.4, but starting from trans-4-[(5-cyano-6-methyl-3-pyridyl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-5-[[4-[(3S)-3-(3,5-difluorophenyl)isoxazolidine-2-carbonyl]cyclohexyl]methyl]-2-methyl-pyridine-3-carbonitrile (Compound (267)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

STEP 4c: Synthesis of trans-5-[[4-[(3S)-3-(3-cyano-5-fluoro-4-methyl-phenyl)isoxazolidine-2-carbonyl]cyclohexyl]methyl]-2-methyl-pyridine-3-carbonitrile (Compound (286)) is performed as in STEP 4a here-above but starting from (3S)-3-(3-cyano-5-fluoro-4-methyl-phenyl)isoxazolidine instead of 3-fluoro-5-[(3S)-isoxazolidin-3-yl]benzonitrile hydrochloride.

### Example 7.4: Synthesis of Compounds (275), (276), (287)

### STEP 1: Synthesis of 5-bromo-4-methyl-pyridine-3-carbonitrile

To a solution of 3,5-dibromo-4-methyl-pyridine (10 g, 39.85 mmol, 1 eq.) in DMF (300 ml) was added CuCN (3.57 g, 39.85 mmol, 1 eq.) and the mixture was stirred at 140° C. for 5 hrs. The reaction mixture was filtered, diluted with water (1000 ml), extracted with EA (200 ml×3), the combined organic layers were washed with brine (200 ml), dried over Na₂SO₄, filtered, concentrated under reduced pressure and purified by column chromatography (SiO₂, petroleum ether/EA=4/1 to 3/1) to give the title compound (2.96 g, 14.87 mmol, 37% yield) as a white solid.

LC/MS: m/z 197.0 [M+H]⁺ (Method B)

¹H NMR (400 MHz, CDCl₃): δ ppm 8.85 (s, 1H), 8.73 (s, 1H), 2.67 (s, 3H).

STEP 2: Synthesis of methyl trans-4-[(5-cyano-4-methyl-3-pyridyl)methyl]cyclohexanecarboxylate—Compound (IIIb) in Scheme 3—in a similar way as performed in Example 7.1 step d, but using 5-cyano-4-methyl-3-bromopyridine

STEP 3: Synthesis of trans-4-[(5-cyano-4-methyl-3-pyridyl)methyl]cyclohexanecarboxylic acid—Compound (Iia) in Scheme 1—in a similar way as performed in Example 3.28, but using methyl trans-4-[(5-cyano-4-methyl-3-pyridyl)methyl]cyclohexanecarboxylate instead of methyl trans-4-[(5-methyloxy-pyrrolo[3,2-b]pyridin-1-yl)methyl]cyclohexanecarboxylate

STEP 4a: Synthesis of trans-5-[[4-[(3S)-3-(3-cyano-5-fluoro-phenyl)isoxazolidine-2-carbonyl]cyclohexyl]methyl]-4-methyl-pyridine-3-carbonitrile (Compound (276)) is performed as in STEP 5c of Example 1.4, but starting from trans-4-[(5-cyano-4-methyl-3-pyridyl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-5-[[4-[(3S)-3-(3,5-difluorophenyl)isoxazolidine-2-carbonyl]cyclohexyl]methyl]-4-methyl-pyridine-3-carbonitrile (Compound (275)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

STEP 4c: Synthesis of trans-5-[[4-[(3S)-3-(3-cyano-5-fluoro-4-methyl-phenyl)isoxazolidine-2-carbonyl]cyclohexyl]methyl]-4-methyl-pyridine-3-carbonitrile (Compound (287)) is performed as in STEP 4a here-above but starting from (3S)-3-(3-cyano-5-fluoro-4-methyl-phenyl)isoxazolidine instead of 3-fluoro-5-[(3S)-isoxazolidin-3-yl]benzonitrile hydrochloride.

### Example 7.5: Synthesis of Compounds (277), (278)

STEP 1: Synthesis of 5-bromo-6-methyl-pyridine-3-carbonitrile—in a similar way as performed in Example 7.4, but using 3,5-dibromo-6-methyl-pyridine

STEP 2: Synthesis of methyl trans-4-[(5-cyano-2-methyl-3-pyridyl)methyl]cyclohexanecarboxylate—Compound (IIIb) in Scheme 3—in a similar way as performed in Example 7.1 step d, but using 5-cyano-2-methyl-3-bromopyridine

STEP 3: Synthesis of trans-4-[(5-cyano-2-methyl-3-pyridyl)methyl]cyclohexanecarboxylic acid—Compound (Iia) in Scheme 1—in a similar way as performed in Example 3.28, but using methyl trans-4-[(5-cyano-2-methyl-3-pyridyl)methyl]cyclohexanecarboxylate instead of methyl trans-4-[(5-methyloxy-pyrrolo[3,2-b]pyridin-1-yl)methyl]cyclohexanecarboxylate

STEP 4a: Synthesis of trans-5-[[4-[(3S)-3-(3-cyano-5-fluoro-phenyl)isoxazolidine-2-carbonyl]cyclohexyl]methyl]-6-methyl-pyridine-3-carbonitrile (Compound (277)) is performed as in STEP 5c of Example 1.4, but starting from trans-4-[(5-cyano-2-methyl-3-pyridyl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-5-[[4-[(3S)-3-(3,5-difluorophenyl)isoxazolidine-2-carbonyl]cyclohexyl]methyl]-6-methyl-pyridine-3-carbonitrile formic acid salt (Compound (278)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

### Example 7.6: Synthesis of Compounds (310), (315)

### STEP 1: Synthesis of 6-chloro-2-methyl-[1,2,4]triazolo[1,5-b]pyridazine

### Step a: Synthesis of N′-(6-chloropyrdazin-3-yl)-N,N-dimethyl-acetamidine

To a solution of 6-chloropyridazin-3-amine (5 g, 38.60 mmol, 1 eq.) in DMF (50 ml) was added 1,1-dimethoxy-N,N-dimethyl-ethanamine (10.28 g, 77.19 mmol, 11.29 ml, 2 eq.). The mixture was stirred at 130° C. for 3 hrs. The residue was concentrated under reduced pressure to give 7.67 g (38.61 mmol, quant. Yield) of the title compound as a white solid.

LC/MS: m/z 199.2 [M+H]⁺, Rt=0.123 min (LC/MS method C).

### Step b: Synthesis of N′-(6-chloropyridazin-3-yl)-N-hydroxy-acetamidine

To a solution of N′-(6-chloropyridazin-3-yl)-N,N-dimethyl-acetamidine (7.67 g, 38.61 mmol, 1 eq.) in i-PrOH (80 ml) was added NH₂OH HCl (3.18 g, 45.81 mmol, 1.3 eq.). The mixture was stirred at 50° C. for 3 hrs. The residue was concentrated under reduced pressure to give 6.58 g (35.26 mmol, 91% yield) of the title compound as a white solid.

LC/MS: m/z 187.2 [M+H]⁺, Rt=0.181 min (LC/MS method C)

¹H NMR (CDCl₃, 400 MHz) δ ppm 7.38 (d, J=9.3 Hz, 1H), 6.97 (d, J=9.3 Hz, 1H), 2.44 (s, 3H).

### Step c: Synthesis of 6-chloro-2-methyl-[1,2,4]triazolo[1,5-b]pyridazine

To a solution of N′-(6-chloropyridazin-3-yl)-N-hydroxy-acetamidine (6.58 g, 35.26 mmol, 1 eq.) in THF (70 ml) was added TFAA (8.15 g, 38.79 mmol, 5.40 ml, 1.1 eq.) at 0° C. The mixture was stirred at 25° C. for 3 hrs. The residue was diluted with water (100 ml) and extracted with EA (100 ml×2). The combined organic layers were dried over Na₂SO₄, filtered, concentrated under reduced pressure and purified by flash silica gel chromatography (ISCO®; 8 g SepaFlash® Silica Flash Column, eluents: EA and petroleum ether, gradient: 0˜60% EA; flow: 80 ml/min) to give 5.1 g (30.25 mmol, 86% yield) of the title compound as a white solid.

LC/MS: m/z 169.1 [M+H]⁺, Rt=0.266 min (LC/MS method B)

¹H NMR (CDCl₃, 400 MHz): δ ppm 7.99 (d, J=9.3 Hz, 1H), 7.39 (d, J=9.3 Hz, 1H), 2.66 (s, 3H).

### STEP 2: Synthesis of Methyl trans-4-[(2-methyl-[1,2,4]triazolo[1,5-b]pyridazin-6-yl)methyl]cyclohexanecarboxylate—Compound (IIIb) in Scheme 3

To a 40 mL vial equipped with a stir bar was added 6-chloro-2-methyl-[1,2,4]triazolo[1,5-b]pyridazine (500 mg, 2.97 mmol, 1 eq), methyl (1r,4r)-4-(bromomethyl)cyclohexane-1-carboxylate (906.53 mg, 3.86 mmol, 1.3 eq), NiCl₂·dtbbpy (5.90 mg, 14.83 μmol, 0.005 eq), TTMSS (737.50 mg, 2.97 mmol, 915.01 uL, 1 eq), Ir[dF(CF₃)ppy]₂(dtbpy)(PF₆) (33.27 mg, 29.66 μmol, 0.01 eq), Na₂CO₃ (628.71 mg, 5.93 mmol, 2 eq) in DME (30 mL). The vial was sealed and placed under nitrogen was added. The reaction was stirred and irradiated with a 34 W blue LED lamp (7 cm away), with cooling fan to keep the reaction temperature at 25° C. for 14 hr. The reaction mixture was filtered and concentrated under reduced pressure, the crude product was purified by reversed-phase HPLC (0.1% FA condition: flow: 100 mL/min; gradient from 100% H₂O (0.1% FA)/0% ACN to 50% H₂O (0.1% FA)/50% ACN in 20 min; 50% H₂O (0.1% FA)/50% ACN in 20 min; column: Welch Ultimate XB_C18, 20-40 μm, 120 A) to give methyl trans-4-[(2-methyl-[1,2,4]triazolo[1,5-b]pyridazin-6-yl)methyl]cyclohexanecarboxylate (0.2 g, 547.96 μmol, 4.62% yield, 79% purity) as a white solid.

STEP 3: Synthesis of trans-4-[(2-methyl-[1,2,4]triazolo[1,5-b]pyridazin-6-yl)methyl]cyclohexanecarboxylic acid—Compound (Iia) in Scheme 1—in a similar way as performed in Example 3.28, but using trans-4-[(2-methyl-[1,2,4]triazolo[1,5-b]pyridazin-6-yl)methyl]cyclohexanecarboxylate instead of methyl trans-4-[(5-methyloxy-pyrrolo[3,2-b]pyridin-1-yl)methyl]cyclohexanecarboxylate

To a solution of methyl trans-4-[(2-methyl-[1,2,4]triazolo[1,5-b]pyridazin-6-yl)methyl]cyclohexanecarboxylate (1.9 g, 6.59 mmol, 1 eq) in THF (10 mL) was added LiOH H₂O (553.03 mg, 13.18 mmol, 2 eq) and H₂O (10 mL). The mixture was stirred at 25° C. for 2 hr. The pH was adjusted to 3 with 1M HCl. Trans-4-[(2-methyl-[1,2,4]triazolo[1,5-b]pyridazin-6-yl)methyl]cyclohexanecarboxylic acid (1.61 g, 5.75 mmol, 87.29% yield, 98% purity) was obtained as a white solid.

LCMS: m/z 275.3 [M+1]+

¹H NMR: (DMSO-d₆, 400 MHz) δ ppm 8.20 (d, J=9.1 Hz, 1H), 7.52 (d, J=9.3 Hz, 1H), 2.75 (d, J=6.9 Hz, 2H), 2.49-2.49 (m, 3H), 2.13 (tt, J=3.3, 12.0 Hz, 1H), 1.94-1.83 (m, 2H), 1.78-1.72 (m, 1H), 1.68 (br d, J=12.9 Hz, 2H), 1.33-1.23 (m, 2H), 1.12-0.99 (m, 2H)

STEP 4a: Synthesis of trans-[(3S)-3-(3,4-difluorophenyl)isoxazolidin-2-yl]-[4-[(2-methyl-[1,2,4]triazolo[1,5-b]pyridazin-6-yl)methyl]cyclohexyl]methanone (Compound (310)) is performed as in STEP 5c of Example 1.4, but starting from trans-4-[(2-methyl-[1,2,4]triazolo[1,5-b]pyridazin-6-yl)methyl]cyclohexanecarboxylic acid and (3S)-3-(3,4-difluorophenyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

STEP 4b: Synthesis of [(3S)-3-(3,5-difluorophenyl)-1,2-oxazolidin-2-yl]-[4-[(2-methyl-[1,2,4]triazolo[1,5-b]pyridazin-6-yl)methyl]cyclohexyl]methanone (Compound (315)).

To a solution of trans-4-[(2-methyl-[1,2,4]triazolo[1,5-b]pyridazin-6-yl)methyl]cyclohexanecarboxylic acid (100 mg, 364.54 μmol, 1 eq) in DMF (1 mL) was added DIEA (235.57 mg, 1.82 mmol, 317.48 uL, 5 eq) and HATU (207.91 mg, 546.81 μmol, 1.5 eq), (3S)-3-(3,5-difluorophenyl)-1,2-oxazolidine (82.08 mg, 437.45 μmol, 1.2 eq, HCl). The mixture was stirred at 25° C. for 12 hr. The residue was diluted with H₂O 10 mL and extracted with ethyl acetate 10 mL (10 mL*2). The combined organic layers were dried over Na₂SO₄, filtered, and concentrated under reduced pressure to give a residue. The residue was purified by prep-HPLC (column: Phenomenex C18 75*30 mm*3 μm; mobile phase: [water (0.225% FA)-ACN]; B %: 25%-55%, 7 min) to give compound (315) (86 mg, 208.95 μmol, 57.32% yield, 99% purity) was obtained as a white solid.

### Example 8: Synthesis of Compounds (1), (109), (124), (125), (135), (136), (139), (140), (141), (142), (143), (147), (148), (160), (161), (162), (163), (164), (165), (167), (168), (169), (170), (171), (172), (173), (174), (175), (176), (177), (178), (179), (180), (181), (182), (183), (197), (205), (206), (207), (208), (209), (210), (212), (213), (216), (219), (220), (221), (222), (223), (225), (226), (227), (237), (238), (239), (240), (242), (243), (244), (245), (246), (247), (248), (249), (250), (256), (257), (256), (260), (261), (262), (263), (264), (265), (266), (279), (280), (281), (282), (283), (284), (285), (289) (292), (293), (294), (295), (296), (297), (303), (304), (306), (307), (311), (316), (321), 372, 373, 374 Via Photoredox Catalysis or Cross Electrophile-Coupling

### Synthesis of Methyl trans-4-(bromomethyl)cyclohexanecarboxylate—Compound (Ivb) of Scheme 3

To a stirred solution at 0° C. of methyl 4-(hydroxymethyl)cyclohexanecarboxylate (4.0 g, 23.0 mmol) and triphenylphosphane (9.10 g, 35.0 mmol) in CH₂Cl₂ (172 ml) NBS (8.30 g, 46.0 mmol) was added in portions. The dark solution was stirred at rt for 10 min, volatile components were removed under reduced pressure and the resulting dark residue is triturated for 1 hour with heptane/EA (1/1; 200 ml). The suspension is decanted, and the solution evaporated to give a yellow solid, which was triturated with heptane (200 ml). The solution was evaporated. And the resulting crude product (5 g) purified by column chromatography (80 g, SiO₂; EA/heptane gradient 0-30% EA) to give the title compound (3.86 g, 16.4 mmol, 71% yield).

¹H NMR (400 MHz, DMSO-d₆): δ ppm 3.58 (s, 3H), 3.42 (d, 2H), 2.23 (m, 1H), 1.88 (m, 4H), 1.57 (m, 1H), 1.33 (m, 2H), 1.05 (m, 2H).

### Example 8.1: Synthesis of Compounds (109), (139)

### STEP 1: Synthesis of methyl trans-4-[(3-cyano-4-methyl-phenyl)methyl]cyclohexanecarboxylate (Typical Example of Photoredox Catalysis)—Compound of Formula (IIIb) in Scheme 3

In a vial under an Ar atmosphere NiCl₂ ethylene glycol dimethyl ether complex (11.2 mg, 51 μmol) and 4,4′-di-tert-butyl-2,2′-bipyridine (13.7 mg, 51 μmol) were suspended in 1.6 ml THF. The suspension was sonicated at 50° C. in an ultrasound bath for 10 min. Volatile components were removed under reduced pressure, [Ir(dF(Me)ppy)₂(dtbbpy)]PF₆ (10.3 mg, 10.2 μmol) was added and suspended in DME (10 ml) to form a catalyst suspension. 5-bromo-2-methyl-benzonitrile (200 mg, 1.02 mmol), methyl trans-4-(bromomethyl)cyclo-hexanecarboxylate (360 mg, 1.50 mmol), LiOH (48.9 mg, 2.04 mmol) and tris(trimethyl-silyl)silane (254 mg, 1.00 mmol, 0.315 ml) were placed in a vial under an Ar atmosphere. The catalyst suspension was added and Ar was bubbled through the resulting suspension for 10 min. The vial was sealed and irradiated in a PennOC photoreactor M1 (100% light intensity) for 24 hrs.

The reaction mixture was diluted with EA and water and the aqueous layer extracted with EA. The combined organic layers were dried over Na₂SO₄, filtered and concentrated to afford the crude title compound, which was purified by column chromatography (SiO₂; EA/heptane gradient) (120 mg, 450 μmol, 44% yield).

¹H NMR (400 MHz, DMSO-d₆): δ ppm 7.40 (s, 1H), 7.23 (m, 2H), 3.42 (s, 3H), 2.36 (m, 2H), 2.34 (s, 3H), 2.07 (m, 1H), 1.73 (m, 3H), 1.47 (m, 2H), 1.33 (m, 1H), 1.11 (m, 2H), 0.81 (m, 2H).

STEP 2: Synthesis of trans-4-[(3-cyano-4-methyl-phenyl)methyl]cyclohexanecarboxylic acid—Compound (Iia) in Scheme 1—in a similar way as performed in Example 1.1, but using methyl trans-4-[(3-cyano-4-methyl-phenyl)methyl]cyclohexanecarboxylate instead of trans-4-(benzimidazol-1-ylmethyl)cyclohexanecarboxylate

STEP 3a: Synthesis of trans-5-((4-((S)-3-(3-cyano-5-fluorophenyl)isoxazolidine-2-carbonyl)cyclohexyl)methyl)-2-methylbenzonitrile (Compound (109)) is performed as in STEP 5c of Example 1.4, but starting from trans-4-[(3-cyano-4-methyl-phenyl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-2-methyl-5-[[4-[(3S)-3-pyrazin-2-ylisoxazolidine-2-carbonyl]cyclohexyl]methyl]benzonitrile (Compound (139)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.2: Synthesis of Compounds (225), (226), (227)

STEP 1: Synthesis of methyl trans-4-[(2-methylimidazo[1,2-a]pyridin-6-yl)methyl]cyclohexanecarboxylate (typical example of photoredox catalysis as detailed in JACS 2016, 138, 8084-8087)—Compound of formula (IIIb) in Scheme 3

In a vial under an Ar atmosphere NiBr₂ ethylene glycol dimethyl ether complex (15.0 mg, 50 μmol) and 4,4′-di-tert-butyl-2,2′-bipyridine (13.0 mg, 50 μmol) were suspended in 9 ml dioxane. The suspension was sonicated at 50° C. in an ultrasound bath for 10 min to form a catalyst suspension.

6-bromo-2-methyl-imidazo[1,2-a]pyridine (210 mg, 1.00 mmol), methyl trans-4-(bromo-methyl)cyclohexanecarboxylate (350 mg, 1.50 mmol), LiOH (48.0 mg, 2.00 mmol), tris(trimethylsilyl)silane (300 mg, 1.20 mmol, 0.37 ml) and [Ir(dF(Me)ppy)₂(dtbbpy)]PF₆ (20 mg, 20.0 μmol) were placed in a vial under an Ar atmosphere. The catalyst suspension was added and Ar was bubbled through the resulting suspension for 10 min. The vial was sealed and irradiated in a PennOC photoreactor M1 (100% light intensity) for 24 hrs. Volatile components were removed under reduced pressure and the resulting residue was partitioned between EA and water. The aqueous layer extracted with EA and the combined organic layers were dried over Na₂SO₄, filtered and concentrated to afford the crude title compound, which was purified by column chromatography (SiO₂; EA/MeOH gradient; 0-20% MeOH) (60 mg, 210 μmol, 21% yield).

STEP 2: Synthesis of trans-4-[(2-methylimidazo[1,2-a]pyridin-6-yl)methyl]cyclohexanecarboxylic acid—Compound (Iia) in Scheme 1—in a similar way as performed in Example 1.1, but using methyl trans-4-[(2-methylimidazo[1,2-a]pyridin-6-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(2-methylimidazo[1,2-a]pyridin-6-yl)methyl]cyclohexanecarbonyl]isoxazolidin-3-yl]benzonitrile (Compound (225)) is performed as in STEP 5c of Example 1.4, but starting from trans-4-[(2-methylimidazo[1,2-a]pyridin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(2-methylimidazo[1,2-a]pyridin-6-yl)methyl]cyclohexyl]-[(3S)-3-pyrazin-2-ylisoxazolidin-2-yl]methanone (Compound (226)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-[(3S)-3-(3,5-difluorophenyl)isoxazolidin-2-yl]-[4-[(2-methylimidazo[1,2-a]pyridin-6-yl)methyl]cyclohexyl]methanone (Compound (227)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 8.3: Synthesis of Compounds (140), (141)

STEP 1: Synthesis of methyl trans-4-[(5-cyano-3-pyridyl)methyl]cyclohexanecarboxylate—Compound of formula (IIIb) in Scheme 3, is performed as in example 8.2 here-above

STEP 2: Synthesis of trans-4-[(5-cyano-3-pyridyl)methyl]cyclohexanecarboxylic acid—Compound (Iia) in Scheme 1—in a similar way as performed in Example 1.1, but using methyl trans-4-[(5-cyano-3-pyridyl)methyl]cyclohexanecarboxylate instead of trans-4-(benzimidazol-1-ylmethyl)cyclohexanecarboxylate

STEP 3a: Synthesis of trans-5-[[4-[(3S)-3-(3-cyano-5-fluoro-phenyl)isoxazolidine-2-carbonyl]cyclohexyl]methyl]pyridine-3-carbonitrile (Compound (141)) is performed as in STEP 5c of Example 1.4, but starting from trans-4-[(5-cyano-3-pyridyl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-5-[[4-[(3S)-3-pyrazin-2-ylisoxazolidine-2-carbonyl]cyclohexyl]methyl]pyridine-3-carbonitrile (Compound (140)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.4: Synthesis of Compounds (135), (136)

STEP 1: Synthesis of methyl trans-4-[[6-(2-methylimidazol-1-yl)pyrimidin-4-yl]methyl]cyclohexanecarboxylate—Compound of formula (IIIb) in Scheme 3, is performed in a similar way as in example 8.2 here-above

STEP 2: Synthesis of trans-4-[[6-(2-methylimidazol-1-yl)pyrimidin-4-yl]methyl]cyclohexanecarboxylic acid—Compound (Iia) in Scheme 1—in a similar way as performed in Example 1.1, but using methyl trans-4-[[6-(2-methylimidazol-1-yl)pyrimidin-4-yl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[6-(2-methylimidazol-1-yl)pyrimidin-4-yl]methyl]cyclohexanecarbonyl]isoxazolidin-3-yl]benzonitrile (Compound (135)) is performed as in STEP 5c of Example 1.4, but starting from trans-4-[[6-(2-methylimidazol-1-yl)pyrimidin-4-yl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[[6-(2-methylimidazol-1-yl)pyrimidin-4-yl]methyl]cyclohexyl]-[(3S)-3-(p-tolyl)isoxazolidin-2-yl]methanone (Compound (136)) is performed as in STEP 3a here-above but starting from (3S)-3-(p-tolyl)isoxazolidine instead of 3-fluoro-5-[(3S)-isoxazolidin-3-yl]benzonitrile hydrochloride.

### Example 8.5: Synthesis of Compounds (124), (125)

STEP 1: Synthesis of methyl trans-4-[[6-(3,5-dimethyl pyrazol-1-yl)pyrimidin-4-yl]methyl]cyclohexanecarboxylate—Compound of formula (IIIb) in Scheme 3, is performed in a similar way as in example 8.2 here-above

STEP 2: Synthesis of trans-4-[[6-(3,5-dimethylpyrazol-1-yl)pyrimidin-4-yl]methyl]cyclohexanecarboxylic acid—Compound (Iia) in Scheme 1—in a similar way as performed in Example 1.1, but using methyl trans-4-[[6-(3,5-dimethyl pyrazol-1-yl)pyrimidin-4-yl]methyl]cyclohexanecarboxylate instead of trans-4-(benzimidazol-1-ylmethyl)cyclohexanecarboxylate

STEP 3a: Synthesis of trans-3-[(3S)-2-[4-[[6-(3,5-dimethylpyrazol-1-yl)pyrimidin-4-yl]methyl]cyclohexanecarbonyl]isoxazolidin-3-yl]-5-fluoro-benzonitrile (Compound (124)) is performed as in STEP 5c of Example 1.4, but starting from trans-4-[[6-(3,5-dimethylpyrazol-1-yl)pyrimidin-4-yl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[[6-(3,5-dimethylpyrazol-1-yl)pyrimidin-4-yl]methyl]cyclohexyl]methanone (125)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 8.6: Synthesis of Compounds (142), (143), (337) and 338

STEP 1: Synthesis of methyl trans-4-([1,2,4]triazolo[1,5-a]pyridin-6-ylmethyl)cyclohexanecarboxylate—Compound of formula (IIIb) in Scheme 3, is performed in a similar way as in example 8.2 here-above

STEP 2: Synthesis of trans-4-([1,2,4]triazolo[1,5-a]pyridin-6-ylmethyl)cyclohexanecarboxylic acid—Compound (Iia) in Scheme 1—in a similar way as performed in Example 1.1, but using methyl trans-4-([1,2,4]triazolo[1,5-a]pyridin-6-ylmethyl)cyclohexanecarboxylate instead of trans-4-(benzimidazol-1-ylmethyl)cyclohexanecarboxylate

STEP 3a: Synthesis of trans-3-fluoro-5-[(3S)-2-[4-([1,2,4]triazolo[1,5-a]pyridin-6-ylmethyl)cyclohexanecarbonyl]isoxazolidin-3-yl]benzonitrile (Compound (142)) is performed as in STEP 5c of Example 1.4, but starting from trans-4-([1,2,4]triazolo[1,5-a]pyridin-6-ylmethyl)cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-pyrazin-2-ylisoxazolidin-2-yl]-[4-([1,2,4]triazolo[1,5-a]pyridin-6-ylmethyl)cyclohexyl]methanone (Compound (143)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-[(3S)-3-(5-fluoro-6-methylpyridin-3-yl)-1,2-oxazolidin-2-yl]-[4-([1,2,4]triazolo[1,5-a]pyridin-6-ylmethyl)cyclohexyl]methanone (Compound (337)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride.

Chiral SFC: RT 1.31 min (100%, Method X)

STEP 3c: Synthesis of trans-[(3S)-3-(5-fluoropyridin-3-yl)-1,2-oxazolidin-2-yl]-[4-([1,2,4]triazolo[1,5-a]pyridin-6-ylmethyl)cyclohexyl]methanone (Compound (338)) is performed as in STEP 3a here-above but starting from 3-(5-Fluoro-3-pyridyl)isoxazolidine instead of 3-fluoro-5-[(3S)-isoxazolidin-3-yl]benzonitrile hydrochloride

Chiral SFC: RT 1.32 min (100%, Method X)

### Example 8.7: Synthesis of Compounds (147), (148)

STEP 1: Synthesis of methyl trans-4-[(5-fluoro-3-pyridyl)methyl]cyclohexanecarboxylate—Compound of formula (IIIb) in Scheme 3, is performed in a similar way as in example 8.2 here-above

STEP 2: Synthesis of trans-4-[(5-fluoro-3-pyridyl)methyl]cyclohexanecarboxylic acid—Compound (Iia) in Scheme 1—in a similar way as performed in Example 1.1, but using methyl trans-4-[(5-fluoro-3-pyridyl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(5-fluoro-3-pyridyl)methyl]cyclohexanecarbonyl]isoxazolidin-3-yl]benzonitrile (Compound (148)) is performed as in STEP 5c of Example 1.4, but starting from trans-4-[(5-fluoro-3-pyridyl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(5-fluoro-3-pyridyl)methyl]cyclohexyl]-[(3S)-3-pyrazin-2-ylisoxazolidin-2-yl]methanone (Compound (147)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.8: Synthesis of Compounds (256), (257), (259)

STEP 1: Synthesis of methyl trans-4-[[3-fluoro-5-(hydroxymethyl)phenyl]methyl]cyclohexanecarboxylate—Compound of formula (IIIb) in Scheme 3, is performed in a similar way as in example 8.2 here-above

STEP 2: Synthesis of trans-4-[[3-fluoro-5-(hydroxymethyl)phenyl]methyl]cyclohexanecarboxylic acid—Compound (Iia) in Scheme 1—in a similar way as performed in Example 1.1, but using methyl trans-4-[[3-fluoro-5-(hydroxymethyl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[3-fluoro-5-(hydroxymethyl)phenyl]methyl]cyclohexanecarbonyl]isoxazolidin-3-yl]benzonitrile (Compound (256)) is performed as in STEP 5c of Example 1.4, but starting from trans-4-[[3-fluoro-5-(hydroxymethyl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-5-[(3S)-2-[4-[[3-fluoro-5-(hydroxymethyl)phenyl]methyl]cyclohexanecarbonyl]isoxazolidin-3-yl]pyridine-3-carbonitrile (Compound (257)) is performed as in STEP 3a here-above but starting from 5-[(3S)-2-isoxazolidin-3-yl]pyridine-3-carbonitrile instead of 3-fluoro-5-[(3S)-isoxazolidin-3-yl]benzonitrile hydrochloride.

STEP 3c: Synthesis of trans-[4-[[3-fluoro-5-(hydroxymethyl)phenyl]methyl]cyclohexyl]-[(3S)-3-pyrazin-2-ylisoxazolidin-2-yl]methanone (Compound (259)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.9: Synthesis of Compounds (279), (280)

STEP 1: Synthesis of methyl trans-4-[[3-fluoro-4-(2-hydroxyethyl)phenyl]methyl]cyclohexanecarboxylate—Compound of formula (IIIb) in Scheme 3, is performed in a similar way as in example 8.2 here-above

STEP 2: Synthesis of trans-4-[[3-fluoro-4-(2-hydroxyethyl)phenyl]methyl]cyclohexanecarboxylic acid—Compound (Iia) in Scheme 1—in a similar way as performed in Example 1.1, but using methyl trans-4-[[3-fluoro-4-(2-hydroxyethyl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[3-fluoro-4-(2-hydroxyethyl)phenyl]methyl]cyclohexanecarbonyl]isoxazolidin-3-yl]benzonitrile (Compound (279)) is performed as in STEP 5c of Example 1.4, but starting from trans-4-[[3-fluoro-4-(2-hydroxyethyl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[[3-fluoro-4-(2-hydroxyethyl)phenyl]methyl]cyclohexyl]methanone (Compound (280)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 8.10: Synthesis of Compounds (282), (283)

STEP 1: Synthesis of methyl trans-4-[[3-fluoro-5-(2-hydroxyethyl)phenyl]methyl]cyclohexanecarboxylate—Compound of formula (IIIb) in Scheme 3, as in example 8.2 here-above

STEP 2: Synthesis of trans-4-[[3-fluoro-5-(2-hydroxyethyl)phenyl]methyl]cyclohexanecarboxylic acid—Compound (Iia) in Scheme 1—in a similar way as performed in Example 1.1, but using methyl trans-4-[[3-fluoro-5-(2-hydroxyethyl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[3-fluoro-5-(2-hydroxyethyl)phenyl]methyl]cyclohexanecarbonyl]isoxazolidin-3-yl]benzonitrile (Compound (282)) is performed as in STEP 5c of Example 1.4, but starting from trans-4-[[3-fluoro-5-(2-hydroxyethyl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[[3-fluoro-5-(2-hydroxyethyl)phenyl]methyl]cyclohexyl]methanone (Compound (283)) is performed as in STEP 3a here-above but starting from (3S)-3-(3,5-difluorophenyl)isoxazolidin instead of 3-fluoro-5-[(3S)-isoxazolidin-3-yl]benzonitrile hydrochloride.

### Example 8.11: Synthesis of Compounds (265), (266)

STEP 1: Synthesis of methyl trans-4-[[3-fluoro-4-(hydroxymethyl)phenyl]methyl]cyclohexanecarboxylate—Compound of formula (IIIb) in Scheme 3, as in example 8.2 here-above

STEP 2: Synthesis of trans-4-[[3-fluoro-4-(hydroxymethyl)phenyl]methyl]cyclohexanecarboxylic acid—Compound (Iia) in Scheme 1—in a similar way as performed in Example 1.1, but using methyl trans-4-[[3-fluoro-4-(hydroxymethyl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[3-fluoro-4-(hydroxymethyl)phenyl]methyl]cyclohexanecarbonyl]isoxazolidin-3-yl]benzonitrile (Compound (266)) is performed as in STEP 5c of Example 1.4, but starting from trans-4-[[3-fluoro-4-(hydroxymethyl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[[3-fluoro-4-(hydroxymethyl)phenyl]methyl]cyclohexyl]methanone (Compound (265)) is performed as in STEP 3a here-above but starting from (3S)-3-(3,5-difluorophenyl)isoxazolidin instead of 3-fluoro-5-[(3S)-isoxazolidin-3-yl]benzonitrile hydrochloride.

### Example 8.12: Synthesis of Compounds (245), (246)

STEP 1: Synthesis of methyl trans-4-(pyrazin-2-ylmethyl)cyclohexanecarboxylate—Compound of formula (IIIb) in Scheme 3, is performed in a similar way as in example 8.2 here-above

STEP 2: Synthesis of trans-4-(pyrazin-2-ylmethyl)cyclohexanecarboxylic acid—Compound (Iia) in Scheme 1—in a similar way as performed in Example 1.1, but using methyl trans-4-(pyrazin-2-ylmethyl)cyclohexanecarboxylate instead of trans-4-(benzimidazol-1-ylmethyl)cyclohexanecarboxylate

STEP 3a: Synthesis of trans-3-fluoro-5-[(3S)-2-[4-(pyrazin-2-ylmethyl)cyclohexanecarbonyl]isoxazolidin-3-yl]benzonitrile (Compound (245)) is performed as in STEP 5c of Example 1.4, but starting from trans-4-(pyrazin-2-ylmethyl)cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-pyrazin-2-ylisoxazolidin-2-yl]-[4-(pyrazin-2-ylmethyl)cyclohexyl]methanone (Compound (246)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.13: Synthesis of Compounds (247), (248)

STEP 1: Synthesis of methyl trans-4-(pyrimidin-5-ylmethyl)cyclohexanecarboxylate—Compound of formula (IIIb) in Scheme 3, is performed in a similar way as in example 8.2 here-above

STEP 2: Synthesis of trans-4-(pyrimidin-5-ylmethyl)cyclohexanecarboxylic acid—Compound (Iia) in Scheme 1—in a similar way as performed in Example 1.1, but using methyl trans-4-(pyrimidin-5-ylmethyl)cyclohexanecarboxylate instead of trans-4-(benzimidazol-1-ylmethyl)cyclohexanecarboxylate

STEP 3a: Synthesis of trans-3-fluoro-5-[(3S)-2-[4-(pyrimidin-5-ylmethyl)cyclohexanecarbonyl]isoxazolidin-3-yl]benzonitrile (Compound (247)) is performed as in STEP 5c of Example 1.4, but starting from trans-4-(pyrimidin-5-ylmethyl)cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-pyrazin-2-ylisoxazolidin-2-yl]-[4-(pyrimidin-5-ylmethyl)cyclohexyl]methanone (Compound (248)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.14: Synthesis of Compounds (167), (168)

STEP 1: Synthesis of methyl trans-4-[(3-methylimidazol-4-yl)methyl]cyclohexanecarboxylate—Compound of formula (IIIb) in Scheme 3, is performed in a similar way as in example 8.2 here-above

STEP 2: Synthesis of trans-4-[(3-methylimidazol-4-yl)methyl]cyclohexanecarboxylic acid—Compound (Iia) in Scheme 1—in a similar way as performed in Example 1.1, but using methyl trans-4-[(3-methylimidazol-4-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(3-methylimidazol-4-yl)methyl]cyclohexanecarbonyl]isoxazolidin-3-yl]benzonitrile (Compound (167)) is performed as in STEP 5c of Example 1.4, but starting from trans-4-[(3-methylimidazol-4-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-5-[(3S)-2-[4-[(3-methylimidazol-4-yl)methyl]cyclohexanecarbonyl]isoxazolidin-3-yl]pyridine-3-carbonitrile (Compound (168)) is performed as in STEP 3a here-above but starting from 5-[(3S)-2-isoxazolidin-3-yl]pyridine-3-carbonitrile instead of 3-fluoro-5-[(3S)-isoxazolidin-3-yl]benzonitrile hydrochloride.

### Example 8.15: Synthesis of Compounds (239), (240) and (360)

STEP 1: Synthesis of methyl trans-4-[(2-methylthiazol-4-yl)methyl]cyclohexanecarboxylate—Compound of formula (IIb) in Scheme 3, is performed in a similar way as in example 8.2 here-above

STEP 2: Synthesis of trans-4-[(2-methylthiazol-4-yl)methyl]cyclohexanecarboxylic acid—Compound (Iia) in Scheme 1—in a similar way as performed in Example 1.1, but using methyl trans-4-[(2-methylthiazol-4-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(2-methylthiazol-4-yl)methyl]cyclohexanecarbonyl]isoxazolidin-3-yl]benzonitrile (Compound (239)) is performed as in STEP 5c of Example 1.4, but starting from trans-4-[(2-methylthiazol-4-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(2-methylthiazol-4-yl)methyl]cyclohexyl]-[(3S)-3-pyrazin-2-ylisoxazolidin-2-yl]methanone (Compound (240)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-(S)-3-(5-fluoro-6-methylpyridin-3-yl)-1,2-oxazolidin-2-yl]-[4-[(2-methyl-1,3-thiazol-4-yl)methyl]cyclohexyl]methanone (Compound (360)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride

Chiral SFC: RT 1.03 min (100%, Method X)

### Example 8.16: Synthesis of Compounds (263), (264), (309), (343), (344), (346)

STEP 1: Synthesis of methyl trans-4-[(2-methylpyrimidin-5-yl)methyl]cyclohexanecarboxylate—Compound of formula (IIIb) in Scheme 3, is performed in a similar way as in example 8.2 here-above

STEP 2: Synthesis of trans-4-[(2-methylpyrimidin-5-yl)methyl]cyclohexanecarboxylic acid—Compound (Iia) in Scheme 1—in a similar way as performed in Example 1.1, but using methyl trans-4-[(2-methylpyrimidin-5-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(2-methylpyrimidin-5-yl)methyl]cyclohexanecarbonyl]isoxazolidin-3-yl]benzonitrile (Compound (263)) is performed as in STEP 5c of Example 1.4, but starting from trans-4-[(2-methylpyrimidin-5-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[(2-methylpyrimidin-5-yl)methyl]cyclohexyl]methanone (Compound (264)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3c: Synthesis of trans-[(3S)-3-(3,4-difluorophenyl)isoxazolidin-2-yl]-[4-[(2-methylpyrimidin-5-yl)methyl]cyclohexyl]methanone (Compound (309)) is performed as in STEP 3a here-above but starting from (3S)-3-(3,4-difluorophenyl)isoxazolidine instead of 3-fluoro-5-[(3S)-isoxazolidin-3-yl]benzonitrile hydrochloride.

STEP 3c: Synthesis of trans-[(3S)-3-(5-fluoro-6-methylpyridin-3-yl)-1,2-oxazolidin-2-yl]-[4-[(2-methylpyrimidin-5-yl)methyl]cyclohexyl]methanone (Compound (343)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride.

Chiral SFC: RT 2.056 min (100%, Method AH)

STEP 3c: Synthesis of trans-[(3S)-3-(6-methylpyridin-3-yl)-1,2-oxazolidin-2-yl]-[4-[(2-methylpyrimidin-5-yl)methyl]cyclohexyl]methanone (Compound (344)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride.

Chiral SFC: RT 1.13 min (100%, Method X)

STEP 3c: Synthesis of trans-[4-[(2-methylpyrimidin-5-yl)methyl]cyclohexyl]-[(3S)-3-pyrazin-2-yl-1,2-oxazolidin-2-yl]methanone (Compound (346)) is performed as in STEP 3a here-above but starting from ((3S)-3-pyrazin-2-ylisoxazolidine instead of 3-fluoro-5-[(3S)-isoxazolidin-3-yl]benzonitrile hydrochloride.

Chiral SFC: RT 1.08 min (100%, Method X)

### Example 8.17: Synthesis of Compounds (303), (304), (357), (387) and (388)

STEP 1: Synthesis of methyl trans-4-[(6-methylpyridazin-3-yl)methyl]cyclohexanecarboxylate—Compound of formula (IIIb) in Scheme 3, is performed in a similar way as in example 8.2 here-above

STEP 2: Synthesis of trans-4-[(6-methylpyridazin-3-yl)methyl]cyclohexanecarboxylic acid—Compound (Iia) in Scheme 1—in a similar way as performed in Example 1.1, but using methyl trans-4-[(6-methylpyridazin-3-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(6-methylpyridazin-3-yl)methyl]cyclohexanecarbonyl]isoxazolidin-3-yl]benzonitrile (Compound (303)) is performed as in STEP 5c of Example 1.4, but starting from trans-4-[(6-methylpyridazin-3-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[(6-methylpyridazin-3-yl)methyl]cyclohexyl]methanone (Compound (304)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3b: Synthesis of trans-[4-[(6-methylpyridazin-3-yl)methyl]cyclohexyl]-[(3S)-3-pyrazin-2-ylisoxazolidin-2-yl]methanone (Compound (357)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

Chiral SFC: RT 1.24 min (100%, Method X)

STEP 3b: Synthesis of trans-[4-[(6-methylpyridazin-3-yl)methyl]cyclohexyl]-[(3S)-3-(6-methylpyridin-3-yl)-1,2-oxazolidin-2-yl]methanone (Compound (387)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride.

Chiral SFC: RT 1.24 min (100%, Method X)

STEP 3b: Synthesis of trans-[(3S)-3-(5-fluoro-6-methylpyridin-3-yl)-1,2-oxazolidin-2-yl]-[4-[(6-methylpyridazin-3-yl)methyl]cyclohexyl]methanone (Compound (388)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride.

Chiral SFC: RT 1.20 min (100%, Method X)

### Example 8.18: Synthesis of Compounds (300), (301), (319) and (365)

### STEP 1: Synthesis of 6-bromo-3,8-dimethyl-[1,2,4]triazolo[4,3-a]pyridine

### Step a: Synthesis of (5-bromo-3-methyl-2-pyridyl)hydrazine

A mixture of 5-bromo-2-chloro-3-methyl-pyridine (2.5 g, 12.11 mmol, 1 eq.) and NH₂NH₂·H₂O (7.13 g, 121.08 mmol, 6.92 ml, 85% purity, 10 eq.) in n-BuOH (25 ml) was degassed and purged with N₂ for 3 times, and then the mixture was stirred at 130° C. for 72 hrs under N₂ atmosphere. The reaction mixture was concentrated under reduced pressure and the yellow residue was purified by trituration with water and methyl alcohol to give the title compound (2.41 g, crude) as a white solid.

LC/MS: m/z 202.1 [M+H]⁺, Rt 0.839 min (LC/MS method B)

### Step b: Synthesis of 6-bromo-3,8-dimethyl-[1,2,4]triazolo[4,3-a]pyridine

To a solution of (5-bromo-3-methyl-2-pyridyl)hydrazine (2.41 g, 11.93 mmol, 1 eq.) in dioxane (24 ml) was added Ac₂O (1.46 g, 14.31 mmol, 1.34 ml, 1.2 eq.). The mixture was stirred at 25° C. for 0.5 hrs. Acetic acid (21.49 g, 357.83 mmol, 20.47 ml, 30 eq.) was added to the mixture and stirred at 100° C. for 3 hrs. The reaction mixture was concentrated under reduced pressure to remove the solvent. The residue was diluted with water (50 ml) and NaHCO₃ saturated aqueous solution (20 ml) and extracted with EA (20 ml×3). The combined organic layers were washed with saturated salt solution (30 ml), dried over Na₂SO₄, filtered and concentrated under reduced pressure. The residue was purified by column chromatography (SiO₂, petroleum ether/EA=1/1 to 0/1) to give the title compound (1.47 g, 6.52 mmol, 55% yield) as a white solid.

LC/MS: m/z 228.1 [M+H]*, Rt 0.256 min (LC/MS method B).

¹H NMR (400 MHz, CDCl₃): δ ppm 7.90 (d, J=0.6 Hz, 1H), 7.11-7.08 (m, 1H), 2.74 (s, 3H), 2.67 (s, 3H).

STEP 2: Synthesis of methyl trans-4-[(3,8-dimethyl-[1,2,4]triazolo[4,3-a]pyridin-6-yl)methyl]cyclohexanecarboxylate—Compound of formula (IIIb) in Scheme 3, as in example 9.2 here-above, but using 6-bromo-3,8-dimethyl-[1,2,4]triazolo[4,3-a]pyridine instead of 6-bromo-2-methyl-imidazo[1,2-a]pyridine

STEP 3: Synthesis of trans-4-[(3,8-dimethyl-[1,2,4]triazolo[4,3-a]pyridin-6-yl)methyl]cyclohexanecarboxylic acid—Compound (Iia) in Scheme 1—in a similar way as performed in Example 1.1, but using methyl trans-4-[(3,8-dimethyl-[1,2,4]triazolo[4,3-a]pyridin-6-yl)methyl]cyclohexanecarboxylate instead of trans-4-(benzimidazol-1-ylmethyl)cyclohexanecarboxylate

STEP 4a: Synthesis of trans-3-[(3S)-2-[4-[(3,8-dimethyl-[1,2,4]triazolo[4,3-a]pyridin-6-yl)methyl]cyclohexanecarbonyl]-1,2-oxazolidin-3-yl]-5-fluorobenzonitrile (Compound (300)) is performed as in STEP 5c of Example 1.4, but starting from trans-4-[(3,8-dimethyl-[1,2,4]triazolo[4,3-a]pyridin-6-yl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-[4-[(3,8-dimethyl-[1,2,4]triazolo[4,3-a]pyridin-6-yl)methyl]cyclohexyl]-[(3S)-3-pyrazin-2-yl-1,2-oxazolidin-2-yl]methanone (Compound (301)) is performed as in STEP 3a here-above but starting from (3S)-3-pyrazin-2-yl-1,2-oxazolidine instead of 3-fluoro-5-[(3S)-isoxazolidin-3-yl]benzonitrile hydrochloride.

STEP 4c: Synthesis of trans-[4-[(3,8-dimethyl-[1,2,4]triazolo[4,3-a]pyridin-6-yl)methyl]cyclohexyl]-[(3S)-3-(4-fluorophenyl)isoxazolidin-2-yl]methanone (Compound (319)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 4c: Synthesis of trans-[4-(3,8-Dimethyl-[1,2,4]triazolo[4,3-a]pyridin-6-ylmethyl)-cyclohexyl]-[(S)-3-(6-methyl-pyridin-3-yl)-isoxazolidin-2-yl]-methanone (Compound (365)) is performed as in STEP 3aa here-above but starting from (3S)-3-(6-Methyl-3-pyridyl)isoxazolidine instead of 3-fluoro-5-[(3S)-isoxazolidin-3-yl]benzonitrile hydrochloride.

### Example 8.19: Synthesis of Compounds (211) and (214)

### STEP 1: Synthesis of 5-(3-bromo-5-fluoro-phenyl)-1-methyl-pyrazole

Through a suspension of K₂CO₃ (550 mg, 4.00 mmol), Pd(PPh₃)₄(120 mg, 0.10 mmol), 1-methyl-5-(4,4,5,5-tetramethyl-1,3,2-dioxaborolan-2-yl)pyrazole (420 mg, 2.00 mmol) and 1,3-dibromo-5-fluoro-benzene (510 mg, 2.00 mmol) in dioxane (6 ml) and water (0.1 ml) Ar was bubbled. The vial was sealed and heated to 80° C. for 1.5 hrs. The resulting mixture was cooled to rt and partitioned between EA and water. The aqueous layer extracted with EA and the combined organic layers were dried over Na₂SO₄, filtered and concentrated to afford the crude title compound, which was purified by column chromatography (SiO₂; EA/heptane gradient) (180 mg, 710 μmol, 35% yield).

¹H NMR (400 MHz, DMSO-d₆): δ ppm 7.63 (m, 2H), 7.49 (m, 2H), 6.54 (d, J=1.83 Hz, 1H), 3.89 (s, 3H).

STEP 2: Synthesis of methyl trans-4-[[3-fluoro-5-(2-methylpyrazol-3-yl)phenyl]methyl]cyclohexanecarboxylate—Compound of formula (IIIb) in Scheme 3, as in example 9.2 here-above but using 5-(3-bromo-5-fluoro-phenyl)-1-methyl-pyrazole instead of 6-bromo-2-methyl-imidazo[1,2-a]pyridine

Synthesis of trans-4-[[3-fluoro-5-(2-methylpyrazol-3-yl)phenyl]methyl]cyclohexanecarboxylic acid—Compound (Iia) in Scheme 1—in a similar way as performed in Example 1.1, but using methyl trans-4-[[3-fluoro-5-(2-methylpyrazol-3-yl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[3-fluoro-5-(2-methylpyrazol-3-yl)phenyl]methyl]cyclohexanecarbonyl]isoxazolidin-3-yl]benzonitrile (Compound (214)) is performed as in STEP 5c of Example 1.4, but starting from trans-4-[[3-fluoro-5-(2-methylpyrazol-3-yl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[[3-fluoro-5-(2-methylpyrazol-3-yl)phenyl]methyl]cyclohexyl]-[(3S)-3-pyrazin-2-ylisoxazolidin-2-yl]methanone (Compound (211)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.20: Synthesis of Compound (305)

STEP 1: Synthesis of 3-bromo-5-(2,5-dimethylpyrazol-3-yl)pyridine as performed in Example 8.19 here-above

STEP 2: Synthesis of methyl trans-4-[[5-(2,5-dimethylpyrazol-3-yl)-3-pyridyl]methyl]cyclohexanecarboxylate—Compound of formula (IIIb) in Scheme 3, as in example 9.2 here-above but using 3-bromo-5-(2,5-dimethylpyrazol-3-yl)pyridine instead of 6-bromo-2-methyl-imidazo[1,2-a]pyridine

STEP 3: Synthesis of trans-4-[[5-(2,5-dimethylpyrazol-3-yl)-3-pyridyl]methyl]cyclohexanecarboxylic acid—Compound (Iia) in Scheme 1—in a similar way as performed in Example 1.1, but using methyl trans-4-[[5-(2,5-dimethylpyrazol-3-yl)-3-pyridyl]methyl]cyclohexanecarboxylate instead of trans-4-(benzimidazol-1-ylmethyl)cyclohexanecarboxylate

STEP 4: Synthesis of trans-[4-[[5-(2,5-dimethylpyrazol-3-yl)-3-pyridyl]methyl]cyclohexyl]-[(3S)-3-pyrazin-2-ylisoxazolidin-2-yl]methanone (Compound (305)) is performed as in STEP 5c of Example 1.4, but starting from trans-4-[[5-(2,5-dimethylpyrazol-3-yl)-3-pyridyl]methyl]cyclohexanecarboxylic acid and (3S)-3-pyrazin-2-ylisoxazolidine instead of trans-4-[(5-cyano-6-fluoro-benzimidazol-1-yl)methyl]cyclohexanecarboxylic acid and respectively 3-fluoro-5-[(3S)-isoxazolidin-3-yl]benzonitrile hydrochloride.

### Example 8.21: Synthesis of Compound (308)

STEP 1: Synthesis of 3-bromo-5-(1,3-dimethylpyrazol-4-yl)pyridine as performed in Example 8.19 here-above

STEP 2: Synthesis of methyl trans-4-[[5-(1,3-dimethylpyrazol-4-yl)-3-pyridyl]methyl]cyclohexanecarboxylate—Compound of formula (IIIb) in Scheme 3, as in example 9.2 here-above but using 3-bromo-5-(1,3-dimethylpyrazol-4-yl)pyridine instead of 6-bromo-2-methyl-imidazo[1,2-a]pyridine

STEP 3: Synthesis of trans-4-[[5-(1,3-dimethylpyrazol-4-yl)-3-pyridyl]methyl]cyclohexanecarboxylic acid—Compound (Iia) in Scheme 1—in a similar way as performed in Example 1.1, but using methyl trans-4-[[5-(1,3-dimethylpyrazol-4-yl)-3-pyridyl]methyl]cyclohexanecarboxylate instead of trans-4-(benzimidazol-1-ylmethyl)cyclohexanecarboxylate

STEP 4: Synthesis of trans-[4-[[5-(1,3-dimethylpyrazol-4-yl)-3-pyridyl]methyl]cyclohexyl]-[(3S)-3-pyrazin-2-ylisoxazolidin-2-yl]methanone (Compound (308)) is performed as in STEP 5c of Example 1.4, but starting from trans-4-[[5-(1,3-dimethylpyrazol-4-yl)-3-pyridyl]methyl]cyclohexanecarboxylic acid and (3S)-3-pyrazin-2-ylisoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

### Example 8.22: Synthesis of Compounds (249), (250), (334), (335), (336), (347), (362) and (409)

STEP 1: Synthesis of methyl trans-4-[(8-methyl-[1,2,4]triazolo[1,5-a]pyridin-6-yl)methyl]cyclohexanecarboxylate—Compound of formula (IIIb) in Scheme 3, as in example 8.2 here-above

STEP 2: Synthesis of trans-4-[(8-methyl-[1,2,4]triazolo[1,5-a]pyridin-6-yl)methyl]cyclohexanecarboxylic acid—Compound (Iia) in Scheme 1—in a similar way as performed in Example 1.1, but using methyl trans-4-[(8-methyl-[1,2,4]triazolo[1,5-a]pyridin-6-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(8-methyl-[1,2,4]triazolo[1,5-a]pyridin-6-yl)methyl]cyclohexanecarbonyl]-1,2-oxazolidin-3-yl]benzonitrile (Compound (249)) is performed as in STEP 5c of Example 1.4, but starting from trans-4-[(8-methyl-[1,2,4]triazolo[1,5-a]pyridin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(8-methyl-[1,2,4]triazolo[1,5-a]pyridin-6-yl)methyl]cyclohexyl]-[(3S)-3-pyrazin-2-yl-1,2-oxazolidin-2-yl]methanone (Compound (250)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-[(3S)-3-(5-methylpyridin-3-yl)-1,2-oxazolidin-2-yl]-[4-[(8-methyl-[1,2,4]triazolo[1,5-a]pyridin-6-yl)methyl]cyclohexyl]methanone (Compound (334)) is performed as in STEP 3a here-above but starting from (3S)-3-(5-methyl-3-pyridyl)isoxazolidine instead of 3-fluoro-5-[(3S)-isoxazolidin-3-yl]benzonitrile hydrochloride.

Chiral SFC: RT 1.26 min (100%, Method X)

STEP 3d: Synthesis of trans-[(3S)-3-(5-fluoro-6-methylpyridin-3-yl)-1,2-oxazolidin-2-yl]-[4-[(8-methyl-[1,2,4]triazolo[1,5-a]pyridin-6-yl)methyl]cyclohexyl]methanone (Compound (335)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride.

Chiral SFC: RT 1.21 min (99.2%, Method X)

STEP 3e: Synthesis of trans-[(3S)-3-(5-fluoropyridin-3-yl)-1,2-oxazolidin-2-yl]-[4-[(8-methyl-[1,2,4]triazolo[1,5-a]pyridin-6-yl)methyl]cyclohexyl]methanone (Compound (336)) is performed as in STEP 3a here-above but starting from 3-(5-Fluoro-3-pyridyl)isoxazolidine instead of 3-fluoro-5-[(3S)-isoxazolidin-3-yl]benzonitrile hydrochloride.

Chiral SFC: RT 1.22 min (99.7%, Method X)

STEP 3e: Synthesis of trans-[(3S)-3-(6-methylpyridin-3-yl)-1,2-oxazolidin-2-yl]-[4-[(8-methyl-[1,2,4]triazolo[1,5-a]pyridin-6-yl)methyl]cyclohexyl]methanone (Compound (347)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride.

Chiral SFC: RT 1.37 min (100%, Method X)

STEP 3f: Synthesis of trans-[(S)-3-(2-Methyl-thiazol-4-yl)-isoxazolidin-2-yl]-[4-(8-methyl-[1,2,4]triazolo[1,5-a]pyridin-6-ylmethyl)-cyclohexyl]-methanone (Compound (362)) is performed as in STEP 3a here-above but starting from (3S)-3-(2-Methylthiazol-4-yl)isoxazolidine instead of 3-fluoro-5-[(3S)-isoxazolidin-3-yl]benzonitrile hydrochloride

STEP 3g: Synthesis of trans-[(3S)-3-(5-methylpyrazin-2-yl)-1,2-oxazolidin-2-yl]-[4-[(8-methyl-[1,2,4]triazolo[1,5-a]pyridin-6-yl)methyl]cyclohexyl]methanone (Compound (409)) is performed as in STEP 3a here-above but starting from (3S)-3-(5-methylpyrazin-2-yl)isoxazolidine instead of 3-fluoro-5-[(3S)-isoxazolidin-3-yl]benzonitrile hydrochloride

Chiral SFC: RT 0.66 min (100%, Method X)

### Example 8.23: Synthesis of Compounds (292), (293)

STEP 1: Synthesis of methyl trans-4-[(2,7-dimethylimidazo[1,2-b]pyridazin-6-yl)methyl]cyclohexanecarboxylate—Compound of formula (IIIb) in Scheme 3,is performed in a similar way as in example 8.2 here-above

STEP 2: Synthesis of trans-4-[(2,7-dimethylimidazo[1,2-b]pyridazin-6-yl)methyl]cyclohexanecarboxylic acid—Compound (Iia) in Scheme 1—in a similar way as performed in Example 1.1, but using methyl trans-4-[(2,7-dimethylimidazo[1,2-b]pyridazin-6-yl)methyl]cyclohexanecarboxylate instead of trans-4-(benzimidazol-1-ylmethyl)cyclohexanecarboxylate

STEP 3a: Synthesis of trans-3-[(3S)-2-[4-[(2,7-dimethylimidazo[1,2-b]pyridazin-6-yl)methyl]cyclohexanecarbonyl]isoxazolidin-3-yl]-5-fluoro-benzonitrile (Compound (292)) is performed as in STEP 5c of Example 1.4, but starting from trans-4-[(2,7-dimethylimidazo[1,2-b]pyridazin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[(2,7-dimethylimidazo[1,2-b]pyridazin-6-yl)methyl]cyclohexyl]methanone (Compound (293)) is performed as in STEP 3′ here-above but starting from (3S)-3-(3,5-difluorophenyl)isoxazolidine instead of 3-fluoro-5-[(3S)-isoxazolidin-3-yl]benzonitrile hydrochloride.

### Example 8.24: Synthesis of Compound (281), (350), (351) and (370)

STEP 1: Synthesis of methyl trans-4-[(8-fluoro-2-methyl-imidazo[1,2-a]pyridin-6-yl)methyl]cyclohexanecarboxylate—Compound of formula (IIIb) in Scheme 3, is performed in a similar way as in example 8.2 here-above

STEP 2: Synthesis of trans-4-[(8-fluoro-2-methyl-imidazo[1,2-a]pyridin-6-yl)methyl]cyclohexanecarboxylic acid—Compound (Iia) in Scheme 1—in a similar way as performed in Example 1.1, but using trans-4-[(8-fluoro-2-methyl-imidazo[1,2-a]pyridin-6-yl)methyl]cyclohexanecarboxylate instead of trans-4-(benzimidazol-1-ylmethyl)cyclohexanecarboxylate

STEP 3a: Synthesis of trans-[(3S)-3-(3,5-difluorophenyl)isoxazolidin-2-yl]-[4-[(8-fluoro-2-methyl-imidazo[1,2-a]pyridin-6-yl)methyl]cyclohexyl]methanone (Compound (281)) is performed as in STEP 5c of Example 1.4, but starting from trans-4-[(8-fluoro-2-methyl-imidazo[1,2-a]pyridin-6-yl)methyl]cyclohexanecarboxylic acid and (3S)-3-(3,5-difluorophenyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

STEP 3b: Synthesis of trans-[4-(8-Fluoro-2-methyl-imidazo[1,2-a]pyridin-6-ylmethyl)-cyclohexyl]-((S)-3-pyrazin-2-yl-isoxazolidin-2-yl)-methanone (Compound (350)) is performed as in STEP 3a here-above but starting from (3S)-3-pyrazin-2-ylisoxazolidine instead of (3S)-3-(3,5-difluorophenyl)isoxazolidine.

STEP 3c: Synthesis of trans-[4-[(8-fluoro-2-methylimidazo[1,2-a]pyridin-6-yl)methyl]cyclohexyl]-[(3S)-3-(6-methylpyridin-3-yl)-1,2-oxazolidin-2-yl]methanone (Compound (351)) is performed as in STEP 3a here-above but starting from (3S)-3-(6-Methyl-3-pyridyl)isoxazolidine instead of (3S)-3-(3,5-difluorophenyl)isoxazolidine.

Chiral SFC: RT 1.54 min (96.4%, Method 1)

STEP 3d: Synthesis of trans-[4-(8-Fluoro-2-methyl-imidazo[1,2-a]pyridin-6-ylmethyl)-cyclohexyl]-[(S)-3-(2-methyl-thiazol-4-yl)-isoxazolidin-2-yl]-methanone (Compound (370)) is performed as in STEP 3a here-above but starting from (3S)-3-(2-Methylthiazol-4-yl)isoxazolidine instead of (3S)-3-(3,5-difluorophenyl)isoxazolidine.

### Example 8.25: Synthesis of Compounds (284), (285), (327), (333), (340), (341), (379), (380)

### STEP 1: Synthesis of 6-bromo-8-fluoro-2-methyl-[1,2,4]triazolo[1,5-a]pyridine

### Step a: Synthesis of N′-(5-bromo-3-fluoro-2-pyridyl)-N,N-dimethyl-acetamidine

To a solution of 5-bromo-3-fluoro-pyridin-2-amine (1.7 g, 8.90 mmol, 1 eq.) in DMF (20 ml) was added 1,1-dimethoxy-N,N-dimethyl-ethanamine (2.37 g, 17.80 mmol, 2.60 ml, 2 eq.). The mixture was stirred at 130° C. for 3 hrs. The reaction mixture was concentrated under reduced pressure to give crude N′-(5-bromo-3-fluoro-2-pyridyl)-N,N-dimethyl-acetamidine (2.4 g, crude) as a brown solid was used for the next step directly.

### Step b: Synthesis of N-(5-bromo-3-fluoro-2-pyridyl)-N′-hydroxy-acetamidine

A mixture of N′-(5-bromo-3-fluoro-2-pyridyl)-N,N-dimethyl-acetamidine (2.4 g, 9.23 mmol, 1 eq.), NH₂OhxHCl (833.55 mg, 12.00 mmol, 1.3 eq.) in IPA (40 ml) was degassed and purged with N₂ for 3 times, and then the mixture was stirred at 50° C. for 3 hrs under N₂ atmosphere. The reaction mixture was diluted with water (100 ml) and extracted with EA (100 ml×3). The combined organic layers were washed with brine 100 ml, dried over Na₂SO₄, filtered and concentrated under reduced pressure to give N-(5-bromo-3-fluoro-2-pyridyl)-N′-hydroxy-acetamidine (3 g, crude) as a brown solid was used for the next step directly.

### Step c: Synthesis of 6-bromo-8-fluoro-2-methyl-[1,2,4]triazolo[1,5-a]pyridine

To a solution of N-(5-bromo-3-fluoro-2-pyridyl)-N′-hydroxy-acetamidine (3 g, 12.09 mmol, 1 eq.) in THF (25 ml) was added TFAA (2.79 g, 13.30 mmol, 1.85 ml, 1.1 eq.) at 0° C. The reaction mixture was stirred at 25° C. for 3 hrs. The reaction mixture was adjusted to pH 7 with NaHCO₃, and then diluted with water (50 ml) and extracted with EA (50 ml×3). The combined organic layers were washed with brine (50 ml), dried over Na₂SO₄, filtered and concentrated under reduced pressure to give a residue, which was triturated with petroleum ether/EA=5/1 at 25° C. for 30 min to give 6-bromo-8-fluoro-2-methyl-[1,2,4]triazolo[1,5-a]pyridine (1.35 g, 5.81 mmol, 48% yield) as a white solid.

STEP 2: Synthesis of methyl trans-4-[(8-fluoro-2-methyl-[1,2,4]triazolo[1,5-a]pyridin-6-yl)methyl]cyclohexanecarboxylate—Compound of formula (IIIb) in Scheme 3, as in example 9.2 here-above but using 6-bromo-8-fluoro-2-methyl-[1,2,4]triazolo[1,5-a]pyridine instead of 6-bromo-2-methyl-imidazo[1,2-a]pyridine

STEP 3: Synthesis of trans-4-[(8-fluoro-2-methyl-[1,2,4]triazolo[1,5-a]pyridin-6-yl)methyl]cyclohexanecarboxylic acid—Compound (Iia) in Scheme 1—in a similar way as performed in Example 1.1, but using methyl trans-4-[(8-fluoro-2-methyl-[1,2,4]triazolo[1,5-a]pyridin-6-yl)methyl]cyclohexanecarboxylate instead of trans-4-(benzimidazol-1-ylmethyl)cyclohexanecarboxylate

STEP 4a: Synthesis of trans-3-fluoro-5-[(3S)-2-[4-[(8-fluoro-2-methyl-[1,2,4]triazolo[1,5-a]pyridin-6-yl)methyl]cyclohexanecarbonyl]isoxazolidin-3-yl]benzonitrile (Compound (284)) is performed as in STEP 5c of Example 1.4, but starting from trans-4-[(8-fluoro-2-methyl-[1,2,4]triazolo[1,5-a]pyridin-6-yl)methyl]cyclohexanecarboxylic acid instead of trans-4-[(5-cyano-6-fluoro-benzimidazol-1-yl)methyl]cyclohexanecarboxylic acid.

STEP 4b: Synthesis of trans-[(3S)-3-(3,5-difluorophenyl)isoxazolidin-2-yl]-[4-[(8-fluoro-2-methyl-[1,2,4]triazolo[1,5-a]pyridin-6-yl)methyl]cyclohexyl]methanone (Compound (285)) is performed as in STEP 4a here-above but starting from (3S)-3-(3,5-difluorophenyl)isoxazolidine instead of 3-fluoro-5-[(3S)-isoxazolidin-3-yl]benzonitrile hydrochloride.

STEP 4c: Synthesis of trans-[(3S)-3-(5-fluoro-6-methyl-3-pyridyl)isoxazolidin-2-yl]-[4-[(8-fluoro-2-methyl-[1,2,4]triazolo[1,5-a]pyridin-6-yl)methyl]cyclohexyl]methanone (Compound (327)) is performed as in STEP 4a here-above but starting from ((3S)-3-(5-Fluoro-6-methyl-3-pyridyl)isoxazolidine instead of 3-fluoro-5-[(3S)-isoxazolidin-3-yl]benzonitrile hydrochloride.

Chiral SFC: RT 1.19 min (100%, Method W)

STEP 4d: Synthesis of trans-[4-[(8-fluoro-2-methyl-[1,2,4]triazolo[1,5-a]333yrdine-6-yl)methyl]cyclohexyl]-[(3S)-3-(5-methylpyridin-3-yl)-1,2-oxazolidin-2-yl]methanone (Compound (333)) is performed as in STEP 4a here-above but starting from (3S)-3-(5-methyl-3-pyridyl)isoxazolidine instead of 3-fluoro-5-[(3S)-isoxazolidin-3-yl]benzonitrile hydrochloride.

Chiral SFC: RT 1.31 min (100%, Method X)

STEP 4e: Synthesis of trans-[4-[(8-fluoro-2-methyl-[1,2,4]triazolo[1,5-a]pyridin-6-yl)methyl]cyclohexyl]-[(3S)-3-pyrazin-2-yl-1,2-oxazolidin-2-yl]methanone (Compound (340)) is performed as in STEP 4a here-above but starting from (3S)-3-pyrazin-2-ylisoxazolidine instead of 3-fluoro-5-[(3S)-isoxazolidin-3-yl]benzonitrile hydrochloride.

Chiral SFC: RT 1.78 min (100%, Method AL)

STEP 4f: Synthesis of trans-[4-[(8-fluoro-2-methyl-[1,2,4]triazolo[1,5-a]pyridin-6-yl)methyl]cyclohexyl]-[(3S)-3-(6-methylpyridin-3-yl)-1,2-oxazolidin-2-yl]methanone (Compound (341)) is performed as in STEP 4a here-above but starting from (3S)-3-(6-Methyl-3-pyridyl)isoxazolidine instead of 3-fluoro-5-[(3S)-isoxazolidin-3-yl]benzonitrile hydrochloride.

Chiral SFC: RT 1.46 min (100%, Method AR)

STEP 4g: Synthesis of trans-[4-[(8-fluoro-2-methyl-[1,2,4]triazolo[1,5-a]pyridin-6-yl)methyl]cyclohexyl]-[(3S)-3-(5-methylpyrazin-2-yl)-1,2-oxazolidin-2-yl]methanone (Compound (379)) is performed as in STEP 4a here-above but starting from (3S)-3-(5-methylpyrazin-2-yl)isoxazolidine instead of 3-fluoro-5-[(3S)-isoxazolidin-3-yl]benzonitrile hydrochloride.

Chiral SFC: RT 1.77 min (100%, Method Z)

STEP 4h: Synthesis of trans-4-[(8-fluoro-2-methyl-[1,2,4]triazolo[1,5-a]pyridin-6-yl)methyl]cyclohexyl]-[(3S)-3-(5-fluoropyridin-3-yl)-1,2-oxazolidin-2-yl]methanone (Compound (380)) is performed as in STEP 4a here-above but starting 3-(5-Fluoro-3-pyridyl)isoxazolidine instead of 3-fluoro-5-[(3S)-isoxazolidin-3-yl]benzonitrile hydrochloride.

Chiral SFC: RT 1.73 min (100%, Method X)

### Example 8.26: Synthesis of Compounds (290), (291)

STEP 1: Synthesis of methyl trans-4-[(2,8-dimethylimidazo[1,2-b]pyridazin-6-yl)methyl]cyclohexanecarboxylate—Compound of formula (IIIb) in Scheme 3, is performed as in example 8.2 here-above

STEP 2: Synthesis of trans-4-[(2,8-dimethylimidazo[1,2-b]pyridazin-6-yl)methyl]cyclohexanecarboxylic acid—Compound (Iia) in Scheme 1—in a similar way as performed in Example 1.1, but using methyl trans-4-[(2,8-dimethylimidazo[1,2-b]pyridazin-6-yl)methyl]cyclohexanecarboxylate instead of trans-4-(benzimidazol-1-ylmethyl)cyclohexanecarboxylate

STEP 3a: Synthesis of trans-3-[(3S)-2-[4-[(2,8-dimethylimidazo[1,2-b]pyridazin-6-yl)methyl]cyclohexanecarbonyl]isoxazolidin-3-yl]-5-fluoro-benzonitrile (Compound (290)) is performed as in STEP 5c of Example 1.4, but starting from trans-4-[(2,8-dimethylimidazo[1,2-b]pyridazin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[(2,8-dimethylimidazo[1,2-b]pyridazin-6-yl)methyl]cyclohexyl]methanone (Compound (291)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 8.27: Synthesis of Compounds (169), (170), (171), (183), (348), (352) and (369)

STEP 1: Synthesis of methyl trans-4-[[4-(3-hydroxyoxetan-3-yl)phenyl]methyl]cyclohexanecarboxylate (typical example of cross electrophile coupling as detailed in ACS Catal. 2020, 10, 12642-12656)—Compound of formula (IIIb) in Scheme 3 In a vial under an Ar atmosphere NiBr₂ ethylene glycol dimethyl ether complex (6.2 mg, 20 μmol) and 4,4′-di-tert-butyl-2,2′-bipyridine (5.4 mg, 20 μmol) were suspended in 4 ml dioxane. The suspension was sonicated at 40° C. in an ultrasound bath for 20 min to form the catalyst suspension.

3-(4-bromophenyl)oxetan-3-ol (229 mg, 1.00 mmol), methyl trans-4-(bromomethyl)cyclohexanecarboxylate (282 mg, 1.20 mmol), and cobalt(II)phthalocyanine (CAS 3317-67-7; 29 mg, 50 μmol) were placed in a vial under an Ar atmosphere. The catalyst suspension was added and Ar was bubbled through the resulting suspension for 10 min. Tetrakis(dimethylamino)-ethylene (TDAE) (240 mg, 1.20 mmol) was added and the vial sealed and heated to 80° C. for 24 hrs. The mixture was cooled to rt and the resulting residue partitioned between EA and 0.1 N HCl. The aqueous layer was extracted with EA and the combined organic layers were dried over Na₂SO₄, filtered and concentrated to afford the crude title compound, which was purified by column chromatography (SiO₂; EA/heptane gradient; 0-100% EA) (200 mg, 657 μmol, 66% yield).

¹H NMR (400 MHz, DMSO-d₆): δ ppm 7.48 (d, J=8.25 Hz, 2H), 7.17 (d, J=8.25 Hz, 2H), 6.23 (s, 1H), 4.74 (d, J=6.79 Hz, 2H), 4.67 (d, J=6.79 Hz, 2H), 3.56 (s, 3H), 2.45 (m, 2H), 2.23 (m, 1H), 1.86 (m, 2H), 1.67 (m, 2H), 1.47 (m, 1H), 1.26 (m, 2H), 0.98 (m, 2H).

STEP 2: Synthesis of trans-4-[[4-(3-hydroxyoxetan-3-yl)phenyl]methyl]cyclohexanecarboxylic acid—Compound (Iia) in Scheme 1—in a similar way as performed in Example 1.1, but using methyl trans-4-[[4-(3-hydroxyoxetan-3-yl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[4-(3-hydroxyoxetan-3-yl)phenyl]methyl]cyclohexanecarbonyl]isoxazolidin-3-yl]benzonitrile (Compound (169)) is performed as in STEP 5c of Example 1.4, but starting from trans-4-[[4-(3-hydroxyoxetan-3-yl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(4-fluorophenyl)isoxazolidin-2-yl]-[4-[[4-(3-hydroxyoxetan-3-yl)phenyl]methyl]cyclohexyl]methanone (Compound (170)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3c: Synthesis of trans-[(3S)-3-(3,5-difluorophenyl)isoxazolidin-2-yl]-[4-[[4-(3-hydroxyoxetan-3-yl)phenyl]methyl]cyclohexyl]methanone (Compound (171)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3d: Synthesis of trans-5-[(3S)-2-[4-[[4-(3-hydroxyoxetan-3-yl)phenyl]methyl]cyclohexanecarbonyl]isoxazolidin-3-yl]pyridine-3-carbonitrile (Compound (183)) is performed as in STEP 3a here-above but starting from 5-[(3S)-isoxazolidin-3-yl]pyridine-3-carbonitrile instead of 3-fluoro-5-[(3S)-isoxazolidin-3-yl]benzonitrile hydrochloride.

STEP 3e: Synthesis of trans-5 [4-[[4-(3-hydroxyoxetan-3-yl)phenyl]methyl]cyclohexyl]-[(3S)-3-(6-methylpyridin-3-yl)-1,2-oxazolidin-2-yl]methanone (Compound (348)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride.

Chiral SFC: RT 1.83 min (100%, Method AR)

STEP 3f: Synthesis of trans-[4-[[4-(3-hydroxyoxetan-3-yl)phenyl]methyl]cyclohexyl]-[(3S)-3-pyrazin-2-yl-1,2-oxazolidin-2-yl]methanone (Compound (352)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

Chiral SFC: RT 1.37 min (100%, Method F)

STEP 3g: Synthesis of trans-{4-[4-(3-Hydroxy-oxetan-3-yl)-benzyl]-cyclohexyl}-[(S)-3-(2-methyl-thiazol-4-yl)-isoxazolidin-2-yl]-methanone (Compound (369)) is performed as in STEP 3a here-above but starting from (3S)-3-(2-Methylthiazol-4-yl)isoxazolidine instead of 3-fluoro-5-[(3S)-isoxazolidin-3-yl]benzonitrile hydrochloride.

### Example 8.28: Synthesis of Compounds (296), (297) and (329)

STEP 1: Synthesis of methyl trans-4-[(1-methylpyrazolo[4,3-b]pyridin-6-yl)methyl]cyclohexanecarboxylate—Compound of formula (IIb) in Scheme 3—is performed as in example 8.27 here-above

STEP 2: Synthesis of trans-4-[(1-methylpyrazolo[4,3-b]pyridin-6-yl)methyl]cyclohexanecarboxylic acid—Compound (Iia) in Scheme 1—in a similar way as performed in Example 1.1, but using methyl trans-4-[(1-methylpyrazolo[4,3-b]pyridin-6-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(1-methylpyrazolo[4,3-b]pyridin-6-yl)methyl]cyclohexanecarbonyl]isoxazolidin-3-yl]benzonitrile (Compound (296)) is performed as in STEP 5c of Example 1.4, but starting from trans-4-[(1-methylpyrazolo[4,3-b]pyridin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[(1-methylpyrazolo[4,3-b]pyridin-6-yl)methyl]cyclohexyl]methanone (Compound (297)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3c: Synthesis of trans-[4-[(1-methylpyrazolo[4,3-b]pyridin-6-yl)methyl]cyclohexyl]-[(3S)-3-(6-methylpyridin-3-yl)-1,2-oxazolidin-2-yl]methanone (Compound (329)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride.

Chiral SFC: RT 1.60 min (100%, Method AR)

### Example 8.29: Synthesis of Compounds (1), (221), (222), (223), (260), (261), (262), (289), (321), (355), (372), (373), (374)

STEP 1: Synthesis of methyl trans-4-[(2-methyl-[1,2,4]triazolo[1,5-a]pyridin-6-yl)methyl]cyclohexanecarboxylate—Compound of formula (IIIb) in Scheme 3—is performed as in example 8.27 here-above

STEP 2: Synthesis of trans-4-[(2-methyl-[1,2,4]triazolo[1,5-a]pyridin-6-yl)methyl]cyclohexanecarboxylic acid—Compound (Iia) in Scheme 1—in a similar way as performed in Example 1.1, but using methyl trans-4-[(2-methyl-[1,2,4]triazolo[1,5-a]pyridin-6-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(2-methyl-[1,2,4]triazolo[1,5-a]pyridin-6-yl)methyl]cyclohexanecarbonyl]isoxazolidin-3-yl]benzonitrile (Compound (221)) is performed as in STEP 5c of Example 1.4, but starting from trans-4-[(2-methyl-[1,2,4]triazolo[1,5-a]pyridin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-3-[(3S)-2-[4-[(2-methyl-[1,2,4]triazolo[1,5-a]pyridin-7-yl)methyl]cyclohexanecarbonyl]isoxazolidin-3-yl]benzonitrile (Compound (1)) is performed as in STEP 3a here-above but starting from 5-[(3S)-isoxazolidin-3-yl]benzonitrile hydrochloride instead of 3-fluoro-5-[(3S)-isoxazolidin-3-yl]benzonitrile hydrochloride.

STEP 3c: Synthesis of trans-[4-[(2-methyl-[1,2,4]triazolo[1,5-a]pyridin-6-yl)methyl]cyclohexyl]-[(3S)-3-pyrazin-2-ylisoxazolidin-2-yl]methanone (Compound (222)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3d: Synthesis of trans-[(3S)-3-(3,5-difluorophenyl)isoxazolidin-2-yl]-[4-[(2-methyl-[1,2,4]triazolo[1,5-a]pyridin-6-yl)methyl]cyclohexyl]methanone (Compound (223)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3e: Synthesis of trans-[(3S)-3-(4-fluorophenyl)isoxazolidin-2-yl]-[4-[(2-methyl-[1,2,4]triazolo[1,5-a]pyridin-6-yl)methyl]cyclohexyl]methanone (Compound (260)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3f: Synthesis of trans-[(3S)-3-(3,4-difluorophenyl)isoxazolidin-2-yl]-[4-[(2-methyl-[1,2,4]triazolo[1,5-a]pyridin-6-yl)methyl]cyclohexyl]methanone (Compound (261)) is performed as in STEP 3a here-above but starting from (3S)-3-(3,4-difluorophenyl)isoxazolidine instead of 3-fluoro-5-[(3S)-isoxazolidin-3-yl]benzonitrile hydrochloride.

STEP 3g: Synthesis of trans-5-[(3S)-2-[4-[(2-methyl-[1,2,4]triazolo[1,5-a]pyridin-6-yl)methyl]cyclohexanecarbonyl]isoxazolidin-3-yl]pyridine-3-carbonitrile (Compound (262)) is performed as in STEP 3a here-above but starting from 5-[(3S)-isoxazolidin-3-yl]pyridine-3-carbonitrile instead of 3-fluoro-5-[(3S)-isoxazolidin-3-yl]benzonitrile hydrochloride.

STEP 3h: Synthesis of trans-4-fluoro-3-[(3S)-2-[4-[(2-methyl-[1,2,4]triazolo[1,5-a]pyridin-6-yl)methyl]cyclohexanecarbonyl]isoxazolidin-3-yl]benzonitrile (Compound (289)) is performed as in STEP 3a here-above but starting from 4-fluoro-3-[(3S)-isoxazolidin-3-yl]benzonitrile instead of 3-fluoro-5-[(3S)-isoxazolidin-3-yl]benzonitrile hydrochloride.

STEP 3i: Synthesis of trans-[(3S)-3-(4-fluorophenyl)isoxazolidin-2-yl]-[4-[(2-methyl-[1,2,4]triazolo[1,5-a]pyridin-7-yl)methyl]cyclohexyl]methanone (Compound (321)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3j: Synthesis of trans-[4-[(2-methyl-[1,2,4]triazolo[1,5-a]pyridin-7-yl)methyl]cyclohexyl]-[(3S)-3-(5-fluoro-6-methyl-3-pyridyl)isoxazolidin-2-yl]-methanone (Compound (355) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

Chiral SFC: RT 1.17 min (100%, Method X)

STEP 3k: Synthesis of trans-[(S)-3-(2-Methyl-thiazol-4-yl)-isoxazolidin-2-yl]-[4-(2-methyl-[1,2,4]triazolo[1,5-a]pyridin-7-ylmethyl)-cyclohexyl]-methanone (Compound (372)) is performed as in STEP 3a here-above but starting from (3S)-3-(2-Methylthiazol-4-yl)isoxazolidine instead of 3-fluoro-5-[(3S)-isoxazolidin-3-yl]benzonitrile hydrochloride.

STEP 3l: Synthesis of trans-[(S)-3-(6-Methyl-pyridin-3-yl)-isoxazolidin-2-yl]-[4-(2-methyl-[1,2,4]triazolo[1,5-a]pyridin-7-ylmethyl)-cyclohexyl]-methanone (Compound (373)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride.

STEP 3m: Synthesis of trans-[4-(2-Methyl-[1,2,4]triazolo[1,5-a]pyridin-7-ylmethyl)-cyclohexyl]-((S)-3-pyrazin-2-yl-isoxazolidin-2-yl)-methanone (Compound (374)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

Chiral SFC: RT 1.26 min (100%, Method X)

### Example 8.30: Synthesis of Compounds (172), (173), (174), (175), (176), (177)

STEP 1: Synthesis of methyl trans-4-[[2-fluoro-5-(methylcarbamoyl)phenyl]methyl]cyclohexanecarboxylate—Compound of formula (IIIb) in Scheme 3—is performed in a similar way as in example 8.27 here-above

STEP 2: Synthesis of trans-4-[[2-fluoro-5-(methylcarbamoyl)phenyl]methyl]cyclohexanecarboxylic acid—Compound (Iia) in Scheme 1—in a similar way as performed in Example 1.1, but using methyl trans-4-[[2-fluoro-5-(methylcarbamoyl)phenyl]methyl]cyclohexanecarboxylate instead of trans-4-(benzimidazol-1-ylmethyl)cyclohexanecarboxylate

STEP 3a: Synthesis of trans-3-[[4-[(3S)-3-(3-cyano-5-fluoro-phenyl)isoxazolidine-2-carbonyl]cyclohexyl]methyl]-4-fluoro-N-methyl-benzamide (Compound (177)) is performed as in STEP 5c of Example 1.4, but starting from trans-4-[[2-fluoro-5-(methylcarbamoyl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-4-fluoro-3-[[4-[(3S)-3-(5-fluoro-3-pyridyl)isoxazolidine-2-carbonyl]cyclohexyl]methyl]-N-methyl-benzamide (Compound (172)) is performed as in STEP 3a here-above but starting from (3S)-3-(5-fluoro-3-pyridyl)isoxazolidine instead of 3-fluoro-5-[(3S)-isoxazolidin-3-yl]benzonitrile hydrochloride.

STEP 3c: Synthesis of trans-4-fluoro-3-[[4-[(3S)-3-(4-fluorophenyl)isoxazolidine-2-carbonyl]cyclohexyl]methyl]-N-methyl-benzamide (Compound (173)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3d: Synthesis of trans-3-[[4-[(3S)-3-(3,5-difluorophenyl)isoxazolidine-2-carbonyl]cyclohexyl]methyl]-4-fluoro-N-methyl-benzamide (Compound (174)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3e: Synthesis of trans-4-fluoro-N-methyl-3-[[4-[(3S)-3-pyrazin-2-ylisoxazolidine-2-carbonyl]cyclohexyl]methyl]benzamide (Compound (175)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3f: Synthesis of trans-3-[[4-[(3S)-3-(5-cyano-3-pyridyl)isoxazolidine-2-carbonyl]cyclohexyl]methyl]-4-fluoro-N-methyl-benzamide (Compound (176)) is performed as in STEP 3a here-above but starting from (3S)-3-(5-cyano-3-pyridyl)isoxazolidine instead of 3-fluoro-5-[(3S)-isoxazolidin-3-yl]benzonitrile hydrochloride.

### Example 8.31: Synthesis of Compounds (160), (161), (162), (163), (164), (165)

STEP 1: Synthesis of methyl trans-4-[[3-fluoro-5-(methylcarbamoyl)phenyl]methyl]cyclohexanecarboxylate—Compound of formula (IIIb) in Scheme 3—is performed in a similar way as in example 8.27 here-above

STEP 2: Synthesis of trans-4-[[3-fluoro-5-(methylcarbamoyl)phenyl]methyl]cyclohexanecarboxylic acid—Compound (Iia) in Scheme 1—in a similar way as performed in Example 1.1, but using methyl trans-4-[[3-fluoro-5-(methylcarbamoyl)phenyl]methyl]cyclohexanecarboxylate instead of trans-4-(benzimidazol-1-ylmethyl)cyclohexanecarboxylate

STEP 3a: Synthesis of trans-3-[[4-[(3S)-3-(3-cyano-5-fluoro-phenyl)isoxazolidine-2-carbonyl]cyclohexyl]methyl]-5-fluoro-N-methyl-benzamide (Compound (164)) is performed as in STEP 5c of Example 1.4, but starting from trans-4-[[3-fluoro-5-(methylcarbamoyl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-3-fluoro-5-[[4-[(3S)-3-(5-fluoro-3-pyridyl)isoxazolidine-2-carbonyl]cyclohexyl]methyl]-N-methyl-benzamide (Compound (160)) is performed as in STEP 3a here-above but starting from (3S)-3-(5-fluoro-3-pyridyl)isoxazolidine instead of 3-fluoro-5-[(3S)-isoxazolidin-3-yl]benzonitrile hydrochloride.

STEP 3c: Synthesis of trans-3-[[4-[(3S)-3-(3,5-difluorophenyl)isoxazolidine-2-carbonyl]cyclohexyl]methyl]-5-fluoro-N-methyl-benzamide (Compound (161)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3d: Synthesis of trans-3-fluoro-N-methyl-5-[[4-[(3S)-3-pyrazin-2-ylisoxazolidine-2-carbonyl]cyclohexyl]methyl]benzamide (Compound (162)) is performed as in STEP 2a here-above but starting from (3S)-3-pyrazin-2-ylisoxazolidine instead of 3-fluoro-5-[(3S)-isoxazolidin-3-yl]benzonitrile hydrochloride.

STEP 3e: Synthesis of trans-3-[[4-[(3S)-3-(5-cyano-3-pyridyl)isoxazolidine-2-carbonyl]cyclohexyl]methyl]-5-fluoro-N-methyl-benzamide (Compound (163)) is performed as in STEP 3a here-above but starting from (3S)-3-(5-cyano-3-pyridyl)isoxazolidine instead of 3-fluoro-5-[(3S)-isoxazolidin-3-yl]benzonitrile hydrochloride.

STEP 3f: Synthesis of trans-3-fluoro-5-[[4-[(3S)-3-(4-fluorophenyl)isoxazolidine-2-carbonyl]cyclohexyl]methyl]-N-methyl-benzamide (Compound (165)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

### Example 8.32: Synthesis of Compounds (144), (145), (154), (155), (156), (157)

STEP 1: Synthesis of methyl trans-4-[(3-oxoisoindolin-5-yl)methyl]cyclohexanecarboxylate—Compound of formula (IIIb) in Scheme 3—is performed in a similar way as in example 8.27 here-above

STEP 2: Synthesis of trans-4-[(3-oxoisoindolin-5-yl)methyl]cyclohexanecarboxylic acid—Compound (Iia) in Scheme 1—in a similar way as performed in Example 1.1, but using methyl trans-4-[(3-oxoisoindolin-5-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(3-oxoisoindolin-5-yl)methyl]cyclohexanecarbonyl]isoxazolidin-3-yl]benzonitrile (Compound (144)) is performed as in STEP 5c of Example 1.4, but starting from trans-4-[(3-oxoisoindolin-5-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-6-[[4-[(3S)-3-pyrazin-2-ylisoxazolidine-2-carbonyl]cyclohexyl]methyl]isoindolin-1-one (Compound (145)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-6-[[4-[(3S)-3-(4-fluorophenyl)isoxazolidine-2-carbonyl]cyclohexyl]methyl]isoindolin-1-one (Compound (154)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3d: Synthesis of trans-6-[[4-[(3S)-3-(5-fluoro-3-pyridyl)isoxazolidine-2-carbonyl]cyclohexyl]methyl]isoindolin-1-one (Compound (155)) is performed as in STEP 3a here-above but starting from (3S)-3-(5-fluoro-3-pyridyl)isoxazolidine instead of 3-fluoro-5-[(3S)-isoxazolidin-3-yl]benzonitrile hydrochloride.

STEP 3e: Synthesis of trans-6-[[4-[(3S)-3-(3,5-difluorophenyl)isoxazolidine-2-carbonyl]cyclohexyl]methyl]isoindolin-1-one (Compound (156)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3f: Synthesis of trans-6-[[4-[(3S)-3-(6-methyl-3-pyridyl)isoxazolidine-2-carbonyl]cyclohexyl]methyl]isoindolin-1-one (Compound (157)) is performed as in STEP 3a here-above but starting from (3S)-3-(6-methyl-3-pyridyl)isoxazolidine instead of 3-fluoro-5-[(3S)-isoxazolidin-3-yl]benzonitrile hydrochloride.

### Example 8.33: Synthesis of Compounds (294), (295)

STEP 1: Synthesis of methyl trans-4-[(1-methylindazol-6-yl)methyl]cyclohexanecarboxylate—Compound of formula (IIIb) in Scheme 3—is performed in a similar way as in example 8.27 here-above

STEP 2: Synthesis of trans-4-[(1-methylindazol-6-yl)methyl]cyclohexanecarboxylic acid—Compound (Iia) in Scheme 1—in a similar way as performed in Example 1.1, but using methyl trans-4-[(1-methylindazol-6-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(1-methylindazol-6-yl)methyl]cyclohexanecarbonyl]isoxazolidin-3-yl]benzonitrile (Compound (294)) is performed as in STEP 5c of Example 1.4, but starting from trans-4-[(1-methylindazol-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[(3S)-3-(3,5-difluorophenyl)isoxazolidin-2-yl]-[4-[(1-methylindazol-6-yl)methyl]cyclohexyl]methanone (Compound (295)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 8.34: Synthesis of Compounds (207), (208), (209), (210), (216), (220)

STEP 1: Synthesis of 1-(3-bromo-5-fluoro-phenyl)-2-methyl-imidazole

A suspension of K₂CO₃ (550 mg, 4.00 mmol), 2-methylimidazole (246 mg, 3 mmol), and 1-bromo-3,5-difluoro-benzene (386 mg, 2 mmol) in DMF (4 ml) was heated to 130° C. for 3 hrs. The resulting mixture was cooled to rt and partitioned between EA and water. The aqueous layer was extracted with EA and the combined organic layers were dried over Na₂SO₄, filtered and concentrated to afford the crude title compound, which was purified by column chromatography (SiO₂; EA/heptane gradient; EA 50-100%) to give the title compound (150 mg, 590 μmol, 30% yield).

¹H NMR (400 MHz, DMSO-d₆): δ ppm 7.67 (m, 1H), 7.61 (m, 1H), 7.51 (m, 1H), 7.37 (d, J=1.83 Hz, 1H), 6.92 (d, J=1.83 Hz, 1H), 3.31 (s, 3H).

STEP 2: Synthesis of methyl trans-4-[[3-fluoro-5-(2-methylimidazol-1-yl)phenyl]methyl]cyclohexanecarboxylate—Compound of formula (IIIb) in Scheme 3—is performed in a similar way as in example 9.28 here-above but using 1-(3-bromo-5-fluoro-phenyl)-2-methyl-imidazole instead of 3-(4-bromophenyl)oxetan-3-ol

STEP 3: Synthesis of trans-4-[[3-fluoro-5-(2-methylimidazol-1-yl)phenyl]methyl]cyclohexanecarboxylic acid—Compound (Iia) in Scheme 1—in a similar way as performed in Example 1.1, but using methyl trans-4-[[3-fluoro-5-(2-methylimidazol-1-yl)phenyl]methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[[3-fluoro-5-(2-methylimidazol-1-yl)phenyl]methyl]cyclohexanecarbonyl]isoxazolidin-3-yl]benzonitrile (Compound (216)) is performed as in STEP 5c of Example 1.4, but starting from trans-4-[[3-fluoro-5-(2-methylimidazol-1-yl)phenyl]methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[[3-fluoro-5-(2-methylimidazol-1-yl)phenyl]methyl]cyclohexyl]-[(3S)-3-(4-fluorophenyl)isoxazolidin-2-yl]methanone (Compound (207)) is performed as in STEP 3a here-above but starting from (3S)-3-(4-fluorophenyl)isoxazolidine instead of 3-fluoro-5-[(3S)-isoxazolidin-3-yl]benzonitrile hydrochloride. STEP 3c: Synthesis of trans-[(3S)-3-(3,5-difluorophenyl)isoxazolidin-2-yl]-[4-[[3-fluoro-5-(2-methylimidazol-1-yl)phenyl]methyl]cyclohexyl]methanone (Compound (208)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3d: Synthesis of trans-[4-[[3-fluoro-5-(2-methylimidazol-1-yl)phenyl]methyl]cyclohexyl]-[(3S)-3-pyrazin-2-ylisoxazolidin-2-yl]methanone (Compound (209)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3e: Synthesis of trans-5-[(3S)-2-[4-[[3-fluoro-5-(2-methylimidazol-1-yl)phenyl]methyl]cyclohexanecarbonyl]isoxazolidin-3-yl]pyridine-3-carbonitrile (Compound (210)) is performed as in STEP 3a here-above but starting from 5-[(3S)-isoxazolidin-3-yl]pyridine-3-carbonitrile instead of 3-fluoro-5-[(3S)-isoxazolidin-3-yl]benzonitrile hydrochloride.

STEP 3f: Synthesis of trans-[4-[[3-fluoro-5-(2-methylimidazol-1-yl)phenyl]methyl]cyclohexyl]-[(3S)-3-(5-fluoro-3-pyridyl)isoxazolidin-2-yl]methanone (Compound (220)) is performed as in STEP 3a here-above but starting from (3S)-3-(5-fluoro-3-pyridyl)isoxazolidine instead of 3-fluoro-5-[(3S)-isoxazolidin-3-yl]benzonitrile hydrochloride.

### Example 8.35: Synthesis of Compounds (242), (243), (244)

STEP 1: Synthesis of methyl trans-4-[(3-cyano-4-fluoro-phenyl)methyl]cyclohexanecarboxylate—Compound of formula (IIIb) in Scheme 3—is performed in a similar way as in example 8.27 here-above

STEP 2: Synthesis of trans-4-[(3-cyano-4-fluoro-phenyl)methyl]cyclohexanecarboxylic acid—Compound (Iia) in Scheme 1—in a similar way as performed in Example 1.1, but using methyl trans-4-[(3-cyano-4-fluoro-phenyl)methyl]cyclohexanecarboxylate instead of trans-4-(benzimidazol-1-ylmethyl)cyclohexanecarboxylate

STEP 3a: Synthesis of trans-5-[[4-[(3S)-3-(3-cyano-5-fluoro-phenyl)isoxazolidine-2-carbonyl]cyclohexyl]methyl]-2-fluoro-benzonitrile (Compound (244)) is performed as in STEP 5c of Example 1.4, but starting from trans-4-[(3-cyano-4-fluoro-phenyl)methyl]cyclohexanecarboxylic acid instead of trans-4-[(5-cyano-6-fluoro-benzimidazol-1-yl)methyl]cyclohexanecarboxylic acid. STEP 3b: Synthesis of trans-2-fluoro-5-[[4-[(3S)-3-(4-fluorophenyl)isoxazolidine-2-carbonyl]cyclohexyl]methyl]benzonitrile (Compound (242)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3c: Synthesis of trans-2-fluoro-5-[[4-[(3S)-3-pyrazin-2-ylisoxazolidine-2-carbonyl]cyclohexyl]methyl]benzonitrile (Compound (243)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.36: Synthesis of Compounds (178), (179), (180), (181), (182), (197), (316)

STEP 1: Synthesis of methyl trans-4-[(5-fluoro-2-methyl-3-pyridyl)methyl]cyclohexanecarboxylate—Compound of formula (IIIb) in Scheme 3—as in example 8.27 here-above

STEP 2: Synthesis of trans-4-[(5-fluoro-2-methyl-pyridyl)methyl]cyclohexanecarboxylic acid—Compound (Iia) in Scheme 1—in a similar way as performed in Example 1.1, but using methyl trans-4-[(5-fluoro-2-methyl-3-pyridyl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(5-fluoro-2-methyl-3-pyridyl)methyl]cyclohexanecarbonyl]isoxazolidin-3-yl]benzonitrile (Compound (181)) is performed as in STEP 5c of Example 1.4, but starting from trans-4-[(5-fluoro-2-methyl-pyridyl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(5-fluoro-2-methyl-3-pyridyl)methyl]cyclohexyl]-[(3S)-3-(5-fluoro-3-pyridyl)isoxazolidin-2-yl]methanone (Compound (178)) is performed as in STEP 3a here-above but starting from (3S)-3-(5-fluoro-3-pyridyl)isoxazolidine instead of 3-fluoro-5-[(3S)-isoxazolidin-3-yl]benzonitrile hydrochloride.

STEP 3c: Synthesis of trans-[4-[(5-fluoro-2-methyl-3-pyridyl)methyl]cyclohexyl]-[(3S)-3-(4-fluorophenyl)isoxazolidin-2-yl]methanone (Compound (179)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3d: Synthesis of trans-[4-[(5-fluoro-2-methyl-3-pyridyl)methyl]cyclohexyl]-[(3S)-3-pyrazin-2-ylisoxazolidin-2-yl]methanone (Compound (180)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3e: Synthesis of trans-[(3S)-3-(3,5-difluorophenyl)isoxazolidin-2-yl]-[4-[(5-fluoro-2-methyl-3-pyridyl)methyl]cyclohexyl]methanone (Compound (182)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3f: Synthesis of (Compound (197)) is performed as in STEP 3a here-above but starting from 5-[(3S)-isoxazolidin-3-yl]pyridine-3-carbonitrile instead of 3-fluoro-5-[(3S)-isoxazolidin-3-yl]benzonitrile hydrochloride.

STEP 3g: Synthesis of (Compound (316)) is performed as in STEP 3a here-above but starting from (3S)-3-(5-methylpyrazin-2-yl)isoxazolidine instead of 3-fluoro-5-[(3S)-isoxazolidin-3-yl]benzonitrile hydrochloride.

### Example 8.37: Synthesis of Compounds (237), (238), (306), (307), (311), (395), (397), (404)

STEP 1: Synthesis of methyl trans-4-[(2-methylpyrazol-3-yl)methyl]cyclohexanecarboxylate—Compound of formula (IIIb) in Scheme 3—is performed in a similar way as in example 8.27 here-above

STEP 2: Synthesis of trans-4-[(2-methylpyrazol-3-yl)methyl]cyclohexanecarboxylic acid—Compound (Iia) in Scheme 1—in a similar way as performed in Example 1.1, but using methyl trans-4-[(2-methylpyrazol-3-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(2-methylpyrazol-3-yl)methyl]cyclohexanecarbonyl]isoxazolidin-3-yl]benzonitrile (Compound (237)) is performed as in STEP 5c of Example 1.4, but starting from trans-4-[(2-methylpyrazol-3-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(2-methylpyrazol-3-yl)methyl]cyclohexyl]-[(3S)-3-pyrazin-2-ylisoxazolidin-2-yl]methanone (Compound (238)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-[(3S)-3-(4-fluorophenyl)isoxazolidin-2-yl]-[4-[(2-methylpyrazol-3-yl)methyl]cyclohexyl]methanone (Compound (306)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3d: Synthesis of trans-[(3S)-3-(3,5-difluorophenyl)isoxazolidin-2-yl]-[4-[(2-methylpyrazol-3-yl)methyl]cyclohexyl]methanone (Compound (307)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3e: Synthesis of trans-[(3S)-3-(3,4-difluorophenyl)isoxazolidin-2-yl]-[4-[(2-methylpyrazol-3-yl)methyl]cyclohexyl]methanone (Compound (311)) is performed as in STEP 3a here-above but starting from (3S)-3-(3,4-difluorophenyl)isoxazolidine instead of 3-fluoro-5-[(3S)-isoxazolidin-3-yl]benzonitrile hydrochloride.

STEP 3f: Synthesis of trans-[(3S)-3-(5-methylpyrazin-2-yl)-1,2-oxazolidin-2-yl]-[4-[(2-methylpyrazol-3-yl)methyl]cyclohexyl]methanone Compound (395)) is performed as in STEP 3a here-above but starting from (3S)-3-(5-methylpyrazin-2-yl)isoxazolidine instead of 3-fluoro-5-[(3S)-isoxazolidin-3-yl]benzonitrile hydrochloride

Chiral SFC: RT 1.032 min (100%, Method X)

STEP 3g: Synthesis of trans-[4-[(2-methylpyrazol-3-yl)methyl]cyclohexyl]-[(3S)-3-(6-methylpyridin-3-yl)-1,2-oxazolidin-2-yl]methanone (Compound (397)) is performed as in STEP 3a here-above but starting from (3S)-3-(6-Methyl-3-pyridyl)isoxazolidine instead of 3-fluoro-5-[(3S)-isoxazolidin-3-yl]benzonitrile hydrochloride

Chiral SFC: RT 1.12 min (100%, Method X)

STEP 3h: Synthesis of trans-[(3S)-3-(5-fluoro-6-methylpyridin-3-yl)-1,2-oxazolidin-2-yl]-[4-[(2-methylpyrazol-3-yl)methyl]cyclohexyl]methanone (Compound (404)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride

Chiral SFC: RT 1.09 min (100%, Method X)

### Example 8.38: Synthesis of Compounds (212), (213), (219)

STEP 1: Synthesis of methyl trans-4-[(7-fluoro-3-oxo-isoindolin-5-yl)methyl]cyclohexanecarboxylate—Compound of formula (IIb) in Scheme 3—is performed in a similar way as in example 8.27 here-above

STEP 2: Synthesis of trans-4-[(7-fluoro-3-oxo-isoindolin-5-yl)methyl]cyclohexanecarboxylic acid—Compound (Iia) in Scheme 1—in a similar way as performed in Example 1.1, but using methyl trans-4-[(7-fluoro-3-oxo-isoindolin-5-yl)methyl]cyclohexanecarboxylate instead of trans-4-(benzimidazol-1-ylmethyl)cyclohexanecarboxylate

STEP 3a: Synthesis trans-3-fluoro-5-[(3S)-2-[4-[(7-fluoro-3-oxo-isoindolin-5-yl)methyl]cyclohexanecarbonyl]isoxazolidin-3-yl]benzonitrile (Compound (219)) is performed as in STEP 5c of Example 1.4, but starting from trans-4-[(7-fluoro-3-oxo-isoindolin-5-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-4-fluoro-6-[[4-[(3S)-3-(4-fluorophenyl)isoxazolidine-2-carbonyl]cyclohexyl]methyl]isoindolin-1-one (Compound (212)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3c: Synthesis of trans-4-fluoro-6-[[4-[(3S)-3-pyrazin-2-ylisoxazolidine-2-carbonyl]cyclohexyl]methyl]isoindolin-1-one (Compound (213)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

### Example 8.39: Synthesis of Compounds (205), (206), (390) and (392)

STEP 1: Synthesis of methyl trans-4-[(8-fluoro-[1,2,4]triazolo[1,5-a]pyridin-6-yl)methyl]cyclohexanecarboxylate—Compound of formula (IIb) in Scheme 3—is performed in as similar way as in example 8.27 here-above

STEP 2: Synthesis of trans-4-[(8-fluoro-[1,2,4]triazolo[1,5-a]pyridin-6-yl)methyl]cyclohexanecarboxylic acid—Compound (Iia) in Scheme 1—in a similar way as performed in Example 1.1, but using methyl trans-4-[(8-fluoro-[1,2,4]triazolo[1,5-a]pyridin-6-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(8-fluoro-[1,2,4]triazolo[1,5-a]pyridin-6-yl)methyl]cyclohexanecarbonyl]isoxazolidin-3-yl]benzonitrile (Compound (206)) is performed as in STEP 5c of Example 1.4, but starting from trans-4-[(8-fluoro-[1,2,4]triazolo[1,5-a]pyridin-6-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(8-fluoro-[1,2,4]triazolo[1,5-a]pyridin-6-yl)methyl]cyclohexyl]-[(3S)-3-pyrazin-2-ylisoxazolidin-2-yl]methanone (Compound (205)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-[4-[(8-fluoro-[1,2,4]triazolo[1,5-a]pyridin-6-yl)methyl]cyclohexyl]-[(3S)-3-(5-methylpyridin-3-yl)-1,2-oxazolidin-2-yl]methanone (Compound (390)) is performed as in STEP 3a here-above but starting from (3S)-3-(5-methyl-3-pyridyl)isoxazolidine instead of 3-fluoro-5-[(3S)-isoxazolidin-3-yl]benzonitrile hydrochloride.

Chiral SFC: RT 1.99 min (100%, Method Z)

STEP 3d: Synthesis of trans-[(3S)-3-(5-fluoro-6-methylpyridin-3-yl)-1,2-oxazolidin-2-yl]-[4-[(8-fluoro-[1,2,4]triazolo[1,5-a]pyridin-6-yl)methyl]cyclohexyl]methanone (Compound (392)) is performed as in STEP 3a here-above but starting from (3S)-3-(5-Fluoro-6-methyl-3-pyridyl)isoxazolidine instead of 3-fluoro-5-[(3S)-isoxazolidin-3-yl]benzonitrile hydrochloride.

Chiral SFC: RT 1.44 min (100%, Method AE)

### Example 8.40: Synthesis of Compounds (150), (151), (235), (236)

Synthesis of methyl trans-4-[(1-methylpyrazol-4-yl)methyl]cyclohexanecarboxylate is performed in a similar way as in Example 8.2 step 1, but using (1-methylpyrazol-4-yl)bromomethyl.

Synthesis of trans-4-[(1-methylpyrazol-4-yl)methyl]cyclohexanecarboxylic acid—Compound (Iia) in Scheme 1—in a similar way as performed in Example 1.1, but using trans-4-[(1-methylpyrazol-4-yl)methyl]cyclohexanecarboxylate instead of trans-4-(benzimidazol-1-ylmethyl)cyclohexanecarboxylate

STEP 3a: Synthesis of trans-3-fluoro-5-[(3S)-2-[4-[(1-methylpyrazol-4-yl)methyl]cyclohexanecarbonyl]isoxazolidin-3-yl]benzonitrile (Compound (150)) is performed as in STEP 5c of Example 1.4, but starting from trans-4-[(1-methylpyrazol-4-yl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-[4-[(1-methylpyrazol-4-yl)methyl]cyclohexyl]-[(3S)-3-pyrazin-2-ylisoxazolidin-2-yl]methanone (Compound (151)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3c: Synthesis of trans-5-[(3S)-2-[4-[(1-methylpyrazol-4-yl)methyl]cyclohexanecarbonyl]isoxazolidin-3-yl]pyridine-3-carbonitrile (Compound (235)) is performed as in STEP 3a here-above but starting from 5-[(3S)-isoxazolidin-3-yl]pyridine-3-carbonitrile instead of 3-fluoro-5-[(3S)-isoxazolidin-3-yl]benzonitrile hydrochloride.

STEP 3d: Synthesis of trans-[(3S)-3-(3,5-difluorophenyl)isoxazolidin-2-yl]-[4-[(1-methylpyrazol-4-yl)methyl]cyclohexyl]methanone (Compound (236)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

### Example 8.41: Synthesis of Compounds (224)

Synthesis of methyl trans-4-[(3-methyl-[1,2,4]triazolo[4,3-a]pyridin-6-yl)methyl]cyclohexanecarboxylate is performed in a similar way as in Example 8.2 step 1, but using (3-methyl-[1,2,4]triazolo[4,3-a]pyridin-6-yl)bromomethyl.

Synthesis of trans-4-[(3-methyl-[1,2,4]triazolo[4,3-a]pyridin-6-yl)methyl]cyclohexanecarboxylic acid—Compound (Iia) in Scheme 1—is performed in a similar way as performed in Example 1.1, but using trans-4-[(3-methyl-[1,2,4]triazolo[4,3-a]pyridin-6-yl)methyl]cyclohexanecarboxylate instead of trans-4-(benzimidazol-1-ylmethyl)cyclohexanecarboxylate

STEP 3: Synthesis of trans-3-fluoro-5-[(3S)-2-[4-[(3-methyl-[1,2,4]triazolo[4,3-a]pyridin-6-yl)methyl]cyclohexanecarbonyl]isoxazolidin-3-yl]benzonitrile (Compound (224)) is performed as in STEP 5c of Example 1.4, but starting from trans-4-[(3-methyl-[1,2,4]triazolo[4,3-a]pyridin-6-yl)methyl]cyclohexanecarboxylic acid instead of trans-4-[(5-cyano-6-fluoro-benzimidazol-1-yl)methyl]cyclohexanecarboxylic acid.

### Example 8.42: Synthesis of Compounds (317) and (318)

STEP 1: Synthesis of methyl trans-4-[(3,7-dimethyl-[1,2,4]triazolo[4,3-a]pyridin-6-yl)methyl]cyclohexanecarboxylate—Compound of formula (IIIb) in Scheme 3, is performed in a similar way as in example 8.2 here-above.

STEP 2: as in example 8.2 here-above.

STEP 3a: Synthesis of trans-[4-[(3,7-dimethyl-[1,2,4]triazolo[4,3-a]pyridin-6-yl)methyl]cyclohexyl]-[(3S)-3-(3-fluorophenyl)isoxazolidin-2-yl]methanone (Compound (317)) is performed as in STEP 5c of Example 1.4, but starting from 4-[(3,7-dimethyl-[1,2,4]triazolo[4,3-a]pyridin-6-yl)methyl]cyclohexanecarboxylic acid and (3S)-3-(3-fluorophenyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

STEP 3b: Synthesis of [(3S)-3-(3,4-difluorophenyl)isoxazolidin-2-yl]-[4-[(3,7-dimethyl-[1,2,4]triazolo[4,3-a]pyridin-6-yl)methyl]cyclohexyl]methanone (Compound (318)) is performed as in STEP 3a here-above but starting from (3S)-3-(3,4-difluorophenyl)isoxazolidine instead of (3S)-3-(3-fluorophenyl)isoxazolidine.

### Example 8.43: Synthesis of Compound (320), (359), (363) and (364)

STEP 1: Synthesis of methyl trans-4-[(3-methyl-[1,2,4]triazolo[4,3-a]pyridin-6-yl)methyl]cyclohexanecarboxylate—Compound of formula (IIIb) in Scheme 3, as in example 8.2 here-above.

STEP 2: as in example 8.2 here-above.

STEP 3a: Synthesis of trans-[4-[(8-fluoro-3-methyl-[1,2,4]triazolo[4,3-a]pyridin-6-yl)methyl]cyclohexyl]-[(3S)-3-(4-fluorophenyl)isoxazolidin-2-yl]methanone (Compound (320)) is performed as in STEP 5c of Example 1.4, but starting from 4-[(8-fluoro-3-methyl-[1,2,4]triazolo[4,3-a]pyridin-6-yl)methyl]cyclohexanecarboxylic acid and (3S)-3-(4-fluorophenyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

STEP 3b: Synthesis of trans-[4-[(8-fluoro-3-methyl-[1,2,4]triazolo[4,3-a]pyridin-6-yl)methyl]cyclohexyl]-[(3S)-3-pyrazin-2-yl-1,2-oxazolidin-2-yl]methanone (Compound (359)) is performed as in STEP 3a here-above but starting from (3S)-3-pyrazin-2-ylisoxazolidine instead of (3S)-3-(4-fluorophenyl)isoxazolidine) Chiral SFC: RT 1.50 min (100%, Method F)

STEP 3c: Synthesis of trans-[4-(8-Fluoro-3-methyl-[1,2,4]triazolo[4,3-a]pyridin-6-ylmethyl)-cyclohexyl]-[(S)-3-(2-methyl-thiazol-4-yl)-isoxazolidin-2-yl]-methanone (Compound (363)) is performed as in STEP 3a here-above but starting from (3S)-3-(2-Methylthiazol-4-yl)isoxazolidine instead of (3S)-3-(4-fluorophenyl)isoxazolidine)

STEP 3b: Synthesis of trans-[4-(8-Fluoro-3-methyl-[1,2,4]triazolo[4,3-a]pyridin-6-ylmethyl)-cyclohexyl]-[(S)-3-(6-methyl-pyridin-3-yl)-isoxazolidin-2-yl]-methanone (Compound (364) is performed as in STEP 3a here-above but starting from (3S)-3-(6-Methyl-3-pyridyl)isoxazolidine instead of (3S)-3-(4-fluorophenyl)isoxazolidine)

### Example 8.44: Synthesis of Compound (321)

STEP 1: Synthesis of methyl 4-[(2-methyl-[1,2,4]triazolo[1,5-a]pyridin-7-yl)methyl]cyclohexanecarboxylate—Compound of formula (IIIb) in Scheme 3, as in example 8.2 here-above.

STEP 2: as in example 8.2 here-above.

STEP 3: Synthesis of trans-[(3S)-3-(4-fluorophenyl)isoxazolidin-2-yl]-[4-[(2-methyl-[1,2,4]triazolo[1,5-a]pyridin-7-yl)methyl]cyclohexyl]methanone (Compound (321)) is performed as in STEP 5c of Example 1.4, but starting from 4-[(2-methyl-[1,2,4]triazolo[1,5-a]pyridin-7-yl)methyl]cyclohexanecarboxylic acid and (3S)-3-(4-fluorophenyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

### Example 8.45: Synthesis of Compounds (322), (323), (324), (349), (353) and (371)

STEP 1: Synthesis of methyl 4-[(2-methylimidazo[1,2-b]pyridazin-7-yl)methyl]cyclohexanecarboxylate—Compound of formula (IIIb) in Scheme 3, as in example 8.2 here-above.

STEP 2: as in example 8.2 here-above.

STEP 3a: Synthesis of [2-cyano-4-[(3S)-2-[4-[(2-methylimidazo[1,2-b]pyridazin-7-yl)methyl]cyclohexanecarbonyl]isoxazolidin-3-yl]phenyl](Compound (322)) is performed as in STEP 5c of Example 1.4, but starting from 4-[(2-methylimidazo[1,2-b]pyridazin-7-yl)methyl]cyclohexanecarboxylic acid and 3-[(3S)-isoxazolidin-3-yl]benzonitrile instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

STEP 3b: Synthesis of [2-fluoro-4-[(3S)-2-[4-[(2-methylimidazo[1,2-b]pyridazin-7-yl)methyl]cyclohexanecarbonyl]isoxazolidin-3-yl]phenyl](Compound (323)) is performed as in STEP 3a here-above but starting from (3S)-3-(3-fluorophenyl)isoxazolidine instead of 3-[(3S)-isoxazolidin-3-yl]benzonitrile.

STEP 3c: Synthesis of trans-[4-[(3S)-2-[4-[(2-methylimidazo[1,2-b]pyridazin-7-yl)methyl]cyclohexanecarbonyl]isoxazolidin-3-yl]phenyl](Compound (324)) is performed as in STEP 3a here-above but starting from (3S)-3-(4-fluorophenyl)isoxazolidine instead of 3-[(3S)-isoxazolidin-3-yl]benzonitrile.

STEP 3c: Synthesis of trams-[4-[(2-methylimidazo[1,2-b]pyridazin-7-yl)methyl]cyclohexyl]-[(3S)-3-pyrazin-2-yl-1,2-oxazolidin-2-yl]methanone (Compound (349)) is performed as in STEP 3a here-above but starting from (3S)-3-pyrazin-2-ylisoxazolidine instead of 3-[(3S)-isoxazolidin-3-yl]benzonitrile.

Chiral SFC: RT 1.27 min (100%, Method F)

STEP 3d: Synthesis of trans-[4-[(2-methylimidazo[1,2-b]pyridazin-7-yl)methyl]cyclohexyl]-[(3S)-3-(6-methylpyridin-3-yl)-1,2-oxazolidin-2-yl]methanone (Compound (353)) is performed as in STEP 3a here-above but starting from (3S)-3-(6-Methyl-3-pyridyl)isoxazolidine instead of 3-[(3S)-isoxazolidin-3-yl]benzonitrile.

Chiral SFC: RT 1.26 min (100%, Method AS)

STEP 3e: Synthesis of trans-[4-(2-Methyl-imidazo[1,2-b]pyridazin-7-ylmethyl)-cyclohexyl]-[(S)-3-(2-methyl-thiazol-4-yl)-isoxazolidin-2-yl]-methanone (Compound (371)) is performed as in STEP 3a here-above but starting from (3S)-3-(2-Methylthiazol-4-yl)isoxazolidine instead of 3-[(3S)-isoxazolidin-3-yl]benzonitrile.

### Example 8.46: Synthesis of Compound (288, 325, 326, 328, 330, 331, 360, 361, 378 and 411)

STEP 1: Synthesis of methyl trans-4-[(2,8-dimethyl-[1,2,4]triazolo[1,5-a]pyridin-6-yl)methyl]cyclohexanecarboxylate—Compound of formula (IIIb) in Scheme 3, as in example 8.2 here-above.

STEP 2: as in example 8.2 here-above.

STEP 3a: Synthesis of trans-3-[(3S)-2-[4-[(2,8-dimethyl-[1,2,4]triazolo[1,5-a]pyridin-6-yl)methyl]cyclohexanecarbonyl]isoxazolidin-3-yl]-5-fluoro-benzonitrile (Compound (288)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and 3-fluoro-5-[(3S)-isoxazolidin-3-yl]benzonitrile hydrochloride.

STEP 3b: Synthesis of trans-[4-[(2,8-dimethyl-[1,2,4]triazolo[1,5-a]pyridin-6-yl)methyl]cyclohexyl]-[(3S)-3-(6-methyl-3-pyridyl)isoxazolidin-2-yl]methanone (Compound (325)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-(6-Methyl-3-pyridyl)isoxazolidine.

Chiral SFC: RT 1.48 min (100%, Method AC)

STEP 3c: Synthesis of trans-[4-[(2,8-dimethyl-[1,2,4]triazolo[1,5-a]pyridin-6-yl)methyl]cyclohexyl]-[(3S)-3-pyrazin-2-ylisoxazolidin-2-yl]methanone (Compound (326)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-(5-methylpyrazin-2-yl)isoxazolidine

Chiral SFC: RT 1.22 min (100%, Method Z)

STEP 3d: Synthesis of trans-[4-[(2,8-dimethyl-[1,2,4]triazolo[1,5-a]pyridin-6-yl)methyl]cyclohexyl]-[(3S)-3-(5-methylpyrazin-2-yl)isoxazolidin-2-yl]methanone; 2,2,2-trifluoroacetic (Compound (328)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-pyrazin-2-ylisoxazolidine hydrochloride salt

Chiral SFC: RT 1.59 min (100%, Method AC)

STEP 3e: Synthesis of trans-[4-[(2,8-dimethyl-[1,2,4]triazolo[1,5-a]pyridin-6-yl)methyl]cyclohexyl]-[(3S)-3-(5-fluoro-6-methylpyridin-3-yl)-1,2-oxazolidin-2-yl]methanone (Compound (330)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-(5-Fluoro-6-methyl-3-pyridyl)isoxazolidine

Chiral SFC: RT 1.20 min (100%, Method AE)

STEP 3f: Synthesis of trans-[4-[(2,8-dimethyl-[1,2,4]triazolo[1,5-a]pyridin-6-yl)methyl]cyclohexyl]-[(3S)-3-(5-fluoropyridin-3-yl)-1,2-oxazolidin-2-yl]methanone (Compound (331)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and 3-(5-Fluoro-3-pyridyl)isoxazolidine

Chiral SFC: RT 1.21 min (100%, Method AE)

STEP 3g: Synthesis of trans-[4-(2,8-Dimethyl-[1,2,4]triazolo[1,5-a]352yridine-6-ylmethyl)-cyclohexyl]-[(S)-3-(2-methyl-thiazol-4-yl)-isoxazolidin-2-yl]-methanone (Compound (361)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-(2-Methylthiazol-4-yl)isoxazolidine

STEP 3h: Synthesis of trans-[4-[(2,8-dimethyl-[1,2,4]triazolo[1,5-a]pyridin-6-yl)methyl]cyclohexyl]-[(3S)-3-(5-methylpyridin-3-yl)-1,2-oxazolidin-2-yl]methanone (Compound (378)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-(5-methyl-3-pyridyl)isoxazolidine

Chiral SFC: RT 1.67 min (100%, Method AH)

STEP 3h: Synthesis of trans-[4-[(2,8-dimethyl-[1,2,4]triazolo[1,5-a]pyridin-6-yl)methyl]cyclohexyl]-[(3S)-3-(6-methylpyrazin-2-yl)isoxazolidin-2-yl]methanone (Compound (411)) is performed as in STEP 5c of Example 1.4, but starting from methyl trans-4-[(2,8-dimethyl-[1,2,4]triazolo[1,5-a]pyridin-6-yl)methyl]cyclohexanecarboxylic acid and (3S)-3-(6-methylpyrazin-2-yl)isoxazolidine

### Example 9: Synthesis of Compounds (70), (71), (72), (73), (74), (75), (76), (77), (78), (79), (80), (81), (102), (103) by Hydrolysis of Hetero Aryl Nitriles

### Example 9.1: Synthesis of Compounds (70), (71)

STEP 1: Synthesis of trans-4-[(6-carbamoyl-5-fluoro-benzimidazol-1-yl)methyl]cyclohexane carboxylic acid from trans-4-[(6-cyano-5-fluoro-benzimidazol-1-yl)methyl]cyclohexane carboxylic acid as synthesized in Example 1.4

To a solution of trans-4-[(6-cyano-5-fluoro-benzimidazol-1-yl)methyl]cyclohexane carboxylic acid (100 mg, 332 μmol) in THF (4 ml) hydrogen peroxide (50%, 40 μl, 663 μmol) was added at rt followed by a solution of lithium hydroxide (19 mg, 797 μmol) in water (4 ml). The resulting solution was stirred at rt for 30 min after which another equivalent of hydrogen peroxide was added. Volatile components were removed under reduced pressure and the remaining aqueous solution diluted with approx. 3 ml water and acidified with 1 N HCl (796 μl, 796 μmol). The obtained suspension was stirred for 10 min and filtered. The title compound was obtained as a white solid, which was used in the next reaction without further purification (76 mg, 72%).

¹H NMR (600 MHz, DMSO-d₆): δ ppm 12.00 (br s, 1H), 8.36 (s, 1H), 7.95 (d, J=6.36 Hz, 1H), 7.62 (br s, 2H), 7.51 (d, J=11.61 Hz, 1H), 4.14 (d, J=7.34 Hz, 2H), 2.12 (m, 1H), 1.84 (m, 3H), 1.54 (br d, J=10.64 Hz, 2H), 1.24 (m, 2H), 1.06 (m, 2H).

STEP 2a: Synthesis of trans-1-((4-((S)-3-(3-cyano-5-fluorophenyl)isoxazolidine-2-carbonyl)cyclohexyl)methyl)-5-fluoro-1H-benzo[d]imidazole-6-carboxamide (Compound (70)) is performed as in STEP 5c of Example 1.4, but starting from trans-4-[(5-carbamoyl-6-fluoro-benzimidazol-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-5-fluoro-1H-benzo[d]imidazole-6-carboxamide (Compound (71)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

### Example 9.2: Synthesis of Compounds (74), (75)

STEP 1: Synthesis of trans-4-[(5-carbamoyl-6-fluoro-benzimidazol-1-yl)methyl]cyclohexanecarboxylic acid from trans-4-[(5-cyano-6-fluoro-benzimidazol-1-yl)methyl]cyclohexanecarboxylic acid as synthesized in Example 1.3 performed as detailed in Example 9.1

STEP 2a: Synthesis of trans-1-((4-((S)-3-(3-cyano-5-fluorophenyl)isoxazolidine-2-carbonyl)cyclohexyl)methyl)-6-fluoro-1H-benzo[d]imidazole-5-carboxamide (Compound (74)) is performed as in STEP 5c of Example 1.4, but starting from trans-4-[(5-carbamoyl-6-fluoro-benzimidazol-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-6-fluoro-1H-benzo[d]imidazole-5-carboxamide (Compound (75)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

### Example 9.3: Synthesis of Compounds (80), (81)

STEP 1: Synthesis of trans-4-[(6-carbamoyl-4-fluoro-benzimidazol-1-yl)methyl]cyclohexanecarboxylic acid from trans-4-[(6-cyano-4-fluoro-benzimidazol-1-yl)methyl]cyclohexanecarboxylic acid as synthesized in Example 1.5 performed as detailed in Example 9.1

STEP 2a: Synthesis of trans-1-((4-((S)-3-(3-cyano-5-fluorophenyl)isoxazolidine-2-carbonyl)cyclohexyl)methyl)-4-fluoro-1H-benzo[d]imidazole-6-carboxamide (Compound (80)) is performed as in STEP 5c of Example 1.4, but starting from trans-4-[(6-carbamoyl-4-fluoro-benzimidazol-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-4-fluoro-1H-benzo[d]imidazole-6-carboxamide (Compound (81)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

### Example 9.4: Synthesis of Compounds (102), (103)

STEP 1: Synthesis of trans-4-[(6-carbamoyl-4-fluoro-3-methyl-indazol-1-yl)methyl]cyclohexanecarboxylic acid from trans-4-[(6-cyano-4-fluoro-3-methyl-indazol-1-yl)methyl]cyclohexanecarboxylic acid as synthesized in Example 2.5 performed as detailed in Example 9.1

STEP 2a: Synthesis of trans-1-((4-((S)-3-(3-cyano-5-fluorophenyl)isoxazolidine-2-carbonyl)cyclohexyl)methyl)-4-fluoro-3-methyl-1H-indazole-6-carboxamide (Compound (102)) is performed as in STEP 5c of Example 1.4, but starting from trans-4-[(6-carbamoyl-4-fluoro-3-methyl-indazol-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-4-fluoro-3-methyl-1H-indazole-6-carboxamide (Compound (103)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

### Example 9.5: Synthesis of Compounds (72), (73)

STEP 1: Synthesis of trans-4-[(5-carbamoylindazol-1-yl)methyl]cyclohexanecarboxylic acid from trans-4-[(5-cyanoindazol-1-yl)methyl]cyclohexanecarboxylic acid as synthesized in Example 3.5 performed as detailed in Example 9.1

STEP 2a: Synthesis of trans-1-((4-((S)-3-(3-cyano-5-fluorophenyl)isoxazolidine-2-carbonyl)cyclohexyl)methyl)-1H-indazole-5-carboxamide (Compound (72)) is performed as in STEP 5c of Example 1.4, but starting from trans-4-[(5-carbamoylindazol-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-1H-indazole-5-carboxamide (Compound (73)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

### Example 9.6: Synthesis of Compounds (78), (79)

STEP 1: Synthesis of trans-4-[(6-carbamoylpyrazolo[4,3-b]pyridin-1-yl)methyl]cyclohexanecarboxylic acid from trans-4-[(6-cyanopyrazolo[4,3-b]pyridin-1-yl)methyl]cyclohexane carboxylic acid as synthesized in Step 2 of Example 3.20 performed as detailed in Example 9.1

STEP 2a: Synthesis of trans-1-((4-((S)-3-(3-cyano-5-fluorophenyl)isoxazolidine-2-carbonyl)cyclohexyl)methyl)-1H-pyrazolo[4,3-b]pyridine-6-carboxamide (Compound (78)) is performed as in STEP 5c of Example 1.4, but starting from trans-4-[(6-carbamoylpyrazolo[4,3-b]pyridin-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-1H-pyrazolo[4,3-b]pyridine-6-carboxamide (Compound (79)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

### Example 9.7: Synthesis of Compounds (76), (77)

STEP 1: Synthesis of trans-4-[(6-carbamoyl-5-fluoro-indol-1-yl)methyl]cyclohexanecarboxylic acid from trans-4-[(6-cyano-5-fluoro-indol-1-yl)methyl]cyclohexanecarboxylic acid as synthesized in Example 3.25 performed as detailed in Example 9.1

STEP 2a: Synthesis of trans-1-((4-((S)-3-(3-cyano-5-fluorophenyl)isoxazolidine-2-carbonyl)cyclohexyl)methyl)-5-fluoro-1H-indole-6-carboxamide (Compound (76)) is performed as in STEP 5c of Example 1.4, but starting from trans-4-[(6-carbamoyl-5-fluoro-indol-1-yl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-1-((4-((S)-3-(3,5-difluorophenyl)isoxazolidine-2-carbonyl)cyclohexyl)methyl)-5-fluoro-1H-indole-6-carboxamide (Compound (77)) is performed as in STEP 2a here-above but starting from (3S)-3-(3,5-difluorophenyl)isoxazolidin instead of 3-fluoro-5-[(3S)-isoxazolidin-3-yl]benzonitrile hydrochloride.

### Example 9.8: Synthesis of Compounds (251) to (255)

STEP 1: Synthesis of methyl trans-4-[(5-carbamoylindol-1-yl)methyl]cyclohexanecarboxylate is performed in a similar way as in example 3.16

STEP 2: Synthesis of methyl trans-4-[(5-carbamoylindol-1-yl)methyl]cyclohexanecarboxylic acid as in example 3.16

STEP 3a: Synthesis of trans-1-[[4-[(3S)-3-pyrazin-2-ylisoxazolidine-2-carbonyl]cyclohexyl]methyl]indole-5-carboxamide (Compound (251)) is performed as in STEP 5a of Example 1.3, but starting from trans-4-[(5-carbamoylindol-1-yl)methyl]cyclohexanecarboxylic acid and (3S)-3-pyrazin-2-ylisoxazolidine instead of trans-4-[(6-cyano-5-fluoro-benzimidazol-1-yl)methyl]cyclohexanecarboxylic acid and 5-[(3S)-isoxazolidin-3-yl]pyridine-3-carbonitrile respectively.

STEP 3b: Synthesis of trans-1-[[4-[(3S)-3-(5-cyano-3-pyridyl)isoxazolidine-2-carbonyl]cyclohexyl]methyl]indole-5-carboxamide (Compound (252)) is performed as in STEP 5a of Example 1.3, but starting from trans-4-[(5-carbamoylindol-1-yl)methyl]cyclohexanecarboxylic acid instead of trans-4-[(6-cyano-5-fluoro-benzimidazol-1-yl)methyl]cyclohexanecarboxylic acid.

STEP 3c: Synthesis of trans-1-[[4-[(3S)-3-(6-methoxypyrazin-2-yl)isoxazolidine-2-carbonyl]cyclohexyl]methyl]indole-5-carboxamide (Compound (253)) is performed as in STEP 3b here-above, but starting from (3S)-3-(6-methoxypyrazin-2-yl)isoxazolidine instead of 5-[(3S)-isoxazolidin-3-yl]pyridine-3-carbonitrile.

STEP 3d: Synthesis of trans-1-[[4-[(3S)-3-(2-methylthiazol-4-yl)isoxazolidine-2-carbonyl]cyclohexyl]methyl]indole-5-carboxamide (Compound (254)) is performed as in STEP 3b here-above, but starting from (3S)-3-(2-methylthiazol-4-yl)isoxazolidine instead of 5-[(3S)-isoxazolidin-3-yl]pyridine-3-carbonitrile.

STEP 3e: Synthesis of trans-1-[[4-[(3S)-3-(2-pyridyl)isoxazolidine-2-carbonyl]cyclohexyl]methyl]indole-5-carboxamide (Compound (255)) is performed as in STEP 3b here-above, but starting from (3S)-3-(2-pyridyl)isoxazolidine instead of 5-[(3S)-isoxazolidin-3-yl]pyridine-3-carbonitrile.

### Example 10: Synthesis of Compounds (131), (132), (133), (138), (158), (159), (166), (191), (192), (193), (194), (195) by Hydrolysis of Aryl Nitriles

### Example 10.1: Synthesis of Compounds (131), (132), (133), (138), (158), (159), (166)

STEP 1: Synthesis of trans-4-[(3-carbamoyl-4-methyl-phenyl)methyl]cyclohexanecarboxylic acid from trans-4-[(3-cyano-4-methyl-phenyl)methyl]cyclohexanecarboxylic acid as synthesized in Example 8.1

To a suspension of trans-4-[(3-cyano-4-methyl-phenyl)methyl]cyclohexanecarboxylic acid (246 mg, 960 μmol) in DMSO (3 ml), EtOH (3 ml) and water (1 ml) hydrogen peroxide (50% in water, 880 μl, 14.4 mmol) and LiOH (136 mg, 5.75 mmol) were added at rt. The suspension was stirred at 60° C. for 1 hr and cooled to rt. Volatile components were removed under reduced pressure (70 mbar) and the suspension was neutralized with 1 N HCl (5.75 ml, 5.75 mmol). The obtained suspension was stirred for 10 min and filtered. The filter cake was rinsed with 0.1 N HCl and the title compound was obtained as a white solid, which was used in the next reaction without further purification (250 mg, 95%).

¹H NMR (400 MHz, DMSO-d₆): δ ppm 11.95 (br s, 1H), 7.63 (br s, 1H), 7.28 (br s, 1H), 7.10 (m, 3H), 2.42 (d, J=6.97 Hz, 2H), 2.31 (s, 3H), 2.10 (m, 1H), 1.86 (m, 2H), 1.67 (m, 2H), 1.44 (m, 1H), 1.23 (m, 2H), 0.96 (m, 2H).

STEP 2a: Synthesis of trans-5-[[4-[(3S)-3-(3-cyano-5-fluoro-phenyl)isoxazolidine-2-carbonyl]cyclohexyl]methyl]-2-methyl-benzamide (Compound (133)) is performed as in STEP 5c of Example 1.4, but starting from trans-4-[(3-carbamoyl-4-fluoro-phenyl)methyl]cyclohexanecarboxylic acid instead of trans-4-[(5-cyano-6-fluoro-benzimidazol-1-yl)methyl]cyclohexanecarboxylic acid.

STEP 2b: Synthesis of trans-2-methyl-5-[[4-[(3S)-3-(p-tolyl)isoxazolidine-2-carbonyl]cyclohexyl]methyl]benzamide (Compound (131)) is performed as in STEP 2a here-above but starting from (3S)-3-(p-tolyl)isoxazolidine instead of 3-fluoro-5-[(3S)-isoxazolidin-3-yl]benzonitrile hydrochloride.

STEP 2c: Synthesis of trans-5-[[4-[(3S)-3-(3,5-difluorophenyl)isoxazolidine-2-carbonyl]cyclohexyl]methyl]-2-methyl-benzamide (Compound (132)) is performed as in STEP 2a here-above but starting from (3S)-3-(3,5-difluorophenyl)isoxazolidine instead of 3-fluoro-5-[(3S)-isoxazolidin-3-yl]benzonitrile hydrochloride.

STEP 2d: Synthesis of trans-2-methyl-5-[[4-[(3S)-3-pyrazin-2-ylisoxazolidine-2-carbonyl]cyclohexyl]methyl]benzamide (Compound (138)) is performed as in STEP 2a here-above but starting from (3S)-3-pyrazin-2-ylisoxazolidine instead of 3-fluoro-5-[(3S)-isoxazolidin-3-yl]benzonitrile hydrochloride.

STEP 2e: Synthesis of trans-5-[[4-[(3S)-3-(5-cyano-3-pyridyl)isoxazolidine-2-carbonyl]cyclohexyl]methyl]-2-methyl-benzamide (Compound (158)) is performed as in STEP 2a here-above but starting from (3S)-3-(5-cyano-3-pyridyl)isoxazolidine instead of 3-fluoro-5-[(3S)-isoxazolidin-3-yl]benzonitrile hydrochloride.

STEP 2f: Synthesis of trans-2-methyl-5-[[4-[(3S)-3-(2-methylthiazol-4-yl)isoxazolidine-2-carbonyl]cyclohexyl]methyl]benzamide (Compound (159)) is performed as in STEP 2a here-above but starting from (3S)-3-(2-methylthiazol-4-yl)isoxazolidine instead of 3-fluoro-5-[(3S)-isoxazolidin-3-yl]benzonitrile hydrochloride.

STEP 2g: Synthesis of trans-5-[[4-[(3S)-3-(6-methoxypyrazin-2-yl)isoxazolidine-2-carbonyl]cyclohexyl]methyl]-2-methyl-benzamide (Compound (166)) is performed as in STEP 2a here-above but starting from (3S)-3-(6-methoxypyrazin-2-yl)isoxazolidine instead of 3-fluoro-5-[(3S)-isoxazolidin-3-yl]benzonitrile hydrochloride.

### Example 10.2: Synthesis of Compounds (191), (192), (193), (194), (195)

STEP 1: Synthesis of trans-4-[(3-carbamoyl-4-fluoro-phenyl)methyl]cyclohexanecarboxylic acid from trans-4-[(3-cyano-4-fluoro-phenyl)methyl]cyclohexanecarboxylic acid as synthesized in Example 9.36, performed as detailed in Example 10.1

STEP 3a: Synthesis of trans-5-[[4-[(3S)-3-(3-cyano-5-fluoro-phenyl)isoxazolidine-2-carbonyl]cyclohexyl]methyl]-2-fluoro-benzamide (Compound (195)) is performed as in STEP 5c of Example 1.4, but starting from trans-4-[(3-carbamoyl-4-fluoro-phenyl)methyl]cyclohexanecarboxylic acid instead of trans-4-[(5-cyano-6-fluoro-benzimidazol-1-yl)methyl]cyclohexanecarboxylic acid.

STEP 3b: Synthesis of trans-2-fluoro-5-[[4-[(3S)-3-(4-fluorophenyl)isoxazolidine-2-carbonyl]cyclohexyl]methyl]benzamide (Compound (191)) is performed as in STEP 3a here-above but starting from (3S)-3-(4-fluorophenyl)isoxazolidine instead of 3-fluoro-5-[(3S)-isoxazolidin-3-yl]benzonitrile hydrochloride.

STEP 3c: Synthesis of trans-5-[[4-[(3S)-3-(3,5-difluorophenyl)isoxazolidine-2-carbonyl]cyclohexyl]methyl]-2-fluoro-benzamide (Compound (192)) is performed as in STEP 3a here-above but starting from (3S)-3-(3,5-difluorophenyl)isoxazolidine instead of 3-fluoro-5-[(3S)-isoxazolidin-3-yl]benzonitrile hydrochloride.

STEP 3d: Synthesis of trans-2-fluoro-5-[[4-[(3S)-3-pyrazin-2-ylisoxazolidine-2-carbonyl]cyclohexyl]methyl]benzamide (Compound (193)) is performed as in STEP 3a here-above but starting from (3S)-3-pyrazin-2-ylisoxazolidine instead of 3-fluoro-5-[(3S)-isoxazolidin-3-yl]benzonitrile hydrochloride.

STEP 3e: Synthesis of trans-5-[[4-[(3S)-3-(5-cyano-3-pyridyl)isoxazolidine-2-carbonyl]cyclohexyl]methyl]-2-fluoro-benzamide (Compound (194)) is performed as in STEP 3a here-above but starting from (3S)-3-(5-cyano-3-pyridyl)isoxazolidine—instead of 3-fluoro-5-[(3S)-isoxazolidin-3-yl]benzonitrile hydrochloride.

### Example 11: Synthesis of Compound (298)

STEP 1: Synthesis of 6-fluoro-1H-indazole-5-carbonitrile—heteroaromatic compound containing a Nitrogen atom as performed in Example 2.1.

STEP 2: Synthesis of the corresponding carboxylate methyl trans-4-[(5-cyano-6-fluoro-indazol-1-yl)methyl]cyclohexanecarboxylate in a similar way as performed in example 3.1 step 1.

STEP 3: Synthesis of 6-fluoro-1-[[trans-4-[(3S)-3-(6-methyl-3-pyridyl)isoxazolidine-2-carbonyl]cyclohexyl]methyl]indazole-5-carbonitrile (Compound (298)) is performed as in STEP 5c of Example 1.4, but starting from trans-4-[(5-cyano-6-fluoro-indazol-1-yl)methyl]cyclohexanecarboxylic acid and (3S)-3-(6-methyl-3-pyridyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

### Example 12: Synthesis of Compound (299)

STEP 1 and STEP 2: Synthesis of from trans-4-[(3-carbamoyl-4-fluoro-phenyl)methyl]cyclohexanecarboxylic acid as synthesized in Example 10.2.

STEP 3: Synthesis of trans-2-fluoro-5-[[4-[(3S)-3-(6-methylpyrazin-2-yl)isoxazolidine-2-carbonyl]cyclohexyl]methyl]benzamide (Compound (299)) is performed as in STEP 5c of Example 1.4, but starting from trans-4-[(3-carbamoyl-4-fluoro-phenyl)methyl]cyclohexanecarboxylic acid and (3S)-3-(6-methylpyrazin-2-yl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile hydrochloride respectively.

### Example 13: Synthesis of Compound (4)

### Example 13.1: Synthesis of [(3S)-3-(3,5-difluorophenyl)isoxazolidin-2-yl]-[trans-4-(hydroxymethyl)cyclohexyl]methanone

To a stirred solution of trans-4-(hydroxymethyl)cyclohexane-1-carboxylic acid (1.1 g, 6.95 mmol), NEtiPr₂ (3.64 ml, 20.9 mmol) and (S)-3-(3,5-difluorophenyl)isoxazolidine hydrochloride (2.00 g, 9.04 mmol) in DMF (26 ml) HATU (3.17 g, 8.34 mmol) was added at rt. Stirring at rt was continued for 2 hrs. The reaction mixture was diluted with water and the aqueous layer extracted with EA. The combined organic layers were washed with 0.1 N aqueous NaOH and 0.1 N aqueous HCl, dried over Na₂SO₄, filtered and concentrated to afford the crude title compound, which was purified by column chromatography (SiO₂; EA/heptane gradient) (1.75 g, 5.38 mmol, 77% yield).

¹H NMR (400 MHz, DMSO-d₆): δ ppm 7.12 (m, 1H), 6.98 (m, 2H), 5.33 (dd, J=8.68, 6.36 Hz, 1H), 4.36 (t, J=5.32 Hz, 1H), 4.24 (m, 1H), 3.85 (m, 1H), 3.21 (t, J=5.75 Hz, 2H), 2.86 (m, 1H), 2.69 (m, 1H), 2.18 (m, 1H), 1.91 (m, 1H), 1.76 (m, 3H), 1.32 (m, 3H), 0.95 (m, 2H)

### Example 13.2: [(3S)-3-(5-fluoro-3-pyridyl)isoxazolidin-2-yl]-[trans-4-(hydroxymethyl)cyclohexyl]methanone was Synthesized in a Similar Way to Example 13.1

### Example 13.3: Synthesis of trans-1-((4-((S)-3-(5-fluoropyridin-3-yl)isoxazolidine-2-carbonyl)cyclohexyl)methyl)-4-methyl-1H-imidazole-5-carbonitrile (Compound (4))

To a stirred solution of ((S)-3-(5-fluoropyridin-3-yl)isoxazolidin-2-yl)(trans-4-(hydroxymethyl)cyclohexyl)methanone (900 mg, 2.92 mmol) and NEt₃ (1.14 ml, 8.17 mmol) in THF (13 ml) methansulfonyl chloride (346 μl, 4.38 mmol) was added dropwise at 0° C. The resulting suspension was stirred at rt for 2 hrs. The reaction mixture was filtered, the filter cake rinsed with THF and the filtrate concentrated under reduced pressure. [trans-4-[(3S)-3-(5-fluoro-3-pyridyl)isoxazolidine-2-carbonyl]cyclohexyl]methyl methanesulfonate was obtained as pale yellow solid, which was used in the next reaction without further purification (1.10 g, 2.85 mmol, 98% yield).

To a stirred solution of 5-methyl-1H-imidazole-4-carbonitrile (64 mg, 593 μmol) in 2 ml DMF NaH (47.4 mg, 1.19 mmol) was added and stirring was continued for 10 min at rt. A solution of [trans-4-[(3S)-3-(5-fluoro-3-pyridyl)isoxazolidine-2-carbonyl]cyclohexyl]methyl methane sulfonate (crude; see above 275 mg, 711 μmol) in 2 ml DMF and KI (49.2 mg, 296 μmol) was added and the resulting suspension was stirred for 3 hrs at rt and additional 6 hrs at 50° C. followed by 16 hrs at rt. The reaction mixture was quenched with water at 0° C. and the aqueous layer extracted with MTBE, the combined organic layers were dried over Na₂SO₄, filtered and concentrated to afford the crude title compound, which was subjected to preparative reversed phase HPLC.

### Example 14: Synthesis of Compound (99) trans-1-[[4-[(3S)-3-(3-cyano-5-fluoro-phenyl)isoxazolidine-2-carbonyl]cyclohexyl]methyl]pyrrolo[3,2-b]pyridine-5-carbonitrile

Step a: Synthesis of trans-3-fluoro-5-[(3S)-2-[4-(hydroxymethyl)cyclohexanecarbonyl]isoxazolidin-3-yl]benzonitrile is performed in a similar way as in Example 13.1. using 3-fluoro-5-[(3S)-2-[4-isoxazolidin-3-yl]benzonitrile instead of (S)-3-(3,5-difluorophenyl)isoxazolidine.

Step b: Synthesis of trans-3-fluoro-5-[(3S)-2-[4-(iodomethyl)cyclohexanecarbonyl]isoxazolidin-3-yl]benzonitrile

To a solution of I₂ (7.57 g, 29.84 mmol, 6.01 ml, 1.5 eq.), PPh₃ (7.83 g, 29.84 mmol, 1.5 eq.) and imidazole (2.03 g, 29.84 mmol, 1.5 eq.) in DCM (80 ml) at 0° C. 3-fluoro-5-[(3S)-2-[trans-4-(hydroxymethyl)cyclohexanecarbonyl]isoxazolidin-3-yl]benzonitrile was added (7.60 g, 19.89 mmol, 87% purity, 1 eq.). The reaction mixture was warmed smoothly to 25° C. and stirred for 12 hrs. The reaction mixture was diluted with water (50 ml) and extracted with DCM (100 ml×3). The combined organic layers were washed with brine (50 ml), dried over Na₂SO₄, filtered and concentrated under reduced pressure. The residue was purified by silica gel column chromatography (SiO₂, petroleum ether/EA=I/O to 0/1) to give the title compound as a white solid (6.7 g, 15.15 mmol, 76% yield).

¹H NMR (400 MHz, CDCl₃): δ ppm 7.40 (s, 1H), 7.27-7.30 (m, 1H), 7.25 (d, J=1.3 Hz, 1H), 5.40 (dd, J=6.4, 8.9 Hz, 1H), 4.29 (dt, J=2.8, 7.9 Hz, 1H), 3.89 (ddd, J=6.7, 8.2, 9.6 Hz, 1H), 3.14 (dd, J=1.2, 6.1 Hz, 2H), 2.89 (dddd, J=2.9, 6.5, 9.2, 12.3 Hz, 1H), 2.74 (tt, J=3.2, 12.0 Hz, 1H), 2.30 (dddd, J=6.3, 7.6, 9.6, 12.4 Hz, 1H), 1.94-2.07 (m, 3H), 1.74-1.89 (m, 1H), 1.58-1.62 (m, 1H), 1.50-1.56 (m, 1H), 1.35-1.49 (m, 1H), 1.00-1.21 (m, 2H).

Step c: Synthesis of trans-1-[[4-[(3S)-3-(3-cyano-5-fluoro-phenyl)isoxazolidine-2-carbonyl]cyclohexyl]methyl]pyrrolo[3,2-b]pyridine-5-carbonitrile (Compound (99)).

To a solution of trans-3-fluoro-5-[(3S)-2-[4-(iodomethyl)cyclohexanecarbonyl]isoxa-zolidin-3-yl]benzonitrile (150 mg, 339.16 μmol, 1 eq.) in DMF (1.5 ml) was added K₂CO₃ (70.31 mg, 508.74 μmol, 1.5 eq.) and 1H-pyrrolo[3,2-b]pyridine-5-carbonitrile (48.55 mg, 339.16 μmol, 1 eq.). The mixture was stirred at 25° C. for 12 hrs. The reaction mixture was filtered and purified by reversed-phase HPLC (column: Phenomenex Synergi C18 150*25 mm, 10 μm, mobile phase: A: water (0.225% formic acid); B: ACN; gradient B 44%-77% in 11 minutes) to give the title compound as a white solid (48 mg, 104.39 μmol, 31% yield).

### Example 15: Synthesis of Compounds (122), (201), (228), (229), (230), (231), (232), (233), (234)

### Synthesis of (122):

### STEP a: Synthesis of 4-[[tert-butyl(dimethyl)silyl]oxymethyl]cyclohexanecarboxylic Acid

To a solution of ((1r,4r)-4-(hydroxymethyl)cyclohexane-1-carboxylic acid (1 g, 6.32 mmol) in DMF (30 mL), Et3N (2.4 mL, 17.2 mmol) and TBDMSCl (2.1 g, 13.9 mmol) were added. The mixture was stirred at rt for 3 h and then quenched with water, acidified with 1M HCl and extracted with MTBE. The organic layer was dried over Na2SO4, filtered and concentrated. The residue was diluted in DMF:H2O (20:1, 35 mL) and warmed to 70° C. then diluted with MTBE and extracted with saturated NaH4Cl, dried and purified over silica gel to give 4-[[tert-butyl(dimethyl)silyl]oxymethyl]cyclohexanecarboxylic acid (1,2 g, 60%).

### Step b: Synthesis of 5-((S)-2-((1r,4S)-4-(((tert-butyldimethylsilyl)oxy)methyl)cyclohexane-1-carbonyl)isoxazolidin-3-yl)nicotinonitrile

4-[[tert-butyl(dimethyl)silyl]oxymethyl]cyclohexanecarboxylic acid and (S)-5-(isoxazolidin-3-yl)nicotinonitrile were coupled with EDC and Oxima pure in DMF to give 5-((S)-2-((1r,4S)-4-(((tert-butyldimethylsilyl)oxy)methyl)cyclohexane-1-carbonyl)isoxazolidin-3-yl)nicotinonitrile.

### Step c: Synthesis of 5-((S)-2-((1r,4S)-4-(hydroxymethyl)cyclohexane-1-carbonyl)isoxazolidin-3-yl)nicotinonitrile

To a solution of 5-((S)-2-((1r,4S)-4-(((tert-butyldimethylsilyl)oxy)methyl)cyclohexane-1-carbonyl)isoxazolidin-3-yl)nicotinonitrile (770 mg, 1.79 mmol) in DCM (30 mL), TFA (2 mL) was added and the mixture stirred for 15 min at RT. Then the reaction was neutralized with NaHCO3 and diluted with EA and washed with water and saturated NaCl. The organic layer was dried over Na2SO4, filtered and purified by reversed phase HPLC to give 5-((S)-2-((1r,4S)-4-(hydroxymethyl)cyclohexane-1-carbonyl)isoxazolidin-3-yl)nicotinonitrile (339 mg, 60%)

### Step d: Synthesis of ((1S,4r)-4-((S)-3-(5-cyanopyridin-3-yl)isoxazolidine-2-carbonyl)cyclohexyl)methyl 4-nitrobenzenesulfonate

To a solution of 5-((S)-2-((1r,4S)-4-(hydroxymethyl)cyclohexane-1-carbonyl)isoxazolidin-3-yl)nicotinonitrile (50 mg, 160 μmol) in DCM (2.0 mL), Et3N (40 μL, 290 μmol) was added, followed by 4-nitrobenzenesulfonyl chloride (50 mg, 220 μmol). The mixture was stirred at RT overnight and then the mixture diluted with EA and extracted with water. The organic layer was concentrated and the residue filtered over silica gel, to give ((1S,4r)-4-((S)-3-(5-cyanopyridin-3-yl)isoxazolidine-2-carbonyl)cyclohexyl)methyl 4-nitrobenzenesulfonate (54 mg, 67%).

### Step e: Synthesis of trans-1-[[4-[(3S)-3-(5-cyano-3-pyridyl)isoxazolidine-2-carbonyl]cyclohexyl]methyl]-6-oxo-pyridine-3-carbonitrile (Compound (122))

To a solution of ((1S,4r)-4-((S)-3-(5-cyanopyridin-3-yl)isoxazolidine-2-carbonyl)cyclohexyl)methyl 4-nitrobenzenesulfonate (70 mg, 140 mmol) and 6-hydroxynicotinonitrile (20 mg, 210 mmol) in DMF (3 mL), Cs2CO3 (115 mg, 0.35 mmol) was added. The mixture was stirred for 2 h at 40° C. and then filtered off, and purified by HPLC to give Compound (122) (8 mg, 13.7%) 8 mg. LC/MS 1.53 min, 418.2 (Method A) Synthesis of (201):

### Step a: Synthesis of [4-[[tert-butyl(dimethyl)silyl]oxymethyl]cyclohexyl]-[(3S)-3-(6-methoxypyrazin-2-yl)isoxazolidin-2-yl]methanone

To a solution of 4-[[tert-butyl(dimethyl)silyl]oxymethyl]cyclohexanecarboxylic acid (680 mg, 2.45 mmol) in DMF (20 mL), Oxyma (900 mg, 6.33 mmol) was added, followed by EDC (1.16 g, 6.05 mmol) and NaHCO₃ (1.7 g, 20 mmol) and the mixture was stirred at rt for 1h. then rac-(3S)-3-(6-methoxypyrazin-2-yl)isoxazolidine (430 mg, 2.37 mmol) dissolved in DMF (20 mL) was added and the mixture stirred for 2 h at 40° C. Then diluted with EA, and extracted with sat. NaHCO3 (2×). The organic layer was then dried over Na2SO4, filtered and evaporated to give [4-[[tert-butyl(dimethyl)silyl]oxymethyl]cyclohexyl]-[(3S)-3-(6-methoxypyrazin-2-yl)isoxazolidin-2-yl]methanone (1.13 g, 100%), which was used as such in the following step.

### Step b: Synthesis of [4-(hydroxymethyl)cyclohexyl]-[(3S)-3-(6-methoxypyrazin-2-yl)isoxazolidin-2-yl]methanone

To a solution of [4-[[tert-butyl(dimethyl)silyl]oxymethyl]cyclohexyl]-[(3S)-3-(6-methoxypyrazin-2-yl)isoxazolidin-2-yl]methanone (1.13 mg, 2.59 mmol) in DCM (30 mL), TFA (2 mL) was added and the mixture stirred for 15 min at RT. Then the reaction was neutralized with NaHCO₃ and diluted with EA and washed with water and saturated NaCl. The organic layer was dried over Na2SO4, filtered and purified by reversed phase HPLC to give [4-(hydroxymethyl)cyclohexyl]-[(3S)-3-(6-methoxypyrazin-2-yl)isoxazolidin-2-yl]methanone (910 mg.).

### Step c: Synthesis of [4-[(3S)-3-(6-methoxypyrazin-2-yl)isoxazolidine-2-carbonyl]cyclohexyl]methyl 4-nitrobenzenesulfonate

To a solution of [4-(hydroxymethyl)cyclohexyl]-[(3S)-3-(6-methoxypyrazin-2-yl)isoxazolidin-2-yl]methanone (900 gmg, 2.83 mmol) in DCM (mL), ET3N was added, followed by 4-nitrobenzenesulfonyl chloride (950 mg, 4.28 mmol). The mixture was stirred at rt overnight and then diluted with EA and extracted with water. The organic layer was filtered over a silica gel path and evaporated, to give [4-[(3S)-3-(6-methoxypyrazin-2-yl)isoxazolidine-2-carbonyl]cyclohexyl]methyl 4-nitrobenzenesulfonate (1.22 g, 85%), which was used without further purification.

### Step d: Synthesis of 1-[[4-[(3S)-3-(6-methoxypyrazin-2-yl)isoxazolidine-2-carbonyl]cyclohexyl]methyl]indazole-6-carboxamide (Compound (201))

A solution of [4-[(3S)-3-(6-methoxypyrazin-2-yl)isoxazolidine-2-carbonyl]cyclohexyl]methyl 4-nitrobenzenesulfonate (100 mg, 0.119 mmol) and 1H-indazole-6-carboxamide (50 mg, 0.31 mmol) and Cs2CO3 (160 mg, 0.49 mmol) in DMF (4 mL) was stirred at rt for 2 h. The mixture was filtered off and purified via reversed phase chromatography to give 1-[[4-[(3S)-3-(6-methoxypyrazin-2-yl)isoxazolidine-2-carbonyl]cyclohexyl]methyl]indazole-6-carboxamide (2 mg, 2%).

### Synthesis of Compounds (228), (229), (230), (231), (232), (233), (234),

STEP 1: Synthesis of trans-4-[(6-carbamoylindazol-1-yl)methyl]cyclohexanecarboxylic acid from trans-4-[(6-cyanoindazol-1-yl)methyl]cyclohexanecarboxylic acid as synthesized in Example 3.5 performed as detailed in Example 9.1.

STEP 2a: Synthesis of trans-1-[[4-[(3S)-3-(6-methoxypyrazin-2-yl)isoxazolidine-2-carbonyl]cyclohexyl]methyl]indazole-6-carboxamide (Compound (201)) is performed as in STEP 5c of Example 1.4, but starting from trans-4-[(6-carbamoylindazol-1-yl)methyl]cyclohexanecarboxylic acid and (3S)-3-(6-methoxypyrazin-2-yl)isoxazolidine.

STEP 2b: Synthesis of trans-2-[[4-[(3S)-3-pyrazin-2-ylisoxazolidine-2-carbonyl]cyclohexyl]methyl]indazole-6-carboxamide (Compound (228)) is performed as in STEP 5c of Example 1.4, but starting from trans-4-[(6-carbamoylindazol-1-yl)methyl]cyclohexanecarboxylic acid and (3S)-3-pyrazin-2-ylisoxazolidine.

STEP 2c: Synthesis of trans-1-[[4-[(3S)-3-pyrazin-2-ylisoxazolidine-2-carbonyl]cyclohexyl]methyl]indazole-6-carboxamide (Compound (229)) is performed as in STEP 5c of Example 1.4, but starting from trans-4-[(6-carbamoylindazol-1-yl)methyl]cyclohexanecarboxylic acid and (3S)-3-pyrazin-2-ylisoxazolidine.

STEP 2d: Synthesis of trans-1-[[4-[(3S)-3-(2-pyridyl)isoxazolidine-2-carbonyl]cyclohexyl]methyl]indazole-6-carboxamide (Compound (230)) is performed as in STEP 5c of Example 1.4, but starting from trans-4-[(6-carbamoylindazol-1-yl)methyl]cyclohexanecarboxylic acid and (3S)-3-pyrazin-2-ylisoxazolidine.

STEP 2e: Synthesis of trans-1-[[4-[(3S)-3-(4-cyano-2-pyridyl)isoxazolidine-2-carbonyl]cyclohexyl]methyl]indazole-6-carboxamide (Compound (231)) is performed as in STEP 5c of Example 1.4, but starting from trans-4-[(6-carbamoylindazol-1-yl)methyl]cyclohexanecarboxylic acid and (3S)-3-(4-cyano-2-pyridyl)isoxazolidine.

STEP 2f: Synthesis of trans-2-[[4-[(3S)-3-(4-cyano-2-pyridyl)isoxazolidine-2-carbonyl]cyclohexyl]methyl]indazole-6-carboxamide (Compound (232)) is performed as in STEP 5c of Example 1.4, but starting from trans-4-[(6-carbamoylindazol-1-yl)methyl]cyclohexanecarboxylic acid and (3S)-3-(4-cyano-2-pyridyl)isoxazolidine.

STEP 2g: Synthesis of trans-2-[[4-[(3S)-3-(2-methylthiazol-4-yl)isoxazolidine-2-carbonyl]cyclohexyl]methyl]indazole-6-carboxamide (Compound (233)) is performed as in STEP 5c of Example 1.4, but starting from trans-4-[(6-carbamoylindazol-1-yl)methyl]cyclohexanecarboxylic acid and (3S)-3-(2-methylthiazol-4-yl)isoxazolidine.

STEP 2g: Synthesis of trans-1-[[4-[(3S)-3-(2-methylthiazol-4-yl)isoxazolidine-2-carbonyl]cyclohexyl]methyl]indazole-6-carboxamide (Compound (234)) is performed as in STEP 5c of Example 1.4, but starting from trans-4-[(6-carbamoylindazol-1-yl)methyl]cyclohexanecarboxylic acid and (3S)-3-(2-methylthiazol-4-yl)isoxazolidine.

### Example 16: Synthesis of Compounds (256), (258), (312), (313), (314)

### Example 16.1: Synthesis of Compound (256)

In a vial under an Ar atmosphere NiBr₂ ethylene glycol dimethyl ether complex (1.5 mg, 5 μmol) and 4,4′-di-tert-butyl-2,2′-bipyridine (1.4 mg, 5 μmol) were suspended in 1 ml dioxane. The suspension was sonicated at 40° C. in an ultrasound bath for 20 min to form a catalyst suspension.

(3-bromo-5-fluoro-phenyl)methanol (52 mg, 0.25 mmol), 3-[(3S)-2-trans-[4-(bromomethyl)cyclohexanecarbonyl]isoxazolidin-3-yl]-5-fluoro-benzonitrile (100 mg, 0.25 mmol), and cobalt(II)phthalocyanine (CAS 3317-67-7; 7.2 mg, 12 μmol) were placed in a vial under an Ar atmosphere. The catalyst suspension was added and Ar was bubbled through the resulting suspension for 10 min. Tetrakis(dimethylamino)ethylene (TDAE) (51 mg, 0.25 mmol) was added and the vial sealed and heated to 80° C. for 24 hrs. The mixture was cooled to rt and the resulting residue partitioned between EA and 0.1 N HCl. The aqueous layer was extracted with EA and the combined organic layers were dried over Na₂SO₄, filtered and concentrated to afford the crude title compound, filtered and subjected to preparative reversed phase HPLC (20 mg, 45 μmol, 18% yield).

### Example 16.2: Synthesis of Compound (258)

In a vial under an Ar atmosphere NiBr₂ ethylene glycol dimethyl ether complex (10.0 mg, 32 μmol) and 4,4′-di-tert-butyl-2,2′-bipyridine (8.8 mg, 32 μmol) were suspended in 2.8 ml dioxane. The suspension was sonicated at 40° C. in an ultrasound bath for 15 min to form a catalyst suspension.

4-bromo-1-methyl-triazole (64 mg, 0.4 mmol), 3-[(3S)-2-[trans-4-(bromomethyl)cyclohexane carbonyl]isoxazolidin-3-yl]-5-fluoro-benzonitrile (130 mg, 0.33 mmol), LiOH (16.0 mg, 0.66 mmol), tris(trimethylsilyl)silane (82 mg, 0.33 mmol, 0.10 ml) and [Ir(dF(Me)ppy)₂(dtbbpy)]PF₆ (1.7 mg, 1.6 μmol) were placed in a vial under an Ar atmosphere. The catalyst suspension was added and Ar was bubbled through (the resulting suspension for 10 min. The vial was sealed and irradiated in a PennOC photoreactor M1 (100% light intensity) for 24 hrs.

Volatile components were removed under reduced pressure and the resulting residue was partitioned between EA and water. The aqueous layer extracted with EA and the combined organic layers were dried over Na₂SO₄, filtered and concentrated to afford the crude title compound, which was subjected to preparative reversed phase HPLC (14 mg, 36 μmol, 11% yield).

### Example 16.2: Synthesis of Compound (312), (313)

In a bottom flask were introduced (1,2-Dimethoxyethane)nickel dibromide (4,829 mg, 0,05000 equiv.) and 4,4′-di-tert-butyl-2,2′-bipyridine (4,074 mg, 0,05000 equiv.) and 1,4-Dioxane (100 mass %, 2.60 mL, 2675 mg, 100.0 equiv) was added. The suspension was sonicated in a sonication bad at 40° C. for 15 min. The yellow suspension turned into a light green solution.

In a separate reaction vial were introduced 3-[(3S)-2-[4-(bromomethyl)cyclohexanecarbonyl]isoxazolidin-3-yl]-5-fluoro-benzonitrile (A, 100 mass %, 120 mg, 1,000 equiv), 4-BROMO-2,6-DIMETHYLPYRIMIDINE (B, 100 mass %, 68.13 mg, 1,200 equiv), lithium hydroxide (14.54 mg, 2,000 equiv.) and [IR(DF(ME)PPY)2(DTBBPY)]PF6 (100 mass %, 6,156 mg, 0,02000 equiv). Then, solution A was added to this mixture followed by tris(trimethylsilyl)silane (100 mass %, 0,112 mL, 90.58 mg, 1,200 equiv) and the solution was purged with Ar for 15 min. The reaction vessel was then placed in a Merck Photoreactor for 18h (fan 4000 rpm, stir 400 rpm, LED 100%). After this the solvent was evaporated in a rotavapor and the residue taken up in Ethyl acetate and washed with water. The organic layer was dried and concentrated in vacuo. The residue was dissolved in DMF and purified by HPLC (C, 100 mass %, 13.7 mg, 0.107 equiv, 10.7%).

Synthesis of 3-[(3S)-2-[4-[(2,6-dimethylpyrimidin-4-yl)methyl]cyclohexanecarbonyl]isoxazolidin-3-yl]-5-fluoro-benzonitrile is performed as detailed here-above for compound (312) but starting from [4-(bromomethyl)cyclohexyl]-[(3S)-3-(3,5-difluorophenyl)isoxazolidin-2-yl]methanone (1,000 eq., 120 mg) and 4-bromo-6-methyl-pyrimidine (B, 100 mass %, 74.87 mg, 1,400 equiv).

### Example 17: Synthesis of Compound (314), (342), (345), (367), (368) and (383)

### Example 17:1 Synthesis of [4-[(8-methoxy-2-methyl-[1,2,4]triazolo[1,5-a]pyridin-6-yl)methyl]cyclohexyl]-[(3S)-3-pyrazin-2-yl-1,2-oxazolidin-2-yl]methanone (314)

STEP 1: To a solution of 5-bromo-3-methoxy-pyridin-2-amine (5 g, 24.63 mmol, 1 eq) in DMF (25 mL) was added 1,1-dimethoxy-N,N-dimethyl-ethanamine (9.84 g, 73.88 mmol, 10.80 mL, 3 eq). The mixture was stirred at 130° C. for 12 hr. LCMS showed 5-bromo-3-methoxy-pyridin-2-amine was consumed completely and one main peak with desired mass was detected. The reaction mixture was concentrated under reduced pressure to remove solvent. N′-(5-bromo-3-methoxy-2-pyridyl)-N,N-dimethyl-acetamidine (7.54 g, crude) was obtained as a black oil and used into the next step without further purification. LCMS: m/z 272.1 [M+1]+.

STEP 2: To a solution of N′-(5-bromo-3-methoxy-2-pyridyl)-N,N-dimethyl-acetamidine (7.54 g, 27.71 mmol, 1 eq) and PYRIDINE (10.96 g, 138.53 mmol, 11.18 mL, 5 eq) in MeOH (180 mL) was added amino hydrogen sulfate (6.27 g, 55.41 mmol, 2 eq) at 0° C. The mixture was stirred at 25° C. for 16 hr. LC-MS showed desired compound (Rt=0.642 min). The reaction mixture was concentrated under reduced pressure, diluted with H₂O 150 mL and extracted with ethyl acetate 450 mL (150 mL*3). The combined organic layers were washed with saturated salt solution 150 mL (150 mL*1), dried over Na2SO4, filtered and concentrated under reduced pressure to give a residue. The residue was purified by column chromatography (SiO2, Petroleum ether/Ethyl acetate=10/1 to 3/1). 6-bromo-8-methoxy-2-methyl-[1,2,4]triazolo[1,5-a]pyridine (3.4 g, 14.05 mmol, 50.69% yield) was obtained as a white solid.

LCMS: m/z 242.1 [M+1]+

¹H NMR: (CHLOROFORM-d, 400 MHz): δ ppm 8.29-8.26 (m, 1H), 6.83 (s, 1H), 4.04-4.03 (m, 3H), 2.59-2.57 (m, 3H)

STEP 3: To an 40 mL vial equipped with a stir bar was added 6-bromo-8-methoxy-2-methyl-[1,2,4]triazolo[1,5-a]pyridine (750 mg, 3.10 mmol, 1 eq), methyl 4-(bromomethyl)cyclohexanecarboxylate (946.99 mg, 4.03 mmol, 1.3 eq), TTMSS (770.41 mg, 3.10 mmol, 955.84 uL, 1 eq), Na₂CO₃ (656.77 mg, 6.20 mmol, 2 eq), Ir[dF(CF3)ppy]2(dtbpy)(PF6) (34.76 mg, 30.98 μmol, 0.01 eq), NiCl2·dtbbpy (6.17 mg, 15.49 μmol, 0.005 eq) in DME (30 mL). The vial was sealed and placed under nitrogen was added. The reaction was stirred and irradiated with a 34 W blue LED lamp (7 cm away), with cooling fan to keep the reaction temperature at 25° C. for 14 h. (4 batches in parallel). LC-MS showed 6-bromo-8-methoxy-2-methyl-[1,2,4]triazolo[1,5-a]pyridine was consumed completely and one main peak (Rt=0.752) with desired m/z was detected. The reaction mixture was filtered and concentrated under reduced pressure to give a residue. The crude product was purified by reversed-phase HPLC (0.1% FA condition: flow: 100 mL/min; gradient: from 95% H2O (0.1% FA)/5% ACN to 45% H2O (0.1% FA)/55% ACN in 30 min; 45% H2O (0.1% FA)/55% ACN to 100% H2O (0.1% FA) in 20 min; column: Welch Ultimate XB_C18, 20-40 μm, 120 A). methyl 4-[(8-methoxy-2-methyl-[1,2,4]triazolo[1,5-a]pyridin-6-yl)methyl]cyclohexanecarboxylate (1.33 g, 4.09 mmol, 32.98% yield, 97.54% purity) was obtained as a white solid.

LCMS: m/z 318.2 [M+1]⁺

¹H NMR: (CHLOROFORM-d, 400 MHz): δ ppm 8.18 (s, 1H), 7.92 (s, 1H), 6.64-6.56 (m, 1H), 4.02 (s, 3H), 4.07-3.98 (m, 1H), 3.66 (s, 3H), 3.70-3.62 (m, 1H), 2.61 (br s, 1H), 2.59 (s, 2H), 2.52 (d, J=7.1 Hz, 2H), 2.26 (tt, J=3.4, 12.2 Hz, 1H), 1.99 (br d, J=11.9 Hz, 2H), 1.80 (br d, J=11.2 Hz, 2H), 1.56 (ft, J=7.5, 11.3 Hz, 1H), 1.41 (dq, J=3.0, 12.9 Hz, 3H), 1.12-0.96 (m, 2H)

STEP 4: To a solution of methyl 4-[(8-methoxy-2-methyl-[1,2,4]triazolo[1,5-a]pyridin-6-yl)methyl]cyclohexanecarboxylate in THF (1 mL) was added LiOH·H2O (1 M, 19.38 mL, 5 eq). The mixture was stirred at 25° C. for 2 hr. LC-MS showed methyl 4-[(8-methoxy-2-methyl-[1,2,4]triazolo[1,5-a]pyridin-6-yl)methyl]cyclohexanecarboxylate was consumed completely and desired mass (Rt=0.837) was detected. The reaction mixture was adjusted to pH=6 with 1 N HCl aqueous solution 20 mL, the precipitate was collected through filtration and dried in vacuo. 4-[(8-methoxy-2-methyl-[1,2,4]triazolo[1,5-a]pyridin-6-yl)methyl]cyclohexanecarboxylic acid (0.65 g, crude) was obtained as a white solid.

LCMS: m/z 304.2 [M+1]⁺

¹H NMR: (CHLOROFORM-d, 400 MHz): δ ppm 7.95-7.89 (m, 1H), 7.33-7.22 (m, 3H), 6.65-6.60 (m, 1H), 4.04 (s, 3H), 2.63 (s, 3H), 2.57-2.52 (m, 2H), 2.37-2.25 (m, 1H), 2.09-2.00 (m, 3H), 1.86-1.79 (m, 3H), 1.50-1.37 (m, 3H), 1.11-0.98 (m, 2H)

SFC: 99.086%

STEP 5a: To a solution of 4-[(8-methoxy-2-methyl-[1,2,4]triazolo[1,5-a]pyridin-6-yl)methyl]cyclohexanecarboxylic acid (0.1 g, 329.65 μmol, 1 eq), HATU (188.01 mg, 494.47 μmol, 1.5 eq) and DIEA (213.02 mg, 1.65 mmol, 287.09 uL, 5 eq) in DMF (1 mL) was added (3S)-3-pyrazin-2-ylisoxazolidine (68.04 mg, 362.61 μmol, 1.1 eq, HCl). The mixture was stirred at 25° C. for 12 hr. LC-MS showed 4-[(8-methoxy-2-methyl-[1,2,4]triazolo[1,5-a]pyridin-6-yl)methyl]cyclohexanecarboxylic acid was consumed completely and desired mass (Rt=0.628) was detected. The resulting product Synthesis of [4-[(8-methoxy-2-methyl-[1,2,4]triazolo[1,5-a]pyridin-6-yl)methyl]cyclohexyl]-[(3S)-3-pyrazin-2-yl-1,2-oxazolidin-2-yl]methanone (Compound 314) was filtered to remove the insoluble. The residue was purified by prep-HPLC (FA condition).

STEP 5b: Synthesis of trans-[(3S)-3-(5-fluoro-6-methylpyridin-3-yl)-1,2-oxazolidin-2-yl]-[4-[(8-methoxy-2-methyl-[1,2,4]triazolo[1,5-a]pyridin-6-yl)methyl]cyclohexyl]methanone (Compound 342) is performed as in STEP 5a here-above but starting from (3S)-3-(5-Fluoro-6-methyl-3-pyridyl)isoxazolidine instead of (3S)-3-pyrazin-2-ylisoxazolidine

Chiral SFC: RT 1.75 min (100%, Method Z)

STEP 5c: Synthesis of trans-[(3S)-3-(5-fluoropyridin-3-yl)-1,2-oxazolidin-2-yl]-[4-[(8-methoxy-2-methyl-[1,2,4]triazolo[1,5-a]pyridin-6-yl)methyl]cyclohexyl]methanone (Compound 345) is performed as in STEP 5a here-above but starting from 3-(5-Fluoro-3-pyridyl)isoxazolidine instead of (3S)-3-pyrazin-2-ylisoxazolidine

Chiral SFC: RT 1.29 min (100%, Method X)

STEP 5d: Synthesis of trans-[4-(8-Methoxy-2-methyl-[1,2,4]triazolo[1,5-a]pyridin-6-ylmethyl)-cyclohexyl]-[(S)-3-(2-methyl-thiazol-4-yl)-isoxazolidin-2-yl]-methanone (Compound 367) is performed as in STEP 5a here-above but starting from (3S)-3-(2-Methylthiazol-4-yl)isoxazolidine instead of (3S)-3-pyrazin-2-ylisoxazolidine

STEP 5d: Synthesis of trans-[4-(8-Methoxy-2-methyl-[1,2,4]triazolo[1,5-a]pyridin-6-ylmethyl)-cyclohexyl]-[(S)-3-(6-methyl-pyridin-3-yl)-isoxazolidin-2-yl]-methanone (Compound 368) is performed as in STEP 5a here-above but starting from (3S)-3-(6-Methyl-3-pyridyl)isoxazolidine instead of (3S)-3-pyrazin-2-ylisoxazolidine

Chiral SFC: RT 1.24 min (100%, Method AH)

STEP 5d: Synthesis of trans-[4-[(8-methoxy-2-methyl-[1,2,4]triazolo[1,5-a]pyridin-6-yl)methyl]cyclohexyl]-[(3S)-3-(5-methylpyridin-3-yl)-1,2-oxazolidin-2-yl]methanone (Compound 383) is performed as in STEP 5a here-above but starting from (3S)-3-(5-methyl-3-pyridyl)isoxazolidine instead of (3S)-3-pyrazin-2-ylisoxazolidine

Chiral SFC: RT 1.33 min (100%, Method X)

### Example 18: Synthesis of Compounds (332), (339), (354), (356), (366) and (377)

STEP 1: Synthesis of methyl trans-4-[(2,5-dimethyl-4-pyridyl)methyl]cyclohexanecarboxylate—Compound of formula (IIIb) in Scheme 3—is performed as in example 8.27 here-above

STEP 2: Synthesis of trans-4-[(2,5-dimethyl-4-pyridyl)methyl]cyclohexanecarboxylate—Compound (IIa) in Scheme 1—in a similar way as performed in Example 1.1, but using methyl trans-4-[(2,5-dimethyl-4-pyridyl)methyl]cyclohexanecarboxylate instead of trans-4-(benzimidazol-1-ylmethyl)cyclohexanecarboxylate

STEP 3a: Synthesis of trans-[4-[(2,5-dimethylpyridin-4-yl)methyl]cyclohexyl]-[(3S)-3-(5-fluoro-6-methylpyridin-3-yl)-1,2-oxazolidin-2-yl]methanone (Compound (332)) is performed as in STEP 5c of Example 1.4, but starting from trans-4-[(2,5-dimethyl-4-pyridyl)methyl]cyclohexanecarboxylate and (3S)-3-(5-Fluoro-6-methyl-3-pyridyl)isoxazolidine instead of trans-4-[(5-cyano-6-fluoro-benzimidazol-1-yl)methyl]cyclohexanecarboxylic acid and 3-fluoro-5-[(3S)-isoxazolidin-3-yl]benzonitrile.

Chiral SFC: RT 1.04 min (100%, Method AE)

STEP 3b: Synthesis of trans-[4-[(2,5-dimethylpyridin-4-yl)methyl]cyclohexyl]-[(3S)-3-(5-methylpyrazin-2-yl)-1,2-oxazolidin-2-yl]methanone (Compound (339)) is performed as in STEP 3a here-above, but starting from (3S)-3-(5-methylpyrazin-2-yl)isoxazolidine instead of (3S)-3-(5-Fluoro-6-methyl-3-pyridyl)isoxazolidine

Chiral SFC: RT 1.68 min (100%, Method AE)

STEP 3c: Synthesis of trans-[4-(2,5-Dimethyl-pyridin-4-ylmethyl)-cyclohexyl]-[(S)-3-(6-methyl-pyridin-3-yl)-isoxazolidin-2-yl]-methanone (Compound (354)) is performed as in STEP 3a here-above, but starting from (3S)-3-(6-Methyl-3-pyridyl)isoxazolidine instead of (3S)-3-(5-Fluoro-6-methyl-3-pyridyl)isoxazolidine

Chiral SFC: RT 1.17 min (100%, Method X)

STEP 3d: Synthesis of trans-[4-(2,5-Dimethyl-pyridin-4-ylmethyl)-cyclohexyl]-((S)-3-pyrazin-2-yl-isoxazolidin-2-yl)-methanone (Compound (356)) is performed as in STEP 3a here-above, but starting (3S)-3-pyrazin-2-ylisoxazolidine instead of (3S)-3-(5-Fluoro-6-methyl-3-pyridyl)isoxazolidine

STEP 3e: Synthesis of trans-[4-(2,5-Dimethyl-pyridin-4-ylmethyl)-cyclohexyl]-[(S)-3-(2-methyl-thiazol-4-yl)-isoxazolidin-2-yl]-methanone (Compound (366)) is performed as in STEP 3a here-above, but starting (3S)-3-(2-Methylthiazol-4-yl)isoxazolidine instead of (3S)-3-(5-Fluoro-6-methyl-3-pyridyl)isoxazolidine

STEP 3f: Synthesis of trans-[4-[(2,5-dimethylpyridin-4-yl)methyl]cyclohexyl]-[(3S)-3-(2-methyl-1,3-oxazol-4-yl)-1,2-oxazolidin-2-yl]methanone (Compound (377)) is performed as in STEP 3a here-above, but starting (3S)-3-(2-Methyloxazol-4-yl)isoxazolidine instead of (3S)-3-(5-Fluoro-6-methyl-3-pyridyl)isoxazolidine

Chiral SFC: RT 0.92 min (100%, Method AE)

### Example 19: Synthesis of Cpds (381) and (382)

Step 1. Synthesis of methyl 4-[(8-chloro-2-methyl-[1,2,4]triazolo[1,5-a]pyridin-6-yl)methyl]cyclohexanecarboxylate. The compound of formula (IIIb) in Scheme 3, is performed in a similar way as in example 8.2—Step 1 here-above using 6-bromo-8-chloro-2-methyl-[1,2,4]triazolo[1,5-a]pyridine (1 g, 4.06 mmol) and methyl trans-4-(bromomethyl)cyclohexanecarboxylate (1.14 g, 4.87 mmol) to give the above intermediate (214 mg) as a white solid.

LCMS, RT 0.950 min m/z=322.1 [M+H]+ (Method Q)

¹H NMR (400 MHz, CDCl₃) δ=8.18 (s, 1H), 7.37 (d, J=1.2 Hz, 1H), 3.66 (s, 3H), 2.62 (s, 3H), 2.54 (d, J=7.2 Hz, 2H), 2.31-2.20 (m, 1H), 2.05-1.96 (m, 2H), 1.87-1.74 (m, 2H), 1.60-1.51 (m, 1H), 1.49-1.35 (m, 2H), 1.10-0.94 (m, 2H).

Step 2. Synthesis of trans-4-[(8-chloro-2-methyl-[1,2,4]triazolo[1,5-a]pyridin-6-yl)methyl]cyclohexanecarboxylic acid. The compound of formula (IIa) in Scheme 1, is performed in a similar way as in example 1.1 here-above, but starting with methyl 4-[(8-chloro-2-methyl-[1,2,4]triazolo[1,5-a]pyridin-6-yl)methyl]cyclohexanecarboxylate (194 mg, 0.60 mmol) to afford the desired intermediate (180 mg, 96%) as a white solid.

LCMS: RT 0.524 min, m/z=308.2 [M+H]⁺ (Method C)

Step 3.1. Synthesis of [4-[(8-chloro-2-methyl-[1,2,4]triazolo[1,5-a]pyridin-6-yl)methyl]cyclohexyl]-[(3S)-3-(5-fluoro-6-methylpyridin-3-yl)-1,2-oxazolidin-2-yl]methanone (Compound 381).

This compound was synthesized in a similar manner as step 5c of example 1.4 starting with 4-[(8-chloro-2-methyl-[1,2,4]triazolo[1,5-a]pyridin-6-yl)methyl]cyclohexanecarboxylic acid (90 mg, 0.28 mmol) and (3S)-3-(5-Fluoro-6-methyl-3-pyridyl)isoxazolidine (219 mg, 29% purity) to give Compound 381 (43.06 mg, 32%) as a white solid.

Chiral SFC: RT 1.362 min, 99.99% ee (Method Z)

Step 3.2. Synthesis of [4-[(8-chloro-2-methyl-[1,2,4]triazolo[1,5-a]pyridin-6-yl)methyl]cyclohexyl]-[(3S)-3-(5-fluoropyridin-3-yl)-1,2-oxazolidin-2-yl]methanone (Compound 382). This compound was synthesized in a similar manners step 5c of example 1.4 starting with (3S)-3-(5-Fluorol-3-pyridyl)isoxazolidine (199 mg, 29% purity) and 4-[(8-chloro-2-methyl-[1,2,4]triazolo[1,5-a]pyridin-6-yl)methyl]cyclohexanecarboxylic acid (90 mg, 0.28 mmol) to give Compound 382 (63 mg, 46%) as a white solid.

Chiral SFC: RT 1.394 min, 99.99% ee, (Method X)

### Example 20. Synthesis of (391)

### Step 1. Synthesis of methyl 4-[[6-(tert-butoxycarbonylamino)-3-pyridyl]methyl]cyclohexanecarboxylate

The compound of formula (IIIb) in Scheme 3, is performed in a similar way as in example 8.2—Step 1 here-above using methyl trans-4-(bromomethyl)cyclohexanecarboxylate (5.6 g, 23.8 mmol) and tert-butyl N-(5-bromo-2-pyridyl)carbamate (5 g, 12.3 mmol) to give the desired intermediate (3.44 g, 54%) as a yellow solid. ¹H NMR (400 MHz, DMSO-d₆) δ=9.64 (s, 1H), 8.02 (d, J=1.2 Hz, 1H), 7.69 (d, J=8.4 Hz, 1H), 7.51 (dd, J=8.4, 1.2 Hz, 1H), 3.56 (s, 3H), 2.40 (d, J=6.8 Hz, 2H), 2.24-2.18 (m, 1H), 1.86-1.80 (m, 2H), 1.66-1.57 (m, 2H), 1.51-1.43 (m, 1H), 1.46 (s, 9H), 1.31-1.20 (m, 2H), 1.02-0.89 (m, 2H).

### Step 2. Synthesis of methyl 4-[(6-amino-3-pyridyl)methyl]cyclohexanecarboxylate

A solution of methyl 4-[[6-(tert-butoxycarbonylamino)-3-pyridyl]methyl]cyclohexanecarboxylate (2.00 g, 0.00941 mol, 1.00 eq.) in HCl/dioxane (2 M, 15 mL) was stirred at 25° C. for 5 h. The reaction mixture was filtered and washed with dioxane to afford methyl 4-[(6-amino-3-pyridyl)methyl]cyclohexanecarboxylate (1.28 g, 0.00484 mol, 84.4% yield) as a white solid.

¹H NMR (400 MHz, CD₃OD) δ=7.83 (dd, J=9.2, 1.2 Hz, 1H), 7.63 (d, J=1.2 Hz, 1H), 6.99 (d, J=9.2 Hz, 1H), 3.64 (s, 3H), 2.45 (d, J=7.2 Hz, 2H), 2.34-2.20 (m, 1H), 2.01-1.90 (m, 2H), 1.81-1.70 (m, 2H), 1.58-1.47 (m, 1H), 1.44-1.33 (m, 2H), 1.11-0.96 (m, 2H).

### Step 3. Synthesis of methyl 4-[[6-(ethoxycarbonylcarbamothioylamino)-3-pyridyl]methyl]cyclohexanecarboxylate

To a solution of methyl 4-[(6-amino-3-pyridyl)methyl]cyclohexanecarboxylate (1.28 g, 0.00488 mol, 1.00 eq.) in dioxane (15 mL) was added ethyl N-(thioxomethylene)carbamate (0.640 g, 0.00488 mol, 1.00 eq.) and DIEA (4.25 mL, 0.0244 mol, 5.00 eq.). And the resulting mixture was stirred at 25° C. for 16 h. The reaction mixture was directly concentrated under reduced pressure to give a residue. The residue was washed with EtOAc to afford methyl 4-[[6-(ethoxycarbonylcarbamothioylamino)-3-pyridyl]methyl]cyclohexanecarboxylate (1.40 g, 0.00369 mol, 71.0% yield) as a yellow solid.

LCMS: RT 0.649 min, m/z=380.2 [M+H]+ (Method C).

### Step 4. Synthesis of methyl trans-4-[(2-amino-[1,2,4]triazolo[1,5-a]pyridin-6-yl)methyl]cyclohexanecarboxylate

To a solution of methyl 4-[[6-(ethoxycarbonylcarbamothioylamino)-3-pyridyl]methyl]cyclohexanecarboxylate (1.40 g, 0.00369 mol, 1.00 eq.) and NH₂OH·HCl (1.27 g, 0.0184 mol, 5.00 eq.) in EtOH/MeOH (v/v=1/1, 20 mL) was added DIEA (1.93 mL, 0.0111 mol, 3.00 eq.). And the mixture was stirred at 60° C. for 3 h. The reaction mixture was concentrated under reduced pressure to give a residue. The residue was washed with EtOAc (8 mL) to afford methyl trans-4-[(2-amino-[1,2,4]triazolo[1,5-a]pyridin-6-yl)methyl]cyclohexanecarboxylate (688 mg, 0.00239 mol, 64.7% yield) as a yellow solid.

LCMS: RT 0.494 min, m/z=289.2 [M+H]+(Method C)

### Step 5. Synthesis of 4-[(2-amino-[1,2,4]triazolo[1,5-a]pyridin-6-yl)methyl]cyclohexanecarboxylic Acid

LCMS: RT 0.460 min, m/z=275.2 [M+H]⁺ (Method C)

¹H NMR (400 MHz, CD₃OD) δ=8.21 (s, 1H), 7.43-7.35 (m, 1H), 7.33-7.25 (m, 1H), 2.53 (d, J=7.2 Hz, 2H), 2.27-2.16 (m, 1H), 1.97 (d, J=11.6 Hz, 2H), 1.77 (d, J=11.6 Hz, 2H), 1.58-1.51 (m, 1H), 1.38 (qd, J=12.8, 2.8 Hz, 2H), 1.11-0.97 (m, 2H).

Step 6. Synthesis of [4-[(2-amino-[1,2,4]triazolo[1,5-a]pyridin-6-yl)methyl]cyclohexyl]-[(3S)-3-(6-methylpyridin-3-yl)-1,2-oxazolidin-2-yl]methanone (Compound 391)—This compound was synthesized in a similar manner as step 5c of example 1.4 starting with (3S)-3-(6-Methyl-3-pyridyl)isoxazolidine (579 mg, 25% purity) and 4-[(2-amino-[1,2,4]triazolo[1,5-a]pyridin-6-yl)methyl]cyclohexanecarboxylic acid (220 mg, 0.8 mmol) to afford Compound (391) (102.8 mg, 30%) as an off-white solid.

Chiral SFC: RT 1.627 min, 99.99% ee, (Method P)

### Example 21: Synthesis of (393)

Step 1. Synthesis of methyl trans-4-[(2,4-dimethylpyrimidin-5-yl)methyl]cyclohexanecarboxylate. The compound of formula (IIIb) in Scheme 3, is performed in a similar way as in example 8.2—Step 1 here-above but starting with methyl trans-4-(bromomethyl)cyclohexanecarboxylate (2.7 g, 11.5 mmol) and 5-bromo-2,4-dimethyl-pyrimidine (1.65 g, 8.88 mmol) to give the desired intermediate (1.15 g, 47%) as a yellow solid.

1H NMR (400 MHz, CDCl3) δ=8.25 (s, 1H), 3.66 (s, 3H), 2.68 (s, 3H), 2.49-2.45 (m, 5H), 2.32-2.23 (m, 1H), 2.03-1.96 (m, 2H), 1.79-1.74 (m, 2H), 1.56-1.47 (m, 1H), 1.39 (qd, J=12.6, 3.2 Hz, 2H), 1.11-0.99 (m, 2H).

Step 2. Synthesis of trans-4-[(2,4-dimethylpyrimidin-5-yl)methyl]cyclohexanecarboxylic acid. This Compound of formula (IIa) in Scheme 1, is performed in a similar way as in example 1.1 here-above, but starting with methyl trans-4-[(2,4-dimethylpyrimidin-5-yl)methyl]cyclohexanecarboxylate (1.15 g, 4.1 mmol) to give the desired intermediate (1.0 g, 97%) as a white solid.

1H NMR (400 MHz, CDCl3) δ=8.30 (s, 1H), 2.68 (s, 3H), 2.51-2.45 (m, 5H), 2.34-2.24 (m, 1H), 2.07-2.01 (m, 2H), 1.83-1.72 (m, 2H), 1.57-1.47 (m, 1H), 1.46-1.35 (m, 2H), 1.13-0.95 (m, 2H).

Step 3. Synthesis of [4-[(2,4-dimethylpyrimidin-5-yl)methyl]cyclohexyl]-[(3S)-3-(5-fluoro-6-methylpyridin-3-yl)-1,2-oxazolidin-2-yl]methanone (Compound (393))

This compound was synthesized in a similar manner step 5c of example 1.4 starting with 4-[(2,4-dimethylpyrimidin-5-yl)methyl]cyclohexanecarboxylic acid (230 mg, 0.926 mmol) and (3S)-3-(5-Fluoro-6-methyl-3-pyridyl)isoxazolidine (169 mg, 0.92 mmol) to give Compound 393 (146.07 mg, 38%) as a white solid.

Chiral SFC. RT 1.325 min (Method M)

### Example 22: Synthesis of (399)

Step 1. Synthesis of methyl 4-[[2-methyl-4-(trifluoromethyl)pyrimidin-5-yl]methyl]cyclohexanecarboxylate. The compound of formula (IIIb) in Scheme 3, is performed in a similar way as in example 8.2—Step 1 here-above but starting with methyl trans-4-(bromomethyl)cyclohexanecarboxylate (1.27 mg, 5.39 mmol) and 5-bromo-2-methyl-4-(trifluoromethyl)pyrimidine (1 g, 4.15 mmol) to give the desired intermediate (443 mg, 55%) as a yellow oil.

LCMS: RT 0.612 min, m/z=317.2 [M+H]+ (Method C).

¹H NMR (400 MHz, CDCl₃) δ=8.64 (s, 1H), 3.41 (s, 3H), 2.79 (s, 3H), 2.66 (d, J=6.8 Hz, 2H), 2.30-2.23 (m, 1H), 2.03-1.97 (m, 2H), 1.79-1.73 (m, 2H), 1.61-1.59 (m, 1H), 1.45-1.37 (m, 2H), 1.13-1.01 (m, 2H).

Step 2. Synthesis of 4-[[2-methyl-4-(trifluoromethyl)pyrimidin-5-yl]methyl]cyclohexanecarboxylic acid. This Compound of formula (IIa) in Scheme 1, is performed in a similar way as in example 1.1 here-above, but starting methyl 4-[[2-methyl-4-(trifluoromethyl)pyrimidin-5-yl]methyl]cyclohexanecarboxylate (423 mg, 55% purity) to give the desired intermediate (216 mg, 97%) as a white solid.

LCMS: RT 0.575 min, m/z=303.2 [M+H]⁺ (Method C).

1H NMR (400 MHz, CDCl3) δ=8.66 (s, 1H), 2.67 (d, J=7.2 Hz, 2H), 2.34-2.28 (m, 1H), 2.11 (s, 3H), 2.07-2.02 (m, 2H), 1.81-1.75 (m, 2H), 1.65-1.55 (m, 1H), 1.47-1.38 (m, 2H), 1.15-1.04 (m, 2H).

Step 3. [(3S)-3-(5-fluoro-6-methylpyridin-3-yl)-1,2-oxazolidin-2-yl]-[4-[[2-methyl-4-(trifluoromethyl)pyrimidin-5-yl]methyl]cyclohexyl]methanone (Compound (399))

This compound was synthesized in a similar manner step 5c of example 1.4 starting with 4-[[2-methyl-4-(trifluoromethyl)pyrimidin-5-yl]methyl]cyclohexanecarboxylic acid (119 mg, 0.39 mmol) and (3S)-3-(5-Fluoro-6-methyl-3-pyridyl)isoxazolidine (158 mg, 58% purity) to give Compound 399 (58.14 mg, 98%) as a white solid.

Chiral SFC. RT 1.730 min (Method M)

### Example 23: Synthesis of (358), (375) and (376)

Synthesis of [4-[(3,5-dimethyl-[1,2,4]triazolo[4,3-a]pyridin-6-yl)methyl]cyclohexyl]-[(3S)-3-pyrazin-2-yl-1,2-oxazolidin-2-yl]methanone (Compound (358)

### Step 1: Synthesis of (5-bromo-6-methyl-2-pyridyl)hydrazine

To a solution of 3-bromo-6-chloro-2-methyl pyridine (3 g, 14.53 mmol, 1 eq) in n-PrOH (25 mL) was added NH2NH2·H2O (8.56 g, 145.30 mmol, 8.31 mL, 85% purity, 10 eq). The mixture was stirred at 130° C. for 24 hr and then it was concentrated under reduced pressure to remove most of solvent. The precipitate was filtered and dried to give (5-bromo-6-methyl-2-pyridyl)hydrazine (5.5 g, crude) as a brown solid.

LCMS: RT 0.0213, m/z 204.0 [M+1]⁺

1H NMR: (DMSO-d6, 400 MHz): δ ppm 7.71-7.33 (m, 2H), 6.52 (d, J=8.8 Hz, 1H), 5.01-3.83 (m, 4H), 2.36 (s, 3H)

### Step 2: Synthesis of N′-(5-bromo-6-methyl-2-pyridyl)acetohydrazide

To a solution of (5-bromo-6-methyl-2-pyridyl)hydrazine (5.5 g, 27.22 mmol, 1 eq) in DCM (55 mL) at 0° C. was slowly added TEA (8.26 g, 81.66 mmol, 11.37 mL, 3 eq) follow by Ac2O (3.33 g, 32.66 mmol, 3.06 mL, 1.2 eq). The mixture was stirred at 25° C. for 2 hr. LCMS showed compound 2 was consumed completely. Several new peaks were shown on LCMS and 92% of desired compound was detected. The reaction mixture was concentrated under reduced pressure to give N′-(5-bromo-6-methyl-2-pyridyl)acetohydrazine (6.6 g, 21.63 mmol, 79.47% yield, 80% purity) as a white solid.

LCMS: RT 0.456, m/z 244.0 [M+1]⁺ (Method C)

### Step 3: Synthesis of 6-bromo-3,5-dimethyl-[1,2,4]triazolo[4,3-a]pyridine

A mixture of N′-(5-bromo-6-methyl-2-pyridyl)acetohydrazine (6.4 g, 26.22 mmol, 1 eq), AcOH (47.24 g, 786.60 mmol, 44.99 mL, 30 eq) in dioxane (60 mL) was degassed and purged with N₂ for 3 times, and then the mixture was stirred at 140° C. for 72 hr under N₂ atmosphere. The reaction mixture was concentrated under reduced pressure to remove dioxane and AcOH. NaHCO₃saturated aqueous solution was added to the residue until pH=8. The mixture was extracted with ethyl acetate 200 mL. The combined organic layers dried over Na2SO4, filtered and concentrated under reduced pressure, then purified by prep-HPLC (column: Phenomenex Luna C18 (250*80 mm*15 um); mobile phase: [water (0.1% FA)-ACN]; B %: 5%-35%, 20 min). 6-bromo-3,5-dimethyl-[1,2,4]triazolo[4,3-a]pyridine (2 g, 8.85 mmol, 33.74% yield) was obtained as a yellow solid

LCMS: RT 0.443; m/z 226.0 [M+1]+ (Method C)

1H NMR: (CDCL3, 400 MHz): δ 7.39 (d, J=9.7 Hz, 1H), 7.20 (d, J=9.5 Hz, 1H), 2.96 (s, 3H), 2.91 (s, 3H)

Step 4: Synthesis of methyl trans-4-[(3,5-dimethyl-[1,2,4]triazolo[4,3-a]pyridin-6-yl)methyl]cyclohexanecarboxylate—Compound of formula (IIIb) in Scheme 3, is performed in a similar way as in example 8.2—Step 1 here-above using 6-bromo-3,5-dimethyl-[1,2,4]triazolo[4,3-a]pyridine (0.5 g, 2.21 mmol) and methyl 4-(bromomethyl)cyclohexanecarboxylate (1.04 g, 4.42 mmol) to give the desired compound (210 mg, 31.4% yield) as yellow solid.

LCMS: RT 0.585 min, m/z 301.9 [M+1]+ (Method C)

1H NMR: (DMSO-d6, 400 MHz): δ ppm 7.41 (br d, J=9.3 Hz, 1H), 7.05 (br d, J=9.3 Hz, 1H), 3.56 (s, 3H), 2.95 (s, 3H), 2.76 (s, 3H), 2.47 (br s, 2H), 2.25 (br t, J=11.8 Hz, 1H), 1.87 (br d, J=11.1 Hz, 2H), 1.69 (br d, J=12.1 Hz, 2H), 1.48-1.38 (m, 1H), 1.30-1.20 (m, 2H), 1.13-1.00 (m, 2H)

Step 5: Synthesis of trans-4-[(3,5-dimethyl-[1,2,4]triazolo[4,3-a]pyridin-6-yl)methyl]cyclohexanecarboxylic acid-. Compound of formula (IIa) in Scheme 1, is performed in a similar way as in example 1.1 here-above, but starting with methyl trans-4-[(3,5-dimethyl-[1,2,4]triazolo[4,3-a]pyridin-6-yl)methyl]cyclohexanecarboxylate (210 mg, 0.52 mmol) t ogive the desired product (150 mg, 74%) as an off white solid.

LCMS: RT 0.671, m/z 288.2 [M+1]+ (Method C)

1H NMR: (DMSO-d6, 400 MHz): δ ppm 11.99 (br s, 1H), 7.41 (d, J=9.3 Hz, 1H), 7.05 (d, J=9.4 Hz, 1H), 2.95 (s, 3H), 2.76 (s, 3H), 2.47 (br s, 2H), 2.13 (tt, J=3.2, 12.0 Hz, 1H), 1.87 (br d, J=10.9 Hz, 2H), 1.69 (br d, J=10.6 Hz, 2H), 1.48-1.37 (m, 1H), 1.30-1.17 (m, 2H), 1.11-0.99 (m, 2H)

### Step 6.1: Synthesis of [4-[(3,5-dimethyl-[1,2,4]triazolo[4,3-a]pyridin-6-yl)methyl]cyclohexyl]-[(3S)-3-pyrazin-2-yl-1,2-oxazolidin-2-yl]methanone (Compound (358)

This compound was synthesized in a similar manners step 5c of example 1.4 starting with trans-4-[(3,5-dimethyl-[1,2,4]triazolo[4,3-a]pyridin-6-yl)methyl]cyclohexanecarboxylic acid (30 mg, 104 μmol) and (3S)-3-pyrazin-2-ylisoxazolidine (21.55 mg, 1.1 eq) to afford Compound (358) (26 mg, 58%) as a yellow solid

SFC: RT 1.417 min, 99.99% ee (Method F)

### Step 6.2. Synthesis of [4-(3,5-Dimethyl-[1,2,4]triazolo[4,3-a]pyridin-6-ylmethyl)-cyclohexyl]-[(S)-3-(2-methyl-thiazol-4-yl)-isoxazolidin-2-yl]-methanone (Compound 375)

This compounds was synthesized as in step 6.1 here-above, but using (3S)-3-(2-Methylthiazol-4-yl)isoxazolidine (56.2 mg, 0.33 mmol) to give Example 375 (10.0 mg, 0.023 mmol)

LCMS: RT 1.53 min, (Method A)

### Step 6.3. Synthesis of [4-(3,5-Dimethyl-[1,2,4]triazolo[4,3-a]pyridin-6-ylmethyl)-cyclohexyl]-[(S)-3-(6-methyl-pyridin-3-yl)-isoxazolidin-2-yl]-methanone (Compound 376)

This compounds was synthesized as in step 6.1 here-above, but using (3S)-3-(6-Methyl-3-pyridyl)isoxazolidine (54.2 mg, 0.33 mmol) to give Example 376 (19 mg, 0.04 mmol)

LCMS: RT 1.14 min, (Method A)

### Example 24: Synthesis of (384), (386) and (394)

Step 1. Synthesis of methyl trans-4-[(5-fluoro-2-methyl-4-pyridyl)methyl]cyclohexanecarboxylate. Compound of formula (IIIb) in Scheme 3, is performed in a similar way as in example 8.2—Step 1 here-above but using 4-Bromo-5-fluoro-2-methylpyridine (7 g, 36.8 mmol) and methyl 4-(bromomethyl)cyclohexanecarboxylate (9.12 g, 36.8 mmol) to give the desired product (3.7 g, 13.9 mmol) as a yellow oil.

LCMS: RT 0.457, m/z=266.4 [M+H]⁺ (Method C)

¹H NMR (400 MHz, CDCl₃) δ=8.25 (d, J=1.2 Hz, 1H), 6.94 (d, J=6.0 Hz, 1H), 3.66 (s, 3H), 2.56-2.46 (m, 5H), 2.25 (tt, J=12.4, 3.6 Hz, 1H), 2.04-1.93 (m, 2H), 1.83-1.72 (m, 2H), 1.67-1.51 (m, 1H), 1.40 (qd, J=13.2, 3.2 Hz, 2H), 1.04 (qd, J=13.2, 3.2 Hz, 2H).

Step 2: Synthesis of trans-4-[(5-fluoro-2-methyl-4-pyridyl)methyl]cyclohexanecarboxylic acid. The compound of formula (IIa) in Scheme 1, is performed in a similar way as in example 1.1 here-above, but starting with methyl trans-4-[(5-fluoro-2-methyl-4-pyridyl)methyl]cyclohexanecarboxylate (3.70 g, 1.39 mmol) to give the desired product (3.40 g, 95%) as a white solid.

LCMS: RT 0.474, m/z=252.2 [M+H]⁺ (Method C).

¹H NMR (400 MHz, DMSO-d₆) δ=12.01 (br s, 1H), 8.29 (d, J=1.2 Hz, 1H), 7.17 (d, J=6.0 Hz, 1H), 2.49 (d, J=6.8 Hz, 2H), 2.41 (s, 3H), 2.12 (tt, J=12.0, 3.2 Hz, 1H), 1.86 (d, J=11.2 Hz, 2H), 1.71-1.59 (m, 2H), 1.55-1.45 (m, 1H), 1.24 (qd, J=12.8, 3.0 Hz, 2H), 1.09-0.93 (m, 2H).

### Synthesis of trans-[4-[(5-fluoro-2-methylpyridin-4-yl)methyl]cyclohexyl]-[(3S)-3-(5-methylpyrazin-2-yl)-1,2-oxazolidin-2-yl]methanone (Compound (384))

This compound was synthesized in a similar manners step 5c of example 1.4 starting with trans-4-[(5-fluoro-2-methyl-4-pyridyl)methyl]cyclohexanecarboxylic acid (239 mg, 0.9 mmol) and (3S)-3-(5-methylpyrazin-2-yl)isoxazolidine (151 mg, 0.91 mmol) to give Compound 384 as a white solid (125.4 mg, 34% yield).

Chiral SFC: RT 0.943, 99.99% ee (Method X)

### Synthesis of trans-[4-[(5-fluoro-2-methylpyridin-4-yl)methyl]cyclohexyl]-[(3S)-3-(6-methylpyridin-3-yl)-1,2-oxazolidin-2-yl]methanone (Compound (386))

This compound was synthesized in a similar manners step 5c of example 1.4 starting with 4-[(5-fluoro-2-methyl-4-pyridyl)methyl]cyclohexanecarboxylic acid (300 mg, 1.19 mmol) and (3S)-3-(6-Methyl-3-pyridyl)isoxazolidine (669 mg, 29% purity) to give the Compound 386 (146 mg, 31%) as an off-white solid.

Chiral SFC: RT 1.059, 99.99% ee (Method X)

### Synthesis of trans-[4-[(5-fluoro-2-methylpyridin-4-yl)methyl]cyclohexyl]-[(3S)-3-pyrazin-2-yl-1,2-oxazolidin-2-yl]methanone (Compound (394))

This compound was synthesized in a similar manners step 5c of example 1.4 starting with 4-[(5-fluoro-2-methyl-4-pyridyl)methyl]cyclohexanecarboxylic acid (230 mg, 0.91 mmol) and (3S)-3-pyrazin-2-isoxazolidine (519 mg, 32% purity) to give Compound 394 (174 mg, 0.454 mmol) as a white solid.

Chiral SFC: RT 1.282, 99.99% ee (Method OP)

### Example 25: Synthesis of (385), (389) and (396)

Step 1. Synthesis of methyl trans-4-[(5-chloro-2-methyl-4-pyridyl)methyl]cyclohexanecarboxylate. The compound of formula (IIIb) in Scheme 3, is performed in a similar way as in example 8.2—Step 1 here-above but using 4-bromo-5-chloro-2-methyl-pyridine (5.0 g, 24.2 mmol) and methyl trans-4-(bromomethyl)cyclohexanecarboxylate (7.40 g, 31.5 mmol) to give the desired product (3.8 g, 42%) as a yellow solid.

¹H NMR (400 MHz, CDCl₃) δ=8.40 (s, 1H), 6.95 (s, 1H), 3.66 (s, 3H), 2.58 (d, J=7.2 Hz, 2H), 2.51 (s, 3H), 2.26 (ft, J=12.4, 3.6 Hz, 1H), 2.03-1.94 (m, 2H), 1.81-1.75 (m, 2H), 1.70-1.57 (m, 1H), 1.40 (qd, J=13.2, 3.2 Hz, 2H), 1.13-1.00 (m, 2H).

Step 2. Synthesis of trans-4-[(5-chloro-2-methyl-4-pyridyl)methyl]cyclohexanecarboxylic acid. This compound was synthesized in a similar manners step 5c of example 1.4 starting with methyl trans-4-[(5-chloro-2-methyl-4-pyridyl)methyl]cyclohexanecarboxylate (3.1 g, 8.58 mmol) to give the desired product (1.98 g, 86%) as a white solid.

LCMS: RT 0.333 min, m/z=268.1 [M+H]⁺ (Method C).

¹H NMR (400 MHz, DMSO-d₆) δ=8.39 (s, 1H), 7.20 (s, 1H), 2.55 (d, J=7.2 Hz, 2H), 2.42 (s, 3H), 2.14-2.08 (m, 1H), 1.89-1.83 (m, 2H), 1.66-1.51 (m, 3H), 1.27-1.17 (m, 2H), 1.09-0.98 (m, 2H).

Step 3.1. Synthesis of trans-[4-[(5-chloro-2-methylpyridin-4-yl)methyl]cyclohexyl]-[(3S)-3-(5-methylpyrazin-2-yl)-1,2-oxazolidin-2-yl]methanone (Compound (385)). This compound was synthesized in a similar manner as step 5c of example 1.4 starting with 4-[(5-chloro-2-methyl-4-pyridyl)methyl]cyclohexanecarboxylic acid (320 mg, 1.2 mmol) and (3S)-3-(5-methylpyrazin-2-yl)isoxazolidine (199 mg, 1 eq) to give Example 385 (278 mg, 56%) as a white solid.

Chiral SFC: RT 1.855 min, 99.99% ee (Method AO)

Step 3.2. Synthesis of trans-[4-[(5-chloro-2-methylpyridin-4-yl)methyl]cyclohexyl]-[(3S)-3-(6-methylpyridin-3-yl)-1,2-oxazolidin-2-yl]methanone (Compound (389)).This compound was synthesized in a similar manner as step 5c of example 1.4 starting with 4-[(5-chloro-2-methyl-4-pyridyl)methyl]cyclohexanecarboxylic acid (260 mg, 0.97 mmol) and (3S)-3-(6-Methyl-3-pyridyl)isoxazolidine (753 mg, 21% purity) to give compound 389 (201.6 mg, 50%) as a yellow gum.

Chiral SFC: RT 1.150 min, 99.99% ee (Method P)

### Step 3.3. Synthesis of trans-[4-[(5-chloro-2-methylpyridin-4-yl)methyl]cyclohexyl]-[(3S)-3-pyrazin-2-yl-1,2-oxazolidin-2-yl]methanone (Compound (396))

This compound was synthesized in a similar manner as step 5c of example 1.4 starting with trans 4-[(5-chloro-2-methyl-4-pyridyl)methyl]cyclohexanecarboxylic acid (330 mg, 1.23 mmol) and (3S)-3-pyrazin-2-isoxazolidine (448 mg, 32% purity) to give Compound 396 (261.9 mg, 69%) as an off-white solid.

Chiral SFC: RT 1.088 min, 99.99% ee (Method P)

### Example 26: Synthesis of Compounds (400), (401), (402), (403)

### STEP 1. Synthesis of cis: trans methyl 4-[(2,8-dimethyl-[1,2,4]triazolo[1,5-a]pyridin-6-yl)methyl]-1-methyl-cyclohexanecarboxylate

The compound of formula (IIIb) in Scheme 3, is performed in a similar way as in example 8.2—Step 1 here-above using 6-bromo-2,8-dimethyl-[1,2,4]triazolo[1,5-a]pyridine and methyl 4-(bromomethyl)-1-methyl-cyclohexanecarboxylate (cis:trans mixture) to give cis:trans methyl 4-[(2,8-dimethyl-[1,2,4]triazolo[1,5-a]pyridin-6-yl)methyl]-1-methyl-cyclohexanecarboxylate (1.3 g, 60% purity).

### STEP 2. Synthesis of cis: trans 4-[(2,8-dimethyl-[1,2,4]triazolo[1,5-a]pyridin-6-yl)methyl]-1-methyl-cyclohexanecarboxylic Acid

The compound of formula (IIa) in Scheme 1, is performed in a similar way as in example 1.1 but starting with cis:trans methyl 4-[(2,8-dimethyl-[1,2,4]triazolo[1,5-a]pyridin-6-yl)methyl]-1-methyl-cyclohexanecarboxylate (1.3 g, 60% purity) to give cis: trans 4-[(2,8-dimethyl-[1,2,4]triazolo[1,5-a]pyridin-6-yl)methyl]-1-methyl-cyclohexanecarboxylic acid (840 mg, 93% purity). The two isomers were purified by SFC (column: DAICEL CHIRALPAK IK (250 mm*30 mm, 10 um); mobile phase: CO2-ACN/EtOH (1‰NH3·H2O); B %: 20%, isocratic elution mode) to give trans-4-[(2,8-dimethyl-[1,2,4]triazolo[1,5-a]pyridin-6-yl)methyl]-1-methyl-cyclohexanecarboxylic acid (760 mg) and cis-4-[(2,8-dimethyl-[1,2,4]triazolo[1,5-a]pyridin-6-yl)methyl]-1-methyl-cyclohexanecarboxylic acid (400 mg) Trans-4-[(2,8-dimethyl-[1,2,4]triazolo[1,5-a]pyridin-6-yl)methyl]-1-methyl-cyclohexanecarboxylic acid (760 mg) was obtained as a white solid

LCMS: RT 0.475 min, m/z=302.2 [M+H]+ (Method Q)

Chiral SFC: RT 1.117 min, 99.99% ee (Method AF)

1H NMR (400 MHz, DMSO-d6) δ=12.06 (br s, 1H), 8.45 (s, 1H), 7.26 (s, 1H), 2.48-2.44 (m, 5H), 2.43 (s, 3H), 2.09-1.97 (m, 2H), 1.58-1.42 (m, 3H), 1.09-0.95 (m, 7H).

Cis-4-[(2,8-dimethyl-[1,2,4]triazolo[1,5-a]pyridin-6-yl)methyl]-1-methyl-cyclohexanecarboxylic acid (400 mg) was obtained as a white solid.

1H NMR (400 MHz, DMSO-d6) δ=11.40 (br s, 1H), 8.45 (s, 1H), 7.28 (s, 1H), 2.54 (d, J=7.2 Hz, 2H), 2.46 (s, 3H), 2.43 (s, 3H), 1.64-1.42 (m, 7H), 1.24-1.12 (m, 2H), 1.11 (s, 3H).

LCMS: RT 0.472 min, m/z=302.2 [M+H]+ (Method Q)

Chiral SFC: RT 1.382 min, % ee (Method AF)

### STEP 3.1 Synthesis of trans-[4-[(2,8-dimethyl-[1,2,4]triazolo[1,5-a]pyridin-6-yl)methyl]-1-methylcyclohexyl]-[(3S)-(6-methylpyridin-3-yl)-1,2-oxazolidin-2-yl]methanone (Compound 400)

This compound was synthesized in a similar manner as step 5c of example 1.4 starting with trans-4-[(2,8-dimethyl-[1,2,4]triazolo[1,5-a]pyridin-6-yl)methyl]-1-methyl-cyclohexanecarboxylic acid (70 mg, 0.23 mmol) and (3S)-3-(6-Methyl-3-pyridyl)isoxazolidine (58 mg, 65% purity) to afford trans-[4-[(2,8-dimethyl-[1,2,4]triazolo[1,5-a]pyridin-6-yl)methyl]-1-methylcyclohexyl]-[(3S)-(6-methylpyridin-3-yl)-1,2-oxazolidin-2-yl]methanone (Compound 400) (44.38 mg, 99%) as a pink solid.

Chiral SFC: RT 1.220 min, 99.99% de (Method X)

### STEP 3.2. Synthesis of trans-[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone (Compound (401))

This compound was synthesized in a similar manner as step 5c of example 1.4 starting with trans-4-[(2,8-dimethyl-[1,2,4]triazolo[1,5-a]pyridin-6-yl)methyl]-1-methyl-cyclohexanecarboxylic acid (70 mg, 0.23 mmol) and (3S)-3-(6-Methyl-3-pyridyl)isoxazolidine (70 mg, 65% purity) to afford trans-[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone (Compound (401)) (49.4 mg, 99%) as a pink solid.

Chiral SFC: RT 1.015 min, 99.99% de (Method X)

### STEP 3.3. Synthesis of cis-[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone (Compounds (402))

This compound was synthesized in a similar manner as step 5c of example 1.4 starting with cis-4-[(2,8-dimethyl-[1,2,4]triazolo[1,5-a]pyridin-6-yl)methyl]-1-methyl-cyclohexanecarboxylic acid (70 mg, 0.23 mmol) and (3S)-3-(5-Fluoro-6-methyl-3-pyridyl)isoxazolidine (63 mg, 69% purity) to afford cis-[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone (Compounds (402)) (53 mg) as a pink solid.

Chiral SFC: RT 0.984 min, 99.99% de (Method X)

### STEP 3.4. Synthesis of trans-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone (Compound (403))

This compound was synthesized in a similar manner as step 5c of example 1.4 starting with trans-4-[(2,8-dimethyl-[1,2,4]triazolo[1,5-a]pyridin-6-yl)methyl]-1-methyl-cyclohexanecarboxylic acid (70 mg, 0.23 mmol) and (3S)-3-(5-Fluoro-6-methyl-3-pyridyl)isoxazolidine (73 mg, 69% purity) to afford trans[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone (Compound (403) (63 mg, 59%) as a pink solid.

Chiral SFC: RT 1.103 min, 99.99% de (Method X)

### Example 27: Synthesis of Compounds (405), (406), (407) and (408)

### STEP 1. Synthesis of Cis:Trans tert-butyl 4-[(2,8-dimethyl-[1,2,4]triazolo[1,5-a]pyridin-6-yl)methyl]-4-methyl-cyclohexanecarboxylate

The compound of formula (IIIb) in Scheme 3, is performed in a similar way as in example 8.2—Step 1 here-above using tert-butyl 4-(hydroxymethyl)-4-methyl-cyclohexanecarboxylate (cis:trans mixture) (3.00 g, 0.0161 mol, 1.00 eq.), NHC (10.2 g, 0.0258 mol, 1.60 eq., CAS: 1207294-92-5), and 6-bromo-2,8-dimethyl-[1,2,4]triazolo[1,5-a]pyridine (5.46 g, 0.0242 mol, 1.50 eq.), to afford cis:trans tert-butyl 4-[(2,8-dimethyl-[1,2,4]triazolo[1,5-a]pyridin-6-yl)methyl]-4-methyl-cyclohexanecarboxylate (4.70 g, 30.0% purity, 0.00447 mol, 27.8% yield) as a yellow solid.

1H NMR (400 MHz, CDCl3) δ=8.09 (s, 1H), 7.04 (s, 1H), 2.59-2.55 (m, 7H), 2.48 (s, 1H), 2.27-2.17 (m, 0.4 H), 2.11-2.01 (m, 0.6 H), 1.76-1.71 (m, 2H), 1.63-1.49 (m, 3H), 1.45 (s, 4H), 1.42 (s, 5H), 1.33-1.15 (m, 3H), 0.90 (s, 1.5 H), 0.83 (s, 1.5 H).

### STEP 2. Synthesis of Cis and Trans 4-[(2,8-dimethyl-[1,2,4]triazolo[1,5-a]pyridin-6-yl)methyl]-4-methyl-cyclohexanecarboxylic Acid

To a solution of cis:trans tert-butyl 4-[(2,8-dimethyl-[1,2,4]triazolo[1,5-a]pyridin-6-yl)methyl]-4-methyl-cyclohexanecarboxylate (4.70 g, 30.0% purity, 0.00394 mol, 1.00 eq.) in DCM (15 mL) was added TFA (15 mL). The mixture was stirred at 25° C. for 2 h and then it was concentrated under reduced pressure. The residue was dissolved into water (10 mL), adjusted to pH 9-10 with NaOH (1 M in water) and then washed with EtOAc (2×30 mL). Afterwards, the left aqueous layer was adjusted to pH 4-5 with citric acid (sat. aq.), and then extracted with EtOAc (3×60 mL). The combined organic layers were dried over anhydrous Na₂SO₄ solid, filtered and concentrated under reduced pressure to afford cis:trans 4-[(2,8-dimethyl-[1,2,4]triazolo[1,5-a]pyridin-6-yl)methyl]-4-methyl-cyclohexanecarboxylic acid (1.80 g, ˜66.0% purity, 0.00394 mol, 99.9% yield) as a white solid. This was further purified by SFC (column: DAICEL CHIRALPAKAD (250 mm*50 mm, 10 um); mobile phase: CO₂-i-PrOH (1‰ NH₃·H₂O); B %: 40%, isocratic elution mode to afford cis-4-[(2,8-dimethyl-[1,2,4]triazolo[1,5-a]pyridin-6-yl)methyl]-4-methyl-cyclohexanecarboxylic acid (490 mg) and trans-4-[(2,8-dimethyl-[1,2,4]triazolo[1,5-a]pyridin-6-yl)methyl]-4-methyl-cyclohexanecarboxylic acid cis-4-[(2,8-dimethyl-[1,2,4]triazolo[1,5-a]pyridin-6-yl)methyl]-4-methyl-cyclohexanecarboxylic acid (490 mg) was obtained as a white solid.

LCMS: RT 0.428 min m/z=302.3 [M+H]+ (Method C)

1H NMR (400 MHz, DMSO-d6) δ=12.03 (br s, 1H), 8.38 (s, 1H), 7.19 (s, 1H), 2.58 (s, 2H), 2.47 (s, 3H), 2.43 (s, 3H), 2.29-2.19 (m, 1H), 1.82-1.65 (m, 4H), 1.49-1.38 (m, 2H), 1.22-1.11 (m, 2H), 0.79 (s, 3H).

Chiral SFC: RT 1.764 min, 97% de (Method AG)

trans-4-[(2,8-dimethyl-[1,2,4]triazolo[1,5-a]pyridin-6-yl)methyl]-4-methyl-cyclohexanecarboxylic acid (680 mg, was obtained as a white solid.

LCMS: RT 0.423 min m/z=302.3 [M+H]+ (Method C)

1H NMR (400 MHz, DMSO-d6) δ=11.98 (br s, 1H), 8.41 (s, 1H), 7.22 (s, 1H), 2.49 (s, 2H), 2.47 (s, 3H), 2.44 (s, 3H), 2.07 (t, J=11.6, 3.6 Hz, 1H), 1.76-1.64 (m, 2H), 1.54-1.40 (m, 2H), 1.39-1.22 (m, 4H), 0.84 (s, 3H).

Chiral SFC: RT 1.462 min, 97% de (Method AG)

### STEP 3.1. Synthesis of cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone (Compound 405)

This compound was synthesized in a similar manner as step 5c of example 1.4 starting with cis-4-[(2,8-dimethyl-[1,2,4]triazolo[1,5-a]pyridin-6-yl)methyl]-4-methyl-cyclohexanecarboxylic acid (70 mg, 0.23 mmol) and (3S)-3-(5-fluoro-6-methyl-3-pyridyl)isoxazolidine (73 mg, 79% purity) to cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone (Compound 405) (66 mg, 61%) as a white solid.

Chiral SFC: RT 0.953 min, 99.99% de (Method X)

### STEP 3.2. Synthesis of cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone (Compound (406))

This compound was synthesized in a similar manner as step 5c of example 1.4 starting with cis-4-[(2,8-dimethyl-[1,2,4]triazolo[1,5-a]pyridin-6-yl)methyl]-4-methyl-cyclohexanecarboxylic acid (70 mg, 0.23 mmol) and (3S)-3-(5-fluoro-6-methyl-3-pyridyl)isoxazolidine (70 mg, 59% purity) to cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone (Compound 406) (49 mg, 45%) as a white solid.

Chiral SFC: RT 1.147 min, 99.99% de (Method X)

### STEP 3.3 Synthesis of cis-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone (Compound (407))

This compound was synthesized in a similar manner as step 5c of example 1.4 starting with cis-4-[(2,8-dimethyl-[1,2,4]triazolo[1,5-a]pyridin-6-yl)methyl]-4-methyl-cyclohexanecarboxylic acid (70 mg, 0.23 mmol) and (3S)-3-(6-methyl-3-pyridyl)isoxazolidine (70 mg, 59% purity) to cis-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone (Compound (407)) (44 mg, 42%) as a white solid.

Chiral SFC: RT 1.105 min, 99.99% de (Method X)

### STEP 3.4. Synthesis of trans-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone (Compound (408))

This compound was synthesized in a similar manner as step 5c of example 1.4 starting with trans-4-[(2,8-dimethyl-[1,2,4]triazolo[1,5-a]pyridin-6-yl)methyl]-4-methyl-cyclohexanecarboxylic acid (70 mg, 0.23 mmol) and (3S)-3-(6-methyl-3-pyridyl)isoxazolidine (70 mg, 59% purity) to afford trans-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone (Compound (408)) (42 mg, 40%) as a white solid.

Chiral SFC: RT 1.397 min, 99.99% de (Method X)

### Example 28—Synthesis of (410)

### STEP 1. Synthesis of Methyl trans-4-[(1,3-dimethylpyrazolo[4,3-b]pyridin-6-yl)methyl]cyclohexanecarboxylate

The compound of formula (IIIb) in Scheme 3, is performed in a similar way as in example 8.2—Step 1 here-above using methyl trans-4-(bromomethyl)cyclohexanecarboxylate (2.7 g, 11.5 mmol) and 6-bromo-1,3-dimethyl-pyrazolo[4,3-b]pyridine (2.00 g, 8.8 mmol) to afford the above intermediate as a yellow oil (1.06 g, 71% purity, 28%).

LCMS: RT 0.531 min m/z=288.1 [M+H]+ (Method Q)

### STEP 2: Synthesis of trans-4-[(1,3-dimethylpyrazolo[4,3-b]pyridin-6-yl)methyl]cyclohexanecarboxylic Acid

The compound of formula (IIa) in Scheme 1, is performed in a similar way as in example 1.1 but starting with methyl trans-4-[(1,3-dimethylpyrazolo[4,3-b]pyridin-6-yl)methyl]cyclohexanecarboxylate (1.06 g, 71% purity) to afford the above intermediate as a yellow solid (496 mg, 69%).

LCMS: RT 0.739 min m/z=288.1 [M+H]+ (Method C)

¹H NMR (400 MHz, DMSO-d₆) δ=11.99 (br s, 1H), 8.29 (d, J=1.6 Hz, 1H), 7.81 (d, J=1.6 Hz, 1H), 3.95 (s, 3H), 2.70-2.60 (m, 2H), 2.48 (s, 3H), 2.19-2.03 (m, 1H), 1.86 (d, J=10.8 Hz, 2H), 1.71-1.61 (m, 2H), 1.61-1.50 (m, 1H), 1.34-1.18 (m, 2H), 1.09-0.95 (m, 2H).

### STEP 3. Synthesis of [4-[(1,3-dimethylpyrazolo[4,3-b]pyridin-6-yl)methyl]cyclohexyl]-[(3S)-3-(5-methylpyrazin-2-yl)-1,2-oxazolidin-2-yl]methanone (Compound (410))

This compound was synthesized in a similar manner as step 5c of example 1.4 starting with trans-4-[(1,3-dimethylpyrazolo[4,3-b]pyridin-6-yl)methyl]cyclohexanecarboxylic acid (160 mg, 0.5 mmol) and (3S)-3-(5-methylpyrazin-2-yl)isoxazolidine (137 mg, 67% purity) to afford [4-[(1,3-dimethylpyrazolo[4,3-b]pyridin-6-yl)methyl]cyclohexyl]-[(3S)-3-(5-methylpyrazin-2-yl)-1,2-oxazolidin-2-yl]methanone (Compound (410)) (159 mg) as a white solid.

Chiral SFC: RT 1.346 min, 99.99% ee (Method X)

### Example 29: Biological Activity

### Evaluation of Receptor-Interacting Protein Kinase 1 Inhibition.

The catalytic activity of RIPK1 was measured by monitoring the conversion of Adenosine triphosphate (ATP) to Adenosine diphosphate (ADP) due to autophosphorylation using an ADP-Glo kinase kit (Promega, catalog no. V9104).

In detail, 2 μl recombinantly produced hRIPK1 (aa 1-375) fusion protein (end concentration 3.6 μg/ml) and 2 μl compound (end concentration 33300-1.69 nM; DMSO end concentration 1%) were incubated for 30 minutes at room temperature and then 2 μl ATP (ADP Glo kit, end concentration 50 μM) were added. After another 240 minutes incubation at room temperature, 5 μl Promega ADP-Glo reagent I was added to quench the reaction and deplete unconsumed ATP. After an incubation period of 30 minutes, 10 μl Promega ADP-Glo detection reagent II was added resulting in conversion of ADP to ATP, which generates a light-reaction between luciferase and luciferin. Luminescence was quantified after 30 minutes with a Pherastar FS (BMG LABTECH, Ortenberg).

For the dose response experiments an IC₅₀ value with 95% confidence interval was calculated using the 4-parameter logistic model according to Ratkowsky and Reedy with constraints for lower and upper asymptotes at 0% and 100%. The adjustment was obtained by nonlinear regression using the Levenberg Marquardt algorithm.

### Cellular Assay in U937 Cells to Measure the Activity of RIPK1-Inhibitors on Cell Death (Necroptosis).

Upon TNF-Receptor I ligation, Ser/Thr kinase RIPK1 is recruited to a transient receptor complex 1. Upon modification of RIPK1 which promotes activation of RIPK1, complex IIb can form, that involves recruitment of RIPK3 and MLKL (mixed lineage-kinase domain-like protein) which then translocates from the cytosol to the plasma membrane to execute cell death (Cai, Z. et al, Nat. Cell Biol. (2014) 16:55-65).

Cell death was quantified in 96 well plates by determination of the amount of live cell using a CellTiter 96 AQueous reagent (Promega), a calorimetric method to measure the number of live cells by reducing tetrazolium compound [3-(4,5-dimethylthiazol-2-yl)-5-(3-carboxymethoxyphenyl)-2-(4-sulfophenyl)-2H-tetrazolium, inner salt; MTS] into formazan. Absorbance of formazan was read at 490 nm. The inhibitory activity of the test compound was quantified in a concentration response curve (CRC) experiment.

Compounds were obtained as 10 mM stock solutions and were diluted 1 to 10 volumes with DMSO to yield a 1 mM solution. From this solution 2 μl were diluted with 998 μl growth medium. 100 μl of 2 μM compound solution was further diluted sequentially with a dilution factor of 2.5 by adding 150 μl growth medium. A total of 10 concentrations were tested ranging from 10 μM to 0.26 nM or from 1 μM to 0.07 nM.

U937 cells were cultured in RPMI1640 Glutamax and 10% heat inactivated FBS. 50 μl cell suspension containing 1×106 cells/ml supplemented with 50 μM zVAD.fmk (Benzyloxycarbonyl-Val-Ala-Asp (OMe) fluoromethylketone) and 100 ng/ml recombinant human TNF□ were dispensed in each well of a 96-well plate. 50 μl of compound dilutions (described in Cellular assay in U937 cells) were added and the cell suspension incubated overnight (18 to 24 hrs) at 37° C., 5% CO₂ in a humidified atmosphere (95% rH). High (no compound) and low control (no TNF□, zVAD.fmk) were tested with 7 replicates; all compound concentrations were tested in duplicates on each experimental plate.

CellTiter 96 Aqueous reagent was mixed (100 μl PMS (phenazine methosulfate) solution/2 ml MTS (3-(4,5-dimethyldiazol-2-yl)-5-(3-carboxymethoxyphenyl)-2-(4-sulfophenyl)-2H-tetrazolium, inner salt) solution) and 20 μl were added per well. After 4 hrs incubation at 37° C. (5% CO₂ 95% rH) optical density was measured at 490 nm on a microplate reader (Tecan Infinite M1000).

The % inhibition is expressed as percentage of the maximal inhibition value obtained in the absence of TNFα/zVAD.fmc. For each dose response experiment an IC₅₀ value with 95% confidence interval was calculated using the 4-parameter logistic model according to Ratkowsky and Reedy without constraints using an internal application (Biost@t-Speed LTS V2.3).

Results of biological activity are shown in Table 2 (ADP-Glo IC₅₀ (μM) and U937 IC₅₀ (μM)).

TABLE 2
activity data of the examples

| | ADP Glo | U937 cell |
|---|---|---|
| Cpd | assay IC₅₀ | assay IC₅₀ |
| No | [nM] | [nM] |
| 1 | 21 | 29 |
| 2 | 33 | 9 |
| 3 | 22 | 18 |
| 4 | 116 | 721 |
| 5 | 45 | 244 |
| 6 | 154 | 377 |
| 7 | 15 | 9 |
| 8 | 19 | 6 |
| 9 | 113 | 174 |
| 10 | 43 | 8 |
| 11 | 59 | 38 |
| 12 | 33 | 3 |
| 13 | 33 | 5 |
| 14 | 50 | 3 |
| 15 | 46 | 11 |
| 16 | 39 | 6 |
| 17 | 21 | 2 |
| 18 | 19 | 16 |
| 19 | 28 | 5 |
| 20 | 20 | 5 |
| 21 | 25 | 5 |
| 22 | 20 | 2 |
| 23 | 21 | 3 |
| 24 | 109 | 471 |
| 25 | 68 | 154 |
| 26 | 20 | 2 |
| 27 | 6 | 2 |
| 28 | 19 | 3 |
| 29 | 12 | 1 |
| 30 | 39 | 68 |
| 31 | 24 | 19 |
| 32 | 19 | 3 |
| 33 | 19 | 2 |
| 34 | 37 | 69 |
| 35 | 41 | 48 |
| 36 | 23 | 20 |
| 37 | 17 | 5 |
| 38 | 32 | 30 |
| 39 | 28 | 15 |
| 40 | 41 | 59 |
| 41 | 30 | 91 |
| 42 | 39 | 119 |
| 43 | 18 | 1 |
| 44 | 17 | 2 |
| 45 | 18 | 6 |
| 46 | 13 | 6 |
| 47 | 18 | 3 |
| 48 | 25 | 15 |
| 49 | 21 | 8 |
| 50 | 27 | 29 |
| 51 | 34 | 17 |
| 52 | 15 | 2 |
| 53 | 17 | 4 |
| 54 | 21 | 6 |
| 55 | 21 | 3 |
| 56 | 42 | 109 |
| 57 | 27 | 41 |
| 58 | 26 | 32 |
| 59 | 30 | 166 |
| 60 | 14 | 5 |
| 61 | 23 | 11 |
| 62 | 20 | 22 |
| 63 | 27 | 55 |
| 64 | 20 | 47 |
| 65 | 51 | 164 |
| 66 | 18 | 2 |
| 67 | 15 | 2 |
| 68 | 25 | 2 |
| 69 | 22 | 5 |
| 70 | 44 | 403 |
| 71 | 37 | 93 |
| 72 | 30 | 10 |
| 73 | 24 | 9 |
| 74 | 28 | 106 |
| 75 | 27 | 36 |
| 76 | 17 | 8 |
| 77 | 14 | 3 |
| 78 | 40 | 72 |
| 79 | 22 | 13 |
| 80 | 60 | 745 |
| 81 | 33 | 166 |
| 82 | 67 | 136 |
| 83 | 32 | 22 |
| 84 | 13 | 4 |
| 85 | 21 | 3 |
| 86 | 14 | 1 |
| 87 | 18 | 3 |
| 88 | 52 | 157 |
| 89 | 34 | 38 |
| 90 | 75 | 147 |
| 91 | 39 | 67 |
| 92 | 75 | 132 |
| 93 | 20 | 2 |
| 94 | 19 | 1 |
| 95 | 22 | 4 |
| 96 | 16 | 2 |
| 97 | 78 | 214 |
| 98 | 32 | 84 |
| 99 | 37 | 33 |
| 100 | 17 | 4 |
| 101 | 18 | 4 |
| 102 | 23 | 37 |
| 103 | 23 | 22 |
| 104 | 18 | 5 |
| 105 | 15 | 4 |
| 106 | 46 | 54 |
| 107 | 39 | 108 |
| 108 | 27 | 18 |
| 109 | 19 | 1 |
| 110 | 22 | 11 |
| 111 | 27 | 10 |
| 112 | 23 | 7 |
| 113 | 24 | 3 |
| 114 | 34 | 174 |
| 115 | 31 | 138 |
| 116 | 57 | 491 |
| 117 | 35 | 54 |
| 118 | 23 | 30 |
| 119 | 27 | 54 |
| 120 | 41 | 6 |
| 121 | 30 | 25 |
| 122 | 125 | 797 |
| 123 | 46 | 12 |
| 124 | 26 | 68 |
| 125 | 9 | 16 |
| 126 | 37 | 29 |
| 127 | 29 | 15 |
| 128 | 22 | 4 |
| 129 | 29 | 7 |
| 130 | 11 | 2 |
| 131 | 15 | 5 |
| 132 | 13 | 4 |
| 133 | 8 | 8 |
| 134 | 27 | 25 |
| 135 | 25 | 65 |
| 136 | 26 | 26 |
| 137 | 30 | 46 |
| 138 | 18 | 25 |
| 139 | 10 | 2 |
| 140 | 40 | 173 |
| 141 | 13 | 17 |
| 142 | 38 | 4 |
| 143 | 76 | 44 |
| 144 | 22 | 3 |
| 145 | 29 | 16 |
| 146 | 28 | 26 |
| 147 | 27 | 40 |
| 148 | 18 | 4 |
| 149 | 9 | 8 |
| 150 | 21 | 25 |
| 151 | 46 | 214 |
| 152 | 7 | 3 |
| 153 | 6 | 3 |
| 154 | 23 | 33 |
| 155 | 15 | 19 |
| 156 | 16 | 6 |
| 157 | 33 | 396 |
| 158 | 9 | 33 |
| 159 | 7 | 48 |
| 160 | 6 | 11 |
| 161 | 4 | 1 |
| 162 | 4 | 15 |
| 163 | 5 | 10 |
| 164 | 5 | 7 |
| 165 | 5 | 7 |
| 166 | 8 | 50 |
| 167 | 104 | 100 |
| 168 | 829 | 653 |
| 169 | 19 | 44 |
| 170 | 21 | 15 |
| 171 | 19 | 6 |
| 172 | 16 | 9 |
| 173 | 18 | 17 |
| 174 | 19 | 4 |
| 175 | 14 | 7 |
| 176 | 13 | 15 |
| 177 | 11 | 5 |
| 178 | 38 | 6 |
| 179 | 32 | 5 |
| 180 | 32 | 11 |
| 181 | 26 | 2 |
| 182 | 22 | 1 |
| 183 | 45 | 23 |
| 184 | 26 | 19 |
| 185 | 32 | 34 |
| 186 | 23 | 39 |
| 187 | 51 | 121 |
| 188 | 37 | 124 |
| 189 | 138 | 623 |
| 190 | 13 | 4 |
| 191 | 10 | 9 |
| 192 | 5 | 3 |
| 193 | 10 | 14 |
| 194 | 21 | 10 |
| 195 | 12 | 6 |
| 196 | 75 | 379 |
| 197 | 24 | 4 |
| 198 | 17 | 7 |
| 199 | 18 | 51 |
| 200 | 29 | 4 |
| 201 | 62 | 190 |
| 202 | 19 | 39 |
| 203 | 125 | 432 |
| 204 | 21 | 2 |
| 205 | 31 | 112 |
| 206 | 16 | 9 |
| 207 | 23 | 53 |
| 208 | 24 | 12 |
| 209 | 22 | 54 |
| 210 | 17 | 25 |
| 211 | 18 | 20 |
| 212 | 23 | 10 |
| 213 | 19 | 16 |
| 214 | 20 | 4 |
| 215 | 20 | 7 |
| 216 | 15 | 10 |
| 217 | 41 | 12 |
| 218 | 20 | 13 |
| 219 | 17 | 3 |
| 220 | 26 | 12 |
| 221 | 22 | 19 |
| 222 | 37 | 48 |
| 223 | 19 | 5 |
| 224 | 35 | 17 |
| 225 | 19 | 7 |
| 226 | 44 | 40 |
| 227 | 23 | 5 |
| 228 | 65 | 191 |
| 229 | 25 | 78 |
| 230 | 131 | 376 |
| 231 | 23 | 35 |
| 232 | 69 | 723 |
| 233 | 114 | 384 |
| 234 | 49 | 126 |
| 235 | 35 | 152 |
| 236 | 20 | 4 |
| 237 | 23 | 66 |
| 238 | 41 | 257 |
| 239 | 27 | 5 |
| 240 | 27 | 52 |
| 241 | 148 | 28 |
| 242 | 21 | 16 |
| 243 | 13 | 3 |
| 244 | 20 | 0 |
| 245 | 17 | 16 |
| 246 | 150 | 918 |
| 247 | 22 | 136 |
| 248 | 73 | 666 |
| 249 | 18 | 5 |
| 250 | 16 | 17 |
| 251 | 8 | 17 |
| 252 | 17 | 54 |
| 253 | 18 | 175 |
| 254 | 20 | 96 |
| 255 | 25 | 521 |
| 256 | 15 | 1 |
| 257 | 10 | 2 |
| 258 | 71 | 862 |
| 259 | 8 | 2 |
| 260 | 32 | 25 |
| 261 | 23 | 17 |
| 262 | 34 | 67 |
| 263 | 41 | 59 |
| 264 | 14 | 16 |
| 265 | 8 | 1 |
| 266 | 13 | 1 |
| 267 | 12 | 2 |
| 268 | 12 | 5 |
| 269 | 13 | 28 |
| 270 | 20 | 50 |
| 271 | 97 | 605 |
| 272 | 29 | 206 |
| 273 | 15 | 9 |
| 274 | 9 | 40 |
| 275 | 8 | 9 |
| 276 | 20 | 9 |
| 277 | 19 | 4 |
| 278 | 16 | 3 |
| 279 | 7 | 9 |
| 280 | 13 | 7 |
| 281 | 19 | 5 |
| 282 | 18 | 10 |
| 283 | 16 | 13 |
| 284 | 17 | 34 |
| 285 | 11 | 10 |
| 286 | 1 | 1 |
| 287 | 2 | 1 |
| 288 | 17 | 4 |
| 289 | 36 | 733 |
| 290 | 11 | 4 |
| 291 | 12 | 1 |
| 292 | 14 | 4 |
| 293 | 14 | 3 |
| 294 | 4 | 1 |
| 295 | 6 | 1 |
| 296 | 5 | 2 |
| 297 | 14 | 7 |
| 298 | 20 | 6 |
| 299 | 14 | 19 |
| 300 | 19 | 13 |
| 301 | 33 | 34 |
| 302 | 31 | 37 |
| 303 | 7 | 16 |
| 304 | 23 | 33 |
| 305 | 29 | 43 |
| 306 | 17 | 25 |
| 307 | 15 | 2 |
| 308 | 21 | 47 |
| 309 | 15 | 50 |
| 310 | 12 | 22 |
| 311 | 11 | 16 |
| 312 | 38 | 20 |
| 313 | 42 | 40 |
| 314 | 35 | na |
| 315 | 10 | 20 |
| 316 | 31 | 27 |
| 317 | 7 | 2 |
| 318 | 22 | 27 |
| 319 | 14 | 33 |
| 320 | 37 | 28 |
| 321 | 18 | 24 |
| 322 | 37 | 27 |
| 323 | 10 | 6 |
| 324 | 34 | 35 |
| 325 | 125 | 108 |
| 326 | 51 | 59 |
| 327 | 48 | 48 |
| 328 | 47 | 82 |
| 329 | 44 | 82 |
| 330 | 20 | 18 |
| 331 | 15 | 20 |
| 332 | 12 | 3 |
| 333 | 30 | 55 |
| 334 | 20 | 35 |
| 335 | 27 | 11 |
| 336 | 17 | 10 |
| 337 | 26 | 31 |
| 338 | 29 | 45 |
| 339 | 25 | 16 |
| 340 | 25 | 77 |
| 341 | 81 | 114 |
| 342 | 26 | 8 |
| 343 | 43 | 21 |
| 344 | 243 | 173 |
| 345 | 26 | 8 |
| 346 | 83 | 70 |
| 347 | 46 | 59 |
| 348 | 37 | 105 |
| 349 | 42 | 108 |
| 350 | 63 | 34 |
| 351 | 210 | 149 |
| 352 | 30 | 58 |
| 353 | 87 | 112 |
| 354 | 33 | 27 |
| 356 | 26 | 5 |
| 357 | 138 | 531 |
| 358 | 31 | 481 |
| 359 | 104 | 451 |
| 360 | 20 | 7 |
| 361 | 57 | 35 |
| 362 | 59 | 52 |
| 363 | 180 | 222 |
| 364 | 234 | 491 |
| 365 | 97 | 71 |
| 366 | 40 | 11 |
| 367 | 94 | — |
| 368 | 95 | 123 |
| 369 | 26 | 24 |
| 370 | 34 | 21 |
| 371 | 65 | 147 |
| 372 | 53 | 103 |
| 373 | 110 | 78 |
| 374 | 36 | 55 |
| 375 | 63 | 10 |
| 376 | 75 | 90 |
| 377 | 35 | 36 |
| 378 | 59 | 95 |
| 379 | 50 | 191 |
| 380 | 70 | 129 |
| 381 | 31 | 8 |
| 382 | 26 | 8 |
| 383 | 81 | 47 |
| 384 | 28 | 7 |
| 385 | 24 | 3 |
| 386 | 25 | 13 |
| 387 | 995 | 1881 |
| 388 | 97 | 176 |
| 389 | 20 | 13 |
| 390 | 63 | 58 |
| 391 | 63 | 64 |
| 392 | 32 | 18 |
| 393 | 51 | 33 |
| 394 | 14 | 5 |
| 395 | 44 | 45 |
| 396 | 14 | 4 |
| 397 | 61 | 163 |
| 398 | 64 | 182 |
| 399 | 85 | 274 |
| 400 | 19 | 9 |
| 402 | 2000 | 1128 |
| 403 | 15 | 2 |
| 404 | 15 | 22 |
| 409 | 46 | 66 |
| 410 | 80 | — |
| 411 | 48 | 36 |

All the compounds according to the disclosure are potent RIPK1 inhibitors as they exhibit a reducing catalytic activity of RIPK1, highlighted by ADP Glo assay, with IC₅₀ lower than 900 nM. Most of the compounds even exhibit IC₅₀ values below 200 nM, and even lower than 160 nM. Advantageously, most of the compounds exhibit IC₅₀ values below 100 nM, and even lower than 50 nM.

All the compounds according to the disclosure are potent RIPK1 inhibitors as they exhibit a cell death (necroptosis) in U937 cells, with IC₅₀ lower than 1000 nM. Most of the compounds even exhibit IC₅₀ values below 500 nM, and even lower than 200 nM. Advantageously, most of the compounds exhibit IC₅₀ values below 100 nM, and even lower than or equal to 50 nM.

1. A compound of formula (I) 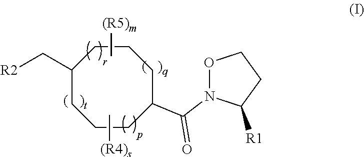
wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge;
m and s are independently 0 or 1;
p, q, r and t are independently 0 or 1;
each R6 is independently selected from halogen, cyano, —OH, a —NH₂group, a (C₁-C₄)alkyl group, —CF₃, —C(O)NH₂, a —C(O)NH—(C₁-C₄)alkyl group, —C(O)OH, a —C(O)O—(C₁-C₄)alkyl group, —SO₂NH₂, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a (C₃-C₆)heterocycloalkyl group, a heteroaryl group and oxo, wherein said (C₁-C₄)alkyl group, (C₁-C₄)alkoxy group, —O—(C₃-C₆)cycloalkyl group, (C₁-C₄)alkyl group, (C₃-C₆)heterocycloalkyl group or heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

2. The compound of formula (I) as defined in claim 1, wherein:
R1 represents a phenyl or a 5- or 6-membered monocyclic heteroaryl, which is optionally substituted by one, two or three R3;
R2 represents a phenyl, a 5- or 6-membered monocyclic heteroaryl or a 9- or 10-membered bicyclic heteroaryl, which is optionally substituted by one, two or three R6;
each R3 is independently chosen from a halogen, cyano, a (C₁-C₄)alkyl group and a (C₁-C₄)alkoxy group;
R4 and R5 form together a (C₁-C₄)alkylene bridge;
m and s are independently 0 or 1;
p, q, r and t are independently 0 or 1;
each R6 is independently selected from a halogen, cyano, a —NH₂ group, —OH, a (C₁-C₄)alkyl group, —C(O)NH₂, a —C(O)NH—(C₁-C₄)alkyl group, a (C₁-C₄)alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a 4- or 5-membered heterocycloalkyl, a 5- or 6-membered heteroaryl and oxo, wherein said (C₁-C₄)alkyl group, (C₁-C₄)alkoxy group, —O—(C₃-C₆)cycloalkyl group, heterocycloalkyl group, heteroaryl is optionally substituted by one or two R7;
each R7 is independently —OH, a (C₁-C₄)alkyl group or a (C₁-C₄)alkoxy group,
or a pharmaceutically acceptable salt, solvate, or stereoisomer thereof.

3. The compound of formula (I) as defined in claim 1, having following formula (Ig): 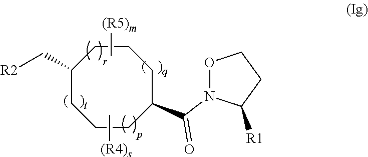
or a pharmaceutically acceptable salt, solvate, or stereoisomer thereof.

4. The compound of formula (I) as defined in claim 1, having following formula (Ia): 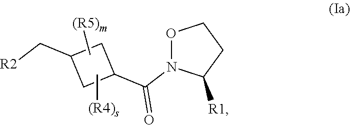
or a pharmaceutically acceptable salt, solvate, or stereoisomer thereof.

5. The compound of formula (Ia) as defined in claim 4, wherein:
R1 is a phenyl which is optionally substituted by one or two R3;
R2 is a 9-membered bicyclic heteroaryl, which is optionally substituted by one or two R6;
each R3 is independently chosen from a halogen, cyano or a (C₁-C₄)alkyl group;
m and s are 0;
each R6 is a halogen;
or a pharmaceutically acceptable salt, solvate, or stereoisomer thereof.

6. The compound of formula (Ia) as defined in claim 5, having the following formula (If): 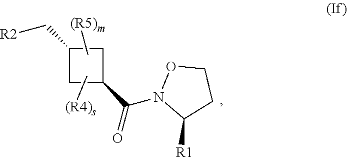
or a pharmaceutically acceptable salt, solvate, or stereoisomer thereof.

7. The compound of formula (I) as defined in claim 1, having following formula (Ib): 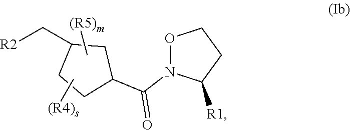
or a pharmaceutically acceptable salt, solvate, or stereoisomer thereof.

8. The compound of formula (I) as defined in claim 1, having following formula (Ic): 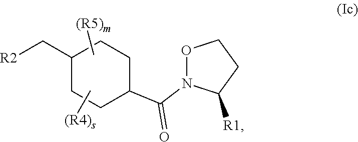
or a pharmaceutically acceptable salt, solvate, or stereoisomer thereof.

9. The compound of formula (Ic) as defined in claim 8, wherein:
R1 is phenyl or a 6-membered monocyclic heteroaryl, which is optionally substituted by one or two R3;
R2 is a 5- or 6-membered monocyclic heteroaryl or a 9-membered bicyclic heteroaryl, optionally substituted by one or two R6;
each R3 is independently chosen from a halogen, cyano, a (C₁-C₄)alkyl group, or a (C₁-C₄)alkoxy group;
m and s are 0 or m is 0 and s is 1 and R4 is a methyl group;
each R6 is independently chosen from a halogen, a cyano, a —NH₂ group, a (C₁-C₄)alkyl group, a (C₁-C₄)alkoxy group, a —CF₃, a (C₃-C₆)heterocycloalkyl group optionally substituted by one, two, three or four R7;
R7 is independently a halogen, oxo, —OH, a methyl or a methoxy;
or a pharmaceutically acceptable salt, solvate, or stereoisomer thereof.

10. The compound of formula (Ic) as defined in claim 9, having the following formula (Ie): 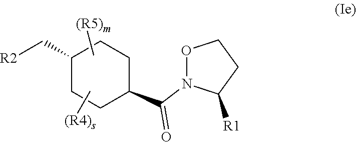
or a pharmaceutically acceptable salt, solvate, or stereoisomer thereof.

11. The compound of formula (I) as defined in claim 1, having following formula (Id): 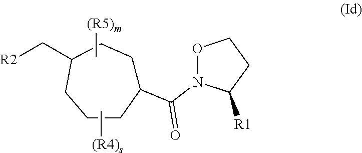
or a pharmaceutically acceptable salt, solvate, or stereoisomer thereof.

12. The compound of formula (I) as defined in claim 1, having following formula (Ib), (Ic), (Ie), or (Id): 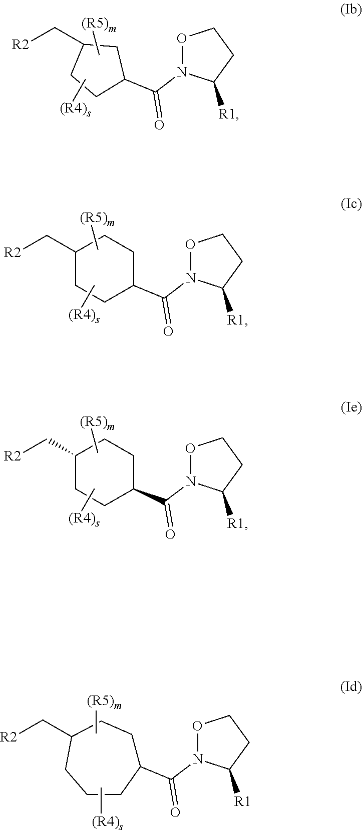
or a pharmaceutically acceptable salt, solvate, or stereoisomer thereof.

13. The compound of formula (I) as defined in claim 1, wherein when the group 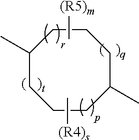
is an optionally substituted cyclobutyl, then R4 and R5 are independently chosen from a halogen, a (C₁-C₄)alkyl group or a (C₁-C₄)alkoxy group.

14. A compound or formula (I) as defined in claim 1 selected from:
(1) trans-3-[(3S)-2-[4-[(2-methyl-[1,2,4]triazolo[1,5-a]pyridin-7-yl)methyl]cyclohexanecarbonyl]isoxazolidin-3-yl]benzonitrile;
(2) trans-1-((4-((S)-3-(3-cyano-5-fluorophenyl)isoxazolidine-2-carbonyl)cyclohexyl)methyl)-1H-pyrrolo[3,2-b]pyridine-6-carbonitrile,
(3) trans-1-((4-((S)-3-(5-fluoropyridin-3-yl)isoxazolidine-2-carbonyl)cyclohexyl)methyl)-1H-pyrrolo[3,2-b]pyridine-6-carbonitrile,
(4) trans-1-((4-((S)-3-(5-fluoropyridin-3-yl)isoxazolidine-2-carbonyl)cyclohexyl)methyl)-4-methyl-1H-imidazole-5-carbonitrile,
(5) trans-1-((4-((S)-3-(3-cyano-5-fluorophenyl)isoxazolidine-2-carbonyl)cyclohexyl)methyl)-1H-benzo[d]imidazole-5-carbonitrile,
(6) trans-1-((4-((S)-3-(5-fluoropyridin-3-yl)isoxazolidine-2-carbonyl)cyclohexyl)methyl)-1H-benzo[d]imidazole-5-carbonitrile,
(7) trans-3-((S)-2-(4-((1H-benzo[d]imidazol-1-yl)methyl)cyclohexane-1-carbonyl)isoxazolidin-3-yl)-5-fluorobenzonitrile,
(8) trans-(4-((1H-benzo[d]imidazol-1-yl)methyl)cyclohexyl)((S)-3-(3,5-difluorophenyl)isoxazolidin-2-yl)methanone,
(9) trans-(4-((1H-benzo[d]imidazol-1-yl)methyl)cyclohexyl)((S)-3-(5-fluoropyridin-3-yl)isoxazolidin-2-yl)methanone,
(10) trans-1-((4-((S)-3-(3,5-difluorophenyl)isoxazolidine-2-carbonyl)cyclohexyl)methyl)-6-fluoro-1H-benzo[d]imidazole-5-carbonitrile,
(11) trans-1-((4-((S)-3-(3-cyano-5-fluorophenyl)isoxazolidine-2-carbonyl)cyclohexyl)methyl)-6-fluoro-1H-benzo[d]imidazole-5-carbonitrile,
(12) trans-1-((4-((S)-3-(3,5-difluorophenyl)isoxazolidine-2-carbonyl)cyclohexyl)methyl)-5-fluoro-1H-benzo[d]imidazole-6-carbonitrile,
(13) trans-1-((4-((S)-3-(3-cyano-5-fluorophenyl)isoxazolidine-2-carbonyl)cyclohexyl)methyl)-5-fluoro-1H-benzo[d]imidazole-6-carbonitrile,
(14) trans-3-((S)-2-(4-((1H-pyrrolo[3,2-b]pyridin-1-yl)methyl)cyclohexane-1-carbonyl)isoxazolidin-3-yl)-5-fluorobenzonitrile,
(15) trans-3-((S)-2-(4-((1H-pyrrolo[3,2-c]pyridin-1-yl)methyl)cyclohexane-1-carbonyl)isoxazolidin-3-yl)-5-fluorobenzonitrile,
(16) trans-3-((S)-2-(4-((1H-pyrrolo[2,3-c]pyridin-1-yl)methyl)cyclohexane-1-carbonyl)isoxazolidin-3-yl)-5-fluorobenzonitrile,
(17) trans-1-((4-((S)-3-(3,5-difluorophenyl)isoxazolidine-2-carbonyl)cyclohexyl)methyl)-1H-pyrrolo[3,2-b]pyridine-6-carbonitrile,
(18) trans-3-fluoro-5-((S)-2-(4-((2-oxobenzo[d]oxazol-3(2H)-yl)methyl)cyclohexane-1-carbonyl)isoxazolidin-3-yl)benzonitrile,
(19) trans-3-((S)-2-(4-((6-chloro-1H-pyrrolo[3,2-b]pyridin-1-yl)methyl)cyclohexane-1-carbonyl)isoxazolidin-3-yl)-5-fluorobenzonitrile,
(20) trans-3-((4-((S)-3-(3,5-difluorophenyl)isoxazolidine-2-carbonyl)cyclohexyl)methyl)benzo[d]oxazol-2(3H)-one,
(21) trans-(4-((6-chloro-1H-pyrrolo[3,2-b]pyridin-1-yl)methyl)cyclohexyl)((S)-3-(3,5-difluorophenyl)isoxazolidin-2-yl)methanone,
(22) trans-1-((4-((S)-3-(3-cyano-5-fluorophenyl)isoxazolidine-2-carbonyl)cyclohexyl)methyl)-5-fluoro-1H-indole-6-carbonitrile,
(23) trans-1-((4-((S)-3-(3,5-difluorophenyl)isoxazolidine-2-carbonyl)cyclohexyl)methyl)-5-fluoro-1H-indole-6-carbonitrile,
(24) trans-2-((4-((S)-3-(3-cyano-5-fluorophenyl)isoxazolidine-2-carbonyl)cyclohexyl)methyl)-2H-pyrazolo[4,3-b]pyridine-6-carbonitrile,
(25) trans-2-((4-((S)-3-(3,5-difluorophenyl)isoxazolidine-2-carbonyl)cyclohexyl)methyl)-2H-pyrazolo[4,3-b]pyridine-6-carbonitrile,
(26) trans-1-((4-((S)-3-(3,5-difluorophenyl)isoxazolidine-2-carbonyl)cyclohexyl)methyl)-1H-pyrazolo[4,3-b]pyridine-6-carbonitrile,
(27) trans-1-((4-((S)-3-(3-cyano-5-fluorophenyl)isoxazolidine-2-carbonyl)cyclohexyl)methyl)-1H-pyrazolo[4,3-b]pyridine-6-carbonitrile,
(28) trans-1-((4-((S)-3-(3-cyano-5-fluorophenyl)isoxazolidine-2-carbonyl)cyclohexyl)methyl)-1H-indazole-6-carbonitrile,
(29) trans-1-((4-((S)-3-(3,5-difluorophenyl)isoxazolidine-2-carbonyl)cyclohexyl)methyl)-1H-indazole-6-carbonitrile,
(30) trans-2-((4-((S)-3-(3-cyano-5-fluorophenyl)isoxazolidine-2-carbonyl)cyclohexyl)methyl)-2H-indazole-6-carbonitrile,
(31) trans-2-((4-((S)-3-(3,5-difluorophenyl)isoxazolidine-2-carbonyl)cyclohexyl)methyl)-2H-indazole-6-carbonitrile,
(32) trans-1-((4-((S)-3-(3-cyano-5-fluorophenyl)isoxazolidine-2-carbonyl)cyclohexyl)methyl)-4-fluoro-1H-indazole-6-carbonitrile,
(33) trans-1-((4-((S)-3-(3,5-difluorophenyl)isoxazolidine-2-carbonyl)cyclohexyl)methyl)-4-fluoro-1H-indazole-6-carbonitrile,
(34) trans-2-((4-((S)-3-(3-cyano-5-fluorophenyl)isoxazolidine-2-carbonyl)cyclohexyl)methyl)-4-fluoro-2H-indazole-6-carbonitrile,
(35) trans-2-((4-((S)-3-(3,5-difluorophenyl)isoxazolidine-2-carbonyl)cyclohexyl)methyl)-4-fluoro-2H-indazole-6-carbonitrile,
(36) trans-1-((4-((S)-3-(3-cyano-5-fluorophenyl)isoxazolidine-2-carbonyl)cyclohexyl)methyl)-4-fluoro-1H-benzo[d]imidazole-6-carbonitrile,
(37) trans-1-((4-((S)-3-(3,5-difluorophenyl)isoxazolidine-2-carbonyl)cyclohexyl)methyl)-4-fluoro-1H-benzo[d]imidazole-6-carbonitrile,
(38) trans-4-fluoro-1-((4-((S)-3-(5-methylfuran-3-yl)isoxazolidine-2-carbonyl)cyclohexyl)methyl)-1H-benzo[d]imidazole-6-carbonitrile,
(39) trans-5-fluoro-1-((4-((S)-3-(5-methylfuran-3-yl)isoxazolidine-2-carbonyl)cyclohexyl)methyl)-1H-benzo[d]imidazole-6-carbonitrile,
(40) trans-6-fluoro-1-((4-((S)-3-(5-methylfuran-3-yl)isoxazolidine-2-carbonyl)cyclohexyl)methyl)-1H-benzo[d]imidazole-5-carbonitrile,
(41) trans-2-((4-((S)-3-(5-methylfuran-3-yl)isoxazolidine-2-carbonyl)cyclohexyl)methyl)-2H-indazole-6-carbonitrile,
(42) trans-4-fluoro-2-((4-((S)-3-(5-methylfuran-3-yl)isoxazolidine-2-carbonyl)cyclohexyl)methyl)-2H-indazole-6-carbonitrile,
(43) trans-1-((4-((S)-3-(5-methylfuran-3-yl)isoxazolidine-2-carbonyl)cyclohexyl)methyl)-1H-indazole-6-carbonitrile,
(44) trans-4-fluoro-1-((4-((S)-3-(5-methylfuran-3-yl)isoxazolidine-2-carbonyl)cyclohexyl)methyl)-1H-indazole-6-carbonitrile,
(45) trans-1-((4-((S)-3-(3,5-difluorophenyl)isoxazolidine-2-carbonyl)cyclohexyl)methyl)-4-fluoro-1H-indole-6-carbonitrile,
(46) trans-1-((4-((S)-3-(3-cyano-5-fluorophenyl)isoxazolidine-2-carbonyl)cyclohexyl)methyl)-4-fluoro-1H-indole-6-carbonitrile,
(47) trans-4-fluoro-1-((4-((S)-3-(5-methylfuran-3-yl)isoxazolidine-2-carbonyl)cyclohexyl)methyl)-1H-indole-6-carbonitrile,
(48) trans-3-fluoro-5-((S)-2-(4-((5-fluoro-1H-benzo[d]imidazol-1-yl)methyl)cyclohexane-1-carbonyl)isoxazolidin-3-yl)benzonitrile,
(49) trans-((S)-3-(3,5-difluorophenyl)isoxazolidin-2-yl)(4-((5-fluoro-1H-benzo[d]imidazol-1-yl)methyl)cyclohexyl)methanone,
(50) trans-3-fluoro-5-((S)-2-(4-((5-fluoro-2-methyl-1H-benzo[d]imidazol-1-yl)methyl)cyclohexane-1-carbonyl)isoxazolidin-3-yl)benzonitrile,
(51) trans-((S)-3-(3,5-difluorophenyl)isoxazolidin-2-yl)(4-((5-fluoro-2-methyl-1H-benzo[d]imidazol-1-yl)methyl)cyclohexyl)methanone,
(52) trans-1-((4-((S)-3-(3,5-difluorophenyl)isoxazolidine-2-carbonyl)cyclohexyl)methyl)-1H-indazole-5-carbonitrile,
(53) trans-1-((4-((S)-3-(3-cyano-5-fluorophenyl)isoxazolidine-2-carbonyl)cyclohexyl)methyl)-1H-indazole-5-carbonitrile,
(54) trans-3-fluoro-5-((S)-2-(4-((5-fluoro-1H-indazol-1-yl)methyl)cyclohexane-1-carbonyl)isoxazolidin-3-yl)benzonitrile,
(55) trans-((S)-3-(3,5-difluorophenyl)isoxazolidin-2-yl)(4-((5-fluoro-1H-indazol-1-yl)methyl)cyclohexyl)methanone,
(56) trans-3-fluoro-5-((S)-2-(4-((5-fluoro-2H-indazol-2-yl)methyl)cyclohexane-1-carbonyl)isoxazolidin-3-yl)benzonitrile,
(57) trans-((S)-3-(3,5-difluorophenyl)isoxazolidin-2-yl)(4-((5-fluoro-2H-indazol-2-yl)methyl)cyclohexyl)methanone,
(58) trans-2-((4-((S)-3-(3,5-difluorophenyl)isoxazolidine-2-carbonyl)cyclohexyl)methyl)-2H-indazole-5-carbonitrile,
(59) trans-2-((4-((S)-3-(3-cyano-5-fluorophenyl)isoxazolidine-2-carbonyl)cyclohexyl)methyl)-2H-indazole-5-carbonitrile,
(60) trans-((S)-3-(3,5-difluorophenyl)isoxazolidin-2-yl)(4-((3-methyl-1H-indazol-1-yl)methyl)cyclohexyl)methanone,
(61) trans-3-fluoro-5-((S)-2-(4-((3-methyl-1H-indazol-1-yl)methyl)cyclohexane-1-carbonyl)isoxazolidin-3-yl)benzonitrile,
(62) trans-((S)-3-(3,5-difluorophenyl)isoxazolidin-2-yl)(4-((3-methyl-2H-indazol-2-yl)methyl)cyclohexyl)methanone,
(63) trans-3-fluoro-5-((S)-2-(4-((3-methyl-2H-indazol-2-yl)methyl)cyclohexane-1-carbonyl)isoxazolidin-3-yl)benzonitrile,
(64) trans-1-((4-((S)-3-(5-cyanopyridin-3-yl)isoxazolidine-2-carbonyl)cyclohexyl)methyl)-5-fluoro-1H-benzo[d]imidazole-6-carbonitrile,
(65) trans-1-((4-((S)-3-(5-cyanopyridin-3-yl)isoxazolidine-2-carbonyl)cyclohexyl)methyl)-6-fluoro-1H-benzo[d]imidazole-5-carbonitrile,
(66) trans-1-((4-((S)-3-(3-cyano-5-fluorophenyl)isoxazolidine-2-carbonyl)cyclohexyl)methyl)-6-fluoro-1H-indazole-5-carbonitrile,
(67) trans-1-((4-((S)-3-(3,5-difluorophenyl)isoxazolidine-2-carbonyl)cyclohexyl)methyl)-6-fluoro-1H-indazole-5-carbonitrile,
(68) trans-1-((4-((S)-3-(3,5-difluorophenyl)isoxazolidine-2-carbonyl)cyclohexyl)methyl)-6-fluoro-3-methyl-1H-indazole-5-carbonitrile,
(69) trans-1-((4-((S)-3-(3-cyano-5-fluorophenyl)isoxazolidine-2-carbonyl)cyclohexyl)methyl)-6-fluoro-3-methyl-1H-indazole-5-carbonitrile,
(70) trans-1-((4-((S)-3-(3-cyano-5-fluorophenyl)isoxazolidine-2-carbonyl)cyclohexyl)methyl)-5-fluoro-1H-benzo[d]imidazole-6-carboxamide,
(71) trans-1-((4-((S)-3-(3,5-difluorophenyl)isoxazolidine-2-carbonyl)cyclohexyl)methyl)-5-fluoro-1H-benzo[d]imidazole-6-carboxamide,
(72) trans-1-((4-((S)-3-(3-cyano-5-fluorophenyl)isoxazolidine-2-carbonyl)cyclohexyl)methyl)-1H-indazole-5-carboxamide,
(73) trans-1-((4-((S)-3-(3,5-difluorophenyl)isoxazolidine-2-carbonyl)cyclohexyl)methyl)-1H-indazole-5-carboxamide,
(74) trans-1-((4-((S)-3-(3-cyano-5-fluorophenyl)isoxazolidine-2-carbonyl)cyclohexyl)methyl)-6-fluoro-1H-benzo[d]imidazole-5-carboxamide,
(75) trans-1-((4-((S)-3-(3,5-difluorophenyl)isoxazolidine-2-carbonyl)cyclohexyl)methyl)-6-fluoro-1H-benzo[d]imidazole-5-carboxamide,
(76) trans-1-((4-((S)-3-(3-cyano-5-fluorophenyl)isoxazolidine-2-carbonyl)cyclohexyl)methyl)-5-fluoro-1H-indole-6-carboxamide,
(77) trans-1-((4-((S)-3-(3,5-difluorophenyl)isoxazolidine-2-carbonyl)cyclohexyl)methyl)-5-fluoro-1H-indole-6-carboxamide,
(78) trans-1-((4-((S)-3-(3-cyano-5-fluorophenyl)isoxazolidine-2-carbonyl)cyclohexyl)methyl)-1H-pyrazolo[4,3-b]pyridine-6-carboxamide,
(79) trans-1-((4-((S)-3-(3,5-difluorophenyl)isoxazolidine-2-carbonyl)cyclohexyl)methyl)-1H-pyrazolo[4,3-b]pyridine-6-carboxamide,
(80) trans-1-((4-((S)-3-(3-cyano-5-fluorophenyl)isoxazolidine-2-carbonyl)cyclohexyl)methyl)-4-fluoro-1H-benzo[d]imidazole-6-carboxamide,
(81) trans-1-((4-((S)-3-(3,5-difluorophenyl)isoxazolidine-2-carbonyl)cyclohexyl)methyl)-4-fluoro-1H-benzo[d]imidazole-6-carboxamide,
(82) trans-3-((S)-2-(4-((4,5-dimethyl-1H-imidazol-1-yl)methyl)cyclohexane-1-carbonyl)isoxazolidin-3-yl)-5-fluorobenzonitrile,
(83) trans-((S)-3-(3,5-difluorophenyl)isoxazolidin-2-yl)(4-((4,5-dimethyl-1H-imidazol-1-yl)methyl)cyclohexyl)methanone,
(84) trans-1-((4-((S)-3-(3,5-difluorophenyl)isoxazolidine-2-carbonyl)cyclohexyl)methyl)-5-fluoro-3-methyl-1H-indazole-6-carbonitrile,
(85) trans-1-((4-((S)-3-(3-cyano-5-fluorophenyl)isoxazolidine-2-carbonyl)cyclohexyl)methyl)-5-fluoro-3-methyl-1H-indazole-6-carbonitrile,
(86) trans-1-((4-((S)-3-(3,5-difluorophenyl)isoxazolidine-2-carbonyl)cyclohexyl)methyl)-5-fluoro-1H-indazole-6-carbonitrile,
(87) trans-1-((4-((S)-3-(3-cyano-5-fluorophenyl)isoxazolidine-2-carbonyl)cyclohexyl)methyl)-5-fluoro-1H-indazole-6-carbonitrile,
(88) trans-1-((4-((S)-3-(3-cyano-5-fluorophenyl)isoxazolidine-2-carbonyl)cyclohexyl)methyl)-4-methyl-1H-imidazole-5-carbonitrile,
(89) trans-1-((4-((S)-3-(3,5-difluorophenyl)isoxazolidine-2-carbonyl)cyclohexyl)methyl)-4-methyl-1H-imidazole-5-carbonitrile,
(90) trans-1-((4-((S)-3-(3-cyano-5-fluorophenyl)isoxazolidine-2-carbonyl)cyclohexyl)methyl)-5-methyl-1H-imidazole-4-carbonitrile,
(91) trans-1-((4-((S)-3-(3,5-difluorophenyl)isoxazolidine-2-carbonyl)cyclohexyl)methyl)-5-methyl-1H-imidazole-4-carbonitrile,
(92) trans-(4-((5-fluoro-1H-benzo[d]imidazol-1-yl)methyl)cyclohexyl)((S)-3-(pyrazin-2-yl)isoxazolidin-2-yl)methanone,
(93) trans-3-fluoro-5-((S)-2-(4-((6-fluoro-1H-pyrrolo[3,2-b]pyridin-1-yl)methyl)cyclohexane-1-carbonyl)isoxazolidin-3-yl)benzonitrile,
(94) trans-((S)-3-(3,5-difluorophenyl)isoxazolidin-2-yl)(4-((6-fluoro-1H-pyrrolo[3,2-b]pyridin-1-yl)methyl)cyclohexyl)methanone,
(95) trans-3-fluoro-5-((S)-2-(4-((5-fluoro-1H-indol-1-yl)methyl)cyclohexane-1-carbonyl)isoxazolidin-3-yl)benzonitrile,
(96) trans-((S)-3-(3,5-difluorophenyl)isoxazolidin-2-yl)(4-((5-fluoro-1H-indol-1-yl)methyl)cyclohexyl)methanone,
(97) trans-6-fluoro-1-((4-((S)-3-(pyrazin-2-yl)isoxazolidine-2-carbonyl)cyclohexyl)methyl)-1H-benzo[d]imidazole-5-carbonitrile,
(98) trans-5-fluoro-1-((4-((S)-3-(pyrazin-2-yl)isoxazolidine-2-carbonyl)cyclohexyl)methyl)-1H-benzo[d]imidazole-6-carbonitrile,
(99) trans-1-[[4-[(3S)-3-(3-cyano-5-fluoro-phenyl)isoxazolidine-2-carbonyl]cyclohexyl]methyl]pyrrolo[3,2-b]pyridine-5-carbonitrile,
(100) trans-1-((4-((S)-3-(3-cyano-5-fluorophenyl)isoxazolidine-2-carbonyl)cyclohexyl)methyl)-4-fluoro-3-methyl-1H-indazole-6-carbonitrile,
(101) trans-1-((4-((S)-3-(3,5-difluorophenyl)isoxazolidine-2-carbonyl)cyclohexyl)methyl)-4-fluoro-3-methyl-1H-indazole-6-carbonitrile,
(102) trans-1-((4-((S)-3-(3-cyano-5-fluorophenyl)isoxazolidine-2-carbonyl)cyclohexyl)methyl)-4-fluoro-3-methyl-1H-indazole-6-carboxamide,
(103) trans-1-((4-((S)-3-(3,5-difluorophenyl)isoxazolidine-2-carbonyl)cyclohexyl)methyl)-4-fluoro-3-methyl-1H-indazole-6-carboxamide,
(104) trans-1-((4-((S)-3-(3-cyano-5-fluorophenyl)isoxazolidine-2-carbonyl)cyclohexyl)methyl)-1H-indole-6-carboxamide,
(105) trans-1-((4-((S)-3-(3,5-difluorophenyl)isoxazolidine-2-carbonyl)cyclohexyl)methyl)-1H-indole-6-carboxamide,
(106) trans-5-((S)-2-(4-((6-fluoro-1H-benzo[d]imidazol-1-yl)methyl)cyclohexane-1-carbonyl)isoxazolidin-3-yl)nicotinonitrile,
(107) trans-5-((S)-2-(4-((5-fluoro-1H-benzo[d]imidazol-1-yl)methyl)cyclohexane-1-carbonyl)isoxazolidin-3-yl)nicotinonitrile,
(108) trans-1-((4-((S)-3-(5-cyanopyridin-3-yl)isoxazolidine-2-carbonyl)cyclohexyl)methyl)-1H-pyrrolo[3,2-b]pyridine-6-carbonitrile,
(109) trans-5-((4-((S)-3-(3-cyano-5-fluorophenyl)isoxazolidine-2-carbonyl)cyclohexyl)methyl)-2-methylbenzonitrile,
(110) trans-1-((4-((S)-3-(3-cyano-5-fluorophenyl)isoxazolidine-2-carbonyl)cyclohexyl)methyl)-3-methyl-1H-indole-6-carboxamide,
(111) trans-1-((4-((S)-3-(3,5-difluorophenyl)isoxazolidine-2-carbonyl)cyclohexyl)methyl)-3-methyl-1H-indole-6-carboxamide,
(112) trans-3-fluoro-5-((S)-2-(4-((6-fluoro-1H-benzo[d]imidazol-1-yl)methyl)cyclohexane-1-carbonyl)isoxazolidin-3-yl)benzonitrile,
(113) trans-((S)-3-(3,5-difluorophenyl)isoxazolidin-2-yl)(4-((6-fluoro-1H-benzo[d]imidazol-1-yl)methyl)cyclohexyl)methanone,
(114) trans-(3-((5-fluoro-1H-benzo[d]imidazol-1-yl)methyl)cyclobutyl)((S)-3-(p-tolyl)isoxazolidin-2-yl)methanone,
(115) trans-((S)-3-(3,5-difluorophenyl)isoxazolidin-2-yl)(3-((5-fluoro-1H-benzo[d]imidazol-1-yl)methyl)cyclobutyl)methanone,
(116) trans-3-fluoro-5-((S)-2-(3-((5-fluoro-1H-benzo[d]imidazol-1-yl)methyl)cyclobutane-1-carbonyl)isoxazolidin-3-yl)benzonitrile,
(117) trans-(4-((3,5-dimethyl-1H-pyrazol-1-yl)methyl)cyclohexyl)((S)-3-(p-tolyl)isoxazolidin-2-yl)methanone,
(118) trans-((S)-3-(3,5-difluorophenyl)isoxazolidin-2-yl)(4-((3,5-dimethyl-1H-pyrazol-1-yl)methyl)cyclohexyl)methanone,
(119) trans-3-((S)-2-(4-((3,5-dimethyl-1H-pyrazol-1-yl)methyl)cyclohexane-1-carbonyl)isoxazolidin-3-yl)-5-fluorobenzonitrile,
(120) trans-3-fluoro-5-[(3S)-2-[4-[(5-methoxypyrrolo[3,2-b]pyridin-1-yl)methyl]cyclohexanecarbonyl]isoxazolidin-3-yl]benzonitrile,
(121) trans-2-((4-((S)-3-(5-cyanopyridin-3-yl)isoxazolidine-2-carbonyl)cyclohexyl)methyl)-2H-pyrazolo[3,4-b]pyridine-5-carbonitrile,
(122) trans-1-[[4-[(3S)-3-(5-cyano-3-pyridyl)isoxazolidine-2-carbonyl]cyclohexyl]methyl]-6-oxo-pyridine-3-carbonitrile,
(123) trans-5-((S)-2-(4-((6-fluoro-1H-pyrrolo[3,2-b]pyridin-1-yl)methyl)cyclohexane-1-carbonyl)isoxazolidin-3-yl)nicotinonitrile,
(124) trans-3-[(3S)-2-[4-[[6-(3,5-dimethylpyrazol-1-yl)pyrimidin-4-yl]methyl]cyclohexanecarbonyl]isoxazolidin-3-yl]-5-fluoro-benzonitrile,
(125) trans-[(3S)-3-(3,5-difluorophenyl)isoxazolidin-2-yl]-[4-[[6-(3,5-dimethylpyrazol-1-yl)pyrimidin-4-yl]methyl]cyclohexyl]methanone,
(126) trans-5-((S)-2-(4-((5-fluoro-1H-indazol-1-yl)methyl)cyclohexane-1-carbonyl)isoxazolidin-3-yl)nicotinonitrile,
(127) trans-2-((4-((S)-3-(5-cyanopyridin-3-yl)isoxazolidine-2-carbonyl)cyclohexyl)methyl)-2H-indazole-6-carbonitrile,
(128) trans-3-fluoro-5-[(3S)-2-[4-[(6-methoxypyrrolo[3,2-b]pyridin-1-yl)methyl]cyclohexanecarbonyl]isoxazolidin-3-yl]benzonitrile,
(129) trans-3-fluoro-5-[(3S)-2-[4-[(6-hydroxypyrrolo[3,2-b]pyridin-1-yl)methyl]cyclohexanecarbonyl]isoxazolidin-3-yl]benzonitrile,
(130) trans-1-((4-((S)-3-(5-cyanopyridin-3-yl)isoxazolidine-2-carbonyl)cyclohexyl)methyl)-4-fluoro-1H-indole-6-carbonitrile,
(131) trans-2-methyl-5-[[4-[(3S)-3-(p-tolyl)isoxazolidine-2-carbonyl]cyclohexyl]methyl]benzamide,
(132) trans-5-[[4-[(3S)-3-(3,5-difluorophenyl)isoxazolidine-2-carbonyl]cyclohexyl]methyl]-2-methyl-benzamide,
(133) trans-5-[[4-[(3S)-3-(3-cyano-5-fluoro-phenyl)isoxazolidine-2-carbonyl]cyclohexyl]methyl]-2-methyl-benzamide,
(134) trans-1-[[4-[(3S)-3-pyrazin-2-ylisoxazolidine-2-carbonyl]cyclohexyl]methyl]pyrrolo[3,2-b]pyridine-6-carbonitrile,
(135) trans-3-fluoro-5-[(3S)-2-[4-[[6-(2-methylimidazol-1-yl)pyrimidin-4-yl]methyl]cyclohexanecarbonyl]isoxazolidin-3-yl]benzonitrile,
(136) trans-[4-[[6-(2-methylimidazol-1-yl)pyrimidin-4-yl]methyl]cyclohexyl]-[(3S)-3-(p-tolyl)isoxazolidin-2-yl]methanone,
(137) trans-1-[[4-[(3S)-3-pyrazin-2-ylisoxazolidine-2-carbonyl]cyclohexyl]methyl]pyrazolo[4,3-b]pyridine-6-carbonitrile,
(138) trans-2-methyl-5-[[4-[(3S)-3-pyrazin-2-ylisoxazolidine-2-carbonyl]cyclohexyl]methyl]benzamide,
(139) trans-2-methyl-5-[[4-[(3S)-3-pyrazin-2-ylisoxazolidine-2-carbonyl]cyclohexyl]methyl]benzonitrile,
(140) trans-5-[[4-[(3S)-3-pyrazin-2-ylisoxazolidine-2-carbonyl]cyclohexyl]methyl]pyridine-3-carbonitrile,
(141) trans-5-[[4-[(3S)-3-(3-cyano-5-fluoro-phenyl)isoxazolidine-2-carbonyl]cyclohexyl]methyl]pyridine-3-carbonitrile,
(142) trans-3-fluoro-5-[(3S)-2-[4-([1,2,4]triazolo[1,5-a]pyridin-6-ylmethyl)cyclohexanecarbonyl]isoxazolidin-3-yl]benzonitrile,
(143) trans-[(3S)-3-pyrazin-2-ylisoxazolidin-2-yl]-[4-([1,2,4]triazolo[1,5-a]pyridin-6-ylmethyl)cyclohexyl]methanone,
(144) trans-3-fluoro-5-[(3S)-2-[4-[(3-oxoisoindolin-5-yl)methyl]cyclohexanecarbonyl]isoxazolidin-3-yl]benzonitrile,
(145) trans-6-[[4-[(3S)-3-pyrazin-2-ylisoxazolidine-2-carbonyl]cyclohexyl]methyl]isoindolin-1-one,
(146) trans-3-fluoro-5-[(3S)-2-[4-[[6-(oxetan-3-yloxy)pyrrolo[3,2-b]pyridin-1-yl]methyl]cyclohexanecarbonyl]isoxazolidin-3-yl]benzonitrile,
(147) trans-[4-[(5-fluoro-3-pyridyl)methyl]cyclohexyl]-[(3S)-3-pyrazin-2-ylisoxazolidin-2-yl]methanone,
(148) trans-3-fluoro-5-[(3S)-2-[4-[(5-fluoro-3-pyridyl)methyl]cyclohexanecarbonyl]isoxazolidin-3-yl]benzonitrile,
(149) trans-3-fluoro-5-[(3S)-2-[4-[[6-(2-methoxyethoxy)pyrrolo[3,2-b]pyridin-1-yl]methyl]cyclohexanecarbonyl]isoxazolidin-3-yl]benzonitrile,
(150) trans-3-fluoro-5-[(3S)-2-[4-[(1-methylpyrazol-4-yl)methyl]cyclohexanecarbonyl]isoxazolidin-3-yl]benzonitrile,
(151) trans-[4-[(1-methylpyrazol-4-yl)methyl]cyclohexyl]-[(3S)-3-pyrazin-2-ylisoxazolidin-2-yl]methanone,
(152) trans-3-[(3S)-2-[4-[[6-(cyclopropylmethoxy)pyrrolo[3,2-b]pyridin-1-yl]methyl]cyclohexanecarbonyl]isoxazolidin-3-yl]-5-fluoro-benzonitrile,
(153) trans-3-fluoro-5-[(3S)-2-[4-[[6-(2-hydroxyethoxy)pyrrolo[3,2-b]pyridin-1-yl]methyl]cyclohexanecarbonyl]isoxazolidin-3-yl]benzonitrile,
(154) trans-6-[[4-[(3S)-3-(4-fluorophenyl)isoxazolidine-2-carbonyl]cyclohexyl]methyl]isoindolin-1-one,
(155) trans-6-[[4-[(3S)-3-(5-fluoro-3-pyridyl)isoxazolidine-2-carbonyl]cyclohexyl]methyl]isoindolin-1-one,
(156) trans-6-[[4-[(3S)-3-(3,5-difluorophenyl)isoxazolidine-2-carbonyl]cyclohexyl]methyl]isoindolin-1-one,
(157) trans-6-[[4-[(3S)-3-(6-methyl-3-pyridyl)isoxazolidine-2-carbonyl]cyclohexyl]methyl]isoindolin-1-one,
(158) trans-5-[[4-[(3S)-3-(5-cyano-3-pyridyl)isoxazolidine-2-carbonyl]cyclohexyl]methyl]-2-methyl-benzamide,
(159) trans-2-methyl-5-[[4-[(3S)-3-(2-methylthiazol-4-yl)isoxazolidine-2-carbonyl]cyclohexyl]methyl]benzamide,
(160) trans-3-fluoro-5-[[4-[(3S)-3-(5-fluoro-3-pyridyl)isoxazolidine-2-carbonyl]cyclohexyl]methyl]-N-methyl-benzamide,
(161) trans-3-[[4-[(3S)-3-(3,5-difluorophenyl)isoxazolidine-2-carbonyl]cyclohexyl]methyl]-5-fluoro-N-methyl-benzamide,
(162) trans-3-fluoro-N-methyl-5-[[4-[(3S)-3-pyrazin-2-ylisoxazolidine-2-carbonyl]cyclohexyl]methyl]benzamide,
(163) trans-3-[[4-[(3S)-3-(5-cyano-3-pyridyl)isoxazolidine-2-carbonyl]cyclohexyl]methyl]-5-fluoro-N-methyl-benzamide,
(164) trans-3-[[4-[(3S)-3-(3-cyano-5-fluoro-phenyl)isoxazolidine-2-carbonyl]cyclohexyl]methyl]-5-fluoro-N-methyl-benzamide,
(165) trans-3-fluoro-5-[[4-[(3S)-3-(4-fluorophenyl)isoxazolidine-2-carbonyl]cyclohexyl]methyl]-N-methyl-benzamide,
(166) trans-5-[[4-[(3S)-3-(6-methoxypyrazin-2-yl)isoxazolidine-2-carbonyl]cyclohexyl]methyl]-2-methyl-benzamide,
(167) trans-3-fluoro-5-[(3S)-2-[4-[(3-methylimidazol-4-yl)methyl]cyclohexanecarbonyl]isoxazolidin-3-yl]benzonitrile,
(168) trans-5-[(3S)-2-[4-[(3-methylimidazol-4-yl)methyl]cyclohexanecarbonyl]isoxazolidin-3-yl]pyridine-3-carbonitrile,
(169) trans-3-fluoro-5-[(3S)-2-[4-[[4-(3-hydroxyoxetan-3-yl)phenyl]methyl]cyclohexanecarbonyl]isoxazolidin-3-yl]benzonitrile,
(170) trans-[(3S)-3-(4-fluorophenyl)isoxazolidin-2-yl]-[4-[[4-(3-hydroxyoxetan-3-yl)phenyl]methyl]cyclohexyl]methanone,
(171) trans-[(3S)-3-(3,5-difluorophenyl)isoxazolidin-2-yl]-[4-[[4-(3-hydroxyoxetan-3-yl)phenyl]methyl]cyclohexyl]methanone,
(172) trans-4-fluoro-3-[[4-[(3S)-3-(5-fluoro-3-pyridyl)isoxazolidine-2-carbonyl]cyclohexyl]methyl]-N-methyl-benzamide,
(173) trans-4-fluoro-3-[[4-[(3S)-3-(4-fluorophenyl)isoxazolidine-2-carbonyl]cyclohexyl]methyl]-N-methyl-benzamide,
(174) trans-3-[[4-[(3S)-3-(3,5-difluorophenyl)isoxazolidine-2-carbonyl]cyclohexyl]methyl]-4-fluoro-N-methyl-benzamide,
(175) trans-4-fluoro-N-methyl-3-[[4-[(3S)-3-pyrazin-2-ylisoxazolidine-2-carbonyl]cyclohexyl]methyl]benzamide,
(176) trans-3-[[4-[(3S)-3-(5-cyano-3-pyridyl)isoxazolidine-2-carbonyl]cyclohexyl]methyl]-4-fluoro-N-methyl-benzamide,
(177) trans-3-[[4-[(3S)-3-(3-cyano-5-fluoro-phenyl)isoxazolidine-2-carbonyl]cyclohexyl]methyl]-4-fluoro-N-methyl-benzamide,
(178) trans-[4-[(5-fluoro-2-methyl-3-pyridyl)methyl]cyclohexyl]-[(3S)-3-(5-fluoro-3-pyridyl)isoxazolidin-2-yl]methanone,
(179) trans-[4-[(5-fluoro-2-methyl-3-pyridyl)methyl]cyclohexyl]-[(3S)-3-(4-fluorophenyl)isoxazolidin-2-yl]methanone,
(180) trans-[4-[(5-fluoro-2-methyl-3-pyridyl)methyl]cyclohexyl]-[(3S)-3-pyrazin-2-ylisoxazolidin-2-yl]methanone,
(181) trans-3-fluoro-5-[(3S)-2-[4-[(5-fluoro-2-methyl-3-pyridyl)methyl]cyclohexanecarbonyl]isoxazolidin-3-yl]benzonitrile,
(182) trans-[(3S)-3-(3,5-difluorophenyl)isoxazolidin-2-yl]-[4-[(5-fluoro-2-methyl-3-pyridyl)methyl]cyclohexyl]methanone,
(183) trans-5-[(3S)-2-[4-[[4-(3-hydroxyoxetan-3-yl)phenyl]methyl]cyclohexanecarbonyl]isoxazolidin-3-yl]pyridine-3-carbonitrile,
(184) trans-3-fluoro-5-[(3S)-2-[4-[[6-(1-methylcyclopropoxy)pyrrolo[3,2-b]pyridin-1-yl]methyl]cyclohexanecarbonyl]isoxazolidin-3-yl]benzonitrile,
(185) trans-1-[[4-[(3S)-3-(6-methoxypyrazin-2-yl)isoxazolidine-2-carbonyl]cyclohexyl]methyl]pyrrolo[3,2-b]pyridine-6-carbonitrile,
(186) trans-1-[[4-[(3S)-3-(6-methoxypyrazin-2-yl)isoxazolidine-2-carbonyl]cyclohexyl]methyl]pyrazolo[4,3-b]pyridine-6-carbonitrile,
(187) trans-1-[[4-[(3S)-3-(2-pyridyl)isoxazolidine-2-carbonyl]cyclohexyl]methyl]pyrazolo[4,3-b]pyridine-6-carbonitrile,
(188) trans-1-[[4-[(3S)-3-(2-pyridyl)isoxazolidine-2-carbonyl]cyclohexyl]methyl]pyrrolo[3,2-b]pyridine-6-carbonitrile,
(189) trans-2-[[4-[(3S)-3-(6-methoxypyrazin-2-yl)isoxazolidine-2-carbonyl]cyclohexyl]methyl]indazole-6-carbonitrile,
(190) trans-1-[[4-[(3S)-3-(6-methoxypyrazin-2-yl)isoxazolidine-2-carbonyl]cyclohexyl]methyl]indazole-6-carbonitrile,
(191) trans-2-fluoro-5-[[4-[(3S)-3-(4-fluorophenyl)isoxazolidine-2-carbonyl]cyclohexyl]methyl]benzamide,
(192) trans-5-[[4-[(3S)-3-(3,5-difluorophenyl)isoxazolidine-2-carbonyl]cyclohexyl]methyl]-2-fluoro-benzamide,
(193) trans-2-fluoro-5-[[4-[(3S)-3-pyrazin-2-ylisoxazolidine-2-carbonyl]cyclohexyl]methyl]benzamide,
(194) trans-5-[[4-[(3S)-3-(5-cyano-3-pyridyl)isoxazolidine-2-carbonyl]cyclohexyl]methyl]-2-fluoro-benzamide,
(195) trans-5-[[4-[(3S)-3-(3-cyano-5-fluoro-phenyl)isoxazolidine-2-carbonyl]cyclohexyl]methyl]-2-fluoro-benzamide,
(196) trans-2-[[4-[(3S)-3-pyrazin-2-ylisoxazolidine-2-carbonyl]cyclohexyl]methyl]indazole-6-carbonitrile,
(197) trans-5-[(3S)-2-[4-[(5-fluoro-2-methyl-3-pyridyl)methyl]cyclohexanecarbonyl]isoxazolidin-3-yl]pyridine-3-carbonitrile,
(198) trans-1-[[4-[(3S)-3-(2-methylthiazol-4-yl)isoxazolidine-2-carbonyl]cyclohexyl]methyl]pyrrolo[3,2-b]pyridine-6-carbonitrile,
(199) trans-1-[[4-[(3S)-3-(2-methylthiazol-4-yl)isoxazolidine-2-carbonyl]cyclohexyl]methyl]pyrazolo[4,3-b]pyridine-6-carbonitrile,
(200) trans-1-[[4-[(3S)-3-pyrazin-2-ylisoxazolidine-2-carbonyl]cyclohexyl]methyl]indazole-6-carbonitrile,
(201) trans-1-[[4-[(3S)-3-(6-methoxypyrazin-2-yl)isoxazolidine-2-carbonyl]cyclohexyl]methyl]indazole-6-carboxamide,
(202) trans-1-[[4-[(3S)-3-(2-methylthiazol-4-yl)isoxazolidine-2-carbonyl]cyclohexyl]methyl]indazole-5-carbonitrile,
(203) trans-2-[[4-[(3S)-3-(2-methylthiazol-4-yl)isoxazolidine-2-carbonyl]cyclohexyl]methyl]indazole-6-carbonitrile,
(204) trans-1-[[4-[(3S)-3-(2-methylthiazol-4-yl)isoxazolidine-2-carbonyl]cyclohexyl]methyl]indazole-6-carbonitrile,
(205) trans-[4-[(8-fluoro-[1,2,4]triazolo[1,5-a]pyridin-6-yl)methyl]cyclohexyl]-[(3S)-3-pyrazin-2-ylisoxazolidin-2-yl]methanone,
(206) trans-3-fluoro-5-[(3S)-2-[4-[(8-fluoro-[1,2,4]triazolo[1,5-a]pyridin-6-yl)methyl]cyclohexanecarbonyl]isoxazolidin-3-yl]benzonitrile,
(207) trans-[4-[[3-fluoro-5-(2-methylimidazol-1-yl)phenyl]methyl]cyclohexyl]-[(3S)-3-(4-fluorophenyl)isoxazolidin-2-yl]methanone,
(208) trans-[(3S)-3-(3,5-difluorophenyl)isoxazolidin-2-yl]-[4-[[3-fluoro-5-(2-methylimidazol-1-yl)phenyl]methyl]cyclohexyl]methanone,
(209) trans-[4-[[3-fluoro-5-(2-methylimidazol-1-yl)phenyl]methyl]cyclohexyl]-[(3S)-3-pyrazin-2-ylisoxazolidin-2-yl]methanone,
(210) trans-5-[(3S)-2-[4-[[3-fluoro-5-(2-methylimidazol-1-yl)phenyl]methyl]cyclohexanecarbonyl]isoxazolidin-3-yl]pyridine-3-carbonitrile,
(211) trans-[4-[[3-fluoro-5-(2-methylpyrazol-3-yl)phenyl]methyl]cyclohexyl]-[(3S)-3-pyrazin-2-ylisoxazolidin-2-yl]methanone,
(212) trans-4-fluoro-6-[[4-[(3S)-3-(4-fluorophenyl)isoxazolidine-2-carbonyl]cyclohexyl]methyl]isoindolin-1-one,
(213) trans-4-fluoro-6-[[4-[(3S)-3-pyrazin-2-ylisoxazolidine-2-carbonyl]cyclohexyl]methyl]isoindolin-1-one,
(214) trans-3-fluoro-5-[(3S)-2-[4-[[3-fluoro-5-(2-methylpyrazol-3-yl)phenyl]methyl]cyclohexanecarbonyl]isoxazolidin-3-yl]benzonitrile,
(215) trans-1-[[4-[(3S)-3-(2-pyridyl)isoxazolidine-2-carbonyl]cyclohexyl]methyl]indazole-6-carbonitrile,
(216) trans-3-fluoro-5-[(3S)-2-[4-[[3-fluoro-5-(2-methylimidazol-1-yl)phenyl]methyl]cyclohexanecarbonyl]isoxazolidin-3-yl]benzonitrile,
(217) trans-1-[[4-[(3S)-3-(6-methoxypyrazin-2-yl)isoxazolidine-2-carbonyl]cyclohexyl]methyl]indazole-5-carbonitrile,
(218) trans-1-[[4-[(3S)-3-pyrazin-2-ylisoxazolidine-2-carbonyl]cyclohexyl]methyl]indazole-5-carbonitrile,
(219) trans-3-fluoro-5-[(3S)-2-[4-[(7-fluoro-3-oxo-isoindolin-5-yl)methyl]cyclohexanecarbonyl]isoxazolidin-3-yl]benzonitrile,
(220) trans-[4-[[3-fluoro-5-(2-methylimidazol-1-yl)phenyl]methyl]cyclohexyl]-[(3S)-3-(5-fluoro-3-pyridyl)isoxazolidin-2-yl]methanone,
(221) trans-3-fluoro-5-[(3S)-2-[4-[(2-methyl-[1,2,4]triazolo[1,5-a]pyridin-6-yl)methyl]cyclohexanecarbonyl]isoxazolidin-3-yl]benzonitrile,
(222) trans-[4-[(2-methyl-[1,2,4]triazolo[1,5-a]pyridin-6-yl)methyl]cyclohexyl]-[(3S)-3-pyrazin-2-ylisoxazolidin-2-yl]methanone,
(223) trans-[(3S)-3-(3,5-difluorophenyl)isoxazolidin-2-yl]-[4-[(2-methyl-[1,2,4]triazolo[1,5-a]pyridin-6-yl)methyl]cyclohexyl]methanone,
(224) trans-3-fluoro-5-[(3S)-2-[4-[(3-methyl-[1,2,4]triazolo[4,3-a]pyridin-6-yl)methyl]cyclohexanecarbonyl]isoxazolidin-3-yl]benzonitrile,
(225) trans-3-fluoro-5-[(3S)-2-[4-[(2-methylimidazo[1,2-a]pyridin-6-yl)methyl]cyclohexanecarbonyl]isoxazolidin-3-yl]benzonitrile,
(226) trans-[4-[(2-methylimidazo[1,2-a]pyridin-6-yl)methyl]cyclohexyl]-[(3S)-3-pyrazin-2-ylisoxazolidin-2-yl]methanone,
(227) trans-[(3S)-3-(3,5-difluorophenyl)isoxazolidin-2-yl]-[4-[(2-methylimidazo[1,2-a]pyridin-6-yl)methyl]cyclohexyl]methanone,
(228) trans-2-[[4-[(3S)-3-pyrazin-2-ylisoxazolidine-2-carbonyl]cyclohexyl]methyl]indazole-6-carboxamide,
(229) trans-1-[[4-[(3S)-3-pyrazin-2-ylisoxazolidine-2-carbonyl]cyclohexyl]methyl]indazole-6-carboxamide,
(230) trans-1-[[4-[(3S)-3-(2-pyridyl)isoxazolidine-2-carbonyl]cyclohexyl]methyl]indazole-6-carboxamide,
(231) trans-1-[[4-[(3S)-3-(4-cyano-2-pyridyl)isoxazolidine-2-carbonyl]cyclohexyl]methyl]indazole-6-carboxamide,
(232) trans-2-[[4-[(3S)-3-(4-cyano-2-pyridyl)isoxazolidine-2-carbonyl]cyclohexyl]methyl]indazole-6-carboxamide,
(233) trans-2-[[4-[(3S)-3-(2-methylthiazol-4-yl)isoxazolidine-2-carbonyl]cyclohexyl]methyl]indazole-6-carboxamide,
(234) trans-1-[[4-[(3S)-3-(2-methylthiazol-4-yl)isoxazolidine-2-carbonyl]cyclohexyl]methyl]indazole-6-carboxamide,
(235) trans-5-[(3S)-2-[4-[(1-methylpyrazol-4-yl)methyl]cyclohexanecarbonyl]isoxazolidin-3-yl]pyridine-3-carbonitrile,
(236) trans-[(3S)-3-(3,5-difluorophenyl)isoxazolidin-2-yl]-[4-[(1-methylpyrazol-4-yl)methyl]cyclohexyl]methanone,
(237) trans-3-fluoro-5-[(3S)-2-[4-[(2-methylpyrazol-3-yl)methyl]cyclohexanecarbonyl]isoxazolidin-3-yl]benzonitrile,
(238) trans-[4-[(2-methylpyrazol-3-yl)methyl]cyclohexyl]-[(3S)-3-pyrazin-2-ylisoxazolidin-2-yl]methanone,
(239) trans-3-fluoro-5-[(3S)-2-[4-[(2-methylthiazol-4-yl)methyl]cyclohexanecarbonyl]isoxazolidin-3-yl]benzonitrile,
(240) trans-[4-[(2-methylthiazol-4-yl)methyl]cyclohexyl]-[(3S)-3-pyrazin-2-ylisoxazolidin-2-yl]methanone,
(241) trans-1-[[4-[(3S)-3-(6-methoxypyrazin-2-yl)isoxazolidine-2-carbonyl]cyclohexyl]methyl]indole-6-carbonitrile,
(242) trans-2-fluoro-5-[[4-[(3S)-3-(4-fluorophenyl)isoxazolidine-2-carbonyl]cyclohexyl]methyl]benzonitrile,
(243) trans-2-fluoro-5-[[4-[(3S)-3-pyrazin-2-ylisoxazolidine-2-carbonyl]cyclohexyl]methyl]benzonitrile,
(244) trans-5-[[4-[(3S)-3-(3-cyano-5-fluoro-phenyl)isoxazolidine-2-carbonyl]cyclohexyl]methyl]-2-fluoro-benzonitrile,
(245) trans-3-fluoro-5-[(3S)-2-[4-(pyrazin-2-ylmethyl)cyclohexanecarbonyl]isoxazolidin-3-yl]benzonitrile,
(246) trans-[(3S)-3-pyrazin-2-ylisoxazolidin-2-yl]-[4-(pyrazin-2-ylmethyl)cyclohexyl]methanone,
(247) trans-3-fluoro-5-[(3S)-2-[4-(pyrimidin-5-ylmethyl)cyclohexanecarbonyl]isoxazolidin-3-yl]benzonitrile,
(248) trans-[(3S)-3-pyrazin-2-ylisoxazolidin-2-yl]-[4-(pyrimidin-5-ylmethyl)cyclohexyl]methanone,
(249) trans-3-fluoro-5-[(3S)-2-[4-[(8-methyl-[1,2,4]triazolo[1,5-a]pyridin-6-yl)methyl]cyclohexanecarbonyl]-1,2-oxazolidin-3-yl]benzonitrile,
(250) trans-[4-[(8-methyl-[1,2,4]triazolo[1,5-a]pyridin-6-yl)methyl]cyclohexyl]-[(3S)-3-pyrazin-2-yl-1,2-oxazolidin-2-yl]methanone,
(251) trans-1-[[4-[(3S)-3-pyrazin-2-ylisoxazolidine-2-carbonyl]cyclohexyl]methyl]indole-5-carboxamide,
(252) trans-1-[[4-[(3S)-3-(5-cyano-3-pyridyl)isoxazolidine-2-carbonyl]cyclohexyl]methyl]indole-5-carboxamide,
(253) trans-1-[[4-[(3S)-3-(6-methoxypyrazin-2-yl)isoxazolidine-2-carbonyl]cyclohexyl]methyl]indole-5-carboxamide,
(254) trans-1-[[4-[(3S)-3-(2-methylthiazol-4-yl)isoxazolidine-2-carbonyl]cyclohexyl]methyl]indole-5-carboxamide,
(255) trans-1-[[4-[(3S)-3-(2-pyridyl)isoxazolidine-2-carbonyl]cyclohexyl]methyl]indole-5-carboxamide,
(256) trans-3-fluoro-5-[(3S)-2-[4-[[3-fluoro-5-(hydroxymethyl)phenyl]methyl]cyclohexanecarbonyl]isoxazolidin-3-yl]benzonitrile,
(257) trans-5-[(3S)-2-[4-[[3-fluoro-5-(hydroxymethyl)phenyl]methyl]cyclohexanecarbonyl]isoxazolidin-3-yl]pyridine-3-carbonitrile,
(258) trans-3-fluoro-5-[(3S)-2-[4-[(1-methyltriazol-4-yl)methyl]cyclohexanecarbonyl]isoxazolidin-3-yl]benzonitrile,
(259) trans-[4-[[3-fluoro-5-(hydroxymethyl)phenyl]methyl]cyclohexyl]-[(3S)-3-pyrazin-2-ylisoxazolidin-2-yl]methanone,
(260) trans-[(3S)-3-(4-fluorophenyl)isoxazolidin-2-yl]-[4-[(2-methyl-[1,2,4]triazolo[1,5-a]pyridin-6-yl)methyl]cyclohexyl]methanone,
(261) trans-[(3S)-3-(3,4-difluorophenyl)isoxazolidin-2-yl]-[4-[(2-methyl-[1,2,4]triazolo[1,5-a]pyridin-6-yl)methyl]cyclohexyl]methanone,
(262) trans-5-[(3S)-2-[4-[(2-methyl-[1,2,4]triazolo[1,5-a]pyridin-6-yl)methyl]cyclohexanecarbonyl]isoxazolidin-3-yl]pyridine-3-carbonitrile,
(263) trans-3-fluoro-5-[(3S)-2-[4-[(2-methylpyrimidin-5-yl)methyl]cyclohexanecarbonyl]isoxazolidin-3-yl]benzonitrile,
(264) trans-[(3S)-3-(3,5-difluorophenyl)isoxazolidin-2-yl]-[4-[(2-methylpyrimidin-5-yl)methyl]cyclohexyl]methanone,
(265) trans-[(3S)-3-(3,5-difluorophenyl)isoxazolidin-2-yl]-[4-[[3-fluoro-4-(hydroxymethyl)phenyl]methyl]cyclohexyl]methanone,
(266) trans-3-fluoro-5-[(3S)-2-[4-[[3-fluoro-4-(hydroxymethyl)phenyl]methyl]cyclohexanecarbonyl]isoxazolidin-3-yl]benzonitrile,
(267) trans-5-[[4-[(3S)-3-(3,5-difluorophenyl)isoxazolidine-2-carbonyl]cyclohexyl]methyl]-2-methyl-pyridine-3-carbonitrile,
(268) trans-5-[[4-[(3S)-3-(3-cyano-5-fluoro-phenyl)isoxazolidine-2-carbonyl]cyclohexyl]methyl]-2-methyl-pyridine-3-carbonitrile,
(269) 5-[[4-[(3S)-3-(3,5-difluorophenyl)isoxazolidine-2-carbonyl]norbornan-1-yl]methyl]-2-methyl-benzamide,
(270) 5-[[4-[(3S)-3-(3-cyano-5-fluoro-phenyl)isoxazolidine-2-carbonyl]norbornan-1-yl]methyl]-2-methyl-benzamide,
(271) 2-methyl-5-[[4-[(3S)-3-(6-methyl-3-pyridyl)isoxazolidine-2-carbonyl]norbornan-1-yl]methyl]benzamide,
(272) 2-methyl-5-[[4-[(3S)-3-(6-methyl-3-pyridyl)isoxazolidine-2-carbonyl]-1-bicyclo[2.2.2]octanyl]methyl]benzamide,
(273) 5-[[4-[(3S)-3-(3,5-difluorophenyl)-1,2-oxazolidine-2-carbonyl]-1-bicyclo[2.2.2]octanyl]methyl]-2-methylbenzamide,
(274) 5-[[4-[(3S)-3-(3-cyano-5-fluoro-phenyl)isoxazolidine-2-carbonyl]-1-bicyclo[2.2.2]octanyl]methyl]-2-methyl-benzamide,
(275) trans-5-[[4-[(3S)-3-(3,5-difluorophenyl)isoxazolidine-2-carbonyl]cyclohexyl]methyl]-4-methyl-pyridine-3-carbonitrile,
(276) trans-5-[[4-[(3S)-3-(3-cyano-5-fluoro-phenyl)isoxazolidine-2-carbonyl]cyclohexyl]methyl]-4-methyl-pyridine-3-carbonitrile,
(277) trans-5-[[4-[(3S)-3-(3-cyano-5-fluoro-phenyl)isoxazolidine-2-carbonyl]cyclohexyl]methyl]-6-methyl-pyridine-3-carbonitrile,
(278) trans-5-[[4-[(3S)-3-(3,5-difluorophenyl)isoxazolidine-2-carbonyl]cyclohexyl]methyl]-6-methyl-pyridine-3-carbonitrile formic acid salt,
(279) trans-3-fluoro-5-[(3S)-2-[4-[[3-fluoro-4-(2-hydroxyethyl)phenyl]methyl]cyclohexanecarbonyl]isoxazolidin-3-yl]benzonitrile,
(280) trans-[(3S)-3-(3,5-difluorophenyl)isoxazolidin-2-yl]-[4-[[3-fluoro-4-(2-hydroxyethyl)phenyl]methyl]cyclohexyl]methanone,
(281) trans-[(3S)-3-(3,5-difluorophenyl)isoxazolidin-2-yl]-[4-[(8-fluoro-2-methyl-imidazo[1,2-a]pyridin-6-yl)methyl]cyclohexyl]methanone,
(282) trans-3-fluoro-5-[(3S)-2-[4-[[3-fluoro-5-(2-hydroxyethyl)phenyl]methyl]cyclohexanecarbonyl]isoxazolidin-3-yl]benzonitrile,
(283) trans-[(3S)-3-(3,5-difluorophenyl)isoxazolidin-2-yl]-[4-[[3-fluoro-5-(2-hydroxyethyl)phenyl]methyl]cyclohexyl]methanone,
(284) trans-3-fluoro-5-[(3S)-2-[4-[(8-fluoro-2-methyl-[1,2,4]triazolo[1,5-a]pyridin-6-yl)methyl]cyclohexanecarbonyl]isoxazolidin-3-yl]benzonitrile,
(285) trans-[(3S)-3-(3,5-difluorophenyl)isoxazolidin-2-yl]-[4-[(8-fluoro-2-methyl-[1,2,4]triazolo[1,5-a]pyridin-6-yl)methyl]cyclohexyl]methanone,
(286) trans-5-[[4-[(3S)-3-(3-cyano-5-fluoro-4-methyl-phenyl)isoxazolidine-2-carbonyl]cyclohexyl]methyl]-2-methyl-pyridine-3-carbonitrile,
(287) trans-5-[[4-[(3S)-3-(3-cyano-5-fluoro-4-methyl-phenyl)isoxazolidine-2-carbonyl]cyclohexyl]methyl]-4-methyl-pyridine-3-carbonitrile,
(288) trans-3-[(3S)-2-[4-[(2,8-dimethyl-[1,2,4]triazolo[1,5-a]pyridin-6-yl)methyl]cyclohexanecarbonyl]isoxazolidin-3-yl]-5-fluoro-benzonitrile,
(289) trans-4-fluoro-3-[(3S)-2-[4-[(2-methyl-[1,2,4]triazolo[1,5-a]pyridin-6-yl)methyl]cyclohexanecarbonyl]isoxazolidin-3-yl]benzonitrile,
(290) trans-3-[(3S)-2-[4-[(2,8-dimethylimidazo[1,2-b]pyridazin-6-yl)methyl]cyclohexanecarbonyl]isoxazolidin-3-yl]-5-fluoro-benzonitrile,
(291) trans-[(3S)-3-(3,5-difluorophenyl)isoxazolidin-2-yl]-[4-[(2,8-dimethylimidazo[1,2-b]pyridazin-6-yl)methyl]cyclohexyl]methanone,
(292) trans-3-[(3S)-2-[4-[(2,7-dimethylimidazo[1,2-b]pyridazin-6-yl)methyl]cyclohexanecarbonyl]isoxazolidin-3-yl]-5-fluoro-benzonitrile,
(293) trans-[(3S)-3-(3,5-difluorophenyl)isoxazolidin-2-yl]-[4-[(2,7-dimethylimidazo[1,2-b]pyridazin-6-yl)methyl]cyclohexyl]methanone,
(294) trans-3-fluoro-5-[(3S)-2-[4-[(1-methylindazol-6-yl)methyl]cyclohexanecarbonyl]isoxazolidin-3-yl]benzonitrile,
(295) trans-[(3S)-3-(3,5-difluorophenyl)isoxazolidin-2-yl]-[4-[(1-methylindazol-6-yl)methyl]cyclohexyl]methanone,
(296) trans-3-fluoro-5-[(3S)-2-[4-[(1-methylpyrazolo[4,3-b]pyridin-6-yl)methyl]cyclohexanecarbonyl]isoxazolidin-3-yl]benzonitrile,
(297) trans-[(3S)-3-(3,5-difluorophenyl)isoxazolidin-2-yl]-[4-[(1-methylpyrazolo[4,3-b]pyridin-6-yl)methyl]cyclohexyl]methanone,
(298) 6-fluoro-1-[[trans-4-[(3S)-3-(6-methyl-3-pyridyl)isoxazolidine-2-carbonyl]cyclohexyl]methyl]indazole-5-carbonitrile,
(299) trans-2-fluoro-5-[[4-[(3S)-3-(6-methylpyrazin-2-yl)isoxazolidine-2-carbonyl]cyclohexyl]methyl]benzamide,
(300) trans-3-[(3S)-2-[4-[(3,8-dimethyl-[1,2,4]triazolo[4,3-a]pyridin-6-yl)methyl]cyclohexanecarbonyl]-1,2-oxazolidin-3-yl]-5-fluorobenzonitrile,
(301) trans-[4-[(3,8-dimethyl-[1,2,4]triazolo[4,3-a]pyridin-6-yl)methyl]cyclohexyl]-[(3S)-3-pyrazin-2-yl-1,2-oxazolidin-2-yl]methanone,
(302) trans-3-fluoro-5-[(3S)-2-[4-[(6-methylpyridazin-4-yl)methyl]cyclohexanecarbonyl]isoxazolidin-3-yl]benzonitrile,
(303) trans-3-fluoro-5-[(3S)-2-[4-[(6-methylpyridazin-3-yl)methyl]cyclohexanecarbonyl]isoxazolidin-3-yl]benzonitrile,
(304) trans-[(3S)-3-(3,5-difluorophenyl)isoxazolidin-2-yl]-[4-[(6-methylpyridazin-3-yl)methyl]cyclohexyl]methanone,
(305) trans-[4-[[5-(2,5-dimethylpyrazol-3-yl)-3-pyridyl]methyl]cyclohexyl]-[(3S)-3-pyrazin-2-ylisoxazolidin-2-yl]methanone,
(306) trans-[(3S)-3-(4-fluorophenyl)isoxazolidin-2-yl]-[4-[(2-methylpyrazol-3-yl)methyl]cyclohexyl]methanone,
(307) trans-[(3S)-3-(3,5-difluorophenyl)isoxazolidin-2-yl]-[4-[(2-methylpyrazol-3-yl)methyl]cyclohexyl]methanone,
(308) trans-[4-[[5-(1,3-dimethylpyrazol-4-yl)-3-pyridyl]methyl]cyclohexyl]-[(3S)-3-pyrazin-2-ylisoxazolidin-2-yl]methanone,
(309) trans-[(3S)-3-(3,4-difluorophenyl)isoxazolidin-2-yl]-[4-[(2-methylpyrimidin-5-yl)methyl]cyclohexyl]methanone,
(310) trans-[(3S)-3-(3,4-difluorophenyl)isoxazolidin-2-yl]-[4-[(2-methyl-[1,2,4]triazolo[1,5-b]pyridazin-6-yl)methyl]cyclohexyl]methanone,
(311) trans-[(3S)-3-(3,4-difluorophenyl)isoxazolidin-2-yl]-[4-[(2-methylpyrazol-3-yl)methyl]cyclohexyl]methanone,
(312) trans-3-[(3S)-2-[4-[(2,6-dimethylpyrimidin-4-yl)methyl]cyclohexanecarbonyl]isoxazolidin-3-yl]-5-fluoro-benzonitrile,
(313) trans-[(3S)-3-(3,5-difluorophenyl)isoxazolidin-2-yl]-[4-[(6-methylpyrimidin-4-yl)methyl]cyclohexyl]methanone,
(314) trans-[4-[(8-methoxy-2-methyl-[1,2,4]triazolo[1,5-a]pyridin-6-yl)methyl]cyclohexyl]-[(3S)-3-pyrazin-2-yl-1,2-oxazolidin-2-yl]methanone,
(315) trans-[(3S)-3-(3,5-difluorophenyl)-1,2-oxazolidin-2-yl]-[4-[(2-methyl-[1,2,4]triazolo[1,5-b]pyridazin-6-yl)methyl]cyclohexyl]methanone,
(316) trans-[4-[(5-fluoro-2-methyl-3-pyridyl)methyl]cyclohexyl]-[(3S)-3-(5-methylpyrazin-2-yl)isoxazolidin-2-yl]methanone,
(317) trans-[4-[(3,7-dimethyl-[1,2,4]triazolo[4,3-a]pyridin-6-yl)methyl]cyclohexyl]-[(3S)-3-(3-fluorophenyl)isoxazolidin-2-yl]methanone,
(318) trans-[(3S)-3-(3,4-difluorophenyl)isoxazolidin-2-yl]-[4-[(3,7-dimethyl-[1,2,4]triazolo[4,3-a]pyridin-6-yl)methyl]cyclohexyl]methanone,
(319) trans-[4-[(3,8-dimethyl-[1,2,4]triazolo[4,3-a]pyridin-6-yl)methyl]cyclohexyl]-[(3S)-3-(4-fluorophenyl)isoxazolidin-2-yl]methanone,
(320) trans-[4-[(8-fluoro-3-methyl-[1,2,4]triazolo[4,3-a]pyridin-6-yl)methyl]cyclohexyl]-[(3S)-3-(4-fluorophenyl)isoxazolidin-2-yl]methanone,
(321) trans-[(3S)-3-(4-fluorophenyl)isoxazolidin-2-yl]-[4-[(2-methyl-[1,2,4]triazolo[1,5-a]pyridin-7-yl)methyl]cyclohexyl]methanone,
(322) trans-[2-cyano-4-[(3S)-2-[4-[(2-methylimidazo[1,2-b]pyridazin-7-yl)methyl]cyclohexanecarbonyl]isoxazolidin-3-yl]phenyl],
(323) trans-[2-fluoro-4-[(3S)-2-[4-[(2-methylimidazo[1,2-b]pyridazin-7-yl)methyl]cyclohexanecarbonyl]isoxazolidin-3-yl]phenyl],
(324) trans-[4-[(3S)-2-[4-[(2-methylimidazo[1,2-b]pyridazin-7-yl)methyl]cyclohexanecarbonyl]isoxazolidin-3-yl]phenyl],
(325) trans-[4-[(2,8-dimethyl-[1,2,4]triazolo[1,5-a]pyridin-6-yl)methyl]cyclohexyl]-[(3S)-3-(6-methyl-3-pyridyl)isoxazolidin-2-yl]methanone,
(326) trans-[4-[(2,8-dimethyl-[1,2,4]triazolo[1,5-a]pyridin-6-yl)methyl]cyclohexyl]-[(3S)-3-pyrazin-2-ylisoxazolidin-2-yl]methanone,
(327) trans-[(3S)-3-(5-fluoro-6-methyl-3-pyridyl)isoxazolidin-2-yl]-[4-[(8-fluoro-2-methyl-[1,2,4]triazolo[1,5-a]pyridin-6-yl)methyl]cyclohexyl]methanone,
(328) trans-[4-[(2,8-dimethyl-[1,2,4]triazolo[1,5-a]pyridin-6-yl)methyl]cyclohexyl]-[(3S)-3-(5-methylpyrazin-2-yl)isoxazolidin-2-yl]methanone,
(329) trans-[4-[(1-methylpyrazolo[4,3-b]pyridin-6-yl)methyl]cyclohexyl]-[(3S)-3-(6-methylpyridin-3-yl)-1,2-oxazolidin-2-yl]methanone,
(330) trans-[4-[(2,8-dimethyl-[1,2,4]triazolo[1,5-a]pyridin-6-yl)methyl]cyclohexyl]-[(3S)-3-(5-fluoro-6-methylpyridin-3-yl)-1,2-oxazolidin-2-yl]methanone,
(331) trans-[4-[(2,8-dimethyl-[1,2,4]triazolo[1,5-a]pyridin-6-yl)methyl]cyclohexyl]-[(3S)-3-(5-fluoropyridin-3-yl)-1,2-oxazolidin-2-yl]methanone,
(332) trans-[4-[(2,5-dimethylpyridin-4-yl)methyl]cyclohexyl]-[(3S)-3-(5-fluoro-6-methylpyridin-3-yl)-1,2-oxazolidin-2-yl]methanone,
(333) trans-[4-[(8-fluoro-2-methyl-[1,2,4]triazolo[1,5-a]pyridin-6-yl)methyl]cyclohexyl]-[(3S)-3-(5-methylpyridin-3-yl)-1,2-oxazolidin-2-yl]methanone,
(334) trans-[(3S)-3-(5-methylpyridin-3-yl)-1,2-oxazolidin-2-yl]-[4-[(8-methyl-[1,2,4]triazolo[1,5-a]pyridin-6-yl)methyl]cyclohexyl]methanone,
(335) trans-[(3S)-3-(5-fluoro-6-methylpyridin-3-yl)-1,2-oxazolidin-2-yl]-[4-[(8-methyl-[1,2,4]triazolo[1,5-a]pyridin-6-yl)methyl]cyclohexyl]methanone,
(336) trans-[(3S)-3-(5-fluoropyridin-3-yl)-1,2-oxazolidin-2-yl]-[4-[(8-methyl-[1,2,4]triazolo[1,5-a]pyridin-6-yl)methyl]cyclohexyl]methanone,
(337) trans-[(3S)-3-(5-fluoro-6-methylpyridin-3-yl)-1,2-oxazolidin-2-yl]-[4-([1,2,4]triazolo[1,5-a]pyridin-6-ylmethyl)cyclohexyl]methanone,
(338) trans-[(3S)-3-(5-fluoropyridin-3-yl)-1,2-oxazolidin-2-yl]-[4-([1,2,4]triazolo[1,5-a]pyridin-6-ylmethyl)cyclohexyl]methanone,
(339) trans-[4-[(2,5-dimethylpyridin-4-yl)methyl]cyclohexyl]-[(3S)-3-(5-methylpyrazin-2-yl)-1,2-oxazolidin-2-yl]methanone,
(340) trans-[4-[(8-fluoro-2-methyl-[1,2,4]triazolo[1,5-a]pyridin-6-yl)methyl]cyclohexyl]-[(3S)-3-pyrazin-2-yl-1,2-oxazolidin-2-yl]methanone,
(341) trans-[4-[(8-fluoro-2-methyl-[1,2,4]triazolo[1,5-a]pyridin-6-yl)methyl]cyclohexyl]-[(3S)-3-(6-methylpyridin-3-yl)-1,2-oxazolidin-2-yl]methanone,
(342) trans-[(3S)-3-(5-fluoro-6-methylpyridin-3-yl)-1,2-oxazolidin-2-yl]-[4-[(8-methoxy-2-methyl-[1,2,4]triazolo[1,5-a]pyridin-6-yl)methyl]cyclohexyl]methanone,
(343) trans-[(3S)-3-(5-fluoro-6-methylpyridin-3-yl)-1,2-oxazolidin-2-yl]-[4-[(2-methylpyrimidin-5-yl)methyl]cyclohexyl]methanone,
(344) trans-[(3S)-3-(6-methylpyridin-3-yl)-1,2-oxazolidin-2-yl]-[4-[(2-methylpyrimidin-5-yl)methyl]cyclohexyl]methanone,
(345) trans-[(3S)-3-(5-fluoropyridin-3-yl)-1,2-oxazolidin-2-yl]-[4-[(8-methoxy-2-methyl-[1,2,4]triazolo[1,5-a]pyridin-6-yl)methyl]cyclohexyl]methanone,
(346) trans-[4-[(2-methylpyrimidin-5-yl)methyl]cyclohexyl]-[(3S)-3-pyrazin-2-yl-1,2-oxazolidin-2-yl]methanone,
(347) trans-[(3S)-3-(6-methylpyridin-3-yl)-1,2-oxazolidin-2-yl]-[4-[(8-methyl-[1,2,4]triazolo[1,5-a]pyridin-6-yl)methyl]cyclohexyl]methanone,
(348) trans-[4-[[4-(3-hydroxyoxetan-3-yl)phenyl]methyl]cyclohexyl]-[(3S)-3-(6-methylpyridin-3-yl)-1,2-oxazolidin-2-yl]methanone,
(349) trans-[4-[(2-methylimidazo[1,2-b]pyridazin-7-yl)methyl]cyclohexyl]-[(3S)-3-pyrazin-2-yl-1,2-oxazolidin-2-yl]methanone,
(350) trans-[4-(8-Fluoro-2-methyl-imidazo[1,2-a]pyridin-6-ylmethyl)-cyclohexyl]-((S)-3-pyrazin-2-yl-isoxazolidin-2-yl)-methanone,
(351) trans-[4-[(8-fluoro-2-methylimidazo[1,2-a]pyridin-6-yl)methyl]cyclohexyl]-[(3S)-3-(6-methylpyridin-3-yl)-1,2-oxazolidin-2-yl]methanone,
(352) trans-[4-[[4-(3-hydroxyoxetan-3-yl)phenyl]methyl]cyclohexyl]-[(3S)-3-pyrazin-2-yl-1,2-oxazolidin-2-yl]methanone,
(353) trans-[4-[(2-methylimidazo[1,2-b]pyridazin-7-yl)methyl]cyclohexyl]-[(3S)-3-(6-methylpyridin-3-yl)-1,2-oxazolidin-2-yl]methanone,
(354) trans-[4-(2,5-Dimethyl-pyridin-4-ylmethyl)-cyclohexyl]-[(S)-3-(6-methyl-pyridin-3-yl)-isoxazolidin-2-yl]-methanone,
(355) trans-[4-[(2-methyl-[1,2,4]triazolo[1,5-a]pyridin-7-yl)methyl]cyclohexyl]-[(3S)-3-(5-fluoro-6-methyl-3-pyridyl)isoxazolidin-2-yl]-methanone
(356) trans-[4-(2,5-Dimethyl-pyridin-4-ylmethyl)-cyclohexyl]-((S)-3-pyrazin-2-yl-isoxazolidin-2-yl)-methanone,
(357) trans-[4-[(6-methylpyridazin-3-yl)methyl]cyclohexyl]-[(3S)-3-pyrazin-2-ylisoxazolidin-2-yl]methanone,
(358) trans-[4-[(3,5-dimethyl-[1,2,4]triazolo[4,3-a]pyridin-6-yl)methyl]cyclohexyl]-[(3S)-3-pyrazin-2-yl-1,2-oxazolidin-2-yl]methanone,
(359) trans-[4-[(8-fluoro-3-methyl-[1,2,4]triazolo[4,3-a]pyridin-6-yl)methyl]cyclohexyl]-[(3S)-3-pyrazin-2-yl-1,2-oxazolidin-2-yl]methanone,
(360) trans-(S)-3-(5-fluoro-6-methylpyridin-3-yl)-1,2-oxazolidin-2-yl]-[4-[(2-methyl-1,3-thiazol-4-yl)methyl]cyclohexyl]methanone,
(361) trans-[4-(2,8-Dimethyl-[1,2,4]triazolo[1,5-a]pyridin-6-ylmethyl)-cyclohexyl]-[(S)-3-(2-methyl-thiazol-4-yl)-isoxazolidin-2-yl]-methanone,
(362) trans-[(S)-3-(2-Methyl-thiazol-4-yl)-isoxazolidin-2-yl]-[4-(8-methyl-[1,2,4]triazolo[1,5-a]pyridin-6-ylmethyl)-cyclohexyl]-methanone,
(363) trans-[4-(8-Fluoro-3-methyl-[1,2,4]triazolo[4,3-a]pyridin-6-ylmethyl)-cyclohexyl]-[(S)-3-(2-methyl-thiazol-4-yl)-isoxazolidin-2-yl]-methanone,
(364) trans-[4-(8-Fluoro-3-methyl-[1,2,4]triazolo[4,3-a]pyridin-6-ylmethyl)-cyclohexyl]-[(S)-3-(6-methyl-pyridin-3-yl)-isoxazolidin-2-yl]-methanone,
(365) trans-[4-(3,8-Dimethyl-[1,2,4]triazolo[4,3-a]pyridin-6-ylmethyl)-cyclohexyl]-[(S)-3-(6-methyl-pyridin-3-yl)-isoxazolidin-2-yl]-methanone,
(366) trans-[4-(2,5-Dimethyl-pyridin-4-ylmethyl)-cyclohexyl]-[(S)-3-(2-methyl-thiazol-4-yl)-isoxazolidin-2-yl]-methanone,
(367) trans-[4-(8-Methoxy-2-methyl-[1,2,4]triazolo[1,5-a]pyridin-6-ylmethyl)-cyclohexyl]-[(S)-3-(2-methyl-thiazol-4-yl)-isoxazolidin-2-yl]-methanone,
(368) trans-[4-(8-Methoxy-2-methyl-[1,2,4]triazolo[1,5-a]pyridin-6-ylmethyl)-cyclohexyl]-[(S)-3-(6-methyl-pyridin-3-yl)-isoxazolidin-2-yl]-methanone,
(369) trans-{4-[4-(3-Hydroxy-oxetan-3-yl)-benzyl]-cyclohexyl}-[(S)-3-(2-methyl-thiazol-4-yl)-isoxazolidin-2-yl]-methanone,
(370) trans-[4-(8-Fluoro-2-methyl-imidazo[1,2-a]pyridin-6-ylmethyl)-cyclohexyl]-[(S)-3-(2-methyl-thiazol-4-yl)-isoxazolidin-2-yl]-methanone,
(371) trans-[4-(2-Methyl-imidazo[1,2-b]pyridazin-7-ylmethyl)-cyclohexyl]-[(S)-3-(2-methyl-thiazol-4-yl)-isoxazolidin-2-yl]-methanone,
(372) trans-[(S)-3-(2-Methyl-thiazol-4-yl)-isoxazolidin-2-yl]-[4-(2-methyl-[1,2,4]triazolo[1,5-a]pyridin-7-ylmethyl)-cyclohexyl]-methanone,
(373) trans-[(S)-3-(6-Methyl-pyridin-3-yl)-isoxazolidin-2-yl]-[4-(2-methyl-[1,2,4]triazolo[1,5-a]pyridin-7-ylmethyl)-cyclohexyl]-methanone,
(374) trans-[4-(2-Methyl-[1,2,4]triazolo[1,5-a]pyridin-7-ylmethyl)-cyclohexyl]-((S)-3-pyrazin-2-yl-isoxazolidin-2-yl)-methanone,
(375) trans-[4-(3,5-Dimethyl-[1,2,4]triazolo[4,3-a]pyridin-6-ylmethyl)-cyclohexyl]-[(S)-3-(2-methyl-thiazol-4-yl)-isoxazolidin-2-yl]-methanone,
(376) trans-[4-(3,5-Dimethyl-[1,2,4]triazolo[4,3-a]pyridin-6-ylmethyl)-cyclohexyl]-[(S)-3-(6-methyl-pyridin-3-yl)-isoxazolidin-2-yl]-methanone,
(377) trans-[4-[(2,5-dimethylpyridin-4-yl)methyl]cyclohexyl]-[(3S)-3-(2-methyl-1,3-oxazol-4-yl)-1,2-oxazolidin-2-yl]methanone,
(378) trans-[4-[(2,8-dimethyl-[1,2,4]triazolo[1,5-a]pyridin-6-yl)methyl]cyclohexyl]-[(3S)-3-(5-methylpyridin-3-yl)-1,2-oxazolidin-2-yl]methanone,
(379) trans-[4-[(8-fluoro-2-methyl-[1,2,4]triazolo[1,5-a]pyridin-6-yl)methyl]cyclohexyl]-[(3S)-3-(5-methylpyrazin-2-yl)-1,2-oxazolidin-2-yl]methanone,
(380) trans-[4-[(8-fluoro-2-methyl-[1,2,4]triazolo[1,5-a]pyridin-6-yl)methyl]cyclohexyl]-[(3S)-3-(5-fluoropyridin-3-yl)-1,2-oxazolidin-2-yl]methanone,
(381) trans-[4-[(8-chloro-2-methyl-[1,2,4]triazolo[1,5-a]pyridin-6-yl)methyl]cyclohexyl]-[(3S)-3-(5-fluoro-6-methylpyridin-3-yl)-1,2-oxazolidin-2-yl]methanone,
(382) trans-[4-[(8-chloro-2-methyl-[1,2,4]triazolo[1,5-a]pyridin-6-yl)methyl]cyclohexyl]-[(3S)-3-(5-fluoropyridin-3-yl)-1,2-oxazolidin-2-yl]methanone,
(383) trans-[4-[(8-methoxy-2-methyl-[1,2,4]triazolo[1,5-a]pyridin-6-yl)methyl]cyclohexyl]-[(3S)-3-(5-methylpyridin-3-yl)-1,2-oxazolidin-2-yl]methanone,
(384) trans-[4-[(5-fluoro-2-methylpyridin-4-yl)methyl]cyclohexyl]-[(3S)-3-(5-methylpyrazin-2-yl)-1,2-oxazolidin-2-yl]methanone,
(385) trans-[4-[(5-chloro-2-methylpyridin-4-yl)methyl]cyclohexyl]-[(3S)-3-(5-methylpyrazin-2-yl)-1,2-oxazolidin-2-yl]methanone,
(386) trans-[4-[(5-fluoro-2-methylpyridin-4-yl)methyl]cyclohexyl]-[(3S)-3-(6-methylpyridin-3-yl)-1,2-oxazolidin-2-yl]methanone,
(387) trans-[4-[(6-methylpyridazin-3-yl)methyl]cyclohexyl]-[(3S)-3-(6-methylpyridin-3-yl)-1,2-oxazolidin-2-yl]methanone,
(388) trans-[4-[(6-methylpyridazin-3-yl)methyl]cyclohexyl]-[(3S)-3-(5-fluoro-6-methylpyridin-3-yl)-1,2-oxazolidin-2-yl]methanone,
(389) trans-[4-[(5-chloro-2-methylpyridin-4-yl)methyl]cyclohexyl]-[(3S)-3-(6-methylpyridin-3-yl)-1,2-oxazolidin-2-yl]methanone,
(390) trans-[4-[(8-fluoro-[1,2,4]triazolo[1,5-a]pyridin-6-yl)methyl]cyclohexyl]-[(3S)-3-(5-methylpyridin-3-yl)-1,2-oxazolidin-2-yl]methanone,
(391) trans-[4-[(2-amino-[1,2,4]triazolo[1,5-a]pyridin-6-yl)methyl]cyclohexyl]-[(3S)-3-(6-methylpyridin-3-yl)-1,2-oxazolidin-2-yl]methanone,
(392) trans-[(3S)-3-(5-fluoro-6-methylpyridin-3-yl)-1,2-oxazolidin-2-yl]-[4-[(8-fluoro-[1,2,4]triazolo[1,5-a]pyridine-6-yl)methyl]cyclohexyl]methanone,
(393) trans-[4-[(2,4-dimethylpyrimidin-5-yl)methyl]cyclohexyl]-[(3S)-3-(5-fluoro-6-methylpyridin-3-yl)-1,2-oxazolidin-2-yl]methanone,
(394) trans-[4-[(5-fluoro-2-methylpyridin-4-yl)methyl]cyclohexyl]-[(3S)-3-pyrazin-2-yl-1,2-oxazolidin-2-yl]methanone,
(395) trans-[(3S)-3-(5-methylpyrazin-2-yl)-1,2-oxazolidin-2-yl]-[4-[(2-methylpyrazol-3-yl)methyl]cyclohexyl]methanone,
(396) trans-[4-[(5-chloro-2-methylpyridin-4-yl)methyl]cyclohexyl]-[(3S)-3-pyrazin-2-yl-1,2-oxazolidin-2-yl]methanone,
(397) trans-[4-[(2-methylpyrazol-3-yl)methyl]cyclohexyl]-[(3S)-3-(6-methylpyridin-3-yl)-1,2-oxazolidin-2-yl]methanone,
(398) trans-[(3S)-3-(5-fluoro-6-methylpyridin-3-yl)-1,2-oxazolidin-2-yl]-[4-[(6-methylpyridazin-4-yl)methyl]cyclohexyl]methanone,
(399) trans-[(3S)-3-(5-fluoro-6-methylpyridin-3-yl)-1,2-oxazolidin-2-yl]-[4-[[2-methyl-4-(trifluoromethyl)pyrimidin-5-yl]methyl]cyclohexyl]methanone,
(400) trans-[4-[(2,8-dimethyl-[1,2,4]triazolo[1,5-a]pyridin-6-yl)methyl]-1-methylcyclohexyl]-[(3S)-3-(6-methylpyridin-3-yl)-1,2-oxazolidin-2-yl]methanone,
(401) trans-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone,
(402) cis-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone,
(403) cis-[4-[(2,8-dimethyl-[1,2,4]triazolo[1,5-a]pyridin-6-yl)methyl]-1-methylcyclohexyl]-[(3S)-3-(5-fluoro-6-methylpyridin-3-yl)-1,2-oxazolidin-2-yl]methanone,
(404) trans-[(3S)-3-(5-fluoro-6-methylpyridin-3-yl)-1,2-oxazolidin-2-yl]-[4-[(2-methylpyrazol-3-yl)methyl]cyclohexyl]methanone,
(405) cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone,
(406) cis-[4-[(2,8-dimethyl-[1,2,4]triazolo[1,5-a]pyridin-6-yl)methyl]-4-methylcyclohexyl]-[(3S)-3-(5-fluoro-6-methylpyridin-3-yl)-1,2-oxazolidin-2-yl]methanone,
(407) cis-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone,
(408) trans-[4-[(2,8-dimethyl-[1,2,4]triazolo[1,5-a]pyridin-6-yl)methyl]-4-methylcyclohexyl]-[(3S)-3-(6-methylpyridin-3-yl)-1,2-oxazolidin-2-yl]methanone,
(409) trans-[(3S)-3-(5-methylpyrazin-2-yl)-1,2-oxazolidin-2-yl]-[4-[(8-methyl-[1,2,4]triazolo[1,5-a]pyridin-6-yl)methyl]cyclohexyl]methanone,
(410) trans-[4-[(1,3-dimethylpyrazolo[4,3-b]pyridin-6-yl)methyl]cyclohexyl]-[(3S)-3-(5-methylpyrazin-2-yl)-1,2-oxazolidin-2-yl]methanone, and
(411) trans-[4-[(2,8-dimethyl-[1,2,4]triazolo[1,5-a]pyridin-6-yl)methyl]cyclohexyl]-[(3S)-3-(6-methylpyrazin-2-yl)isoxazolidin-2-yl]methanone;
or a pharmaceutically acceptable salt, solvate, or stereoisomer thereof.

15. A pharmaceutical composition comprising a compound of formula (I) as defined in claim 1 or a pharmaceutically acceptable salt, solvate, or stereoisomer thereof and at least one pharmaceutically acceptable excipient.

16. A pharmaceutical composition comprising a compound according to claim 14, or a pharmaceutically acceptable salt, solvate, or stereoisomer thereof and at least one pharmaceutically acceptable excipient.

17. (canceled)

18. A method of treating and/or preventing a disease, disorder, or condition that is at least partly mediated by receptor-interacting protein kinase 1 (RIPK1) in a subject in need thereof, the method comprising administering to the subject a therapeutically effective amount of a compound of formula (I) according to claim 1, or a pharmaceutically acceptable salt, solvate, or stereoisomer thereof.

19. A method of treating and/or preventing a disease selected from Parkinson's disease, Alzheimer's disease, amyotrophic lateral sclerosis (ALS), or multiple sclerosis (MS) in a subject in need thereof, the method comprising administering to the subject a therapeutically effective amount of a compound of formula (I) according to claim 1, or a pharmaceutically acceptable salt, solvate, or stereoisomer thereof.

20. The compound of formula (Ic) as defined in claim 9, wherein:
R1 is a phenyl, a pyrazinyl, a pyridinyl, a thiazolyl, an oxazolyl or a pyrimidinyl, which is optionally substituted by one or two R3;
R2 is an imidazopyridazinyl, indazolyl, pyrazolyl, pyridinyl, pyrimidinyl, triazolopyridazinyl, triazolopyridinyl, pyrazolopyridinyl, imidazopyridinyl, phenyl, pyridazinyl, thiazolyl, optionally substituted by one or two R6;
each R3 is independently chosen from fluorine, cyano, methoxy or methyl;
m and s are 0 or m is 0 and s is 1 and R4 is a methyl group;
each R6 is independently chosen from fluorine, cyano, methyl, methoxy or hydroxyoxetanyl,
R7 is independently a halogen, oxo, —OH, a methyl or a methoxy,
or a pharmaceutically acceptable salt, solvate, or stereoisomer thereof.

21. The compound of formula (I) as defined in claim 1, having following formula (Id) or (Ie): 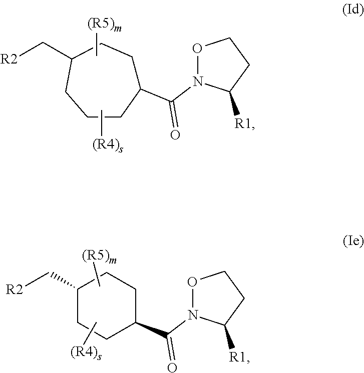
or a pharmaceutically acceptable salt, solvate, or stereoisomer thereof.
